(19) United States
(12) Patent Application Publication
(10) Pub. No.: US 2026/0308159 A1
(43) Pub. Date: Oct. 8, 2026

(54) BICYCLIC HETEROARYLAMIDES HAVING GPR68 MODULATING ACTIVITY

(71) Applicant: RECURSION PHARMACEUTICALS, INC., Salt Lake City, UT (US)

(72) Inventors: Simon Richards, Oxford (GB); Michael Prime, Oxford (GB); Konstantinos Papachristos, Oxford (GB); Manisha Naik, Oxford (GB); Reid Hans Johnson Olsen, Oxford (GB)

(21) Appl. No.: 19/484,697

(22) PCT Filed: May 31, 2024
(86) PCT No.: PCT/GB2024/051431

§ 371 Date: Nov. 14, 2025

(30) Foreign Application Priority Data
Jun. 1, 2023 (GR) 20230100442
Feb. 5, 2024 (GR) 20240100072

(51) Int. Cl. C07D 487/04; A61K 31/437; A61K 31/5025; A61K 31/519; A61K 31/53; C07B 59/00; C07D 471/04; C07D 519/00

(52) U.S. Cl. CPC C07D 487/04; A61K 31/437; A61K 31/5025; A61K 31/519; A61K 31/53; C07B 59/002; C07D 471/04; C07D 519/00; C07B 2200/05

(57) ABSTRACT

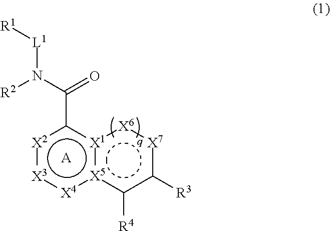

Disclosed are novel GPR68 modulators according to Formula (I), their pharmaceutically acceptable salts, and pharmaceutical compositions thereof. Also disclosed are methods of using such compounds and compositions to treat various diseases including cancer and proliferative diseases and disorders.

### TECHNICAL FIELD

The present invention relates to novel GPR68 modulators, their pharmaceutically acceptable salts, and pharmaceutical compositions thereof. The present invention also relates to methods of using such compounds and compositions, including to treat various diseases and disorders; particularly cancer and/or proliferative diseases or disorders.

### BACKGROUND

GPR68 (also known as ovarian cancer G protein-coupled receptor 1 or OGR1) is a proton-sensing G-protein-coupled receptor (GPCR) that responds to extracellular acidity and regulates a variety of cellular functions. It has been found that GPR68 expression is highly upregulated in numerous types of cancer, such as colon cancer, medulloblastoma, head and neck cancer, pancreatic cancer and prostate cancer, and it has been suggested that GPR68 has key roles in tumour biology. As such, GPR68 is an attractive target for anti-cancer drug development. Until now, however, few compounds have been identified as selective GPR68 ligands/modulators.

An unmet medical need therefore exists for effective and safe GPR68 modulators.

The present invention has been devised to address at least one of the challenges described above.

### SUMMARY OF THE INVENTION

Generally, provided herein are compounds and pharmaceutical compositions capable of modulating GPR68. Also provided are method of treatment and therapeutic/medical uses involving compounds or pharmaceutical compositions of this disclosure for for treating diseases, disorders or conditions associated with GPR68.

In an aspect of this disclosure, there is provided a compound of formula (1):

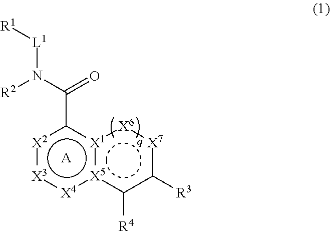

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein:
Q is O or S;
X¹ is selected from C and N;
X² is selected from CR⁵ and N;
X³ is selected from CR⁶ and N;
X⁴ is selected from CR⁷ and N;
X⁵ is selected from C and N;
wherein X¹, X², X³, X⁴ and X⁵ are selected so that the ring labelled A is aromatic;
X⁶ is selected from CR⁸R⁹, O and N;
X⁷ is selected from CR¹⁰, CR¹¹R¹², O and N;
q is 0 or 1;
wherein when q is 0, X⁷ is O or N; and when X⁷ is O or N and q is 1, X⁶ is CR⁸R⁹;
L¹ is a bond, C₁-C₆ alkylene, C₁-C₆ haloalkylene, C₃-C₆ cycloalkylene, or a 5- or 6-membered heterocycloalkylene having one or more heteroatoms selected from N, O and S;
R¹ is selected from C₁-C₄ alkyl, C₃-C₆ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, C₉-C₁₀ bicyclic aryl, 5- or 6-membered heteroaryl having one or more ring members selected from N, O and S, 8- to 10-membered bicyclic heteroaryl having one or more ring members selected from N, O, S and SO₂, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more ring members selected from N, O, S and SO₂, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is selected from H, C₁-C₄ alkyl and C₃-C₈ cycloakyl;
or R¹, R² and L¹ and the nitrogen atom to which R² and L¹ are attached form a 3- to 8-membered heterocycloalkyl having one or more heteroatoms selected from N, O and S, 8- to 11-membered bicyclic heterocycloalkyl having one or more heteroatoms selected from N, O and S, 5- or 6-membered heteroaryl having one or more ring members selected from N, O, S and SO₂, or 8- to 10-membered bicyclic heteroaryl having one or more heteroatoms selected from N, O and S, wherein said heterocycloalkyl, bicyclic heterocycloalkyl, heteroaryl and bicyclic heteroaryl are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy and C₁-C₄ alkyl-NR^{1a}R^{1b};
R³ is selected from H, OH, CN, NR^{3a}R^{3b}, C₁-C₄ alkyl, C₃-C₈ cycloalkyl, 3- to 8-membered heterocycloalkyl having one or more heteroatoms selected from N, O and S, phenyl, and 5- or 6-membered heteroaryl having one or more ring members selected from N, O, S and SO₂, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, CN, C₁-C₄ alkyl, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl;
R⁴ is selected from C(O)OH, C(O)NR^{4a}R^{4b}, C(S)NR^{4a}R^{4b} and 5- or 6-membered heteroaryl having one or more ring members selected from N, O, S and SO₂;
R^{4a} and R^{4b} are each independently selected from H and C₁-C₄ alkyl;
R⁵ and R⁶ are each independently selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl and 5- or 6-membered heteroaryl having one or more ring members selected from N, O, S and SO₂; or
R⁵ and R⁶ together with the carbon atoms to which they are attached form a 5- or 6-membered heterocycloalkyl having one or more heteroatoms selected from N, O and S; and
R⁷, R⁸, R⁹, R¹⁰, R¹¹, and R¹² are each independently selected from H, halogen, OH, CN, C₁-C₄ alkyl, C₁-C₄ haloalkyl, C₁-C₄ alkoxy, C₃-C₈ cycloalkyl, 3- to 8-membered heterocycloalkyl having one or more heteroatoms selected from N, O and S, phenyl and 5- or 6-membered heteroaryl having one or more ring members selected from N, O, S and SO₂.

In embodiments,
L¹ is a bond, C₁-C₆ alkylene, C₁-C₆ haloalkylene, C₃-C₆ cycloalkylene, or a 5- or 6-membered heterocycloalkylene having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, C₉-C₁₀ bicyclic aryl, 5- or 6-membered heteroaryl having one or more heteroatoms selected from N, O and S, 8- to 10-membered bicyclic heteroaryl having one or more ring members selected from N, O, S and SO₂, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is selected from H, C₁-C₄ alkyl and C₃-C₈ cycloakyl;
R³ is selected from H, OH, CN, NR^{3a}R^{3b}, C₁-C₄ alkyl, C₃-C₈ cycloalkyl, 3- to 8-membered heterocycloalkyl having one or more heteroatoms selected from N, O and S, phenyl, and 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, CN, C₁-C₄ alkyl, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl;
R⁴ is selected from C(O)OH, C(O)NR^{4a}R^{4b}, C(S)NR^{4a}R^{4b} and 5- or 6-membered heteroaryl having one or more ring members selected from N, O, S and SO₂;
R^{4a} and R^{4b} are each independently selected from H and C₁-C₄ alkyl;
R⁵ and R⁶ are each independently selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S; or
R⁵ and R⁶ together with the carbon atoms to which they are attached form a 5- or 6-membered heterocycloalkyl having one or more heteroatoms selected from N, O and S; and
R⁷, R⁸, R⁹, R¹⁰, R¹¹, and R¹² are each independently selected from H, halogen, OH, CN, C₁-C₄ alkyl, C₁-C₄ haloalkyl, C₁-C₄ alkoxy, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S.

In embodiments,
L¹ is a bond, C₁-C₆ alkylene, C₁-C₆ haloalkylene, C₃-C₆ cycloalkylene, or a 5- or 6-membered heterocycloalkylene having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, 8- to 10-membered bicyclic heteroaryl having one or more ring members selected from N, O, S and SO₂, and phenoxy, wherein said alkyl, cycloalkyl, heterocycloalkyl, bicyclic cycloalkyl, bicyclic heterocycloalkyl, phenyl, heteroaryl, bicyclic heteroaryl, and phenoxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, 5- or 6-membered heteroaryl having one or more heteroatoms selected from N, O and S, benzyl, halophenoxy, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl; R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, C₁-C₄ alkyl, and NR^{3c}R^{3d};
R^{3a} is selected from H, C₁-C₄ alkyl, and C₁-C₄ alkyl-NR^{3e}R^{3f};
R^{3b} is selected from H and C₁-C₄ alkyl;
R^{3c} is selected from H and C₁-C₄ alkyl;
R^{3d} is selected from C₁-C₄ alkyl;
R^{3e} and R^{3f} are each independently selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments,
L¹ is a bond, C₁-C₆ alkylene, C₁-C₆ haloalkylene, or C₃-C₆ cycloalkylene;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, 8- to 10-membered bicyclic heteroaryl having one or more ring members selected from N, O, S and SO₂, and phenoxy, wherein said alkyl, cycloalkyl, heterocycloalkyl, bicyclic cycloalkyl, bicyclic heterocycloalkyl, phenyl, heteroaryl, bicyclic heteroaryl, and phenoxy are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 5- or 6-membered heteroaryl having one or more heteroatoms selected from N, O and S, benzyl, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH and NR^{3c}R^{3d};
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments,
L¹ is a bond, C₁-C₆ alkylene, or C₁-C₆ haloalkylene;
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, and 5- or 6-membered heteroaryl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH and NR^{3c}R^{3d};
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from the following groups:

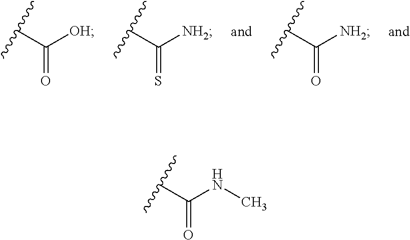

wherein [symbol] indicates the point of attachment to the rest of the compound;
R⁵ is selected from H and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments,
L¹ is a bond, C₁-C₆ alkylene, or C₃-C₆ cycloalkylene;
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, and 8- to 10-membered bicyclic heteroaryl having one or more ring members selected from N, O, S and SO₂, wherein said cycloalkyl, heterocycloalkyl, bicyclic cycloalkyl, bicyclic heterocycloalkyl, phenyl, heteroaryl, and bicyclic heteroaryl are each optionally substituted by one or more substituents independently selected from halogen, OH, C₁-C₄ alkyl, C₁-C₄ haloalkyl, C₁-C₄ alkoxy, and C₁-C₄ haloalkoxy;
R² is H;
R³ is selected from H, CN, NR^{3a}R^{3b}, C₁-C₄ alkyl optionally substituted by OH, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₃-C₈ cycloalkyl-C₁-C₄ alkoxy, C₁-C₄ haloalkoxy-C₁-C₄ alkyl, C₁-C₄ alkyl-S—C₁-C₄ alkyl, and C₁-C₄ alkyl-SO₂-C₁-C₄ alkyl;
R^{3a} is H;
R^{3b} is selected from C₁-C₄ alkyl;
R⁴ is selected from the following groups:

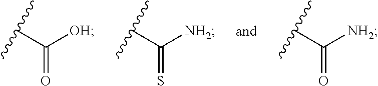

wherein [symbol] indicates the point of attachment to the rest of the compound;
R⁵ is H;
R⁶ is selected from H and C₁-C₄ alkyl; and
R⁷, R⁸, R⁹, R¹⁰, R¹¹, and R¹² are each H.

In embodiments,
L¹ is either a bond or C₁-C₆ alkylene;
R¹ is selected from C₃-C₈ cycloalkyl, C₈-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, OH, C₁-C₄ alkyl, C₁-C₄ haloalkyl, C₁-C₄ alkoxy, and C₁-C₄ haloalkoxy;
R² is H;
R³ is selected from NR^{3a}R^{3b}, C₁-C₄ alkyl optionally substituted by one or more substituents independently selected from OH and NR^{3c}R^{3d}, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₃-C₈ cycloalkoxy-C₁-C₄ alkyl, C₁-C₄ alkoxy-C₁-C₄ alkyl, C₁-C₄ haloalkoxy-C₁-C₄ alkyl, C₃-C₈ cycloalkyl-C₁-C₄ alkoxy, and alkyl-S—C₁-C₄ alkyl;
R^{3a} is H;
R^{3b} is selected from C₁-C₄ alkyl;
R⁴ is selected from the following groups:

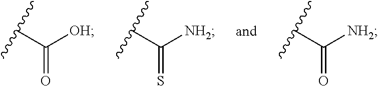

wherein [symbol] indicates the point of attachment to the rest of the compound;
R⁵, R⁷, R⁸, R⁹, R¹⁰, R¹¹, and R¹² are each H; and
R⁶ is selected from H and C₁-C₄ alkyl.

In embodiments, Q is O.

In embodiments, L is either a bond or methylene.

In embodiments, R¹ is selected from:
C₃-C₈ cycloalkyl optionally substituted by one or more substituents independently selected from halogen and C₁-C₄ haloalkoxy;
C₈-C₁₁ bicyclic cycloalkyl optionally substituted by one or more substituents independently selected from halogen, OH, C₁-C₄ alkyl and C₁-C₄ alkoxy;
3- to 8-membered heterocycloalkyl having one or more ring members selected from N, O, S and SO₂;
8- to 11-membered bicyclic heterocycloalkyl having one or more ring members selected from N, O, S and SO₂, optionally substituted by one or more substituents independently selected from halogen;
phenyl optionally substituted by one or more substituents independently selected from halogen and C₁-C₄ alkyl;
5- or 6-membered heteroaryl having one or more heteroatoms selected from N, O and S, optionally substituted by one or more substituents independently selected from C₁-C₄ alkyl and C₁-C₄ haloalkyl; and
8- to 10-membered bicyclic heteroaryl having one or more ring members selected from N, O, S and SO₂, optionally substituted substituted by one or more substituents independently selected from halogen and C₁-C₄ alkyl.

In embodiments, R² is H.

In embodiments, R³ is selected from:
NR^{3a}R^{3b};
C₁-C₄ alkyl optionally substituted by OH;
C₃-C₈ cycloalkyl;
3- to 8-membered heterocycloalkyl having one or more heteroatoms selected from N, O and S;
5- or 6-membered heteroaryl having one or more heteroatoms selected from N, O and S;
C₁-C₄ alkoxy-C₁-C₄ alkyl;
C₃-C₈ cycloalkyl-C₁-C₄ alkoxy;
C₃-C₈ cycloalkoxy-C₁-C₄ alkyl;
C₁-C₄ haloalkoxy-C₁-C₄ alkyl; and
C₁-C₄ alkyl-S—C₁-C₄ alkyl;
   wherein

### R^{3a} is H; and

R^{3b} is selected from C₁-C₄ alkyl.

In embodiments, R³ is selected from C₁-C₄ alkoxy-C₁-C₄ alkyl.

In embodiments, R⁴ is selected from the following groups:

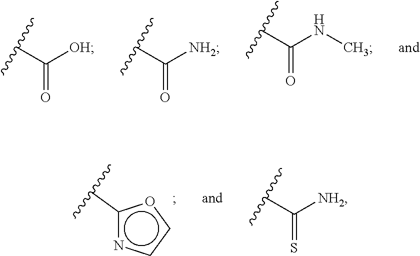

wherein [symbol] indicates the point of attachment to the rest of the compound.

In embodiments, R⁵ is H.

In embodiments, R⁶ is H or methyl.

In embodiments, R⁷, R⁸, R⁹, R¹⁰, R¹¹, and R¹² are each H.

In an aspect of this disclosure, there is provided a compound of formula (2a):

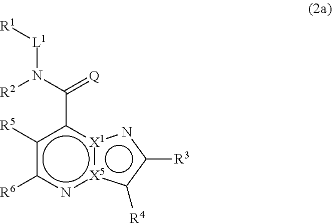

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein
Q is O or S;
X¹ is selected from C and N;
X² is selected from CR⁵ and N;
X³ is selected from CR⁶ and N;
X⁴ is selected from CR⁷ and N;
X⁵ is selected from C and N;
wherein X¹, X², X³, X⁴ and X⁵ are selected so that the ring labelled A is aromatic;
X⁶ is selected from CR⁸R⁹, O and N;
X⁷ is selected from CR¹⁰, CR¹¹R¹², O and N;
q is 0 or 1;
wherein when q is 0, X⁷ is O or N; and when X⁷ is O or N and q is 1, X⁶ is CR⁸R⁹;
L¹ is a bond, C₁-C₆ alkylene, C₁-C₆ haloalkylene, C₃-C₆ cycloalkylene, or a 5- or 6-membered heterocycloalkylene having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, C₉-C₁₀ bicyclic aryl, 5- or 6-membered heteroaryl having one or more heteroatoms selected from N, O and S, 8- to 10-membered bicyclic heteroaryl having one or more ring members selected from N, O, S and SO₂, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is selected from H, C₁-C₄ alkyl and C₃-C₈ cycloakyl;
or R¹, R² and L¹ and the nitrogen atom to which R² and L¹ are attached form a 3- to 8-membered heterocycloalkyl having one or more heteroatoms selected from N, O and S, 8- to 11-membered bicyclic heterocycloalkyl having one or more heteroatoms selected from N, O and S, 5- or 6-membered heteroaryl having one or more heteroatoms selected from N, O and S, or 8- to 10-membered bicyclic heteroaryl having one or more heteroatoms selected from N, O and S, wherein said heterocycloalkyl, bicyclic heterocycloalkyl, heteroaryl and bicyclic heteroaryl are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy and C₁-C₄ alkyl-NR^{1a}R^{1b};
R³ is selected from H, OH, CN, NR^{3a}R^{3b}, C₁-C₄ alkyl, C₃-C₈ cycloalkyl, 3- to 8-membered heterocycloalkyl having one or more heteroatoms selected from N, O and S, phenyl, and 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, CN, C₁-C₄ alkyl, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} and R^{4b} are each independently selected from H and C₁-C₄ alkyl;
R⁵ and R⁶ are each independently selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S; or
R⁵ and R⁶ together with the carbon atoms to which they are attached form a 5- or 6-membered heterocycloalkyl having one or more heteroatoms selected from N, O and S; and
R⁷, R⁸, R⁹, R¹⁰, R¹¹, and R¹² are each independently selected from H, halogen, OH, CN, C₁-C₄ alkyl, C₁-C₄ haloalkyl, C₁-C₄ alkoxy, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S.

In embodiments,
L¹ is a bond, C₁-C₆ alkylene, C₁-C₆ haloalkylene, C₃-C₆ cycloalkylene, or a 5- or 6-membered heterocycloalkylene having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, C₉-C₁₀ bicyclic aryl, 5- or 6-membered heteroaryl having one or more heteroatoms selected from N, O and S, 8- to 10-membered bicyclic heteroaryl having one or more ring members selected from N, O, S and SO₂, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is selected from H, C₁-C₄ alkyl and C₃-C₈ cycloakyl;
R³ is selected from H, OH, CN, NR^{3a}R^{3b}, C₁-C₄ alkyl, C₃-C₈ cycloalkyl, 3- to 8-membered heterocycloalkyl having one or more heteroatoms selected from N, O and S, phenyl, and 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, CN, C₁-C₄ alkyl, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} and R^{4b} are each independently selected from H and C₁-C₄ alkyl;
R⁵ and R⁶ are each independently selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S; or
R⁵ and R⁶ together with the carbon atoms to which they are attached form a 5- or 6-membered heterocycloalkyl having one or more heteroatoms selected from N, O and S; and
R⁷, R⁸, R⁹, R¹⁰, R¹¹, and R¹² are each independently selected from H, halogen, OH, CN, C₁-C₄ alkyl, C₁-C₄ haloalkyl, C₁-C₄ alkoxy, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S.

In embodiments,
L¹ is a bond, C₁-C₆ alkylene, C₁-C₆ haloalkylene, C₃-C₆ cycloalkylene, or a 5- or 6-membered heterocycloalkylene having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more S and SO₂.selected from N, O, S and SO₂, 8- to 10-membered bicyclic heteroaryl having one or more ring members selected from N, O, S and SO₂, and phenoxy, wherein said alkyl, cycloalkyl, heterocycloalkyl, bicyclic cycloalkyl, bicyclic heterocycloalkyl, phenyl, heteroaryl, bicyclic heteroaryl, and phenoxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, 5- or 6-membered heteroaryl having one or more heteroatoms selected from N, O and S, benzyl, halophenoxy, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, C₁-C₄ alkyl, and NR^{3c}R^{3d};
R^{3a} is selected from H, C₁-C₄ alkyl, and C₁-C₄ alkyl-NR^{3e}R^{3f};
R^{3b} is selected from H and C₁-C₄ alkyl;
R^{3c} is selected from H and C₁-C₄ alkyl;
R^{3d} is selected from C₁-C₄ alkyl;
R^{3e} and R^{3f} are each independently selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments,
L¹ is a bond, C₁-C₆ alkylene, C₁-C₆ haloalkylene, or C₃-C₆ cycloalkylene;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, 8- to 10-membered bicyclic heteroaryl having one or more ring members selected from N, O, S and SO₂, and phenoxy, wherein said alkyl, cycloalkyl, heterocycloalkyl, bicyclic cycloalkyl, bicyclic heterocycloalkyl, phenyl, heteroaryl, bicyclic heteroaryl, and phenoxy are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 5- or 6-membered heteroaryl having one or more heteroatoms selected from N, O and S, benzyl, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH and NR^{3c}R^{3d};
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments,
L¹ is a bond, C₁-C₆ alkylene, or C₁-C₆ haloalkylene;
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, and 5- or 6-membered heteroaryl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH and NR^{3c}R^{3d};
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from the following groups:

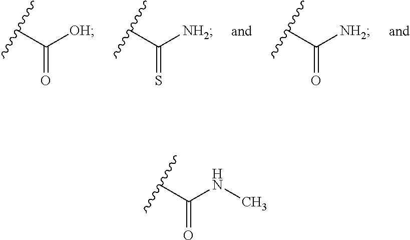

wherein [symbol] indicates the point of attachment to the rest of the compound;
R⁵ is selected from H and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments,
L¹ is a bond, C₁-C₆ alkylene, or C₃-C₆ cycloalkylene;
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, and 8- to 10-membered bicyclic heteroaryl having one or more ring members selected from N, O, S and SO₂, wherein said cycloalkyl, heterocycloalkyl, bicyclic cycloalkyl, bicyclic heterocycloalkyl, phenyl, heteroaryl, and bicyclic heteroaryl are each optionally substituted by one or more substituents independently selected from halogen, OH, C₁-C₄ alkyl, C₁-C₄ haloalkyl, C₁-C₄ alkoxy, and C₁-C₄ haloalkoxy;
R² is H;
R³ is selected from H, CN, NR^{3a}R^{3b}, C₁-C₄ alkyl optionally substituted by OH, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₃-C₈ cycloalkyl-C₁-C₄ alkoxy, C₁-C₄ haloalkoxy-C₁-C₄ alkyl, C₁-C₄ alkyl-S—C₁-C₄ alkyl, and C₁-C₄ alkyl-SO₂-C₁-C₄ alkyl;
R^{3a} is H;
R^{3b} is selected from C₁-C₄ alkyl;
R⁴ is selected from the following groups:

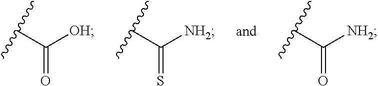

wherein [symbol] indicates the point of attachment to the rest of the compound;
R⁵ is H;
R⁶ is selected from H and C₁-C₄ alkyl; and
R⁷, R⁸, R⁹, R¹⁰, R¹¹, and R¹² are each H.

In embodiments,
L¹ is either a bond or C₁-C₆ alkylene;
R¹ is selected from C₃-C₈ cycloalkyl, C₈-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, OH, C₁-C₄ alkyl, C₁-C₄ haloalkyl, C₁-C₄ alkoxy, and C₁-C₄ haloalkoxy;
R² is H;
R³ is selected from NR^{3a}R^{3b}, C₁-C₄ alkyl optionally substituted by one or more substituents independently selected from OH and NR^{3c}R^{3d}, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₃-C₈ cycloalkoxy-C₁-C₄ alkyl, C₁-C₄ alkoxy-C₁-C₄ alkyl, C₁-C₄ haloalkoxy-C₁-C₄ alkyl, C₃-C₈ cycloalkyl-C₁-C₄ alkoxy, and alkyl-S—C₁-C₄ alkyl;
R^{3a} is H;
R^{3b} is selected from C₁-C₄ alkyl;
R⁴ is selected from the following groups:

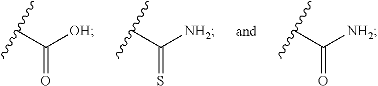

wherein [symbol] indicates the point of attachment to the rest of the compound R⁵, R⁷, R⁸, R⁹, R¹⁰, R¹¹, and R¹² are each H; and
R⁶ is selected from H and C₁-C₄ alkyl.

In embodiments, Q is O.

In embodiments, L is either a bond or methylene.

In embodiments, R¹ is selected from:
C₃-C₈ cycloalkyl optionally substituted by one or more substituents independently selected from halogen and C₁-C₄ haloalkoxy;
C₈-C₁₁ bicyclic cycloalkyl optionally substituted by one or more substituents independently selected from halogen, OH, C₁-C₄ alkyl and C₁-C₄ alkoxy;
3- to 8-membered heterocycloalkyl having one or more ring members selected from N, O, S and SO₂;
8- to 11-membered bicyclic heterocycloalkyl having one or more ring members selected from N, O, S and SO₂, optionally substituted by one or more substituents independently selected from halogen;
phenyl optionally substituted by one or more substituents independently selected from halogen and C₁-C₄ alkyl;
5- or 6-membered heteroaryl having one or more heteroatoms selected from N, O and S, optionally substituted by one or more substituents independently selected from C₁-C₄ alkyl and C₁-C₄ haloalkyl; and
8- to 10-membered bicyclic heteroaryl having one or more ring members selected from N, O, S and SO₂, optionally substituted substituted by one or more substituents independently selected from halogen and C₁-C₄ alkyl.

In embodiments, R² is H.

In embodiments, R³ is selected from:
NR^{3a}R^{3b};
C₁-C₄ alkyl optionally substituted by OH;
C₃-C₈ cycloalkyl;
3- to 8-membered heterocycloalkyl having one or more heteroatoms selected from N, O and S;
5- or 6-membered heteroaryl having one or more heteroatoms selected from N, O and S;
C₁-C₄ alkoxy-C₁-C₄ alkyl;
C₃-C₈ cycloalkyl-C₁-C₄ alkoxy;
C₃-C₈ cycloalkoxy-C₁-C₄ alkyl;
C₁-C₄ haloalkoxy-C₁-C₄ alkyl; and
C₁-C₄ alkyl-S—C₁-C₄ alkyl;
   wherein

### R^{3a} is H; and

R^{3b} is selected from C₁-C₄ alkyl.

In embodiments, R³ is selected from C₁-C₄ alkoxy-C₁-C₄ alkyl.

In embodiments, R⁴ is selected from the following groups:

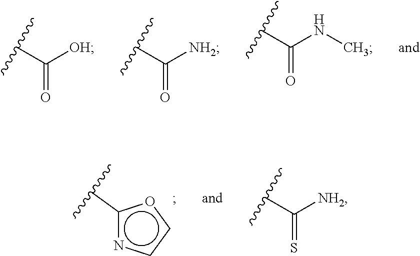

wherein [symbol] indicates the point of attachment to the rest of the compound.

In embodiments, R⁵ is H.

In embodiments, R⁶ is H or methyl.

In embodiments, R⁷, R⁸, R⁹, R¹⁰, R¹¹, and R¹² are each H.

In an aspect of this disclosure, there is provided a compound of formula (2aa):

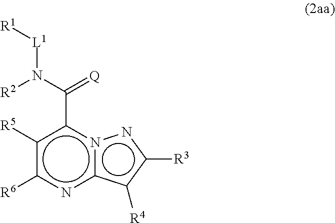

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein
Q is O or S;
X¹ is selected from C and N;
X² is selected from CR⁵ and N;
X³ is selected from CR⁶ and N;
X⁴ is selected from CR⁷ and N;
X⁵ is selected from C and N;
wherein X¹, X², X³, X⁴ and X⁵ are selected so that the ring labelled A is aromatic;
X⁶ is selected from CR⁸R⁹, O and N;
X⁷ is selected from CR¹⁰, CR¹¹R¹², O and N;
q is 0 or 1;
wherein when q is 0, X⁷ is O or N; and when X⁷ is O or N and q is 1, X⁶ is CR⁸R⁹;
L¹ is a bond, C₁-C₆ alkylene, C₁-C₆ haloalkylene, C₃-C₆ cycloalkylene, or a 5- or 6-membered heterocycloalkylene having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, C₉-C₁₀ bicyclic aryl, 5- or 6-membered heteroaryl having one or more heteroatoms selected from N, O and S, 8- to 10-membered bicyclic heteroaryl having one or more ring members selected from N, O, S and SO₂, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is selected from H, C₁-C₄ alkyl and C₃-C₈ cycloakyl;
or R¹, R² and L¹ and the nitrogen atom to which R² and L¹ are attached form a 3- to 8-membered heterocycloalkyl having one or more heteroatoms selected from N, O and S, 8- to 11-membered bicyclic heterocycloalkyl having one or more heteroatoms selected from N, O and S, 5- or 6-membered heteroaryl having one or more heteroatoms selected from N, O and S, or 8- to 10-membered bicyclic heteroaryl having one or more heteroatoms selected from N, O and S, wherein said heterocycloalkyl, bicyclic heterocycloalkyl, heteroaryl and bicyclic heteroaryl are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy and C₁-C₄ alkyl-NR^{1a}R^{1b};
R³ is selected from H, OH, CN, NR^{3a}R^{3b}, C₁-C₄ alkyl, C₃-C₈ cycloalkyl, 3- to 8-membered heterocycloalkyl having one or more heteroatoms selected from N, O and S, phenyl, and 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, CN, C₁-C₄ alkyl, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} and R^{4b} are each independently selected from H and C₁-C₄ alkyl;
R⁵ and R⁶ are each independently selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S; or
R⁵ and R⁶ together with the carbon atoms to which they are attached form a 5- or 6-membered heterocycloalkyl having one or more heteroatoms selected from N, O and S; and
R⁷, R⁸, R⁹, R¹⁰, R¹¹, and R¹² are each independently selected from H, halogen, OH, CN, C₁-C₄ alkyl, C₁-C₄ haloalkyl, C₁-C₄ alkoxy, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S.

In embodiments,
L¹ is a bond, C₁-C₆ alkylene, C₁-C₆ haloalkylene, C₃-C₆ cycloalkylene, or a 5- or 6-membered heterocycloalkylene having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, C₉-C₁₀ bicyclic aryl, 5- or 6-membered heteroaryl having one or more heteroatoms selected from N, O and S, 8- to 10-membered bicyclic heteroaryl having one or more ring members selected from N, O, S and SO₂, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is selected from H, C₁-C₄ alkyl and C₃-C₈ cycloakyl;
R³ is selected from H, OH, CN, NR^{3a}R^{3b}, C₁-C₄ alkyl, C₃-C₈ cycloalkyl, 3- to 8-membered heterocycloalkyl having one or more heteroatoms selected from N, O and S, phenyl, and 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, CN, C₁-C₄ alkyl, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} and R^{4b} are each independently selected from H and C₁-C₄ alkyl;
R⁵ and R⁶ are each independently selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S; or
R⁵ and R⁶ together with the carbon atoms to which they are attached form a 5- or 6-membered heterocycloalkyl having one or more heteroatoms selected from N, O and S; and
R⁷, R⁸, R⁹, R¹⁰, R¹¹, and R¹² are each independently selected from H, halogen, OH, CN, C₁-C₄ alkyl, C₁-C₄ haloalkyl, C₁-C₄ alkoxy, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S.

In embodiments,
L¹ is a bond, C₁-C₆ alkylene, C₁-C₆ haloalkylene, C₃-C₆ cycloalkylene, or a 5- or 6-membered heterocycloalkylene having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, 8- to 10-membered bicyclic heteroaryl having one or more ring members selected from N, O, S and SO₂, and phenoxy, wherein said alkyl, cycloalkyl, heterocycloalkyl, bicyclic cycloalkyl, bicyclic heterocycloalkyl, phenyl, heteroaryl, bicyclic heteroaryl, and phenoxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, 5- or 6-membered heteroaryl having one or more heteroatoms selected from N, O and S, benzyl, halophenoxy, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl; R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, C₁-C₄ alkyl, and NR^{3c}R^{3d};
R^{3a} is selected from H, C₁-C₄ alkyl, and C₁-C₄ alkyl-NR^{3e}R^{3f};
R^{3b} is selected from H and C₁-C₄ alkyl;
R^{3c} is selected from H and C₁-C₄ alkyl;
R^{3d} is selected from C₁-C₄ alkyl;
R^{3e} and R^{3f} are each independently selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments,
L¹ is a bond, C₁-C₆ alkylene, C₁-C₆ haloalkylene, or C₃-C₆ cycloalkylene;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, 8- to 10-membered bicyclic heteroaryl having one or more ring members selected from N, O, S and SO₂, and phenoxy, wherein said alkyl, cycloalkyl, heterocycloalkyl, bicyclic cycloalkyl, bicyclic heterocycloalkyl, phenyl, heteroaryl, bicyclic heteroaryl, and phenoxy are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 5- or 6-membered heteroaryl having one or more heteroatoms selected from N, O and S, benzyl, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH and NR^{3c}R^{3d};
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments,
L¹ is a bond, C₁-C₆ alkylene, or C₁-C₆ haloalkylene;
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, and 5- or 6-membered heteroaryl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH and NR^{3c}R^{3d};
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from the following groups:

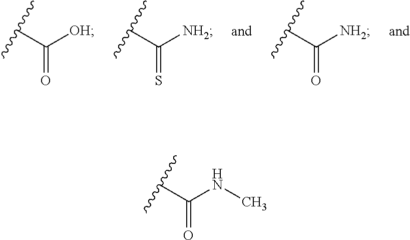

wherein [symbol] indicates the point of attachment to the rest of the compound; R⁵ is selected from H and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments,
L¹ is a bond, C₁-C₆ alkylene, or C₃-C₆ cycloalkylene;
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, and 8- to 10-membered bicyclic heteroaryl having one or more ring members selected from N, O, S and SO₂, wherein said cycloalkyl, heterocycloalkyl, bicyclic cycloalkyl, bicyclic heterocycloalkyl, phenyl, heteroaryl, and bicyclic heteroaryl are each optionally substituted by one or more substituents independently selected from halogen, OH, C₁-C₄ alkyl, C₁-C₄ haloalkyl, C₁-C₄ alkoxy, and C₁-C₄ haloalkoxy;
R² is H;
R³ is selected from H, CN, NR^{3a}R^{3b}, C₁-C₄ alkyl optionally substituted by OH, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₃-C₈ cycloalkyl-C₁-C₄ alkoxy, C₁-C₄ haloalkoxy-C₁-C₄ alkyl, C₁-C₄ alkyl-S—C₁-C₄ alkyl, and C₁-C₄ alkyl-SO₂-C₁-C₄ alkyl;
R^{3a} is H;
R^{3b} is selected from C₁-C₄ alkyl;
R⁴ is selected from the following groups:

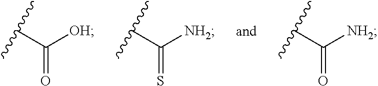

wherein [symbol] indicates the point of attachment to the rest of the compound;
R⁵ is H;
R⁶ is selected from H and C₁-C₄ alkyl; and
R⁷, R⁸, R⁹, R¹⁰, R¹¹, and R¹² are each H.

In embodiments,
L¹ is either a bond or C₁-C₆ alkylene;
R¹ is selected from C₃-C₈ cycloalkyl, C₈-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, OH, C₁-C₄ alkyl, C₁-C₄ haloalkyl, C₁-C₄ alkoxy, and C₁-C₄ haloalkoxy;
R² is H;
R³ is selected from NR^{3a}R^{3b}, C₁-C₄ alkyl optionally substituted by one or more substituents independently selected from OH and NR^{3c}R^{3d}, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₃-C₈ cycloalkoxy-C₁-C₄ alkyl, C₁-C₄ alkoxy-C₁-C₄ alkyl, C₁-C₄ haloalkoxy-C₁-C₄ alkyl, C₃-C₈ cycloalkyl-C₁-C₄ alkoxy, and alkyl-S—C₁-C₄ alkyl;
R^{3a} is H;
R^{3b} is selected from C₁-C₄ alkyl;
R⁴ is selected from the following groups:

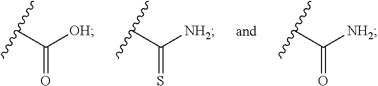

wherein [symbol] indicates the point of attachment to the rest of the compound R⁵, R⁷, R⁸, R⁹, R¹⁰, R¹¹, and R¹² are each H; and
R⁶ is selected from H and C₁-C₄ alkyl.

In embodiments, Q is O.

In embodiments, L is either a bond or methylene.

In embodiments, R¹ is selected from:
C₃-C₈ cycloalkyl optionally substituted by one or more substituents independently selected from halogen and C₁-C₄ haloalkoxy;
C₈-C₁₁ bicyclic cycloalkyl optionally substituted by one or more substituents independently selected from halogen, OH, C₁-C₄ alkyl and C₁-C₄ alkoxy;
3- to 8-membered heterocycloalkyl having one or more ring members selected from N, O, S and SO₂;
8- to 11-membered bicyclic heterocycloalkyl having one or more ring members selected from N, O, S and SO₂, optionally substituted by one or more substituents independently selected from halogen;
phenyl optionally substituted by one or more substituents independently selected from halogen and C₁-C₄ alkyl;
5- or 6-membered heteroaryl having one or more heteroatoms selected from N, O and S, optionally substituted by one or more substituents independently selected from C₁-C₄ alkyl and C₁-C₄ haloalkyl; and
8- to 10-membered bicyclic heteroaryl having one or more ring members selected from N, O, S and SO₂, optionally substituted substituted by one or more substituents independently selected from halogen and C₁-C₄ alkyl.

In embodiments, R² is H.

In embodiments, R³ is selected from:
NR^{3a}R^{3b};
C₁-C₄ alkyl optionally substituted by OH;
C₃-C₈ cycloalkyl;
3- to 8-membered heterocycloalkyl having one or more heteroatoms selected from N, O and S;
5- or 6-membered heteroaryl having one or more heteroatoms selected from N, O and S;
C₁-C₄ alkoxy-C₁-C₄ alkyl;
C₃-C₈ cycloalkyl-C₁-C₄ alkoxy;
C₃-C₈ cycloalkoxy-C₁-C₄ alkyl;
C₁-C₄ haloalkoxy-C₁-C₄ alkyl; and
C₁-C₄ alkyl-S—C₁-C₄ alkyl;
   wherein

### R^{3a} is H; and

R^{3b} is selected from C₁-C₄ alkyl.

In embodiments, R³ is selected from C₁-C₄ alkoxy-C₁-C₄ alkyl.

In embodiments, R⁴ is selected from the following groups:

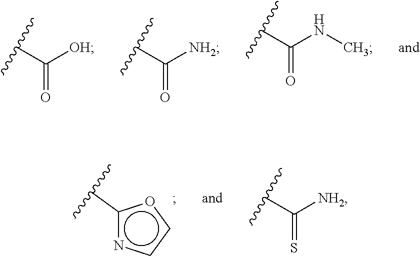

wherein [symbol] indicates the point of attachment to the rest of the compound.

In embodiments, R⁵ is H.

In embodiments, R⁶ is H or methyl.

In embodiments, R⁷, R⁸, R⁹, R¹⁰, R¹¹, and R¹² are each H.

In an aspect of this disclosure, there is provided a compound of formula (2ab):

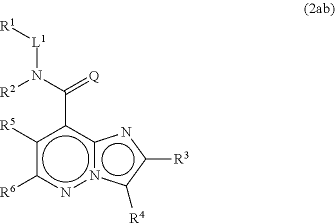

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein
Q is O or S;
X¹ is selected from C and N;
X² is selected from CR⁵ and N;
X³ is selected from CR⁶ and N;
X⁴ is selected from CR⁷ and N;
X⁵ is selected from C and N;
wherein X¹, X², X³, X⁴ and X⁵ are selected so that the ring labelled A is aromatic;
X⁶ is selected from CR⁸R⁹, O and N;
X⁷ is selected from CR¹⁰, CR¹¹R¹², O and N;
q is 0 or 1;
wherein when q is 0, X⁷ is O or N; and when X⁷ is O or N and q is 1, X⁶ is CR⁸R⁹;
L¹ is a bond, C₁-C₆ alkylene, C₁-C₆ haloalkylene, C₃-C₆ cycloalkylene, or a 5- or 6-membered heterocycloalkylene having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, C₉-C₁₀ bicyclic aryl, 5- or 6-membered heteroaryl having one or more heteroatoms selected from N, O and S, 8- to 10-membered bicyclic heteroaryl having one or more ring members selected from N, O, S and SO₂, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is selected from H, C₁-C₄ alkyl and C₃-C₈ cycloakyl;
or R¹, R² and L¹ and the nitrogen atom to which R² and L¹ are attached form a 3- to 8-membered heterocycloalkyl having one or more heteroatoms selected from N, O and S, 8- to 11-membered bicyclic heterocycloalkyl having one or more heteroatoms selected from N, O and S, 5- or 6-membered heteroaryl having one or more heteroatoms selected from N, O and S, or 8- to 10-membered bicyclic heteroaryl having one or more heteroatoms selected from N, O and S, wherein said heterocycloalkyl, bicyclic heterocycloalkyl, heteroaryl and bicyclic heteroaryl are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy and C₁-C₄ alkyl-NR^{1a}R^{1b};
R³ is selected from H, OH, CN, NR^{3a}R^{3b}, C₁-C₄ alkyl, C₃-C₈ cycloalkyl, 3- to 8-membered heterocycloalkyl having one or more heteroatoms selected from N, O and S, phenyl, and 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, CN, C₁-C₄ alkyl, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} and R^{4b} are each independently selected from H and C₁-C₄ alkyl;
R⁵ and R⁶ are each independently selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S; or
R⁵ and R⁶ together with the carbon atoms to which they are attached form a 5- or 6-membered heterocycloalkyl having one or more heteroatoms selected from N, O and S; and
R⁷, R⁸, R⁹, R¹⁰, R¹¹, and R¹² are each independently selected from H, halogen, OH, CN, C₁-C₄ alkyl, C₁-C₄ haloalkyl, C₁-C₄ alkoxy, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S.

In embodiments,
L¹ is a bond, C₁-C₆ alkylene, C₁-C₆ haloalkylene, C₃-C₆ cycloalkylene, or a 5- or 6-membered heterocycloalkylene having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, C₉-C₁₀ bicyclic aryl, 5- or 6-membered heteroaryl having one or more heteroatoms selected from N, O and S, 8- to 10-membered bicyclic heteroaryl having one or more ring members selected from N, O, S and SO₂, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is selected from H, C₁-C₄ alkyl and C₃-C₈ cycloakyl;
R³ is selected from H, OH, CN, NR^{3a}R^{3b}, C₁-C₄ alkyl, C₃-C₈ cycloalkyl, 3- to 8-membered heterocycloalkyl having one or more heteroatoms selected from N, O and S, phenyl, and 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, CN, C₁-C₄ alkyl, and NR^{3c}R^{3d};
R^{3a} is selected from H, C₁-C₄ alkyl, C₁-C₄ alkyl-OH, and C₁-C₄ alkyl-NR^{3e}R^{3f}, R^{3b}, R^{3c}, R^{3d}, R^{3e} and R^{3f} are each independently selected from H and C₁-C₄ alkyl; R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} and R^{4b} are each independently selected from H and C₁-C₄ alkyl;
R⁵ and R⁶ are each independently selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S; or
R⁵ and R⁶ together with the carbon atoms to which they are attached form a 5- or 6-membered heterocycloalkyl having one or more heteroatoms selected from N, O and S; and R⁷, R⁸, R⁹, R¹⁰, R¹¹, and R¹² are each independently selected from H, halogen, OH, CN, C₁-C₄ alkyl, C₁-C₄ haloalkyl, C₁-C₄ alkoxy, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S.

In embodiments,
L¹ is a bond, C₁-C₆ alkylene, C₁-C₆ haloalkylene, C₃-C₆ cycloalkylene, or a 5- or 6-membered heterocycloalkylene having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, 8- to 10-membered bicyclic heteroaryl having one or more ring members selected from N, O, S and SO₂, and phenoxy, wherein said alkyl, cycloalkyl, heterocycloalkyl, bicyclic cycloalkyl, bicyclic heterocycloalkyl, phenyl, heteroaryl, bicyclic heteroaryl, and phenoxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, 5- or 6-membered heteroaryl having one or more heteroatoms selected from N, O and S, benzyl, halophenoxy, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, C₁-C₄ alkyl, and NR^{3c}R^{3d};
R^{3a} is selected from H, C₁-C₄ alkyl, and C₁-C₄ alkyl-NR^{3e}R^{3f};
R^{3b} is selected from H and C₁-C₄ alkyl;
R^{3c} is selected from H and C₁-C₄ alkyl;
R^{3d} is selected from C₁-C₄ alkyl;
R^{3e} and R^{3f} are each independently selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments,
L¹ is a bond, C₁-C₆ alkylene, C₁-C₆ haloalkylene, or C₃-C₆ cycloalkylene;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, 8- to 10-membered bicyclic heteroaryl having one or more ring members selected from N, O, S and SO₂, and phenoxy, wherein said alkyl, cycloalkyl, heterocycloalkyl, bicyclic cycloalkyl, bicyclic heterocycloalkyl, phenyl, heteroaryl, bicyclic heteroaryl, and phenoxy are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 5- or 6-membered heteroaryl having one or more heteroatoms selected from N, O and S, benzyl, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH and NR^{3c}R^{3d};
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments,
L¹ is a bond, C₁-C₆ alkylene, or C₁-C₆ haloalkylene;
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, and 5- or 6-membered heteroaryl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH and NR^{3c}R^{3d};
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from the following groups:

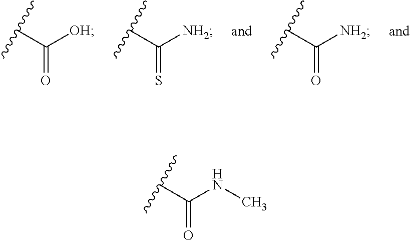

wherein [symbol] indicates the point of attachment to the rest of the compound;
R⁵ is selected from H and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments,
L¹ is a bond, C₁-C₆ alkylene, or C₃-C₆ cycloalkylene;
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, and 8- to 10-membered bicyclic heteroaryl having one or more ring members selected from N, O, S and SO₂, wherein said cycloalkyl, heterocycloalkyl, bicyclic cycloalkyl, bicyclic heterocycloalkyl, phenyl, heteroaryl, and bicyclic heteroaryl are each optionally substituted by one or more substituents independently selected from halogen, OH, C₁-C₄ alkyl, C₁-C₄ haloalkyl, C₁-C₄ alkoxy, and C₁-C₄ haloalkoxy;
R² is H;
R³ is selected from H, CN, NR^{3a}R^{3b}, C₁-C₄ alkyl optionally substituted by OH, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₃-C₈ cycloalkyl-C₁-C₄ alkoxy, C₁-C₄ haloalkoxy-C₁-C₄ alkyl, C₁-C₄ alkyl-S—C₁-C₄ alkyl, and C₁-C₄ alkyl-SO₂-C₁-C₄ alkyl;
R^{3a} is H;
R^{3b} is selected from C₁-C₄ alkyl;
R⁴ is selected from the following groups:

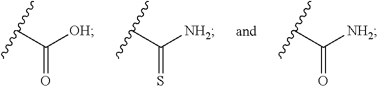

wherein [symbol] indicates the point of attachment to the rest of the compound;
R⁵ is H;
R⁶ is selected from H and C₁-C₄ alkyl; and
R⁷, R⁸, R⁹, R¹⁰, R¹¹, and R¹² are each H.

In embodiments,
L¹ is either a bond or C₁-C₆ alkylene;
R¹ is selected from C₃-C₈ cycloalkyl, C₈-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, OH, C₁-C₄ alkyl, C₁-C₄ haloalkyl, C₁-C₄ alkoxy, and C₁-C₄ haloalkoxy;
R² is H;
R³ is selected from NR^{3a}R^{3b}, C₁-C₄ alkyl optionally substituted by one or more substituents independently selected from OH and NR^{3c}R^{3d}, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₃-C₈ cycloalkoxy-C₁-C₄ alkyl, C₁-C₄ alkoxy-C₁-C₄ alkyl, C₁-C₄ haloalkoxy-C₁-C₄ alkyl, C₃-C₈ cycloalkyl-C₁-C₄ alkoxy, and alkyl-S—C₁-C₄ alkyl;
R^{3a} is H;
R^{3b} is selected from C₁-C₄ alkyl;
R⁴ is selected from the following groups:

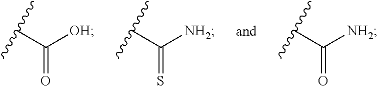

wherein [symbol] indicates the point of attachment to the rest of the compound R⁵, R⁷, R⁸, R⁹, R¹⁰, R¹¹, and R¹² are each H; and
R⁶ is selected from H and C₁-C₄ alkyl.

In embodiments, Q is O.

In embodiments, L is either a bond or methylene.

In embodiments, R¹ is selected from:
C₃-C₈ cycloalkyl optionally substituted by one or more substituents independently selected from halogen and C₁-C₄ haloalkoxy;
C₈-C₁₁ bicyclic cycloalkyl optionally substituted by one or more substituents independently selected from halogen, OH, C₁-C₄ alkyl and C₁-C₄ alkoxy;
3- to 8-membered heterocycloalkyl having one or more ring members selected from N, O, S and SO₂;
8- to 11-membered bicyclic heterocycloalkyl having one or more ring members selected from N, O, S and SO₂, optionally substituted by one or more substituents independently selected from halogen;
phenyl optionally substituted by one or more substituents independently selected from halogen and C₁-C₄ alkyl;
5- or 6-membered heteroaryl having one or more heteroatoms selected from N, O and S, optionally substituted by one or more substituents independently selected from C₁-C₄ alkyl and C₁-C₄ haloalkyl; and
8- to 10-membered bicyclic heteroaryl having one or more ring members selected from N, O, S and SO₂, optionally substituted substituted by one or more substituents independently selected from halogen and C₁-C₄ alkyl.

In embodiments, R² is H.

In embodiments, R³ is selected from:
NR^{3a}R^{3b};
C₁-C₄ alkyl optionally substituted by OH;
C₃-C₈ cycloalkyl;
3- to 8-membered heterocycloalkyl having one or more heteroatoms selected from N, O and S;
5- or 6-membered heteroaryl having one or more heteroatoms selected from N, O and S;
C₁-C₄ alkoxy-C₁-C₄ alkyl;
C₃-C₈ cycloalkyl-C₁-C₄ alkoxy;
C₃-C₈ cycloalkoxy-C₁-C₄ alkyl;
C₁-C₄ haloalkoxy-C₁-C₄ alkyl; and
C₁-C₄ alkyl-S—C₁-C₄ alkyl;
   wherein

### R^{3a} is H; and

R^{3b} is selected from C₁-C₄ alkyl.

In embodiments, R³ is selected from C₁-C₄ alkoxy-C₁-C₄ alkyl.

In embodiments, R⁴ is selected from the following groups:

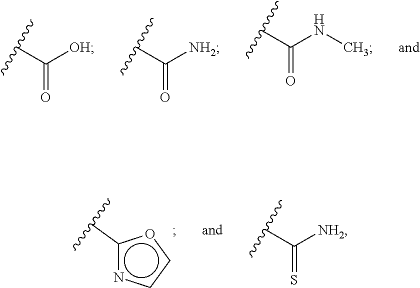

wherein [symbol] indicates the point of attachment to the rest of the compound.

In embodiments, R⁵ is H.

In embodiments, R⁶ is H or methyl.

In embodiments, R⁷, R⁸, R⁹, R¹⁰, R¹¹, and R¹² are each H.

In an aspect of this disclosure, there is provided a compound of formula (2ad):

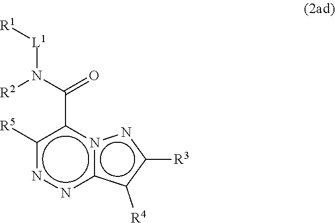

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein
Q is O or S;
X¹ is selected from C and N;
X² is selected from CR⁵ and N;
X³ is selected from CR⁶ and N;
X⁴ is selected from CR⁷ and N;
X⁵ is selected from C and N;
wherein X¹, X², X³, X⁴ and X⁵ are selected so that the ring labelled A is aromatic;
X⁶ is selected from CR⁸R⁹, O and N;
X⁷ is selected from CR¹⁰, CR¹¹R¹², O and N;
q is 0 or 1;
wherein when q is 0, X⁷ is O or N; and when X⁷ is O or N and q is 1, X⁶ is CR⁸R⁹;
L¹ is a bond, C₁-C₆ alkylene, C₁-C₆ haloalkylene, C₃-C₈ cycloalkylene, or a 5- or 6-membered heterocycloalkylene having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, C₉-C₁₀ bicyclic aryl, 5- or 6-membered heteroaryl having one or more heteroatoms selected from N, O and S, 8- to 10-membered bicyclic heteroaryl having one or more ring members selected from N, O, S and SO₂, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is selected from H, C₁-C₄ alkyl and C₃-C₈ cycloakyl;
or R¹, R² and L¹ and the nitrogen atom to which R² and L¹ are attached form a 3- to 8-membered heterocycloalkyl having one or more heteroatoms selected from N, O and S, 8- to 11-membered bicyclic heterocycloalkyl having one or more heteroatoms selected from N, O and S, 5- or 6-membered heteroaryl having one or more heteroatoms selected from N, O and S, or 8- to 10-membered bicyclic heteroaryl having one or more heteroatoms selected from N, O and S, wherein said heterocycloalkyl, bicyclic heterocycloalkyl, heteroaryl and bicyclic heteroaryl are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy and C₁-C₄ alkyl-NR^{1a}R^{1b};
R³ is selected from H, OH, CN, NR^{3a}R^{3b}, C₁-C₄ alkyl, C₃-C₈ cycloalkyl, 3- to 8-membered heterocycloalkyl having one or more heteroatoms selected from N, O and S, phenyl, and 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, CN, C₁-C₄ alkyl, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} and R^{4b} are each independently selected from H and C₁-C₄ alkyl;
R⁵ and R⁶ are each independently selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S; or
R⁵ and R⁶ together with the carbon atoms to which they are attached form a 5- or 6-membered heterocycloalkyl having one or more heteroatoms selected from N, O and S; and
R⁷, R⁸, R⁹, R¹⁰, R¹¹, and R¹² are each independently selected from H, halogen, OH, CN, C₁-C₄ alkyl, C₁-C₄ haloalkyl, C₁-C₄ alkoxy, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S.

In embodiments,
L¹ is a bond, C₁-C₆ alkylene, C₁-C₆ haloalkylene, C₃-C₆ cycloalkylene, or a 5- or 6-membered heterocycloalkylene having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, C₉-C₁₀ bicyclic aryl, 5- or 6-membered heteroaryl having one or more heteroatoms selected from N, O and S, 8- to 10-membered bicyclic heteroaryl having one or more ring members selected from N, O, S and SO₂, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is selected from H, C₁-C₄ alkyl and C₃-C₈ cycloakyl;
R³ is selected from H, OH, CN, NR^{3a}R^{3b}, C₁-C₄ alkyl, C₃-C₈ cycloalkyl, 3- to 8-membered heterocycloalkyl having one or more heteroatoms selected from N, O and S, phenyl, and 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, CN, C₁-C₄ alkyl, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl; R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} and R^{4b} are each independently selected from H and C₁-C₄ alkyl;
R⁵ and R⁶ are each independently selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S; or
R⁵ and R⁶ together with the carbon atoms to which they are attached form a 5- or 6-membered heterocycloalkyl having one or more heteroatoms selected from N, O and S; and
R⁷, R⁸, R⁹, R¹⁰, R¹¹, and R¹² are each independently selected from H, halogen, OH, CN, C₁-C₄ alkyl, C₁-C₄ haloalkyl, C₁-C₄ alkoxy, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S.

In embodiments,
L¹ is a bond, C₁-C₆ alkylene, C₁-C₆ haloalkylene, C₃-C₆ cycloalkylene, or a 5- or 6-membered heterocycloalkylene having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, 8- to 10-membered bicyclic heteroaryl having one or more ring members selected from N, O, S and SO₂, and phenoxy, wherein said alkyl, cycloalkyl, heterocycloalkyl, bicyclic cycloalkyl, bicyclic heterocycloalkyl, phenyl, heteroaryl, bicyclic heteroaryl, and phenoxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, 5- or 6-membered heteroaryl having one or more heteroatoms selected from N, O and S, benzyl, halophenoxy, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, C₁-C₄ alkyl, and NR^{3c}R^{3d};
R^{3a} is selected from H, C₁-C₄ alkyl, and C₁-C₄ alkyl-NR^{3e}R^{3f},
R^{3b} is selected from H and C₁-C₄ alkyl;
R^{3c} is selected from H and C₁-C₄ alkyl;
R^{3d} is selected from C₁-C₄ alkyl;
R^{3e} and R^{3f} are each independently selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments,
L¹ is a bond, C₁-C₆ alkylene, C₁-C₆ haloalkylene, or C₃-C₆ cycloalkylene;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, 8- to 10-membered bicyclic heteroaryl having one or more ring members selected from N, O, S and SO₂, and phenoxy, wherein said alkyl, cycloalkyl, heterocycloalkyl, bicyclic cycloalkyl, bicyclic heterocycloalkyl, phenyl, heteroaryl, bicyclic heteroaryl, and phenoxy are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 5- or 6-membered heteroaryl having one or more heteroatoms selected from N, O and S, benzyl, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH and NR^{3c}R^{3d};
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments,
L¹ is a bond, C₁-C₆ alkylene, or C₁-C₆ haloalkylene;
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, and 5- or 6-membered heteroaryl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH and NR^{3c}R^{3d};
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from the following groups:

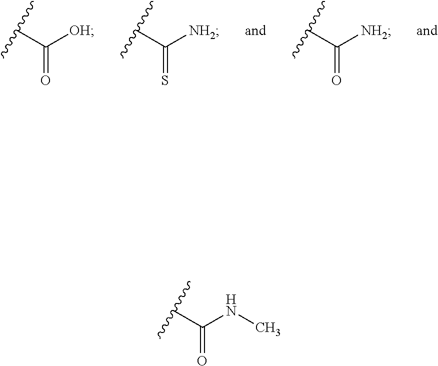

wherein [symbol] indicates the point of attachment to the rest of the compound;
R⁵ is selected from H and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and R⁷, R⁸, R⁹, R¹⁰, R¹¹, and R¹² are each H.

In embodiments,
L¹ is a bond, C₁-C₆ alkylene, or C₃-C₆ cycloalkylene;
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, and 8- to 10-membered bicyclic heteroaryl having one or more ring members selected from N, O, S and SO₂, wherein said cycloalkyl, heterocycloalkyl, bicyclic cycloalkyl, bicyclic heterocycloalkyl, phenyl, heteroaryl, and bicyclic heteroaryl are each optionally substituted by one or more substituents independently selected from halogen, OH, C₁-C₄ alkyl, C₁-C₄ haloalkyl, C₁-C₄ alkoxy, and C₁-C₄ haloalkoxy;
R² is H;
R³ is selected from H, CN, NR^{3a}R^{3b}, C₁-C₄ alkyl optionally substituted by OH, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₃-C₈ cycloalkyl-C₁-C₄ alkoxy, C₁-C₄ haloalkoxy-C₁-C₄ alkyl, C₁-C₄ alkyl-S—C₁-C₄ alkyl, and C₁-C₄ alkyl-SO₂-C₁-C₄ alkyl;
R^{3a} is H;
R^{3b} is selected from C₁-C₄ alkyl;
R⁴ is selected from the following groups:

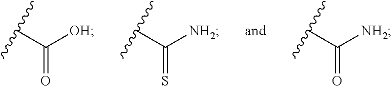

wherein [symbol] indicates the point of attachment to the rest of the compound;
R⁵ is H;
R⁶ is selected from H and C₁-C₄ alkyl; and
R⁷, R⁸, R⁹, R¹⁰, R¹¹, and R¹² are each H.

In embodiments,
L¹ is either a bond or C₁-C₆ alkylene;
R¹ is selected from C₃-C₈ cycloalkyl, C₈-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, OH, C₁-C₄ alkyl, C₁-C₄ haloalkyl, C₁-C₄ alkoxy, and C₁-C₄ haloalkoxy;
R² is H;
R³ is selected from NR^{3a}R^{3b}, C₁-C₄ alkyl optionally substituted by one or more substituents independently selected from OH and NR^{3c}R^{3d}, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₃-C₈ cycloalkoxy-C₁-C₄ alkyl, C₁-C₄ alkoxy-C₁-C₄ alkyl, C₁-C₄ haloalkoxy-C₁-C₄ alkyl, C₃-C₈ cycloalkyl-C₁-C₄ alkoxy, and alkyl-S—C₁-C₄ alkyl;
R^{3a} is H;
R^{3b} is selected from C₁-C₄ alkyl;
R⁴ is selected from the following groups:

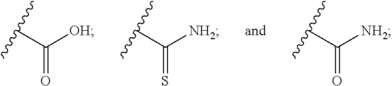

wherein [symbol] indicates the point of attachment to the rest of the compound R⁵, R⁷, R⁸, R⁹, R¹⁰, R¹¹, and R¹² are each H; and
R⁶ is selected from H and C₁-C₄ alkyl.

In embodiments, Q is O.

In embodiments, L is either a bond or methylene.

In embodiments, R¹ is selected from:
C₃-C₈ cycloalkyl optionally substituted by one or more substituents independently selected from halogen and C₁-C₄ haloalkoxy;
C₈-C₁₁ bicyclic cycloalkyl optionally substituted by one or more substituents independently selected from halogen, OH, C₁-C₄ alkyl and C₁-C₄ alkoxy;
3- to 8-membered heterocycloalkyl having one or more ring members selected from N, O, S and SO₂;
8- to 11-membered bicyclic heterocycloalkyl having one or more ring members selected from N, O, S and SO₂, optionally substituted by one or more substituents independently selected from halogen;
phenyl optionally substituted by one or more substituents independently selected from halogen and C₁-C₄ alkyl;
5- or 6-membered heteroaryl having one or more heteroatoms selected from N, O and S, optionally substituted by one or more substituents independently selected from C₁-C₄ alkyl and C₁-C₄ haloalkyl; and
8- to 10-membered bicyclic heteroaryl having one or more ring members selected from N, O, S and SO₂, optionally substituted substituted by one or more substituents independently selected from halogen and C₁-C₄ alkyl.

In embodiments, R² is H.

In embodiments, R³ is selected from:
NR^{3a}R^{3b}.
C₁-C₄ alkyl optionally substituted by OH;
C₃-C₈ cycloalkyl;
3- to 8-membered heterocycloalkyl having one or more heteroatoms selected from N, O and S;
5- or 6-membered heteroaryl having one or more heteroatoms selected from N, O and S;
C₁-C₄ alkoxy-C₁-C₄ alkyl;
C₃-C₈ cycloalkyl-C₁-C₄ alkoxy;
C₃-C₈ cycloalkoxy-C₁-C₄ alkyl;
C₁-C₄ haloalkoxy-C₁-C₄ alkyl; and
C₁-C₄ alkyl-S—C₁-C₄ alkyl;
   wherein

### R^{3a} is H; and

R^{3b} is selected from C₁-C₄ alkyl.

In embodiments, R³ is selected from C₁-C₄ alkoxy-C₁-C₄ alkyl.

In embodiments, R⁴ is selected from the following groups:

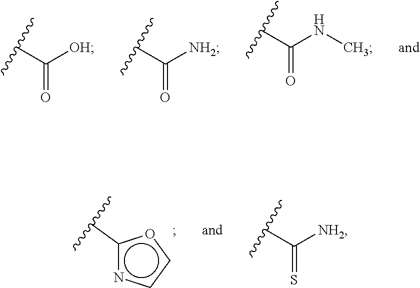

wherein [symbol] indicates the point of attachment to the rest of the compound.

In embodiments, R⁵ is H.

In embodiments, R⁶ is H or methyl.

In embodiments, R⁷, R⁸, R⁹, R¹⁰, R¹¹, and R¹² are each H.

In another aspect of this disclosure, there is provided a compound of formula (1) selected from Table 1. In another aspect there is provided a compound according to any one of compounds 1 to 242, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof;
for example a compound selected from compounds 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, and 242;
for example a compound selected from 1, 2, 4, 5, 6, 7, 8, 9, 11, 14, 15, 18, 19, 20, 21, 22, 25, 26, 27, 28, 29, 30, 31, 32, 33, 34, 35, 36, 37, 38, 39, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, and 242; for example a compound selected from 1, 2, 4, 5, 6, 9, 14, 18, 21, 22, 25, 26, 27, 29, 30, 32, 33, 34, 35, 36, 37, 39, 41, 42, 43, 44, 45, 46, 47, 48, 49, 50, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 91, 92, 93, 94, 95, 97, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, and 242;
for example a compound selected from 1, 2, 4, 6, 21, 26, 27, 33, 34, 37, 42, 43, 44, 46, 48, 53, 54, 55, 56, 57, 58, 59, 60, 61, 64, 65, 66, 67, 68, 69, 70, 71, 72, 73, 74, 75, 76, 78, 79, 80, 81, 82, 83, 85, 86, 87, 88, 91, 92, 93, 94, 104, 105, 106, 107, 108, 109, 110, 111, 112, 113, 114, 115, 116, 117, 118, 119, 120, 121, 122, 123, 126, 127, 128, 129, 130, 132, 133, 134, 135, 136, 137, 138, 139, 140, 141, 142, 143, 144, 145, 146, 147, 148, 149, 150, 151, 152, 153, 154, 155, 156, 157, 158, 159, 160, 162, 163, 164, 165, 166, 167, 168, 169, 170, 171, 172, 173, 174, 175, 176, 177, 178, 179, 180, 181, 182, 183, 184, 185, 189, 190, 191, 192, 193, 194, 195, 196, 198, 199, 200, 201, 202, 203, 204, 205, 206, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, and 242;
for example a compound selected from 27, 33, 34, 37, 46, 54, 56, 57, 58, 59, 60, 61, 64, 66, 68, 70, 72, 73, 74, 75, 79, 80, 82, 83, 85, 87, 88, 91, 92, 93, 105, 106, 107, 108, 109, 110, 111, 112, 113, 114, 115, 116, 117, 118, 119, 120, 123, 128, 129, 130, 133, 134, 135, 136, 137, 138, 139, 140, 141, 142, 144, 145, 147, 148, 150, 151, 152, 153, 157, 158, 159, 160, 163, 164, 166, 167, 168, 169, 170, 173, 174, 175, 176, 177, 178, 179, 182, 183, 184, 189, 191, 192, 193, 194, 195, 196, 200, 201, 202, 208, 209, 210, 211, 214, 216, 217, 219, 222, 223, 225, 226, 227, 228, 229, 230, 231, 232, 233, 234, 235, 236, 237, 238, 239, 240, and 242.

In another aspect of this disclosure, there is provided a compound of formula (1) as described above for use in medicine. In another aspect of this disclosure, there is provided a compound of formula (2aa) as described above for use in medicine. In another aspect of this disclosure, there is provided a compound of formula (2ab) as described above for use in medicine. In another aspect of this disclosure, there is provided a compound of formula (2ad) as described above for use in medicine.

In embodiments, the compound of formula (1), formula (2aa), (2ab), or (2ad) is for use in the treatment or prophylaxis of a cancer or proliferative disease or disorder.

In embodiments, the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML).

In another aspect of this disclosure, there is provided a pharmaceutical composition comprising a compound of formula (1), formula (2aa), formula (2ab) or formula (2ad) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier.

In another aspect of this disclosure, there is provided a pharmaceutical composition comprising a compound of formula (1), formula (2aa), formula (2ab), or formula (2ad) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier for use for use in medicine.

In embodiments, the pharmaceutical composition comprising a compound of formula (1), formula (2aa), formula (2ab), or formula (2ad) is for use in the treatment or prophylaxis of a cancer or proliferative disease or disorder.

In embodiments, the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML).

Within the scope of this disclosure it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. More particularly, it is specifically intended that any embodiment of any aspect may form an embodiment of any other aspect, and all such combinations are encompassed within the scope of the invention. The applicant reserves the right to change any originally filed claim or file any new claim accordingly, including the right to amend any originally filed claim to depend from and/or incorporate any feature of any other claim although not originally claimed in that manner.

### DETAILED DESCRIPTION

Described herein are compounds and compositions (e.g. organic molecules, research tools, pharmaceutical formulations and therapeutics); uses for the compounds and compositions of the disclosure (in vitro and in vivo); as well as corresponding methods, whether diagnostic, therapeutic or for research applications. The chemical synthesis and biological testing of the compounds of the disclosure are also described. Beneficially, the compounds, compositions, uses and methods have utility in research towards and/or the treatment of diseases or disorders in animals, such as humans. Diseases or disorders which may benefit from GPR68 modulation include, cancer and/or oncologic disease.

However, the compounds of the disclosure may also or alternatively be useful as lead molecules for the selection, screening and development of further derivatives that may have one or more improved beneficial drug property, as desired.

The disclosure also encompasses salts, solvates, tautomers, enantiomers and functional derivatives of the compounds described herein. These compounds may be useful in the treatment of various diseases or disorders; particularly those which may benefit from modulation of GPR68.

GPR68 modulators are useful in compositions and methods suitable for treating many disorders, and particularly cancer and/or proliferative diseases or disorders. In some embodiments, the disease is selected from the group consisting of adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In some embodiments, the disease is selected from the group consisting of colon cancer, medulloblastoma, head and neck cancer, myelodysplastic syndrome, pancreatic cancer, prostate cancer and melanoma. GPR68 modulators are also useful in compositions and methods suitable for treating fibrotic disease and inflammatory disease.

### Definitions

Unless defined otherwise, all technical and scientific terms used herein have the same meaning as commonly understood by one of ordinary skill in the art (e.g. in organic, physical or theoretical chemistry; biochemistry and molecular biology).

Unless otherwise indicated, the practice of the present invention employs conventional techniques in chemistry and chemical methods, biochemistry, molecular biology, pharmaceutical formulation, and delivery and treatment regimens for patients, which are within the capabilities of a person of ordinary skill in the art. Such techniques are also described in the literature cited herein, each of which is herein incorporated by reference in its entirety.

Prior to setting forth the detailed description of the invention, a number of definitions are provided that will assist in the understanding of the invention.

In accordance with the invention, the terms ‘molecule’ or ‘molecules’ are used interchangeably with the terms ‘compound’ or ‘compounds’, and sometimes the term ‘chemical structure’. The term ‘drug’ is typically used in the context of a pharmaceutical, pharmaceutical composition, medicament or the like, which has a known or predicted physiological or in vitro activity of medical significance; but such characteristics and qualities are not excluded in a molecule or compound of the invention. The term ‘drug’ is therefore used interchangeably with the alternatives terms and phrases ‘therapeutic (agent)’, ‘pharmaceutical (agent)’, and ‘active (agent)’. Therapeutics of the invention also encompass compositions and pharmaceutical formulations comprising the compounds of the invention.

It will be appreciated that certain compounds provided herein may contain one or more centres of asymmetry and may therefore be prepared and isolated in a mixture of isomers such as a racemic mixture, or in an enantiomerically pure form.

A compound or molecule as disclosed herein is meant to include all stereoisomers, geometric isomers, tautomers, and isotopically enriched variants of the structures depicted.

Compounds herein identified by name or structure as one particular tautomeric form are intended to include other tautomeric forms unless otherwise specified. The term ‘tautomer’, as used herein refers to compounds whose structures differ markedly in arrangement of atoms, but which exist in easy and rapid equilibrium, and it is to be understood that compounds provided herein may be depicted as different tautomers, and when compounds have tautomeric forms, all tautomeric forms are intended to be within the scope of the disclosure, and the naming of the compounds does not exclude any tautomer.

The term ‘substantially free of other stereoisomers’ as used herein means less than 10% of other stereoisomers, preferably less than 5% of other stereoisomers, more preferably less than 2% of other stereoisomers and most preferably less than 1% of other stereoisomers are present.

Prodrugs and solvates of the compounds of the invention are also encompassed within the scope of the invention. The term ‘prodrug’ means a compound (e.g. a drug precursor) that is transformed in vivo to yield a compound of the invention or a pharmaceutically acceptable salt, solvate or ester of the compound. The transformation may occur by various mechanisms (e.g. by metabolic or chemical processes), such as by hydrolysis of a hydrolysable bond, e.g. in blood (see Higuchi & Stella (1987), “Pro-drugs as Novel Delivery Systems”, vol. 14 of the A.C.S. Symposium Series; (1987), “Bioreversible Carriers in Drug Design”, Roche, ed., American Pharmaceutical Association and Pergamon Press). The compositions and medicaments of the invention therefore may comprise prodrugs of the compounds of the invention. In some aspects and embodiments, the compounds of the disclosure are themselves prodrugs which may be metabolised in vivo to give the therapeutically effective compound. For example, a sulfoxide prodrug may be metabolized in vivo to the therapeutically active sulfone (see Basarab G. S. et al., (2008), Bioorg Med Chem Lett, 18(16),4716-4722; Gibhard L. et al., (2008), Antimicrobial Agents and Chemotherapy, 62(12),00261-18).

The term ‘pharmaceutically acceptable salt’ as used herein refers to a salt that is not biologically or otherwise undesirable (e.g., not toxic or otherwise harmful). Thus, a ‘pharmaceutically acceptable’ compound is compatible chemically and/or toxicologically with the other ingredients comprising a formulation and/or the subject being treated therewith. A salt of a compound of the disclosure is formed between an acid and a basic group of the compound, or a base and an acidic group of the compound. For example, when the compounds of the disclosure contain at least one basic group (i.e., groups that can be protonated), the disclosure includes the compounds in the form of their acid addition salts with organic or inorganic acids such as, for example, but not limited to salts with hydrogen chloride, hydrogen bromide, phosphoric acid, sulfuric acid, nitric acid, benzenesulfonic acid, acetic acid, citric acid, glutamic acid, lactic acid, and methanesulfonic acid. When compounds of the disclosure contain one or more acidic groups (e.g., a carboxylic acid), the disclosure includes the pharmaceutically acceptable salts of the compounds formed with but not limited to alkali metal salts, alkaline earth metal salts or ammonium salts. Examples of such salts include, but are not limited to, sodium salts, potassium salts, calcium salts, magnesium salts or salts with ammonia or organic amines such as, for example, ethylamine, ethanolamine, triethanolamine or amino acids. Additional examples of such salts can be, found in Stahl, P. H. et al. Pharmaceutical Salts: Properties, Selection, and Use, 2^{nd} Revised Edition, Wiley, 2011.

In the context of the present disclosure, the terms ‘individual’, ‘subject’, or ‘patient’ are used interchangeably to indicate an animal that may be suffering from a medical (pathological) condition and may be responsive to a molecule, pharmaceutical drug, medical treatment or therapeutic treatment regimen of the disclosure. The animal is suitably a mammal, such as a human, cow, sheep, pig, dog, cat, bat, mouse or rat. In particular, the subject may be a human.

As used herein, the terms ‘treat’ ‘treating’ or ‘treatment’ include their generally accepted meanings in relation to therapeutic or palliative measures. Beneficial or desired clinical results in relation to the management and care of a patient or potential patient include, but are not limited to, alleviation or relief, in whole or in part, of symptoms associated with a disease, disorder or condition, diminishment of the extent of the disease, disorder or condition, a stabilized (i.e., not worsening) state of disease, delay or slowing of disease progression, amelioration or palliation of the disease state (e.g., one or more symptoms of the disease, disorder or condition), and remission (whether partial or total), whether detectable or undetectable. ‘Treatment’ can also mean prolonging survival as compared to expected survival if not receiving treatment.

The term ‘preventing’ as used herein means the prevention of the onset, recurrence or spread, in whole or in part, of the disease or condition as described herein, or a symptom thereof.

The term ‘therapeutically effective amount’ as used herein refers to that amount of compound of the disclosure that will elicit the biological or medical response of a tissue, system, animal, or human that is being sought by a researcher, veterinarian, medical doctor or other. As will be recognized by a person of ordinary skill in the art, a therapeutically effective amount of the compounds of the disclosure will vary and will depend on the disease treated, the severity of the disease, the route of administration, and the gender, age, and general health condition of the subject to whom the compound is being administered. The therapeutically effective amount may be administered as a single dose once a day, or as split doses administered multiple (e.g., two, three or four) times a day. The therapeutically effective amount may also be administered through continuous dosing, such as through infusion or with an implant.

Compounds provided herein may also contain unnatural proportions of atomic isotopes at one or more of the atoms that constitute such compounds. That is, an atom, in particular when mentioned in relation to a compound according to any of the formulas disclosed herein comprises all isotopes and isotopic mixtures of that atom, either naturally occurring or synthetically produced, either with natural abundance or in an isotopically enriched form. For example, when hydrogen is mentioned, it is understood to refer to ¹H, ²H, ³H or mixtures thereof; when carbon is mentioned, it is understood to refer to ¹¹C, ¹²C, ¹³C, ¹⁴C or mixtures thereof; when nitrogen is mentioned, it is understood to refer to ¹³N, ¹⁴N, ¹⁵N or mixtures thereof; when oxygen is mentioned, it is understood to refer to ¹⁴O, ¹⁵O, ¹⁶O, ¹⁷O, ¹⁸O or mixtures thereof; and when fluoro is mentioned, it is understood to refer to ¹⁸F, ¹⁹F or mixtures thereof; unless expressly noted otherwise. For example, in deuteroalkyl and deuteroalkoxy groups, where one or more hydrogen atoms are specifically replaced with deuterium (²H). As some of the aforementioned isotopes are radioactive, the compounds provided herein therefore also comprise compounds with one or more isotopes of one or more atoms, and mixtures thereof, including radioactive compounds, wherein one or more non-radioactive atoms has been replaced by one of its radioactive enriched isotopes.

Radiolabelled compounds are useful as therapeutic agents, e.g., cancer therapeutic agents, research reagents, e.g., assay reagents, and diagnostic agents, e.g., in vivo imaging agents.

All isotopic variations of the compounds provided herein, whether radioactive or not, are intended to be encompassed within the scope of the present disclosure.

In this regard, the term ‘deuterium’ as used herein refers to an isotope of hydrogen that has one proton and one neutron in its nucleus and that has twice the mass of ordinary hydrogen.

Deuterium herein is represented by the symbol ‘D’. The term ‘deuterated’ by itself or used to modify a compound or group as used herein refers to the presence of at least one deuterium atom attached to carbon. For example, the term ‘deuterated compound’ refers to a compound which contains one or more carbon-bound deuterium(s). In a deuterated compound of the present disclosure, when a particular position is designated as having deuterium, it is understood that the abundance of deuterium at that position is substantially greater than the natural abundance of deuterium, which is about 0.015%. The term ‘undeuterated’ or ‘non-deuterated’ as used herein refers to the ratio of deuterium atoms of which is not more than the natural isotopic deuterium content, which is about 0.015%; in other words, all hydrogens are present at their natural isotopic percentages. Unless otherwise stated, when a position is designated specifically as ‘H’ or ‘hydrogen’, the position is understood to have hydrogen at its natural abundance isotopic composition. However, it is explicitly intended that where H (or hydrogen) is mentioned as a substituent group in any of the aspects and embodiments of this disclosure, deuterium may also be a potential substituent at that position and can be considered implicitly recited as an optional substituent at each such position.

The term ‘isotopic enrichment factor’ as used herein refers to the ratio between the isotope abundance and the natural abundance of a specified isotope.

The term ‘isotopologue’ as used herein refers to a species in which the chemical structure differs from a specific compound of the disclosure only in the isotopic composition thereof.

The term ‘alkyl’ refers to a monovalent, optionally substituted, saturated aliphatic hydrocarbon radical. Any number of carbon atoms may be present, but typically the number of carbon atoms in the alkyl group may be from 1 to about 20, from 1 to about 12, from 1 to about 6 or from 1 to about 4. Usefully, the number of carbon atoms is indicated, for example, a C₁ C₄ alkyl refers to any alkyl group containing 1 to 4 carbon atoms in the chain. An alkyl group may be a straight chain (i.e. linear), branched chain, or cyclic. ‘Lower alkyl’ refers to an alkyl of 1 to 6 carbon atoms in the chain, and may have from 1 to 4 carbon atoms, or 1 to 2 carbon atoms. Thus, representative examples of lower alkyl radicals include methyl, ethyl, n-propyl, n-butyl, n-pentyl, n-hexyl, isopropyl, isobutyl, isopentyl, amyl (C₅H₁₁), sec-butyl, tert-butyl, sec-amyl, tert-pentyl, 2-ethylbutyl, 2,3-dimethylbutyl, and the like. ‘Higher alkyl’ refers to alkyls of 7 carbons and above, including n-heptyl, n-octyl, n-nonyl, n-decyl, n-dodecyl, n-tetradecyl, n-hexadecyl, n-octadecyl, n-eicosyl, and the like, along with branched variations thereof. A linear carbon chain of say 4 to 6 carbons would refer to the chain length not including any carbons residing on a branch, whereas in a branched chain it would refer to the total number. Optional substituents for alkyl and other groups are described below.

The term ‘alkoxy’ refers to a monovalent radical of the formula RO—, where R is any alkyl, alkenyl or alkynyl as defined herein. Alkoxy groups may be optionally substituted by any of the optional substituents described herein. ‘Lower alkoxy’ has the formula RO—, where the R group is a lower alkyl, alkenyl or alkynyl. Representative alkoxy radicals include methoxy, ethoxy, n-propoxy, n-butoxy, n-pentyloxy, n-hexyloxy, isopropoxy, isobutoxy, isopentyloxy, amyloxy, sec-butoxy, tert-butoxy, tert-pentyloxy, and the like. Preferred alkoxy groups are methoxy and ethoxy

The term ‘cycloalkyl’ as used herein refers to a cyclized alkyl ring having the indicated number of carbon atoms in a specified range. Thus, for example, ‘C₃-C₆ cycloalkyl’ encompasses each of cyclopropyl, cyclobutyl, cyclopentyl, and cyclohexyl.

The term ‘aryl’ as used herein refers to a substituted or unsubstituted aromatic carbocyclic radical containing from 5 to about 15 carbon atoms (‘C₆-C₁₅ aryl’); suitably 5 to 12 carbon atoms (‘C₅-C₁₂ aryl’) and more suitably 5 or 6 carbon atoms. An aryl group may have only one individual carbon ring, or may comprise one or more fused rings in which at least one ring is aromatic in nature. A ‘phenyl’ is a radical formed by removal of a hydrogen atom from a benzene ring, and may be substituted or unsubstituted. A ‘phenoxy’ group, therefore, is a radical of the formula RO—, wherein R is a phenyl radical. ‘Benzyl’ is a radical of the formula R—CH₂—, wherein R is phenyl, and ‘benzyloxy’ is a radical of the formula RO—, wherein R is benzyl. The point of attachment to the base molecule on such fused aryl ring systems may be a C atom of the aromatic portion or a C or a N atom of the non-aromatic portion of the ring system. Non-limiting examples of aryl radicals include, phenyl, naphthyl, anthracenyl, benzyl, biphenyl, furanyl, pyridinyl, indanyl, anthraquinolyl, tetrahydronaphthyl, a benzoic acid radical, a furan-2-carboxylic acid radical, and the like.

A ‘heteroaryl’ group is herein defined as a substituted or unsubstituted ‘aryl’ group in which one or more carbon atoms in the ring structure has been replaced with a heteroatom, such as nitrogen, oxygen or sulphur. Generally, the heteroaryl group contains one, two or three heteroatoms; particularly one or two heteroatoms. Particularly suitable heteroatoms are N and O; and a preferred heteroatom is N. Exemplary heteroaryl groups include: furan, benzofuran, isobenzofuran, pyrrole, indole, isoindole, thiophene, benzothiophene, benzo[c]thiophene, imidazole, benzimidazole, purine, pyrazole, indazole, oxazole, benzoxazole, isoxazole, benzisoxazole, thiazole, benzothiazole, pyridine, quinoline, isoquinoline, pyrazine, quinoxaline, acridine, pyrimidine, quinazoline, pyridazine and cinnoline.

Heteroaryl groups include but are not limited to thienyl (thiophenyl), benzo[b]thienyl, naphtho[2,3-b]thienyl, thianthrenyl, furyl (furanyl), isobenzofuranyl, chromenyl, xanthenyl, phenoxanthiinyl, pyrrolyl, including without limitation 2H-pyrrolyl, imidazolyl, pyrazolyl, pyridyl (pyridinyl), including without limitation 2-pyridyl, 3-pyridyl, and 4-pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalzinyl, naphthyridinyl, quinozalinyl, cinnolinyl, pteridinyl, carbazolyl, b-carbolinyl, phenanthridinyl, acrindinyl, pyrimidinyl, phenanthrolinyl, phenazinyl, thiazolyl, isothiazolyl, phenothiazinyl, oxazolyl, isoxazolyl, furazanyl, phenoxazinyl, 1,4-dihydroquinoxaline-2,3-dione, 7-aminoisocoumarin, pyrido[1,2-a]pyrimidin-4-one, pyrazolo[1,5-a]pyrimidinyl, including without limitation pyrazolo[1,5-a]pyrimidin-3-yl, 1,2-benzoisoxazol-3-yl, benzimidazolyl, 2-oxindolyl and 2-oxobenzimidazoly, oxadiazolyl, and thiadiazolyl. Where the heteroaryl group contains a nitrogen atom in a ring, such nitrogen atom may be in the form of an N-oxide, e.g., a pyridyl N-oxide, pyrazinyl N-oxide and pyrimidinyl N-oxide.

The terms ‘heterocycle’ or ‘heterocyclic’ group as used herein refer to a monovalent radical of from about 4- to about 15-ring atoms/members, and preferably 4-, 5- or 6-ring members. Generally, the heterocyclic group contains one, two or three heteroatoms, selected independently from nitrogen, oxygen and sulphur. Particularly suitable heteroatoms are N and O; and a preferred heteroatom is N. A heterocyclic group may have only one individual ring, or may comprise one or more fused rings in which at least one ring contains a heteroatom. It may be fully saturated or partially saturated, and may be substituted or unsubstituted as in the case or aryl and heteroaryl groups. Representative examples of unsaturated 5-membered heterocycles with only one heteroatom include 2- or 3-pyrrolyl, 2- or 3-furanyl, and 2- or 3-thiophenyl. Corresponding partially saturated or fully saturated radicals include 3-pyrrolin-2-yl, 2- or 3-pyrrolindinyl, 2- or 3-tetrahydrofuranyl, and 2- or 3-tetrahydrothiophenyl. Representative unsaturated 5-membered heterocyclic radicals having two heteroatoms include imidazolyl, oxazolyl, thiazolyl, pyrazolyl, and the like. The corresponding fully saturated and partially saturated radicals are also included.

Representative examples of unsaturated 6-membered heterocycles with only one heteroatom include 2-, 3-, or 4-pyridinyl, 2H-pyranyl, and 4H-pryanyl. Corresponding partially saturated or fully saturated radicals include 2-, 3-, or 4-piperidinyl, 2-, 3-, or 4-tetrahydropyranyl and the like. Representative unsaturated 6-membered heterocyclic radicals having two heteroatoms include 3- or 4-pyridazinyl, 2-, 4-, or 5-pyrimidinyl, 2-pyrazinyl, morpholino, and the like. The corresponding fully saturated and partially saturated radicals are also included, e.g. 2-piperazine. The heterocyclic radical is bonded through an available carbon atom or heteroatom in the heterocyclic ring directly to the entity or through a linker such as an alkylene such as methylene or ethylene.

The term ‘cycloaryl’ refers to a polycyclic group wherein an aryl group is fused to a 5- or 6-membered aliphatic ring. For example, C₆-C₁₂ cycloaryl means a C₆-C₁₂ aryl fused to a 5- or 6-membered aliphatic ring.

The term ‘heteroaryloxy’ or ‘heteroaryloxyl’ as used herein refers to an —O— heteroaryl group.

The disclosure encompasses fused ring systems, for example, ‘bicyclic’ or ‘tricyclic’ ring systems. In the context of the present disclosure, it is specifically intended that a fused ring system may include more than one fused aromatic ring, more than one fused non-aromatic/aliphatic ring, or one or more aromatic ring fused to one or more non-aromatic/aliphatic ring, such as a fusion of an aryl group with a cycloalkyl (or cycloalkenyl) group. Furthermore, it is intended that a fused ring system termed a bicyclic (or tricyclic)aryl is attached to the associated molecule via an aryl group, whereas a bicyclic (or tricyclic)cycloalkyl/cycloalkenyl is attached to the associated molecule via the cycloalkyl/cycloalkenyl group.

Similarly, in the context of fused ring systems, it is specifically intended that a bicyclic (or tricyclic) heteroaryl or heterocycloalkyl/heterocycloalkenyl need not contain heteroatoms in each of the fused ring systems. Rather, a bicyclic of tricyclic heteroaryl group may have one or more heteroatoms in any ring of the fused ring system, and not necessarily in the aryl ring that is the point of attachment to the associated molecule. Likewise, a bicyclic of tricyclic heterocycloalkyl or heterocycloalkenyl group may have one or more heteroatoms in any ring of the fused ring system, and not necessarily in the heterocycloalkyl or heterocycloalkenyl ring that is the point of attachment to the associated molecule.

The term ‘substituted’ means that one or more hydrogen atoms (attached to a carbon or heteroatom) is replaced with a selection from the indicated group of substituents, provided that the designated atom's normal valency under the existing circumstances is not exceeded. The group may be optionally substituted with particular substituents at positions that do not significantly interfere with the preparation of compounds falling within the scope of this invention and on the understanding that the substitution(s) does not significantly adversely affect the biological activity or structural stability of the compound. Combinations of substituents are permissible only if such combinations result in stable compounds. By ‘stable compound’ or ‘stable structure’, it is meant a compound that is sufficiently robust to survive isolation to a useful degree of purity from a reaction mixture and/or formulation into an efficacious therapeutic agent. The term ‘optionally substituted’ or ‘optional substituents’ as used herein means that the groups in question are either unsubstituted or substituted with one or more of the substituents specified. When the groups in question are substituted with more than one substituent, the substituents may be the same or different. By ‘optionally substituted’ it is meant that the group concerned is either unsubstituted, or at least one hydrogen atom is replaced with one of the specified substituent groups, radicals or moieties.

When used herein, the term ‘independently’ (e.g. in the phrase ‘independently selected from’), in reference to the substitution of a parent moiety with one or more substituents, means that the parent moiety may be substituted with any of the listed substituents, either individually or in combination, and any number of chemically possible substituents may be used. In any of the embodiments, where a group is substituted, it may contain up to 5, up to 4, up to 3, or 1 and 2 substituents. As a non-limiting example, useful substituents include: phenyl or pyridine, independently substituted with one or more lower alkyl, lower alkoxy or halo substituents, such as: chlorophenyl, dichlorophenyl, trichlorophenyl, tolyl, xylyl, 2-chloro-3-methylphenyl, 2,3-dichloro-4-methylphenyl, etc.

As used herein, the term ‘geminal’ refers to substituent atoms or groups attached to the same atom in a molecule. As used herein, the term ‘vicinal’ refers to substituent atoms or groups attached to adjacent atoms in a molecule. The stereochemical relationship between the substituent atoms or groups can be cis, trans, undefined, or unresolved.

Any radical/group/moiety described herein that may be substituted (or optionally substituted) may be substituted with one or more (e.g. one, two, three, four or five) substituents, which are independently selected from the designated group of substituents.

Thus, substituents may be selected from the group: halogen (or ‘halo’, e.g. F, Cl and Br), hydroxyl (—OH), amino(—NH₂), thiol (—SH), cyano (—CN), (lower) alkyl, (lower) alkoxy, (lower) alkenyl, (lower) alkynyl, aryl, heteroaryl, (lower) alkylthio, oxo, haloalkyl, hydroxyalkyl, nitro (—NO₂), phosphate, azido (—N₃), alkoxycarbonyl, carboxy, alkylcarboxy, alkylamino, dialkylamino, aminoalkyl, alkylaminoalkyl, dialkylaminoalkyl, thioalkyl, alkylsulfonyl, arylsulfinyl, alkylaminosulfonyl, arylaminosulfonyl, alkylsulfonylamino, arylsulfonylamino, carbamoyl, alkylcarbamoyl, dialkylcarbamoyl, arylcarbamoyl, alkylcarbonylamino, arylcarbonylamino, cycloalkyl, heterocycloalkyl, unless otherwise indicated. Alternatively, where the substituents are on an aryl or other cyclic ring system, two adjacent atoms may be substituted with a methylenedioxy or ethylenedioxy group. More suitably, the substituents are selected from: halogen, hydroxy, amino, thiol, cyano, (C₁-C₆)alkyl, (C₁-C₆)alkoxy, (C₁-C₆)alkenyl, (C₁-C₆)alkynyl, aryl, aryl(C₁-C₆)alkyl, aryl(C₁-C₆)alkoxy, heteroaryl, (C₁-C₆)alkylthio, oxo, halo(C₁-C₆)alkyl, hydroxy(C₁-C₆)alkyl, nitro, phosphate, azido, (C₁-C₆)alkoxycarbonyl, carboxy, (C₁-C₆)alkylcarboxy, (C₁-C₆)alkylamino, di(C₁-C₆)alkylamino, amino(C₁-C₆)alkyl, (C₁-C₆)alkylamino(C₁-C₆)alkyl, di(C₁-C₆)alkylamino(C₁-C₆)alkyl, thio(C₁-C₆)alkyl, (C₁-C₆)alkylsulfonyl, arylsulfinyl, (C₁-C₆)alkylaminosulfonyl, arylaminosulfonyl, (C₁-C₆)alkylsulfonylamino, arylsulfonylamino, carbamoyl, (C₁-C₆)alkylcarbamoyl, di(C₁-C₆)alkylcarbamoyl, arylcarbamoyl, (C₁-C₆)alkylcarbonylamino, arylcarbonylamino, (C₁-C₆)cycloalkyl, and heterocycloalkyl. Still more suitably, the substituents are selected from one or more of: fluoro, chloro, bromo, hydroxy, (C₁-C₆)alkyl, (C₁-C₆) haloalkyl, (C₁-C₆)alkoxy, (C₅-C₆)aryl, a 5- or 6-membered heteroaryl, (C₄-C₆)cycloalkyl, a 4- to 6-membered heterocycloalkyl, cyano, (C₁-C₆)alkylthio, amino, —NH(alkyl), —NH((C₁-C₆)cycloalkyl), —N((C₁-C₆)alkyl)₂, —OC(O)—(C₁-C₆)alkyl, —OC(O)—(C₅-C₆)aryl, —OC(O)—(C₁-C₆)cycloalkyl, carboxy and —C(O)O—(C₁-C₆)alkyl. Most suitably, the substituents are selected from one or more of: fluoro, chloro, bromo, hydroxy, amino, (C₁-C₆)alkyl and (C₁-C₆)alkoxy, wherein alkyl and alkoxy are optionally substituted by one or more chloro. Particularly preferred substituents are: chloro, methyl, ethyl, methoxy and ethoxy.

As used herein, the term ‘cyano’ refers to a —CN radical. As used herein, the term ‘hydroxyl’ refers to an —OH radical. As used herein, the term ‘amino’ refers to an —NH₂ group. As used herein, the term “oxo” refers to an “═O” group attached to a carbon atom.

The term ‘halo’ is used interchangeably with the term ‘halogen’ and refers to a monovalent halogen radical chosen from chloro, bromo, iodo, and fluoro. A ‘halogenated’ compound is one substituted with one or more halo substituent. Particular halo groups are F, Cl and Br; and most particularly are F or Cl. In some preferred embodiments the halo group is F.

The term ‘C₁-C₆ haloalkyl’ refers to a hydrocarbon chain substituted with at least one halogen atom independently chosen at each occurrence, for example fluorine, chlorine, bromine and iodine. The halogen atom may be present at any position on the hydrocarbon chain.

Similarly, a C₁-C₃ haloalkyl group is linear or branched hydrocarbon chain containing 1, 2, or 3 carbon atoms substituted with at least one halogen atom. For example, C₁-C₃ haloalkyl may refer to chloromethyl, fluoromethyl, trifluoromethyl, chloroethyl e.g. 1-chloroethyl and 2-chloroethyl, trichloroethyl e.g. 1,2,2-trichloroethyl, 2,2,2-trichloroethyl, fluoroethyl e.g. 1-fluoromethyl and 2-fluoroethyl, trifluoroethyl e.g. 1,2,2-trifluoroethyl and 2,2,2-trifluoroethyl, chloropropyl, trichloropropyl, fluoropropyl, trifluoropropyl.

‘Alkylene’ or ‘alkylenyl’ means a difunctional group obtained by removal of a hydrogen atom from an alkyl group as defined above. Non-limiting examples of alkylene include methylene, ethylene and propylene. ‘Lower alkylene’ means an alkylene having from 1 to 6 carbon atoms in the chain, and may be straight or branched. Alkylene groups are optionally substituted. Preferred alkylene groups are —(CR₂)ₙ— where n is from 1 to 6, and R is independent selected from H, F, Cl, NH₂ or wherein R₂ is ═O. More preferably, n is 1, 2 or 3 and R is H. Most preferable alkylene groups are —(CH₂)₂— and —CH₂—CO—.

The term ‘alkenyl’ refers to a monovalent, optionally substituted, unsaturated aliphatic hydrocarbon radical. Therefore, an alkenyl has at least one carbon-carbon double bond (C═C). The number of carbon atoms in the alkenyl group may be indicated, such as from 2 to about 20. For example, a C2-12 alkenyl (or C₂₋₁₂ alkenyl) refers to an alkenyl group containing 2 to 12 carbon atoms in the structure. Alkenyl groups may be straight (i.e. linear), branched chain, or cyclic. ‘Lower alkenyl’ refers to an alkenyl of 1 to 6 carbon atoms, and may have from 1 to 4 carbon atoms, or 1 to 2 carbon atoms. Representative examples of lower alkenyl radicals include ethenyl, 1-propenyl, 1-butenyl, 1-pentenyl, 1-hexenyl, isopropenyl, isobutenyl, and the like. Higher alkenyl refers to alkenyls of seven carbons and above, such as 1-heptenyl, 1-octenyl, 1-nonenyl, 1-decenyl, 1-dodecenyl, 1-tetradecenyl, 1-hexadecenyl, 1-octadecenyl, 1-eicosenyl, and the like, along with branched variations thereof. Optional substituents include are described elsewhere.

‘Alkenylene’ means a difunctional group obtained by removal of a hydrogen from an alkenyl group that is defined above. Non-limiting examples of alkenylene include —CH═CH—, —C(CH₃)═CH—, and —CH═CHCH₂—.

‘Alkynyl’ and ‘lower alkynyl’ is defined similarly to the term ‘alkenyl’, except that it includes at least one carbon-carbon triple bond.

Unless defined otherwise, ‘room temperature’ is intended to mean a temperature of from about 16 to 28° C., typically between about 18 and 25° C., and more typically between about 18 and 22° C. As used herein, the phrase ‘room temperature’ may be shortened to ‘rt’ or ‘RT’.

### Molecules and Compounds

Disclosed herein is a compound according to the invention having the structural formula (1):

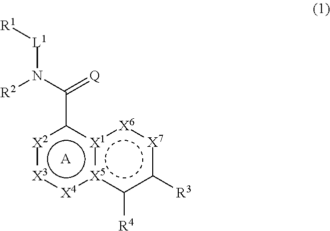

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein:
Q is O or S;
X¹ is selected from C and N;
X² is selected from CR⁵ and N;
X³ is selected from CR⁶ and N;
X⁴ is selected from CR⁷ and N;
X⁵ is selected from C and N;
wherein X¹, X², X³, X⁴ and X⁵ are selected so that the ring labelled A is aromatic;
X⁶ is selected from CR⁸R⁹, O and N;
X⁷ is selected from CR¹⁰, CR¹¹R¹², O and N;
q is 0 or 1;
wherein when q is 0, X⁷ is O or N; and when X⁷ is O or N and q is 1, X⁶ is CR⁸R⁹;
L¹ is a bond, alkylene, haloalkylene, cycloalkylene, or heterocycloalkyl;
R¹ is selected from alkyl, cycloalkyl, bicyclic cycloalkyl, heterocycloalkyl, bicyclic heterocycloalkyl, aryl, bicyclic aryl, heteroaryl, bicyclic heteroaryl, aryloxy, and heteroaryloxy, wherein said alkyl, cycloalkyl, bicyclic cycloalkyl, heterocycloalkyl, bicyclic heterocycloalkyl, aryl, bicyclic aryl, heteroaryl, bicyclic heteroaryl, aryloxy and heteroaryloxy are each optionally substituted by one or more substituents independently selected from halogen, OH, CN, NR^{1a}R^{1b}, alkyl, haloalkyl, alkoxy, haloalkoxy, alkyl-NR^{1a}R^{1b}, cycloalkyl, heterocycloakyl, aryl, heteroaryl, benzyl, aryloxy, haloaryloxy, heteroaryloxy, C₄ alkyl substituted by a heterocycloalkyl;
R^{1a} and R^{1b} are each independently selected from H and alkyl;
R² is selected from H, alkyl and cycloalkyl;
or R¹, R² and L¹ and the nitrogen atom to which R² and L¹ are attached form a heterocycloalkyl, bicyclic heterocycloalkyl, heteroaryl or bicyclic heteroaryl, wherein said heterocycloalkyl, bicyclic heterocycloalkyl, heteroaryl and bicyclic heteroaryl are each optionally substituted by one or more substituents independently selected from halogen, OH, CN, NR^{1a}R^{1b}, alkyl, haloalkyl, alkoxy, haloalkoxy and alkyl-NR^{1a}R^{1b};
R³ is selected from H, OH, CN, NR^{3a}R^{3b}, alkyl, cycloalkyl, heterocycloalkyl, aryl, heteroaryl, alkoxyalkyl, haloalkoxyalkyl, cycloalkoxyalkyl, cycloalkylalkoxy, alkyl-S-alkyl, and alkyl-SO₂-alkyl, wherein said alkyl, cycloalkyl, heterocycloalkyl, aryl and heteroaryl are each optionally substituted by one or more substituents independently selected from halogen, OH, CN, alkyl and NR^{3c}R^{3d};
R^{3a} is selected from H, alkyl, alkyl-OH, and alkyl-NR^{3e}R^{3f};
R^{3b}, R^{3c}, R^{3d}, R^{3e} and R^{3f} are each independently selected from H and alkyl;
R⁴ is selected from C(O)OH, C(O)NR^{4a}R^{4b}, C(S)NR^{4a}R^{4b} and heteroaryl;
R^{4a} and R^{4b} are each independently selected from H and alkyl;
R⁵ and R⁶ are each independently selected from H, halogen, OH, CN, alkyl, haloalkyl, alkoxy, alkoxyalkyl, cycloalkyl, heterocycloalkyl, aryl and heteroaryl;
or R⁵ and R⁶ together with the carbon atoms to which they are attached form a heterocycle; and
R⁷, R⁸, R⁹, R¹⁰, R¹¹ and R¹² are each independently selected from H, halogen, OH, CN, alkyl, haloalkyl, alkoxy, cycloalkyl, heterocycloalkyl, aryl and heteroaryl.

In embodiments of the compound of formula (1),
L¹ is a bond, C₁-C₆ alkylene, C₁-C₆ haloalkylene, C₃-C₆ cycloalkylene, or a 5- or 6-membered heterocycloalkyl having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, C₉-C₁₀ bicyclic aryl, 5- or 6-membered heteroaryl having one or more ring members selected from N, O, S and SO₂, 8- to 10-membered bicyclic heteroaryl having one or more ring members selected from N, O, S and SO₂, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is selected from H, C₁-C₄ alkyl and C₃-C₈ cycloakyl;
or R¹, R² and L¹ and the nitrogen atom to which R² and L¹ are attached form a 3- to 8-membered heterocycloalkyl having one or more heteroatoms selected from N, O and S, 8- to 11-membered bicyclic heterocycloalkyl having one or more heteroatoms selected from N, O and S, 5- or 6-membered heteroaryl having one or more heteroatoms selected from N, O and S, or 8- to 10-membered bicyclic heteroaryl having one or more heteroatoms selected from N, O and S, wherein said heterocycloalkyl, bicyclic heterocycloalkyl, heteroaryl and bicyclic heteroaryl are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy and C₁-C₄ alkyl-NR^{1a}R^{1b};
R³ is selected from H, OH, CN, NR^{3a}R^{3b}, C₁-C₄ alkyl, C₃-C₈ cycloalkyl, 3- to 8-membered heterocycloalkyl having one or more heteroatoms selected from N, O and S, phenyl, and 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, CN, C₁-C₄ alkyl, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} and R^{4b} are each independently selected from H and C₁-C₄ alkyl;
R⁵ and R⁶ are each independently selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S; or
R⁵ and R⁶ together with the carbon atoms to which they are attached form a 5- or 6-membered heterocycloalkyl having one or more heteroatoms selected from N, O and S; and
R⁷, R⁸, R⁹, R¹⁰, R¹¹, and R¹² are each independently selected from H, halogen, OH, CN, C₁-C₄ alkyl, C₁-C₄ haloalkyl, C₁-C₄ alkoxy, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S.

In embodiments of the compound of formula (1),
L¹ is a bond, C₁-C₆ alkylene, C₁-C₆ haloalkylene, C₃-C₆ cycloalkylene, or a 5- or 6-membered heterocycloalkyl having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, C₉-C₁₀ bicyclic aryl, 5- or 6-membered heteroaryl having one or more heteroatoms selected from N, O and S, 8- to 10-membered bicyclic heteroaryl having one or more ring members selected from N, O, S and SO₂, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is selected from H, C₁-C₄ alkyl and C₃-C₈ cycloakyl;
R³ is selected from H, OH, CN, NR^{3a}R^{3b}, C₁-C₄ alkyl, C₃-C₈ cycloalkyl, 3- to 8-membered heterocycloalkyl having one or more heteroatoms selected from N, O and S, phenyl, and 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, CN, C₁-C₄ alkyl, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} and R^{4b} are each independently selected from H and C₁-C₄ alkyl;
R⁵ and R⁶ are each independently selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S; or
R⁵ and R⁶ together with the carbon atoms to which they are attached form a 5- or 6-membered heterocycloalkyl having one or more heteroatoms selected from N, O and S; and
R⁷, R⁸, R⁹, R¹⁰, R¹¹, and R¹² are each independently selected from H, halogen, OH, CN, C₁-C₄ alkyl, C₁-C₄ haloalkyl, C₁-C₄ alkoxy, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S.

In embodiments of the compound of formula (1),
L¹ is a bond, C₁-C₆ alkylene, C₁-C₆ haloalkylene, C₃-C₆ cycloalkylene, or a 5- or 6-membered heterocycloalkyl having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, C₉-C₁₀ bicyclic aryl, 5- or 6-membered heteroaryl having one or more heteroatoms selected from N, O and S, 8- to 10-membered bicyclic heteroaryl having one or more ring members selected from N, O, S and SO₂, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, 5- or 6-membered heteroaryl having one or more heteroatoms selected from N, O and S, benzyl, halophenoxy, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is selected from H, C₁-C₄ alkyl and C₃-C₈ cycloakyl;
R³ is selected from H, OH, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, C₁-C₄ alkyl, and NR^{3c}R^{3d};
R^{3a} is selected from H, C₁-C₄ alkyl, and C₁-C₄ alkyl-NR^{3e}R^{3f};
R^{3b} is selected from H and C₁-C₄ alkyl;
R^{3c} is selected from H and C₁-C₄ alkyl;
R^{3d} is selected from C₁-C₄ alkyl;
R^{3e} and R^{3f} are each independently selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ and R⁶ are each independently selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S; or
R⁵ and R⁶ together with the carbon atoms to which they are attached form a 5- or 6-membered heterocycloalkyl having one or more heteroatoms selected from N, O and S; and
R⁷, R⁸, R⁹, R¹⁰, R¹¹, and R¹² are each independently selected from H, halogen, OH, CN, C₁-C₄ alkyl, C₁-C₄ haloalkyl, C₁-C₄ alkoxy, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S.

In embodiments of the compound of formula (1),
L¹ is a bond, C₁-C₆ alkylene, C₁-C₆ haloalkylene, C₃-C₆ cycloalkylene, or a 5- or 6-membered heterocycloalkyl having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, 8- to 10-membered bicyclic heteroaryl having one or more ring members selected from N, O, S and SO₂, and phenoxy, wherein said alkyl, cycloalkyl, heterocycloalkyl, bicyclic cycloalkyl, bicyclic heterocycloalkyl, phenyl, heteroaryl, bicyclic heteroaryl, and phenoxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, 5- or 6-membered heteroaryl having one or more heteroatoms selected from N, O and S, benzyl, halophenoxy, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, C₁-C₄ alkyl, and NR^{3c}R^{3d};
R^{3a} is selected from H, C₁-C₄ alkyl, and C₁-C₄ alkyl-NR^{3e}R^{3f};
R^{3b} is selected from H and C₁-C₄ alkyl;
R^{3c} is selected from H and C₁-C₄ alkyl;
R^{3d} is selected from C₁-C₄ alkyl;
R^{3e} and R^{3f} are each independently selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is HR⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments of the compound of formula (1),
L¹ is a bond, C₁-C₆ alkylene, C₁-C₆ haloalkylene, C₃-C₆ cycloalkylene, or a 5- or 6-membered heterocycloalkyl having one or more heteroatoms selected from N, O and S;
R¹ selected from:
   C₁-C₄ haloalkyl;
   C₃-C₈ cycloalkyl optionally substituted by one or more substituents independently selected from halogen, OH, CN, NR^{1a}R^{1b}, C₁-C₄ alkyl, C₁-C₄ alkoxy, and C₁-C₄ haloalkoxy;
   C₇-C₁₁ bicyclic cycloalkyl optionally substituted by one or more substituents independently selected from halogen, OH, C₁-C₄ alkyl, C₁-C₄ haloalkyl, C₁-C₄ alkoxy, benzyl, C₃-C₈ cycloalkyl, halophenoxy, 5- or 6-membered heteroaryl having one or more heteroatoms selected from N, O and S, and C₁-C₄ alkyl substituted by a 5- or 6-membered heterocycloalkyl having one or more heteroatoms selected from N, O and S;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, OH, C₁-C₄ alkyl, C₁-C₄ alkoxy, C₁-C₄ alkyl-NR^{1a}R^{1b}, and 5- or 6-membered heteroaryl having one or more heteroatoms selected from N, O and S;
   5- or 6-membered heteroaryl having one or more heteroatoms selected from N, O and S, optionally substituted by one or more substituents independently selected from C₁-C₄ alkyl and C₁-C₄ haloalkyl;
   8- to 10-membered bicyclic heteroaryl having one or more ring members selected from N, O, S and SO₂, optionally substituted by one or more substitutents independently selected from halogen, C₁-C₄ alkyl and C₁-C₄ alkoxy; and
   phenoxy optionally substituted by one or more C₁-C₄ alkyl;
R^{1a} and R^{1b} are each independently selected from H and C₁-C₄ alkyl;
R² is H;
R³ is selected from:
   H;
   CN;
   NR^{3a}R^{3b};
   C₁-C₄ alkyl optionally substituted by one of more substituents selected from OH and NR^{3c}R^{3d};
   C₃-C₈ cycloalkyl;
   3- to 8-membered heterocycloalkyl having one or more heteroatoms selected from N, O and S, optionally substituted by one or more C₁-C₄ alkyl;
   phenyl optionally substituted by one or more halogen;
   5- or 6-membered heteroaryl having one or more heteroatoms selected from N, O and S;
   C₁-C₄ alkoxy-C₁-C₄ alkyl;
   C₁-C₄ haloalkoxy-C₁-C₄ alkyl;
   C₃-C₈ cycloalkoxy-C₁-C₄ alkyl;
   C₃-C₈ cycloalkyl-C₁-C₄ alkoxy;
   C₁-C₄ alkyl-S—C₁-C₄ alkyl; and
   C₁-C₄ alkyl-SO₂-C₁-C₄ alkyl;
R^{3a} is selected from H, C₁-C₄ alkyl, and C₁-C₄ alkyl-NR^{3e}R^{3f};
R^{3b} is selected from H and C₁-C₄ alkyl;
R^{3c} is selected from H and C₁-C₄ alkyl;
R^{3d} is selected from C₁-C₄ alkyl;
R^{3e} and R^{3f} are each independently selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments of the compound of formula (1),
L¹ is a bond, C₁-C₆ alkylene, C₁-C₆ haloalkylene, C₃-C₆ cycloalkylene, or a 5- or 6-membered heterocycloalkyl having one or more heteroatoms selected from N, O and S;
R¹ selected from:
   C₁-C₄ haloalkyl;
   C₃-C₈ cycloalkyl optionally substituted by one or more substituents independently selected from halogen, OH, CN, NR^{1a}R^{1b}, C₁-C₄ alkyl, C₁-C₄ alkoxy, and C₁-C₄ haloalkoxy;
   C₇-C₁₁ bicyclic cycloalkyl optionally substituted by one or more substituents independently selected from halogen, OH, C₁-C₄ alkyl, C₁-C₄ haloalkyl, C₁-C₄ alkoxy, benzyl, C₃-C₈ cycloalkyl, halophenoxy, 5- or 6-membered heteroaryl having one or more heteroatoms selected from N, O and S, and C₁-C₄ alkyl substituted by a 5- or 6-membered heterocycloalkyl having one or more heteroatoms selected from N, O and S;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, OH, C₁-C₄ alkyl, C₁-C₄ alkoxy, C₁-C₄ alkyl-NR^{1a}R^{1b}, and 5- or 6-membered heteroaryl having one or more heteroatoms selected from N, O and S;
   5- or 6-membered heteroaryl having one or more heteroatoms selected from N, O and S, substituted by one or more substituents independently selected from C₁-C₄ alkyl and C₁-C₄ haloalkyl;
   8- to 10-membered bicyclic heteroaryl having one or more ring members selected from N, O, S and SO₂, optionally substituted by one or more substitutents independently selected from halogen, C₁-C₄ alkyl and C₁-C₄ alkoxy; and
   phenoxy substituted by one or more C₁-C₄ alkyl;
R^{1a} and R^{1b} are each independently selected from H and C₁-C₄ alkyl;
R² is H;
R³ is selected from:
   H;
   CN;
   NR^{3a}R^{3b};
   C₁-C₄ alkyl optionally substituted by one or more substituents selected from OH and NR^{3c}R^{3d};
   C₃-C₈ cycloalkyl;
   3- to 8-membered heterocycloalkyl having one or more heteroatoms selected from N, O and S, optionally substituted by one or more C₁-C₄ alkyl;
   phenyl optionally substituted by one or more halogen;
   5- or 6-membered heteroaryl having one or more heteroatoms selected from N, O and S;
   C₁-C₄ alkoxy-C₁-C₄ alkyl;
   C₁-C₄ haloalkoxy-C₁-C₄ alkyl;
   C₃-C₈ cycloalkoxy-C₁-C₄ alkyl;
   C₃-C₈ cycloalkyl-C₁-C₄ alkoxy;
   C₁-C₄ alkyl-S—C₁-C₄ alkyl; and
   C₁-C₄ alkyl-SO₂-C₁-C₄ alkyl;
R^{3a} is selected from H, C₁-C₄ alkyl, and C₁-C₄ alkyl-NR^{3e}R^{3f};
R^{3b} is selected from H and C₁-C₄ alkyl;
R^{3c} is selected from H and C₁-C₄ alkyl;
R^{3d} is selected from C₁-C₄ alkyl;
R^{3e} and R^{3f} are each independently selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments of the compound of formula (1),
L¹ is a bond, C₁-C₆ alkylene, C₁-C₆ haloalkylene, or C₃-C₆ cycloalkylene;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, 8- to 10-membered bicyclic heteroaryl having one or more ring members selected from N, O, S and SO₂, and phenoxy, wherein said alkyl, cycloalkyl, heterocycloalkyl, bicyclic cycloalkyl, bicyclic heterocycloalkyl, phenyl, heteroaryl, bicyclic heteroaryl, and phenoxy are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 5- or 6-membered heteroaryl having one or more heteroatoms selected from N, O and S, benzyl, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH and NR^{3c}R^{3d};
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments of the compound of formula (1),
L¹ is a bond, C₁-C₆ alkylene, C₁-C₆ haloalkylene, or C₃-C₆ cycloalkylene;
R¹ is selected from:
   C₁-C₄ haloalkyl;
   C₃-C₈ cycloalkyl optionally substituted by one or more substituents independently selected from halogen, OH, CN, C₁-C₄ alkyl, C₁-C₄ alkoxy, and C₁-C₄ haloalkoxy;
   C₇-C₁₁ bicyclic cycloalkyl optionally substituted by one or more substituents independently selected from halogen, OH, C₁-C₄ alkyl, C₁-C₄ haloalkyl, C₁-C₄ alkoxy, benzyl, C₃-C₈ cycloalkyl, and C₁-C₄ alkyl substituted by a 5- or 6-membered heterocycloalkyl having one or more heteroatoms selected from N, O and S;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, OH, C₁-C₄ alkyl, C₁-C₄ alkoxy, C₁-C₄ alkyl-NR^{1a}R^{1b}, and 5- or 6-membered heteroaryl having one or more heteroatoms selected from N, O and S;
   5- or 6-membered heteroaryl having one or more heteroatoms selected from N, O and S, optionally substituted by one or more substituents independently selected from C₁-C₄ alkyl and C₁-C₄ haloalkyl;
   8- to 10-membered bicyclic heteroaryl having one or more ring members selected from N, O, S and SO₂, optionally substituted by one or more substituents independently selected halogen, C₁-C₄ alkyl and C₁-C₄ alkoxy; and
   phenoxy optionally substituted by one or more C₁-C₄ alkyl;
R^{1a} and R^{1b} are each independently selected from H and C₁-C₄ alkyl;
R² is H;
R³ is selected from:
   H;
   CN;
   NR^{3a}R^{3b};
   C₁-C₄ alkyl optionally substituted by one or more substituents independently selected from OH and NR^{3c}R^{3d};
   C₃-C₈ cycloalkyl;
   3- to 8-membered heterocycloalkyl having one or more heteroatoms selected from N, O and S;
   phenyl substituted by one or more halogen;
   5- or 6-membered heteroaryl having one or more heteroatoms selected from N, O and S;
   C₁-C₄ alkoxy-C₁-C₄ alkyl;
   C₃-C₈ cycloalkyl-C₁-C₄ alkoxy;
   C₃-C₈ cycloalkoxy-C₁-C₄ alkyl;
   C₁-C₄ haloalkoxy-C₁-C₄ alkyl;
   C₁-C₄ alkyl-S—C₁-C₄ alkyl; and
   C₁-C₄ alkyl-SO₂-C₁-C₄ alkyl;
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments of the compound of formula (1),
L¹ is a bond, C₁-C₆ alkylene, C₁-C₆ haloalkylene, or C₃-C₆ cycloalkylene;
R¹ is selected from:
   C₁-C₄ haloalkyl;
   C₃-C₈ cycloalkyl optionally substituted by one or more substituents independently selected from halogen, OH, CN, C₁-C₄ alkyl, C₁-C₄ alkoxy, and C₁-C₄ haloalkoxy;
   C₇-C₁₁ bicyclic cycloalkyl optionally substituted by one or more substituents independently selected from halogen, OH, C₁-C₄ alkyl, C₁-C₄ haloalkyl, C₁-C₄ alkoxy, benzyl, C₃-C₈ cycloalkyl, and C₁-C₄ alkyl substituted by a 5- or 6-membered heterocycloalkyl having one or more heteroatoms selected from N, O and S;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, OH, C₁-C₄ alkyl, C₁-C₄ alkoxy, C₁-C₄ alkyl-NR^{1a}R^{1b}, and 5- or 6-membered heteroaryl having one or more heteroatoms selected from N, O and S;
   5- or 6-membered heteroaryl having one or more heteroatoms selected from N, O and S, substituted by one or more substituents independently selected from C₁-C₄ alkyl and C₁-C₄ haloalkyl;
   8- to 10-membered bicyclic heteroaryl having one or more ring members selected from N, O, S and SO₂, optionally substituted by one or more substituents independently selected from halogen, C₁-C₄ alkyl and C₁-C₄ alkoxy; and
   phenoxy substituted by one or more C₁-C₄ alkyl;
R^{1a} and R^{1b} are each independently selected from H and C₁-C₄ alkyl;
R² is H;
R³ is selected from:
   H;
   CN;
   NR^{3a}R^{3b};
   C₁-C₄ alkyl optionally substituted by one or more substituents independently selected from OH and NR^{3c}R^{3d};
   C₃-C₈ cycloalkyl;
   3- to 8-membered heterocycloalkyl having one or more heteroatoms selected from N, O and S;
   phenyl substituted by one or more halogen;
   5- or 6-membered heteroaryl having one or more heteroatoms selected from N, O and S;
   C₁-C₄ alkoxy-C₁-C₄ alkyl;
   C₃-C₈ cycloalkyl-C₁-C₄ alkoxy;
   C₃-C₈ cycloalkoxy-C₁-C₄ alkyl;
   C₁-C₄ haloalkoxy-C₁-C₄ alkyl;
   C₁-C₄ alkyl-S—C₁-C₄ alkyl; and
   C₁-C₄ alkyl-SO₂-C₁-C₄ alkyl;
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments of the compound of formula (1),
L¹ is a bond, C₁-C₆ alkylene, C₁-C₆ haloalkylene, or C₃-C₆ cycloalkylene;
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, and 5- or 6-membered heteroaryl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH and NR^{3c}R^{3d};
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from the following groups:

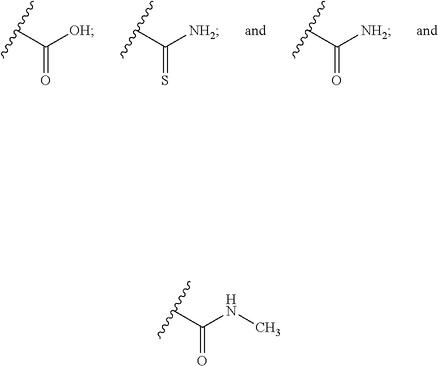

wherein [symbol] indicates the point of attachment to the rest of the compound;
R⁵ is selected from H and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments of the compound of formula (1),
L¹ is a bond, C₁-C₆ alkylene, C₁-C₆ haloalkylene, or C₃-C₆ cycloalkylene;
R¹ is selected from:
   C₃-C₈ cycloalkyl optionally substituted by one or more substituents independently selected from halogen, CN, C₁-C₄ alkyl, and C₁-C₄ haloalkoxy;
   C₇-C₁₁ bicyclic cycloalkyl optionally substituted by one or more substituents independently selected from halogen, OH, C₁-C₄ alkyl, C₁-C₄ haloalkyl, and C₁-C₄ alkoxy;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, OH, C₁-C₄ alkyl, C₁-C₄ alkoxy, C₁-C₄ alkyl-NR^{1a}R^{1b}, and 5- or 6-membered heteroaryl having one or more heteroatoms selected from N, O and S;
   5- or 6-membered heteroaryl having one or more heteroatoms selected from N, O and S, optionally substituted by one or more substituents independently selected from C₁-C₄ alkyl and C₁-C₄ haloalkyl; and
   8- to 10-membered bicyclic heteroaryl having one or more ring members selected from N, O, S and SO₂, optionally substituted substituted by one or more substituents independently selected from halogen and C₁-C₄ alkyl;
R^{1a} and R^{1b} are each independently selected from H and C₁-C₄ alkyl;
R² is H;
R³ is selected from:
   H;
   CN;
   NR^{3a}R^{3b}.
   C₁-C₄ alkyl optionally substituted by one or more substituents independently selected from OH and NR^{3c}R^{3d};
   C₃-C₈ cycloalkyl;
   3- to 8-membered heterocycloalkyl having one or more heteroatoms selected from N, O and S;
   phenyl substituted by one or more halogen;
   5- or 6-membered heteroaryl having one or more heteroatoms selected from N, O and S;
   C₁-C₄ alkoxy-C₁-C₄ alkyl;
   C₃-C₈ cycloalkyl-C₁-C₄ alkoxy;
   C₃-C₈ cycloalkoxy-C₁-C₄ alkyl;
   C₁-C₄ haloalkoxy-C₁-C₄ alkyl;
   C₁-C₄ alkyl-S—C₁-C₄ alkyl; and
   C₁-C₄ alkyl-SO₂-C₁-C₄ alkyl;
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from the following groups:

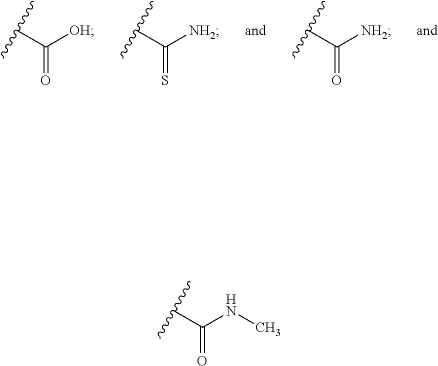

wherein [symbol] indicates the point of attachment to the rest of the compound;
R⁵ is selected from H and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments of the compound of formula (1),
L¹ is a bond, C₁-C₆ alkylene, C₁-C₆ haloalkylene, or C₃-C₆ cycloalkylene;
R¹ is selected from:
   C₃-C₈ cycloalkyl optionally substituted by one or more substituents independently selected from halogen, CN, C₁-C₄ alkyl, and C₁-C₄ haloalkoxy;
   C₇-C₁₁ bicyclic cycloalkyl optionally substituted by one or more substituents independently selected from halogen, OH, C₁-C₄ alkyl, C₁-C₄ haloalkyl, and C₁-C₄ alkoxy;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, OH, C₁-C₄ alkyl, C₁-C₄ alkoxy, C₁-C₄ alkyl-NR^{1a}R^{1b}, and 5- or 6-membered heteroaryl having one or more heteroatoms selected from N, O and S;
   5- or 6-membered heteroaryl having one or more heteroatoms selected from N, O and S, substituted by one or more substituents independently selected from C₁-C₄ alkyl and C₁-C₄ haloalkyl; and
   8- to 10-membered bicyclic heteroaryl having one or more ring members selected from N, O, S and SO₂, optionally substituted substituted by one or more substituents independently selected from halogen and C₁-C₄ alkyl;
R^{1a} and R^{1b} are each independently selected from H and C₁-C₄ alkyl;
R² is H;
R³ is selected from:
   H;
   CN;
   NR^{3a}R^{3b};
   C₁-C₄ alkyl optionally substituted by one or more substituents independently selected from OH and NR^{3c}R^{3d};
   C₃-C₈ cycloalkyl;
   3- to 8-membered heterocycloalkyl having one or more heteroatoms selected from N, O and S;
   phenyl substituted by one or more halogen;
   5- or 6-membered heteroaryl having one or more heteroatoms selected from N, O and S;
   C₁-C₄ alkoxy-C₁-C₄ alkyl;
   C₃-C₈ cycloalkyl-C₁-C₄ alkoxy;
   C₃-C₈ cycloalkoxy-C₁-C₄ alkyl;
   C₁-C₄ haloalkoxy-C₁-C₄ alkyl;
   C₁-C₄ alkyl-S—C₁-C₄ alkyl; and
   C₁-C₄ alkyl-SO₂-C₁-C₄ alkyl;
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from the following groups:

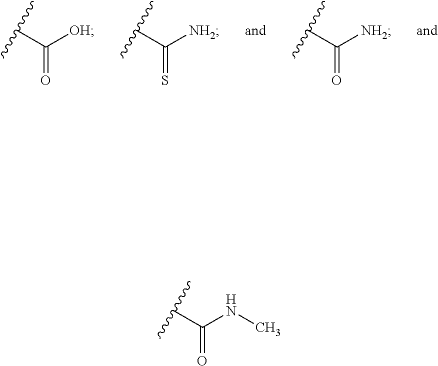

wherein [symbol] indicates the point of attachment to the rest of the compound;
R⁵ is selected from H, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments of the compound of formula (1),
L¹ is a bond, C₁-C₆ alkylene, or C₃-C₆ cycloalkylene;
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more heteroatoms selected from N, O and S, and 8- to 10-membered bicyclic heteroaryl having one or more ring members selected from N, O, S and SO₂, wherein said cycloalkyl, hetercycloalkyl, bicyclic cycloalkyl, bicyclic heterocycloalkyl, phenyl, heteroaryl, and bicyclic heteroaryl are each optionally substituted by one or more substituents independently selected from halogen, OH, C₁-C₄ alkyl, C₁-C₄ haloalkyl, C₁-C₄ alkoxy, and C₁-C₄ haloalkoxy;
R² is H;
R³ is selected from H, CN, NR^{3a}R^{3b}, C₁-C₄ alkyl optionally substituted by OH, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₃-C₈ cycloalkoxy-C₁-C₄ alkyl, C₃-C₈ cycloalkyl-C₁-C₄ alkoxy, C₁-C₄ haloalkoxy-C₁-C₄ alkyl, C₁-C₄ alkyl-S—C₁-C₄ alkyl, and C₁-C₄ alkyl-SO₂-C₁-C₄ alkyl;
R^{3a} is H;
R^{3b} is selected from C₁-C₄ alkyl;
R⁴ is selected from the following groups:

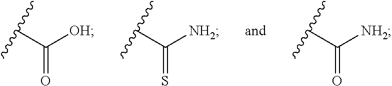

wherein [symbol] indicates the point of attachment to the rest of the compound;
R⁵ is H;
R⁶ is selected from H and C₁-C₄ alkyl; and
R⁷, R⁸, R⁹, R¹⁰, R¹¹, and R¹² are each H.

In embodiments of the compound of formula (1),
L¹ is either a bond, C₁-C₆ alkylene, or C₃-C₆ cycloalkylene;
R¹ is selected from:
   C₃-C₈ cycloalkyl optionally substituted by one or more substituents independently selected from halogen, C₁-C₄ alkyl, and C₁-C₄ haloalkoxy;
   C₇-C₁₁ bicyclic cycloalkyl optionally substituted by one or more substituents independently selected from halogen, OH, C₁-C₄ alkyl, and C₁-C₄ alkoxy;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, C₁-C₄ alkyl and C₁-C₄ alkoxy;
   5- or 6-membered heteroaryl having one or more ring members selected from N, O, S and SO₂, optionally substituted by one or more substituents independently selected from C₁-C₄ alkyl and C₁-C₄ haloalkyl; and
   8- to 10-membered bicyclic heteroaryl having one or more ring members selected from N, O, S and SO₂, optionally substituted substituted by one or more substituents independently selected from halogen and C₁-C₄ alkyl;
R² is H;
R³ is selected from:
   H;
   CN;
   NR^{3a}R^{3b};
   C₁-C₄ alkyl optionally substituted by OH;
   C₃-C₈ cycloalkyl;
   3- to 8-membered heterocycloalkyl having one or more heteroatoms selected from N, O and S;
   5- or 6-membered heteroaryl having one or more heteroatoms selected from N, O and S;
   C₁-C₄ alkoxy-C₁-C₄ alkyl;
   C₃-C₈ cycloalkyl-C₁-C₄ alkoxy;
   C₃-C₈ cycloalkoxy-C₁-C₄ alkyl
   C₁-C₄ haloalkoxy-C₁-C₄ alkyl;
   C₁-C₄ alkyl-S—C₁-C₄ alkyl; and
   C₁-C₄ alkyl-SO₂-C₁-C₄ alkyl;
R^{3a} is H;
R^{3b} is selected from C₁-C₄ alkyl.

R⁴ is selected from the following groups:

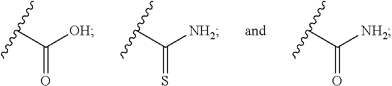

wherein [symbol] indicates the point of attachment to the rest of the compound;
R⁵ is H;
R⁶ is selected from H and C₁-C₄ alkyl; and
R⁷, R⁸, R⁹, R¹⁰, R¹¹, and R¹² are each H.

In embodiments of the compound of formula (1),
L¹ is either a bond, C₁-C₆ alkylene, or C₃-C₆ cycloalkylene;
R¹ is selected from:
   C₃-C₈ cycloalkyl optionally substituted by one or more substituents independently selected from halogen, C₁-C₄ alkyl, and C₁-C₄ haloalkoxy;
   C₇-C₁₁ bicyclic cycloalkyl optionally substituted by one or more substituents independently selected from halogen, OH, C₁-C₄ alkyl, and C₁-C₄ alkoxy;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, C₁-C₄ alkyl and C₁-C₄ alkoxy;
   5- or 6-membered heteroaryl having one or more ring members selected from N, O, S and SO₂, substituted by one or more substituents independently selected from C₁-C₄ alkyl and C₁-C₄ haloalkyl; and
   8- to 10-membered bicyclic heteroaryl having one or more ring members selected from N, O, S and SO₂, optionally substituted substituted by one or more substituents independently selected from halogen and C₁-C₄ alkyl;
R² is H;
R³ is selected from:
   H;
   CN;
   C₁-C₄ alkyl optionally substituted by OH;
   3- to 8-membered heterocycloalkyl having one or more heteroatoms selected from N, O and S;
   5- or 6-membered heteroaryl having one or more heteroatoms selected from N, O and S;
   C₁-C₄ alkoxy-C₁-C₄ alkyl;
   C₃-C₈ cycloalkyl-C₁-C₄ alkoxy;
   C₃-C₈ cycloalkoxy-C₁-C₄ alkyl;
   C₁-C₄ haloalkoxy-C₁-C₄ alkyl;
   C₁-C₄ alkyl-S—C₁-C₄ alkyl; and
   C₁-C₄ alkyl-SO₂-C₁-C₄ alkyl;
R^{3a} is H;
R^{3b} is selected from C₁-C₄ alkyl.

R⁴ is selected from the following groups:

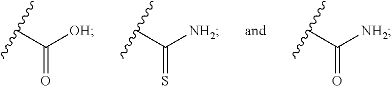

wherein [symbol] indicates the point of attachment to the rest of the compound;
R⁵ is H;
R⁶ is selected from H and C₁-C₄ alkyl; and
R⁷, R⁸, R⁹, R¹⁰, R¹¹, and R¹² are each H.

In embodiments of the compound of formula (1),
L¹ is either a bond or C₁-C₆ alkylene;
R¹ is selected from C₃-C₈ cycloalkyl, C₈-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, OH, C₁-C₄ alkyl, C₁-C₄ haloalkyl, C₁-C₄ alkoxy, and C₁-C₄ haloalkoxy;
R² is H;
R³ is selected from NR^{3a}R^{3b}, C₁-C₄ alkyl optionally substituted by one or more substituents independently selected from OH and NR^{3c}R^{3d}, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₃-C₈ cycloalkoxy-C₁-C₄ alkyl, C₁-C₄ alkoxy-C₁-C₄ alkyl, C₁-C₄ haloalkoxy-C₁-C₄ alkyl, C₃-C₈ cycloalkyl-C₁-C₄ alkoxy, and alkyl-S—C₁-C₄ alkyl;
R^{3a} is H;
R^{3b} is selected from C₁-C₄ alkyl;
R⁴ is selected from the following groups:

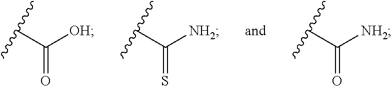

wherein [symbol] indicates the point of attachment to the rest of the compound;
R⁵, R⁷, R⁸, R⁹, R¹⁰, R¹¹, and R¹² are each H; and
R⁶ is selected from H and C₁-C₄ alkyl.

In embodiments of the compound of formula (1),
L¹ is either a bond or C₁-C₆ alkylene;
R¹ is selected from:
   C₃-C₈ cycloalkyl optionally substituted by one or more substituents independently selected from halogen and C₁-C₄ haloalkoxy;
   C₈-C₁₁ bicyclic cycloalkyl optionally substituted by one or more substituents independently selected from halogen, OH, C₁-C₄ alkyl and C₁-C₄ alkoxy;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen and C₁-C₄ alkyl;
   5- or 6-membered heteroaryl having one or more ring members selected from N, O, S and SO₂, optionally substituted by one or more substituents independently selected from C₁-C₄ alkyl and C₁-C₄ haloalkyl; and
   8- to 10-membered bicyclic heteroaryl having one or more ring members selected from N, O, S and SO₂, optionally substituted substituted by one or more substituents independently selected from halogen and C₁-C₄ alkyl;
R² is H;
R³ is selected from:
   NR^{3a}R^{3b};
   C₁-C₄ alkyl optionally substituted by OH;
   C₃-C₈ cycloalkyl;
   3- to 8-membered heterocycloalkyl having one or more heteroatoms selected from N, O and S;
   5- or 6-membered heteroaryl having one or more heteroatoms selected from N, O and S;
   C₁-C₄ alkoxy-C₁-C₄ alkyl;
   C₃-C₈ cycloalkyl-C₁-C₄ alkoxy;
   C₃-C₈ cycloalkoxy-C₁-C₄ alkyl;
   C₁-C₄ haloalkoxy-C₁-C₄ alkyl; and
   C₁-C₄ alkyl-S—C₁-C₄ alkyl;
R^{3a} is H;
R^{3b} is selected from C₁-C₄ alkyl;
R⁴ is selected from the following groups:

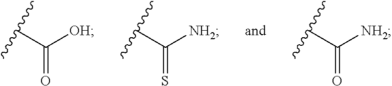

wherein [symbol] indicates the point of attachment to the rest of the compound H;
R⁵, R⁷, R⁸, R⁹, R¹⁰, R¹¹, and R¹² are each H; and
R⁶ is selected from H and C₁-C₄ alkyl.

In embodiments of the compound of formula (1),
L¹ is either a bond or C₁-C₆ alkylene;
R¹ is selected from:
   C₃-C₈ cycloalkyl substituted by one or more substituents independently selected from halogen and C₁-C₄ haloalkoxy;
   C₈-C₁₁ bicyclic cycloalkyl optionally substituted by one or more substituents independently selected from halogen, OH, C₁-C₄ alkyl and C₁-C₄ alkoxy;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl substituted by one or more substituents independently selected from halogen and C₁-C₄ alkyl;
   5- or 6-membered heteroaryl having one or more ring members selected from N, O, S and SO₂, substituted by one or more substituents independently selected from C₁-C₄ alkyl and C₁-C₄ haloalkyl; and
   8- to 10-membered bicyclic heteroaryl having one or more ring members selected from N, O, S and SO₂, substituted by one or more substituents independently selected from halogen and C₁-C₄ alkyl;
R² is H;
R³ is selected from:
   NR^{3a}R^{3b};
   C₁-C₄ alkyl optionally substituted by OH;
   C₃-C₈ cycloalkyl;
   3- to 8-membered heterocycloalkyl having one or more heteroatoms selected from N, O and S;
   5- or 6-membered heteroaryl having one or more heteroatoms selected from N, O and S;
   C₁-C₄ alkoxy-C₁-C₄ alkyl;
   C₃-C₈ cycloalkyl-C₁-C₄ alkoxy;
   C₃-C₈ cycloalkoxy-C₁-C₄ alkyl;
   C₁-C₄ haloalkoxy-C₁-C₄ alkyl; and
   C₁-C₄ alkyl-S—C₁-C₄ alkyl;
R^{3a} is H;
R^{3b} is selected from C₁-C₄ alkyl;
R⁴ is selected from the following groups:

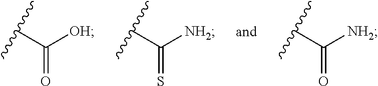

wherein [symbol] indicates the point of attachment to the rest of the compound;
R⁵, R⁷, R⁸, R⁹, R¹⁰, R¹¹, and R¹² are each H; and
R⁶ is selected from H and C₁-C₄ alkyl.

In embodiments of the compound of formula (1),

In embodiments of the compound of formula (1),
L¹ is a bond, C₁-C₆ alkylene, C₁-C₆ haloalkylene, C₃-C₆ cycloalkylene, or a 5- or 6-membered heterocycloalkylene having one or more heteroatoms selected from N, O and S.

In embodiments of the compound of formula (1),
L¹ is a bond, C₁-C₆ alkylene, C₁-C₆ haloalkylene, or C₃-C₆ cycloalkylene.

In embodiments of the compound of formula (1),
L¹ is a bond, methylene, ethylidene, 2,2,2-trifluoroethylidene, and cyclopropylidene.

In embodiments of the compound of formula (1),
L¹ is a bond, C₁-C₆ alkylene or a 5- or 6-membered heterocycloalkyl having one or more heteroatoms selected from N, O and S.

In embodiments of the compound of formula (1),
L¹ is either a bond or C₁-C₆ alkylene.

In embodiments of the compound of formula (1),
L¹ is either a bond or methylene.

In embodiments of the compound of formula (1),
L¹ is C₁-C₆ alkylene.

In embodiments of the compound of formula (1),
L¹ is methylene.

In embodiments of the compound of formula (1),
L¹ is a bond.

In embodiments of the compound of formula (1),
L¹ is a 5- or 6-membered heterocycloalkylene having one or more heteroatoms selected from N, O and S.

In embodiments of the compound of formula (1),
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, C₉-C₁₀ bicyclic aryl, 5- or 6-membered heteroaryl having one or more ring members selected from N, O, S and SO₂, 8- to 10-membered bicyclic heteroaryl having one or more ring members selected from N, O, S and SO₂, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more ring members selected from N, O, S and SO₂, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S; and
R^{1a} and R^{1b} are each independently selected from H and C₁-C₄ alkyl.

In embodiments of the compound of formula (1),
R¹ is selected from C₁-C₄ alkyl, C₃-C₈ cycloalkyl, C₈-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O,
S and SO₂, phenyl, C₉-C₁₀ bicyclic aryl, 5- or 6-membered heteroaryl having one or more ring members selected from N, O, S and SO₂, 8- to 10-membered bicyclic heteroaryl having one or more ring members selected from N, O, S and SO₂, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, C₉-C₁₀ bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S; and
R^{1a} and R^{1b} are each independently selected from H and C₁-C₄ alkyl.

In embodiments of the compound of formula (1),
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, 8- to 10-membered bicyclic heteroaryl having one or more ring members selected from N, O, S and SO₂, and phenoxy, wherein said alkyl, cycloalkyl, heterocycloalkyl, bicyclic cycloalkyl, bicyclic heterocycloalkyl, phenyl, heteroaryl, bicyclic heteroaryl, and phenoxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S; and
R^{1a} and R^{1b} are each independently selected from H and C₁-C₄ alkyl.

In embodiments of the compound of formula (1),
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, heterocycloalkyl, bicyclic cycloalkyl, bicyclic heterocycloalkyl, phenyl, heteroaryl, and bicyclic heteroaryl are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S; and
R^{1a} and R^{1b} are each independently selected from H and C₁-C₄ alkyl.

In embodiments of the compound of formula (1),
R¹ is selected from C₃-C₈ cycloalkyl, C₈-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S; and
R^{1a} and R^{1b} are each independently selected from H and C₁-C₄ alkyl.

In embodiments of the compound of formula (1),
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, 8- to 10-membered bicyclic heteroaryl having one or more ring members selected from N, O, S and SO₂, and phenoxy, wherein said alkyl, cycloalkyl, heterocycloalkyl, bicyclic cycloalkyl, bicyclic heterocycloalkyl, phenyl, heteroaryl, bicyclic heteroaryl, and phenoxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, 5- or 6-membered heteroaryl having one or more heteroatoms selected from N, O and S, benzyl, halophenoxy, and C₁-C₄ alkyl substituted by a 5- or 6-membered heterocycloalkyl having one or more heteroatoms selected from N, O and S; and
R^{1a} and R^{1b} are each independently selected from H and C₁-C₄ alkyl.

In embodiments of the compound of formula (1),
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, 5- or 6-membered heteroaryl having one or more heteroatoms selected from N, O and S, benzyl, halophenoxy, and C₁-C₄ alkyl substituted by a 5- or 6-membered heterocycloalkyl having one or more heteroatoms selected from N, O and S; and
R^{1a} and R^{1b} are each independently selected from H and C₁-C₄ alkyl.

In embodiments of the compound of formula (1),
R¹ is selected from C₃-C₈ cycloalkyl, C₈-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, 5- or 6-membered heteroaryl having one or more heteroatoms selected from N, O and S, benzyl, halophenoxy, and C₁-C₄ alkyl substituted by a 5- or 6-membered heterocycloalkyl having one or more heteroatoms selected from N, O and S; and
R^{1a} and R^{1b} are each independently selected from H and C₁-C₄ alkyl.

In embodiments of the compound of formula (1),
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, 8- to 10-membered bicyclic heteroaryl having one or more ring members selected from N, O, S and SO₂, and phenoxy, wherein said alkyl, cycloalkyl, heterocycloalkyl, bicyclic cycloalkyl, bicyclic heterocycloalkyl, phenyl, heteroaryl, bicyclic heteroaryl, and phenoxy are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 5- or 6-membered heteroaryl having one or more heteroatoms selected from N, O and S, benzyl, and C₁-C₄ alkyl substituted by a 5- or 6-membered heterocycloalkyl having one or more heteroatoms selected from N, O and S; and
R^{1a} and R^{1b} are each independently selected from H and C₁-C₄ alkyl.

In embodiments of the compound of formula (1),
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 5- or 6-membered heteroaryl having one or more heteroatoms selected from N, O and S, benzyl, and C₁-C₄ alkyl substituted by a 5- or 6-membered heterocycloalkyl having one or more heteroatoms selected from N, O and S; and
R^{1a} and R^{1b} are each independently selected from H and C₁-C₄ alkyl.

In embodiments of the compound of formula (1),
R¹ is selected from C₃-C₈ cycloalkyl, C₈-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 5- or 6-membered heteroaryl having one or more heteroatoms selected from N, O and S, benzyl, and C₁-C₄ alkyl substituted by a 5- or 6-membered heterocycloalkyl having one or more heteroatoms selected from N, O and S; and
R^{1a} and R^{1b} are each independently selected from H and C₁-C₄ alkyl.

In embodiments of the compound of formula (1),
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, and 5- or 6-membered heteroaryl having one or more heteroatoms selected from N, O and S; and
R^{1a} and R^{1b} are each independently selected from H and C₁-C₄ alkyl.

In embodiments of the compound of formula (1),
R¹ is selected from C₃-C₈ cycloalkyl, C₈-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, and 5- or 6-membered heteroaryl having one or more heteroatoms selected from N, O and S; and
R^{1a} and R^{1b} are each independently selected from H and C₁-C₄ alkyl.

In embodiments of the compound of formula (1),
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, heterocycloalkyl, bicyclic cycloalkyl, bicyclic heterocycloalkyl, phenyl, heteroaryl, and bicyclic heteroaryl are each optionally substituted by one or more substituents independently selected from halogen, OH,
C₁-C₄ alkyl, C₁-C₄ haloalkyl, C₁-C₄ alkoxy, and C₁-C₄ haloalkoxy.

In embodiments of the compound of formula (1),
R¹ is selected from C₃-C₈ cycloalkyl, C₈-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, heterocycloalkyl, bicyclic cycloalkyl, bicyclic heterocycloalkyl, phenyl, heteroaryl, and bicyclic heteroaryl are each optionally substituted by one or more substituents independently selected from halogen, OH, C₁-C₄ alkyl, C₁-C₄ haloalkyl, C₁-C₄ alkoxy, and C₁-C₄ haloalkoxy.

In embodiments of the compound of formula (1),
R¹ is selected from:
   C₁-C₄ haloalkyl;
   C₃-C₈ cycloalkyl optionally substituted by one or more substituents independently selected from halogen, OH, CN, NR^{1a}R^{1b}, C₁-C₄ alkyl, C₁-C₄ alkoxy, and C₁-C₄ haloalkoxy;
   C₇-C₁₁ bicyclic cycloalkyl optionally substituted by one or more substituents independently selected from halogen, OH, C₁-C₄ alkyl, C₁-C₄ haloalkyl, C₁-C₄ alkoxy, benzyl, C₃-C₈ cycloalkyl, halophenoxy, 5- or 6-membered heteroaryl having one or more heteroatoms selected from N, O and S, and C₁-C₄ alkyl substituted by a 5- or 6-membered heterocycloalkyl having one or more heteroatoms selected from N, O and S;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, OH, C₁-C₄ alkyl, C₁-C₄ alkoxy, C₁-C₄ alkyl-NR^{1a}R^{1b}, and 5- or 6-membered heteroaryl having one or more heteroatoms selected from N, O and S;
   5- or 6-membered heteroaryl having one or more ring members selected from N, O, S and SO₂, optionally substituted by one or more substituents independently selected from C₁-C₄ alkyl and C₁-C₄ haloalkyl;
   8- to 10-membered bicyclic heteroaryl having one or more ring members selected from N, O, S and SO₂, optionally substituted by one or more substitutents independently selected from halogen, C₁-C₄ alkyl and C₁-C₄ alkoxy; and
   phenoxy optionally substituted by one or more C₁-C₄ alkyl;
wherein R^{1a} and R^{1b} are each independently selected from H and C₁-C₄ alkyl.

In embodiments of the compound of formula (1),
R¹ is selected from:
   C₁-C₄ haloalkyl;
   C₃-C₈ cycloalkyl optionally substituted by one or more substituents independently selected from halogen, OH, CN, NR^{1a}R^{1b}, C₁-C₄ alkyl, C₁-C₄ alkoxy, and C₁-C₄ haloalkoxy;
   C₇-C₁₁ bicyclic cycloalkyl optionally substituted by one or more substituents independently selected from halogen, OH, C₁-C₄ alkyl, C₁-C₄ haloalkyl, C₁-C₄ alkoxy, benzyl, C₃-C₈ cycloalkyl, halophenoxy, 5- or 6-membered heteroaryl having one or more heteroatoms selected from N, O and S, and C₁-C₄ alkyl substituted by a 5- or 6-membered heterocycloalkyl having one or more heteroatoms selected from N, O and S;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, OH, C₁-C₄ alkyl, C₁-C₄ alkoxy, C₁-C₄ alkyl-NR^{1a}R^{1b}, and 5- or 6-membered heteroaryl having one or more ring members selected from N, O, S and SO₂ (e.g. having one or more ring members selected from N, O and S);
   5- or 6-membered heteroaryl having one or more ring members selected from N, O, S and SO₂, substituted by one or more substituents independently selected from C₁-C₄ alkyl and C₁-C₄ haloalkyl;
   8- to 10-membered bicyclic heteroaryl having one or more ring members selected from N, O, S and SO₂, optionally substituted by one or more substitutents independently selected from halogen, C₁-C₄ alkyl and C₁-C₄ alkoxy; and
   phenoxy substituted by one or more C₁-C₄ alkyl;
wherein R^{1a} and R^{1b} are each independently selected from H and C₁-C₄ alkyl.

In embodiments of the compound of formula (1),
R¹ is selected from:
   C₁-C₄ haloalkyl;
   C₃-C₈ cycloalkyl optionally substituted by one or more substituents independently selected from halogen, OH, CN, C₁-C₄ alkyl, C₁-C₄ alkoxy, and C₁-C₄ haloalkoxy;
   C₇-C₁₁ bicyclic cycloalkyl optionally substituted by one or more substituents independently selected from halogen, OH, C₁-C₄ alkyl, C₁-C₄ haloalkyl, C₁-C₄ alkoxy, benzyl, C₃-C₈ cycloalkyl, and C₁-C₄ alkyl substituted by a 5- or 6-membered heterocycloalkyl having one or more heteroatoms selected from N, O and S;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, OH, C₁-C₄ alkyl, C₁-C₄ alkoxy, C₁-C₄ alkyl-NR^{1a}R^{1b}, and 5- or 6-membered heteroaryl having one or more heteroatoms selected from N, O and S;
   5- or 6-membered heteroaryl having one or more ring members selected from N, O, S and SO₂, optionally substituted by one or more substituents independently selected from C₁-C₄ alkyl and C₁-C₄ haloalkyl;
   8- to 10-membered bicyclic heteroaryl having one or more ring members selected from N, O, S and SO₂, optionally substituted by one or more substituents independently selected from halogen, C₁-C₄ alkyl and C₁-C₄ alkoxy; and phenoxy optionally substituted by one or more C₁-C₄ alkyl;
wherein R^{1a} and R^{1b} are each independently selected from H and C₁-C₄ alkyl.

In embodiments of the compound of formula (1),
R¹ is selected from:
   C₁-C₄ haloalkyl;
   C₃-C₈ cycloalkyl optionally substituted by one or more substituents independently selected from halogen, OH, CN, C₁-C₄ alkyl, C₁-C₄ alkoxy, and C₁-C₄ haloalkoxy;
   C₇-C₁₁ bicyclic cycloalkyl optionally substituted by one or more substituents independently selected from halogen, OH, C₁-C₄ alkyl, C₁-C₄ haloalkyl, C₁-C₄ alkoxy, benzyl, C₃-C₈ cycloalkyl, and C₁-C₄ alkyl substituted by a 5- or 6-membered heterocycloalkyl having one or more heteroatoms selected from N, O and S;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, OH, C₁-C₄ alkyl, C₁-C₄ alkoxy, C₁-C₄ alkyl-NR^{1a}R^{1b}, and 5- or 6-membered heteroaryl having one or more heteroatoms selected from N, O and S;
   5- or 6-membered heteroaryl having one or more ring members selected from N, O, S and SO₂, substituted by one or more substituents independently selected from C₁-C₄ alkyl and C₁-C₄ haloalkyl;
   8- to 10-membered bicyclic heteroaryl having one or more ring members selected from N, O, S and SO₂, optionally substituted by one or more substituents independently selected from halogen, C₁-C₄ alkyl and C₁-C₄ alkoxy; and
   phenoxy substituted by one or more C₁-C₄ alkyl;
wherein R^{1a} and R^{1b} are each independently selected from H and C₁-C₄ alkyl.

In embodiments of the compound of formula (1),
R¹ is selected from:
   C₃-C₈ cycloalkyl optionally substituted by one or more substituents independently selected from halogen, CN, C₁-C₄ alkyl, and C₁-C₄ haloalkoxy;
   C₇-C₁₁ bicyclic cycloalkyl optionally substituted by one or more substituents independently selected from halogen, OH, C₁-C₄ alkyl, C₁-C₄ haloalkyl, and C₁-C₄ alkoxy;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, OH, C₁-C₄ alkyl, C₁-C₄ alkoxy, C₁-C₄ alkyl-NR^{1a}R^{1b}, and 5- or 6-membered heteroaryl having one or more heteroatoms selected from N, O and S;
   5- or 6-membered heteroaryl having one or more ring members selected from N, O, S and SO₂, optionally substituted by one or more substituents independently selected from C₁-C₄ alkyl and C₁-C₄ haloalkyl; and 8- to 10-membered bicyclic heteroaryl having one or more ring members selected from N, O, S and SO₂, optionally substituted substituted by one or more substituents independently selected from halogen and C₁-C₄ alkyl;
wherein R^{1a} and R^{1b} are each independently selected from H and C₁-C₄ alkyl.

In embodiments of the compound of formula (1),
R¹ is selected from:
   C₃-C₈ cycloalkyl optionally substituted by one or more substituents independently selected from halogen, CN, C₁-C₄ alkyl, and C₁-C₄ haloalkoxy;
   C₇-C₁₁ bicyclic cycloalkyl optionally substituted by one or more substituents independently selected from halogen, OH, C₁-C₄ alkyl, C₁-C₄ haloalkyl, and C₁-C₄ alkoxy;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, OH, C₁-C₄ alkyl, C₁-C₄ alkoxy, C₁-C₄ alkyl-NR^{1a}R^{1b}, and 5- or 6-membered heteroaryl having one or more heteroatoms selected from N, O and S;
   5- or 6-membered heteroaryl having one or more ring members selected from N, O, S and SO₂, substituted by one or more substituents independently selected from C₁-C₄ alkyl and C₁-C₄ haloalkyl; and
   8- to 10-membered bicyclic heteroaryl having one or more ring members selected from N, O, S and SO₂, optionally substituted substituted by one or more substituents independently selected from halogen and C₁-C₄ alkyl;
wherein R^{1a} and R^{1b} are each independently selected from H and C₁-C₄ alkyl.

In embodiments of the compound of formula (1),
R¹ is selected from:
   C₃-C₈ cycloalkyl optionally substituted by one or more substituents independently selected from halogen, C₁-C₄ alkyl, and C₁-C₄ haloalkoxy;
   C₇-C₁₁ bicyclic cycloalkyl optionally substituted by one or more substituents independently selected from halogen, OH, C₁-C₄ alkyl, and C₁-C₄ alkoxy;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, C₁-C₄ alkyl and C₁-C₄ alkoxy;
   5- or 6-membered heteroaryl having one or more ring members selected from N, O, S and SO₂, optionally substituted by one or more substituents independently selected from C₁-C₄ alkyl and C₁-C₄ haloalkyl; and
   8- to 10-membered bicyclic heteroaryl having one or more ring members selected from N, O, S and SO₂, optionally substituted substituted by one or more substituents independently selected from halogen and C₁-C₄ alkyl.

In embodiments of the compound of formula (1),
R¹ is selected from:
   C₃-C₈ cycloalkyl optionally substituted by one or more substituents independently selected from halogen, C₁-C₄ alkyl, and C₁-C₄ haloalkoxy;
   C₇-C₁₁ bicyclic cycloalkyl optionally substituted by one or more substituents independently selected from halogen, OH, C₁-C₄ alkyl, and C₁-C₄ alkoxy;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, C₁-C₄ alkyl and C₁-C₄ alkoxy;
   5- or 6-membered heteroaryl having one or more ring members selected from N, O, S and SO₂, substituted by one or more substituents independently selected from C₁-C₄ alkyl and C₁-C₄ haloalkyl; and
   8- to 10-membered bicyclic heteroaryl having one or more ring members selected from N, O, S and SO₂, optionally substituted substituted by one or more substituents independently selected from halogen and C₁-C₄ alkyl.

In embodiments of the compound of formula (1),
R¹ is selected from:
   C₃-C₈ cycloalkyl optionally substituted by one or more substituents independently selected from halogen and C₁-C₄ haloalkoxy;
   C₈-C₁₁ bicyclic cycloalkyl optionally substituted by one or more substituents independently selected from halogen, OH, C₁-C₄ alkyl and C₁-C₄ alkoxy;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen and C₁-C₄ alkyl;
   5- or 6-membered heteroaryl having one or more ring members selected from N, O, S and SO₂, optionally substituted by one or more substituents independently selected from C₁-C₄ alkyl and C₁-C₄ haloalkyl; and
   8- to 10-membered bicyclic heteroaryl having one or more ring members selected from N, O, S and SO₂, optionally substituted substituted by one or more substituents independently selected from halogen and C₁-C₄ alkyl.

In embodiments of the compound of formula (1),
R¹ is selected from:
   C₃-C₈ cycloalkyl substituted by one or more substituents independently selected from halogen and C₁-C₄ haloalkoxy;
   C₈-C₁₁ bicyclic cycloalkyl optionally substituted by one or more substituents independently selected from halogen, OH, C₁-C₄ alkyl and C₁-C₄ alkoxy;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl substituted by one or more substituents independently selected from halogen and C₁-C₄ alkyl;
   5- or 6-membered heteroaryl having one or more ring members selected from N, O, S and SO₂, substituted by one or more substituents independently selected from C₁-C₄ alkyl and C₁-C₄ haloalkyl; and
   8- to 10-membered bicyclic heteroaryl having one or more ring members selected from N, O, S and SO₂, substituted by one or more substituents independently selected from halogen and C₁-C₄ alkyl.

In embodiments of the compound of formula (1),
R¹ is selected from the following groups:

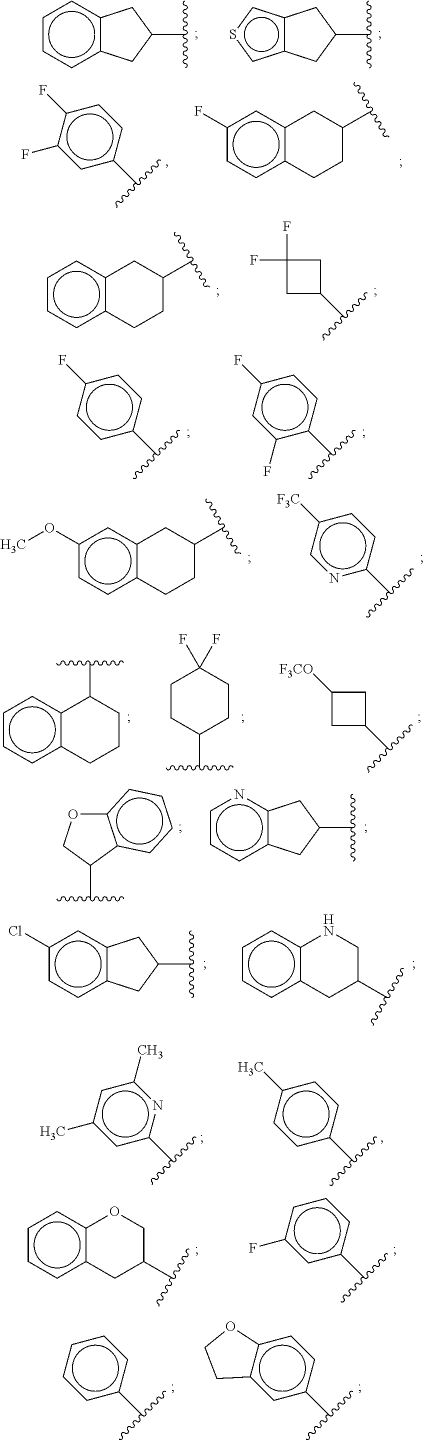

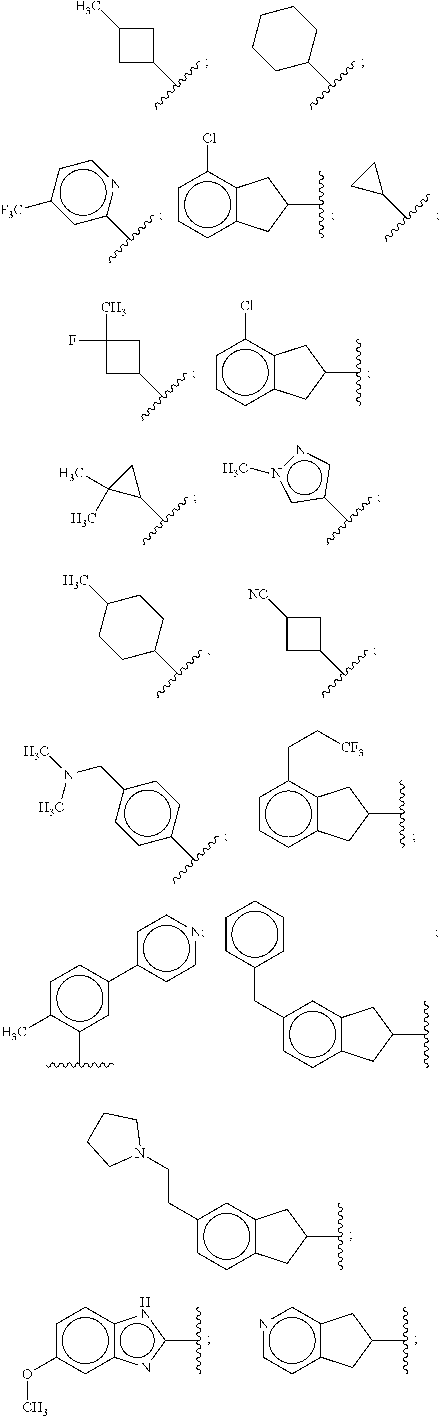

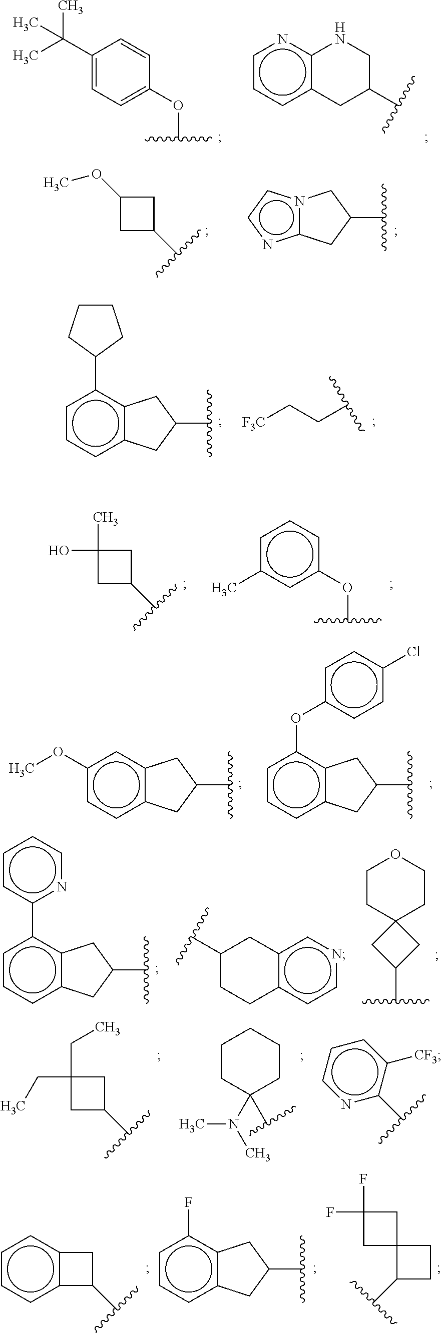

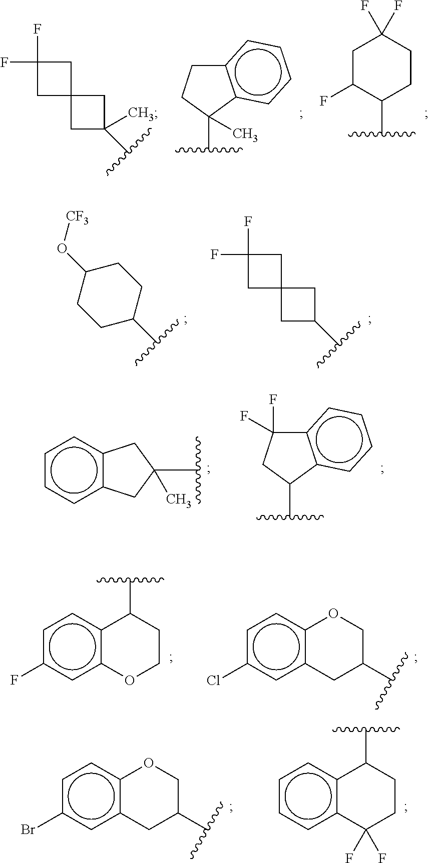

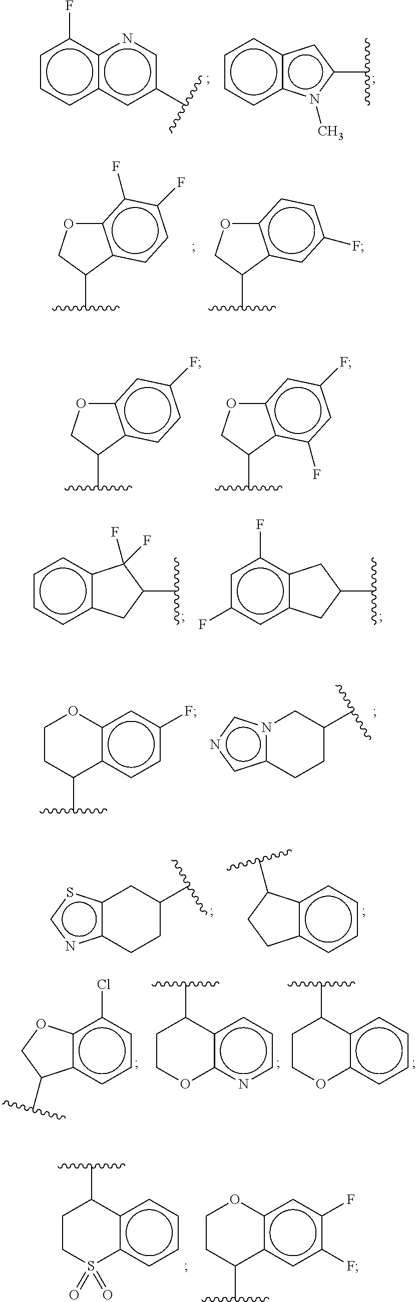

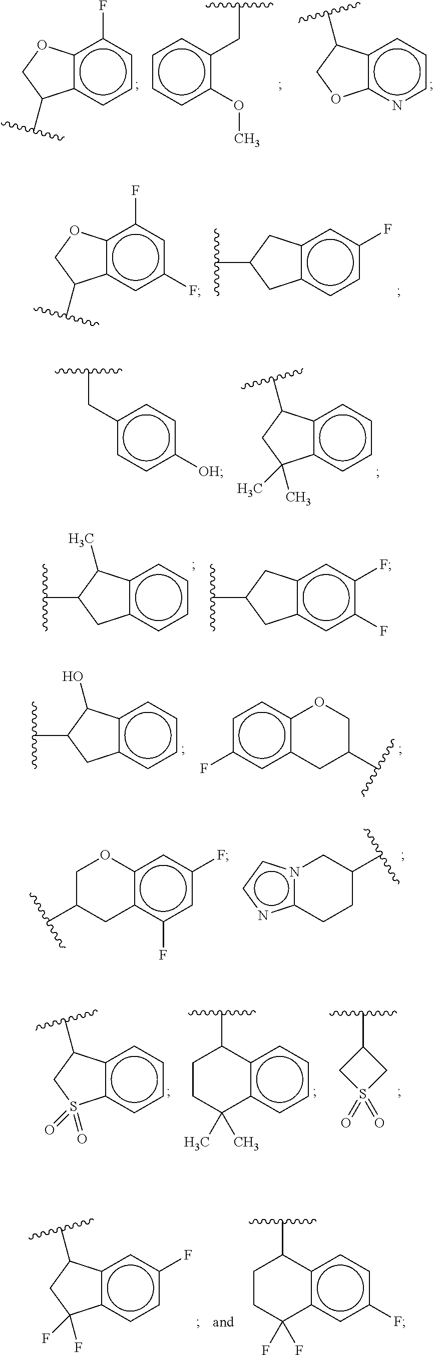

wherein [symbol] indicates the point of attachment to the rest of the compound.

In embodiments of the compound of formula (1),
R¹ is selected from the following groups:

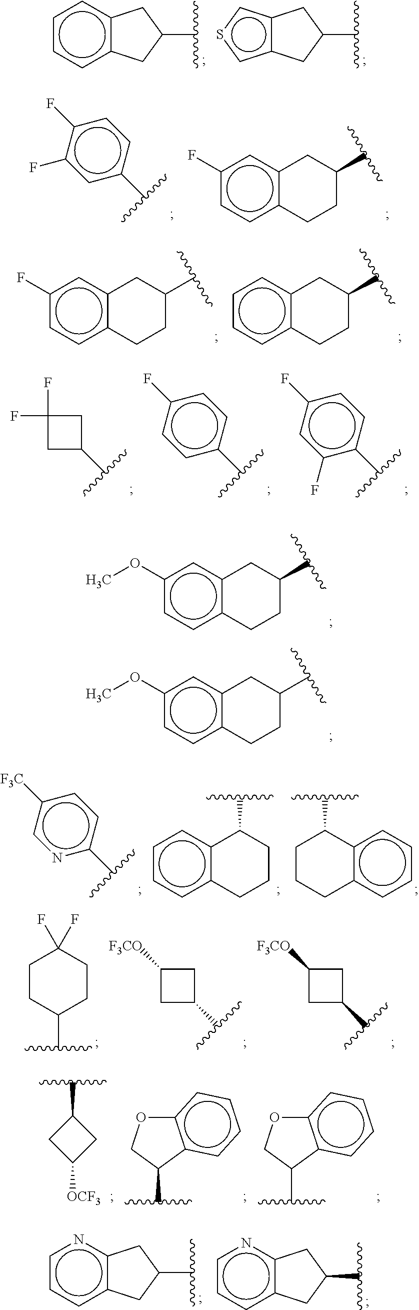

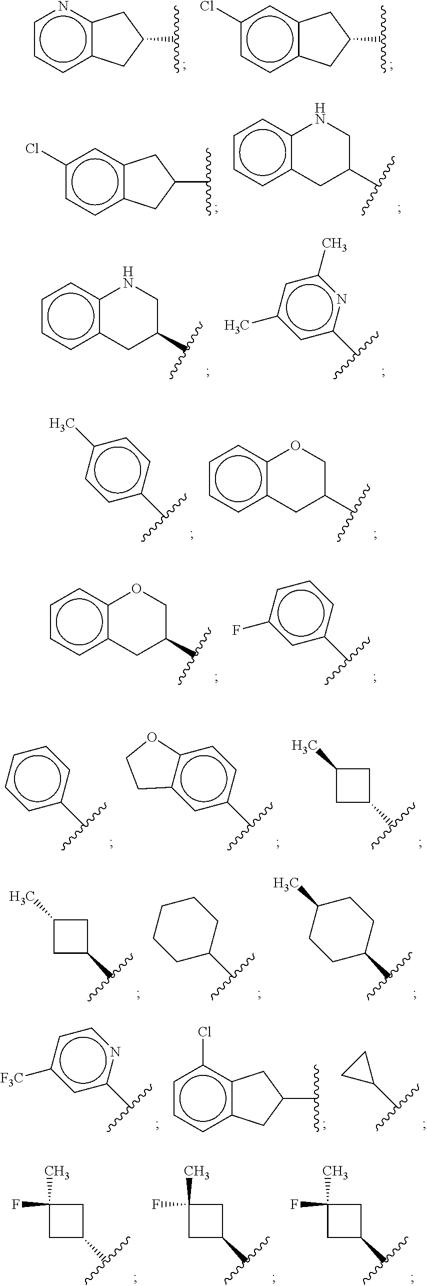

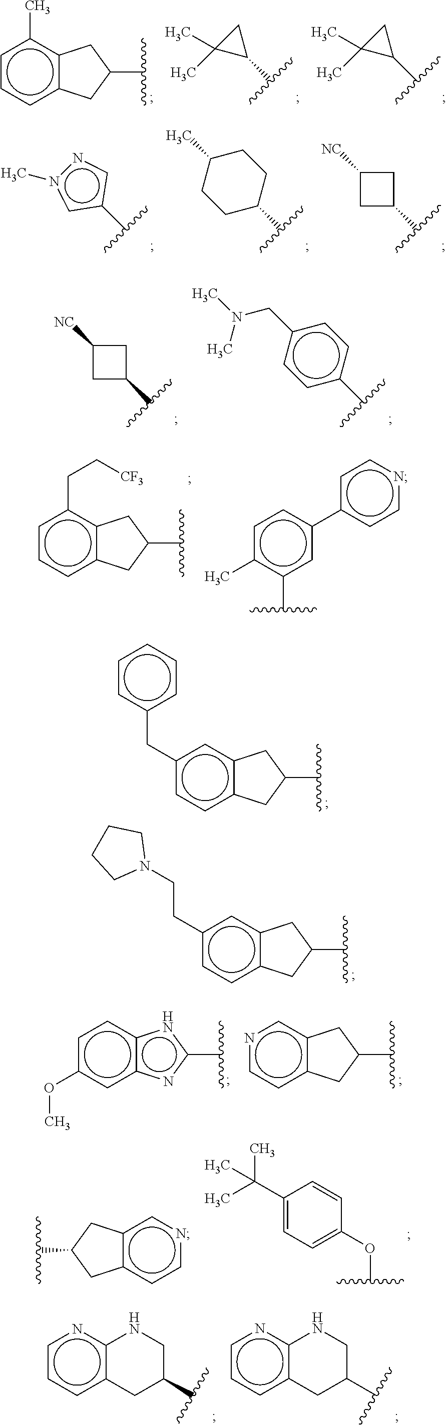

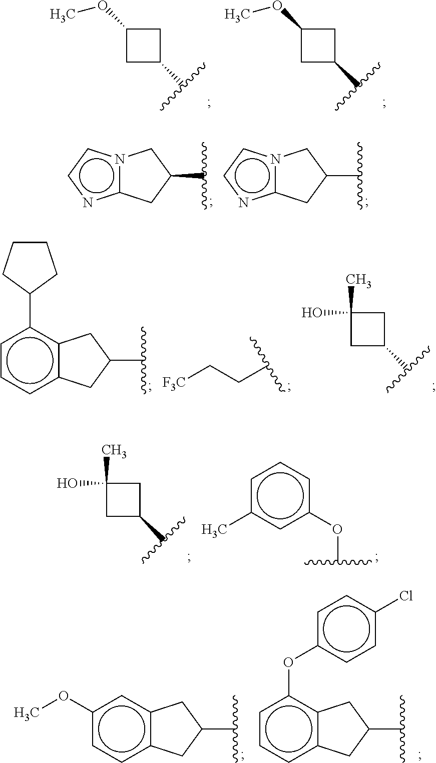

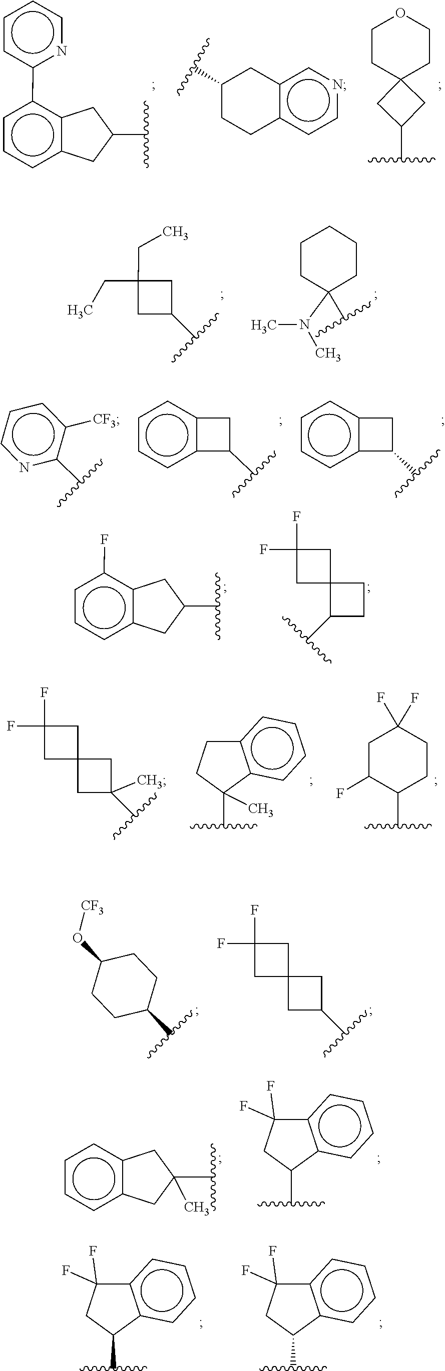

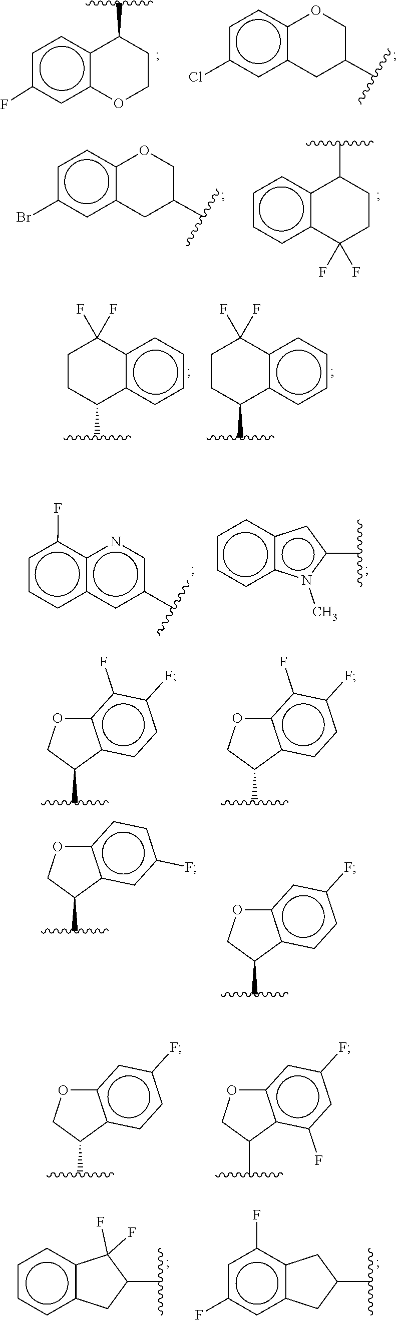

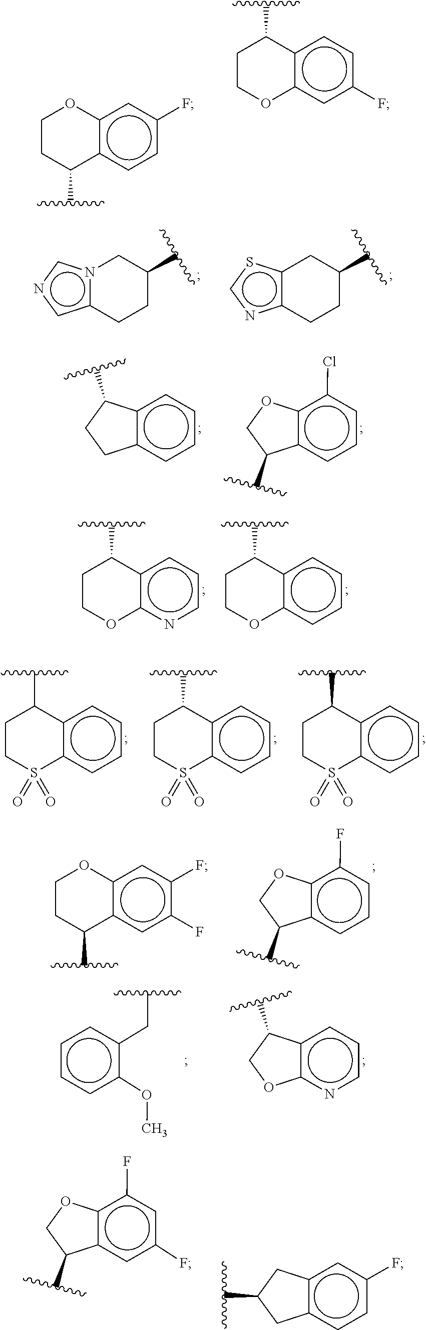

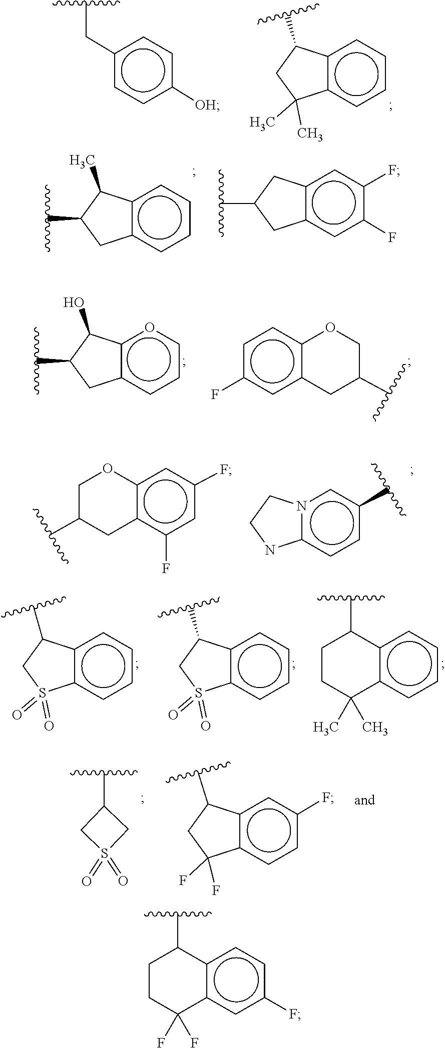

wherein [symbol] indicates the point of attachment to the rest of the compound.

In embodiments of the compound of formula (1),
R¹ is selected from the following groups:

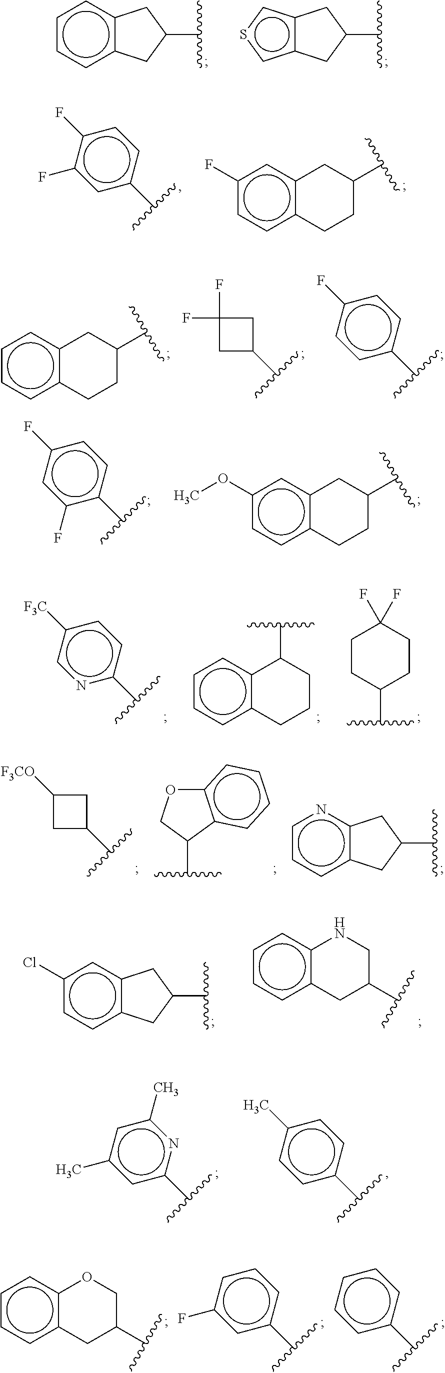

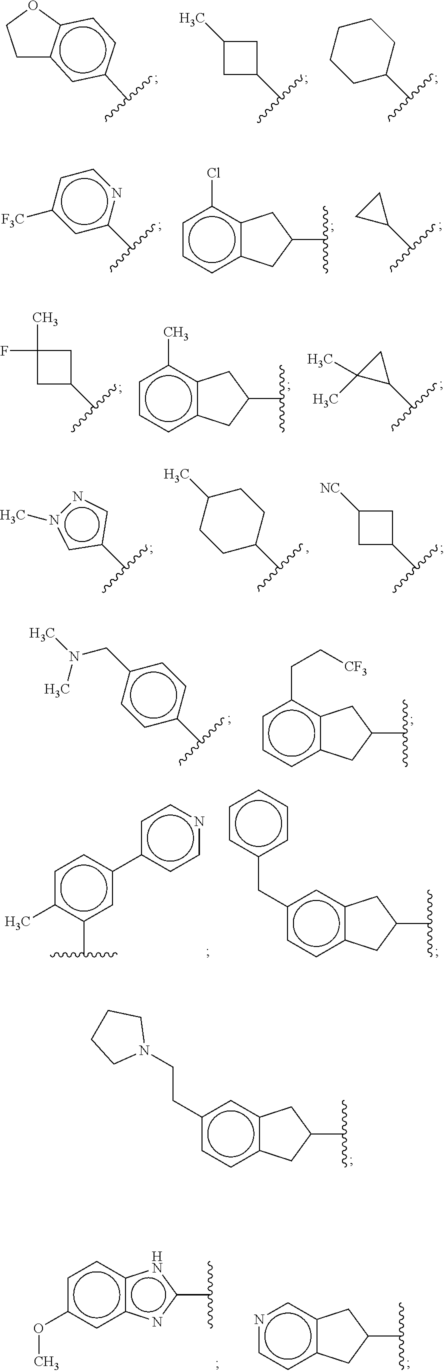

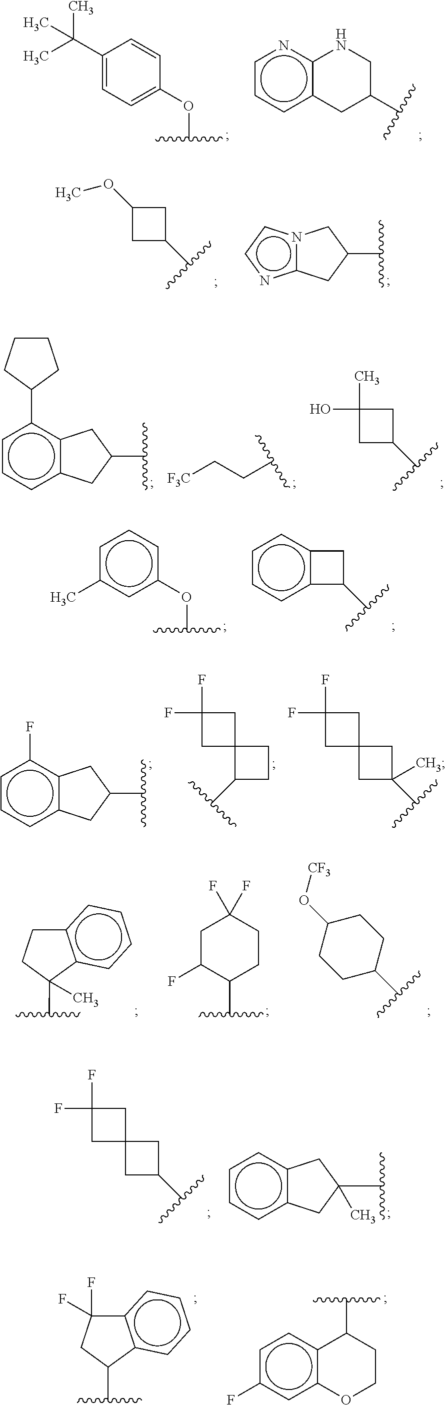

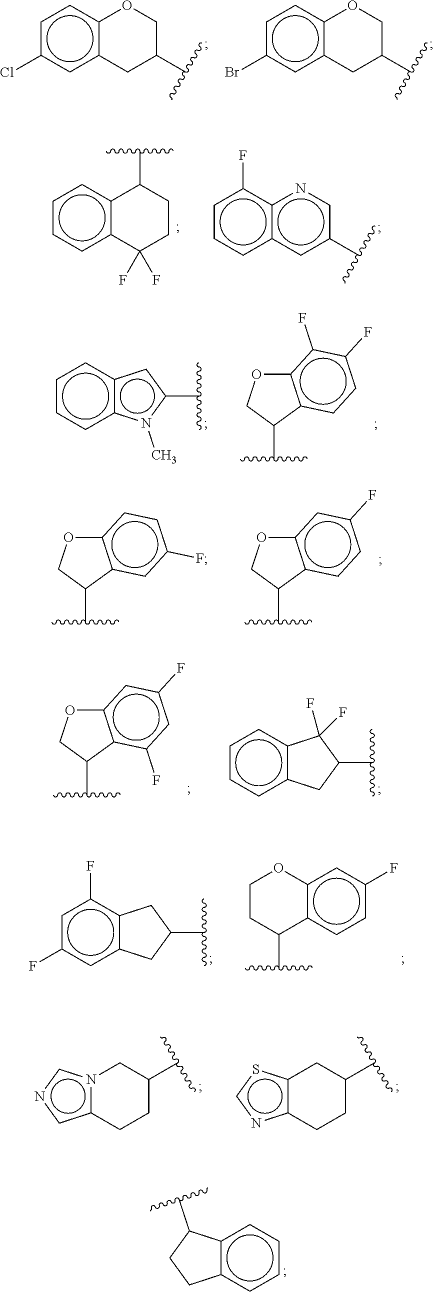

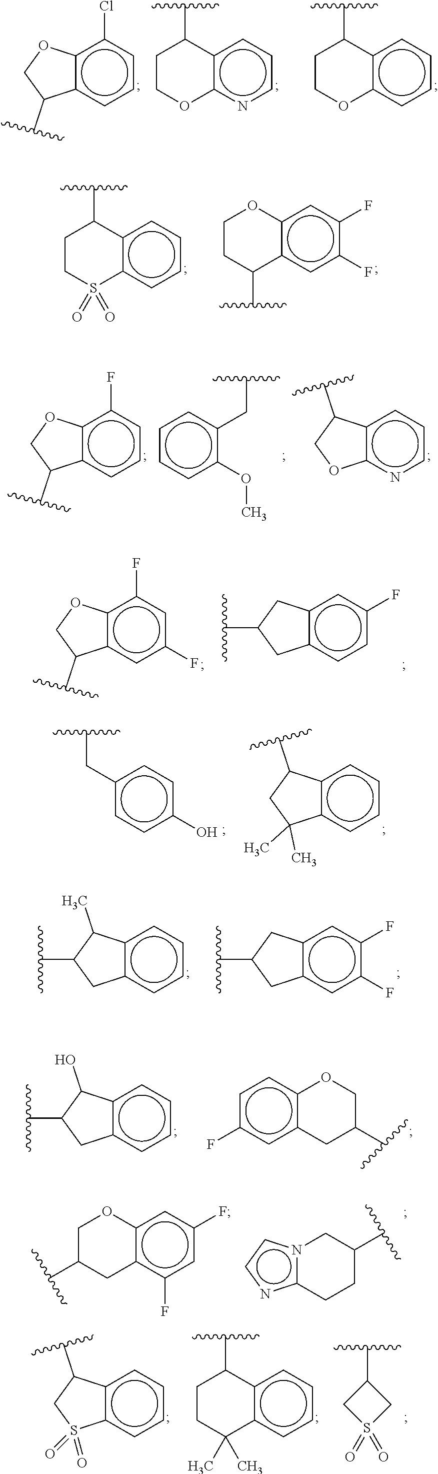

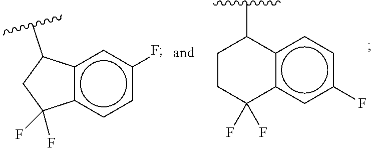

wherein [symbol] indicates the point of attachment to the rest of the compound.

In embodiments of the compound of formula (1),
R¹ is selected from the following groups:

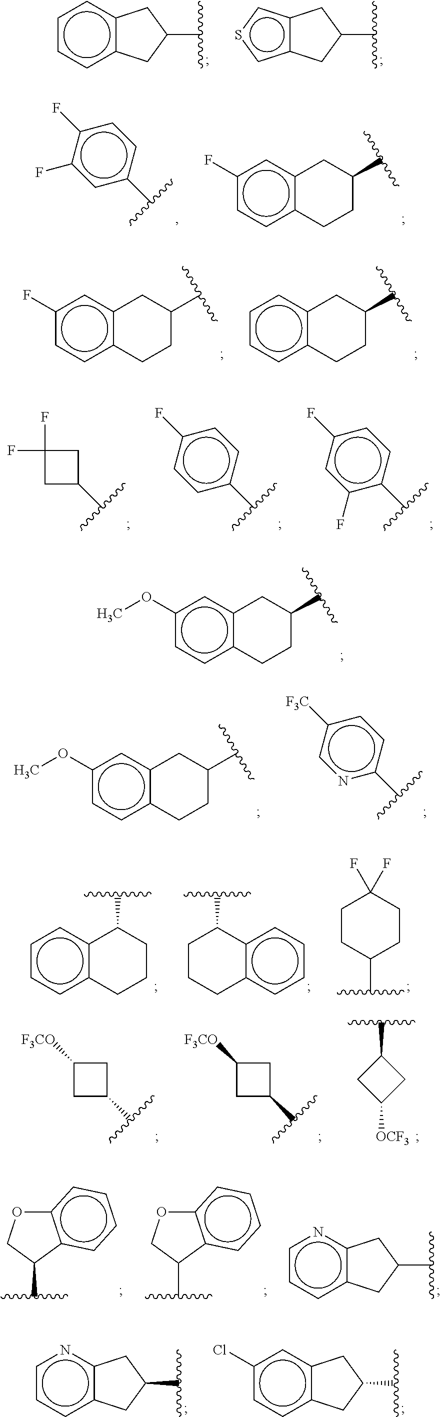

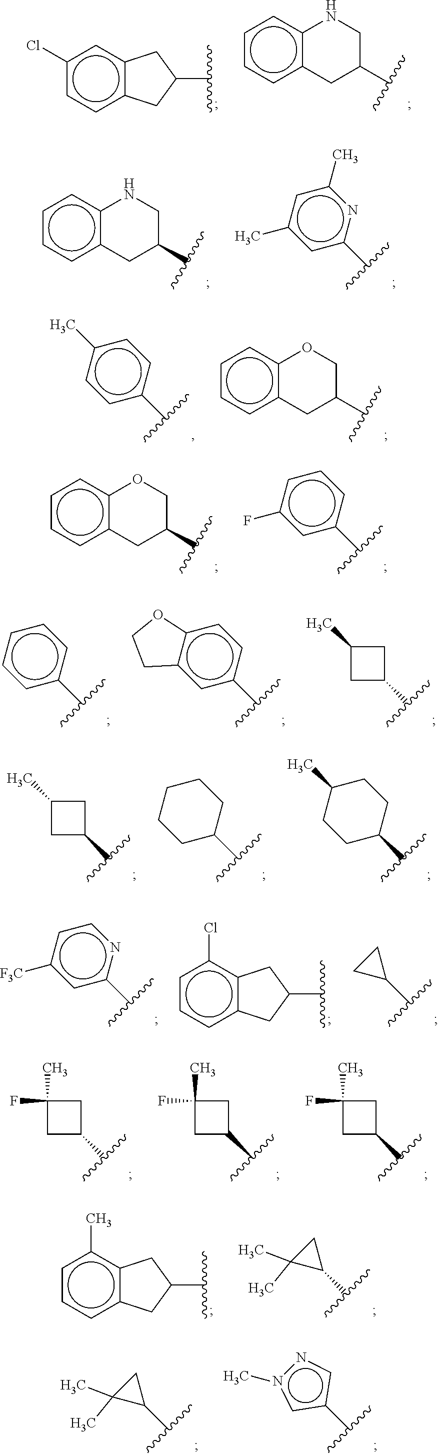

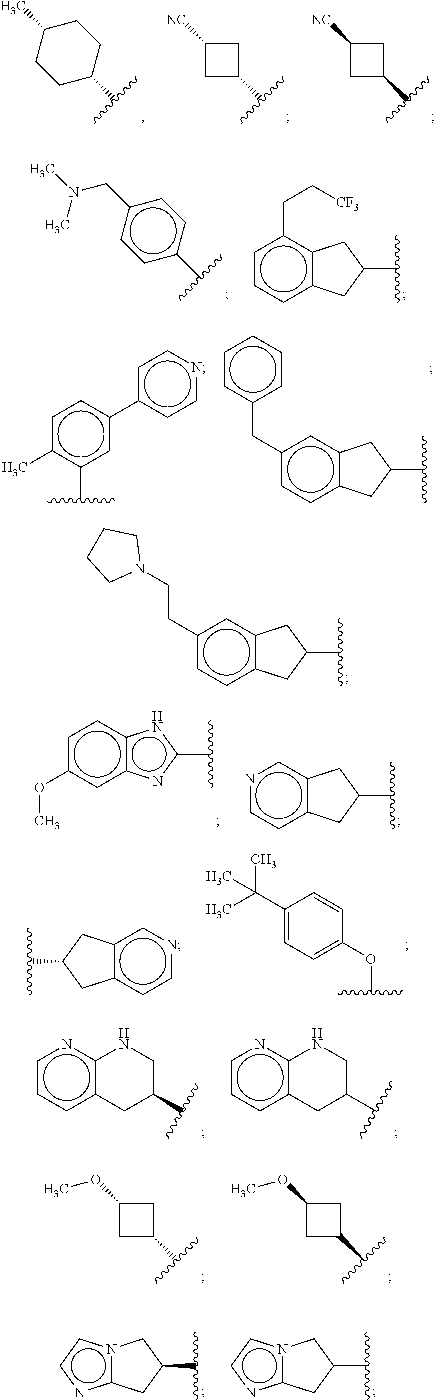

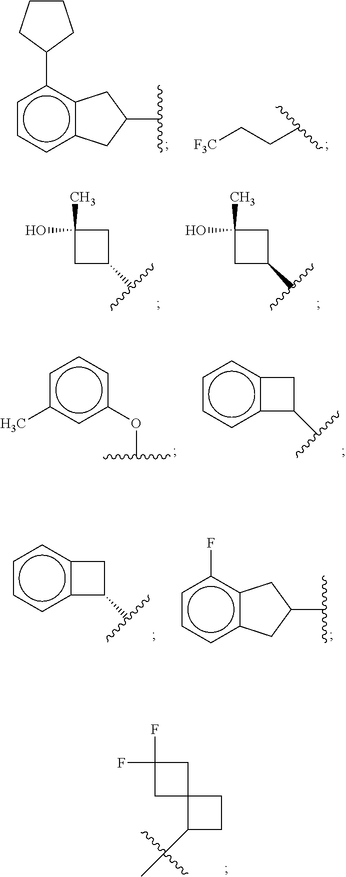

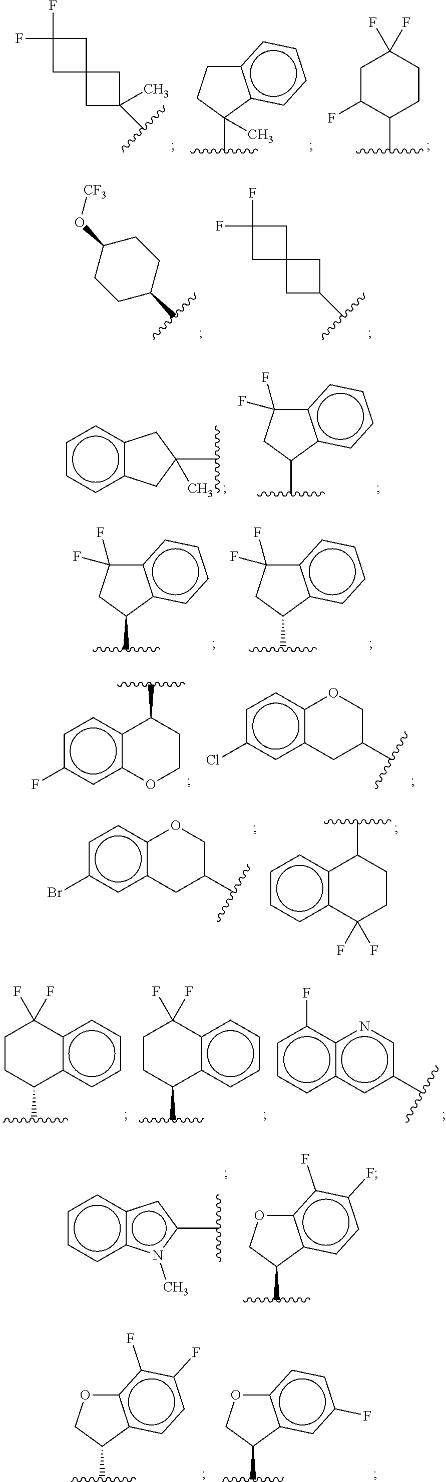

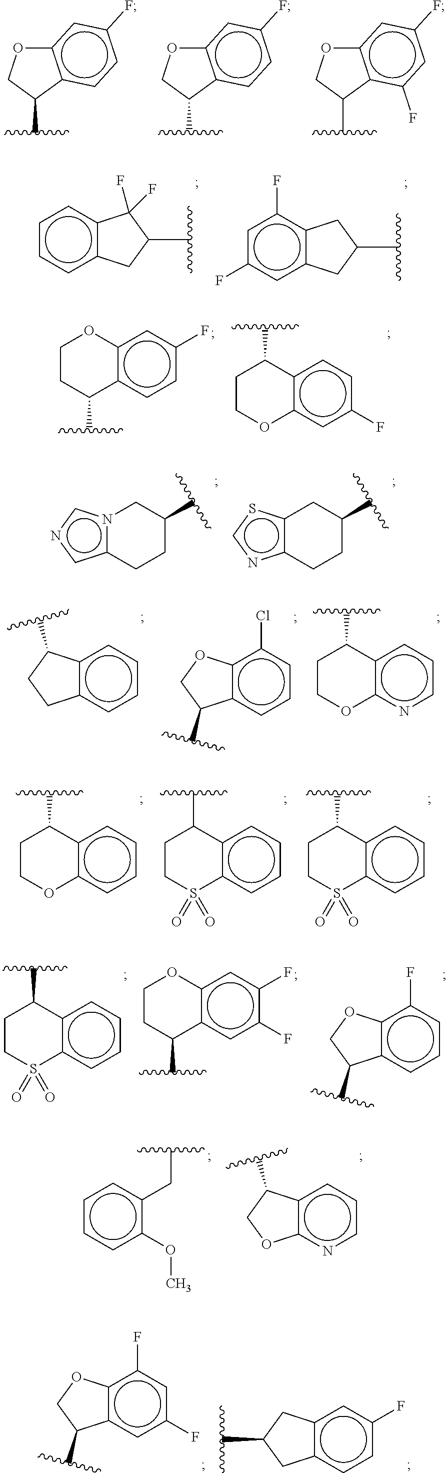

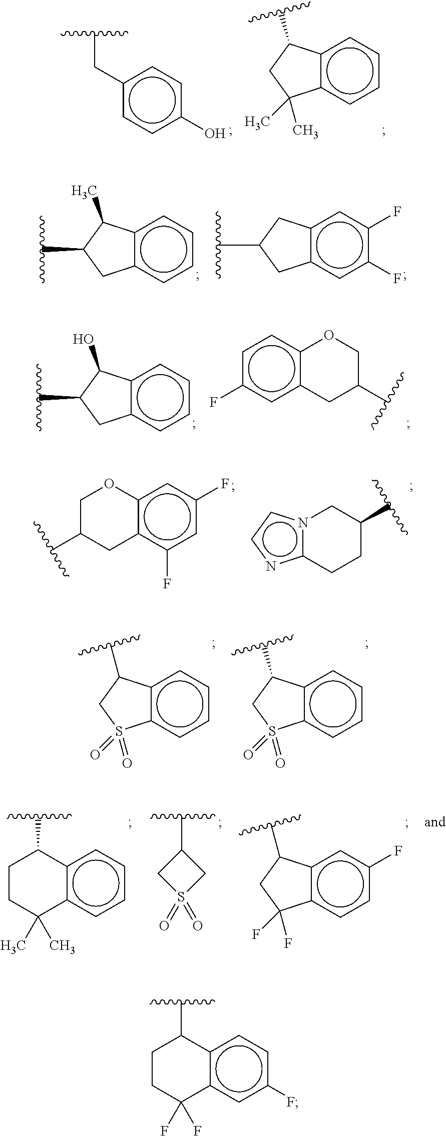

wherein [symbol] indicates the point of attachment to the rest of the compound.

In embodiments of the compound of formula (1),
R¹ is selected from the following groups:

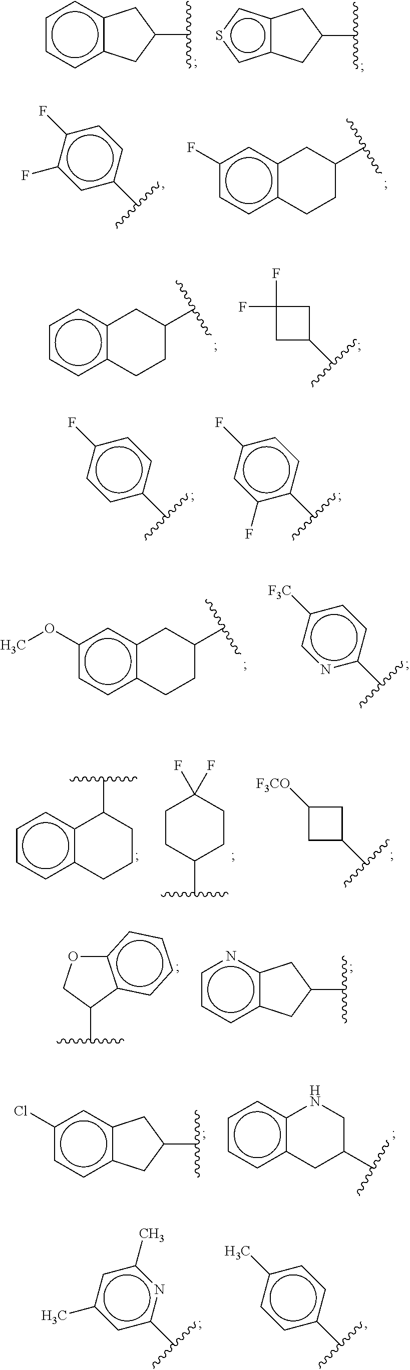

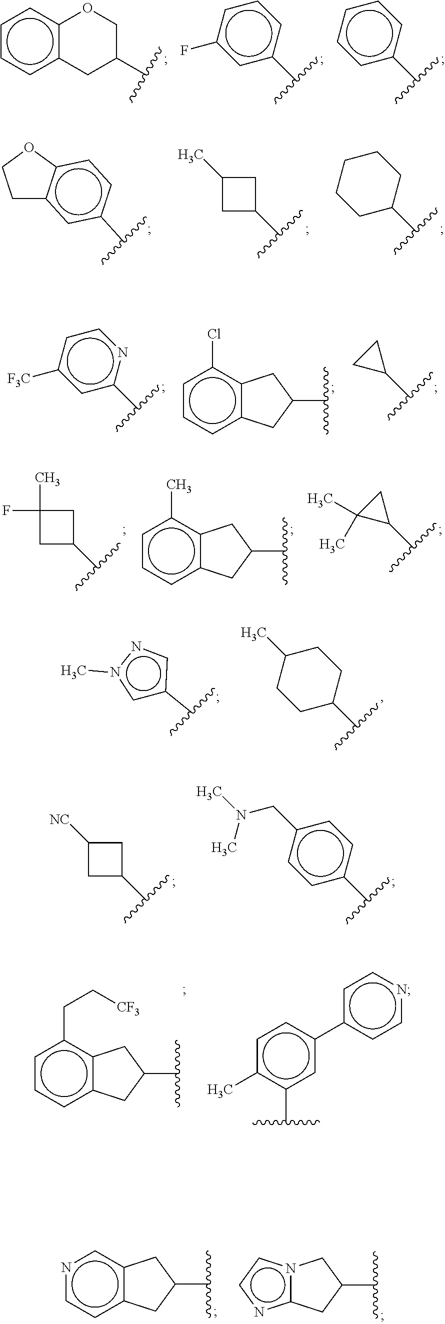

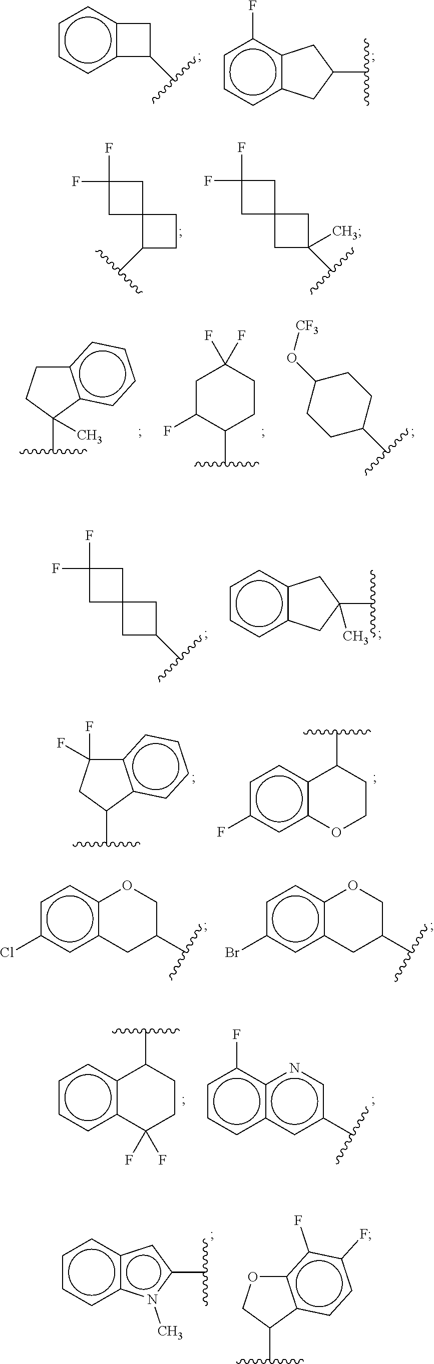

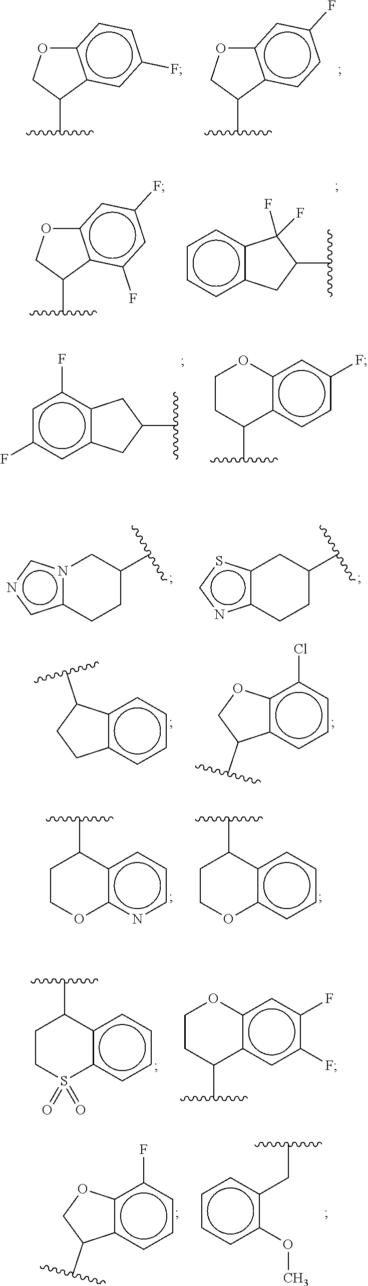

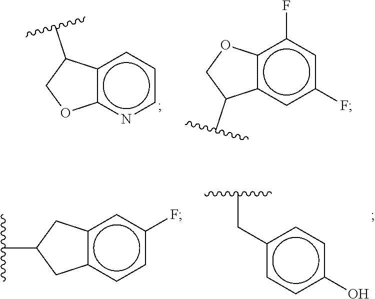

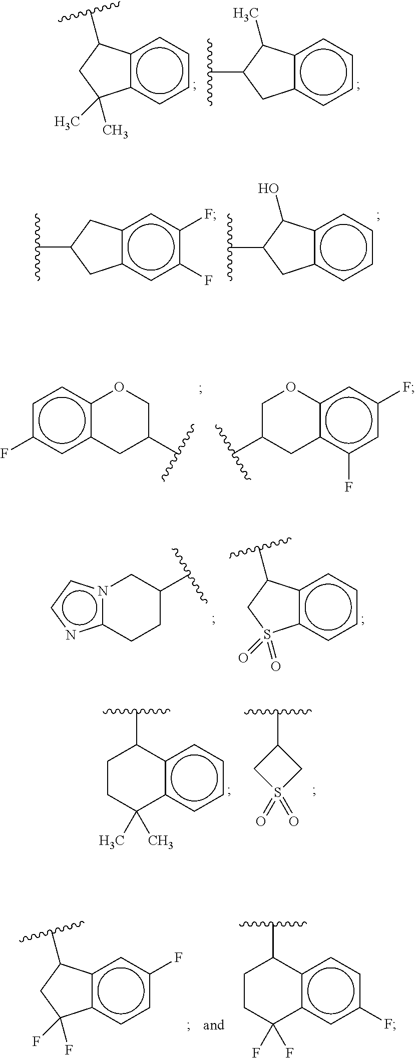

wherein [symbol] indicates the point of attachment to the rest of the compound.

In embodiments of the compound of formula (1),
R¹ is selected from the following groups:

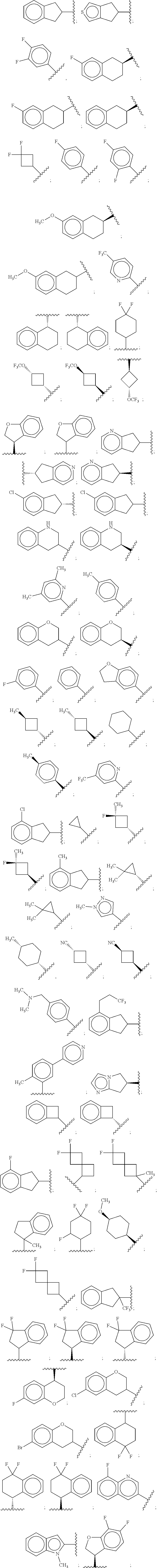

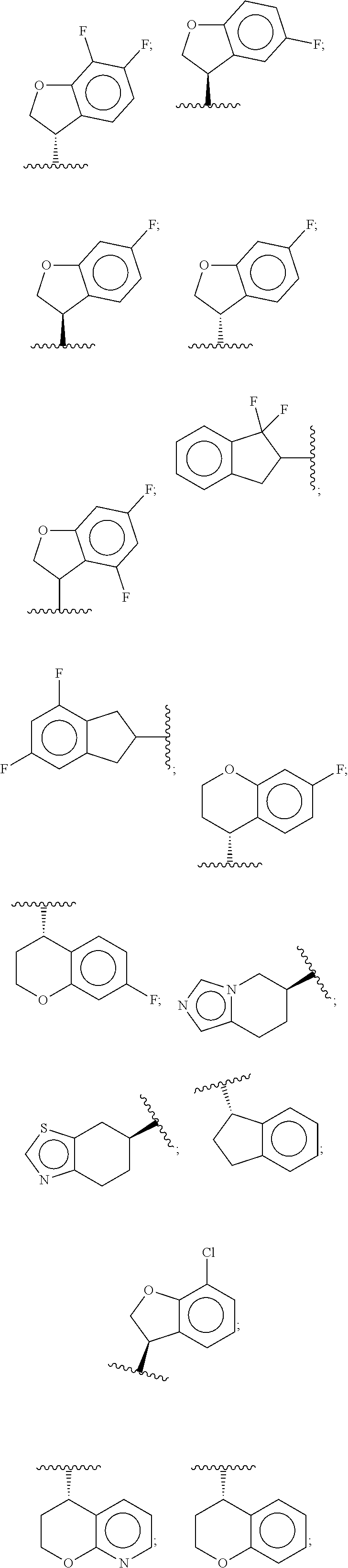

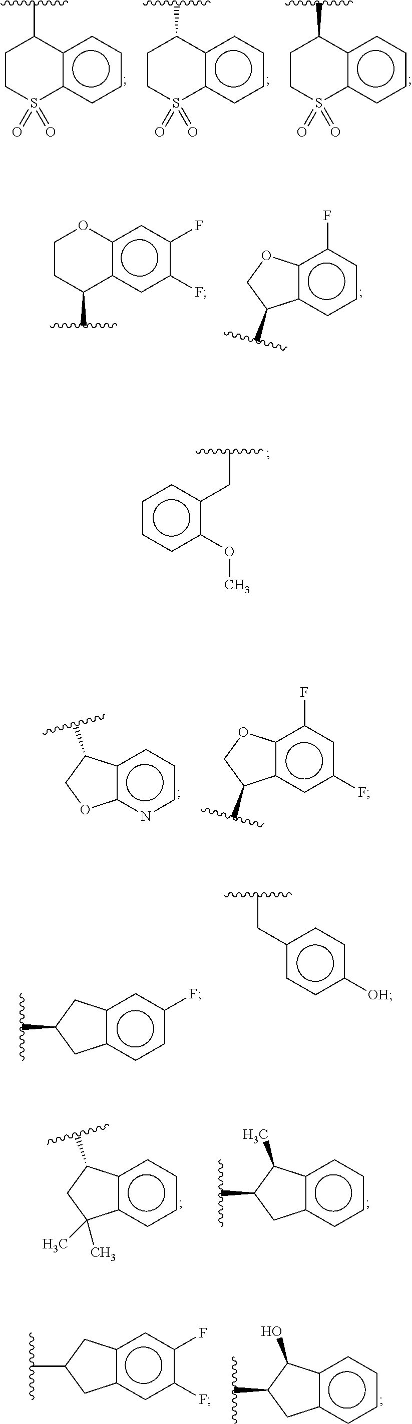

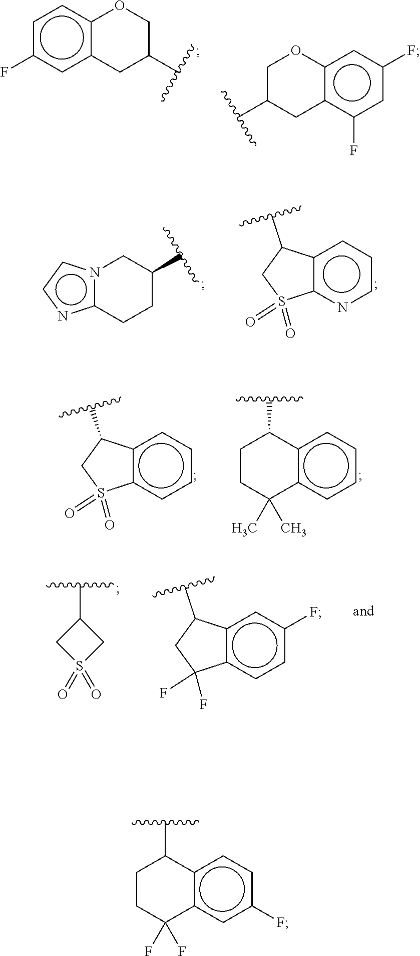

wherein [symbol] indicates the point of attachment to the rest of the compound.

In embodiments of the compound of formula (1),
R¹ is selected from the following groups:

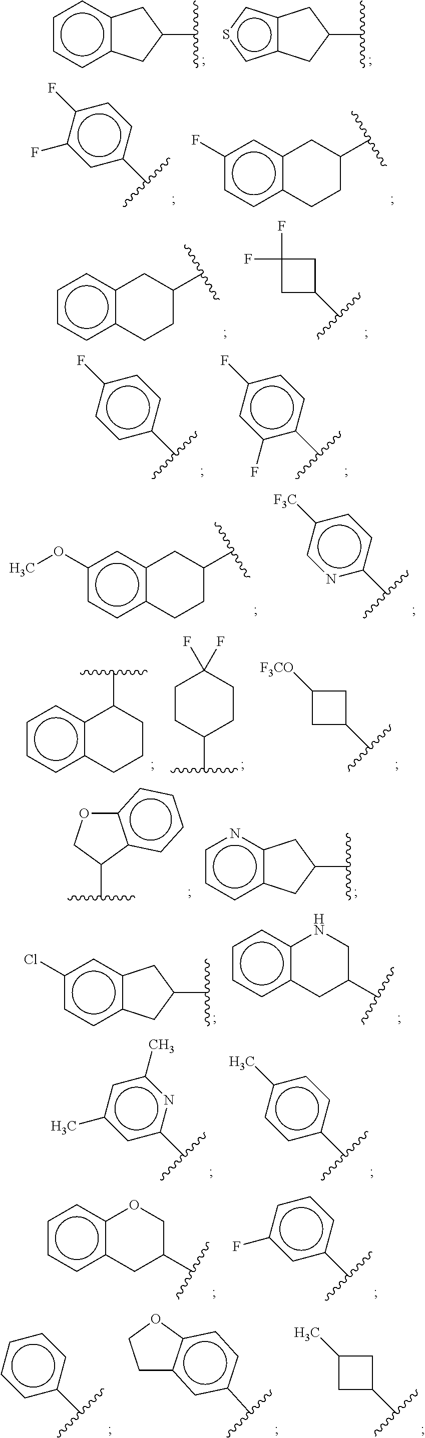

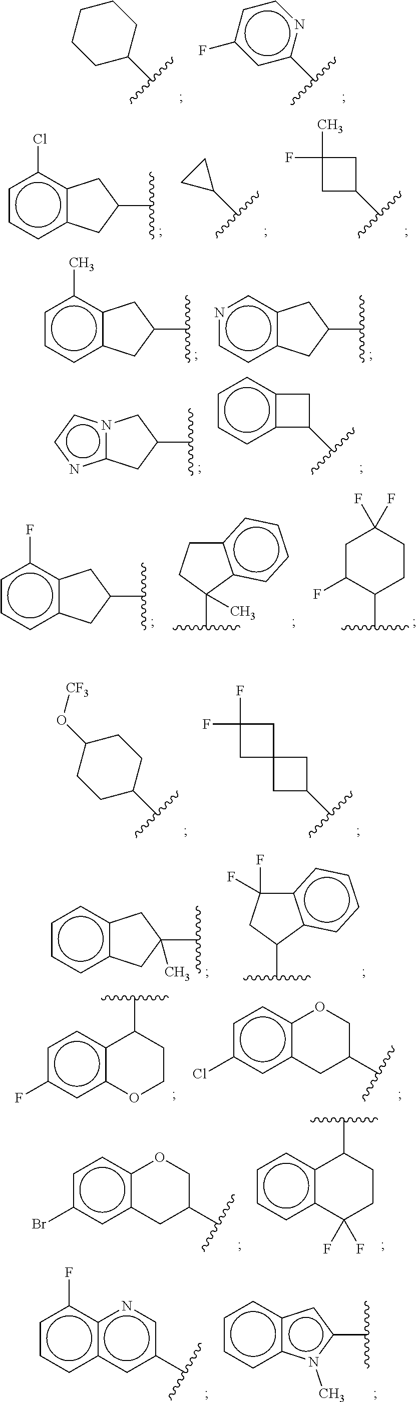

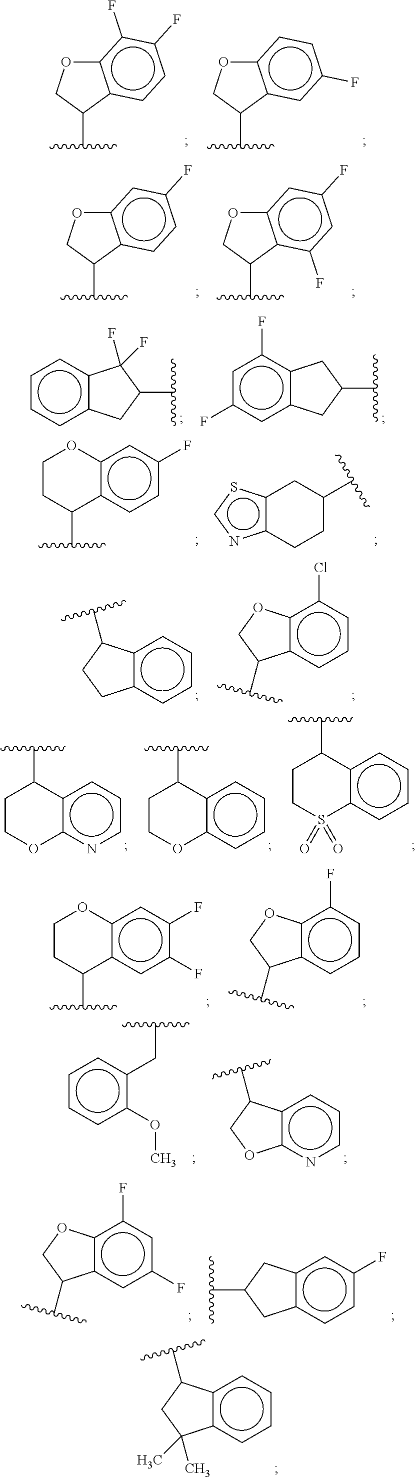

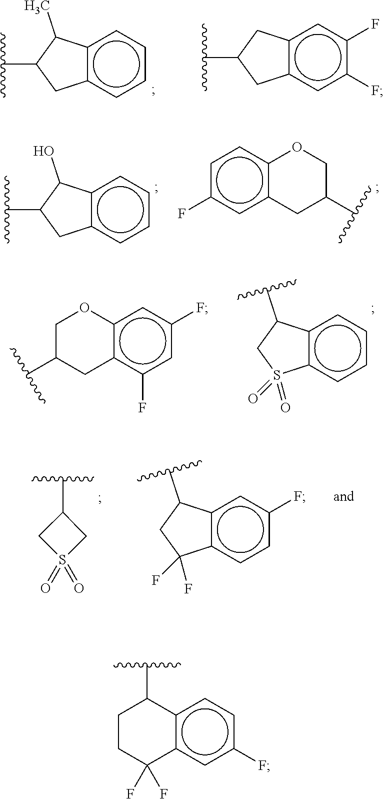

wherein [symbol] indicates the point of attachment to the rest of the compound.

In embodiments of the compound of formula (1),
R¹ is selected from the following groups:

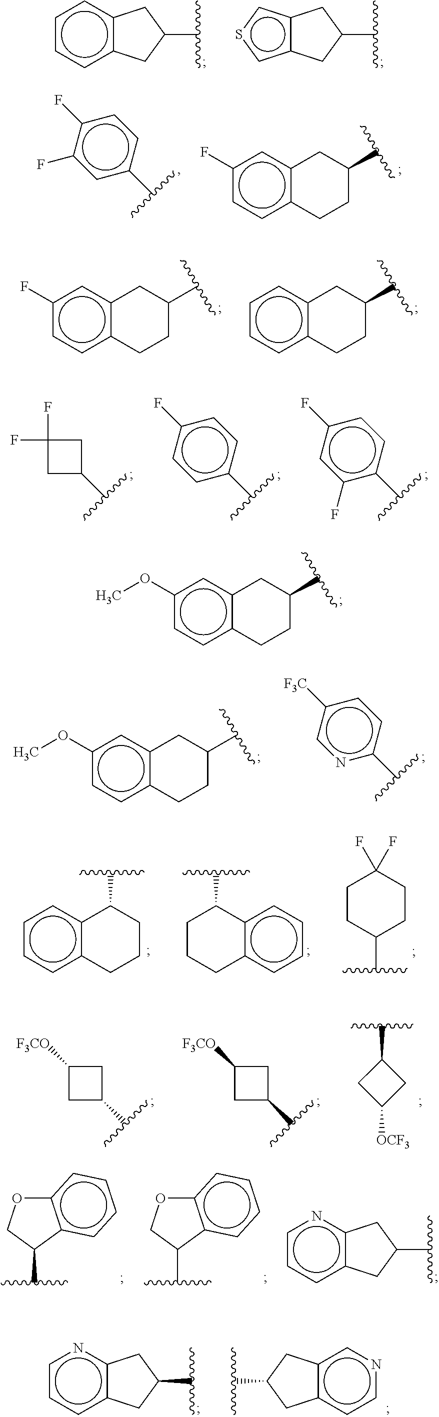

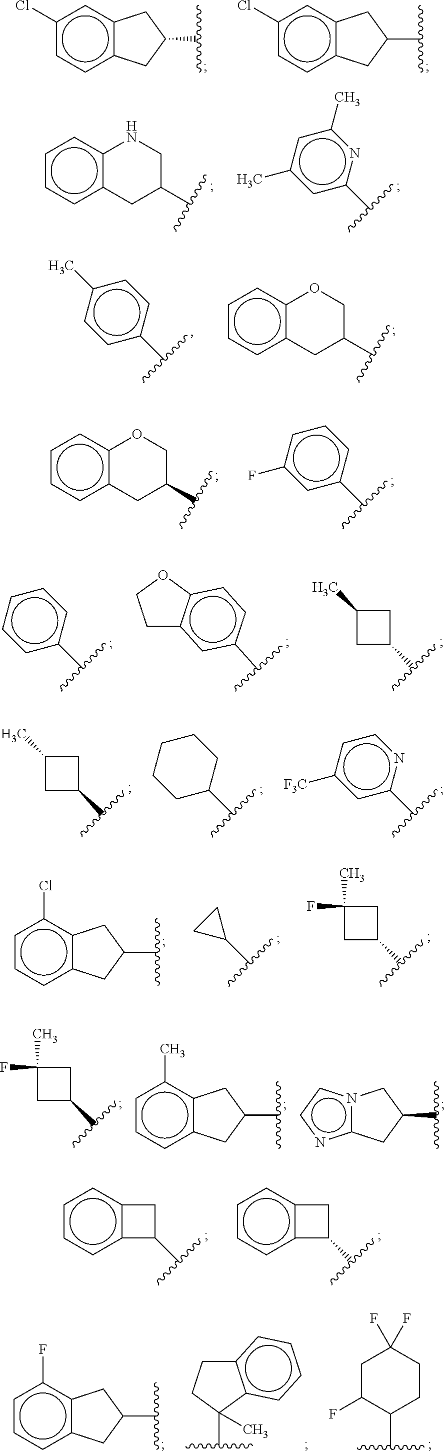

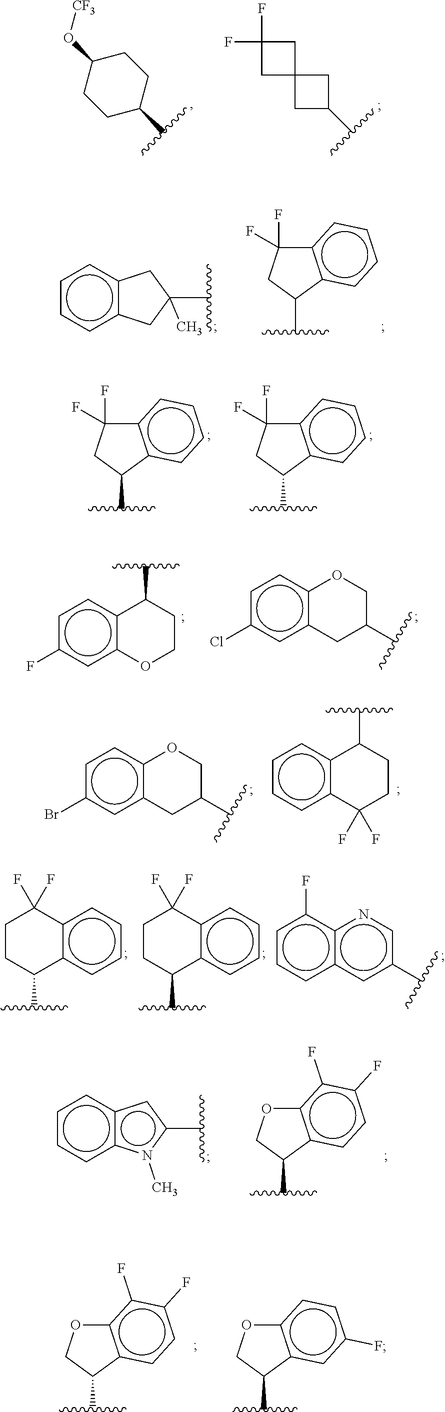

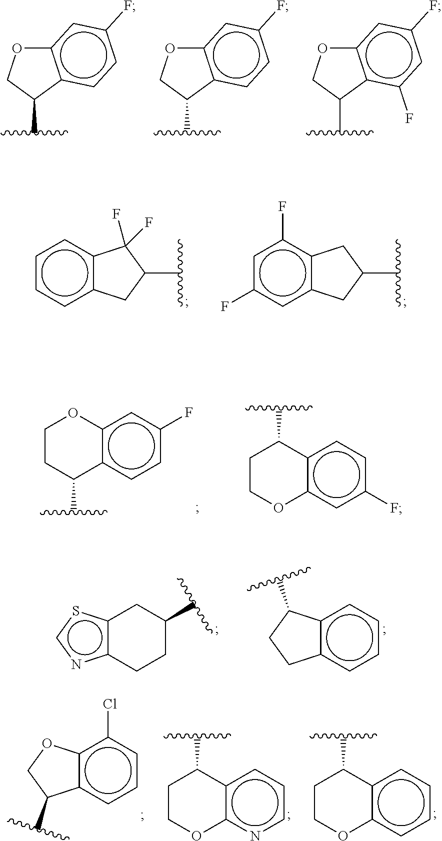

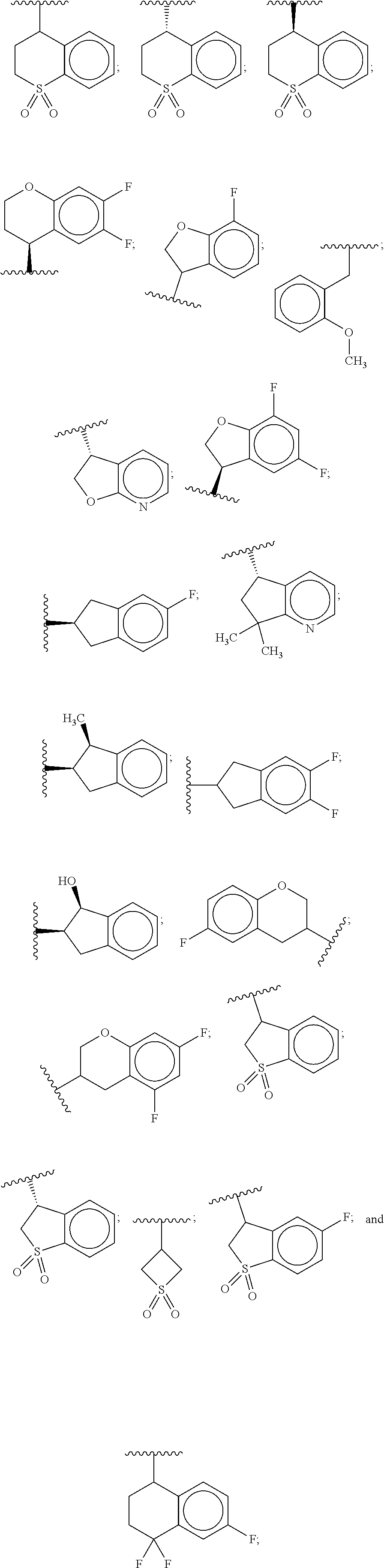

wherein [symbol] indicates the point of attachment to the rest of the compound.

In embodiments of the compound of formula (1),
R¹ is selected from the following groups:

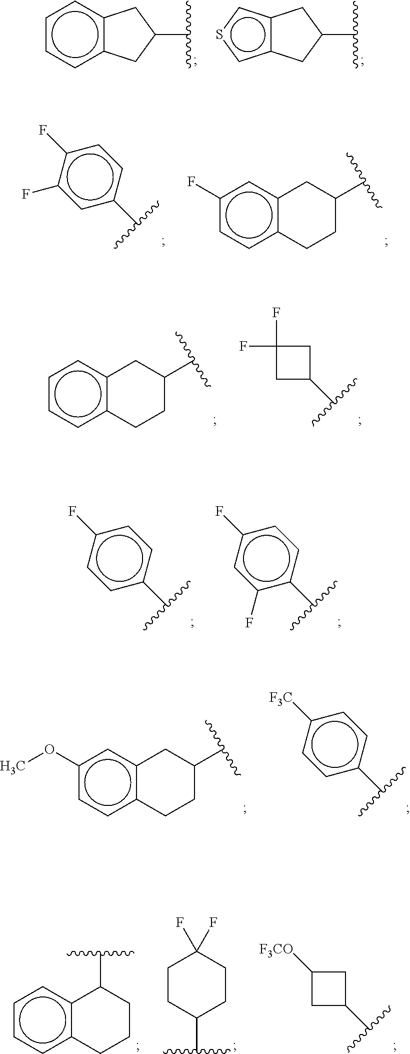

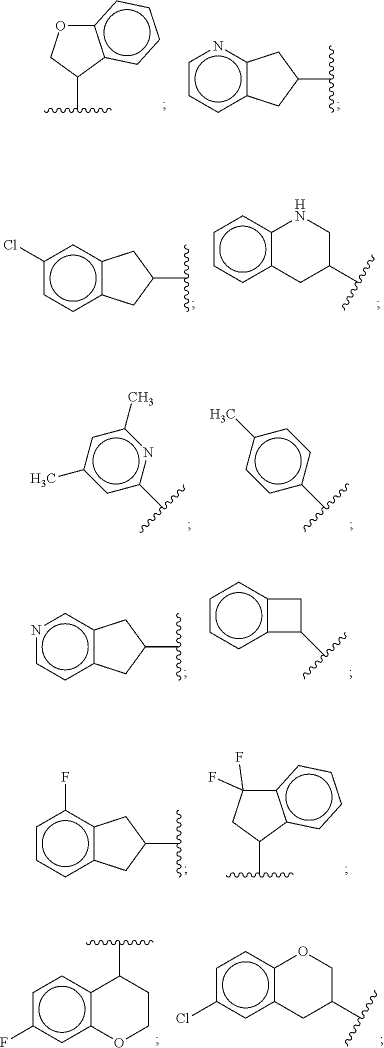

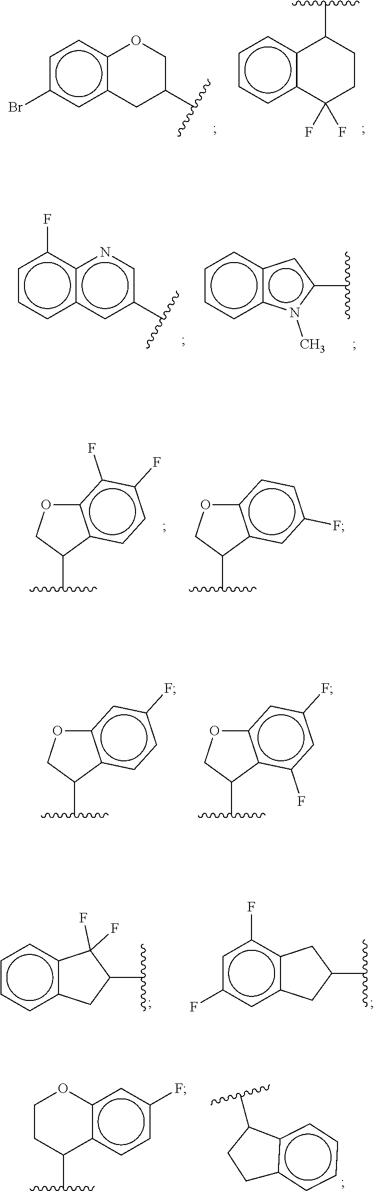

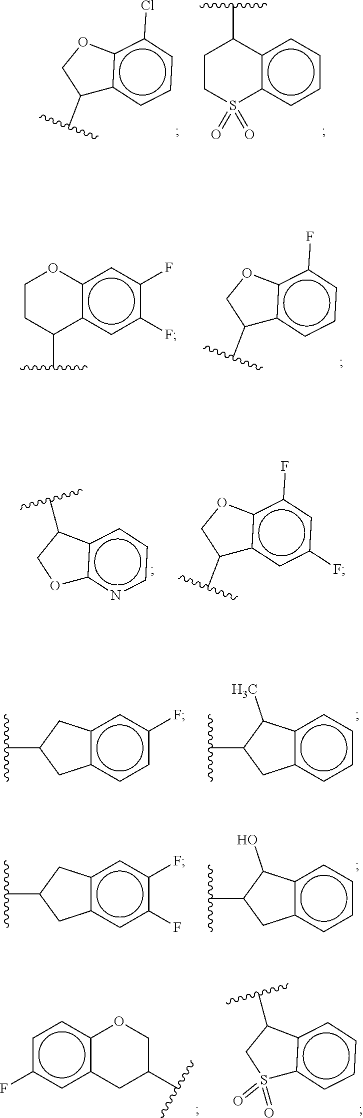

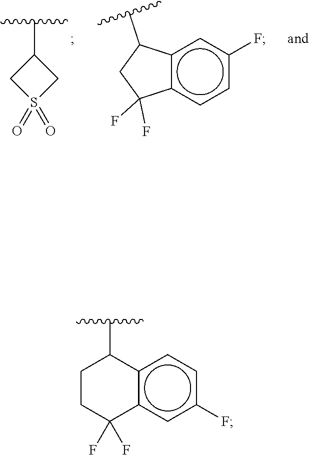

wherein [symbol] indicates the point of attachment to the rest of the compound.

In embodiments of the compound of formula (1),
R¹ is selected from the following groups:

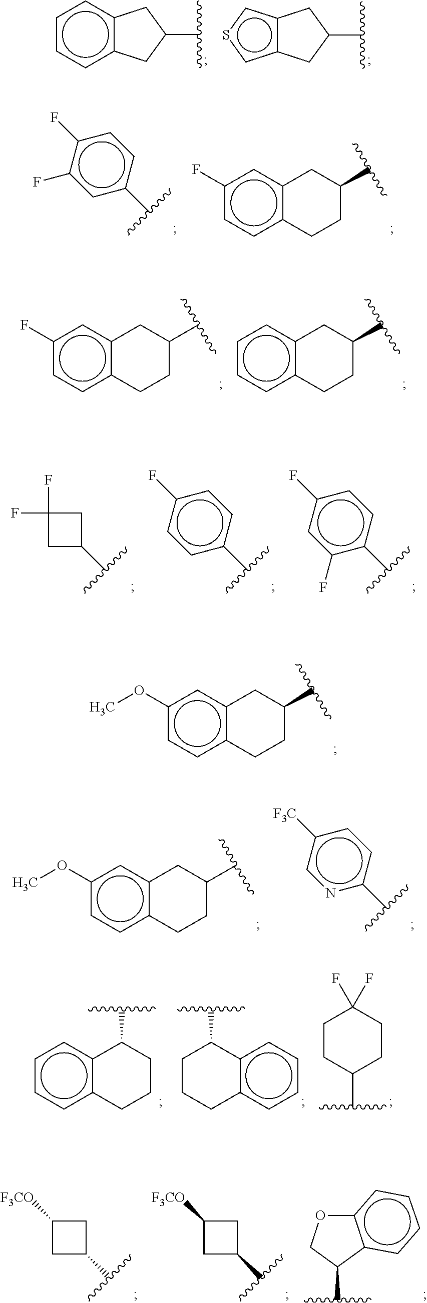

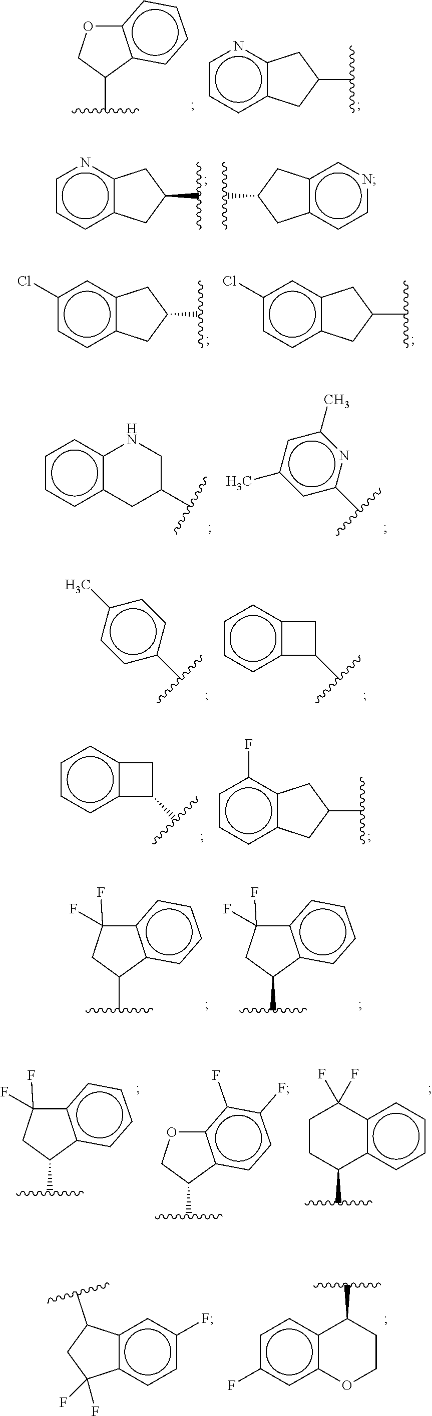

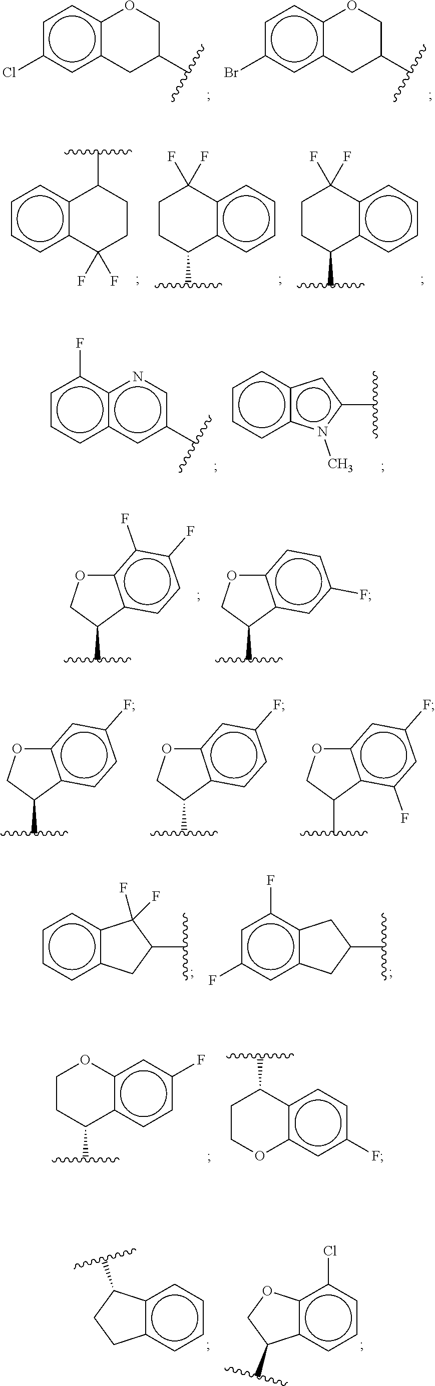

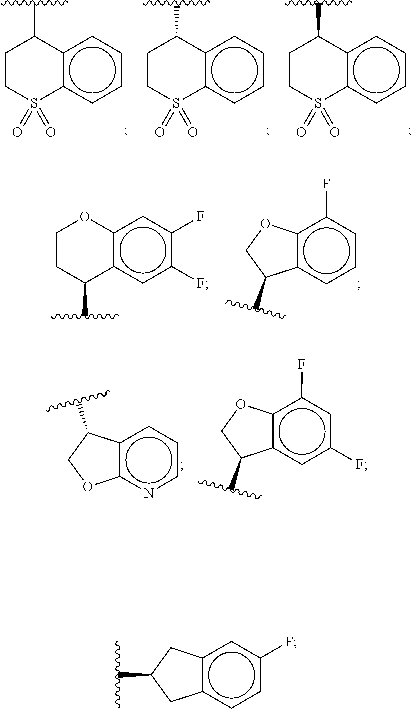

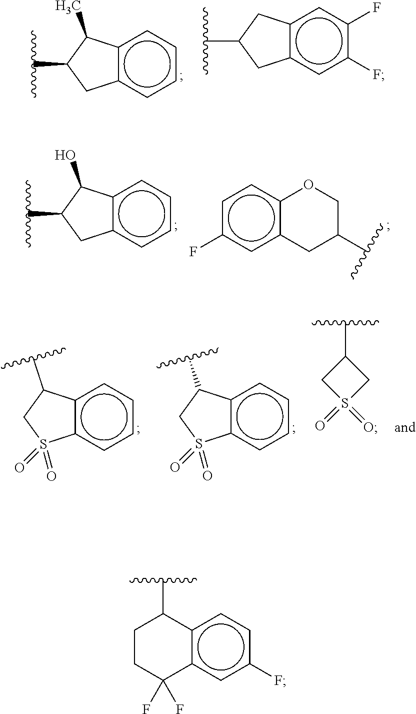

wherein [symbol] indicates the point of attachment to the rest of the compound.

In embodiments of the compound of formula (1),
R¹, R² and L¹ and the nitrogen atom to which R² and L¹ are attached form a heterocycloalkyl, bicyclic heterocycloalkyl, heteroaryl or bicyclic heteroaryl, wherein said heterocycloalkyl, bicyclic heterocycloalkyl, heteroaryl and bicyclic heteroaryl are each optionally substituted by one or more substituents independently selected from halogen, OH, CN, NR^{1a}R^{1b}, alkyl, haloalkyl, alkoxy, haloalkoxy and alkyl-NR^{1a}R^{1b}; and
R^{1a} and R^{1b} are each independently selected from H and alkyl.

In embodiments of the compound of formula (1),
R¹, R² and L¹ and the nitrogen atom to which R² and L¹ are attached form a 3- to 8-membered heterocycloalkyl having one or more heteroatoms selected from N, O and S, 8- to 11-membered bicyclic heterocycloalkyl having one or more heteroatoms selected from N, O and S, 5- or 6-membered heteroaryl having one or more heteroatoms selected from N, O and S, or 8- to 10-membered bicyclic heteroaryl having one or more heteroatoms selected from N, O and S, wherein said heterocycloalkyl, bicyclic heterocycloalkyl, heteroaryl and bicyclic heteroaryl are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy and C₁-C₄ alkyl-NR^{1a}R^{1b}; and
R^{1a} and R^{1b} are each independently selected from H and C₁-C₄ alkyl.

In embodiments of the compound of formula (1),
R¹, R² and L¹ and the nitrogen atom to which R² and L¹ are attached form a 3- to 8-membered heterocycloalkyl having one or more heteroatoms selected from N, O and S, 8- to 11-membered bicyclic heterocycloalkyl having one or more heteroatoms selected from N, O and S, 5- or 6-membered heteroaryl having one or more heteroatoms selected from N, O and S, or 8- to 10-membered bicyclic heteroaryl having one or more heteroatoms selected from N, O and S.

In embodiments of the compound of formula (1),
R¹, R² and L¹ and the nitrogen atom to which R² and L¹ are attached form a 8- to 11-membered bicyclic heterocycloalkyl having one or more heteroatoms selected from N, O and S.

In embodiments of the compound of formula (1),
R¹, R² and L¹ and the nitrogen atom to which R² and L¹ are attached form the following bicyclic heterocycloalkyl group:

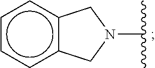

wherein [symbol] indicates the point of attachment to the rest of the compound.

In embodiments of the compound of formula (1),
R² is selected from H, C₁-C₄ alkyl and C₃-C₈ cycloalkyl.

In embodiments of the compound of formula (1),
R² is selected from H and C₁-C₄ alkyl.

In embodiments of the compound of formula (1),
R² is H.

In embodiments of the compound of formula (1),
R³ is selected from H, OH, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkoxy-C₁-C₄ alkyl, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, CN, C₁-C₄ alkyl, C₁-C₄ alkoxy, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl.

In embodiments of the compound of formula (1),
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, CN, C₁-C₄ alkyl, C₁-C₄ alkoxy, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl.

In embodiments of the compound of formula (1),
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, and heteroaryl are each optionally substituted by one or more substituents independently selected from halogen, OH, CN, C₁-C₄ alkyl, C₁-C₄ alkoxy, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl.

In embodiments of the compound of formula (1),
R³ is selected from NR^{3a}R^{3b}, C₁-C₄ alkyl, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, and C₁-C₄ alkyl-S—C₁-C₄ alkyl, wherein said alkyl, cycloalkyl, heterocycloalkyl, and heteroaryl are each optionally substituted by one or more substituents independently selected from halogen, OH, CN, C₁-C₄ alkyl, C₁-C₄ alkoxy, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl.

In embodiments of the compound of formula (1),
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, C₁-C₄ alkyl, and NR^{3c}R^{3d};
R^{3a} is selected from H, C₁-C₄ alkyl, and C₁-C₄ alkyl-NR^{3e}R^{3f};
R^{3b} is selected from H and C₁-C₄ alkyl;
R^{3c} is selected from H and C₁-C₄ alkyl;
R^{3d} is selected from C₁-C₄ alkyl; and
R^{3e} and R^{3f} are each independently selected from C₁-C₄ alkyl.

In embodiments of the compound of formula (1),
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, and heteroaryl are each optionally substituted by one or more substituents independently selected from halogen, OH, C₁-C₄ alkyl, and NR^{3c}R^{3d};
R^{3a} is selected from H, C₁-C₄ alkyl, and C₁-C₄ alkyl-NR^{3e}R^{3f};
R^{3b} is selected from H and C₁-C₄ alkyl;
R^{3c} is selected from H and C₁-C₄ alkyl;
R^{3d} is selected from C₁-C₄ alkyl; and
R^{3e} and R^{3f} are each independently selected from C₁-C₄ alkyl.

In embodiments of the compound of formula (1),
R³ is selected from NR^{3a}R^{3b}, C₁-C₄ alkyl, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, and C₁-C₄ alkyl-S—C₁-C₄ alkyl, wherein said alkyl, cycloalkyl, heterocycloalkyl, and heteroaryl are each optionally substituted by one or more substituents independently selected from halogen, OH, C₁-C₄ alkyl, and NR^{3c}R^{3d};
R^{3a} is selected from H, C₁-C₄ alkyl, and C₁-C₄ alkyl-NR^{3e}R^{3f};
R^{3b} is selected from H and C₁-C₄ alkyl;
R^{3c} is selected from H and C₁-C₄ alkyl;
R^{3d} is selected from C₁-C₄ alkyl; and
R^{3e} and R^{3f} are each independently selected from C₁-C₄ alkyl.

In embodiments of the compound of formula (1),
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH and NR^{3c}R^{3d};
R^{3a} is selected from H and C₁-C₄ alkyl;
R^{3b} is selected from C₁-C₄ alkyl;
R^{3c} is selected from H and C₁-C₄ alkyl; and
R^{3d} is selected from C₁-C₄ alkyl.

In embodiments of the compound of formula (1),
wherein
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₃-C₈ cycloalkoxy-C₁-C₄ alkyl, C₃-C₈ cycloalkyl-C₁-C₄ alkoxy, C₁-C₄ haloalkoxy-C₁-C₄ alkyl, C₁-C₄ alkyl-S—C₁-C₄ alkyl, and C₁-C₄ alkyl-SO₂-C₁-C₄ alkyl, wherein said alkyl, cycloalkyl, heterocycloalkyl, and heteroaryl are each optionally substituted by one or more substituents independently selected from halogen, OH and NR^{3c}R^{3d};
R^{3a} is selected from H and C₁-C₄ alkyl;
R^{3b} is selected from C₁-C₄ alkyl;
R^{3c} is selected from H and C₁-C₄ alkyl; and
R^{3d} is selected from C₁-C₄ alkyl.

In embodiments of the compound of formula (1),
R³ is selected from NR^{3a}R^{3b}, C₁-C₄ alkyl, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₃-C₈ cycloalkoxy-C₁-C₄ alkyl, C₃-C₈ cycloalkyl-C₁-C₄ alkoxy, C₁-C₄ haloalkoxy-C₁-C₄ alkyl, and C₁-C₄ alkyl-S—C₁-C₄ alkyl, wherein said alkyl, cycloalkyl, heterocycloalkyl, and heteroaryl are each optionally substituted by one or more substituents independently selected from halogen, OH and NR^{3c}R^{3d};
R^{3a} is selected from H and C₁-C₄ alkyl;
R^{3b} is selected from C₁-C₄ alkyl;
R^{3c} is selected from H and C₁-C₄ alkyl; and
R^{3d} is selected from C₁-C₄ alkyl.

In embodiments of the compound of formula (1),
R³ is selected from H, CN, NR^{3a}R^{3b}, C₁-C₄ alkyl optionally substituted by OH, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₃-C₈ cycloalkoxy-C₁-C₄ alkyl, C₃-C₈ cycloalkyl-C₁-C₄ alkoxy, C₁-C₄ haloalkoxy-C₁-C₄ alkyl, C₁-C₄ alkyl-S—C₁-C₄ alkyl, and C₁-C₄ alkyl-SO₂—C₁-C₄ alkyl;
R^{3a} is H; and
R^{3b} is selected from C₁-C₄ alkyl.

In embodiments of the compound of formula (1),
R³ is selected from NR^{3a}R^{3b}, C₁-C₄ alkyl substituted by one or more substituents independently selected from OH and NR^{3c}R^{3d}, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₃-C₈ cycloalkoxy-C₁-C₄ alkyl, C₃-C₈ cycloalkyl-C₁-C₄ alkoxy, C₁-C₄ haloalkoxy-C₁-C₄ alkyl, and C₁-C₄ alkyl-S—C₁-C₄ alkyl;
R^{3a} is H;
R^{3b} is selected from C₁-C₄ alkyl.

In embodiments of the compound of formula (1),
R³ is selected from:
   H;
   CN;
   NR^{3a}R^{3b};
   C₁-C₄ alkyl optionally substituted by one of more substituents selected from OH and NR^{3c}R^{3d};
   C₃-C₈ cycloalkyl;
   3- to 8-membered heterocycloalkyl having one or more heteroatoms selected from N, O and S, optionally substituted by one or more C₁-C₄ alkyl;
   phenyl optionally substituted by one or more halogen;
   5- or 6-membered heteroaryl having one or more heteroatoms selected from N, O and S;
   C₁-C₄ alkoxy-C₁-C₄ alkyl;
   C₁-C₄ haloalkoxy-C₁-C₄ alkyl;
   C₃-C₈ cycloalkoxy-C₁-C₄ alkyl;
   C₃-C₈ cycloalkyl-C₁-C₄ alkoxy;
   C₁-C₄ alkyl-S—C₁-C₄ alkyl; and
   C₁-C₄ alkyl-SO₂-C₁-C₄ alkyl;
wherein
R^{3a} is selected from H, C₁-C₄ alkyl, and C₁-C₄ alkyl-NR^{3e}R^{3f};
R^{3b} is selected from H and C₁-C₄ alkyl;
R^{3c} is selected from H and C₁-C₄ alkyl;
R^{3d} is selected from C₁-C₄ alkyl; and
R^{3e} and R^{3f} are each independently selected from C₁-C₄ alkyl.

In embodiments of the compound of formula (1),
R³ is selected from:
   H;
   CN;
   NR^{3a}R^{3b};
   C₁-C₄ alkyl optionally substituted by one or more substituents independently selected from OH and NR^{3c}R^{3d};
   C₃-C₈ cycloalkyl;
   3- to 8-membered heterocycloalkyl having one or more heteroatoms selected from N, O and S;
   phenyl substituted by one or more halogen;
   5- or 6-membered heteroaryl having one or more heteroatoms selected from N, O and S;
   C₁-C₄ alkoxy-C₁-C₄ alkyl;
   C₃-C₈ cycloalkyl-C₁-C₄ alkoxy;
   C₃-C₈ cycloalkoxy-C₁-C₄ alkyl;
   C₁-C₄ haloalkoxy-C₁-C₄ alkyl;
   C₁-C₄ alkyl-S—C₁-C₄ alkyl; and
   C₁-C₄ alkyl-SO₂-C₁-C₄ alkyl;
wherein
R^{3a} is selected from H and C₁-C₄ alkyl;
R^{3b} is selected from C₁-C₄ alkyl;
R^{3c} is selected from H and C₁-C₄ alkyl; and
R^{3d} is selected from C₁-C₄ alkyl.

In embodiments of the compound of formula (1),
R³ is selected from:
   H;
   CN;
   NR^{3a}R^{3b};
   C₁-C₄ alkyl optionally substituted by OH;
   C₃-C₈ cycloalkyl;
   3- to 8-membered heterocycloalkyl having one or more heteroatoms selected from N, O and S;
   5- or 6-membered heteroaryl having one or more heteroatoms selected from N, O and S;
   C₁-C₄ alkoxy-C₁-C₄ alkyl;
   C₃-C₈ cycloalkyl-C₁-C₄ alkoxy;
   C₃-C₈ cycloalkoxy-C₁-C₄ alkyl;
   C₁-C₄ haloalkoxy-C₁-C₄ alkyl;
   C₁-C₄ alkyl-S—C₁-C₄ alkyl; and
   C₁-C₄ alkyl-SO₂-C₁-C₄ alkyl;
wherein
R^{3a} is H; and
R^{3b} is selected from C₁-C₄ alkyl.

Clause 89. The compound of any preceding clause, wherein
R³ is selected from:
   NR^{3a}R^{3b};
   C₁-C₄ alkyl optionally substituted by OH;
   C₃-C₈ cycloalkyl;
   3- to 8-membered heterocycloalkyl having one or more heteroatoms selected from N, O and S;
   5- or 6-membered heteroaryl having one or more heteroatoms selected from N, O and S;
   C₁-C₄ alkoxy-C₁-C₄ alkyl;
   C₃-C₈ cycloalkyl-C₁-C₄ alkoxy;
   C₃-C₈ cycloalkoxy-C₁-C₄ alkyl;
   C₁-C₄ haloalkoxy-C₁-C₄ alkyl; and
   C₁-C₄ alkyl-S—C₁-C₄ alkyl;
wherein
R^{3a} is H; and
R^{3b} is selected from C₁-C₄ alkyl.

In embodiments of the compound of formula (1),
R³ is selected from C₁-C₄ alkoxy-C₁-C₄ alkyl.

In embodiments of the compound of formula (1),
wherein R³ is selected from methoxymethyl.

In embodiments of the compound of formula (1),
R³ is selected from the following groups:

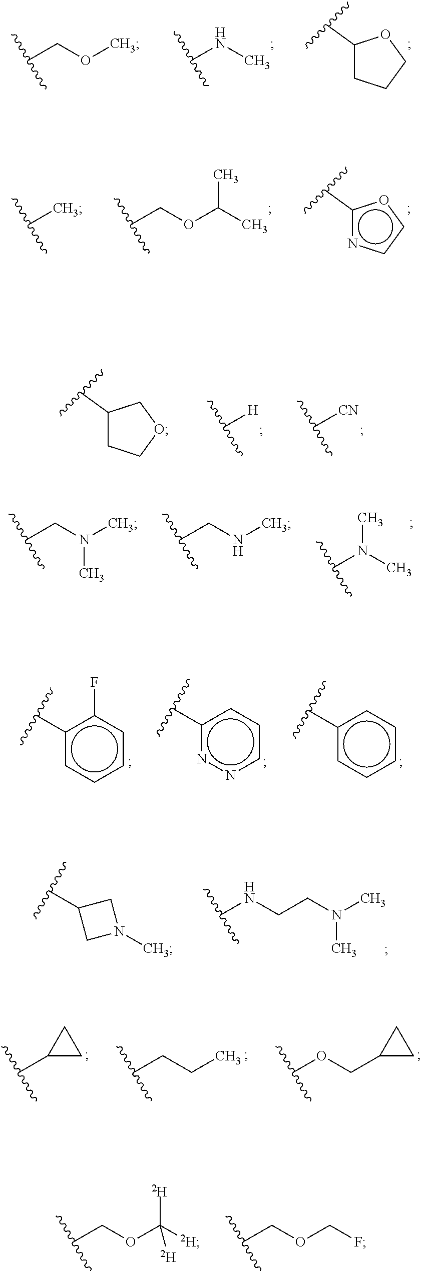

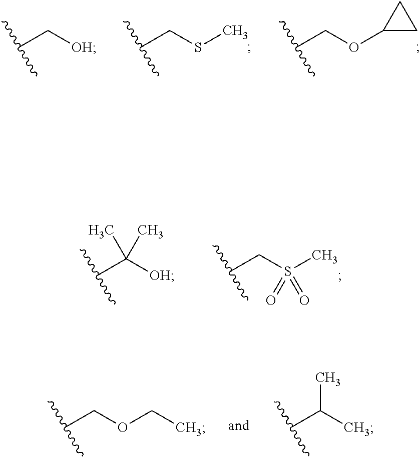

wherein [symbol] indicates the point of attachment to the rest of the compound.

In embodiments of the compound of formula (1),
R³ is selected from the following groups:

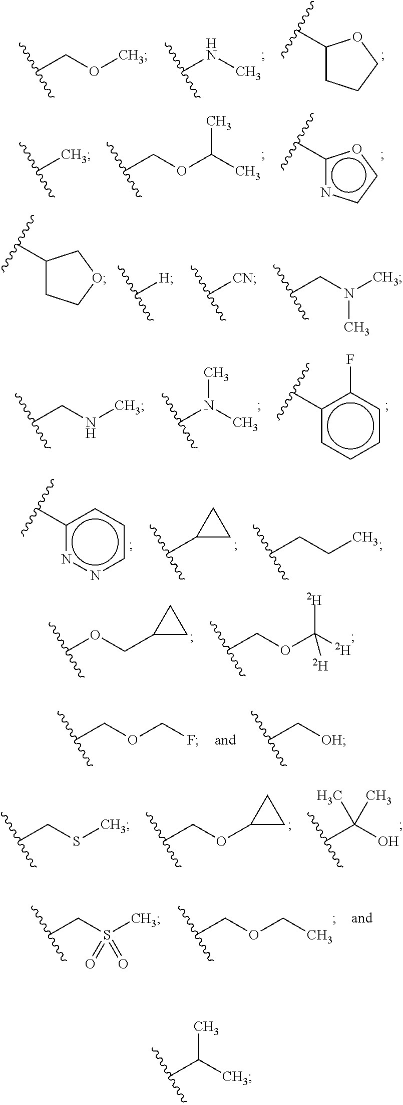

wherein [symbol] indicates the point of attachment to the rest of the compound.

In embodiments of the compound of formula (1),
R³ is selected from the following groups:

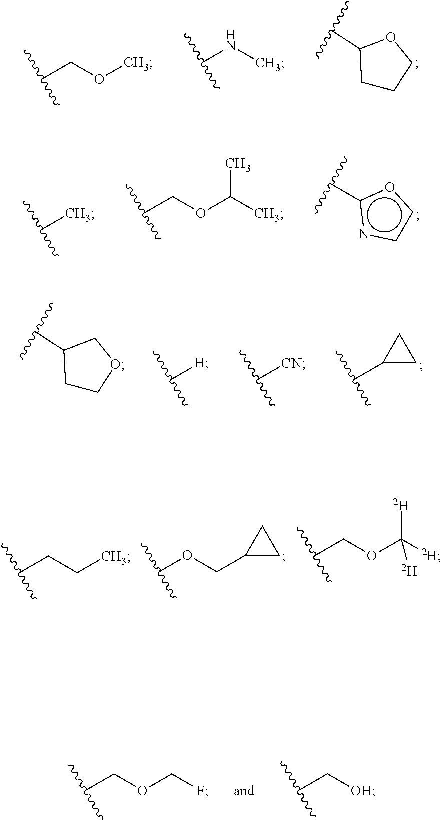

wherein [symbol] indicates the point of attachment to the rest of the compound.

In embodiments of the compound of formula (1),
R³ is selected from the following groups:

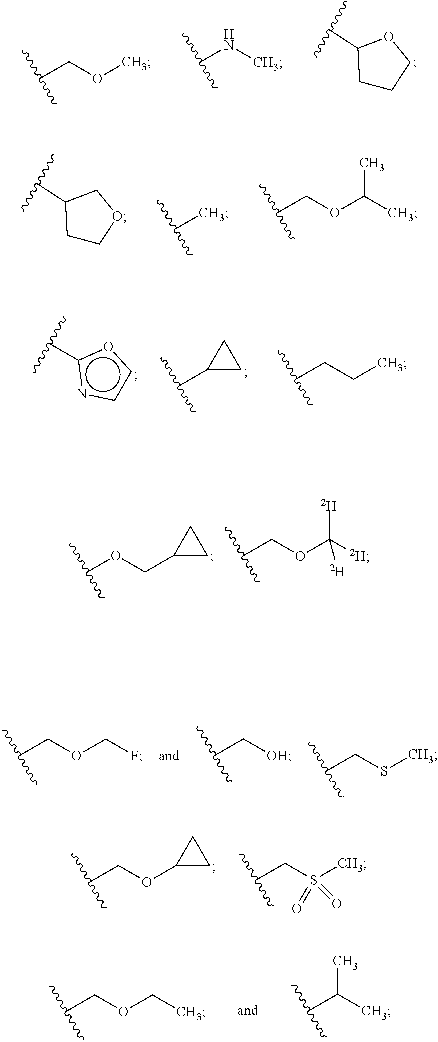

wherein [symbol] indicates the point of attachment to the rest of the compound.

In embodiments of the compound of formula (1),
R⁴ is selected from C(O)OH, C(O)NR^{4a}R^{4b}, C(S)NR^{4a}R^{4b} and 5- or 6-membered heteroaryl having one or more heteroatoms selected from N, O and S; and
R^{4a} and R^{4b} are each independently selected from H and alkyl.

In embodiments of the compound of formula (1),
R⁴ is selected from C(O)NR^{4a}R^{4b} and 5- or 6-membered heteroaryl having one or more heteroatoms selected from N, O and S; and
R^{4a} and R^{4b} are each independently selected from H and alkyl.

In embodiments of the compound of formula (1),
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl; and
R^{4b} is H.

In embodiments of the compound of formula (1),
R⁴ is selected from C(O)OH, C(O)NR^{4a}R^{4b}, and C(S)NR^{4a}R^{4b};
R^{4a} is selected from H and C₁-C₄ alkyl; and
R^{4b} is H.

In embodiments of the compound of formula (1),
R⁴ is selected from C(O)OH, C(O)NR^{4a}R^{4b} and C(S)NR^{4a}R^{4b}; and
R^{4a} and R^{4b} are each H.

In embodiments of the compound of formula (1),
R⁴ is C(O)NR^{4a}R^{4b}; and
R^{4a} and R^{4b} are each H.

In embodiments of the compound of formula (1),
R⁴ is selected from the following groups:

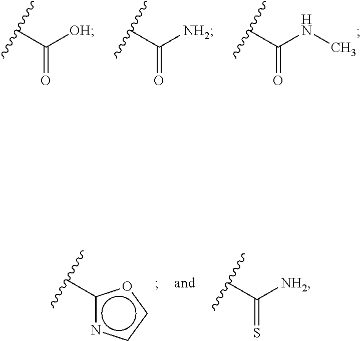

wherein [symbol] indicates the point of attachment to the rest of the compound.

In embodiments of the compound of formula (1),
R⁴ is selected from the following groups:

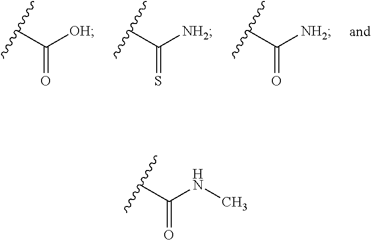

wherein [symbol] indicates the point of attachment to the rest of the compound.

In embodiments of the compound of formula (1),
R⁴ is selected from the following groups:

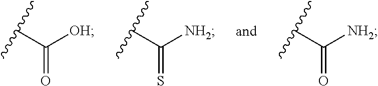

wherein [symbol] indicates the point of attachment to the rest of the compound.

In embodiments of the compound of formula (1),
R⁴ is

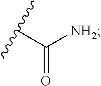 [SMILES: *C(N)=O]

wherein [symbol] indicates the point of attachment to the rest of the compound.

In embodiments of the compound of formula (1),
R⁵ and R⁶ are each independently selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S; or
R⁵ and R⁶ together with the carbon atoms to which they are attached form a 5- or 6-membered heterocycle having one or more heteroatoms selected from N, O and S.

In embodiments of the compound of formula (1),
R⁵ and R⁶ are each independently selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S; or R⁵ and R⁶ together with the carbon atoms to which they are attached form a 5- or 6-membered heterocycle having one or more heteroatoms selected from N and O.

In embodiments of the compound of formula (1),
R⁵ is selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl, and 5- or 6-membered heteroaryl having one or more heteroatoms selected from N, O and S.

In embodiments of the compound of formula (1),
R⁵ is selected from H, C₁-C₄ alkyl, and 5- or 6-membered heteroaryl having one or more heteroatoms selected from N, O and S.

In embodiments of the compound of formula (1),
R⁵ is selected from H and 5- or 6-membered heteroaryl having one or more heteroatoms selected from N, O and S.

In embodiments of the compound of formula (1),
R⁵ is selected from the following groups:

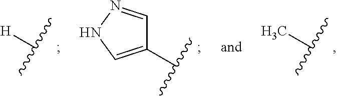

wherein [symbol] indicates the point of attachment to the rest of the compound.

In embodiments of the compound of formula (1),
R⁵ is selected from the following groups:

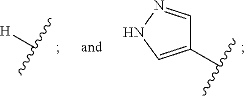

wherein [symbol] indicates the point of attachment to the rest of the compound.

In embodiments of the compound of formula (1),

R⁵ is H.

In embodiments of the compound of formula (1),
R⁶ is selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl, and 5- or 6-membered heteroaryl having one or more heteroatoms selected from N, O and S.

In embodiments of the compound of formula (1),
R⁶ is selected from H, C₁-C₄ alkyl, C₁-C₄ alkoxy, and C₁-C₄ alkoxy-C₁-C₄ alkyl.

In embodiments of the compound of formula (1),
R⁶ is selected from H and C₁-C₄ alkyl.

In embodiments of the compound of formula (1),
R⁶ is selected from the following groups:

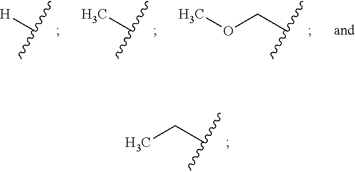

wherein [symbol] indicates the point of attachment to the rest of the compound.

In embodiments of the compound of formula (1),
R⁶ is H or methyl.

In embodiments of the compound of formula (1),
R⁶ is H.

In embodiments of the compound of formula (1),
R⁵ and R⁶ together with the carbon atoms to which they are attached form a 5- or 6-membered heterocycle having one or more heteroatoms selected from N, O and S.

In embodiments of the compound of formula (1),
R⁵ and R⁶ together with the carbon atoms to which they are attached form a 5- or 6-membered heterocycle having one or more heteroatoms selected from N and O.

In embodiments of the compound of formula (1),
R⁵ and R⁶ together with the carbon atoms to which they are attached form a 5-membered heterocycle containing one O atom.

In embodiments of the compound of formula (1),
R⁷, R⁸, R⁹, R¹⁰, R¹¹, and R¹² are each H.

Also disclosed herein is a compound according to the invention having the structural formula (2):

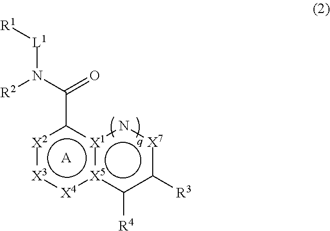

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein:
X¹, X², X³, X⁴, and X⁵ are as defined for compounds of formula (1);
X⁷ is selected from CR¹⁰, O, and N; and
q, L¹, R¹, R², R³, R⁴, and R¹⁰ are as defined in any preceding clause;
wherein when q is 0, X⁷ is O or N, and when q is 1, X⁷ is CR¹⁰.

Also disclosed herein is a compound according to the invention having the structural formula (2a):

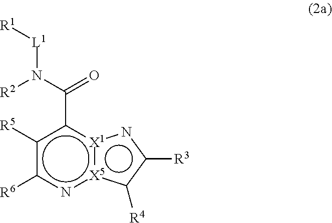

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein X¹, X⁵, L¹, R¹, R², R³, R₄, R⁵ and R⁶ are as defined for compounds of formula (1);
wherein one of X¹ and X⁵ is C and the other is N.

Also disclosed herein is a compound according to the invention having the structural formula (2aa):

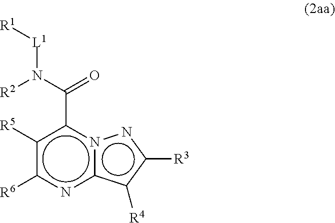

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein L¹, R¹, R², R³, R⁴, R⁵ and R⁶ are as defined for compounds of formula (1).

Also disclosed herein is a compound according to the invention having the structural formula (2ab):

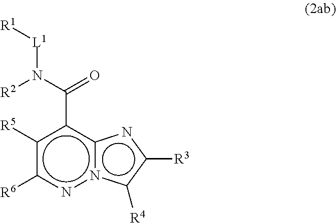

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein L¹, R¹, R², R³, R⁴, R⁵ and R⁶ are as defined for compounds of formula (1).

Also disclosed herein is a compound according to the invention having the structural formula (2ac):

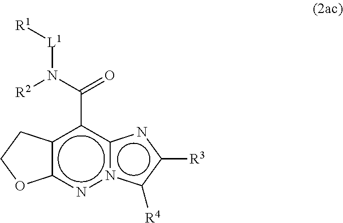

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein L¹, R¹, R², R³ and R⁴ are as defined for compounds of formula (1).

Also disclosed herein is a compound according to the invention having the structural formula (2ad):

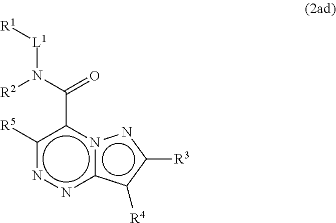

Including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein L¹, R¹, R², R³, R⁴, and R⁵ are as defined for compounds of formula (1).

Also disclosed herein is a compound according to the invention having the structural formula (2b):

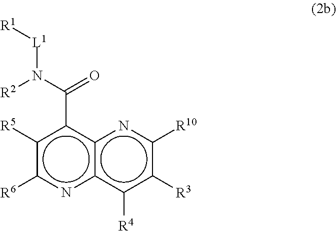

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein q, L¹, R¹, R², R³, R⁴, R⁵, R⁶ and R¹⁰ are as defined for compounds of formula (1).

Also disclosed herein is a compound according to the invention having the structural formula (3):

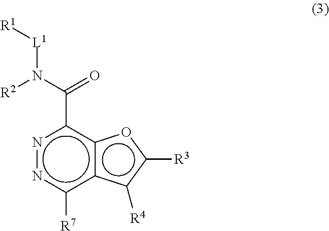

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein L¹, R¹, R², R³, R⁴ and R⁷ are as defined for compounds of formula (1).

Also disclosed herein is a compound according to the invention having the structural formula (4):

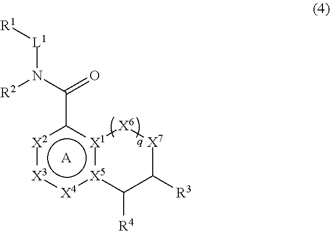

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein:
X¹, X², X³, X⁴, and X⁵ are as defined in clause 1;
X⁶ is selected from CR⁸R⁹ and O;
X⁷ is selected from CR¹¹R¹² and O; and
L¹, q, R¹, R², R³, R⁴, R⁸, R⁹, R¹¹, and R¹² are as defined for compounds of formula (1);
wherein when q is 0, X⁷ is O; and when X⁷ is O and q is 1, X⁶ is CR⁸R⁹.

Also disclosed herein is a compound according to the invention having the structural formula (4a):

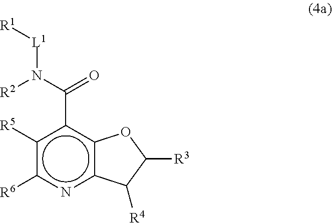

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein:
L¹, q, R¹, R², R³, R⁴, and R⁶ are as defined for compounds of formula (1).

Also disclosed herein is a compound according to the invention having the structural formula (4b)

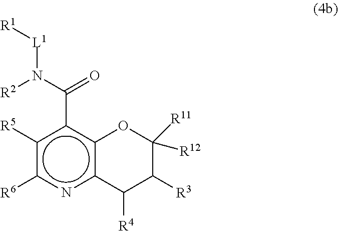

Including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein q, L¹, R¹, R², R³, R⁴, R⁵, R⁶, R¹¹ and R¹² are as defined for compounds of formula (1).

Also disclosed herein is a compound according to the invention having the structural formula (4c):

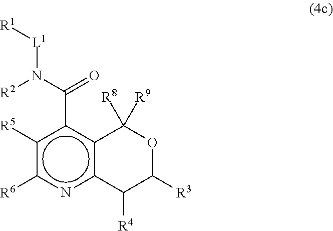

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein q, L¹, R¹, R², R³, R⁴, R⁵, R⁶, R⁸ and R⁹ are as defined for compounds of formula (1).

Also disclosed herein is a compound of formula (1) which has the structural formula (1a):

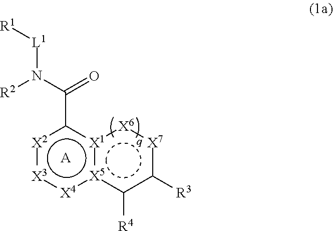

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein:
Q is O or S;

X¹ is selected from C and N;
X² is selected from CR⁵ and N;
X³ is selected from CR⁶ and N;
X⁴ is selected from CR⁷ and N;
X⁵ is selected from C and N;
wherein X¹, X², X³, X⁴ and X⁵ are selected so that the ring labelled A is aromatic;
X⁶ is selected from CR⁸R⁹, O and N;
X⁷ is selected from CR¹⁰, CR¹¹R¹², O and N;
q is 0 or 1;
wherein when q is 0, X⁷ is O or N; and when X⁷ is O or N and q is 1, X⁶ is CR₈R₉;
L¹ is a bond, C₁-C₆ alkylene, C₁-C₆ haloalkylene, C₃-C₆ cycloalkylene, or a 5- or 6-membered heterocycloalkyl having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more heteroatoms selected from N, O and S, 8- to 11-membered bicyclic heterocycloalkyl having one or more heteroatoms selected from N, O and S, phenyl, C₉-C₁₀ bicyclic aryl, 5- or 6-membered heteroaryl having one or more heteroatoms selected from N, O and S, 8- to 10-membered bicyclic heteroaryl having one or more heteroatoms selected from N, O and S, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S; R^{1a} and R^{1b} are each independently selected from H and C₁-C₄ alkyl;
R² is selected from H, C₁-C₄ alkyl and C₃-C₈ cycloakyl;
or R¹, R² and L¹ and the nitrogen atom to which R² and L¹ are attached form a 3- to 8-membered heterocycloalkyl having one or more heteroatoms selected from N, O and S, 8- to 11-membered bicyclic heterocycloalkyl having one or more heteroatoms selected from N, O and S, 5- or 6-membered heteroaryl having one or more heteroatoms selected from N, O and S, or 8- to 10-membered bicyclic heteroaryl having one or more heteroatoms selected from N, O and S, wherein said heterocycloalkyl, bicyclic heterocycloalkyl, heteroaryl and bicyclic heteroaryl are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy and C₁-C₄ alkyl-NR^{1a}R^{1b};
R³ is selected from H, OH, CN, NR^{3a}R^{3b}, C₁-C₄ alkyl, C₃-C₈ cycloalkyl, 3- to 8-membered heterocycloalkyl having one or more heteroatoms selected from N, O and S, phenyl, and 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, and C₃-C₈ cycloalkyl-C₁-C₄ alkoxy, wherein said alkyl, cycloalkyl, heterocycloalkyl, phenyl and heteroaryl are each optionally substituted by one or more substituents independently selected from halogen, OH, CN, C₁-C₄ alkyl, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl; R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} and R^{4b} are each independently selected from H and C₁-C₄ alkyl;
R⁵ and R⁶ are each independently selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S; or
R⁵ and R⁶ together with the carbon atoms to which they are attached form a 5- or 6-membered heterocycloalkyl having one or more heteroatoms selected from N, O and S; and
R⁷, R⁸, R⁹, R¹⁰, R¹¹, and R¹² are each independently selected from H, halogen, OH, CN, C₁-C₄ alkyl, C₁-C₄ haloalkyl, C₁-C₄ alkoxy, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S.

In embodiments of the compound of formula (1a),
L¹ is a bond, C₁-C₆ alkylene, C₁-C₆ haloalkylene, C₃-C₆ cycloalkylene, or a 5- or 6-membered heterocycloalkyl having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more heteroatoms selected from N, O and S, 8- to 11-membered bicyclic heterocycloalkyl having one or more heteroatoms selected from N, O and S, phenyl, C₉-C₁₀ bicyclic aryl, 5- or 6-membered heteroaryl having one or more heteroatoms selected from N, O and S, 8- to 10-membered bicyclic heteroaryl having one or more heteroatoms selected from N, O and S, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S; R^{1a} and R^{1b} are each independently selected from H and C₁-C₄ alkyl;
R² is selected from H, C₁-C₄ alkyl and C₃-C₈ cycloakyl;
R³ is selected from H, OH, CN, NR^{3a}R^{3b}, C₁-C₄ alkyl, C₃-C₈ cycloalkyl, 3- to 8-membered heterocycloalkyl having one or more heteroatoms selected from N, O and S, phenyl, and 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, and C₃-C₈ cycloalkyl-C₁-C₄ alkoxy, wherein said alkyl, cycloalkyl, heterocycloalkyl, phenyl and heteroaryl are each optionally substituted by one or more substituents independently selected from halogen, OH, CN, C₁-C₄ alkyl, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl; R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} and R^{4b} are each independently selected from H and C₁-C₄ alkyl;
R⁵ and R⁶ are each independently selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S; or
R⁵ and R⁶ together with the carbon atoms to which they are attached form a 5- or 6-membered heterocycloalkyl having one or more heteroatoms selected from N, O and S; and
R⁷, R⁸, R⁹, R¹⁰, R¹¹, and R¹² are each independently selected from H, halogen, OH, CN, C₁-C₄ alkyl, C₁-C₄ haloalkyl, C₁-C₄ alkoxy, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S.

In embodiments of the compound of formula (1a),
L¹ is a bond, C₁-C₆ alkylene, C₁-C₆ haloalkylene, C₃-C₆ cycloalkylene, or a 5- or 6-membered heterocycloalkyl having one or more heteroatoms selected from N, O and S;
R¹ is selected from C₁-C₄ alkyl, C₃-C₈ cycloalkyl, C₇-C₁₁ bicyclic cycloalkyl, 8- to 11-membered bicyclic heterocycloalkyl having one or more heteroatoms selected from N, O and S, phenyl, 5- or 6-membered heteroaryl having one or more heteroatoms selected from N, O and S, 8- to 10-membered bicyclic heteroaryl having one or more heteroatoms selected from N, O and S, and phenoxy, wherein said alkyl, cycloalkyl, bicyclic cycloalkyl, bicyclic heterocycloalkyl, phenyl, heteroaryl, bicyclic heteroaryl, and phenoxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, 5- or 6-membered heteroaryl having one or more heteroatoms selected from N, O and S, benzyl, halophenoxy, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, and C₃-C₈ cycloalkyl-C₁-C₄ alkoxy, wherein said alkyl, cycloalkyl, heterocycloalkyl, phenyl and heteroaryl are each optionally substituted by one or more substituents independently selected from halogen, OH, C₁-C₄ alkyl, and NR^{3c}R^{3d};
R^{3a} is selected from H, C₁-C₄ alkyl, and C₁-C₄ alkyl-NR^{3e}R^{3f};
R^{3b} is selected from H and C₁-C₄ alkyl;
R^{3c} is selected from H and C₁-C₄ alkyl;
R^{3d} is selected from C₁-C₄ alkyl;
R^{3e} and R^{3f} are each independently selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments of the compound of formula (1a),
L¹ is a bond, C₁-C₆ alkylene, C₁-C₆ haloalkylene, or C₃-C₆ cycloalkylene;
R¹ is selected from C₁-C₄ alkyl, C₃-C₈ cycloalkyl, C₇-C₁₁ bicyclic cycloalkyl, 8- to 11-membered bicyclic heterocycloalkyl having one or more heteroatoms selected from N, O and S, phenyl, 5- or 6-membered heteroaryl having one or more heteroatoms selected from N, O and S, 8- to 10-membered bicyclic heteroaryl having one or more heteroatoms selected from N, O and S, and phenoxy, wherein said alkyl, cycloalkyl, bicyclic cycloalkyl, bicyclic heterocycloalkyl, phenyl, heteroaryl, bicyclic heteroaryl, and phenoxy are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 5- or 6-membered heteroaryl having one or more heteroatoms selected from N, O and S, benzyl, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, and C₃-C₈ cycloalkyl-C₁-C₄ alkoxy, wherein said alkyl, cycloalkyl, heterocycloalkyl, phenyl and heteroaryl are each optionally substituted by one or more substituents independently selected from halogen, OH and NR^{3c}R^{3d}; R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments of the compound of formula (1a),
L¹ is a bond, C₁-C₆ alkylene, C₁-C₆ haloalkylene, or C₃-C₆ cycloalkylene;
R¹ is selected from C₃-C₈ cycloalkyl, C₇-C₁₁ bicyclic cycloalkyl, 8- to 11-membered bicyclic heterocycloalkyl having one or more heteroatoms selected from N, O and S, phenyl, 5- or 6-membered heteroaryl having one or more heteroatoms selected from N, O and S, and 8- to 10-membered bicyclic heteroaryl having one or more heteroatoms selected from N, O and S, wherein said cycloalkyl, bicyclic cycloalkyl, bicyclic heterocycloalkyl, phenyl, heteroaryl, and bicyclic heteroaryl are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, and 5- or 6-membered heteroaryl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, and C₃-C₈ cycloalkyl-C₁-C₄ alkoxy, wherein said alkyl, cycloalkyl, heterocycloalkyl, phenyl and heteroaryl are each optionally substituted by one or more substituents independently selected from halogen, OH and NR^{3c}R^{3d};
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from the following groups:

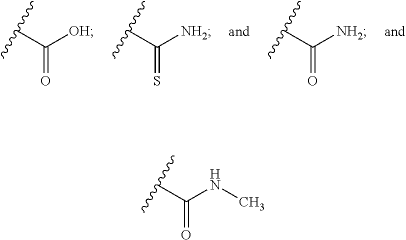

wherein [symbol] indicates the point of attachment to the rest of the compound;
R⁵ is selected from H and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments of the compound of formula (1a),
L¹ is a bond, C₁-C₆ alkylene, or C₁-C₆ haloalkylene;
R¹ is selected from C₃-C₈ cycloalkyl, C₇-C₁₁ bicyclic cycloalkyl, 8- to 11-membered bicyclic heterocycloalkyl having one or more heteroatoms selected from N, O and S, phenyl, 5- or 6-membered heteroaryl having one or more heteroatoms selected from N, O and S, and 8- to 10-membered bicyclic heteroaryl having one or more heteroatoms selected from N, O and S, wherein said cycloalkyl, bicyclic cycloalkyl, bicyclic heterocycloalkyl, phenyl, heteroaryl, and bicyclic heteroaryl are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, and 5- or 6-membered heteroaryl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, 3- to 8-membered heterocycloalkyl having one or more heteroatoms selected from N, O and S, phenyl, 5- or 6-membered heteroaryl having one or more heteroatoms selected from N, O and S, and C₁-C₄ alkoxy-C₁-C₄ alkyl, wherein said alkyl, heterocycloalkyl, phenyl and heteroaryl are each optionally substituted by one or more substituents independently selected from halogen and NR^{3c}R^{3d};
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is C(O)NR^{4a}R^{4b};
R^{4a} and R^{4b} are each H;
R⁵ is selected from H and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

In embodiments of the compound of formula (1a),
L¹ is either a bond, C₁-C₆ alkylene, or C₃-C₆ cycloalkylene;
R¹ is selected from C₃-C₈ cycloalkyl, C₇-C₁₁ bicyclic cycloalkyl, 8- to 11-membered bicyclic heterocycloalkyl having one or more heteroatoms selected from N, O and S, phenyl, 5- or 6-membered heteroaryl having one or more heteroatoms selected from N, O and S, and 8- to 10-membered bicyclic heteroaryl having one or more heteroatoms selected from N, O and S, wherein said cycloalkyl, bicyclic cycloalkyl, bicyclic heterocycloalkyl, phenyl, heteroaryl, and bicyclic heteroaryl are each optionally substituted by one or more substituents independently selected from halogen, C₁-C₄ alkyl, C₁-C₄ haloalkyl, C₁-C₄ alkoxy, and C₁-C₄ haloalkoxy;
R² is H;
R³ is selected from H, CN, NR^{3a}R^{3b}, C₁-C₄ alkyl optionally substituted by OH, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₃-C₈ cycloalkyl-C₁-C₄ alkoxy; and C₁-C₄ haloalkoxy-C₁-C₄ alkyl;
R^{3a} is H;
R^{3b} is selected from C₁-C₄ alkyl;

R⁴ is selected from the following groups:

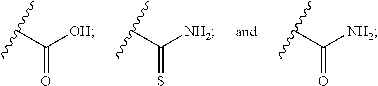

wherein [symbol] indicates the point of attachment to the rest of the compound;
R⁵ is H;
R⁶ is selected from H and C₁-C₄ alkyl; and
R⁷, R⁸, R⁹, R¹⁰, R¹¹, and R¹² are each H.

In embodiments of the compound of formula (1a),
L¹ is either a bond or C₁-C₆ alkylene;
R¹ is selected from C₃-C₈ cycloalkyl, C₈-C₁₁ bicyclic cycloalkyl, 8- to 11-membered bicyclic heterocycloalkyl having one or more heteroatoms selected from N, O and S, phenyl, 5- or 6-membered heteroaryl having one or more heteroatoms selected from N, O and S, and 8- to 10-membered bicyclic heteroaryl having one or more heteroatoms selected from N, O and S, wherein said cycloalkyl, bicyclic cycloalkyl, bicyclic heterocycloalkyl, phenyl, heteroaryl, and bicyclic heteroaryl are each optionally substituted by one or more substituents independently selected from halogen, C₁-C₄ alkyl, C₁-C₄ haloalkyl, C₁-C₄ alkoxy, and C₁-C₄ haloalkoxy;
R² is H;
R³ is selected from H, CN, NR^{3a}R^{3b}, C₁-C₄ alkyl, 3- to 8-membered heterocycloalkyl having one or more heteroatoms selected from N, O and S, 5- or 6-membered heteroaryl having one or more heteroatoms selected from N, O and S, and C₁-C₄ alkoxy-C₁-C₄ alkyl;
R^{3a} is H;
R^{3b} is selected from C₁-C₄ alkyl;
R⁴ is C(O)NR^{4a}R^{4b};
R^{4a} and R^{4b} are each H;
R⁵ is H;
R⁶ is selected from H and C₁-C₄ alkyl; and
R⁷, R⁸, R⁹, R¹⁰, R¹¹, and R¹² are each H.

In embodiments of the compound of formula (1a),
L¹ is either a bond or C₁-C₆ alkylene;
R¹ is selected from C₃-C₈ cycloalkyl, C₈-C₁₁ bicyclic cycloalkyl, 8- to 11-membered bicyclic heterocycloalkyl having one or more heteroatoms selected from N, O and S, phenyl, 5- or 6-membered heteroaryl having one or more heteroatoms selected from N, O and S, and 8- to 10-membered bicyclic heteroaryl having one or more heteroatoms selected from N, O and S, wherein said cycloalkyl, bicyclic cycloalkyl, bicyclic heterocycloalkyl, phenyl, heteroaryl, and bicyclic heteroaryl are each optionally substituted by one or more substituents independently selected from halogen, C₁-C₄ alkyl, C₁-C₄ haloalkyl, C₁-C₄ alkoxy, and C₁-C₄ haloalkoxy;
R² is H;
R³ is selected from NR^{3a}R^{3b}, C₁-C₄ alkyl substituted by one or more substituents independently selected from OH and NR^{3c}R^{3d}, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, and C₁-C₄ haloalkoxy-C₁-C₄ alkyl;
R^{3a} is H;
R^{3b} is selected from C₁-C₄ alkyl;
R⁴ is selected from the following groups:

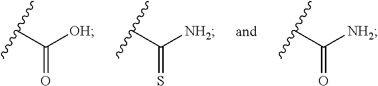

wherein [symbol] indicates the point of attachment to the rest of the compound;
R⁵, R⁶, R⁷, R⁸, R⁹, R¹⁰, R¹¹, and R¹² are each H.

In embodiments of the compound of formula (1a),
L¹ is either a bond or C₁-C₆ alkylene;
R¹ is selected from C₃-C₈ cycloalkyl, C₈-C₁₁ bicyclic cycloalkyl, 8- to 11-membered bicyclic heterocycloalkyl having one or more heteroatoms selected from N, O and S, phenyl, and 5- or 6-membered heteroaryl having one or more heteroatoms selected from N, O and S, wherein said cycloalkyl, bicyclic cycloalkyl, bicyclic heterocycloalkyl, phenyl, and heteroaryl are each optionally substituted by one or more substituents independently selected from halogen, C₁-C₄ alkyl, C₁-C₄ haloalkyl, C₁-C₄ alkoxy, and C₁-C₄ haloalkoxy;
R² is H;
R³ is selected from NR^{3a}R^{3b}, C₁-C₄ alkyl, 3- to 8-membered heterocycloalkyl having one or more heteroatoms selected from N, O and S, 5- or 6-membered heteroaryl having one or more heteroatoms selected from N, O and S, and C₁-C₄ alkoxy-C₁-C₄ alkyl;
R^{3a} is H;
R^{3b} is selected from C₁-C₄ alkyl;
R⁴ is C(O)NR^{4a}R^{4b};
R^{4a} and R^{4b} are each H;
R⁵, R⁶, R⁷, R⁸, R⁹, R¹⁰, R¹¹, and R¹² are each H.

In embodiments of the compound of formula (1a), Q is O.

In embodiments of the compound of formula (1a), L¹ is either a bond or C₁-C₆ alkylene (e.g. methylene).

In embodiments of the compound of formula (1a),
R¹ is selected from:
   C₃-C₈ cycloalkyl optionally substituted by one or more substituents independently selected from halogen and C₁-C₄ haloalkoxy;
   C₈-C₁₁ bicyclic cycloalkyl optionally substituted by one or more substituents independently selected from halogen and C₁-C₄ alkoxy;
   8- to 11-membered bicyclic heterocycloalkyl having one or more heteroatoms selected from N, O and S, optionally substituted by one or more substituents independently selected from halogen;
   phenyl optionally substituted by one or more substituents independently selected from halogen and C₁-C₄ alkyl;
   5- or 6-membered heteroaryl having one or more heteroatoms selected from N, O and S, optionally substituted by one or more substituents independently selected from C₁-C₄ alkyl and C₁-C₄ haloalkyl; and
   8- to 10-membered bicyclic heteroaryl having one or more heteroatoms selected from N, O and S, optionally substituted substituted by one or more substituents independently selected from halogen and C₁-C₄ alkyl.

In embodiments of the compound of formula (1a),
R² is H.

In embodiments of the compound of formula (1a),
R³ is selected from:
   NR^{3a}R^{3b};
   C₁-C₄ alkyl optionally substituted by OH;
   C₃-C₈ cycloalkyl;
   3- to 8-membered heterocycloalkyl having one or more heteroatoms selected from N, O and S;
   5- or 6-membered heteroaryl having one or more heteroatoms selected from N, O and S;
   C₁-C₄ alkoxy-C₁-C₄ alkyl;
   C₃-C₈ cycloalkyl-C₁-C₄ alkoxy; and
   C₁-C₄ haloalkoxy-C₁-C₄ alkyl;
wherein
R^{3a} is H; and
R^{3b} is selected from C₁-C₄ alkyl.

In embodiments of the compound of formula (1a), R³ is selected from C₁-C₄ alkoxy-C₁-C₄ alkyl.

In embodiments of the compound of formula (1a),
R⁴ is selected from the following groups:

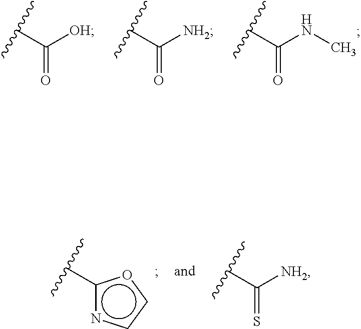

wherein [symbol] indicates the point of attachment to the rest of the compound.

In embodiments of the compound of formula (1a),
R⁴ is selected from C(O)OH, C(O)NR^{4a}R^{4b} and C(S)NR^{4a}R^{4b}; and
R^{4a} and R^{4b} are each H.

In embodiments of the compound of formula (1a),
R⁵ is selected from the following groups:

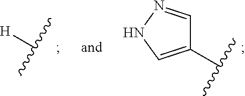

wherein [symbol] indicates the point of attachment to the rest of the compound.

In embodiments of the compound of formula (1a), R⁵ is H.

In embodiments of the compound of formula (1a), R⁶ is H or methyl.

In embodiments of the compound of formula (1a), R⁶ is H.

In embodiments of the compound of formula (1a), R⁷, R⁸, R⁹, R¹⁰, R¹¹, and R¹² are each H.

Also disclosed herein is a compound according to the invention having the structural formula (2′a):

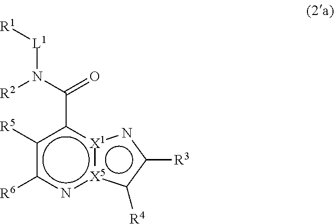

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein X¹, X⁵, L¹, R¹, R², R³, R₄, R⁵ and R⁶ are as defined for compounds of formula (1a);
wherein one of X¹ and X⁵ is C and the other is N.

Also disclosed herein is a compound of formula (1a) having the structural formula (2′aa):

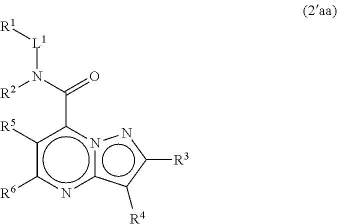

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein L¹, R¹, R², R³, R⁴, R⁵ and R⁶ are as defined for compounds of formula (1a).

Also disclosed herein is a compound of formula (1a) having the structural formula (2′ab):

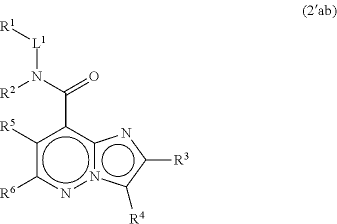

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein L¹, R¹, R², R³, R⁴, R⁵ and R⁶ are as defined for compounds of formula (1a).

Also disclosed herein is a compound of formula (1a) having the structural formula (2′ad):

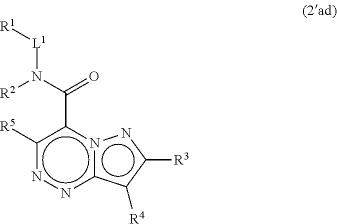

Including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein L¹, R¹, R², R³, R⁴, and R⁵ are as defined for compounds of formula (1a).

Also disclosed herein is a compound of formula (1) which has the structural formula (1b):

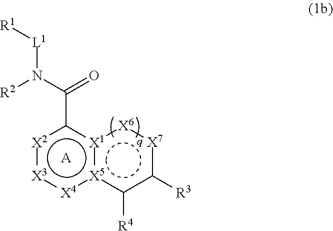

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein:
X¹ is selected from C and N;
X² is selected from CR⁵ and N;
X³ is selected from CR⁶ and N;
X⁴ is selected from CR⁷ and N;
X⁵ is selected from C and N;
wherein X¹, X², X³, X⁴ and X⁵ are selected so that the ring labelled A is aromatic;
X⁶ is selected from CR⁸R⁹, O and N;
X⁷ is selected from CR¹⁰, CR¹¹R¹², O and N;
q is 0 or 1;
wherein when q is 0, X⁷ is O or N; and when X⁷ is O or N and q is 1, X⁶ is CR⁸R⁹;
L¹ is a bond, C₁-C₆ alkylene or a 5- or 6-membered heterocycloalkyl having one or more heteroatoms selected from N, O and S;
R¹ is selected from C₁-C₄ alkyl, C₃-C₈ cycloalkyl, C₈-C₁₁ bicyclic cycloalkyl, 3- to 8-membered heterocycloalkyl having one or more heteroatoms selected from N, O and S, 8- to 11-membered bicyclic heterocycloalkyl having one or more heteroatoms selected from N, O and S, phenyl, C₉-C₁₀ bicyclic aryl, 5- or 6-membered heteroaryl having one or more heteroatoms selected from N, O and S, 8- to 10-membered bicyclic heteroaryl having one or more heteroatoms selected from N, O and S, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄haloalkyl, C₁-C₄alkoxy, C₁-C₄haloalkoxy, C₁-C₄alkyl-NR^{1a}R^{1b}, C₃-C₈cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy and 5- or 6-membered heteroaryloxy having one or more heteroatoms selected from N, O and S;
R^{1a} and R^{1b} are each independently selected from H and C₁-C₄alkyl;
R² is selected from H, C₁-C₄ alkyl and C₃-C₈ cycloakyl;
or R¹, R² and L¹ and the nitrogen atom to which R² and L¹ are attached form a 3- to 8-membered heterocycloalkyl having one or more heteroatoms selected from N, O and S, 8- to 11-membered bicyclic heterocycloalkyl having one or more heteroatoms selected from N, O and S, 5- or 6-membered heteroaryl having one or more heteroatoms selected from N, O and S, or 8- to 10-membered bicyclic heteroaryl having one or more heteroatoms selected from N, O and S, wherein said heterocycloalkyl, bicyclic heterocycloalkyl, heteroaryl and bicyclic heteroaryl are each optionally substituted by one or more substituents independently selected from halogen, OH, CN, NR^{1a}R^{1b}, C₁-C₄ alkyl, C₁-C₄haloalkyl, C₁-C₄alkoxy, C₁-C₄haloalkoxy and C₁-C₄alkyl-NR^{1a}R^{1b};
R³ is selected from H, OH, CN, NR^{3a}R^{3b}, C₁-C₄ alkyl, C₃-C₈ cycloalkyl, 3- to 8-membered heterocycloalkyl having one or more heteroatoms selected from N, O and S, phenyl, and 5- or 6-membered heteroaryl having one or more heteroatoms selected from N, O and S, C₁-C₄alkoxy-C₁-C₄alkyl, C₁-C₄haloalkoxy-C₁-C₄alkyl and C₃-C₈cycloalkoxy-C₁-C₄alkyl, wherein said alkyl, cycloalkyl, heterocycloalkyl, phenyl and heteroaryl are each optionally substituted by one or more substituents independently selected from halogen, OH, CN, C₁-C₄ alkyl, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl;
R⁴ is selected from C(O)NR^{4a}R^{4b} and 5- or 6-membered heteroaryl having one or more heteroatoms selected from N, O and S;
R^{4a} and R^{4b} are each independently selected from H and C₁-C₄ alkyl;
R⁵ and R⁶ are each independently selected from H, halogen, OH, CN, C₁-C₄ alkyl, C₁-C₄ haloalkyl, C₁-C₄ alkoxy, C₁-C₄alkoxy-C₁-C₄alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S; or
R⁵ and R⁶ together with the carbon atoms to which they are attached form a 5- or 6-membered heterocycloalkyl having one or more heteroatoms selected from N, O and S; and
R⁷, R⁸, R⁹, R¹⁰, R¹¹, and R¹² are each independently selected from H, halogen, OH, CN, C₁-C₄ alkyl, C₁-C₄ haloalkyl, C₁-C₄ alkoxy, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S.

In embodiments of the compound of formula (1b),
L¹ is a bond, C₁-C₆ alkylene or a 5- or 6-membered heterocycloalkyl having one or more heteroatoms selected from N, O and S;
R¹ is selected from C₁-C₄ alkyl, C₃-C₈ cycloalkyl, C₈-C₁₁ bicyclic cycloalkyl, 3- to 8-membered heterocycloalkyl having one or more heteroatoms selected from N, O and S, 8- to 11-membered bicyclic heterocycloalkyl having one or more heteroatoms selected from N, O and S, phenyl, C₉-C₁₀ bicyclic aryl, 5- or 6-membered heteroaryl having one or more heteroatoms selected from N, O and S, 8- to 10-membered bicyclic heteroaryl having one or more heteroatoms selected from N, O and S, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄haloalkyl, C₁-C₄alkoxy, C₁-C₄haloalkoxy, C₁-C₄alkyl-NR^{1a}R^{1b}, C₃-C₈cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy and 5- or 6-membered heteroaryloxy having one or more heteroatoms selected from N, O and S;
R^{1a} and R^{1b} are each independently selected from H and C₁-C₄alkyl;
R² is selected from H, C₁-C₄ alkyl and C₃-C₈ cycloakyl;
R³ is selected from H, OH, CN, NR^{3a}R^{3b}, C₁-C₄ alkyl, C₃-C₈ cycloalkyl, 3- to 8-membered heterocycloalkyl having one or more heteroatoms selected from N, O and S, phenyl, and 5- or 6-membered heteroaryl having one or more heteroatoms selected from N, O and S, C₁-C₄alkoxy-C₁-C₄alkyl, C₁-C₄haloalkoxy-C₁-C₄alkyl and C₃-C₈cycloalkoxy-C₁-C₄alkyl, wherein said alkyl, cycloalkyl, heterocycloalkyl, phenyl and heteroaryl are each optionally substituted by one or more substituents independently selected from halogen, OH, CN, C₁-C₄ alkyl, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl;
R⁴ is selected from C(O)NR^{4a}R^{4b} and 5- or 6-membered heteroaryl having one or more heteroatoms selected from N, O and S;
R^{4a} and R^{4b} are each independently selected from H and C₁-C₄ alkyl;
R⁵ and R⁶ are each independently selected from H, halogen, OH, CN, C₁-C₄ alkyl, C₁-C₄ haloalkyl, C₁-C₄ alkoxy, C₁-C₄alkoxy-C₁-C₄alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S; or
R⁵ and R⁶ together with the carbon atoms to which they are attached form a 5- or 6-membered heterocycloalkyl having one or more heteroatoms selected from N, O and S; and
R⁷, R⁸, R⁹, R¹⁰, R¹¹, and R¹² are each independently selected from H, halogen, OH, CN, C₁-C₄ alkyl, C₁-C₄ haloalkyl, C₁-C₄ alkoxy, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S.

In embodiments of the compound of formula (1b),
L¹ is a bond, C₁-C₆ alkylene or a 5- or 6-membered heterocycloalkyl having one or more heteroatoms selected from N, O and S;
R¹ is selected from C₁-C₄ alkyl, C₃-C₈ cycloalkyl, C₈-C₁₁ bicyclic cycloalkyl, 8- to 11-membered bicyclic heterocycloalkyl having one or more heteroatoms selected from N, O and S, phenyl, 5- or 6-membered heteroaryl having one or more heteroatoms selected from N, O and S, 8- to 10-membered bicyclic heteroaryl having one or more heteroatoms selected from N, O and S, and phenoxy, wherein said alkyl, cycloalkyl, bicyclic cycloalkyl, bicyclic heterocycloalkyl, phenyl, heteroaryl, bicyclic heteroaryl, and phenoxy are each optionally substituted by one or more substituents independently selected from halogen, OH, CN, NR^{1a}R^{1b}, C₁-C₄ alkyl, C₁-C₄haloalkyl, C₁-C₄alkoxy, C₁-C₄haloalkoxy, C₁-C₄alkyl-NR^{1a}R^{1b}, C₃-C₈cycloalkyl, 3- to 8-membered heterocycloalkyl having one or more heteroatoms selected from N, O and S, 5- or 6-membered heteroaryl having one or more heteroatoms selected from N, O and S, benzyl, and halophenoxy;
R^{1a} and R^{1b} are each independently selected from H and C₁-C₄alkyl;
R² is H;
R³ is selected from H, CN, NR^{3a}R^{3b}, C₁-C₄ alkyl, 3- to 8-membered heterocycloalkyl having one or more heteroatoms selected from N, O and S, phenyl, 5- or 6-membered heteroaryl having one or more heteroatoms selected from N, O and S, and C₁-C₄alkoxy-C₁-C₄alkyl, wherein said alkyl, heterocycloalkyl, phenyl and heteroaryl are each optionally substituted by one or more substituents independently selected from halogen, C₁-C₄ alkyl, and NR^{3c}R^{3d};
R^{3a} is selected from H, C₁-C₄ alkyl, and C₁-C₄ alkyl-NR^{3e}R^{3f};
R^{3b} is selected from H and C₁-C₄ alkyl;
R^{3c} is selected from H and C₁-C₄ alkyl;
R^{3d} is selected from C₁-C₄ alkyl;
R^{3e} and R^{3f} are each independently selected from C₁-C₄ alkyl;
R⁴ is selected from C(O)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄alkoxy-C₁-C₄alkyl; and
R⁷, R⁸, R⁹, R¹⁰, R¹¹, and R¹² are each H.

In embodiments of the compound of formula (1b),
L¹ is either a bond or C₁-C₆ alkylene;
R¹ is selected from C₁-C₄ alkyl, C₃-C₈ cycloalkyl, C₈-C₁₁ bicyclic cycloalkyl, 8- to 11-membered bicyclic heterocycloalkyl having one or more heteroatoms selected from N, O and S, phenyl, 5- or 6-membered heteroaryl having one or more heteroatoms selected from N, O and S, 8- to 10-membered bicyclic heteroaryl having one or more heteroatoms selected from N, O and S, and phenoxy, wherein said alkyl, cycloalkyl, bicyclic cycloalkyl, bicyclic heterocycloalkyl, phenyl, heteroaryl, bicyclic heteroaryl, and phenoxy are each optionally substituted by one or more substituents independently selected from halogen, CN, OH, C₁-C₄ alkyl, C₁-C₄haloalkyl, C₁-C₄alkoxy, C₁-C₄haloalkoxy, C₁-C₄alkyl-NR^{1a}R^{1b}, C₃-C₈cycloalkyl, 5- or 6-membered heteroaryl having one or more heteroatoms selected from N, O and S, and benzyl;
R^{1a} and R^{1b} are each independently selected from H and C₁-C₄alkyl;
R² is H;
R³ is selected from H, CN, NR^{3a}R^{3b}, C₁-C₄ alkyl, 3- to 8-membered heterocycloalkyl having one or more heteroatoms selected from N, O and S, phenyl, 5- or 6-membered heteroaryl having one or more heteroatoms selected from N, O and S, and C₁-C₄alkoxy-C₁-C₄alkyl, wherein said alkyl, heterocycloalkyl, phenyl and heteroaryl are each optionally substituted by one or more substituents independently selected from halogen and NR^{3c}R^{3d};
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from C(O)NR^{4a}R^{4b} and 5- or 6-membered heteroaryl having one or more heteroatoms selected from N, O and S;
R^{4a} and R^{4b} are each H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄alkoxy-C₁-C₄alkyl; and
R⁷, R⁸, R⁹, R¹⁰, R¹¹, and R¹² are each H.

In embodiments of the compound of formula (1b),
L¹ is either a bond or C₁-C₆ alkylene;
R¹ is selected from C₃-C₈ cycloalkyl, C₈-C₁₁ bicyclic cycloalkyl, 8- to 11-membered bicyclic heterocycloalkyl having one or more heteroatoms selected from N, O and S, phenyl, 5- or 6-membered heteroaryl having one or more heteroatoms selected from N, O and S, and 8- to 10-membered bicyclic heteroaryl having one or more heteroatoms selected from N, O and S, wherein said cycloalkyl, bicyclic cycloalkyl, bicyclic heterocycloalkyl, phenyl, heteroaryl, and bicyclic heteroaryl are each optionally substituted by one or more substituents independently selected from halogen, CN, OH, C₁-C₄ alkyl, C₁-C₄haloalkyl, C₁-C₄alkoxy, C₁-C₄haloalkoxy, C₁-C₄alkyl-NR^{1a}R^{1b}, and 5- or 6-membered heteroaryl having one or more heteroatoms selected from N, O and S;
R^{1a} and R^{1b} are each independently selected from H and C₁-C₄alkyl;
R² is H;
R³ is selected from H, CN, NR^{3a}R^{3b}, C₁-C₄ alkyl, 3- to 8-membered heterocycloalkyl having one or more heteroatoms selected from N, O and S, phenyl, 5- or 6-membered heteroaryl having one or more heteroatoms selected from N, O and S, and C₁-C₄alkoxy-C₁-C₄alkyl, wherein said alkyl, heterocycloalkyl, phenyl and heteroaryl are each optionally substituted by one or more substituents independently selected from halogen and NR^{3c}R^{3d};
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is C(O)NR^{4a}R^{4b};
R^{4a} and R^{4b} are each H;
R⁵ is selected from H and 5- or 6-membered heteroaryl having one or more heteroatoms selected from N, O and S;
R⁶ is selected from H, C₁-C₄ alkyl, C₁-C₄ alkoxy, and C₁-C₄alkoxy-C₁-C₄alkyl; and
R⁷, R⁸, R⁹, R¹⁰, R¹¹, and R¹² are each H.

In embodiments of the compound of formula (1b),
L¹ is either a bond or C₁-C₆ alkylene;
R¹ is selected from C₃-C₈ cycloalkyl, C₈-C₁₁ bicyclic cycloalkyl, 8- to 11-membered bicyclic heterocycloalkyl having one or more heteroatoms selected from N, O and S, phenyl, 5- or 6-membered heteroaryl having one or more heteroatoms selected from N, O and S, and 8- to 10-membered bicyclic heteroaryl having one or more heteroatoms selected from N, O and S, wherein said cycloalkyl, bicyclic cycloalkyl, bicyclic heterocycloalkyl, phenyl, heteroaryl, and bicyclic heteroaryl are each optionally substituted by one or more substituents independently selected from halogen, C₁-C₄ alkyl, C₁-C₄haloalkyl, C₁-C₄alkoxy, and C₁-C₄haloalkoxy;
R² is H;
R³ is selected from H, CN, NR^{3a}R^{3b}, C₁-C₄ alkyl, 3- to 8-membered heterocycloalkyl having one or more heteroatoms selected from N, O and S, 5- or 6-membered heteroaryl having one or more heteroatoms selected from N, O and S, and C₁-C₄alkoxy-C₁-C₄alkyl;
R^{3a} is H;
R^{3b} is selected from C₁-C₄ alkyl;
R⁴ is C(O)NR^{4a}R^{4b};
R^{4a} and R^{4b} are each H;
R⁵ is H;
R⁶ is selected from H and C₁-C₄ alkyl; and
R⁷, R⁸, R⁹, R¹⁰, R¹¹, and R¹² are each H.

In embodiments of the compound of formula (1b),
L¹ is either a bond or C₁-C₆ alkylene;
R¹ is selected from C₃-C₈ cycloalkyl, C₈-C₁₁ bicyclic cycloalkyl, 8- to 11-membered bicyclic heterocycloalkyl having one or more heteroatoms selected from N, O and S, phenyl, and 5- or 6-membered heteroaryl having one or more heteroatoms selected from N, O and S, wherein said cycloalkyl, bicyclic cycloalkyl, bicyclic heterocycloalkyl, phenyl, and heteroaryl are each optionally substituted by one or more substituents independently selected from halogen, C₁-C₄ alkyl, C₁-C₄haloalkyl, C₁-C₄alkoxy, and C₁-C₄haloalkoxy;
R² is H;
R³ is selected from NR^{3a}R^{3b}, C₁-C₄ alkyl, 3- to 8-membered heterocycloalkyl having one or more heteroatoms selected from N, O and S, 5- or 6-membered heteroaryl having one or more heteroatoms selected from N, O and S, and C₁-C₄alkoxy-C₁-C₄alkyl;
R^{3a} is H;
R^{3b} is selected from C₁-C₄ alkyl;
R⁴ is C(O)NR^{4a}R^{4b};
R^{4a} and R^{4b} are each H;
R⁵, R⁶, R⁷, R⁸, R⁹, R¹⁰, R¹¹, and R¹² are each H.

In embodiments of the compound of formula (1b), L¹ is either a bond or methylene.

In embodiments of the compound of formula (1b),
R¹ is selected from:
   C₃-C₈ cycloalkyl optionally substituted by one or more substituents independently selected from halogen and C₁-C₄haloalkoxy;
   C₈-C₁₁ bicyclic cycloalkyl optionally substituted by one or more substituents independently selected from halogen and C₁-C₄alkoxy;
   8- to 11-membered bicyclic heterocycloalkyl having one or more heteroatoms selected from N, O and S;
   phenyl optionally substituted by one or more substituents independently selected from halogen and C₁-C₄ alkyl; and
   5- or 6-membered heteroaryl having one or more heteroatoms selected from N, O and S, optionally substituted by one or more substituents independently selected from C₁-C₄ alkyl and C₁-C₄ haloalkyl.

In embodiments of the compound of formula (1b),
R² is H.

In embodiments of the compound of formula (1b),
R³ is selected from:
   NR^{3a}R^{3b};
   C₁-C₄ alkyl;
   3- to 8-membered heterocycloalkyl having one or more heteroatoms selected from N, O and S;
   5- or 6-membered heteroaryl having one or more heteroatoms selected from N, O and S; and
   C₁-C₄alkoxy-C₁-C₄alkyl;
wherein
R^{3a} is H; and
R^{3b} is selected from C₁-C₄ alkyl.

In embodiments of the compound of formula (1b),
R³ is selected from C₁-C₄alkoxy-C₁-C₄alkyl.

In embodiments of the compound of formula (1b),
R⁴ is selected from the following groups:

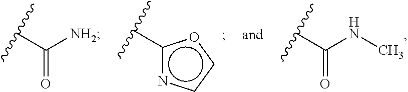

wherein [symbol] indicates the point of attachment to the rest of the compound.

In embodiments of the compound of formula (1b),
R⁵ is H.

In embodiments of the compound of formula (1b),
R⁶ is H.

In embodiments of the compound of formula (1b),
R⁷, R⁸, R⁹, R¹⁰, R¹¹, and R¹² are each H.

Also disclosed herein is a compound of formula (1b) having the structural formula (2″a):

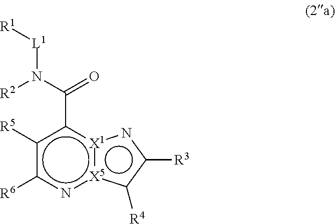

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein X¹, X⁵, L¹, R¹, R², R³, R⁴, R⁵ and R⁶ are as defined for compounds of formula (1b)
wherein one of X¹ and X⁵ is C and the other is N.

Also disclosed herein is a compound of formula (1b) having the structural formula (2″aa):

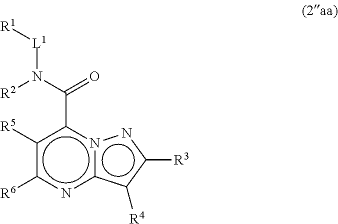

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein L¹, R¹, R², R³, R⁴, R⁵ and R⁶ are as defined for compounds of formula (1b).

Also disclosed herein is a compound of formula (1b) having the structural formula (2″ab):

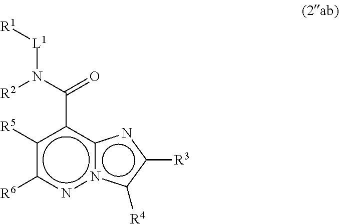

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein L¹, R¹, R², R³, R⁴, R⁵ and R⁶ are as defined for compounds of formula (1b).

Also disclosed herein is a compound according to the invention having the structural formula (2′):

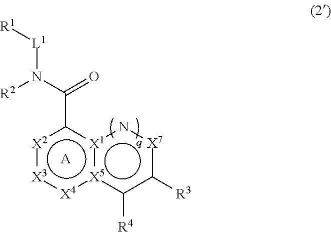

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein:
X⁷ is selected from CR¹⁰, O, and N; and
X¹, X², X³, X⁴, X⁵, q, L¹, R¹, R², R³, R⁴, and R¹⁰ are as defined for compounds of formula (1a);
wherein when q is 0, X⁷ is O or N, and when q is 1, X⁷ is CR¹⁰.

Also disclosed herein is a compound according to the invention having the structural formula (2′a):

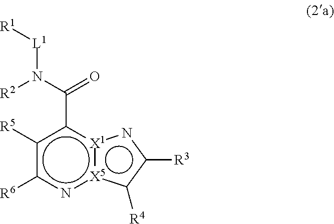

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein X¹, X⁵, L¹, R¹, R², R³, R⁴, R⁵ and R⁶ are as defined for compounds of formula (1a); and
wherein one of X¹ and X⁵ is C and the other is N.

Also disclosed herein is a compound according to the invention having the structural formula (2′aa):

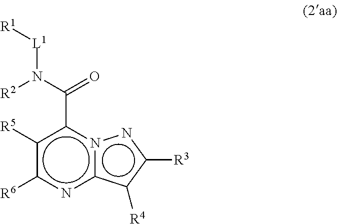

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein L¹, R¹, R², R³, R⁴, R⁵ and R⁶ are as defined for compounds of formula (1a).

Also disclosed herein is a compound according to the invention having the structural formula (2′ab):

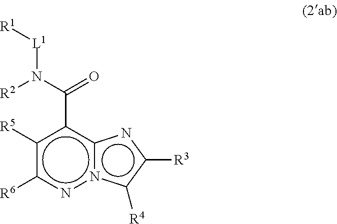

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein L¹, R¹, R², R³, R⁴, R⁵ and R⁶ are as defined for compounds of formula (1a).

Also disclosed herein is a compound according to the invention having the structural formula (2′ac):

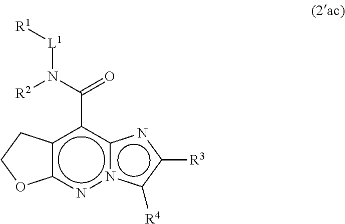

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein L¹, R¹, R², R³ and R⁴ are as defined for compounds of formula (1a).

Also disclosed herein is a compound according to the invention having the structural formula (2′b):

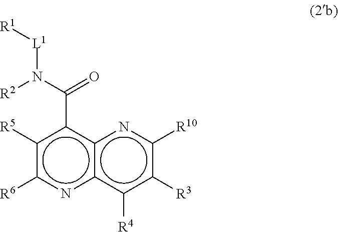

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein q, L¹, R¹, R², R³, R⁴, R⁵, R⁶ and R¹⁰ are as defined for compounds of formula (1a).

Also disclosed herein is a compound according to the invention having the structural formula (3′).

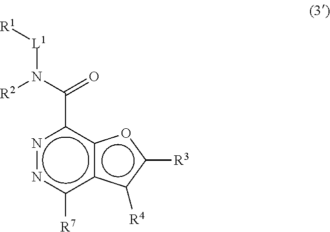

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein L¹, R¹, R², R³, R⁴ and R⁷ are as defined for compounds of formula (1a).

Also disclosed herein is a compound according to the invention having the structural formula (4′).

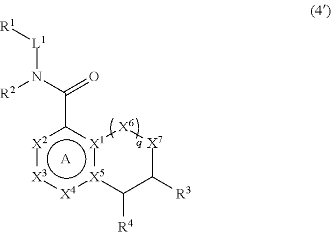

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein:
X⁶ is selected from CR⁸R⁹ and O;
X⁷ is selected from CR¹¹R¹² and O; and
X¹, X², X³, X⁴, X⁵, L¹, q, R¹, R², R³, R⁴, R⁸, R⁹, R¹¹, and R¹² are as defined in any for compounds of formula (1a); and
wherein when q is 0, X⁷ is O; and when X⁷ is O and q is 1, X⁶ is CR⁸R⁹.

Also disclosed herein is a compound according to the invention having the structural formula (4′a):

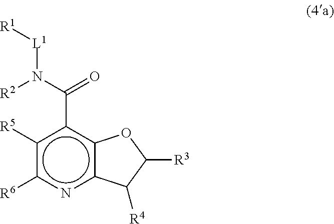

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein:
L¹, q, R¹, R², R³, R⁴, and R⁶ are as defined for compounds of formula (1a).

Also disclosed herein is a compound according to the invention having the structural formula

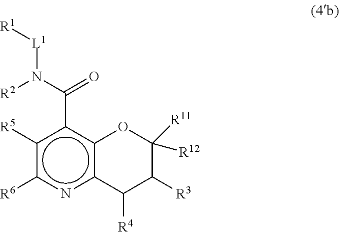

Including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein q, L¹, R¹, R², R³, R⁴, R⁵, R⁶, R¹¹ and R¹² are as defined for compounds of formula (1a).

Also disclosed herein is a compound according to the invention having the structural formula (4c):

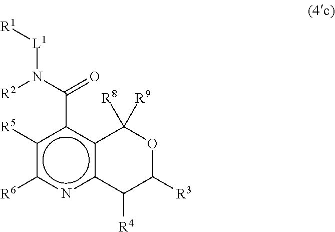

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein q, L¹, R¹, R², R³, R⁴, R⁵, R⁶, R⁸ and R⁹ are as defined for compounds of formula (1a).

Also disclosed herein is a compound according to the invention as listed in Table 1, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof. In embodiments, the compound is one of compounds 1 to 242, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof.

In embodiments, the compound may be selected from the compounds listed in Table 1.

TABLE 1
Exemplary compounds of the present disclosure

| Compound | |
|---|---|
| Number | Compound |
| 1 | N7-indan-2-ylpyrazolo[1,5-a]pyrimidine-3,7-dicarboxamide |
| 2 | N7-(4-methylindan-2-yl)pyrazolo[1,5-a]pyrimidine-3,7-dicarboxamide |
| 3 | N7-(5-methoxyindan-2-yl)pyrazolo[1,5-a]pyrimidine-3,7-dicarboxamide |
| 4 | N7-(4-chloroindan-2-yl)pyrazolo[1,5-a]pyrimidine-3,7-dicarboxamide |
| 5 | N7-(1,2,3,4-tetrahydroquinolin-3-yl)pyrazolo[1,5-a]pyrimidine-3,7- |
| | dicarboxamide |
| 6 | N7-chroman-3-ylpyrazolo[1,5-a]pyrimidine-3,7-dicarboxamide |
| 7 | N7-(trans-3-fluoro-3-methyl-cyclobutyl)pyrazolo[1,5-a]pyrimidine-3,7- |
| | dicarboxamide |
| 8 | N7-(cis-3-hydroxy-3-methyl-cyclobutyl)pyrazolo[1,5-a]pyrimidine-3,7- |
| | dicarboxamide |
| 9 | N7-(cis-3-cyanocyclobutyl)pyrazolo[1,5-a]pyrimidine-3,7-dicarboxamide |
| 10 | N7-(3,3-diethylcyclobutyl)pyrazolo[1,5-a]pyrimidine-3,7-dicarboxamide |
| 11 | N7-(cis-3-methoxycyclobutyl)pyrazolo[1,5-a]pyrimidine-3,7-dicarboxamide |
| 12 | N7-(7-oxaspiro[3.5]nonan-2-yl)pyrazolo[1,5-a]pyrimidine-3,7-dicarboxamide |
| 13 | N7-(5,6,7,8-tetrahydroisoquinolin-7-yl)pyrazolo[1,5-a]pyrimidine-3,7- |
| | dicarboxamide |
| 14 | N7-[4-(3,3,3-trifluoropropyl)indan-2-yl]pyrazolo[1,5-a]pyrimidine-3,7- |
| | dicarboxamide |
| 15 | N7-[5-(2-pyrrolidin-1-ylethyl)indan-2-yl]pyrazolo[1,5-a]pyrimidine-3,7- |
| | dicarboxamide |
| 16 | N7-[4-(4-chlorophenoxy)indan-2-yl]pyrazolo[1,5-a]pyrimidine-3,7- |
| | dicarboxamide |
| 17 | N7-[4-(2-pyridyl)indan-2-yl]pyrazolo[1,5-a]pyrimidine-3,7-dicarboxamide |
| 18 | N7-[cis-3-(trifluoromethoxy)cyclobutyl]pyrazolo[1,5-a]pyrimidine-3,7- |
| | dicarboxamide |
| 19 | N7-(6,7-dihydro-5H-pyrrolo[1,2-a]imidazol-6-yl)pyrazolo[1,5-a]pyrimidine- |
| | 3,7-dicarboxamide |
| 20 | N7-(1,2,3,4-tetrahydro-1,8-naphthyridin-3-yl)pyrazolo[1,5-a]pyrimidine-3,7- |
| | dicarboxamide |
| 21 | 2-cyano-N7-indan-2-yl-pyrazolo[1,5-a]pyrimidine-3,7-dicarboxamide |
| 22 | N7-indan-2-yl-2-pyridazin-3-yl-pyrazolo[1,5-a]pyrimidine-3,7-dicarboxamide |
| 23 | 2-[2-(dimethylamino)ethylamino]-N7-indan-2-yl-pyrazolo[1,5-a]pyrimidine- |
| | 3,7-dicarboxamide |
| 24 | N7-indan-2-yl-2-(1-methylazetidin-3-yl)pyrazolo[1,5-a]pyrimidine-3,7- |
| | dicarboxamide |
| 25 | N7-(3,3-difluorocyclobutyl)-2-oxazol-2-yl-pyrazolo[1,5-a]pyrimidine-3,7- |
| | dicarboxamide |
| 26 | N8-(3,3-difluorocyclobutyl)-2-methyl-imidazo[1,2-b]pyridazine-3,8- |
| | dicarboxamide |
| 27 | N7-indan-2-yl-2-(isopropoxymethyl)pyrazolo[1,5-a]pyrimidine-3,7- |
| | dicarboxamide |
| 28 | N7-(3,3-difluorocyclobutyl)-2-(isopropoxymethyl)pyrazolo[1,5-a]pyrimidine- |
| | 3,7-dicarboxamide |
| 29 | 2-[(dimethylamino)methyl]-N7-indan-2-yl-pyrazolo[1,5-a]pyrimidine-3,7- |
| | dicarboxamide |
| 30 | N7-indan-2-yl-2-(methylaminomethyl)pyrazolo[1,5-a]pyrimidine-3,7- |
| | dicarboxamide |
| 31 | N-indan-2-yl-3-oxazol-2-yl-pyrazolo[1,5-a]pyridine-7-carboxamide |
| 32 | N7-(3,3-difluorocyclobutyl)-2-methyl-6-(1 H-pyrazol-4-yl)pyrazolo[1,5- |
| | alpyrimidine-3,7-dicarboxamide |
| 33 | N7-indan-2-yl-2-oxazol-2-yl-pyrazolo[1,5-a]pyrimidine-3,7-dicarboxamide |
| 34 | N7-(3,3-difluorocyclobutyl)-2-(methylamino)pyrazolo[1,5-a]pyrimidine-3,7- |
| | dicarboxamide |
| 35 | N7-(3,3-difluorocyclobutyl)-2-(dimethylamino)pyrazolo[1,5-a]pyrimidine-3,7- |
| | dicarboxamide |
| 36 | 2-(2-fluorophenyl)-N7-indan-2-yl-pyrazolo[1,5-a]pyridine-3,7-dicarboxamide |
| 37 | N7-indan-2-yl-2-tetrahydrofuran-3-yl-pyrazolo[1,5-a]pyrimidine-3,7- |
| | dicarboxamide |
| 38 | N7-(3,3-difluorocyclobutyl)-2,6-dimethyl-pyrazolo[1,5-a]pyrimidine-3,7- |
| | dicarboxamide |
| 39 | N7-(3,3-difluorocyclobutyl)-5-(methoxymethyl)-2-methyl-pyrazolo[1,5- |
| | alpyrimidine-3,7-dicarboxamide |
| 40 | N7-indan-2-yl-N3-methyl-2-phenyl-pyrazolo[1,5-a]pyridine-3,7- |
| | dicarboxamide |
| 41 | N7-(3,3-difluorocyclobutyl)-5-methoxy-2-methyl-pyrazolo[1,5-a]pyrimidine- |
| | 3,7-dicarboxamide |
| 42 | N7-(3,3-difluorocyclobutyl)-2,5-dimethyl-pyrazolo[1,5-a]pyrimidine-3,7- |
| | dicarboxamide |
| 43 | N7-(3,3-difluorocyclobutyl)-2-tetrahydrofuran-2-yl-pyrazolo[1,5- |
| | alpyrimidine-3,7-dicarboxamide |
| 44 | N7-indan-2-yl-2-tetrahydrofuran-2-yl-pyrazolo[1,5-a]pyrimidine-3,7- |
| | dicarboxamide |
| 45 | N7-(3,3-difluorocyclobutyl)-2-tetrahydrofuran-3-yl-pyrazolo[1,5- |
| | alpyrimidine-3,7-dicarboxamide |
| 46 | N7-indan-2-yl-2-methyl-pyrazolo[1,5-a]pyrimidine-3,7-dicarboxamide |
| 47 | N7-chroman-3-yl-2-methyl-pyrazolo[1,5-a]pyrimidine-3,7-dicarboxamide |
| 48 | N7-(3,3-difluorocyclobutyl)-2-methyl-pyrazolo[1,5-a]pyrimidine-3,7- |
| | dicarboxamide |
| 49 | N7-(6,7-dihydro-5H-cyclopenta[b]pyridin-6-yl)-2-methyl-pyrazolo[1,5- |
| | alpyrimidine-3,7-dicarboxamide |
| 50 | N7-(cis-3-cyanocyclobutyl)-2-methyl-pyrazolo[1,5-a]pyrimidine-3,7- |
| | dicarboxamide |
| 51 | N7-(5-benzylindan-2-yl)-2-methyl-pyrazolo[1,5-a]pyrimidine-3,7- |
| | dicarboxamide |
| 52 | N7-(6,7-dihydro-5H-cyclopenta[c]pyridin-6-yl)-2-methyl-pyrazolo[1,5- |
| | alpyrimidine-3,7-dicarboxamide |
| 53 | 2-methyl-N7-[cis-3-(trifluoromethoxy)cyclobutyl]pyrazolo[1,5-a]pyrimidine- |
| | 3,7-dicarboxamide |
| 54 | N8-(3,3-difluorocyclobutyl)-2-(methoxymethyl)imidazo[1,2-b]pyridazine-3,8- |
| | dicarboxamide |
| 55 | 2-(methoxymethyl)-N8-[cis-3-(trifluoromethoxy)cyclobutyl]imidazo[1,2- |
| | b]pyridazine-3,8-dicarboxamide |
| 56 | N8-indan-2-yl-2-(methoxymethyl)imidazo[1,2-b]pyridazine-3,8- |
| | dicarboxamide |
| 57 | N7-indan-2-yl-2-(methoxymethyl)pyrazolo[1,5-a]pyrimidine-3,7- |
| | dicarboxamide |
| 58 | N7-(3,3-difluorocyclobutyl)-2-(methoxymethyl)pyrazolo[1,5-a]pyrimidine- |
| | 3,7-dicarboxamide |
| 59 | 2-(methoxymethyl)-N7-[5-(trifluoromethyl)-2-pyridyl]pyrazolo[1,5- |
| | alpyrimidine-3,7-dicarboxamide |
| 60 | N7-(6,7-dihydro-5H-cyclopenta[b]pyridin-6-yl)-2- |
| | (methoxymethyl)pyrazolo[1,5-a]pyrimidine-3,7-dicarboxamide |
| 61 | 2-(methoxymethyl)-N7-[cis-3-(trifluoromethoxy)cyclobutyl]pyrazolo[1,5- |
| | alpyrimidine-3,7-dicarboxamide |
| 62 | N7-[4-[(dimethylamino)methyl]phenyl]-2-(methoxymethyl)pyrazolo[1,5- |
| | alpyrimidine-3,7-dicarboxamide |
| 63 | 2-(methoxymethyl)-N7-[(1-methylpyrazol-4-yl)methyl]pyrazolo[1,5- |
| | alpyrimidine-3,7-dicarboxamide |
| 64 | N7-(4,6-dimethyl-2-pyridyl)-2-(methoxymethyl)pyrazolo[1,5-a]pyrimidine- |
| | 3,7-dicarboxamide |
| 65 | 2-(methoxymethyl)-N7-94-(trifluoromethyl)-2-pyridyl]pyrazolo[1,5- |
| | alpyrimidine-3,7-dicarboxamide |
| 66 | N7-(3,4-difluorophenyl)-2-(methoxymethyl)pyrazolo[1,5-a]pyrimidine-3,7- |
| | dicarboxamide |
| 67 | N7-(2,3-dihydrobenzofuran-5-ylmethyl)-2-(methoxymethyl)pyrazolo[1,5- |
| | alpyrimidine-3,7-dicarboxamide |
| 68 | N7-(2,4-difluorophenyl)-2-(methoxymethyl)pyrazolo[1,5-a]pyrimidine-3,7- |
| | dicarboxamide |
| 69 | N7-benzyl-2-(methoxymethyl)pyrazolo[1,5-a]pyrimidine-3,7-dicarboxamide |
| 70 | N7-[(4-fluorophenyl)methyl]-2-(methoxymethyl)pyrazolo[1,5-a]pyrimidine- |
| | 3,7-dicarboxamide |
| 71 | N7-[(3-fluorophenyl)methyl]-2-(methoxymethyl)pyrazolo[1,5-a]pyrimidine- |
| | 3,7-dicarboxamide |
| 72 | 2-(methoxymethyl)-N7-(p-tolylmethyl)pyrazolo[1,5-a]pyrimidine-3,7- |
| | dicarboxamide |
| 73 | 2-(methoxymethyl)-N7-(7-methoxytetralin-2-yl)pyrazolo[1,5-a]pyrimidine- |
| | 3,7-dicarboxamide |
| 74 | 2-(methoxymethyl)-N7-[(1R)-tetralin-1-yl]pyrazolo[1,5-a]pyrimidine-3,7- |
| | dicarboxamide |
| 75 | N7-(2,3-dihydrobenzofuran-3-yl)-2-(methoxymethyl)pyrazolo[1,5- |
| | alpyrimidine-3,7-dicarboxamide |
| 76 | N7-cyclohexyl-2-(methoxymethyl)pyrazolo[1,5-a]pyrimidine-3,7- |
| | dicarboxamide |
| 77 | N7-[(2,2-dimethylcyclopropyl)methyl]-2-(methoxymethyl)pyrazolo[1,5- |
| | alpyrimidine-3,7-dicarboxamide |
| 78 | N7-chroman-3-yl-2-(methoxymethyl)pyrazolo[1,5-a]pyrimidine-3,7- |
| | dicarboxamide |
| 79 | 2-(methoxymethyl)-N7-[(2S)-tetralin-2-yl]pyrazolo[1,5-a]pyrimidine-3,7- |
| | dicarboxamide |
| 80 | N7-(4,4-difluorocyclohexyl)-2-(methoxymethyl)pyrazolo[1,5-a]pyrimidine- |
| | 3,7-dicarboxamide |
| 81 | N7-(cyclopropylmethyl)-2-(methoxymethyl)pyrazolo[1,5-a]pyrimidine-3,7- |
| | dicarboxamide |
| 82 | N7-(7-fluorotetralin-2-yl)-2-(methoxymethyl)pyrazolo[1,5-a]pyrimidine-3,7- |
| | dicarboxamide |
| 83 | N7-[(2,4-difluorophenyl)methyl]-2-(methoxymethyl)pyrazolo[1,5- |
| | a]pyrimidine-3,7-dicarboxamide |
| 84 | 2-(methoxymethyl)-N7-(cis-4-methylcyclohexyl)pyrazolo[1,5-a]pyrimidine- |
| | 3,7-dicarboxamide |
| 85 | N7-(5,6-dihydro-4H-cyclopenta[c]thiophen-5-yl)-2- |
| | (methoxymethyl)pyrazolo[1,5-a]pyrimidine-3,7-dicarboxamide |
| 86 | 2-(methoxymethyl)-N7-(trans-3-methylcyclobutyl)pyrazolo[1,5-a]pyrimidine- |
| | 3,7-dicarboxamide |
| 87 | N7-(5-chloroindan-2-yl)-2-(methoxymethyl)pyrazolo[1,5-a]pyrimidine-3,7- |
| | dicarboxamide |
| 88 | N7-(7-bicyclo[4.2.0]octa-1,3,5-trienyl)-2-(methoxymethyl)pyrazolo[1,5- |
| | alpyrimidine-3,7-dicarboxamide |
| 89 | 2-(methoxymethyl)-7-[4-[3-(trifluoromethyl)-2-pyridyl]piperazine-1- |
| | carbonyl]pyrazolo[1,5-a]pyrimidine-3-carboxamide |
| 90 | N7-[1-(dimethylamino) cyclohexyl]methyl]-2-(methoxymethyl)pyrazolo[1,5- |
| | alpyrimidine-3,7-dicarboxamide |
| 91 | N7-(4-fluoroindan-2-yl)-2-(methoxymethyl)pyrazolo[1,5-a]pyrimidine-3,7- |
| | dicarboxamide |
| 92 | N7-(4,6-difluoroindan-2-yl)-2-(methoxymethyl)pyrazolo[1,5-a]pyrimidine- |
| | 3,7-dicarboxamide |
| 93 | 2-(methoxymethyl)-N7-(1,2,3,4-tetrahydroquinolin-3-yl)pyrazolo[1,5- |
| | alpyrimidine-3,7-dicarboxamide |
| 94 | N7-(trans-3-fluoro-3-methyl-cyclobutyl)-2-(methoxymethyl)pyrazolo[1,5- |
| | alpyrimidine-3,7-dicarboxamide |
| 95 | 7-(2,3-dihydro-1 H-isoindole-2-carbonyl)-2-(methoxymethyl)pyrazolo[1,5- |
| | alpyrimidine-3-carboxamide |
| 96 | N7-[2-(3-methylphenoxy)butyl]pyrazolo[1,5-a]pyrimidine-3,7-dicarboxamide |
| 97 | N7-[2-methyl-5-(pyridin-4-yl)phenyl]pyrazolo[1,5-a]pyrimidine-3,7- |
| | dicarboxamide |
| 98 | N7-[1-(5-methoxy-1H-1,3-benzodiazol-2-yl)ethyl]pyrazolo[1,5-a]pyrimidine- |
| | 3,7-dicarboxamide |
| 99 | N7-[1-(4-tert-butylphenoxy)propan-2-yl]pyrazolo[1,5-a]pyrimidine-3,7- |
| | dicarboxamide |
| 100 | N7-(3,3,3-trifluoropropyl)pyrazolo[1,5-a]pyrimidine-3,7-dicarboxamide |
| 101 | N7-[(4,4-difluorocyclohexyl)methyl]pyrazolo[1,5-a]pyrimidine-3,7- |
| | dicarboxamide |
| 102 | N7-(3,3-difluorocyclobutyl)pyrazolo[1,5-a]pyrimidine-3,7-dicarboxamide |
| 103 | N7-(4,4-difluorocyclohexyl)pyrazolo[1,5-a]pyrimidine-3,7-dicarboxamide |
| 104 | N8-(6,7-dihydro-5H-cyclopenta[b]pyridin-6-yl)-2- |
| | (methoxymethyl)imidazo[1,2-b]pyridazine-3,8-dicarboxamide |
| 105 | N8-(5,6-dihydro-4H-cyclopenta[c]thiophen-5-yl)-2- |
| | (methoxymethyl)imidazo[1,2-b]pyridazine-3,8-dicarboxamide |
| 106 | N8-(7-bicyclo[4.2.0]octa-1,3,5-trienyl)-2-(methoxymethyl)imidazo[1,2- |
| | b]pyridazine-3,8-dicarboxamide |
| 107 | N8-(3,3-difluoroindan-1-yl)-2-(methoxymethyl)imidazo[1,2-b]pyridazine-3,8- |
| | dicarboxamide |
| 108 | 2-(methoxymethyl)-N8-[(4S)-7-fluorochroman-4-yllimidazo[1,2-b]pyridazine- |
| | 3,8-dicarboxamide |
| 109 | N8-(4,4-difluorocyclohexyl)-2-(methoxymethyl)imidazo[1,2-b]pyridazine-3,8- |
| | dicarboxamide |
| 110 | N8-[(4-fluorophenyl)methyl]-2-(methoxymethyl)imidazo[1,2-b]pyridazine- |
| | 3,8-dicarboxamide |
| 111 | 2-cyclopropyl-N7-[cis-3-(trifluoromethoxy)cyclobutyl]pyrazolo[1,5- |
| | alpyrimidine-3,7-dicarboxamide |
| 112 | 2-cyclopropyl-N7-(3,3-difluoroindan-1-yl)pyrazolo[1,5-a]pyrimidine-3,7- |
| | dicarboxamide |
| 113 | 2-cyclopropyl-N7-[(4S)-7-fluorochroman-4-ylipyrazolo[1,5-a]pyrimidine-3,7- |
| | dicarboxamide |
| 114 | N7-(6-chlorochroman-3-yl)-2-(methoxymethyl)pyrazolo[1,5-a]pyrimidine- |
| | 3,7-dicarboxamide |
| 115 | 2-cyclopropyl-N7-(6,7-dihydro-5H-cyclopenta[b]pyridin-6-yl)pyrazolo[1,5- |
| | alpyrimidine-3,7-dicarboxamide |
| 116 | N7-(4,4-difluorotetralin-1-yl)-2-(methoxymethyl)pyrazolo[1,5-a]pyrimidine- |
| | 3,7-dicarboxamide |
| 117 | N7-(8-fluoro-3-quinolyl)-2-(methoxymethyl)pyrazolo[1,5-a]pyrimidine-3,7- |
| | dicarboxamide |
| 118 | 2-(methoxymethyl)-N7-(1-methylindol-2-yl)pyrazolo[1,5-a]pyrimidine-3,7- |
| | dicarboxamide |
| 119 | 2-(methoxymethyl)-N7-[(3R)-6,7-difluoro-2,3-dihydrobenzofuran-3- |
| | yllpyrazolo[1,5-a]pyrimidine-3,7-dicarboxamide |
| 120 | N7-[(3R)-5-fluoro-2,3-dihydrobenzofuran-3-yl]-2- |
| | (methoxymethyl)pyrazolo[1,5-a]pyrimidine-3,7-dicarboxamide |
| 121 | 2-(methoxymethyl)-N7-(2-methylindan-2-yl)pyrazolo[1,5-a]pyrimidine-3,7- |
| | dicarboxamide |
| 122 | N7-[1-(4-fluorophenyl)ethyl]-2-(methoxymethyl)pyrazolo[1,5-a]pyrimidine- |
| | 3,7-dicarboxamide |
| 123 | 2-(methoxymethyl)-N7-[(4S)-7-fluorochroman-4-yl]pyrazolo[1,5- |
| | alpyrimidine-3,7-dicarboxamide |
| 124 | N7-(2,2-difluoro-6-methyl-spiro[3.3]heptan-6-yl)-2- |
| | (methoxymethyl)pyrazolo[1,5-a]pyrimidine-3,7-dicarboxamide |
| 125 | 2-(methoxymethyl)-N7-[2,2,2-trifluoro-1-(4-fluorophenyl)ethyl]pyrazolo[1,5- |
| | alpyrimidine-3,7-dicarboxamide |
| 126 | N7-(2,2-difluorospiro[3.3]heptan-6-yl)-2-(methoxymethyl)pyrazolo[1,5- |
| | alpyrimidine-3,7-dicarboxamide |
| 127 | N7-[1-(4-fluorophenyl)cyclopropyl]-2-(methoxymethyl)pyrazolo[1,5- |
| | alpyrimidine-3,7-dicarboxamide |
| 128 | 2-(methoxymethyl)-N7-[(3R)-6-fluoro-2,3-dihydrobenzofuran-3- |
| | yl]pyrazolo[1,5-a]pyrimidine-3,7-dicarboxamide |
| 129 | N7-(3,3-difluoroindan-1-yl)-2-(methoxymethyl)pyrazolo[1,5-a]pyrimidine- |
| | 3,7-dicarboxamide |
| 130 | 2-cyclopropyl-N7-indan-2-yl-pyrazolo[1,5-a]pyrimidine-3,7-dicarboxamide |
| 131 | N7-(2,2-difluorospiro[3.3]heptan-7-yl)-2-(methoxymethyl)pyrazolo[1,5- |
| | alpyrimidine-3,7-dicarboxamide |
| 132 | 2-(methoxymethyl)-N7-[cis-4-(trifluoromethoxy)cyclohexyl]pyrazolo[1,5- |
| | alpyrimidine-3,7-dicarboxamide |
| 133 | N7-[(4,4-difluorocyclohexyl)methyl]-2-(methoxymethyl)pyrazolo[1,5- |
| | alpyrimidine-3,7-dicarboxamide |
| 134 | 2-cyclopropyl-N7-(5,6-dihydro-4H-cyclopenta[c]thiophen-5-yl)pyrazolo[1,5- |
| | alpyrimidine-3,7-dicarboxamide |
| 135 | 2-cyclopropyl-N7-(4,4-difluorocyclohexyl)pyrazolo[1,5-a]pyrimidine-3,7- |
| | dicarboxamide |
| 136 | 2-cyclopropyl-N7-(3,3-difluorocyclobutyl)pyrazolo[1,5-a]pyrimidine-3,7- |
| | dicarboxamide |
| 137 | 2-propyl-N7-[cis-3-(trifluoromethoxy)cyclobutyl]pyrazolo[1,5-a]pyrimidine- |
| | 3,7-dicarboxamide |
| 138 | N7-(7-bicyclo[4.2.0]octa-1,3,5-trienyl)-2-cyclopropyl-pyrazolo[1,5- |
| | alpyrimidine-3,7-dicarboxamide |
| 139 | N7-indan-2-yl-2-propyl-pyrazolo[1,5-a]pyrimidine-3,7-dicarboxamide |
| 140 | 2-cyclopropyl-N7-[(4-fluorophenyl)methyl]pyrazolo[1,5-a]pyrimidine-3,7- |
| | dicarboxamide |
| 141 | 2-(hydroxymethyl)-N7-[cis-3-(trifluoromethoxy)cyclobutyl]pEyrazolo[1,5- |
| | alpyrimidine-3,7-dicarboxamide |
| 142 | 2-(cyclopropylmethoxy)-N7-indan-2-yl-pyrazolo[1,5-a]pyrimidine-3,7- |
| | dicarboxamide |
| 143 | 7-(indan-2-ylcarbamoyl)-2-(methoxymethyl)pyrazolo[1,5-a]pyrimidine-3- |
| | carboxylic acid |
| 144 | N7-(6-bromochroman-3-yl)-2-(methoxymethyl)pyrazolo[1,5-a]pyrimidine- |
| | 3,7-dicarboxamide |
| 145 | 2-(methylamino)-N7-[cis-3-(trifluoromethoxy)cyclobutyl)pyrazolo[1,5- |
| | a]pyrimidine-3,7-dicarboxamide |
| 146 | N7-(6,7-dihydro-5H-cyclopenta[b]pyridin-6-yl)-2- |
| | (methoxymethyl)pyrazolo[1,5-a]pyrimidine-3,7-dicarboxamide |
| 147 | N7-(6,7-dihydro-5H-cyclopenta[b]pyridin-6-yl)-2- |
| | (methoxymethyl)pyrazolo[1,5-a]pyrimidine-3,7-dicarboxamide |
| 148 | 7-(indan-2-ylcarbamothioyl)-2-(methoxymethyl)pyrazolo[1,5-a]pyrimidine-3- |
| | carboxamide |
| 149 | 3-carbamothioyl-N-indan-2-yl-2-(methoxymethyl)pyrazolo[1,5-a]pyrimidine- |
| | 7-carboxamide |
| 150 | 2-cyclopropyl-N8-(4,4-difluorocyclohexyl)imidazo[1,2-b]pyridazine-3,8- |
| | dicarboxamide |
| 151 | 2-cyclopropyl-N8-[cis-3-(trifluoromethoxy)cyclobutyl]imidazo[1,2- |
| | b]pyridazine-3,8-dicarboxamide |
| 152 | N7-(1,1-difluoroindan-2-yl)-2-(methoxymethyl)pyrazolo[1,5-a]pyrimidine- |
| | 3,7-dicarboxamide |
| 153 | N7-(4,6-difluoro-2,3-dihydrobenzofuran-3-yl)-2- |
| | (methoxymethyl)pyrazolo[1,5-a]pyrimidine-3,7-dicarboxamide |
| 154 | 2-(methoxymethyl)-N7-(2,4,4-trifluorocyclohexyl)pyrazolo[1,5-a]pyrimidine- |
| | 3,7-dicarboxamide |
| 155 | 2-(methoxymethyl)-N7-(1-methylindan-1-yl)pyrazolo[1,5-a]pyrimidine-3,7- |
| | dicarboxamide |
| 156 | 7-cyclopropyl-N4-(4,4-difluorocyclohexyl)pyrazolo[5,1-c][1,2,4]triazine-4,8- |
| | dicarboxamide |
| 157 | 2-(trideuteriomethoxymethyl)-N7-[cis-3- |
| | (trifluoromethoxy)cyclobutyl\|pyrazolo[1,5-a]pyrimidine-3,7-dicarboxamide |
| 158 | 2-(fluoromethoxymethyl)-N7-[cis-3- |
| | (trifluoromethoxy)cyclobutyl]pyrazolo[1,5-a]pyrimidine-3,7-dicarboxamide |
| 159 | N7-indan-2-yl-2-(methylamino)pyrazolo[1,5-a]pyrimidine-3,7-dicarboxamide |
| 160 | N7-(6,7-dihydro-5H-cyclopenta[b]pyridin-6-yl)-2- |
| | (trideuteriomethoxymethyl)pyrazolo[1,5-a]pyrimidine-3,7-dicarboxamide |
| 161 | 2-(methoxymethyl)-N3-methyl-N7-[cis-3- |
| | (trifluoromethoxy)cyclobutyl]pyrazolo[1,5-a]pyrimidine-3,7-dicarboxamide |
| 162 | 2-(cyclopropylmethoxy)-N7-[cis-3-(trifluoromethoxy)cyclobutyl]pyrazolo[1,5- |
| | alpyrimidine-3,7-dicarboxamide |
| 163 | 2-cyclopropyl-N8-(6,7-dihydro-5H-cyclopenta[b]pyridin-6-yl)imidazo[1,2- |
| | bjpyridazine-3,8-dicarboxamide |
| 164 | N8-(4,6-difluoroindan-2-yl)-2-(methoxymethyl)imidazo[1,2-b]pyridazine-3,8- |
| | dicarboxamide |
| 165 | N7-(6,7-dihydro-5H-pyrrolo[1,2-a]imidazol-6-yl)-2- |
| | (methoxymethyl)pyrazolo[1,5-a]pyrimidine-3,7-dicarboxamide |
| 166 | N7-[(4R)-7-fluorochroman-4-yl]-2-(methoxymethyl)pyrazolo[1,5- |
| | alpyrimidine-3,7-dicarboxamide |
| 167 | N7-[(3S)-6-fluoro-2,3-dihydrobenzofuran-3-yl]-2- |
| | (methoxymethyl)pyrazolo[1,5-a]pyrimidine-3,7-dicarboxamide |
| 168 | N7-(1,1-dioxo-2,3-dihydrobenzothiophen-3-yl)-2- |
| | (methoxymethyl)pyrazolo[1,5-a]pyrimidine-3,7-dicarboxamide |
| 169 | N7-[(3R)-7-chloro-2,3-dihydrobenzofuran-3-yl]-2- |
| | (methoxymethyl)pyrazolo[1,5-a]pyrimidine-3,7-dicarboxamide |
| 170 | N7-(6,7-dihydro-5H-cyclopenta[c]pyridin-6-yl)-2- |
| | (methoxymethyl)pyrazolo[1,5-a]pyrimidine-3,7-dicarboxamide |
| 171 | N7-(3,4-dihydro-2H-pyrano[2,3-b]pyridin-4-yl)-2- |
| | (methoxymethyl)pyrazolo[1,5-a]pyrimidine-3,7-dicarboxamide |
| 172 | N7-[(4S)-chroman-4-yl]-2-(methoxymethyl)pyrazolo[1,5-a]pyrimidine-3,7- |
| | dicarboxamide |
| 173 | N7-(3,3-difluoroindan-1-yl)-2-(methoxymethyl)pyrazolo[1,5-a]pyrimidine- |
| | 3,7-dicarboxamide |
| 174 | N7-(3,3-difluoroindan-1-yl)-2-(methoxymethyl)pyrazolo[1,5-a]pyrimidine- |
| | 3,7-dicarboxamide |
| 175 | N7-[-4,4-difluorotetralin-1-yl]-2-(methoxymethyl)pyrazolo[1,5-a]pyrimidine- |
| | 3,7-dicarboxamide |
| 176 | N7-[-4,4-difluorotetralin-1-yl]-2-(methoxymethyl)pyrazolo[1,5-a]pyrimidine- |
| | 3,7-dicarboxamide |
| 177 | N7-(1,1-dioxo-2,3-dihydrobenzothiophen-3-yl)-2- |
| | (methoxymethyl)pyrazolo[1,5-a]pyrimidine-3,7-dicarboxamide |
| 178 | N7-[(3R)-7-chloro-2,3-dihydrobenzofuran-3-yl]-2- |
| | (methoxymethyl)pyrazolo[1,5-a]pyrimidine-3,7-dicarboxamide |
| 179 | N7-(6,7-dihydro-5H-cyclopenta[c]pyridin-6-yl)-2- |
| | (methoxymethyl)pyrazolo[1,5-a]pyrimidine-3,7-dicarboxamide |
| 180 | N7-(3,4-dihydro-2H-pyrano[2,3-b]pyridin-4-yl)-2- |
| | (methoxymethyl)pyrazolo[1,5-a]pyrimidine-3,7-dicarboxamide |
| 181 | N7-[(4S)-chroman-4-yl]-2-(methoxymethyl)pyrazolo[1,5-a]pyrimidine-3,7- |
| | dicarboxamide |
| 182 | N7-(6,7-difluorochroman-4-yl)-2-(methoxymethyl)pyrazolo[1,5-a]pyrimidine- |
| | 3,7-dicarboxamide |
| 183 | 2-(methoxymethyl)-N7-[(1S)-indan-1-yl\|pyrazolo[1,5-a]pyrimidine-3,7- |
| | dicarboxamide |
| 184 | N7-(2,3-dihydrofuro[2,3-b]pyridin-3-yl)-2-(methoxymethyl)pyrazolo[1,5- |
| | ajpyrimidine-3,7-dicarboxamide |
| 185 | N7-(5,7-difluorochroman-3-yl)-2-(methoxymethyl)pyrazolo[1,5-a]pyrimidine- |
| | 3,7-dicarboxamide |
| 186 | N7-(4,4-dimethyltetralin-1-yl)-2-(methoxymethyl)pyrazolo[1,5-a]pyrimidine- |
| | 3,7-dicarboxamide |
| 187 | 2-(methoxymethyl)-N7-(5,6,7,8-tetrahydroimidazo[1,2-a]pyridin-6- |
| | yl)pyrazolo[1,5-a]pyrimidine-3,7-dicarboxamide |
| 188 | 2-(methoxymethyl)-N7-(5,6,7,8-tetrahydroimidazo[1,5-a]pyridin-6- |
| | yl)pyrazolo[1,5-a]pyrimidine-3,7-dicarboxamide |
| 189 | N7-(1, 1-dioxo-3,4-dihydro-2H-thiochromen-4-yl)-2- |
| | (methoxymethyl)pyrazolo[1,5-a]pyrimidine-3,7-dicarboxamide |
| 190 | 2-(methoxymethyl)-N7-(4,5,6,7-tetrahydro-1,3-benzothiazol-6- |
| | yl)pyrazolo[1,5-a]pyrimidine-3,7-dicarboxamide |
| 191 | N7-(5,7-difluoro-2,3-dihydrobenzofuran-3-yl)-2- |
| | (methoxymethyl)pyrazolo[1,5-a]pyrimidine-3,7-dicarboxamide |
| 192 | N7-(7-fluoro-2,3-dihydrobenzofuran-3-yl)-2-(methoxymethyl)pyrazolo[1,5- |
| | alpyrimidine-3,7-dicarboxamide |
| 193 | 2-(methoxymethyl)-N7-[(1S)-tetralin-1-yi]pyrazolo[1,5-a]pyrimidine-3,7- |
| | dicarboxamide |
| 194 | N7-(5-fluoroindan-2-yl)-2-(methoxymethyl)pyrazolo[1,5-a]pyrimidine-3,7- |
| | dicarboxamide |
| 195 | N7-(5,6-difluoroindan-2-yl)-2-(methoxymethyl)pyrazolo[1,5-a]pyrimidine- |
| | 3,7-dicarboxamide |
| 196 | N7-(6-fluorochroman-3-yl)-2-(methoxymethyl)pyrazolo[1,5-a]pyrimidine-3,7- |
| | dicarboxamide |
| 197 | N7-[(4-hydroxyphenyl)methyl]-2-(methoxymethyl)pyrazolo[1,5-a]pyrimidine- |
| | 3,7-dicarboxamide |
| 198 | 2-(methoxymethyl)-N7-[(1S)-3,3-dimethylindan-1-yl]pyrazolo[1,5- |
| | alpyrimidine-3,7-dicarboxamide |
| 199 | 2-(methoxymethyl)-N7-[(2-methoxyphenyl)methyl]pyrazolo[1,5- |
| | alpyrimidine-3,7-dicarboxamide |
| 200 | N7-(cis-1-hydroxyindan-2-yl)-2-(methoxymethyl)pyrazolo[1,5-a]pyrimidine- |
| | 3,7-dicarboxamide |
| 201 | 2-(methoxymethyl)-N7-cis-1-methylindan-2-yl]pyrazolo[1,5-a]pyrimidine- |
| | 3,7-dicarboxamide |
| 202 | 2-(ethoxymethyl)-N7-indan-2-yl-pyrazolo[1,5-a]pyrimidine-3,7- |
| | dicarboxamide |
| 203 | 2-tetrahydrofuran-2-yl-N7-[cis-3-(trifluoromethoxy)cyclobutyljpyrazolo[1,5- |
| | alpyrimidine-3,7-dicarboxamide |
| 204 | 2-(hydroxymethyl)-N7-indan-2-yl-pyrazolo[1,5-a]pyrimidine-3,7- |
| | dicarboxamide |
| 205 | 2-(ethoxymethyl)-N7-[cis-3-(trifluoromethoxy)cyclobutyl]pyrazolo[1,5- |
| | alpyrimidine-3,7-dicarboxamide |
| 206 | 2-(methoxymethyl)-5-methyl-N7-[cis-3- |
| | (trifluoromethoxy)cyclobutyl]pyrazolo[1,5-a]pyrimidine-3,7-dicarboxamide |
| 207 | N7-(3,3-difluorocyclobutyl)-2-(hydroxymethyl)pyrazolo[1,5-a]pyrimidine-3,7- |
| | dicarboxamide |
| 208 | 2-cyclopropyl-N8-(5,6-dihydro-4H-cyclopenta[c]thiophen-5-yl)imidazo[1,2- |
| | b]pyridazine-3,8-dicarboxamide |
| 209 | 2-cyclopropyl-N8-(3,3-difluoroindan-1-yl)imidazo[1,2-b]pyridazine-3,8- |
| | dicarboxamide |
| 210 | N8-(7-bicyclo[4.2.0]octa-1,3,5-trienyl)-2-cyclopropyl-imidazo[1,2- |
| | bjpyridazine-3,8-dicarboxamide |
| 211 | 2-cyclopropyl-N8-[(4S)-7-fluorochroman-4-yllimidazo[1,2-b]pyridazine-3,8- |
| | dicarboxamide |
| 212 | N4-(4,4-difluorocyclohexyl)-6-(methoxymethyl)imidazo[2,1-f][1,2,4]triazine- |
| | 4,7-dicarboxamide |
| 213 | N7-(3,3-difluorocyclobutyl)-2-(1-hydroxy-1-methyl-ethyl)pyrazolo[1,5- |
| | alpyrimidine-3,7-dicarboxamide |
| 214 | N7-indan-2-yl-2-(methylsulfanylmethyl)pyrazolo[1,5-a]pyrimidine-3,7- |
| | dicarboxamide |
| 215 | N7-indan-2-yl-2-(methylsulfonylmethyl)pyrazolo[1,5-a]pyrimidine-3,7- |
| | dicarboxamide |
| 216 | 2-(cyclopropoxymethyl)-N7-[cis-3-(trifluoromethoxy)cyclobutyl]pyrazolo[1,5- |
| | alpyrimidine-3,7-dicarboxamide |
| 217 | 2-(methylsulfanylmethyl)-N7-[cis-3- |
| | (trifluoromethoxy)cyclobutyl]pyrazolo[1,5-a]pyrimidine-3,7-dicarboxamide |
| 218 | N8-(4,4-difluorocyclohexyl)-2-(methoxymethyl)-6-methyl-imidazo[1,2- |
| | b]pyridazine-3,8-dicarboxamide |
| 219 | 2-(methoxymethyl)-N8-[(4R)-1,1-dioxo-3,4-dihydro-2H-thiochromen-4- |
| | yllimidazo[1,2-b]pyridazine-3,8-dicarboxamide |
| 220 | 2-(methoxymethyl)-N8-[(4S)-1, 1-dioxo-3,4-dihydro-2H-thiochromen-4- |
| | yllimidazo[1,2-b]pyridazine-3,8-dicarboxamide |
| 221 | N8-(1, 1-dioxo-3,4-dihydro-2H-thiochromen-4-yl)-2- |
| | (methoxymethyl)imidazo[1,2-b]pyridazine-3,8-dicarboxamide |
| 222 | 2-cyclopropyl-N8-(1,1-dioxo-2,3-dihydrobenzothiophen-3-yl)imidazo[1,2- |
| | b]pyridazine-3,8-dicarboxamide |
| 223 | N8-(1,1-dioxo-2,3-dihydrobenzothiophen-3-yl)-2- |
| | (methoxymethyl)imidazo[1,2-b]pyridazine-3,8-dicarboxamide |
| 224 | 2-(methoxymethyl)-N7-[trans-3-(trifluoromethoxy)cyclobutyl]pyrazolo[1,5- |
| | ajpyrimidine-3,7-dicarboxamide |
| 225 | N7-(1,1-dioxothietan-3-yl)-2-(methoxymethyl) pyrazolo[1,5-a] pyrimidine- |
| | 3,7-dicarboxamide |
| 226 | N7-(4,4-difluorotetralin-1-yl)-2-(methoxymethyl)-5-methyl-pyrazolo[1,5- |
| | alpyrimidine-3,7-dicarboxamide |
| 227 | N7-indan-2-yl-2-(methoxymethyl)-5-methyl-pyrazolo[1,5-a]pyrimidine-3,7. |
| | dicarboxamide |
| 228 | N7-(1,1-dioxo-2,3-dihydrobenzothiophen-3-yl)-2-isopropyl-pyrazolo[1,5- |
| | alpyrimidine-3,7-dicarboxamide |
| 229 | 2-cyclopropyl-N7-(1,1-dioxo-3,4-dihydro-2H-thiochromen-4-yl)pyrazolo[1,5- |
| | alpyrimidine-3,7-dicarboxamide |
| 230 | N7-(1, 1-dioxo-3,4-dihydro-2H-thiochromen-4-yl)-2-isopropyl-pyrazolo[1,5- |
| | alpyrimidine-3,7-dicarboxamide |
| 231 | 2-cyclopropyl-N7-(1,1-dioxo-2,3-dihydrobenzothiophen-3-yl)pyrazolo[1,5- |
| | alpyrimidine-3,7-dicarboxamide |
| 232 | N7-(3,3-difluoroindan-1-yl)-2-(methoxymethyl)-5-methyl-pyrazolo[1,5- |
| | alpyrimidine-3,7-dicarboxamide |
| 233 | N7-[(4R)-1,1-dioxo-3,4-dihydro-2H-thiochromen-4-yl]-2- |
| | (methoxymethyl)pyrazolo[1,5-a]pyrimidine-3,7-dicarboxamide |
| 234 | N7-[(4S)-1,1-dioxo-3,4-dihydro-2H-thiochromen-4-yl]-2- |
| | (methoxymethyl)pyrazolo[1,5-a]pyrimidine-3,7-dicarboxamide |
| 235 | 2-cyclopropyl-N7-[(4R)-1, 1 -dioxo-3,4-dihydro-2H-thiochromen-4- |
| | yl]pyrazolo[1,5-a]pyrimidine-3,7-dicarboxamide |
| 236 | 2-cyclopropyl-N7-[(4S)-1, 1-dioxo-3,4-dihydro-2H-thiochromen-4-yl] |
| | pyrazolo[1,5-a]pyrimidine-3,7-dicarboxamide |
| 237 | 2-(methoxymethyl)-N7-(3,3,6-trifluoroindan-1-yl) pyrazolo[1,5-a] pyrimidine- |
| | 3,7-dicarboxamide |
| 238 | N7-[(3S)-6,7-difluoro-2,3-dihydrobenzofuran-3-yi]-2- |
| | (methoxymethyl)pyrazolo[1,5-a]pyrimidine-3,7-dicarboxamide |
| 239 | 2-(methoxymethyl)-N7-(4,4,6-trifluorotetralin-1-yl)pyrazolo[1,5-a]pyrimidine- |
| | 3,7-dicarboxamide |
| 240 | N7-(4,4-difluorocyclohexyl)-2-(methoxymethyl)-5-methyl-pyrazolo[1,5- |
| | alpyrimidine-3,7-dicarboxamide |
| 241 | N7-(1,1-dioxo-3,4-dihydro-2H-thiochromen-4-yl)-2-(methoxymethyl)-5- |
| | methyl-pyrazolo[1,5-a]pyrimidine-3,7-dicarboxamide |
| 242 | 2-cyclopropyl-N7-(1,1-dioxo-3,4-dihydro-2H-thiochromen-4-yl)-5-methyl- |
| | pyrazolo[1,5-a]pyrimidine-3,7-dicarboxamide |

In embodiments, the compound may be selected from compounds 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, and 242 of Table 1. In embodiments, the compound may be selected from compounds 1, 2, 4, 5, 6, 7, 8, 9, 11, 14, 15, 18, 19, 20, 21, 22, 25, 26, 27, 28, 29, 30, 31, 32, 33, 34, 35, 36, 37, 38, 39, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, and 242 of Table 1. In embodiments, the compound may be selected from compounds 1, 2, 4, 5, 6, 9, 14, 18, 21, 22, 25, 26, 27, 29, 30, 32, 33, 34, 35, 36, 37, 39, 41, 42, 43, 44, 45, 46, 47, 48, 49, 50, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 91, 92, 93, 94, 95, 97, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, and 242 of Table 1. In embodiments, the compound may be selected from compounds 1, 2, 4, 6, 21, 26, 27, 33, 34, 37, 42, 43, 44, 46, 48, 53, 54, 55, 56, 57, 58, 59, 60, 61, 64, 65, 66, 67, 68, 69, 70, 71, 72, 73, 74, 75, 76, 78, 79, 80, 81, 82, 83, 85, 86, 87, 88, 91, 92, 93, 94, 104, 105, 106, 107, 108, 109, 110, 111, 112, 113, 114, 115, 116, 117, 118, 119, 120, 121, 122, 123, 126, 127, 128, 129, 130, 132, 133, 134, 135, 136, 137, 138, 139, 140, 141, 142, 143, 144, 145, 146, 147, 148, 149, 150, 151, 152, 153, 154, 155, 156, 157, 158, 159, 160, 162, 163, 164, 165, 166, 167, 168, 169, 170, 171, 172, 173, 174, 175, 176, 177, 178, 179, 180, 181, 182, 183, 184, 185, 189, 190, 191, 192, 193, 194, 195, 196, 198, 199, 200, 201, 202, 203, 204, 205, 206, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, and 242 of Table 1. In embodiments, the compound may be selected from compounds 27, 33, 34, 37, 46, 54, 56, 57, 58, 59, 60, 61, 64, 66, 68, 70, 72, 73, 74, 75, 79, 80, 82, 83, 85, 87, 88, 91, 92, 93, 105, 106, 107, 108, 109, 110, 111, 112, 113, 114, 115, 116, 117, 118, 119, 120, 123, 128, 129, 130, 133, 134, 135, 136, 137, 138, 139, 140, 141, 142, 144, 145, 147, 148, 150, 151, 152, 153, 157, 158, 159, 160, 163, 164, 166, 167, 168, 169, 170, 173, 174, 175, 176, 177, 178, 179, 182, 183, 184, 189, 191, 192, 193, 194, 195, 196, 200, 201, 202, 208, 209, 210, 211, 214, 216, 217, 219, 222, 223, 225, 226, 227, 228, 229, 230, 231, 232, 233, 234, 235, 236, 237, 238, 239, 240, and 242 of Table 1.

In another aspect of this disclosure, there is provided a compound of formula (1) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, for use in medicine.

In embodiments, the compound of formula (1), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the compound of formula (1), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of fibrotic disease or inflammatory disease.

In embodiments, the compound of formula (1), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound orally, topically, by inhalation, by intranasal administration, by intracerebroventricular; or systemically by intravenous, intraperitoneal, subcutaneous, or intramuscular injection.

In embodiments, the compound of formula (1), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (1), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, as a monotherapy or in combination with a secondary agent such as immune checkpoint inhibitors, DNA damaging agents, or ionising radiation.

In embodiments, the compound of formula (1), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (1), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the compound of formula (1), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, simultaneously, sequentially or separately from the one or more additional therapeutic agent.

In embodiments, the compound of formula (1), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (1), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more therapy selected from: radiation therapy, chemotherapy, immunotherapy and targeted therapy.

In another aspect of this disclosure, there is provided a compound of formula (2) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, for use in medicine.

In embodiments, the compound of formula (2), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the compound of formula (2), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of fibrotic disease or inflammatory disease.

In embodiments, the compound of formula (2), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound orally, topically, by inhalation, by intranasal administration, by intracerebroventricular; or systemically by intravenous, intraperitoneal, subcutaneous, or intramuscular injection.

In embodiments, the compound of formula (2), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (2), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, as a monotherapy or in combination with a secondary agent such as immune checkpoint inhibitors, DNA damaging agents, or ionising radiation.

In embodiments, the compound of formula (2), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (2), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the compound of formula (2), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, simultaneously, sequentially or separately from the one or more additional therapeutic agent.

In embodiments, the compound of formula (2), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (2), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more therapy selected from: radiation therapy, chemotherapy, immunotherapy and targeted therapy.

In another aspect of this disclosure, there is provided a compound of formula (2a) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, for use in medicine.

In embodiments, the compound of formula (2a), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the compound of formula (2a), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of fibrotic disease or inflammatory disease.

In embodiments, the compound of formula (2a), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound orally, topically, by inhalation, by intranasal administration, by intracerebroventricular; or systemically by intravenous, intraperitoneal, subcutaneous, or intramuscular injection.

In embodiments, the compound of formula (2a), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (2a), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, as a monotherapy or in combination with a secondary agent such as immune checkpoint inhibitors, DNA damaging agents, or ionising radiation.

In embodiments, the compound of formula (2a), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (2a), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the compound of formula (2a), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, simultaneously, sequentially or separately from the one or more additional therapeutic agent.

In embodiments, the compound of formula (2a), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (2a), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more therapy selected from: radiation therapy, chemotherapy, immunotherapy and targeted therapy.

In another aspect of this disclosure, there is provided a compound of formula (2aa) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, for use in medicine.

In embodiments, the compound of formula (2aa), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the compound of formula (2aa), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of fibrotic disease or inflammatory disease.

In embodiments, the compound of formula (2aa), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound orally, topically, by inhalation, by intranasal administration, by intracerebroventricular; or systemically by intravenous, intraperitoneal, subcutaneous, or intramuscular injection.

In embodiments, the compound of formula (2aa), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (2aa), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, as a monotherapy or in combination with a secondary agent such as immune checkpoint inhibitors, DNA damaging agents, or ionising radiation.

In embodiments, the compound of formula (2aa), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (2aa), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the compound of formula (2aa), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, simultaneously, sequentially or separately from the one or more additional therapeutic agent.

In embodiments, the compound of formula (2aa), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (2aa), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more therapy selected from: radiation therapy, chemotherapy, immunotherapy and targeted therapy.

In another aspect of this disclosure, there is provided a compound of formula (2ab) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, for use in medicine.

In embodiments, the compound of formula (2ab), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the compound of formula (2ab), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of fibrotic disease or inflammatory disease.

In embodiments, the compound of formula (2ab), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound orally, topically, by inhalation, by intranasal administration, by intracerebroventricular; or systemically by intravenous, intraperitoneal, subcutaneous, or intramuscular injection.

In embodiments, the compound of formula (2ab), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (2ab), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, as a monotherapy or in combination with a secondary agent such as immune checkpoint inhibitors, DNA damaging agents, or ionising radiation.

In embodiments, the compound of formula (2ab), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (2ab), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the compound of formula (2ab), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, simultaneously, sequentially or separately from the one or more additional therapeutic agent.

In embodiments, the compound of formula (2ab), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (2ab), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more therapy selected from: radiation therapy, chemotherapy, immunotherapy and targeted therapy.

In another aspect of this disclosure, there is provided a compound of formula (2ac) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, for use in medicine.

In embodiments, the compound of formula (2ac), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the compound of formula (2ac), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of fibrotic disease or inflammatory disease.

In embodiments, the compound of formula (2ac), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound orally, topically, by inhalation, by intranasal administration, by intracerebroventricular; or systemically by intravenous, intraperitoneal, subcutaneous, or intramuscular injection.

In embodiments, the compound of formula (2ac), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (2ac), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, as a monotherapy or in combination with a secondary agent such as immune checkpoint inhibitors, DNA damaging agents, or ionising radiation.

In embodiments, the compound of formula (2ac), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (2ac), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the compound of formula (2ac), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, simultaneously, sequentially or separately from the one or more additional therapeutic agent.

In embodiments, the compound of formula (2ac), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (2ac), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more therapy selected from: radiation therapy, chemotherapy, immunotherapy and targeted therapy.

In another aspect of this disclosure, there is provided a compound of formula (2ad) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, for use in medicine.

In embodiments, the compound of formula (2ad), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the compound of formula (2ad), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of fibrotic disease or inflammatory disease.

In embodiments, the compound of formula (2ad), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound orally, topically, by inhalation, by intranasal administration, by intracerebroventricular; or systemically by intravenous, intraperitoneal, subcutaneous, or intramuscular injection.

In embodiments, the compound of formula (2ad), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (2ad), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, as a monotherapy or in combination with a secondary agent such as immune checkpoint inhibitors, DNA damaging agents, or ionising radiation.

In embodiments, the compound of formula (2ad), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (2ad), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the compound of formula (2ad), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, simultaneously, sequentially or separately from the one or more additional therapeutic agent.

In embodiments, the compound of formula (2ad), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (2ad), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more therapy selected from: radiation therapy, chemotherapy, immunotherapy and targeted therapy.

In another aspect of this disclosure, there is provided a compound of formula (2b) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, for use in medicine.

In embodiments, the compound of formula (2b), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the compound of formula (2b), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of fibrotic disease or inflammatory disease.

In embodiments, the compound of formula (2b), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound orally, topically, by inhalation, by intranasal administration, by intracerebroventricular; or systemically by intravenous, intraperitoneal, subcutaneous, or intramuscular injection.

In embodiments, the compound of formula (2b), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (2b), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, as a monotherapy or in combination with a secondary agent such as immune checkpoint inhibitors, DNA damaging agents, or ionising radiation.

In embodiments, the compound of formula (2b), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (2b), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the compound of formula (2b), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, simultaneously, sequentially or separately from the one or more additional therapeutic agent.

In embodiments, the compound of formula (2b), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (2b), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more therapy selected from: radiation therapy, chemotherapy, immunotherapy and targeted therapy.

In another aspect of this disclosure, there is provided a compound of formula (3) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, for use in medicine.

In embodiments, the compound of formula (3), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the compound of formula (3), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of fibrotic disease or inflammatory disease.

In embodiments, the compound of formula (3), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound orally, topically, by inhalation, by intranasal administration, by intracerebroventricular; or systemically by intravenous, intraperitoneal, subcutaneous, or intramuscular injection.

In embodiments, the compound of formula (3), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (3), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, as a monotherapy or in combination with a secondary agent such as immune checkpoint inhibitors, DNA damaging agents, or ionising radiation.

In embodiments, the compound of formula (3), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (3), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the compound of formula (3), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, simultaneously, sequentially or separately from the one or more additional therapeutic agent.

In embodiments, the compound of formula (3), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (3), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more therapy selected from: radiation therapy, chemotherapy, immunotherapy and targeted therapy.

In another aspect of this disclosure, there is provided a compound of formula (4) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, for use in medicine.

In embodiments, the compound of formula (4), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the compound of formula (4), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of fibrotic disease or inflammatory disease.

In embodiments, the compound of formula (4), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound orally, topically, by inhalation, by intranasal administration, by intracerebroventricular; or systemically by intravenous, intraperitoneal, subcutaneous, or intramuscular injection.

In embodiments, the compound of formula (4), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (4), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, as a monotherapy or in combination with a secondary agent such as immune checkpoint inhibitors, DNA damaging agents, or ionising radiation.

In embodiments, the compound of formula (4), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (4), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the compound of formula (4), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, simultaneously, sequentially or separately from the one or more additional therapeutic agent.

In embodiments, the compound of formula (4), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (4), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more therapy selected from: radiation therapy, chemotherapy, immunotherapy and targeted therapy.

In another aspect of this disclosure, there is provided a compound of formula (4a) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, for use in medicine.

In embodiments, the compound of formula (4a), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the compound of formula (4a), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of fibrotic disease or inflammatory disease.

In embodiments, the compound of formula (4a), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound orally, topically, by inhalation, by intranasal administration, by intracerebroventricular; or systemically by intravenous, intraperitoneal, subcutaneous, or intramuscular injection.

In embodiments, the compound of formula (4a), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (4a), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, as a monotherapy or in combination with a secondary agent such as immune checkpoint inhibitors, DNA damaging agents, or ionising radiation.

In embodiments, the compound of formula (4a), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (4a), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the compound of formula (4a), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, simultaneously, sequentially or separately from the one or more additional therapeutic agent.

In embodiments, the compound of formula (4a), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (4a), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more therapy selected from: radiation therapy, chemotherapy, immunotherapy and targeted therapy.

In another aspect of this disclosure, there is provided a compound of formula (4b) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, for use in medicine.

In embodiments, the compound of formula (4b), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the compound of formula (4b), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of fibrotic disease or inflammatory disease.

In embodiments, the compound of formula (4b), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound orally, topically, by inhalation, by intranasal administration, by intracerebroventricular; or systemically by intravenous, intraperitoneal, subcutaneous, or intramuscular injection.

In embodiments, the compound of formula (4b), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (4b), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, as a monotherapy or in combination with a secondary agent such as immune checkpoint inhibitors, DNA damaging agents, or ionising radiation.

In embodiments, the compound of formula (4b), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (4b), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the compound of formula (4b), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, simultaneously, sequentially or separately from the one or more additional therapeutic agent.

In embodiments, the compound of formula (4b), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (4b), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more therapy selected from: radiation therapy, chemotherapy, immunotherapy and targeted therapy.

In another aspect of this disclosure, there is provided a compound of formula (4c) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, for use in medicine.

In embodiments, the compound of formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the compound of formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in the treatment or prophylaxis of fibrotic disease or inflammatory disease.

In embodiments, the compound of formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound orally, topically, by inhalation, by intranasal administration, by intracerebroventricular; or systemically by intravenous, intraperitoneal, subcutaneous, or intramuscular injection.

In embodiments, the compound of formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, as a monotherapy or in combination with a secondary agent such as immune checkpoint inhibitors, DNA damaging agents, or ionising radiation.

In embodiments, the compound of formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the compound of formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, simultaneously, sequentially or separately from the one or more additional therapeutic agent.

In embodiments, the compound of formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for use in a method comprising administering the compound of formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more therapy selected from: radiation therapy, chemotherapy, immunotherapy and targeted therapy.

In another aspect of this disclosure, there is provided a pharmaceutical composition comprising a compound of formula (1) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier.

In another aspect of this disclosure, there is provided a pharmaceutical composition comprising a compound of formula (1) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier, for use in medicine.

In embodiments, the pharmaceutical composition is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the pharmaceutical composition is for use in the treatment or prophylaxis of fibrotic disease or inflammatory disease.

In embodiments, the pharmaceutical composition is for use in a method comprising administering the compound orally, topically, by inhalation, by intranasal administration, by intracerebroventricular; or systemically by intravenous, intraperitoneal, subcutaneous, or intramuscular injection.

In embodiments, the pharmaceutical composition is for use in a method comprising administering the pharmaceutical composition as a monotherapy or in combination with a secondary agent such as immune checkpoint inhibitors, DNA damaging agents, or ionising radiation.

In embodiments, the pharmaceutical composition is for use in a method comprising administering the pharmaceutical composition in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the pharmaceutical composition simultaneously, sequentially or separately from the one or more additional therapeutic agent.

In embodiments, the pharmaceutical composition is for use in a method comprising administering the pharmaceutical composition in combination with one or more therapy selected from: radiation therapy, chemotherapy, immunotherapy and targeted therapy.

In another aspect of this disclosure, there is provided a pharmaceutical composition comprising a compound of formula (2) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier.

In another aspect of this disclosure, there is provided a pharmaceutical composition comprising a compound of formula (2) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier, for use in medicine.

In embodiments, the pharmaceutical composition is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the pharmaceutical composition is for use in the treatment or prophylaxis of fibrotic disease or inflammatory disease.

In embodiments, the pharmaceutical composition is for use in a method comprising administering the compound orally, topically, by inhalation, by intranasal administration, by intracerebroventricular; or systemically by intravenous, intraperitoneal, subcutaneous, or intramuscular injection.

In embodiments, the pharmaceutical composition is for use in a method comprising administering the pharmaceutical composition as a monotherapy or in combination with a secondary agent such as immune checkpoint inhibitors, DNA damaging agents, or ionising radiation.

In embodiments, the pharmaceutical composition is for use in a method comprising administering the pharmaceutical composition in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the pharmaceutical composition simultaneously, sequentially or separately from the one or more additional therapeutic agent.

In embodiments, the pharmaceutical composition is for use in a method comprising administering the pharmaceutical composition in combination with one or more therapy selected from: radiation therapy, chemotherapy, immunotherapy and targeted therapy.

In another aspect of this disclosure, there is provided a pharmaceutical composition comprising a compound of formula (2a) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier.

In another aspect of this disclosure, there is provided a pharmaceutical composition comprising a compound of formula (2a) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier, for use in medicine.

In embodiments, the pharmaceutical composition is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the pharmaceutical composition is for use in the treatment or prophylaxis of fibrotic disease or inflammatory disease.

In embodiments, the pharmaceutical composition is for use in a method comprising administering the compound orally, topically, by inhalation, by intranasal administration, by intracerebroventricular; or systemically by intravenous, intraperitoneal, subcutaneous, or intramuscular injection.

In embodiments, the pharmaceutical composition is for use in a method comprising administering the pharmaceutical composition as a monotherapy or in combination with a secondary agent such as immune checkpoint inhibitors, DNA damaging agents, or ionising radiation.

In embodiments, the pharmaceutical composition is for use in a method comprising administering the pharmaceutical composition in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the pharmaceutical composition simultaneously, sequentially or separately from the one or more additional therapeutic agent.

In embodiments, the pharmaceutical composition is for use in a method comprising administering the pharmaceutical composition in combination with one or more therapy selected from: radiation therapy, chemotherapy, immunotherapy and targeted therapy.

In another aspect of this disclosure, there is provided a pharmaceutical composition comprising a compound of formula (2aa) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier.

In another aspect of this disclosure, there is provided a pharmaceutical composition comprising a compound of formula (2aa) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier, for use in medicine.

In embodiments, the pharmaceutical composition is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the pharmaceutical composition is for use in the treatment or prophylaxis of fibrotic disease or inflammatory disease.

In embodiments, the pharmaceutical composition is for use in a method comprising administering the compound orally, topically, by inhalation, by intranasal administration, by intracerebroventricular; or systemically by intravenous, intraperitoneal, subcutaneous, or intramuscular injection.

In embodiments, the pharmaceutical composition is for use in a method comprising administering the pharmaceutical composition as a monotherapy or in combination with a secondary agent such as immune checkpoint inhibitors, DNA damaging agents, or ionising radiation.

In embodiments, the pharmaceutical composition is for use in a method comprising administering the pharmaceutical composition in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the pharmaceutical composition simultaneously, sequentially or separately from the one or more additional therapeutic agent.

In embodiments, the pharmaceutical composition is for use in a method comprising administering the pharmaceutical composition in combination with one or more therapy selected from: radiation therapy, chemotherapy, immunotherapy and targeted therapy.

In another aspect of this disclosure, there is provided a pharmaceutical composition comprising a compound of formula (2ab) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier.

In another aspect of this disclosure, there is provided a pharmaceutical composition comprising a compound of formula (2ab) as described above, or a stereoisomer or mixture of stereoisomers including diastereomers and enantiomers, tautomer, isotopic form, pharmaceutically acceptable salt, solvate, prodrug, or pharmaceutically active metabo lite thereof, or combinations thereof, and at least one pharmaceutically acceptable diluent, excipient or carrier, for use in medicine.

In embodiments, the pharmaceutical composition is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the pharmaceutical composition is for use in the treatment or prophylaxis of fibrotic disease or inflammatory disease.

In embodiments, the pharmaceutical composition is for use in a method comprising administering the compound orally, topically, by inhalation, by intranasal administration, by intracerebroventricular; or systemically by intravenous, intraperitoneal, subcutaneous, or intramuscular injection.

In embodiments, the pharmaceutical composition is for use in a method comprising administering the pharmaceutical composition as a monotherapy or in combination with a secondary agent such as immune checkpoint inhibitors, DNA damaging agents, or ionising radiation.

In embodiments, the pharmaceutical composition is for use in a method comprising administering the pharmaceutical composition in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the pharmaceutical composition simultaneously, sequentially or separately from the one or more additional therapeutic agent.

In embodiments, the pharmaceutical composition is for use in a method comprising administering the pharmaceutical composition in combination with one or more therapy selected from: radiation therapy, chemotherapy, immunotherapy and targeted therapy.

In another aspect of this disclosure, there is provided a pharmaceutical composition comprising a compound of formula (2ac) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier.

In another aspect of this disclosure, there is provided a pharmaceutical composition comprising a compound of formula (2ac) as described above, or a stereoisomer or mixture of stereoisomers including diastereomers and enantiomers, tautomer, isotopic form, pharmaceutically acceptable salt, solvate, prodrug, or pharmaceutically active metabo lite thereof, or combinations thereof, and at least one pharmaceutically acceptable diluent, excipient or carrier, for use in medicine.

In embodiments, the pharmaceutical composition is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the pharmaceutical composition is for use in the treatment or prophylaxis of fibrotic disease or inflammatory disease.

In embodiments, the pharmaceutical composition is for use in a method comprising administering the compound orally, topically, by inhalation, by intranasal administration, by intracerebroventricular; or systemically by intravenous, intraperitoneal, subcutaneous, or intramuscular injection.

In embodiments, the pharmaceutical composition is for use in a method comprising administering the pharmaceutical composition as a monotherapy or in combination with a secondary agent such as immune checkpoint inhibitors, DNA damaging agents, or ionising radiation.

In embodiments, the pharmaceutical composition is for use in a method comprising administering the pharmaceutical composition in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the pharmaceutical composition simultaneously, sequentially or separately from the one or more additional therapeutic agent.

In embodiments, the pharmaceutical composition is for use in a method comprising administering the pharmaceutical composition in combination with one or more therapy selected from: radiation therapy, chemotherapy, immunotherapy and targeted therapy.

In another aspect of this disclosure, there is provided a pharmaceutical composition comprising a compound of formula (2b) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier.

In another aspect of this disclosure, there is provided a pharmaceutical composition comprising a compound of formula (2b) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier, for use in medicine.

In embodiments, the pharmaceutical composition is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the pharmaceutical composition is for use in the treatment or prophylaxis of fibrotic disease or inflammatory disease.

In embodiments, the pharmaceutical composition is for use in a method comprising administering the compound orally, topically, by inhalation, by intranasal administration, by intracerebroventricular; or systemically by intravenous, intraperitoneal, subcutaneous, or intramuscular injection.

In embodiments, the pharmaceutical composition is for use in a method comprising administering the pharmaceutical composition as a monotherapy or in combination with a secondary agent such as immune checkpoint inhibitors, DNA damaging agents, or ionising radiation.

In embodiments, the pharmaceutical composition is for use in a method comprising administering the pharmaceutical composition in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the pharmaceutical composition simultaneously, sequentially or separately from the one or more additional therapeutic agent.

In embodiments, the pharmaceutical composition is for use in a method comprising administering the pharmaceutical composition in combination with one or more therapy selected from: radiation therapy, chemotherapy, immunotherapy and targeted therapy.

In another aspect of this disclosure, there is provided a pharmaceutical composition comprising a compound of formula (3) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier.

In another aspect of this disclosure, there is provided a pharmaceutical composition comprising a compound of formula (3) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier, for use in medicine.

In embodiments, the pharmaceutical composition is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the pharmaceutical composition is for use in the treatment or prophylaxis of fibrotic disease or inflammatory disease.

In embodiments, the pharmaceutical composition is for use in a method comprising administering the compound orally, topically, by inhalation, by intranasal administration, by intracerebroventricular; or systemically by intravenous, intraperitoneal, subcutaneous, or intramuscular injection.

In embodiments, the pharmaceutical composition is for use in a method comprising administering the pharmaceutical composition as a monotherapy or in combination with a secondary agent such as immune checkpoint inhibitors, DNA damaging agents, or ionising radiation.

In embodiments, the pharmaceutical composition is for use in a method comprising administering the pharmaceutical composition in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the pharmaceutical composition simultaneously, sequentially or separately from the one or more additional therapeutic agent.

In embodiments, the pharmaceutical composition is for use in a method comprising administering the pharmaceutical composition in combination with one or more therapy selected from: radiation therapy, chemotherapy, immunotherapy and targeted therapy.

In another aspect of this disclosure, there is provided a pharmaceutical composition comprising a compound of formula (4) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier.

In another aspect of this disclosure, there is provided a pharmaceutical composition comprising a compound of formula (4) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier, for use in medicine.

In embodiments, the pharmaceutical composition is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the pharmaceutical composition is for use in the treatment or prophylaxis of fibrotic disease or inflammatory disease.

In embodiments, the pharmaceutical composition is for use in a method comprising administering the compound orally, topically, by inhalation, by intranasal administration, by intracerebroventricular; or systemically by intravenous, intraperitoneal, subcutaneous, or intramuscular injection.

In embodiments, the pharmaceutical composition is for use in a method comprising administering the pharmaceutical composition as a monotherapy or in combination with a secondary agent such as immune checkpoint inhibitors, DNA damaging agents, or ionising radiation.

In embodiments, the pharmaceutical composition is for use in a method comprising administering the pharmaceutical composition in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the pharmaceutical composition simultaneously, sequentially or separately from the one or more additional therapeutic agent.

In embodiments, the pharmaceutical composition is for use in a method comprising administering the pharmaceutical composition in combination with one or more therapy selected from: radiation therapy, chemotherapy, immunotherapy and targeted therapy.

In another aspect of this disclosure, there is provided a pharmaceutical composition comprising a compound of formula (4a) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier.

In another aspect of this disclosure, there is provided a pharmaceutical composition comprising a compound of formula (4a) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier, for use in medicine.

In embodiments, the pharmaceutical composition is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the pharmaceutical composition is for use in the treatment or prophylaxis of fibrotic disease or inflammatory disease.

In embodiments, the pharmaceutical composition is for use in a method comprising administering the compound orally, topically, by inhalation, by intranasal administration, by intracerebroventricular; or systemically by intravenous, intraperitoneal, subcutaneous, or intramuscular injection.

In embodiments, the pharmaceutical composition is for use in a method comprising administering the pharmaceutical composition as a monotherapy or in combination with a secondary agent such as immune checkpoint inhibitors, DNA damaging agents, or ionising radiation.

In embodiments, the pharmaceutical composition is for use in a method comprising administering the pharmaceutical composition in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the pharmaceutical composition simultaneously, sequentially or separately from the one or more additional therapeutic agent.

In embodiments, the pharmaceutical composition is for use in a method comprising administering the pharmaceutical composition in combination with one or more therapy selected from: radiation therapy, chemotherapy, immunotherapy and targeted therapy.

In another aspect of this disclosure, there is provided a pharmaceutical composition comprising a compound of formula (4b) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier.

In another aspect of this disclosure, there is provided a pharmaceutical composition comprising a compound of formula (4b) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier, for use in medicine.

In embodiments, the pharmaceutical composition is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the pharmaceutical composition is for use in the treatment or prophylaxis of fibrotic disease or inflammatory disease.

In embodiments, the pharmaceutical composition is for use in a method comprising administering the compound orally, topically, by inhalation, by intranasal administration, by intracerebroventricular; or systemically by intravenous, intraperitoneal, subcutaneous, or intramuscular injection.

In embodiments, the pharmaceutical composition is for use in a method comprising administering the pharmaceutical composition as a monotherapy or in combination with a secondary agent such as immune checkpoint inhibitors, DNA damaging agents, or ionising radiation.

In embodiments, the pharmaceutical composition is for use in a method comprising administering the pharmaceutical composition in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the pharmaceutical composition simultaneously, sequentially or separately from the one or more additional therapeutic agent.

In embodiments, the pharmaceutical composition is for use in a method comprising administering the pharmaceutical composition in combination with one or more therapy selected from: radiation therapy, chemotherapy, immunotherapy and targeted therapy.

In another aspect of this disclosure, there is provided a pharmaceutical composition comprising a compound of formula (4c) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier.

In another aspect of this disclosure, there is provided a pharmaceutical composition comprising a compound of formula (4c) as described above, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier, for use in medicine.

In embodiments, the pharmaceutical composition is for use in the treatment or prophylaxis of a cancer or proliferative disease or disorder, for example wherein the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma.

In embodiments, the pharmaceutical composition is for use in the treatment or prophylaxis of fibrotic disease or inflammatory disease.

In embodiments, the pharmaceutical composition is for use in a method comprising administering the compound orally, topically, by inhalation, by intranasal administration, by intracerebroventricular; or systemically by intravenous, intraperitoneal, subcutaneous, or intramuscular injection.

In embodiments, the pharmaceutical composition is for use in a method comprising administering the pharmaceutical composition as a monotherapy or in combination with a secondary agent such as immune checkpoint inhibitors, DNA damaging agents, or ionising radiation.

In embodiments, the pharmaceutical composition is for use in a method comprising administering the pharmaceutical composition in combination with one or more additional therapeutic agent(s). In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the administering comprises administering the pharmaceutical composition simultaneously, sequentially or separately from the one or more additional therapeutic agent.

In embodiments, the pharmaceutical composition is for use in a method comprising administering the pharmaceutical composition in combination with one or more therapy selected from: radiation therapy, chemotherapy, immunotherapy and targeted therapy.

In another aspect of this disclosure, there is provided a method of treatment comprising administration of a therapeutically effective amount of a compound of formula (1), formula (2), formula (2a), formula (2aa), formula (2ab), formula (2ac), formula (2ad), formula (2b), formula (3), formula (4), formula (4a), formula (4b), formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, to a patient in need thereof.

In embodiments, the patient has a cancer or proliferative disease or disorder. In embodiments, the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from colon cancer, medulloblastoma, head and neck cancer, myelodysplastic syndrome, pancreatic cancer, prostate cancer and melanoma.

In embodiments, the patient has fibrotic disease or inflammatory disease.

In embodiments, the method comprises administering the compound orally, topically, by inhalation, by intranasal administration, by intracerebroventricular; or systemically by intravenous, intraperitoneal, subcutaneous, or intramuscular injection.

In embodiments, the method comprises administering the compound of formula (1), formula (2), formula (2a), formula (2aa), formula (2ab), formula (2ac), formula (2ad), formula (2b), formula (3), formula (4), formula (4a), formula (4b), formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, as a monotherapy or in combination with a secondary agent such as immune checkpoint inhibitors, DNA damaging agents, or ionising radiation.

In embodiments, the method comprises administering the compound of formula (1), formula (2), formula (2a), formula (2aa), formula (2ab), formula (2ac), formula (2ad), formula (2b), formula (3), formula (4), formula (4a), formula (4b), formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more additional therapeutic agent. In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor.

In embodiments, the method comprises administering the compound of formula (1), formula (2), formula (2a), formula (2aa), formula (2ab), formula (2ac), formula (2ad), formula (2b), formula (3), formula (4), formula (4a), formula (4b), formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, simultaneously, sequentially or separately from the one or more additional therapeutic agent.

In embodiments, the method comprising administering the compound of formula (1), formula (2), formula (2a), formula (2aa), formula (2ab), formula (2ac), formula (2ad), formula (2b), formula (3), formula (4), formula (4a), formula (4b), formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more therapy selected from: radiation therapy, chemotherapy, immunotherapy and targeted therapy.

In another aspect of this disclosure, there is provided a method of treating a cancer or proliferative disease or disorder, comprising administering one or more compound(s) of formula (1), formula (2), formula (2a), formula (2aa), formula (2ab), formula (2ad), formula (2ac), formula (2b), formula (3), formula (4), formula (4a), formula (4b), formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, to a subject in need thereof. In embodiments, the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from colon cancer, medulloblastoma, head and neck cancer, myelodysplastic syndrome, pancreatic cancer, prostate cancer and melanoma. In embodiments, the method comprises administering the compound(s) of formula (1), formula (2), formula (2a), formula (2aa), formula (2ab), formula (2ac), formula (2ad), formula (2b), formula (3), formula (4), formula (4a), formula (4b), formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, orally, topically, by inhalation, by intranasal administration, by intracerebroventricular; or systemically by intravenous, intraperitoneal, subcutaneous, or intramuscular injection.

In another aspect of this disclosure, there is provided a method of treating fibrotic disease or inflammatory disease, comprising administering one or more compound(s) of formula (1), formula (2), formula (2a), formula (2aa), formula (2ab), formula (2ad), formula (2ac), formula (2b), formula (3), formula (4), formula (4a), formula (4b), formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, to a subject in need thereof. In embodiments, the method comprises administering the compound(s) of formula (1), formula (2), formula (2a), formula (2aa), formula (2ab), formula (2ac), formula (2ad), formula (2b), formula (3), formula (4), formula (4a), formula (4b), formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, orally, topically, by inhalation, by intranasal administration, by intracerebroventricular; or systemically by intravenous, intraperitoneal, subcutaneous, or intramuscular injection.

In embodiments, the method comprises administering the compound of formula (1), formula (2), formula (2a), formula (2aa), formula (2ab), formula (2ac), formula (2ad), formula (2b), formula (3), formula (4), formula (4a), formula (4b), formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, as a monotherapy or in combination with a secondary agent such as immune checkpoint inhibitors, DNA damaging agents, or ionising radiation.

In embodiments, the method comprises administering the compound(s) of formula (1), formula (2), formula (2a), formula (2aa), formula (2ab), formula (2ac), formula (2ad), formula (2b), formula (3), formula (4), formula (4a), formula (4b), formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more additional therapeutic agent. In embodiments, the one or more additional therapeutic agent is selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor.

In embodiments, the method comprises administering the of formula (1), formula (2), formula (2a), formula (2aa), formula (2ab), formula (2ac), formula (2ad), formula (2b), formula (3), formula (4), formula (4a), formula (4b), formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, simultaneously, sequentially or separately from the one or more additional therapeutic agent.

In embodiments, the method comprising administering the compound(s) of formula (1), formula (2), formula (2a), formula (2aa), formula (2ab), formula (2ac), formula (2ad), formula (2b), formula (3), formula (4), formula (4a), formula (4b), formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, in combination with one or more therapy selected from: radiation therapy, chemotherapy, immunotherapy and targeted therapy.

In another aspect of this disclosure, there is provided the use of a compound of formula (1), formula (2), formula (2a), formula (2aa), formula (2ab), formula (2ac), formula (2ad), formula (2b), formula (3), formula (4), formula (4a), formula (4b), formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, for the manufacture of a medicament.

In embodiments, the use of the compound of formula (1), formula (2), formula (2a), formula (2aa), formula (2ab), formula (2ac), formula (2ad), formula (2b), formula (3), formula (4), formula (4a), formula (4b), formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for the manufacture of a medicament for the treatment or prophylaxis of a cancer or proliferative disease or disorder. In embodiments, the cancer or proliferative disease or disorder is selected from: adrenal gland cancer, anal cancer, angiosarcoma, bladder cancer, blastic plasmacytoid dendritic cell neoplasm, bone cancer, brain cancer, breast cancer, bronchogenic carcinoma, central nervous system (CNS) cancer, cervical cancer, chondrosarcoma, colon cancer, colorectal cancer, cancer of connective tissue, esophageal cancer, embryonal carcinoma, fibrosarcoma, glioblastomas, medulloblastoma, head and neck cancer, hematological cancer, kidney cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), myelodysplastic syndrome, liposarcoma, liver cancer, lung cancer, lymphoid cancers (e.g., Hodgkin's and non-Hodgkin's lymphomas), mesothelioma, multiple myeloma, muscular cancer, myxosarcoma, neuroblastomas, ocular cancer, oral/digestive tract cancer, osteogenic sarcoma, ovarian cancer, papillary carcinoma, pancreatic cancer, polycythemia vera, prostate cancer, renal cancer, retinal cancer, skin cancer, small cell lung carcinoma, stomach cancer, testicular cancer, throat cancer, thyroid cancer, uterine cancer, vaginal cancer, vulvar cancer, gliomas, melanoma, non-small cell lung cancer and acute myeloid leukemia (AML). In embodiments, the cancer or proliferative disease or disorder is selected from: colon cancer, medulloblastoma, head and neck cancer, myelodysplastic syndrome, pancreatic cancer, prostate cancer and melanoma. In embodiments, the treatment comprises administering the medicament orally, topically, by inhalation, by intranasal administration, by intracerebroventricular; or systemically by intravenous, intraperitoneal, subcutaneous, or intramuscular injection.

In embodiments, the use of the compound of formula (1), formula (2), formula (2a), formula (2aa), formula (2ab), formula (2ac), formula (2ad), formula (2b), formula (3), formula (4), formula (4a), formula (4b), formula (4c), or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, is for the manufacture of a medicament for the treatment or prophylaxis of fibrotic disease or inflammatory disease. In embodiments, the treatment comprises administering the medicament orally, topically, by inhalation, by intranasal administration, by intracerebroventricular; or systemically by intravenous, intraperitoneal, subcutaneous, or intramuscular injection.

In embodiments, the treatment comprises administering the medicament as a monotherapy or in combination with a secondary agent such as immune checkpoint inhibitors, DNA damaging agents, or ionising radiation.

In embodiments, the treatment comprises administering the medicament in combination with one or more additional therapeutic agent(s), for example one or more additional therapeutic agents selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor. In embodiments, the treatment comprises administering the medicament, simultaneously, sequentially or separately from the one or more additional therapeutic agent. In embodiments, the medicament comprises one or more additional therapeutic agent(s), for example one or more additional therapeutic agents selected from an EGFR inhibitor, CDK4/6 inhibitor, CDK2 inhibitor, USP1 inhibitor, ATR inhibitor, WEE1 inhibitor, Polθ inhibitor, PKMYT1 inhibitor, ATM inhibitor, DNA-PK, inhibitor, CHK1 inhibitor, PRMT5 inhibitor, anti-androgen therapy (in prostate cancer) and anti-angiogenesis inhibitor.

In embodiments, the treatment comprises administering the medicament in combination with one or more therapy selected from: radiation therapy, chemotherapy, immunotherapy and targeted therapy.

### Dosage Forms, Medicaments and Pharmaceuticals

The compounds, molecules or agents of the invention may be used to treat (e.g. cure, alleviate or prevent) one or more diseases, infections or disorders. Thus, in accordance with the invention, the compounds and molecules may be manufactured into medicaments or may be incorporated or formulated into pharmaceutical compositions.

The molecules, compounds and compositions of the invention may be administered by any convenient route, for example, methods of administration include intradermal, intramuscular, intraperitoneal, intravenous, subcutaneous, intranasal, epidural, oral, sublingual, intranasal, intravaginal, transdermal, rectally, by inhalation, or topically to the skin. Delivery systems are also known to include, for example, encapsulation in liposomes, microgels, microparticles, microcapsules, capsules, etc. Any other suitable delivery system known in the art is also envisioned in use of the present invention.

Administration can be systemic or local. The mode of administration may be left to the discretion of the practitioner.

The dosage administered will, of course, vary depending upon known factors, such as the pharmacodynamic properties of the particular active agent; the chosen mode and route of administration; the age, health and weight of the recipient; the nature of the disease or disorder to be treated; the extent of the symptoms; any simultaneous or concurrent treatments; the frequency of treatment; and the effect desired. In general, a daily dosage of active agent of between about 0.001 and about 1,000 mg/kg of body weight can be expected.

For some applications, the dosage may suitably be within the range of about 0.01 to about 100 mg/kg, between about 0.1 to about 25 mg/kg, or between about 0.1 and 10 mg/kg.

Depending on known factors, such as those noted above, the required dosage of the active agent of the invention may be administered in a single daily dose, or the total daily dosage may be administered in divided doses of two, three, or four times daily.

Dosage forms of the pharmaceutical compositions of the invention suitable for administration may contain from about 1 mg to about 2,000 mg of the active ingredient per unit.

The “effective amount’ or ‘therapeutically effective amount’ is meant to describe an amount of compound or a composition of the disclosure that is effective in curing, inhibiting, alleviating, reducing or preventing the adverse effects of the diseases or disorders to be treated, or the amount necessary to achieve a physiological or biochemically-detectable effect. Thus, at the effective amount, the compound or agent is able to produce the desired therapeutic, ameliorative, inhibitory or preventative effect in relation to disease or disorder.

Beneficially, an effective amount of the compound or composition of the disclosure may have the effect of inhibiting GPR68. Diseases or disorders which may benefit from GPR68 inhibition include, for example, proliferative diseases or disorders and cancer.

When administered to a subject, a compound of the invention is suitably administered as a component of a composition that comprises a pharmaceutically acceptable carrier or vehicle.

One or more additional pharmaceutical acceptable carrier (such as diluents, adjuvants, excipients or vehicles) may be combined with the compound of the invention in a pharmaceutical composition. Suitable pharmaceutical carriers are described in “Remington's Pharmaceutical Science” by E. W. Martin. Pharmaceutical formulations and compositions of the invention are formulated to conform to regulatory standards and according to the chosen route of administration.

Acceptable pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilising, thickening, lubricating and colouring agents may be used. When administered to a subject, the pharmaceutically acceptable vehicles are generally sterile. Water is a suitable vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or buffering agents.

The medicaments and pharmaceutical compositions of the invention can take the form of solutions, suspensions, emulsion, tablets, pills, pellets, powders, gels, capsules (for example, capsules containing liquids or powders), modified-release formulations (such as slow or sustained-release formulations), suppositories, emulsions, aerosols, sprays, suspensions, or any other form suitable for use. Other examples of suitable pharmaceutical vehicles are described in Remingtons Pharmaceutical Sciences, Alfonso R. Gennaro ed., Mack Publishing Co. Easton, Pa., 19^{th} ed., 1995, see for example pages 1447-1676.

Suitably, the therapeutic compositions or medicaments of the invention are formulated in accordance with routine procedures as a pharmaceutical composition adapted for oral administration (more suitably for humans). Compositions for oral delivery may be in the form of tablets, lozenges, aqueous or oily suspensions, granules, powders, emulsions, capsules, syrups, or elixirs, for example. Thus, in one embodiment, the pharmaceutically acceptable vehicle is a capsule, tablet or pill.

Orally administered compositions may contain one or more agents, for example, sweetening agents such as fructose, aspartame or saccharin; flavouring agents such as peppermint, oil of wintergreen, or cherry; colouring agents; and preserving agents, to provide a pharmaceutically palatable preparation. When the composition is in the form of a tablet or pill, the compositions may be coated to delay disintegration and absorption in the gastrointestinal tract, so as to provide a sustained release of active agent over an extended period of time. Selectively permeable membranes surrounding an osmotically active driving compound are also suitable for orally administered compositions. In these dosage forms, fluid from the environment surrounding the capsule is imbibed by the driving compound, which swells to displace the agent or agent composition through an aperture. These dosage forms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade. For oral formulations, the location of release may be the stomach, the small intestine (the duodenum, the jejunem, or the ileum), or the large intestine. One skilled in the art is able to prepare formulations that will not dissolve in the stomach, yet will release the material in the duodenum or elsewhere in the intestine. Suitably, the release will avoid the deleterious effects of the stomach environment, either by protection of the peptide (or derivative) or by release of the peptide (or derivative) beyond the stomach environment, such as in the intestine. To ensure full gastric resistance a coating impermeable to at least pH 5.0 would be essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac, which may be used as mixed films.

To aid dissolution of the therapeutic agent into the aqueous environment a surfactant might be added as a wetting agent. Surfactants may include anionic detergents such as sodium lauryl sulfate, dioctyl sodium sulfosuccinate and dioctyl sodium sulfonate. Cationic detergents might be used and could include benzalkonium chloride or benzethomium chloride. Potential nonionic detergents that could be included in the formulation as surfactants include: lauromacrogol 400, polyoxyl 40 stearate, polyoxyethylene hydrogenated castor oil 10, 50 and 60, glycerol monostearate, polysorbate 20, 40, 60, 65 and 80, sucrose fatty acid ester, methyl cellulose and carboxymethyl cellulose. These surfactants, when used, could be present in the formulation of the compound or derivative either alone or as a mixture in different ratios.

Typically, compositions for intravenous administration comprise sterile isotonic aqueous buffer. Where necessary, the compositions may also include a solubilising agent.

Another suitable route of administration for the therapeutic compositions of the invention is via pulmonary or nasal delivery.

Additives may be included to enhance cellular uptake of the therapeutic agent of the invention, such as the fatty acids oleic acid, linoleic acid and linolenic acid.

The therapeutic agents of the invention may also be formulated into compositions for topical application to the skin of a subject.

Where the invention provides more than one active agent for use in combination, generally, the agents may be formulated separately or in a single dosage form, depending on the prescribed most suitable administration regime for each of the agents concerned. When the therapeutic agents are formulated separately, the pharmaceutical compositions of the invention may be used in a treatment regime involving simultaneous, separate or sequential administration with the other one or more therapeutic agent. The other therapeutic agent(s) may comprise a compound of the invention or a therapeutic agent known in the art).

The molecules and pharmaceutical compositions of the invention may be formulated and suitable for administration to the central nervous system (CNS) and/or for crossing the blood-brain barrier (BBB).

The invention will now be described by way of the following non-limiting examples.

### EXAMPLES

### General Experimental

### LCMS Methods

### Method 1

Samples were analysed by reverse phase HPLC-MS using an Agilent 1200 Series HPLC system and a 6140 Single quadrupole Mass spectrometer equipped with a multi-mode (APCI+ESI) source.

LCMS was carried out using a Phenomenex Luna® C18 (2)-HST column (2.5 μm, 50×2.0 mm) at 254 nm and 220 nm. The column oven temperature was set to 45° C. The column flow rate was set to 1.0 mL/min and the solvents used were 0.1% formic acid in water (A) and 0.1% formic acid in acetonitrile (B). The method timetable was 0.00-0.50 min (1% B), 0.50-2.00 min (linear gradient from 1% to 100% B), and 2.00-3.25 min (100% B).

### Method 2

Samples were analysed by reverse phase HPLC-MS using an Agilent 1200 Series HPLC system and a 6140 Single quadrupole Mass spectrometer equipped with a multi-mode (APCI+ESI) source.

LCMS was carried out using a Phenomenex Luna® C18 (2)-HST column (2.5 μm, 50×2.0 mm) at 254 nm and 220 nm. The column oven temperature was set to 45° C. The column flow rate was set to 1.0 mL/min and the solvents used were 0.1% formic acid in water (A) and 0.1% formic acid in acetonitrile (B). The method timetable was 0.00-0.50 min (1% B), 0.50-3.50 min (linear gradient from 1% to 100% B), and 3.50-4.25 min (100% B).

### Method 3

Samples were analysed by reverse phase HPLC-MS using an Agilent 1200 Series HPLC system and a 6140 Single quadrupole Mass spectrometer equipped with a multi-mode (APCI+ESI) source.

LCMS was carried out using a Phenomenex Luna® C18 (2)-HST column (2.5 μm, 50×2.0 mm) at 254 nm and 220 nm. The column oven temperature was set to 45° C. The column flow rate was set to 1.0 mL/min and the solvents used were 0.1% formic acid in water (A) and 0.1% formic acid in acetonitrile (B). The method timetable was 0.00-1.50 min (linear gradient from 1% to 100% B), and 1.50-1.90 min (100% B).

### Method 4

Samples were analysed by reverse phase HPLC-MS using an Agilent 1290 Series HPLC system and a 6140 Single quadrupole Mass spectrometer equipped with a multi-mode (APCI+ESI) source.

LCMS was carried out using a Phenomenex Kinetex® C18 100 Å (1.7 μm, 50×2.1 mm) at 254 nm and 220 nm. The column oven temperature was set to 40° C. The column flow rate was set to 0.8 mL/min and the solvents used were 0.1% formic acid in water (A) and 0.1% formic acid in acetonitrile (B). The method timetable was 0.00-0.70 min (5% B), 0.70-3.70 min (linear gradient from 5% to 100% B), and 3.70-4.00 min (100% B).

### Method 5

Samples were analysed by reverse phase HPLC-MS using an Agilent 1290 Series HPLC system and a 6140 Single quadrupole Mass spectrometer equipped with a multi-mode (APCI+ESI) source.

LCMS was carried out using a Phenomenex Luna® C18 (2)-HST column (2.5 μm, 50×2.0 mm) at 254 nm and 220 nm. The column oven temperature was set to 40° C. The column flow rate was set to 1.0 mL/min and the solvents used were 0.1% formic acid in water (A) and 0.1% formic acid in acetonitrile (B). The method timetable was 0.00-1.50 min (linear gradient from 5% to 100% B), and 1.50-1.90 min (100% B).

### Method 6

Samples were analysed by reverse phase HPLC-MS using an Agilent 1200 Series HPLC system and a 6140 Single quadrupole Mass spectrometer equipped with a multi-mode (APCI+ESI) source.

LCMS was carried out using a Phenomenex Luna® C18 (2)-HST column (2.5 μm, 50×2.0 mm) at 254 nm and 220 nm. The column oven temperature was set to 45° C. The column flow rate was set to 1.0 mL/min and the solvents used were 0.1% formic acid in water (A) and 0.1% formic acid in acetonitrile (B). The method timetable was 0.00-0.50 min (1% B), 0.50-2.50 min (linear gradient from 1% to 100% B), and 2.50-3.00 min (100% B).

### Method 7

Samples were analysed by reverse phase HPLC-MS using an Agilent 1200 Series HPLC system and a 6140 Single quadrupole Mass spectrometer equipped with a multi-mode (APCI+ESI) source.

LCMS was carried out using a Phenomenex Luna® C18 (2)-HST column (2.5 μm, 50×2.0 mm) at 254 nm and 220 nm. The column oven temperature was set to 45° C. The column flow rate was set to 1.0 mL/min and the solvents used were 0.1% formic acid in water (A) and 0.1% formic acid in acetonitrile (B). The method timetable was 0.00-0.50 min (1% B), 0.50-4.25 min (linear gradient from 1% to 100% B), and 4.25-5.00 min (100% B).

### Preparative HPLC Methods

### Basic Method

Preparative HPLC was carried out on a Teledyne ACCQPrep HP150 Prep HPLC System with 200-400 nm UV variable wavelength detector, ACCQPrep HP150 AS 2×2-AutoSampler using a Waters xBridge BEH C18 OBD Prep column, 5 UM 19 mm×50 mm i.d. column and a flow rate of 24 mL/minute. Compounds were eluted with a water:MeCN mixture containing 0.1% NH₄OH over 10 minutes using UV detection at 254 nm. Gradient information: 0.0-1.0 min, 10% MeCN; 1.0-9.0 min, ramped from 10% MeCN to 100% MeCN; 9.0-10.0 min, held at 100% MeCN.

### Acidic Method

Preparative HPLC was carried out on a Teledyne ACCQPrep HP150 Prep HPLC System with 200-400 nm UV variable wavelength detector, ACCQPrep HP150 AS 2×2-AutoSampler using a Waters xBridge BEH C18 OBD Prep column, 5 μM 19 mm×50 mm i.d. column and a flow rate of 24 mL/minute. Compounds were eluted with a water:MeCN mixture containing 0.1% formic acid over 10 minutes using UV detection at 254 nm. Gradient information: 0.0-1.0 min, 10% MeCN; 1.0-9.0 min, ramped from 10% MeCN to 100% MeCN; 9.0-10.0 min, held at 100% MeCN.

### Supercritical Fluid Chromatography

SFC separations were carried out using a Waters system comprising of a fluid delivery model, thermocube (set to 4° C.), autosampler, column oven (set to 40° C.), 2489 UV/visible detector (254 nm), fraction collection module, heat exchanger, and back pressure regulator. Semi-preparative separations were achieved using Chiralpak (AS-H or AD-H) or Chiralcel (OD-H or OJ-H) columns (all 10,250 mm, 5 μm). Liquid CO₂ was used as the bulk mobile phase.

### H-cube

Hydrogenations were carried out using a THALESNano H-cube™ HC2-55 or a THALESNano H-cube Mini Plus™ hydrogenation reactor with CatCart® cartridges.

### Flow Reactor

Reactions were carried out using a Uniqsis FlowSyn™ 2011 continuous flow reactor.

### Microwave Reactor

Reactions were carried out using a Biotage® Initiator Classic microwave reactor.

### Column Chromatography

Column chromatography purifications were carried out on either a Biotage Isolera™ or a Teledyne ISCO CombiFlash® NextGen 300+ using Biotage® Sfär or Teledyne ISCO RediSep® silica gel flash columns. Fraction collection was based on UV detection.

### SCX Chromatography

SCX purifications were carried out using Biotage Isolute® SCX-2 cartridges.

After loading the sample in an appropriate solvent, the cartridge was washed with MeOH before elution of the desired compounds with 2 M NH3 in MeOH.

### Abbreviations

AcOH Acetic acid
Aq. Aqueous
BF₃·OEt₂ Boron trifluoride etherate
t-BuOH tertiary butanol
CuTC Copper (I) thiophen-2-carboxylate
DABCO 1,4-Diazabicyclo[2.2.2]octane
d Days
DCC N,N′-Dicyclohexylcarbodiimide
DCM Dichloromethane
DIAD Diisopropyl azodicarboxylate
DIPEA N,N-Diisopropylethylamine
DMAP N,N-Dimethylpyridin-4-amine
DME Dimethyl ether
DMF N,N-Dimethylformamide
DMSO Dimethylsulfoxide
DPPA Diphenyl phosphoryl azide
dppf 1,1′-Bis(diphenylphosphino) ferrocene
EDC/EDCl 1-Ethyl-3-(3-dimethylaminopropyl) carbodiimide
Et₃N Triethylamine
EtOAc Ethyl acetate
(EtO)₃CH triethylorthoformate
EtOH Ethanol
h Hours
HATU O-(7-Azabenzotriazol-1-yl)-N,N,N′,N′-tetramethyluronium hexafluorophosphate
HF·pyr HF-pyridine
HOBt 1-Hydroxybenzotriazole
HPLC High Performance Liquid Chromatography
IPA Isopropyl alcohol
LCMS Liquid Chromatography Mass Spectrometry
LiHMDS Lithium hexamethyldisilazide
MeCN Acetonitrile
MeOH Methanol
min Minutes
NBS N-Bromosuccinimide
NCS N-Chlorosuccinimide
NMP N-Methyl-2-pyrrolidone
NMR Nuclear Magnetic Resonance
Pd(OAc)₂ Palladium (II) acetate
i-PrOH Isopropyl alcohol
PS-PPh₃ Polymer-supported triphenylphosphine
RaNi Raney nickel
RT Room temperature
sat. saturated
SCX Strong Cation Exchange
SFC Supercritical Fluid Chromatography
STAB Sodium triacetoxyborohydride
T3P 1-Propanephosphonic anhydride
TBDMSCl tert-Butyldimethylsilyl chloride
TBME tert-Butyl methyl ether
TFA Trifluoroacetic acid
THF Tetrahydrofuran

### Example 1—Compounds

### Synthesis of 4-methylindan-2-amine (General Method A)

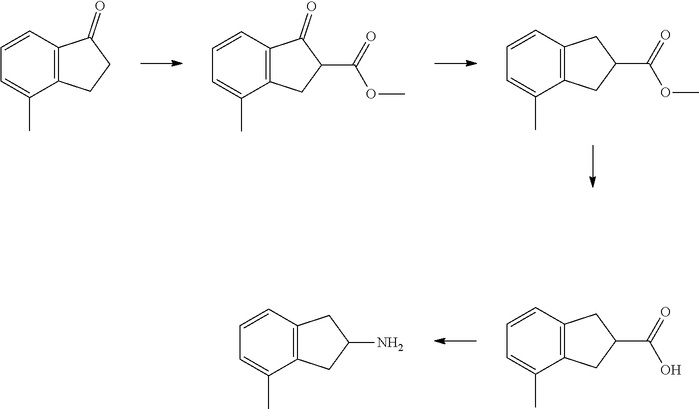

### Synthesis of methyl 4-methyl-1-oxo-indane-2-carboxylate

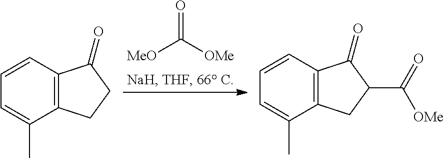

Dimethyl carbonate (430 μL, 5.13 mmol) and NaH (273 mg, 6.84 mmol) were added sequentially to a solution of 4-methylindan-1-one (500 mg, 3.42 mmol) in THF (10 mL) at room temperature. The reaction was heated at 66° C. for 30 min, then cooled to room temperature and acidified with 5 M aq. HCl. The reaction mixture was diluted with EtOAc and the layers were separated. The aqueous layer was extracted with EtOAc (×2), and the combined organic layers were washed with water and brine, dried over Na₂SO₄, and concentrated in vacuo. The crude product was purified by column chromatography (eluting with 0 to 50% EtOAc in heptane) to provide the title compound (490 mg).

¹H NMR (400 MHZ, CDCl₃) δ 7.66-7.58 (m, 1H), 7.54-7.40 (m, 1H), 7.32 (t, J=7.5 Hz, 1H), 3.80 (s, 3H), 3.75 (dd, J=8.2, 3.9 Hz, 1H), 3.49-3.40 (m, 1H), 3.32-3.22 (m, 1H), 2.38 (s, 3H)

LCMS (Method 2): 1.61 min, 205.2 [M+H]⁺

### Synthesis of methyl 4-methylindane-2-carboxylate

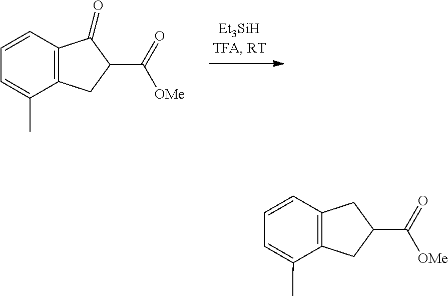

Et₃SiH (1.66 mL, 10.40 mmol) was added to a solution of methyl 4-methyl-1-oxo-indane-2-carboxylate (354 mg, 1.73 mmol) in TFA (6 mL, 78.64 mmol) and the reaction was stirred at room temperature for 96 h. The reaction mixture was concentrated in vacuo and the resulting residue was diluted with DCM and basified with sat. aq. NaHCO₃. The layers were separated, and the organic layer was washed with water, dried through a hydrophobic frit, and concentrated in vacuo. The crude product was purified by column chromatography (eluting with 0 to 50% EtOAc in heptane) to provide the title compound (263 mg).

¹H NMR (400 MHZ, CDCl₃) δ 7.13-7.02 (m, 2H), 7.01-6.94 (m, 1H), 3.74 (s, 3H), 3.41-3.30 (m, 1H), 3.30-3.20 (m, 2H), 3.20-3.09 (m, 2H), 2.26 (s, 3H)

LCMS (Method 2): 2.60 min (product does not ionise)

### Synthesis of 4-methylindane-2-carboxylic Acid

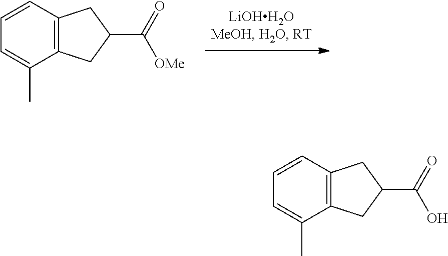

LiOH·H₂O (299 mg, 7.12 mmol) was added to a solution of methyl 4-methylindane-2-carboxylate (271 mg, 1.42 mmol) in MeOH (2 mL) and water (1 mL) and the reaction was stirred at room temperature for 16 h. The reaction mixture was concentrated in vacuo and the resulting residue was dissolved in DCM and acidified with 5 M aq. HCl. The layers were separated, and the organic layer was washed with water and brine, dried over Na₂SO₄, and concentrated in vacuo to provide the title compound (231 mg).

¹H NMR (400 MHZ, CDCl₃) δ 7.14-7.07 (m, 1H), 7.05 (d, J=7.2 Hz, 1H), 7.02-6.96 (m, 1H), 3.46-3.33 (m, 1H), 3.32-3.23 (m, 2H), 3.20 (d, J=8.2 Hz, 2H), 2.26 (s, 3H).

Exchangeable proton not observed.

LCMS (Method 2): 2.16 min (product does not ionise)

### Synthesis of 4-methylindan-2-amine

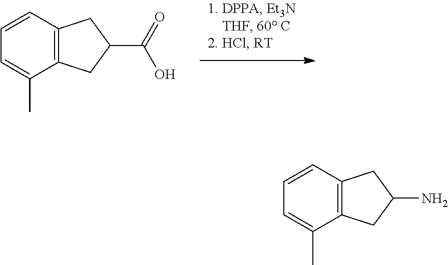

Et₃N (460 μL, 3.28 mmol) and diphenylphosphoryl azide (338 μL, 1.57 mmol) were added sequentially to a solution of 4-methylindane-2-carboxylic acid (231 mg, 1.31 mmol) in THF (4 mL). The reaction was heated at 60° C. for 2 h, then cooled first to room temperature and then to 0° C. in an ice bath. 5 M aq. HCl (1 mL) was added and the reaction was allowed to warm to room temperature and stirred for 16 h. The reaction mixture was diluted with DCM and the layers were separated. The aqueous layer was extracted with DCM, then basified with 2 M aq. NaOH and further extracted with DCM (×3). The combined organic layers were dried over Na₂SO₄ and concentrated in vacuo to provide the title compound (56 mg).

¹H NMR (400 MHZ, CDCl₃) δ 7.15-6.99 (m, 3H), 3.88-3.77 (m, 1H), 3.24-3.19 (m, 2H), 3.18-3.07 (m, 2H), 2.82-2.61 (m, 2H), 2.23 (s, 3H)

LCMS (Method 2): 1.13 min, 148.2 [M+H]⁺

### Synthesis of 4-chloroindan-2-amine

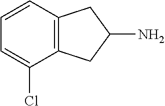 [SMILES: NC1Cc2cccc(Cl)c2C1]

4-Chloroindan-2-amine (611 mg) was synthesised following a similar procedure to that described in General Method A.

¹H NMR (400 MHZ, CDCl₃) δ 7.17-7.11 (m, 1H), 7.11-7.06 (m, 2H), 3.87 (tt, J=6.8, 4.8 Hz, 1H), 3.26 (d, J=6.8 Hz, 1H), 3.22 (d, J=6.8 Hz, 1H), 2.78-2.74 (m, 1H), 2.74-2.69 (m, 1H), 1.46 (br. s, 2H)

LCMS (Method 1): 1.07 min, 168.2, 170.2 [M+H]⁺

### Synthesis of 4-fluoroindan-2-amine

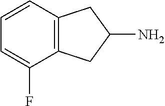 [SMILES: NC1Cc2cccc(F)c2C1]

4-Fluoroindan-2-amine (90 mg) was synthesised following a similar procedure to that described in General Method A.

¹H NMR (400 MHZ, CDCl₃) δ 7.18-7.07 (m, 1H), 6.99 (d, J=7.4 Hz, 1H), 6.89-6.78 (m, 1H), 3.95-3.85 (m, 1H), 3.28-3.16 (m, 2H), 2.73 (dd, J=16.1, 5.0 Hz, 2H). Exchangeable protons not observed.

### Synthesis of 4,6-difluoroindan-2-amine

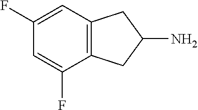 [SMILES: NC1Cc2cc(F)cc(F)c2C1]

4,6-Difluoroindan-2-amine (121 mg) was synthesised following a similar procedure to that described in General Method A.

¹H NMR (400 MHZ, CDCl₃) δ 6.79-6.69 (m, 1H), 6.60 (td, J=9.2, 2.2 Hz, 1H), 3.97-3.82 (m, 1H), 3.23-3.08 (m, 2H), 2.74-2.59 (m, 2H). Exchangeable protons not observed.

### Synthesis of compound 1, N7-indan-2-ylpyrazolo[1,5-a]pyrimidine-3,7-dicarboxamide (General Method B)

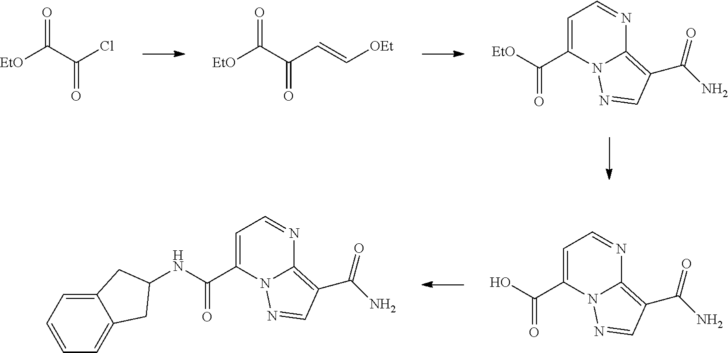 [SMILES: CCOC(=O)C(=O)Cl ; CCO/C=C/C(=O)C(=O)OCC ; CCOC(=O)c1ccnc2c(C(N)=O)cnn12 ; NC(=O)c1cnn2c(C(=O)NC3Cc4ccccc4C3)ccnc12 ; NC(=O)c1cnn2c(C(=O)O)ccnc12]

### Synthesis of ethyl (E)-4-ethoxy-2-oxo-but-3-enoate

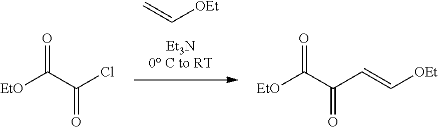

Ethyl 2-chloro-2-oxo-acetate (8.2 mL, 73.20 mmol) was added slowly over 10 min to a solution of ethyl vinyl ether (34.8 mL, 366.22 mmol) and Et₃N (12.25 mL, 87.89 mmol) at 0° C. The reaction was warmed to room temperature and stirred overnight. The reaction mixture was diluted with DCM and washed with sat. aq. NaHCO₃. The organic layer was washed with water, dried through a hydrophobic frit, and concentrated in vacuo to provide the title compound (12.48 g).

¹H NMR (400 MHZ, CDCl₃) δ 7.87 (d, J=12.7 Hz, 1H), 6.18 (d, J=12.7 Hz, 1H), 4.32 (q, J=7.2 Hz, 2H), 4.06 (q, J=7.1 Hz, 2H), 1.47-1.28 (m, 6H)

LCMS (Method 1): 1.59 min, 173.2 [M+H]⁺

### Synthesis of ethyl 3-carbamoylpyrazolo[1,5-a]pyrimidine-7-carboxylate

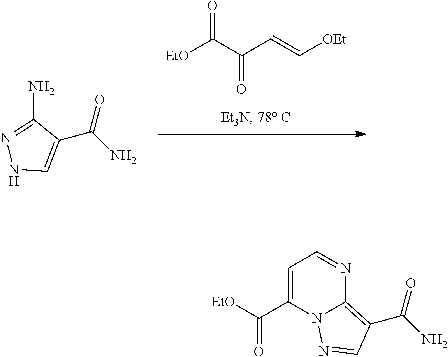 [SMILES: NC(=O)c1c[nH]nc1N ; CCO/C=C/C(=O)C(=O)OCC ; CCOC(=O)c1ccnc2c(C(N)=O)cnn12]

A solution of 5-amino-1H-pyrazole-4-carboxamide (806 mg, 6.39 mmol) and ethyl (E)-4-ethoxy-2-oxo-but-3-enoate (1.1 g, 6.39 mmol) in EtOH (9 mL) was heated at 78° C. for 16 h. The reaction mixture was cooled to room temperature and concentrated in vacuo. The residue was suspended in EtOH and the resulting precipitate was collected by filtration, washed with EtOH, and dried to provide the title compound (895 mg).

¹H NMR (400 MHz, DMSO-d₆) δ 8.90 (d, J=4.3 Hz, 1H), 8.64 (s, 1H), 7.62 (d, J=4.3 Hz, 1H), 7.56 (s, 1H), 7.45 (d, J=17.6 Hz, 1H), 4.48 (q, J=7.1 Hz, 2H), 1.36 (t, J=7.1 Hz, 3H)

LCMS (Method 2): 1.49 min, 235.2 [M+H]⁺

### Synthesis of 3-carbamoylpyrazolo[1,5-a]pyrimidine-7-carboxylic acid

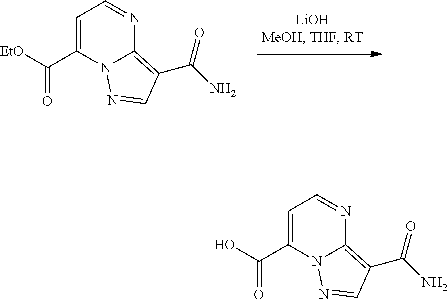 [SMILES: CCOC(=O)c1ccnc2c(C(N)=O)cnn12 ; NC(=O)c1cnn2c(C(=O)O)ccnc12]

4 M aq. LiOH (4.8 mL, 19.11 mmol) was added to a solution of ethyl 3-carbamoylpyrazolo[1,5-a]pyrimidine-7-carboxylate (895 mg, 3.82 mmol) in THF (10 mL) and MeOH (3 mL) and the reaction was stirred at room temperature for 16 h. The reaction mixture was concentrated in vacuo and the aqueous residue was acidified with 5 M aq. HCl. The resulting precipitate was collected by filtration, washed with water, and dried to provide the title compound (710 mg).

¹H NMR (400 MHz, DMSO-d₆) δ 8.89 (d, J=4.3 Hz, 1H), 8.63 (s, 1H), 7.56 (d, J=4.3 Hz, 2H), 7.49 (s, 1H). Additional exchangeable proton not observed.

LCMS (Method 2): 0.74 min, 207.1 [M+H]⁺

### Synthesis of compound 1, N7-indan-2-ylpyrazolo[1,5-a]pyrimidine-3,7-dicarboxamide

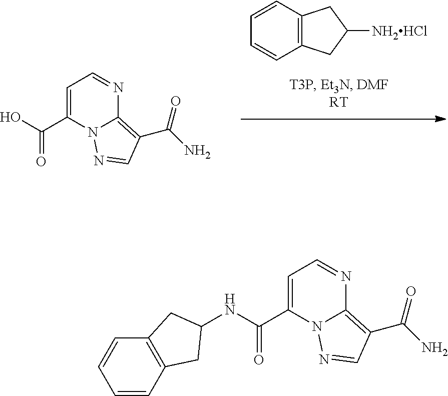

1-Propanephosphonic anhydride (50 wt % in DMF; 1.3 mL, 2.18 mmol) and Et₃N (304 μL, 2.18 mmol) were added to a solution of indan-2-amine hydrochloride (136 mg, 0.80 mmol) and 3-carbamoylpyrazolo[1,5-a]pyrimidine-7-carboxylic acid (150 mg, 0.73 mmol) in DMF (1.5 mL) and the reaction was stirred at room temperature for 24 h then quenched with water. The resulting suspension was collected by filtration, washed with water and heptane, and dried to provide the title compound (199 mg).

¹H NMR (400 MHz, DMSO-d₆) δ 9.92 (d, J=7.0 Hz, 1H), 8.92 (d, J=4.4 Hz, 1H), 8.65 (s, 1H), 7.64 (d, J=4.4 Hz, 1H), 7.58 (s, 1H), 7.52 (s, 1H), 7.28 (dd, J=5.4, 3.3 Hz, 2H), 7.22-7.13 (m, 2H), 4.85-4.72 (m, 1H), 3.38 (dd, J=16.1, 7.6 Hz, 2H), 2.99 (dd, J=16.1, 5.4 Hz, 2H)

LCMS (Method 1): 1.58 min, 322.0 [M+H]₊

### Synthesis of compound 2, N7-(4-methylindan-2-yl)pyrazolo[1,5-a]pyrimidine-3,7-dicarboxamide

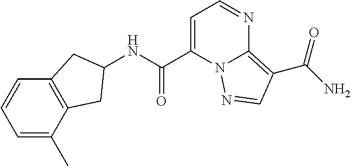 [SMILES: Cc1cccc2c1CC(NC(=O)c1ccnc3c(C(N)=O)cnn13)C2]

N7-(4-methylindan-2-yl)pyrazolo[1,5-a]pyrimidine-3,7-dicarboxamide (15 mg) was synthesised following a similar procedure to that described in General Method B.

1H NMR (400 MHz, DMSO-d₆) δ 9.92 (d, J=7.0 Hz, 1H), 8.92 (d, J=4.3 Hz, 1H), 8.66 (s, 1H), 7.64 (d, J=4.4 Hz, 1H), 7.59 (s, 1H), 7.52 (s, 1H), 7.13-7.04 (m, 2H), 7.04-6.95 (m, 1H), 4.85-4.72 (m, 1H), 3.43-3.33 (m, 2H), 3.04-2.95 (m, 1H), 2.95-2.85 (m, 1H), 2.23 (s, 3H)

LCMS (Method 2): 2.25 min, 336.2 [M+H]⁺

### Synthesis of compound 3, N7-(5-methoxyindan-2-yl)pyrazolo[1,5-a]pyrimidine-3,7-dicarboxamide

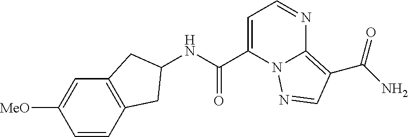 [SMILES: COc1ccc2c(c1)CC(NC(=O)c1ccnc3c(C(N)=O)cnn13)C2]

N7-(5-methoxyindan-2-yl)pyrazolo[1,5-a]pyrimidine-3,7-dicarboxamide (30 mg) was synthesised following a similar procedure to that described in General Method B.

¹H NMR (400 MHz, DMSO-d₆) δ 9.93 (d, J=7.0 Hz, 1H), 8.93 (d, J=4.3 Hz, 1H), 8.66 (s, 1H), 7.65 (d, J=4.3 Hz, 1H), 7.60 (s, 1H), 7.53 (s, 1H), 7.17 (d, J=8.3 Hz, 1H), 6.87 (d, J=2.5 Hz, 1H), 6.75 (dd, J=8.3, 2.5 Hz, 1H), 4.84-4.71 (m, 1H), 3.73 (s, 3H), 3.40-3.25 (m, 2H), 3.00-2.85 (m, 2H)

LCMS (Method 2): 2.11 min, 352.2 [M+H]⁺

### Synthesis of compound 4, N7-(4-chloroindan-2-yl)pyrazolo[1,5-a]pyrimidine-3,7-dicarboxamide

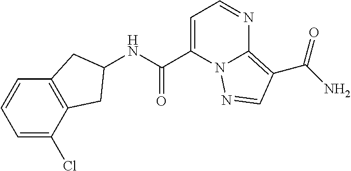 [SMILES: NC(=O)c1cnn2c(C(=O)NC3Cc4cccc(Cl)c4C3)ccnc12]

N7-(4-chloroindan-2-yl)pyrazolo[1,5-a]pyrimidine-3,7-dicarboxamide (45 mg) was synthesised following a similar procedure to that described in General Method B.

¹H NMR (400 MHz, DMSO-d₆) δ 9.93 (d, J=6.9 Hz, 1H), 8.92 (d, J=4.3 Hz, 1H), 8.65 (s, 1H), 7.63 (d, J=4.3 Hz, 1H), 7.59 (br. s, 1H), 7.52 (br. s, 1H), 7.32-7.18 (m, 3H), 4.88-4.75 (m, 1H), 3.47 (dd, J=19.8, 7.7 Hz, 1H), 3.43 (dd, J=19.8, 7.7 Hz, 1H), 3.09 (dd, J=16.6, 5.2 Hz, 1H), 3.03 (dd, J=16.7, 5.1 Hz, 1H)

LCMS (Method 2): 2.26 min, 356.1, 358.2 [M+H]⁺

### Synthesis of compound 5, N7-(1,2,3,4-tetrahydroquinolin-3-yl)pyrazolo[1,5-a]pyrimidine-3,7-dicarboxamide

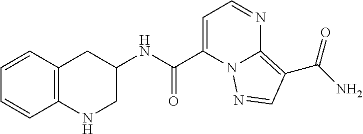 [SMILES: NC(=O)c1cnn2c(C(=O)NC3CNc4ccccc4C3)ccnc12]

N7-(1,2,3,4-tetrahydroquinolin-3-yl)pyrazolo[1,5-a]pyrimidine-3,7-dicarboxamide (18 mg) was synthesised following a similar procedure to that described in General Method B.

¹H NMR (400 MHz, DMSO-d₆) δ 10.02 (d, J=7.3 Hz, 1H), 8.94 (d, J=4.4 Hz, 1H), 8.56 (s, 1H), 7.75 (d, J=4.4 Hz, 1H), 7.60 (s, 1H), 7.54 (s, 1H), 6.94-6.88 (m, 2H), 6.56 (dt, J=7.5, 1.1 Hz, 1H), 6.49 (td, J=7.3, 1.2 Hz, 1H), 5.90 (s, 1H), 4.48-4.38 (m, 1H), 3.47-3.39 (m, 1H), 3.22 (ddd, J=11.6, 6.1, 1.4 Hz, 1H), 3.09 (dd, J=16.0, 4.8 Hz, 1H), 2.84 (dd, J=16.2, 6.1 Hz, 1H)

LCMS (Method 2) 1.88 min, 337.2 [M+H]⁺

### Synthesis of compound 6, N7-chroman-3-ylpyrazolo[1,5-a]pyrimidine-3,7-dicarboxamide

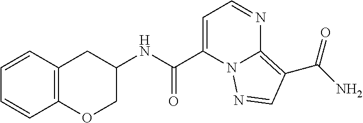 [SMILES: NC(=O)c1cnn2c(C(=O)NC3COc4ccccc4C3)ccnc12]

N7-chroman-3-ylpyrazolo[1,5-a]pyrimidine-3,7-dicarboxamide (17.4 mg) was synthesised following a similar procedure to that described in General Method B.

¹H NMR (400 MHz, DMSO-d₆) δ 10.03 (d, J=5.5 Hz, 1H), 8.93 (d, J=4.3 Hz, 1H), 8.59 (s, 1H), 7.71 (d, J=4.4 Hz, 1H), 7.59 (s, 1H), 7.52 (s, 1H), 7.12 (td, J=7.4, 1.5 Hz, 2H), 6.92-6.82 (m, 2H), 4.54 (s, 1H), 4.31-4.24 (m, 1H), 4.18 (ddd, J=10.9, 5.7, 1.5 Hz, 1H), 3.22 (dd, J=16.6, 5.4 Hz, 1H), 2.92 (dd, J=16.6, 5.6 Hz, 1H)

LCMS (Method 2): 1.984 min, 338.15 [M+H]⁺

### Synthesis of compound 7, N7-(trans-3-fluoro-3-methyl-cyclobutyl)pyrazolo[1,5-a]pyrimidine-3,7-dicarboxamide

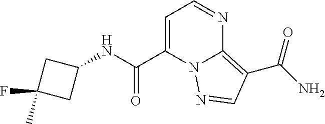 [SMILES: C[C@]1(F)C[C@H](NC(=O)c2ccnc3c(C(N)=O)cnn23)C1]

N7-(trans-3-fluoro-3-methyl-cyclobutyl)pyrazolo[1,5-a]pyrimidine-3,7-dicarboxamide (15 mg) was synthesised following a similar procedure to that described in General Method B.

¹H NMR (400 MHz, DMSO-d₆) δ 9.92 (d, J=6.3 Hz, 1H), 8.93 (d, J=4.4 Hz, 1H), 8.70 (s, 1H), 7.64 (d, J=4.4 Hz, 1H), 7.61 (s, 1H), 7.53 (s, 1H), 4.62-4.50 (m, 1H), 2.77-2.65 (m, 2H), 2.38-2.26 (m, 2H), 1.51 (d, J=22.3 Hz, 3H)

LCMS (Method 2): 1.77 min, 292.2 [M+H]⁺

### Synthesis of compound 8, N7-(cis-3-hydroxy-3-methyl-cyclobutyl)pyrazolo[1,5-a]pyrimidine-3,7-dicarboxamide

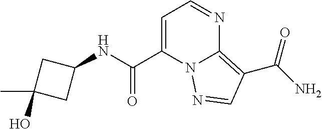 [SMILES: C[C@]1(O)C[C@@H](NC(=O)c2ccnc3c(C(N)=O)cnn23)C1]

N7-(cis-3-hydroxy-3-methyl-cyclobutyl)pyrazolo[1,5-a]pyrimidine-3,7-dicarboxamide (37 mg) was synthesised following a similar procedure to that described in General Method B.

¹H NMR (400 MHz, DMSO-d₆) δ 9.92 (d, J=7.1 Hz, 1H), 8.93 (d, J=4.4 Hz, 1H), 8.72 (s, 1H), 7.67 (d, J=4.4 Hz, 1H), 7.61 (br. s, 1H), 7.55 (br. s, 1H), 5.14 (s, 1H), 4.16-4.02 (m, 1H), 2.47-2.39 (m, 2H), 2.17-2.05 (m, 2H), 1.28 (s, 3H)

LCMS (Method 2): 1.45 min, 290.2 [M+H]⁺

### Synthesis of compound 9, N7-(cis-3-cyanocyclobutyl)pyrazolo[1,5-a]pyrimidine-3,7-dicarboxamide

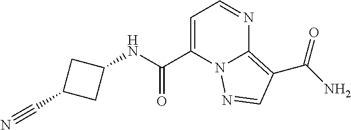 [SMILES: N#C[C@H]1C[C@@H](NC(=O)c2ccnc3c(C(N)=O)cnn23)C1]

N7-(cis-3-cyanocyclobutyl)pyrazolo[1,5-a]pyrimidine-3,7-dicarboxamide (33 mg) was synthesised following a similar procedure to that described in General Method B.

¹H NMR (400 MHz, DMSO-d₆) δ 9.98 (d, J=7.5 Hz, 1H), 8.94 (d, J=4.3 Hz, 1H), 8.71 (s, 1H), 7.64 (d, J=4.4 Hz, 1H), 7.62 (s, 1H), 7.54 (s, 1H), 4.52 (q, J=8.3 Hz, 1H), 3.25-3.11 (m, 1H), 2.82-2.70 (m, 2H), 2.60-2.45 (m, 2H)

LCMS (Method 2): 1.51 min, 285.2 [M+H]⁺

### Synthesis of compound 10, N7-(3,3-diethylcyclobutyl)pyrazolo[1,5-a]pyrimidine-3,7-dicarboxamide

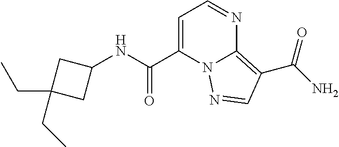 [SMILES: CCC1(CC)CC(NC(=O)c2ccnc3c(C(N)=O)cnn23)C1]

N7-(3,3-diethylcyclobutyl)pyrazolo[1,5-a]pyrimidine-3,7-dicarboxamide (18 mg) was synthesised following a similar procedure to that described in General Method B.

¹H NMR (400 MHz, DMSO-d₆) δ 9.85 (d, J=7.2 Hz, 1H), 8.92 (d, J=4.4 Hz, 1H), 8.70 (s, 1H), 7.64-7.57 (m, 2H), 7.54 (s, 1H), 4.45-4.35 (m, 1H), 2.24-2.15 (m, 2H), 1.82-1.71 (m, 2H), 1.46 (dq, J=24.6, 7.4 Hz, 4H), 0.81 (t, J=7.4 Hz, 3H), 0.74 (t, J=7.4 Hz, 3H)

LCMS (Method 4): 2.28 min, 316.2 [M+H]⁺

### Synthesis of compound 11, N7-(cis-3-methoxycyclobutyl)pyrazolo[1,5-a]pyrimidine-3,7-dicarboxamide

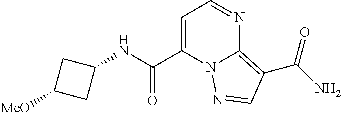 [SMILES: CO[C@H]1C[C@@H](NC(=O)c2ccnc3c(C(N)=O)cnn23)C1]

N7-(cis-3-methoxycyclobutyl)pyrazolo[1,5-a]pyrimidine-3,7-dicarboxamide (88 mg) was synthesised following a similar procedure to that described in General Method B.

1H NMR (400 MHz, DMSO-d₆) δ 9.86 (d, J=7.3 Hz, 1H), 8.92 (d, J=4.3 Hz, 1H), 8.71 (s, 1H), 7.62 (d, J=4.3 Hz, 1H), 7.60 (s, 1H), 7.53 (s, 1H), 4.17-4.03 (m, 1H), 3.75-3.63 (m, 1H), 3.16 (s, 3H), 2.78-2.66 (m, 2H), 1.98-1.86 (m, 2H)

LCMS (Method 2): 1.55 min, 1.57 min, 290.2 [M+H]⁺

### Synthesis of 2-methyl-N-(7-oxaspiro[3.5]nonan-2-yl) propane-2-sulfinamide

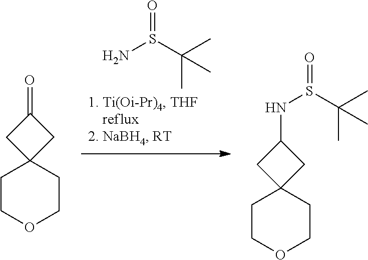

Ti(Oi-Pr)₄ (796 μL, 2.68 mmol) was added to a solution of tert-butylsulfinamide (259 mg, 2.14 mmol) and 7-oxaspiro[3.5]nonan-2-one (250 mg, 1.78 mmol) in THF (10 mL) and the reaction was heated to reflux overnight then cooled to room temperature. NaBH₄ (135 mg, 3.57 mmol) was added and the reaction was stirred at room temperature for 2 h then allowed to stand at room temperature for 5 days. The reaction was quenched with water, filtered, and washed with EtOAc. The filtrate was diluted with EtOAc and water, and the layers were separated. The organic layer was washed with brine, dried over Na₂SO₄, and concentrated in vacuo. The crude product was purified by column chromatography (eluting with 0 to 10% (2 M NH₃ in MeOH) in DCM) to provide the title compound (305 mg).

¹H NMR (400 MHZ, CDCl₃) δ 3.97-3.84 (m, 1H), 3.65-3.59 (m, 2H), 3.58-3.52 (m, 2H), 3.30 (s, 1H), 2.41-2.26 (m, 2H), 1.79-1.68 (m, 2H), 1.60 (dd, J=10.0, 4.8 Hz, 4H), 1.20 (s, 9H)

LCMS (Method 2): 1.87 min, 246.2 [M+H]⁺

### Synthesis of 7-oxaspiro[3.5]nonan-2-amine hydrochloride

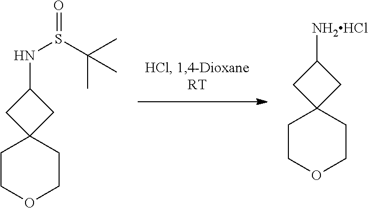

2-Methyl-N-(7-oxaspiro[3.5]nonan-2-yl) propane-2-sulfinamide (305 mg, 1.24 mmol) was suspended in HCl (4 M in 1,4-dioxane; 5 mL, 20.00 mmol) and the reaction was stirred at room temperature for 16 h then concentrated in vacuo. The residue was sonicated in Et₂O, and the resulting precipitate was collected by filtration and dried to provide the title compound (172 mg).

¹H NMR (400 MHz, DMSO-d₆) δ 8.24 (s, 2H), 3.72-3.58 (m, 1H), 3.53-3.42 (m, 4H), 2.19-2.09 (m, 2H), 1.96-1.87 (m, 2H), 1.57-1.48 (m, 4H). Additional exchangeable proton not observed.

### Synthesis of compound 12, N7-(7-oxaspiro[3.5]nonan-2-yl)pyrazolo[1,5-a]pyrimidine-3,7-dicarboxamide

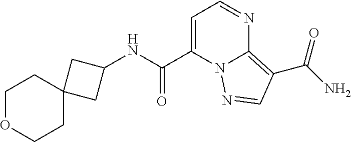 [SMILES: NC(=O)c1cnn2c(C(=O)NC3CC4(CCOCC4)C3)ccnc12]

N7-(7-oxaspiro[3.5]nonan-2-yl)pyrazolo[1,5-a]pyrimidine-3,7-dicarboxamide (22 mg) was synthesised following a similar procedure to that described in General Method B.

¹H NMR (400 MHz, DMSO-d₆) δ 9.88 (d, J=7.1 Hz, 1H), 8.92 (d, J=4.3 Hz, 1H), 8.70 (s, 1H), 7.63 (d, J=4.4 Hz, 1H), 7.60 (s, 1H), 7.54 (s, 1H), 4.54-4.41 (m, 1H), 3.59-3.49 (m, 2H), 3.49-3.41 (m, 2H), 2.39-2.30 (m, 2H), 1.92-1.82 (m, 2H), 1.65-1.58 (m, 2H), 1.58-1.50 (m, 2H)

LCMS (Method 2): 1.73 min, 330.2 [M+H]⁺

### Synthesis of 5,6,7,8-tetrahydroisoquinolin-7-amine

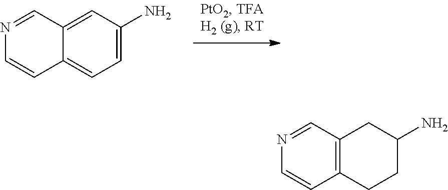 [SMILES: Nc1ccc2ccncc2c1 ; NC1CCc2ccncc2C1]

PtO₂ (47 mg, 0.21 mmol) and isoquinolin-7-amine (300 mg, 2.08 mmol) were dissolved in TFA (5 mL, 65.34 mmol) and the reaction was stirred under a hydrogen atmosphere (balloon) at room temperature for 20 h. The reaction mixture was filtered through a short pad of diatomaceous earth (Dicalite®), washed with EtOAc and MeOH, and concentrated in vacuo. The crude product was purified by SCX chromatography (eluting with 2 M NH₃ in MeOH) and column chromatography (eluting with 0 to 10% (2 M NH₃ in MeOH) in DCM) to provide the title compound (48 mg).

¹H NMR (400 MHz, DMSO-d₆) δ 8.28-8.17 (m, 2H), 7.05 (d, J=5.0 Hz, 1H), 3.07-2.97 (m, 2H), 2.93-2.64 (m, 4H), 2.39 (dd, J=16.4, 9.0 Hz, 1H), 1.91-1.79 (m, 1H), 1.53-1.39 (m, 1H)

### Synthesis of compound 13, N7-(5,6,7,8-tetrahydroisoquinolin-7-yl)pyrazolo[1,5-a]pyrimidine-3,7-dicarboxamide

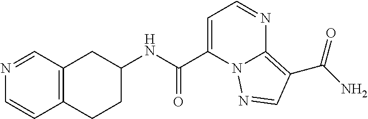 [SMILES: NC(=O)c1cnn2c(C(=O)NC3CCc4ccncc4C3)ccnc12]

N7-(5,6,7,8-tetrahydroisoquinolin-7-yl)pyrazolo[1,5-a]pyrimidine-3,7-dicarboxamide (12 mg) was synthesised following a similar procedure to that described in General Method B.

¹H NMR (400 MHz, DMSO-d₆) δ 9.82 (d, J=7.3 Hz, 1H), 8.94 (d, J=4.3 Hz, 1H), 8.65 (s, 1H), 8.33 (s, 1H), 8.27 (d, J=5.1 Hz, 1H), 7.67 (d, J=4.3 Hz, 1H), 7.60 (s, 1H), 7.53 (s, 1H), 7.14 (d, J=5.1 Hz, 1H), 4.35 (d, J=8.6 Hz, 1H), 3.19 (dd, J=16.7, 5.1 Hz, 1H), 2.93 (t, J=6.5 Hz, 2H), 2.84 (dd, J=16.5, 8.5 Hz, 1H), 2.18-2.07 (m, 1H), 1.92 (s, 1H)

LCMS (Method 2): 1.17 min, 337.2 [M+H]⁺

### Synthesis of N7-(4-bromoindan-2-yl)pyrazolo[1,5-a]pyrimidine-3,7-dicarboxamide

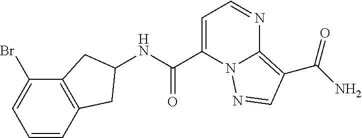 [SMILES: NC(=O)c1cnn2c(C(=O)NC3Cc4cccc(Br)c4C3)ccnc12]

N7-(4-bromoindan-2-yl)pyrazolo[1,5-a]pyrimidine-3,7-dicarboxamide (382 mg) was synthesised following a similar procedure to that described in General Method B.

1H NMR (400 MHz, DMSO-d₆) δ 9.93 (d, J=6.9 Hz, 1H), 8.92 (d, J=4.3 Hz, 1H), 8.66 (s, 1H), 7.63 (d, J=4.3 Hz, 1H), 7.58 (br. s, 1H), 7.52 (br. s, 1H), 7.40 (d, J=7.9 Hz, 1H), 7.30 (d, J=7.4 Hz, 1H), 7.15 (t, J=7.7 Hz, 1H), 4.81 (dtd, J=12.8, 7.5, 5.2 Hz, 1H), 3.51 (dd, J=16.5, 7.7 Hz, 1H), 3.40 (dd, J=16.7, 7.7 Hz, 1H), 3.12 (dd, J=16.5, 5.2 Hz, 1H), 3.00 (dd, J=16.7, 5.1 Hz, 1H)

LCMS (Method 1): 1.68 min, 400.1, 402.1 [M+H]⁺

### Synthesis of compound 14, N7-[4-(3,3,3-trifluoropropyl) indan-2-yl]pyrazolo[1,5-a]pyrimidine-3,7-dicarboxamide

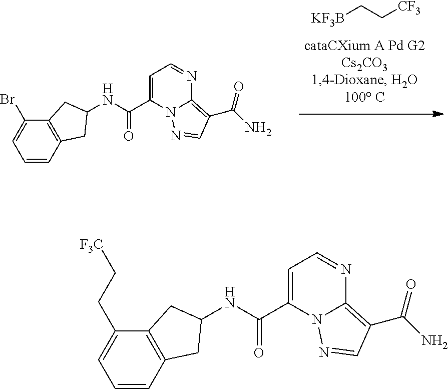

N7-(4-bromoindan-2-yl)pyrazolo[1,5-a]pyrimidine-3,7-dicarboxamide (70 mg, 0.17 mmol), potassium trifluoro (3,3,3-trifluoropropyl) borate (152 mg, 0.74 mmol), cataCXium A Pd G2 (chloro[(di(1-adamantyl)-N-butylphosphine)-2-(2-aminobiphenyl)]palladium (II); 8.4 mg, 0.01 mmol), and Cs₂CO₃ (195 mg, 0.60 mmol) were placed in a vial and the vial was sealed and purged with argon for 5 min. 1,4-Dioxane (2 mL) and water (200 μL) were added, and the mixture was degassed by sparging with a stream of argon for 10 min. The reaction was heated at 100° C. for 16 h then cooled to room temperature. Additional portions of potassium trifluoro (3,3,3-trifluoropropyl) borate (64 mg, 0.31 mmol), cataCXium A Pd G2 (8.4 mg, 0.01 mmol), and Cs₂CO₃ (81 mg, 0.25 mmol) were added and the mixture was degassed by sparging with a stream of argon for 10 min. The reaction was heated at 100° C. for a further 23 h then cooled to room temperature. The reaction mixture was diluted with EtOAc and filtered through a short pad of Dicalite®. The filtrate was concentrated in vacuo and the crude product was purified by column chromatography (eluting with 0 to 5% MeOH in DCM) and preparative HPLC (basic method) to provide the title compound (45 mg).
¹H NMR (400 MHz, DMSO-d₆) δ 9.91 (d, J=6.9 Hz, 1H), 8.93 (d, J=4.3 Hz, 1H), 8.63 (s, 1H), 7.64 (d, J=4.3 Hz, 1H), 7.59 (br. s, 1H), 7.52 (br. s, 1H), 7.20-7.12 (m, 2H), 7.12-7.07 (m, 1H), 4.86-4.73 (m, 1H), 3.39 (ddd, J=16.2, 7.6, 3.8 Hz, 2H), 2.99 (ddd, J=16.1, 8.1, 5.3 Hz, 2H), 2.84-2.74 (m, 2H), 2.62-2.52 (m, 2H)
LCMS (Method 2): 2.44 min, 418.2 [M+H]⁺

### Synthesis of N7-[4-(cyclopenten-1-yl) indan-2-yl]pyrazolo[1,5-a]pyrimidine-3,7-dicarboxamide

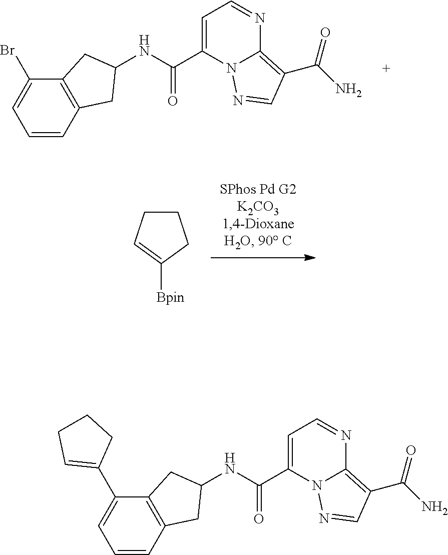

N7-(4-bromoindan-2-yl)pyrazolo[1,5-a]pyrimidine-3,7-dicarboxamide (50 mg, 0.12 mmol), 2-(cyclopenten-1-yl)-4,4,5,5-tetramethyl-1,3,2-dioxaborolane (61 mg, 0.31 mmol), SPhos Pd G2 (chloro(2-dicyclohexylphosphino-2′,6′-dimethoxy-1,1′-biphenyl) [2-(2′-amino-1,1′-biphenyl)]palladium (II); 9 mg, 0.01 mmol), and K₂CO₃ (52 mg, 0.37 mmol) were placed in a vial and the vial was sealed and purged with argon for 5 min. 1,4-Dioxane (2 mL) and water (200 μL) were added and the mixture was sonicated and degassed by sparging with a stream of argon for 5 min. The reaction was heated at 90° C. for 20 h then cooled to room temperature. The reaction mixture was filtered through a short pad of Dicalite® and concentrated in vacuo. The crude product was purified by column chromatography (eluting with 0 to 3% MeOH in DCM) to provide the title compound (45 mg).

¹H NMR (400 MHz, DMSO-d₆) δ 9.91 (d, J=6.9 Hz, 1H), 8.92 (d, J=4.3 Hz, 1H), 8.65 (s, 1H), 7.64 (d, J=4.4 Hz, 1H), 7.58 (br. s, 1H), 7.52 (br. s, 1H), 7.26-7.06 (m, 3H), 6.01 (td, J=2.6, 1.3 Hz, 1H), 4.84-4.71 (m, 1H), 3.54-3.44 (m, 1H), 3.43-3.34 (m, 1H), 3.04 (ddd, J=21.8, 16.3, 5.2 Hz, 2H), 2.76-2.64 (m, 2H), 2.56-2.52 (m, 2H), 2.00-1.85 (m, 2H)

LCMS (Method 2): 2.59 min, 388.2 [M+H]⁺

### Synthesis of compound 15, N7-[5-(2-pyrrolidin-1-ylethyl) indan-2-yl]pyrazolo[1,5-a]pyrimidine-3,7-dicarboxamide

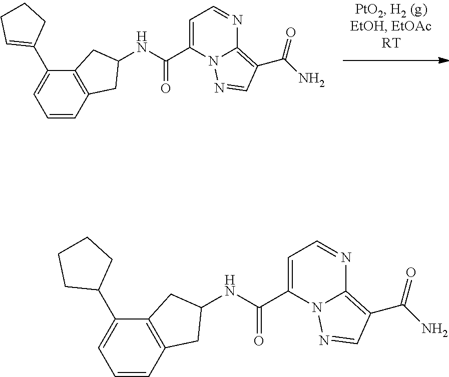 [SMILES: NC(=O)c1cnn2c(C(=O)NC3Cc4cccc(C5=CCCC5)c4C3)ccnc12 ; NC(=O)c1cnn2c(C(=O)NC3Cc4cccc(C5CCCC5)c4C3)ccnc12]

PtO₂ (2.5 mg, 0.01 mmol) was suspended in EtOH (6 mL) and the reaction vessel was evacuated and backfilled with argon (×3). A solution of N7-[4-(cyclopenten-1-yl) indan-2-yl]pyrazolo[1,5-a]pyrimidine-3,7-dicarboxamide (62 mg, 0.16 mmol) in EtOAc (6 mL) was added and the reaction vessel was evacuated and backfilled with argon (×3) then evacuated and backfilled with hydrogen (×3). The reaction was stirred at room temperature under a hydrogen atmosphere (balloon) for 20 h. The reaction mixture was filtered through a short pad of Dicalite® and concentrated in vacuo. The crude product was purified by column chromatography (eluting with 0 to 4% MeOH in DCM) and preparative HPLC (basic and acidic methods) to provide the title compound (5 mg).

¹H NMR (400 MHz, DMSO-d₆) δ 9.91 (d, J=7.0 Hz, 1H), 8.92 (d, J=4.3 Hz, 1H), 8.63 (s, 1H), 7.63 (d, J=4.3 Hz, 1H), 7.58 (br. s, 1H), 7.52 (br. s, 1H), 7.18-7.05 (m, 3H), 4.76 (dt, J=12.8, 6.4 Hz, 1H), 3.45-3.35 (m, 2H), 3.10-2.93 (m, 3H), 2.11-1.89 (m, 2H), 1.84-1.72 (m, 2H), 1.72-1.61 (m, 2H), 1.61-1.45 (m, 2H)

LCMS (Method 2): 2.64 min, 390.2 [M+H]⁺

### Synthesis of compound 16, N7-[4-(4-chlorophenoxy) indan-2-yl]pyrazolo[1,5-a]pyrimidine-3,7-dicarboxamide

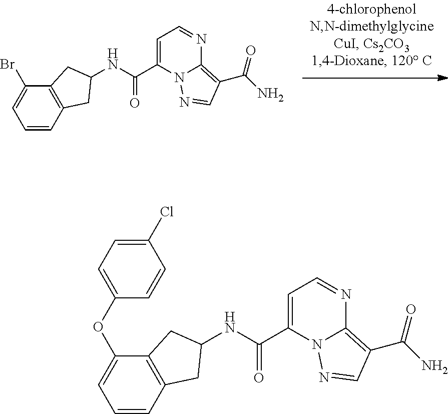 [SMILES: NC(=O)c1cnn2c(C(=O)NC3Cc4cccc(Br)c4C3)ccnc12 ; NC(=O)c1cnn2c(C(=O)NC3Cc4cccc(Oc5ccc(Cl)cc5)c4C3)ccnc12]

N7-(4-bromoindan-2-yl)pyrazolo[1,5-a]pyrimidine-3,7-dicarboxamide (40 mg, 0.10 mmol), 4-chlorophenol (64 mg, 0.50 mmol), Cs₂CO₃ (65 mg, 0.20 mmol), CuI (9 mg, 0.05 mmol) and N,N-dimethylglycine (15 mg, 0.15 mmol) were placed in a vial and the vial was sealed and purged with argon for 5 min. 1,4-Dioxane (2 mL) was added and the reaction was heated at 120° C. for 17 h then cooled to room temperature. The reaction mixture was diluted with EtOAc, filtered through a short pad of Dicalite®, and concentrated in vacuo. The crude product was purified by column chromatography (eluting with 0 to 5% MeOH in DCM) and preparative HPLC (basic method) to provide the title compound (16 mg).

¹H NMR (400 MHz, DMSO-d₆) δ 9.91 (d, J=5.9 Hz, 1H), 8.92 (d, J=4.4 Hz, 1H), 8.65 (s, 1H), 7.62 (d, J=4.3 Hz, 1H), 7.59 (br. s, 1H), 7.52 (br. s, 1H), 7.46-7.35 (m, 2H), 7.26 (t, J=7.7 Hz, 1H), 7.16 (dd, J=7.4, 1.0 Hz, 1H), 7.01-6.93 (m, 2H), 6.84 (dd, J=8.0, 1.0 Hz, 1H), 4.85-4.73 (m, 1H), 3.43 (dd, J=16.4, 7.6 Hz, 1H), 3.20 (dd, J=16.4, 7.6 Hz, 1H), 3.05 (dd, J=16.4, 5.4 Hz, 1H), 2.80 (dd, J=16.4, 5.2 Hz, 1H)

LCMS (Method 2): 2.66 min, 448.1, 450.1 [M+H]⁺

### Synthesis of compound 17, N7-[4-(2-pyridyl) indan-2-yl]pyrazolo[1,5-a]pyrimidine-3,7-dicarboxamide

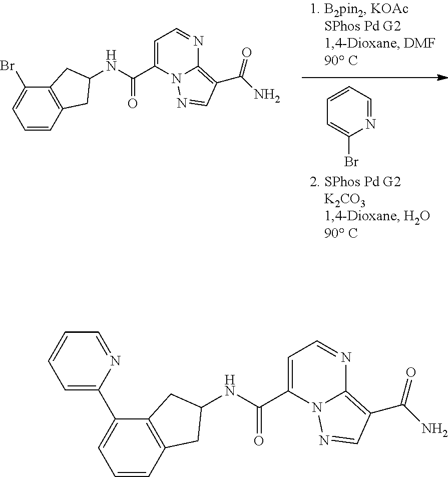 [SMILES: NC(=O)c1cnn2c(C(=O)NC3Cc4cccc(Br)c4C3)ccnc12 ; Brc1ccccn1 ; NC(=O)c1cnn2c(C(=O)NC3Cc4cccc(-c5ccccn5)c4C3)ccnc12]

A mixture of N7-(4-bromoindan-2-yl)pyrazolo[1,5-a]pyrimidine-3,7-dicarboxamide (50 mg, 0.12 mmol), B₂pin₂ (bis(pinacolato)diboron; 63 mg, 0.25 mmol), KOAc (37 mg, 0.37 mmol), and SPhos Pd G2 (9 mg, 0.01 mmol) was purged with argon for 5 min. 1,4-Dioxane (2.5 mL) was added and the mixture was degassed by sparging with a stream of argon for 5 min. The reaction was heated at 90° C. for 20 h then cooled to room temperature. DMF (1 mL) and additional portions of B₂pin₂ (63 mg, 0.25 mmol), KOAc (7 mg, 0.37 mmol), and SPhos Pd G2 (9 mg, 0.01 mmol) were added and the mixture was degassed by sparging with a stream of argon for 5 min. The reaction was heated at 90° C. for a further 20 h then cooled to room temperature. The reaction mixture was diluted with EtOAc and filtered through a short pad of Dicalite®. The filtrate was concentrated in vacuo to give the crude intermediate boronate ester, N7-[4-(4,4,5,5-tetramethyl-1,3,2-dioxaborolan-2-yl) indan-2-yl]pyrazolo[1,5-a]pyrimidine-3,7-dicarboxamide.

A mixture of the crude N7-[4-(4,4,5,5-tetramethyl-1,3,2-dioxaborolan-2-yl) indan-2-yl]pyrazolo[1,5-a]pyrimidine-3,7-dicarboxamide obtained as above, 2-bromopyridine (40 mg, 0.25 mmol), SPhos Pd G2 (12 mg, 0.02 mmol), and K₂CO₃ (70 mg, 0.50 mmol) was purged with argon for 5 min. 1,4-Dioxane (3 mL) and water (300 μL) were added and the mixture was degassed by sparging with a stream of argon for 5 min. The reaction was heated at 90° C. for 20 h then cooled to room temperature. The reaction mixture was diluted with EtOAc and filtered through a short pad of Dicalite®. The filtrate was concentrated in vacuo and the crude product was purified by column chromatography (eluting with 0 to 6% MeOH in DCM) and preparative HPLC (basic method) to provide the title compound (9.2 mg).

¹H NMR (400 MHz, DMSO-d₆) δ 9.91 (d, J=6.7 Hz, 1H), 8.90 (d, J=4.3 Hz, 1H), 8.68 (ddd, J=4.8, 1.9, 0.9 Hz, 1H), 8.63 (s, 1H), 7.95-7.84 (m, 1H), 7.72 (dt, J=8.0, 1.1 Hz, 1H), 7.62 (d, J=4.4 Hz, 1H), 7.60-7.55 (m, 2H), 7.51 (br. s, 1H), 7.42-7.30 (m, 3H), 4.85-4.71 (m, 1H), 3.64 (dd, J=16.8, 7.5 Hz, 1H), 3.45 (dd, J=16.2, 7.6 Hz, 1H), 3.30-3.19 (m, 1H), 3.07 (dd, J=16.2, 5.5 Hz, 1H)

LCMS (Method 2): 1.71 min, 399.2 [M+H]⁺

### Synthesis of tert-butyl N-[cis-3-(trifluoromethoxy)cyclobutyl]carbamate

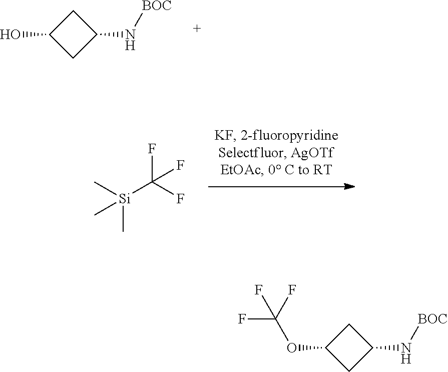

Selectfluor (1-(chloromethyl)-4-fluoro-1,4-diazabicyclo[2.2.2]octane-1,4-diium ditetrafluoroborate; 2.17 g, 6.09 mmol), AgOTf (silver trifluoromethanesulfonate; 4.54 g, 17.67 mmol) and KF (1.03 g, 17.67 mmol) were added to a solution of tert-butyl N-(cis-3-hydroxycyclobutyl) carbamate (827 mg, 4.42 mmol) in EtOAc (30 mL), followed by the dropwise addition of 2-fluoropyridine (1.52 mL, 17.67 mmol) and trimethyl(trifluoromethyl) silane (1.63 mL, 11.04 mmol). The reaction was stirred at room temperature for 60 h, then the mixture was filtered through a short pad of Dicalite® and concentrated in vacuo. The crude product was purified by column chromatography (eluting with 0 to 100% EtOAc in heptane) to provide the title compound (567 mg).

¹H NMR (400 MHZ, CDCl₃) δ 4.65 (s, 1H), 4.36 (p, J=7.2 Hz, 1H), 3.81 (s, 1H), 2.83 (d, J=9.5 Hz, 2H), 2.14-2.02 (m, 2H), 1.44 (s, 9H)

### Synthesis of 2,2,2-trifluoroacetic acid;cis-3-(trifluoromethoxy)cyclobutanamine

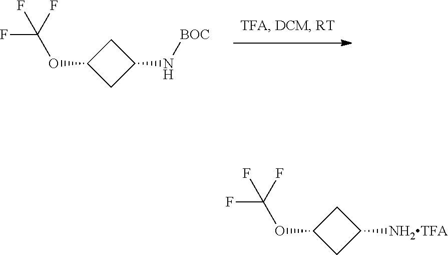

TFA (940 μL, 12.34 mmol) was added dropwise to a solution of tert-butyl N-[cis-3-(trifluoromethoxy)cyclobutyl]carbamate (315 mg, 1.23 mmol) in DCM (2 mL) and the reaction was stirred at room temperature for 2 h. The reaction mixture was concentrated in vacuo and the residue was dissolved in Et₂O (1 mL). Heptane (~4 mL) was added and the mixture was cooled to 0° C. and left to stand for 1 h. The resulting precipitate was collected by filtration, washed with heptane and dried to provide the title compound (260 mg).

¹H NMR (400 MHz, DMSO-d₆) δ 8.21 (s, 3H), 4.71-4.59 (m, 1H), 3.42-3.37 (m, 1H), 2.77-2.65 (m, 2H), 2.41-2.27 (m, 2H)

### Synthesis of compound 18, N7-[cis-3-(trifluoromethoxy)cyclobutyl]pyrazolo[1,5-a]pyrimidine-3,7-dicarboxamide

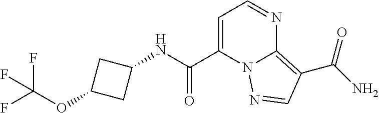 [SMILES: NC(=O)c1cnn2c(C(=O)N[C@H]3C[C@@H](OC(F)(F)F)C3)ccnc12]

N7-[cis-3-(trifluoromethoxy)cyclobutyl]pyrazolo[1,5-a]pyrimidine-3,7-dicarboxamide (36 mg) was synthesised following a similar procedure to that described in General Method B.

¹H NMR (400 MHz, DMSO-d₆) δ 9.92 (d, J=6.5 Hz, 1H), 8.94 (d, J=4.3 Hz, 1H), 8.71 (s, 1H), 7.63 (d, J=4.3 Hz, 1H), 7.61 (s, 1H), 7.54 (s, 1H), 4.77-4.65 (m, 1H), 4.26-4.16 (m, 1H), 2.93-2.82 (m, 2H), 2.43-2.32 (m, 2H)

LCMS (Method 2): 2.04 min, 344.2 [M+H]⁺

### Synthesis of 4-[tert-butyl(dimethyl)silyl]oxypyrrolidin-2-one

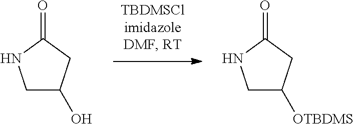 [SMILES: O=C1CC(O)CN1 ; CC(C)(C)[Si](C)(C)OC1CNC(=O)C1]

Imidazole (0.94 g, 13.85 mmol) and tert-butyldimethylsilyl chloride (1.57 g, 10.39 mmol) were added to a solution of 4-hydroxypyrrolidin-2-one (1 g, 9.89 mmol) in DMF (6 mL) and the reaction was stirred at room temperature for 16 h. The reaction mixture was poured into ice water (~200 mL) and 0.2 M aq. HCl (~5 mL) was added. The mixture was stirred for 10 mins then diluted with EtOAc. The layers were separated, and the aqueous layer was extracted with EtOAc (×2). The combined organic layers were washed with brine, dried over Na₂SO₄, and concentrated in vacuo to provide the title compound (2.21 g).

¹H NMR (400 MHZ, CDCl₃) δ 6.02 (s, 1H), 4.55-4.49 (m, 1H), 3.59 (ddd, J=10.0, 6.1, 0.8 Hz, 1H), 3.23 (ddd, J=10.0, 3.4, 1.0 Hz, 1H), 2.54 (dd, J=16.9, 6.8 Hz, 1H), 2.26 (dd, 1H), 0.88 (s, 9H), 0.07 (d, J=1.1 Hz, 6H)

### Synthesis of tert-butyl-[(5-ethoxy-3,4-dihydro-2H-pyrrol-3-yl)oxy]-dimethyl-silane

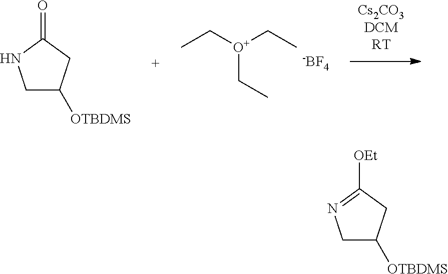

CsCO₃ (9.94 g, 30.51 mmol) and triethyloxonium tetrafluoroborate (2.18 g, 11.49 mmol) were added sequentially to a solution of 4-[tert-butyl(dimethyl)silyl]oxypyrrolidin-2-one (2.19 g, 10.17 mmol) in DCM (66 mL) and the reaction was stirred at room temperature for 23 h. The reaction mixture was diluted with water and stirred for 10 min. The layers were separated, and the organic layer was washed with water (×3), dried over Na₂SO₄, and concentrated in vacuo to provide the title compound (2.24 g).

¹H NMR (400 MHZ, CDCl₃) δ 4.55-4.49 (m, 1H), 4.28-4.12 (m, 2H), 3.85-3.76 (m, 1H), 3.55-3.50 (m, 1H), 2.72-2.63 (m, 1H), 2.45-2.35 (m, 1H), 1.30 (t, J=7.1 Hz, 3H), 0.87 (s, 9H), 0.05 (s, 6H)

### Synthesis of 3-[tert-butyl(dimethyl)silyl]oxy-N-(2,2-diethoxyethyl)-3,4-dihydro-2H-pyrrol-5-amine

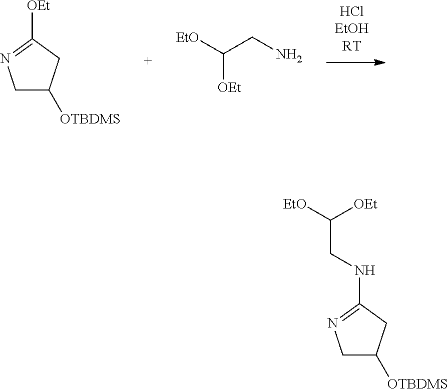 [SMILES: CCOC1=NCC(O[Si](C)(C)C(C)(C)C)C1 ; CCOC(CN)OCC ; CCOC(CNC1=NCC(O[Si](C)(C)C(C)(C)C)C1)OCC]

2,2-Diethoxyethanamine (1.34 mL, 9.20 mmol) was added to a solution of tert-butyl-[(5-ethoxy-3,4-dihydro-2H-pyrrol-3-yl)oxy]-dimethyl-silane (2.24 g, 9.20 mmol) in EtOH (35 mL). HCl (1.25 M in EtOH; 7.36 mL, 9.20 mmol) was added dropwise over 10 min and the reaction was stirred at room temperature for 16 h. The reaction mixture was concentrated in vacuo and the residue was suspended in boiling Et₂O (~50 mL) for 10 min. The mixture was cooled to room temperature and the precipitate was collected by filtration, washed with Et₂O, and dried to provide the title compound (2.68 g).

¹H NMR (400 MHZ, CDCl₃) δ 10.27-10.05 (m, 1H), 4.74-4.53 (m, 2H), 3.89-3.47 (m, 7H), 3.31 (dd, J=7.0, 5.5 Hz, 1H), 3.23-2.74 (m, 2H), 1.24-1.18 (m, 6H), 0.87 (d, J=3.5 Hz, 9H), 0.11-0.04 (m, 6H)

### Synthesis of 6,7-dihydro-5H-pyrrolo[1,2-a]imidazol-6-ol

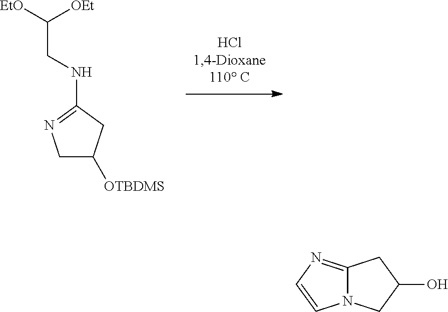 [SMILES: CCOC(CNC1=NCC(O[Si](C)(C)C(C)(C)C)C1)OCC ; OC1Cc2nccn2C1]

0.625 M aq. HCl (8.08 mL, 5.05 mmol) was added to a solution of 3-[tert-butyl(dimethyl)silyl]oxy-N-(2,2-diethoxyethyl)-3,4-dihydro-2H-pyrrol-5-amine (1.67 g, 5.05 mmol) in 1,4-dioxane (1.7 mL) and the reaction was heated at 110° C. for 2 h. The reaction mixture was cooled to room temperature and diluted with Et₂O. The layers were separated, and the aqueous layer was basified with Na₂CO₃ and concentrated in vacuo. The residue was extracted with a 1:9 mixture of MeOH:DCM, and the combined organic layers were dried through a hydrophobic frit and concentrated in vacuo. The residue was dissolved in DCM and the solution was filtered through a short pad of Dicalite® and concentrated in vacuo to provide the title compound (574 mg).

¹H NMR (400 MHz, DMSO-d₆) δ 7.05 (d, J=1.3 Hz, 1H), 6.87 (d, J=1.3 Hz, 1H), 5.51 (s, 1H), 4.85 (tt, J=5.9, 2.9 Hz, 1H), 4.12 (dd, J=11.3, 5.8 Hz, 1H), 3.70 (dd, J=11.3, 2.8 Hz, 1H), 3.01 (dd, J=16.2, 6.5 Hz, 1H), 2.54 (dd, J=16.2, 2.9 Hz, 1H)

### Synthesis of 6-azido-6,7-dihydro-5H-pyrrolo[1,2-a]imidazole

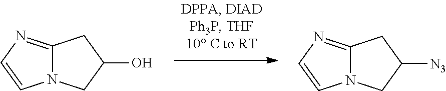 [SMILES: OC1Cc2nccn2C1 ; [N-]=[N+]=NC1Cc2nccn2C1]

Diisopropyl azodicarboxylate (380 μL, 1.93 mmol) was added dropwise over 10 min to a suspension of 6,7-dihydro-5H-pyrrolo[1,2-a]imidazol-6-ol (200 mg, 1.61 mmol) and Ph₃P (507 mg, 1.93 mmol) in THF (7.5 mL) at 10° C. Diphenylphosphoryl azide (418 μL, 1.93 mmol) was added dropwise over 10 min and the reaction was warmed to room temperature and stirred overnight. The reaction mixture was concentrated in vacuo, and the crude product was purified by column chromatography (eluting with 0 to 10% MeOH in DCM) to provide the title compound (226 mg).

¹H NMR (400 MHZ, CDCl₃) δ 7.06 (d, J=1.4 Hz, 1H), 6.88 (d, J=1.4 Hz, 1H), 4.81-4.71 (m, 1H), 4.23 (dd, J=11.4, 6.7 Hz, 1H), 3.91 (dd, J=11.4, 3.8 Hz, 1H), 3.26 (dd, J=16.6, 7.3 Hz, 1H), 2.97 (dd, J=16.6, 4.0 Hz, 1H)

### Synthesis of 6,7-dihydro-5H-pyrrolo[1,2-a]imidazol-6-amine

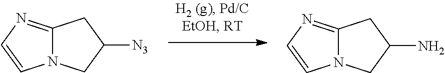 [SMILES: [N-]=[N+]=NC1Cc2nccn2C1 ; NC1Cc2nccn2C1]

10% Palladium on carbon (156 mg, 0.15 mmol) was placed in a flask and the flask was purged with argon. A solution of 6-azido-6,7-dihydro-5H-pyrrolo[1,2-a]imidazole (219 mg, 1.47 mmol) in EtOH (4 mL) was added and the flask was evacuated and backfilled with argon (×3), then evacuated and backfilled with hydrogen (×3). The reaction was stirred under a hydrogen atmosphere (balloon) at room temperature for 1 h. The reaction mixture was filtered through a short pad of Dicalite®, washed with EtOH, and concentrated in vacuo to provide the title compound (178 mg).

¹H NMR (400 MHZ, CDCl₃) δ 7.04 (d, J=1.3 Hz, 1H), 6.85 (d, J=1.3 Hz, 1H), 4.30-4.17 (m, 2H), 3.65 (dd, J=10.5, 4.7 Hz, 1H), 3.21 (dd, J=16.1, 7.3 Hz, 1H), 2.61 (dd, J=16.1, 5.1 Hz, 1H). Exchangeable protons not observed.

### Synthesis of compound 19, N7-(6,7-dihydro-5H-pyrrolo[1,2-a]imidazol-6-yl)pyrazolo[1,5-a]pyrimidine-3,7-dicarboxamide

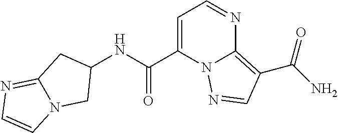 [SMILES: NC(=O)c1cnn2c(C(=O)NC3Cc4nccn4C3)ccnc12]

N7-(6,7-dihydro-5H-pyrrolo[1,2-a]imidazol-6-yl)pyrazolo[1,5-a]pyrimidine-3,7-dicarboxamide (18 mg) was synthesised following a similar procedure to that described in General Method B.

¹H NMR (400 MHz, DMSO-d₆) δ 10.14 (d, J=7.1 Hz, 1H), 8.94 (d, J=4.4 Hz, 1H), 8.67 (s, 1H), 7.65 (d, J=4.3 Hz, 1H), 7.60 (s, 1H), 7.52 (s, 1H), 7.12 (d, J=1.3 Hz, 1H), 6.92 (d, J=1.3 Hz, 1H), 5.29-5.16 (m, 1H), 4.41 (dd, J=11.2, 7.5 Hz, 1H), 3.99 (dd, J=11.2, 4.4 Hz, 1H), 3.27 (dd, 1H), 2.86 (dd, J=16.3, 4.6 Hz, 1H)

LCMS (Method 2): 1.00 min, 312.2 [M+H]⁺

### Synthesis of methyl 3-(2-amino-3-pyridyl)-2-(tert-butoxycarbonylamino) prop-2-enoate

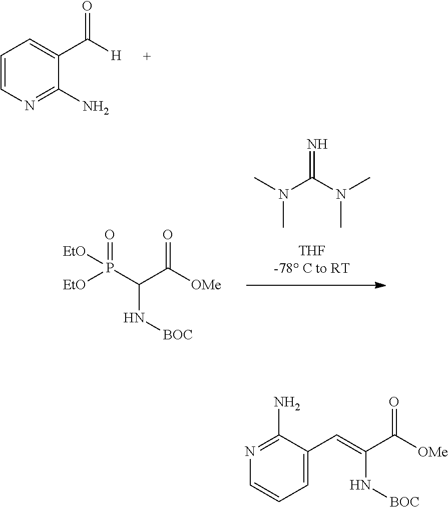

1,1,3,3-Tetramethylguanidine (2.32 mL, 18.50 mmol) was added to a solution of methyl 2-(tert-butoxycarbonylamino)-2-dimethoxyphosphoryl-acetate (5 g, 16.82 mmol) in THF (50 mL) at −78° C. and the reaction was stirred for 15 min. A solution of 2-aminopyridine-3-carbaldehyde (2.05 g, 16.82 mmol) in THF (20 mL) was added, and the reaction was warmed to room temperature and stirred for 18 h. The reaction mixture was diluted with EtOAc and washed with water and brine. The organic layer was dried over Na₂SO₄ and concentrated in vacuo. The crude product was purified by column chromatography (eluting with 0 to 100% EtOAc in heptane) to provide the title compound (2.43 g).

¹H NMR (400 MHz, DMSO-d₆) δ 8.68-8.41 (m, 1H), 7.93 (dd, J=4.8, 1.8 Hz, 1H), 7.66-7.62 (m, 1H), 7.02 (s, 1H), 6.58 (dd, J=7.6, 4.8 Hz, 1H), 6.09 (s, 2H), 3.73 (s, 3H), 1.40 (s, 9H)

LCMS (Method 1): 0.68 min, 294.2 [M+H]⁺

### Synthesis of tert-butyl N-(2-oxo-3,4-dihydro-1H-1,8-naphthyridin-3-yl) carbamate

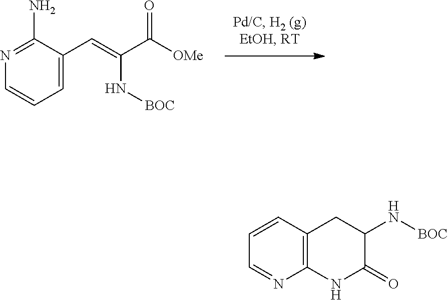 [SMILES: COC(=O)/C(=C/c1cccnc1N)NC(=O)OC(C)(C)C ; CC(C)(C)OC(=O)NC1Cc2cccnc2NC1=O]

10% Pd/C (palladium on carbon; 689 mg, 0.65 mmol) was added to a solution of methyl 3-(2-amino-3-pyridyl)-2-(tert-butoxycarbonylamino) prop-2-enoate (2 g, 6.48 mmol) in EtOH (30 mL) under an atmosphere of argon. A balloon of hydrogen was attached to the flask and the reaction was stirred at room temperature for 65 h. The reaction mixture was filtered through a short pad of Dicalite®, washed with EtOAc, and concentrated in vacuo. The crude product was purified twice by column chromatography (eluting first with 0 to 100% EtOAc in heptane and then with 10% MeOH in DCM) to provide the title compound (700 mg).

¹H NMR (400 MHz, DMSO-d₆) δ 10.59 (s, 1H), 8.14-8.08 (m, 1H), 7.66-7.54 (m, 1H), 7.03 (d, J=8.5 Hz, 1H), 6.96 (dd, J=7.4, 5.0 Hz, 1H), 4.22 (dt, J=14.7, 7.7 Hz, 1H), 3.06-2.86 (m, 2H), 1.41 (s, 9H)

LCMS (Method 1): 0.98 min, 264.2 [M+H]⁺

### Synthesis of 3-amino-3,4-dihydro-1H-1,8-naphthyridin-2-one hydrochloride

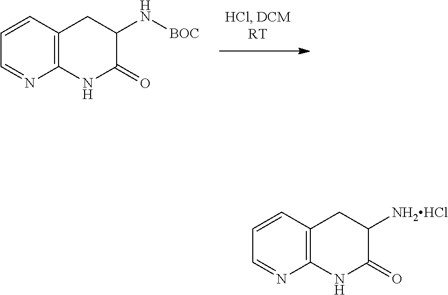

HCl (4 M in 1,4-dioxane; 3.32 mL, 13.29 mmol) was added to a solution of tert-butyl N-(2-oxo-3,4-dihydro-1H-1,8-naphthyridin-3-yl) carbamate (700 mg, 2.66 mmol) in DCM (5 mL) and the reaction was stirred at room temperature for 1 h then concentrated in vacuo. The residue was diluted with Et₂O and the resulting precipitate was collected by filtration, washed with Et₂O, and dried to provide the title compound (530 mg).

¹H NMR (400 MHz, DMSO-d₆) δ 11.14 (s, 1H), 8.66 (d, J=5.7 Hz, 3H), 8.22-8.15 (m, 1H), 7.73 (dt, J=7.6, 1.4 Hz, 1H), 7.05 (dd, J=7.4, 5.0 Hz, 1H), 4.32 (dt, J=14.4, 6.2 Hz, 1H), 3.25 (dd, J=15.4, 6.8 Hz, 1H), 3.16-3.01 (m, 1H)

### Synthesis of 1,2,3,4-tetrahydro-1,8-naphthyridin-3-amine dihydrochloride

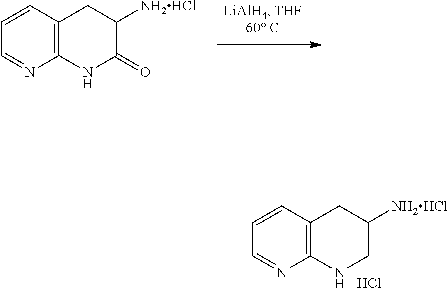

LiAlH₄ (2.4 M in THF; 7 mL, 16.80 mmol) was added dropwise to a suspension of 3-amino-3,4-dihydro-1H-1,8-naphthyridin-2-one hydrochloride (530 mg, 2.65 mmol) in THF (10 mL) and the reaction was heated at 60° C. for 3 h. The reaction mixture was cooled to room temperature and then to 0° C. and quenched with water (500 μL), 2 M aq. NaOH (500 μL) and water (1.5 mL). The mixture was warmed to room temperature, MgSO₄ was added, and the suspension was stirred rapidly for 30 min. The solids were removed by filtration and the filtrate was concentrated in vacuo. The crude product was purified by SCX chromatography (eluting with 2 M NH₃ in MeOH). The basic fractions were concentrated in vacuo, and the residue was dissolved in DCM and treated with HCl (4 M in 1,4-dioxane). The resulting precipitate was collected by filtration, washed with Et₂O, and dried to provide the title compound (99 mg).

¹H NMR (400 MHz, DMSO-d₆) δ 8.25 (s, 3H), 7.85 (dd, J=5.0, 1.8 Hz, 1H), 7.32 (dd, J=7.2, 1.7 Hz, 1H), 6.72 (s, 1H), 6.55 (dd, J=7.2, 5.0 Hz, 1H), 3.60 (s, 1H), 3.52-3.44 (m, 1H), 3.30-3.26 (m, 1H), 3.05 (dd, J=16.6, 4.9 Hz, 1H), 2.80 (dd, J=16.6, 6.6 Hz, 1H).

Additional exchangeable proton not observed.

### Synthesis of compound 20, N7-(1,2,3,4-tetrahydro-1,8-naphthyridin-3-yl)pyrazolo[1,5-a]pyrimidine-3,7-dicarboxamide

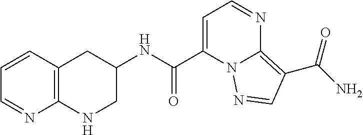 [SMILES: NC(=O)c1cnn2c(C(=O)NC3CNc4ncccc4C3)ccnc12]

N7-(1,2,3,4-tetrahydro-1,8-naphthyridin-3-yl)pyrazolo[1,5-a]pyrimidine-3,7-dicarboxamide (5 mg) was synthesised following a similar procedure to that described in General Method B.

¹H NMR (400 MHz, DMSO-d₆) δ 9.96 (d, J=7.1 Hz, 1H), 8.94 (d, J=4.3 Hz, 1H), 8.55 (s, 1H), 7.83 (dd, J=5.0, 1.8 Hz, 1H), 7.72 (d, J=4.3 Hz, 1H), 7.60 (s, 1H), 7.53 (s, 1H), 7.28-7.23 (m, 1H), 6.57 (d, J=2.7 Hz, 1H), 6.50 (dd, J=7.2, 4.9 Hz, 1H), 4.44 (s, 1H), 3.54 (d, J=11.9 Hz, 1H), 3.38-3.34 (m, 1H), 3.09 (dd, J=16.1, 4.6 Hz, 1H), 2.88 (dd, J=16.2, 6.2 Hz, 1H)

LCMS (Method 2): 1.20 min, 338.2 [M+H]⁺

### Synthesis of compound 96, N7-[2-(3-methylphenoxy)butyl]pyrazolo[1,5-a]pyrimidine-3,7-dicarboxamide

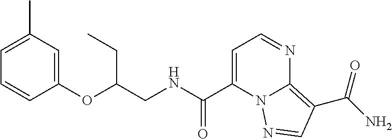 [SMILES: CCC(CNC(=O)c1ccnc2c(C(N)=O)cnn12)Oc1cccc(C)c1]

N7-[2-(3-methylphenoxy)butyl]pyrazolo[1,5-a]pyrimidine-3,7-dicarboxamide was synthesised following a similar procedure to that described in General Method B.

¹H NMR (400 MHz, DMSO-d₆) δ 10.08 (t, J=5.8 Hz, 1H), 8.94 (d, J=4.3 Hz, 1H), 8.67 (s, 1H), 7.70 (d, J=4.3 Hz, 1H), 7.61 (s, 1H), 7.54 (s, 1H), 7.14 (t, J=7.7 Hz, 1H), 6.89-6.80 (m, 2H), 6.75-6.68 (m, 1H), 4.56-4.46 (m, 1H), 3.78-3.62 (m, 2H), 2.24 (s, 3H), 1.77-1.63 (m, 2H), 0.97 (t, J=7.4 Hz, 3H)

LCMS (Method 7): 2.64 min, 368.2 [M+H]⁺

### Synthesis of compound 97, N7-[2-methyl-5-(pyridin-4-yl)phenyl]pyrazolo[1,5-a]pyrimidine-3,7-dicarboxamide

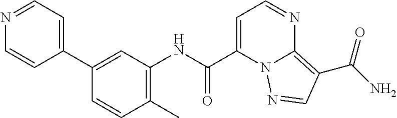 [SMILES: Cc1ccc(-c2ccncc2)cc1NC(=O)c1ccnc2c(C(N)=O)cnn12]

N7-[2-methyl-5-(pyridin-4-yl)phenyl]pyrazolo[1,5-a]pyrimidine-3,7-dicarboxamide was synthesised following a similar procedure to that described in General Method B.

LCMS (Method 7): 1.64 min, 373.2 [M+H]⁺

### Synthesis of compound 98, N7-[1-(5-methoxy-1H-1,3-benzodiazol-2-yl)ethyl]pyrazolo[1,5-a]pyrimidine-3,7-dicarboxamide

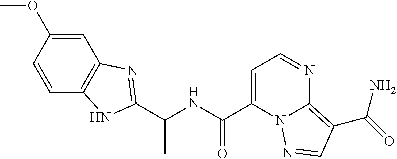 [SMILES: COc1ccc2[nH]c(C(C)NC(=O)c3ccnc4c(C(N)=O)cnn34)nc2c1]

N7-[1-(5-methoxy-1H-1,3-benzodiazol-2-yl)ethyl]pyrazolo[1,5-a]pyrimidine-3,7-dicarboxamide was synthesised following a similar procedure to that described in General Method B.

LCMS (Method 7): 1.45 min, 380.2 [M+H]⁺

### Synthesis of compound 99, N7-[1-(4-tert-butylphenoxy) propan-2-yl]pyrazolo[1,5-a]pyrimidine-3,7-dicarboxamide

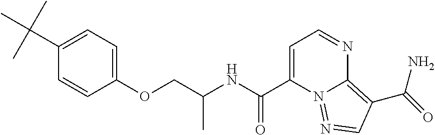

N7-[1-(4-tert-butylphenoxy) propan-2-yl]pyrazolo[1,5-a]pyrimidine-3,7-dicarboxamide synthesised following a similar procedure to that described in General Method B.

LCMS (Method 7): 2.95 min, 396.2 [M+H]⁺

### Synthesis of compound 100, N7-(3,3,3-trifluoropropyl)pyrazolo[1,5-a]pyrimidine-3,7-dicarboxamide

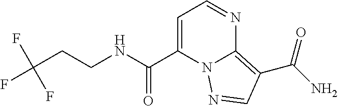 [SMILES: NC(=O)c1cnn2c(C(=O)NCCC(F)(F)F)ccnc12]

N7-(3,3,3-trifluoropropyl)pyrazolo[1,5-a]pyrimidine-3,7-dicarboxamide was synthesised following a similar procedure to that described in General Method B.

LCMS (Method 7): 1.91 min, 302.2 [M+H]⁺

### Synthesis of compound 101, N7-[(4,4-difluorocyclohexyl)methyl]pyrazolo[1,5-a]pyrimidine-3,7-dicarboxamide

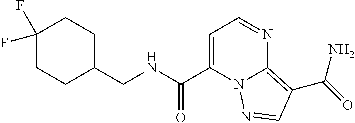 [SMILES: NC(=O)c1cnn2c(C(=O)NCC3CCC(F)(F)CC3)ccnc12]

N7-[(4,4-difluorocyclohexyl)methyl]pyrazolo[1,5-a]pyrimidine-3,7-dicarboxamide synthesised following a similar procedure to that described in General Method B.

LCMS (Method 7): 2.21 min, 338.2 [M+H]⁺

### Synthesis of compound 102, N7-(3,3-difluorocyclobutyl)pyrazolo[1,5-a]pyrimidine-3,7-dicarboxamide

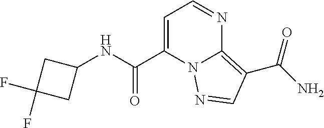 [SMILES: NC(=O)c1cnn2c(C(=O)NC3CC(F)(F)C3)ccnc12]

N7-(3,3-difluorocyclobutyl)pyrazolo[1,5-a]pyrimidine-3,7-dicarboxamide (106 mg) was synthesised following a similar procedure to that described in General Method B.

¹H NMR (400 MHz, MeOD) δ 8.95 (d, J=4.3 Hz, 1H), 8.74 (s, 1H), 7.86 (d, J=4.4 Hz, 1H), 4.51-4.40 (m, 1H), 3.20-3.01 (m, 2H), 2.91-2.76 (m, 2H). Exchangeable protons not observed.

LCMS (Method 2): 1.74 min, 296.2 [M+H]⁺

### Synthesis of compound 103, N7-(4,4-difluorocyclohexyl)pyrazolo[1,5-a]pyrimidine-3,7-dicarboxamide

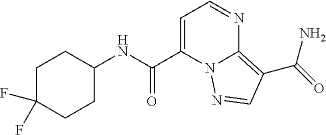 [SMILES: NC(=O)c1cnn2c(C(=O)NC3CCC(F)(F)CC3)ccnc12]

N7-(4,4-difluorocyclohexyl)pyrazolo[1,5-a]pyrimidine-3,7-dicarboxamide was synthesised following a similar procedure to that described in General Method b.

LCMS (Method 7): 2.09 min, 324.2 [M+H]⁺

### Synthesis of ethyl 2-carbamoylpyrazolo[1,5-a]pyrimidine-7-carboxylate

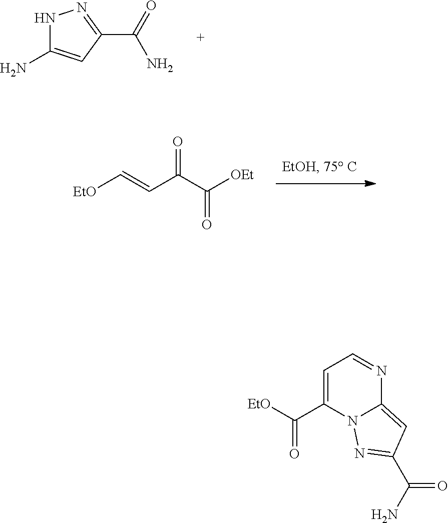 [SMILES: NC(=O)c1cc(N)[nH]n1 ; CCO/C=C/C(=O)C(=O)OCC ; CCOC(=O)c1ccnc2cc(C(N)=O)nn12]

Ethyl (E)-4-ethoxy-2-oxo-but-3-enoate (1.365 g, 7.93 mmol) and 5-amino-1H-pyrazole-3-carboxamide (1 g, 7.93 mmol) were dissolved in EtOH (9.5 mL) and the reaction was heated at 75° C. for 2 h. The reaction mixture was cooled to room temperature and concentrated in vacuo. MeOH was added to the residue, and the resulting precipitate was collected by filtration, washed with MeOH, and dried to provide the title compound (1.14 g).

¹H NMR (400 MHz, DMSO-d₆) δ 8.75 (d, J=4.1 Hz, 1H), 7.75 (s, 1H), 7.70 (s, 1H), 7.56 (d, J=4.1 Hz, 1H), 7.24 (s, 1H), 4.50 (q, J=7.1 Hz, 2H), 1.39 (t, J=7.1 Hz, 3H)

LCMS (Method 2): 1.52 min, 235.2 [M+H]⁺

### Synthesis of ethyl 2-cyanopyrazolo[1,5-a]pyrimidine-7-carboxylate

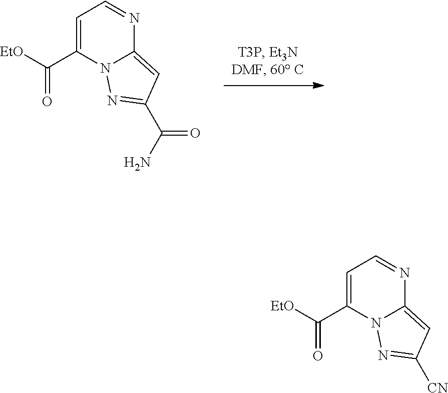 [SMILES: CCOC(=O)c1ccnc2cc(C(N)=O)nn12 ; CCOC(=O)c1ccnc2cc(C#N)nn12]

1-Propanephosphonic anhydride (50 wt % in DMF; 12.2 mL, 20.49 mmol) and Et₃N (2.86 mL, 20.49 mmol) were added to a solution of ethyl 2-carbamoylpyrazolo[1,5-a]pyrimidine-7-carboxylate (600 mg, 2.56 mmol) in DMF (15 mL) and the reaction was heated at 60° C. for 18 h. The reaction mixture was cooled to room temperature and diluted with water. The layers were separated, and the aqueous layer was extracted with DCM (×3). The combined organic layers were concentrated in vacuo and the residue was dissolved in EtOAc. The solution was washed with sat. aq. LiCl (×3), dried through a hydrophobic frit, and concentrated in vacuo to provide the title compound (611 mg). The crude product was taken forward to the next step without purification.

¹H NMR (400 MHz, DMSO-d₆) δ 8.90 (d, J=4.2 Hz, 1H), 7.76 (d, J=4.2 Hz, 1H), 7.74 (s, 1H), 4.50 (q, J=7.1 Hz, 2H), 1.38 (t, J=7.1 Hz, 3H)

LCMS (Method 2): 1.95 min, 217.2 [M+H]⁺

### Synthesis of ethyl 3-bromo-2-cyano-pyrazolo[1,5-a]pyrimidine-7-carboxylate

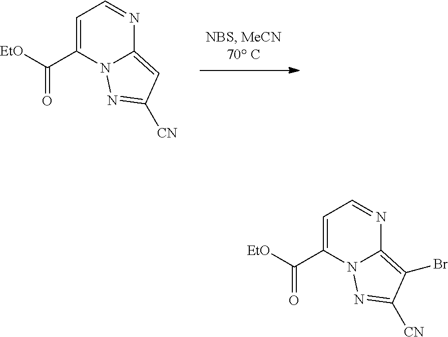 [SMILES: CCOC(=O)c1ccnc2cc(C#N)nn12 ; CCOC(=O)c1ccnc2c(Br)c(C#N)nn12]

NBS (553 mg, 3.11 mmol) was added to a solution of ethyl 2-cyanopyrazolo[1,5-a]pyrimidine-7-carboxylate (611 mg, 2.83 mmol) in MeCN (40 mL) and the reaction was heated at 70° C. for 24 h. The reaction mixture was cooled to room temperature, diluted with DCM, and washed with sat. aq. NaHCO₃ (×3). The organic layer was dried through a hydrophobic frit and concentrated in vacuo to provide the title compound (704 mg).

¹H NMR (400 MHz, DMSO-d₆) δ 8.96 (d, J=4.2 Hz, 1H), 7.85 (d, J=4.2 Hz, 1H), 4.49 (q, J=7.1 Hz, 2H), 1.38 (t, J=7.1 Hz, 3H)

LCMS (Method 2): 2.25 min, 295.0, 297.0 [M+H]⁺

### Synthesis of 3-bromo-2-cyano-pyrazolo[1,5-a]pyrimidine-7-carboxylic acid

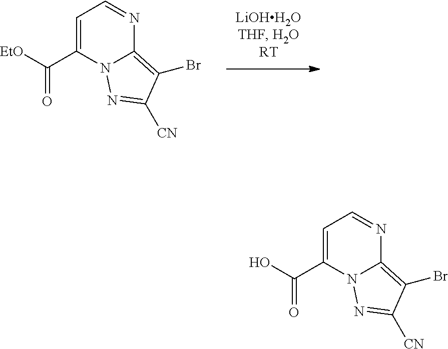 [SMILES: CCOC(=O)c1ccnc2c(Br)c(C#N)nn12 ; N#Cc1nn2c(C(=O)O)ccnc2c1Br]

A solution of LiOH·H₂O (110 mg, 2.62 mmol) in water (6 mL) was added to a solution of ethyl 3-bromo-2-cyano-pyrazolo[1,5-a]pyrimidine-7-carboxylate (704 mg, 2.39 mmol) in THF (6 mL) and the reaction was stirred at room temperature for 18 h. The reaction mixture was acidified to pH 2 with 1 M aq. HCl and extracted with EtOAc (×3). The combined organic layers were dried through a hydrophobic frit and concentrated in vacuo to provide the title compound (525 mg).

¹H NMR (400 MHz, DMSO-d₆) δ 8.93 (d, J=4.2 Hz, 1H), 7.75 (d, J=4.2 Hz, 1H). Exchangeable proton not observed.

LCMS (Method 2): 1.40 min (product does not ionise)

### Synthesis of 3-bromo-2-cyano-N-indan-2-yl-pyrazolo[1,5-a]pyrimidine-7-carboxamide

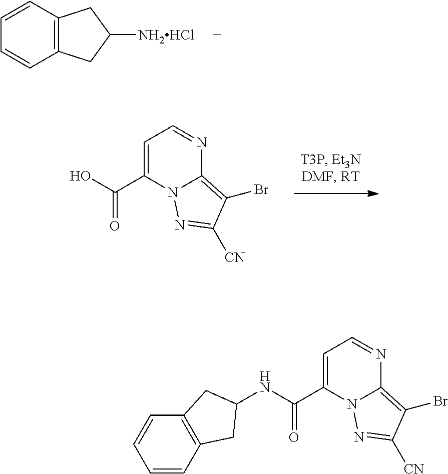

1-Propanephosphonic anhydride (50 wt % in DMF; 2.3 mL, 3.93 mmol) and Et₃N (1.37 mL, 9.83 mmol) were added to a solution of 3-bromo-2-cyano-pyrazolo[1,5-a]pyrimidine-7-carboxylic acid (525 mg, 1.97 mmol) and indan-2-amine hydrochloride (500 mg, 2.95 mmol) in DMF (9.83 mL) and the reaction was stirred at room temperature for 48 h. Additional portions of indan-2-amine hydrochloride (166 mg, 0.98 mmol), 1-propanephosphonic anhydride (50 wt % in DMF; 0.72 mL, 1.23 mmol), and Et₃N (0.46 mL, 3.30 mmol) were added and the reaction mixture was stirred at room temperature for a further 4 h. The reaction mixture was diluted with water, and the resulting precipitate was collected by filtration, washed with water, and dried to provide the title compound (549 mg).

¹H NMR (400 MHz, DMSO-d₆) δ 9.46 (d, J=7.2 Hz, 1H), 8.94 (d, J=4.2 Hz, 1H), 7.68 (d, J=4.2 Hz, 1H), 7.30-7.25 (m, 2H), 7.20-7.15 (m, 2H), 4.77 (d, J=7.3 Hz, 1H), 3.40-3.29 (m, 2H), 2.97 (dd, J=16.1, 5.3 Hz, 2H)

LCMS (Method 2): 2.60 min, 382.0, 384.0 [M+H]⁺

### Synthesis of (2,5-dioxopyrrolidin-1-yl) formate

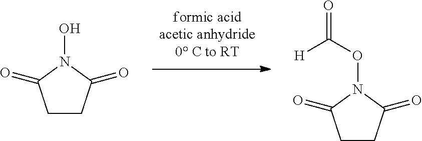 [SMILES: O=C1CCC(=O)N1O ; O=CON1C(=O)CCC1=O]

Acetic anhydride (72 mL, 764.62 mmol) was added slowly over 10 min to formic acid (36 mL, 955.77 mmol) at 0° C. The reaction mixture was warmed to room temperature and stirred for 1.5 h. 1-Hydroxypyrrolidine-2,5-dione (11 g, 95.58 mmol) was added, and the reaction was stirred at room temperature overnight then concentrated in vacuo. The residue was suspended in heptane, collected by filtration, and dried to provide the title compound (13.52 g).

¹H NMR (400 MHz, DMSO-d₆) δ 8.78 (s, 1H), 2.82 (s, 4H)

### Synthesis of compound 21, 2-cyano-N7-indan-2-yl-pyrazolo[1,5-a]pyrimidine-3,7-dicarboxamide

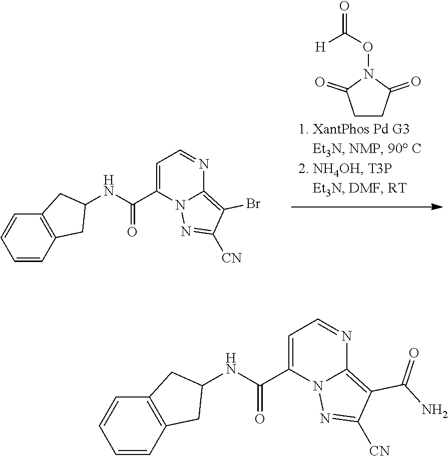 [SMILES: O=CON1C(=O)CCC1=O ; N#Cc1nn2c(C(=O)NC3Cc4ccccc4C3)ccnc2c1Br ; N#Cc1nn2c(C(=O)NC3Cc4ccccc4C3)ccnc2c1C(N)=O]

3-Bromo-2-cyano-N-indan-2-yl-pyrazolo[1,5-a]pyrimidine-7-carboxamide (130 mg, 0.34 mmol) and XantPhos Pd G3 ([(4,5-bis(diphenylphosphino)-9,9-dimethylxanthene)-2-(2′-amino-1,1′-biphenyl)]palladium (II) methanesulfonate; 32.26 mg, 0.03 mmol) were placed in a vial, which was then sealed and purged with argon for 5 min. NMP (3.4 mL) and Et₃N (140 μL, 1.02 mmol) were added and the mixture was degassed by sparging with a stream of argon for 5 min. (2,5-Dioxopyrrolidin-1-yl) formate (243 mg, 1.70 mmol) was added and the vial was sealed immediately. The reaction was heated at 90° C. in a microwave reactor for 1 h then cooled to room temperature. Et₃N (140 μL, 1.02 mmol), 1-propanephosphonic anhydride (50 wt % in DMF; 400 μL, 0.68 mmol), and NH₄OH (240 μL, 3.40 mmol) were added and the reaction was stirred at room temperature for 18 h. The reaction mixture was diluted with EtOAc and water and the layers were separated. The aqueous layer was extracted with EtOAc, and the combined organic layers were washed with sat. aq. LiCl, dried through a hydrophobic frit, and concentrated in vacuo. The crude product was purified by preparative HPLC (acidic and basic methods) to provide the title compound (34 mg).

¹H NMR (400 MHz, DMSO-d₆) δ 9.49 (d, J=7.2 Hz, 1H), 9.06 (d, J=4.3 Hz, 1H), 8.00 (s, 1H), 7.75 (d, J=4.3 Hz, 1H), 7.57 (s, 1H), 7.31-7.25 (m, 2H), 7.21-7.15 (m, 2H), 4.84-4.74 (m, 1H), 3.36 (dd, J=16.1, 7.5 Hz, 2H), 2.98 (dd, J=16.1, 5.3 Hz, 2H)

LCMS (Method 4) 2.08 min, 347.2 [M+H]⁺

### Synthesis of ethyl 2-bromopyrazolo[1,5-a]pyrimidine-7-carboxylate

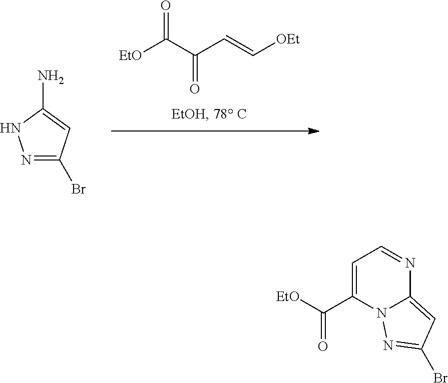 [SMILES: Nc1cc(Br)n[nH]1 ; CCO/C=C/C(=O)C(=O)OCC ; CCOC(=O)c1ccnc2cc(Br)nn12]

3-Bromo-1H-pyrazol-5-amine (10 g, 61.73 mmol) and ethyl (E)-4-ethoxy-2-oxo-but-3-enoate (15.94 g, 92.60 mmol) were dissolved in EtOH (250 mL) and the reaction was stirred at room temperature for 2 h then heated at 78° C. overnight. The reaction mixture was cooled to room temperature and concentrated in vacuo. The crude product was purified by column chromatography (eluting with 0 to 20% EtOAc in heptane) to provide the title compound (11.82 g).

¹H NMR (400 MHZ, CDCl₃) δ 8.55 (d, J=4.2 Hz, 1H), 7.27 (d, J=4.2 Hz, 1H), 6.86 (s, 1H), 4.54 (q, J=7.1 Hz, 2H), 1.44 (t, J=7.1 Hz, 3H)

LCMS (Method 1): 1.61 min, 270.2, 272.2 [M+H]⁺

### Synthesis of 2-bromopyrazolo[1,5-a]pyrimidine-7-carboxylic Acid

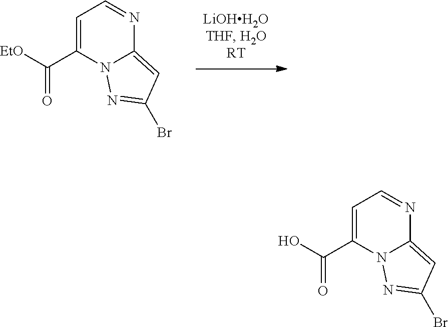 [SMILES: CCOC(=O)c1ccnc2cc(Br)nn12 ; O=C(O)c1ccnc2cc(Br)nn12]

Ethyl 2-bromopyrazolo[1,5-a]pyrimidine-7-carboxylate (4 g, 14.81 mmol) and LiOH. H₂O (622 mg, 14.81 mmol) were dissolved in a mixture of THF (10 mL), and water (2.5 mL) and the reaction was stirred at room temperature overnight. The reaction mixture was diluted with EtOAc and water and the layers were separated. The aqueous layer was acidified to pH 3 with 1 M aq. HCl and extracted with EtOAc (×3). The combined organic layers were dried over Na₂SO₄ and concentrated in vacuo to provide the title compound (3.5 g).

¹H NMR (400 MHz, DMSO-d₆) δ 8.70 (d, J=4.2 Hz, 1H), 7.42 (d, J=4.2 Hz, 1H), 7.10 (s, 1H). Exchangeable proton not observed.

### Synthesis of 2-bromo-N-indan-2-yl-pyrazolo[1,5-a]pyrimidine-7-carboxamide

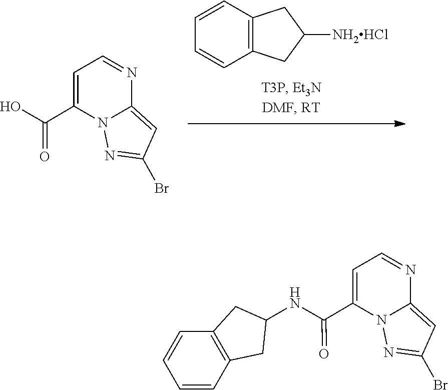

1-Propanephosphonic anhydride (50 wt % in EtOAc; 7.3 mL, 12.40 mmol) was added to a solution of 2-bromopyrazolo[1,5-a]pyrimidine-7-carboxylic acid (2 g, 8.26 mmol), indan-2-amine hydrochloride (1.68 g, 9.92 mmol), and Et₃N (3.46 mL, 24.79 mmol) in DMF (25 mL) and the reaction was stirred at room temperature overnight. The reaction mixture was diluted with water, and the resulting precipitate was collected by filtration and washed with water and heptane to provide the title compound (2.21 g).

¹H NMR (400 MHz, DMSO-d₆) δ 9.72 (d, J=7.1 Hz, 1H), 8.71 (d, J=4.3 Hz, 1H), 7.43 (d, J=4.3 Hz, 1H), 7.28 (dt, J=7.3, 3.7 Hz, 2H), 7.18 (dd, J=5.6, 3.2 Hz, 2H), 7.11 (s, 1H), 4.70-4.82 (m, 1H), 3.37 (dd, J=16.2, 7.6 Hz, 2H), 2.96 (dd, J=16.1, 5.3 Hz, 2H)

### Synthesis of N-indan-2-yl-2-(4,4,5,5-tetramethyl-1,3,2-dioxaborolan-2-yl)pyrazolo[1,5-a]pyrimidine-7-carboxamide

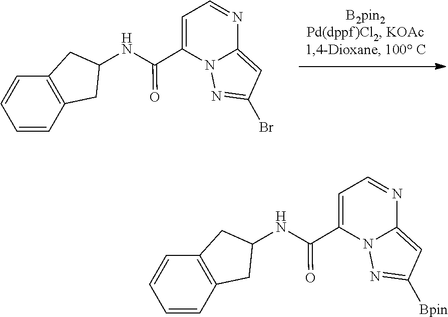

2-Bromo-N-indan-2-yl-pyrazolo[1,5-a]pyrimidine-7-carboxamide (500 mg, 1.40 mmol), B₂pin₂ (427 mg, 1.68 mmol) and KOAc (412 mg, 4.20 mmol) were dissolved in 1,4-dioxane (20 mL) and the solution was degassed by sparging with a stream of argon. Pd(dppf)Cl₂ (102 mg, 0.14 mmol) was added and the solution was degassed with argon for a further 10 min. The reaction was heated at 100° C. for 16 h, then cooled to room temperature and diluted with EtOAc and water. The reaction mixture was filtered through a short pad of of Dicalite® and washed with EtOAc. The layers were separated, and the organic layer was washed with brine, dried over Na₂CO₃, and concentrated in vacuo to provide the title compound (1 g). The crude product was taken forward to the next step without further purification.

LCMS (Method 1): 1.52 min, 323.2 [M+H]⁺ (boronic acid)

### Synthesis of N-indan-2-yl-2-pyridazin-3-yl-pyrazolo[1,5-a]pyrimidine-7-carboxamide

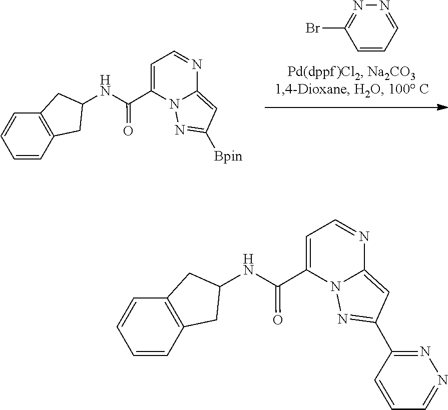

3-Bromopyridazine (288 mg, 1.81 mmol), Na₂CO₃ (393 mg, 3.71 mmol) and water (2.7 mL) were added to a solution of N-indan-2-yl-2-(4,4,5,5-tetramethyl-1,3,2-dioxaborolan-2-yl)pyrazolo[1,5-a]pyrimidine-7-carboxamide (500 mg, 1.24 mmol) in 1,4-dioxane (25 mL). The solution was degassed by sparging with a stream of argon for 10 min. Pd(dppf)Cl₂ (90 mg, 0.12 mmol) was added and the solution was degassed with argon for a further 10 min.

The reaction was heated at 100° C. for 18 h then cooled to room temperature. The reaction mixture was diluted with EtOAc, filtered through a short pad of Dicalite®, and washed with EtOAc. The combined filtrate and washings were washed with water and brine, dried over Na₂SO₄, and concentrated in vacuo. The crude product was purified by column chromatography (eluting with 20 to 100% EtOAc in heptane) to provide the title compound (162 mg).

¹H NMR (400 MHz, DMSO-d₆) δ 10.29 (d, J=7.8 Hz, 1H), 9.29 (dd, J=4.9, 1.7 Hz, 1H), 8.79 (d, J=4.2 Hz, 1H), 7.81 (dd, J=8.5, 4.9 Hz, 1H), 7.67-7.63 (m, 2H), 7.58 (s, 1H), 7.36 (dd, J=5.4, 3.3 Hz, 2H), 7.30-7.22 (m, 2H), 4.94-4.82 (m, 1H), 3.37 (dd, J=16.1, 6.7 Hz, 2H), 3.05 (dd, J=16.0, 3.7 Hz, 2H)

LCMS (Method 5): 0.97 min, 357.2 [M+H]⁺

### Synthesis of 3-bromo-N-indan-2-yl-2-pyridazin-3-yl-pyrazolo[1,5-a]pyrimidine-7-carboxamide

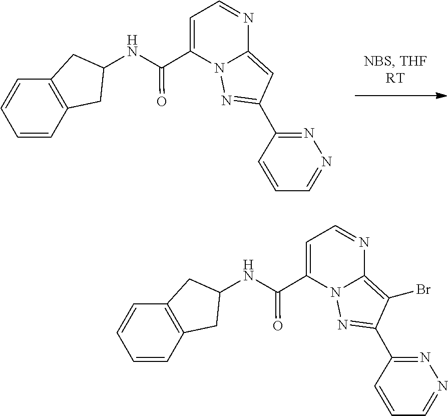

NBS (25 mg, 0.14 mmol) was added to a solution of N-indan-2-yl-2-pyridazin-3-yl-pyrazolo[1,5-a]pyrimidine-7-carboxamide (50 mg, 0.14 mmol) in THF (1.5 mL) and the reaction was stirred at room temperature for 4 h. An additional portion of NBS (10 mg, 0.06 mmol) was added and the reaction was stirred at room temperature for a further 1 h. The reaction mixture was diluted with EtOAc and washed with sat. aq. NaHCO₃. The aqueous layer was extracted with EtOAc (×2), and the combined organic layers were washed with sat. aq. NaHCO₃, dried over Na₂SO₄, and concentrated in vacuo. The crude product was purified by column chromatography (eluting with 20 to 100% EtOAc in heptane) to provide the title compound (44 mg).

¹H NMR (400 MHz, DMSO-d₆) δ 9.96 (d, J=7.6 Hz, 1H), 9.35 (dd, J=5.0, 1.7 Hz, 1H), 8.87 (d, J=4.2 Hz, 1H), 7.87 (dd, J=8.6, 5.0 Hz, 1H), 7.78 (dd, J=8.6, 1.7 Hz, 1H), 7.70 (d, J=4.2 Hz, 1H), 7.36-7.29 (m, 2H), 7.28-7.20 (m, 2H), 4.90-4.81 (m, 1H), 3.36 (dd, J=16.2, 6.9 Hz, 2H), 3.02 (dd, J=16.0, 4.0 Hz, 2H)

LCMS (Method 5): 1.04 min, 435.0, 437.0 [M+H]⁺

### Synthesis of compound 22, N7-indan-2-yl-2-pyridazin-3-yl-pyrazolo[1,5-a]pyrimidine-3,7-dicarboxamide

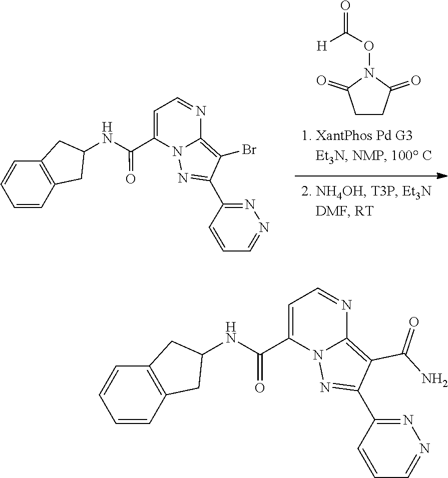 [SMILES: O=C(NC1Cc2ccccc2C1)c1ccnc2c(Br)c(-c3cccnn3)nn12 ; O=CON1C(=O)CCC1=O ; NC(=O)c1c(-c2cccnn2)nn2c(C(=O)NC3Cc4ccccc4C3)ccnc12]

3-Bromo-N-indan-2-yl-2-pyridazin-3-yl-pyrazolo[1,5-a]pyrimidine-7-carboxamide (50 mg, 0.11 mmol) and Et₃N (48 μL, 0.34 mmol) were dissolved in NMP (1 mL) and the solution was degassed by sparging with a stream of argon for 5 min. XantPhos Pd G3 (11 mg, 0.01 mmol) was added, and the solution was degassed with argon for a further 5 min. (2,5-Dioxopyrrolidin-1-yl) formate (82 mg, 0.57 mmol) was added and the vial was sealed immediately. The reaction was heated at 100° C. in a microwave reactor for 2 h then cooled to room temperature. Et₃N (48 μL, 0.34 mmol), 1-propanephosphonic anhydride (50 wt % in DMF; 137 μL, 0.23 mmol), and NH₄OH (243 μL, 1.15 mmol) were added sequentially and the reaction was stirred at room temperature for 2 h. The reaction mixture was diluted with EtOAc, filtered through a short pad of Dicalite®, and washed with EtOAc and water. The layers were separated, and the organic layer was washed with brine, dried through a hydrophobic frit, and concentrated in vacuo. The crude product was purified by preparative HPLC (acidic method) to provide the title compound (19 mg).

¹H NMR (400 MHz, DMSO-d₆) δ 10.00 (d, J=7.2 Hz, 1H), 9.32 (dd, J=5.0, 1.7 Hz, 1H), 8.96 (d, J=4.3 Hz, 1H), 7.91 (dd, J=8.5, 1.7 Hz, 1H), 7.85 (s, 1H), 7.83 (dd, J=8.5, 5.0 Hz, 1H), 7.70 (d, J=4.3 Hz, 1H), 7.61 (s, 1H), 7.30-7.22 (m, 2H), 7.21-7.12 (m, 2H), 4.86-4.73 (m, 1H), 3.41-3.33 (m, 2H), 2.96 (dd, J=16.0, 5.0 Hz, 2H)

LCMS (Method 4): 1.70 min, 400.0 [M+H]⁺

### Synthesis of 2-[2-(dimethylamino)ethylamino]-N-indan-2-yl-pyrazolo[1,5-a]pyrimidine-7-carboxamide

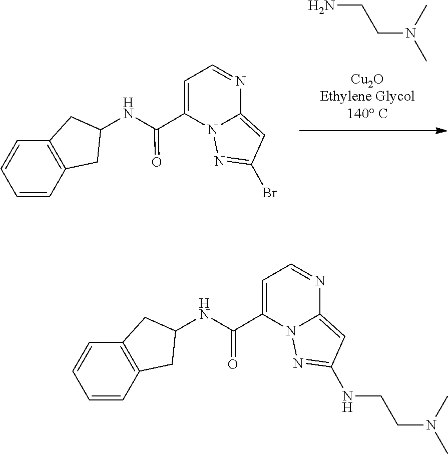

2-Bromo-N-indan-2-yl-pyrazolo[1,5-a]pyrimidine-7-carboxamide (200 mg, 0.56 mmol), Cu₂O (8 mg, 0.06 mmol) and N′,N′-dimethylethane-1,2-diamine (1.89 mL, 16.80 mmol) were suspended in ethylene glycol (2.2 mL) and the reaction was heated at 140° C. in a microwave reactor for 5 h. The reaction mixture was cooled to room temperature and diluted with water and EtOAc. The layers were separated, and the aqueous layer was extracted with EtOAc (×2). The combined organic layers were dried over Na₂SO₄ and concentrated in vacuo. The crude product was purified by column chromatography (eluting with 0 to 30% MeOH in DCM), and the resulting material was dissolved in DCM and washed with sat. aq. NaHCO₃. The organic layer was dried over Na₂SO₄ and concentrated in vacuo to provide the title compound (76 mg).

¹H NMR (400 MHZ, CDCl₃) δ 11.02 (d, J=7.9 Hz, 1H), 8.39 (d, J=4.5 Hz, 1H), 7.47 (d, J=4.5 Hz, 1H), 7.30-7.16 (m, 4H), 5.89 (s, 1H), 5.08-4.96 (m, 1H), 4.85 (t, J=5.1 Hz, 1H), 3.44 (dd, J=16.1, 6.8 Hz, 2H), 3.02 (dd, J=16.0, 4.0 Hz, 2H), 2.97-2.92 (m, 2H), 2.50-2.41 (m, 2H), 2.24 (s, 6H)

LCMS (Method 1): 1.25 min, 365.2 [M+H]⁺

### Synthesis of 3-bromo-2-[2-(dimethylamino)ethylamino]-N-indan-2-yl-pyrazolo[1,5-a]pyrimidine-7-carboxamide

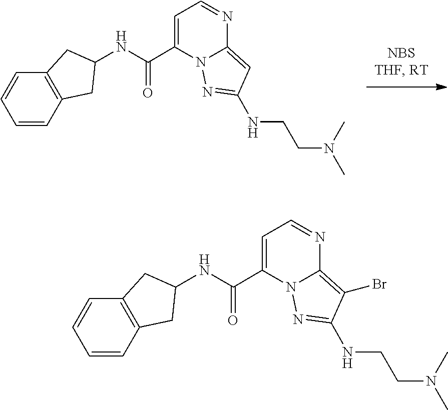 [SMILES: CN(C)CCNc1cc2nccc(C(=O)NC3Cc4ccccc4C3)n2n1 ; CN(C)CCNc1nn2c(C(=O)NC3Cc4ccccc4C3)ccnc2c1Br]

NBS (40 mg, 0.23 mmol) was added to a solution of 2-[2-(dimethylamino)ethylamino]-N-indan-2-yl-pyrazolo[1,5-a]pyrimidine-7-carboxamide (75 mg, 0.21 mmol) in THF (4 mL) and the reaction was stirred at room temperature for 1.5 h. The reaction mixture was diluted with DCM and washed with water. The aqueous layer was extracted with DCM, and the combined organic layers were dried over Na₂SO₄ and concentrated in vacuo to provide the title compound (94 mg).

¹H NMR (400 MHZ, CDCl₃) δ 10.81 (s, 1H), 8.49 (d, J=4.4 Hz, 1H), 7.52 (d, J=4.4 Hz, 1H), 7.29 (dd, J=5.4, 3.3 Hz, 2H), 7.22-7.20 (m, 2H), 5.06-5.01 (m, 1H), 3.46-3.40 (m, 2H), 3.00 (dd, J=16.1, 3.1 Hz, 2H), 2.91 (s, 2H), 2.47 (t, J=5.7 Hz, 2H), 1.25 (s, 6H). Exchangeable proton not observed.

LCMS (short acidic): 1.30 min, 443.2, 445.2 [M+H]⁺

### Synthesis of Compound 23, 2-[2-(dimethylamino)ethylamino]-N7-indan-2-yl-pyrazolo[1,5-a]pyrimidine-3,7-dicarboxamide

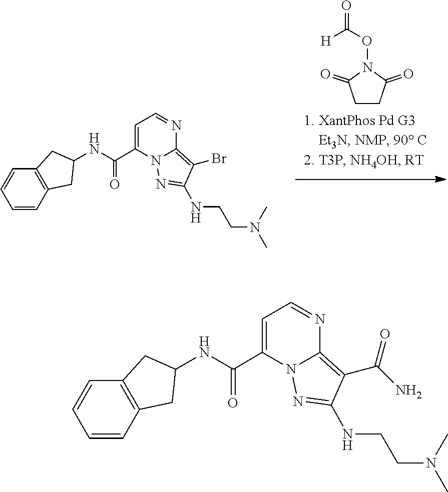 [SMILES: O=CON1C(=O)CCC1=O ; CN(C)CCNc1nn2c(C(=O)NC3Cc4ccccc4C3)ccnc2c1Br ; CN(C)CCNc1nn2c(C(=O)NC3Cc4ccccc4C3)ccnc2c1C(N)=O]

3-Bromo-2-[2-(dimethylamino)ethylamino]-N-indan-2-yl-pyrazolo[1,5-a]pyrimidine-7-carboxamide (91 mg, 0.21 mmol) and XantPhos Pd G3 (10 mg, 0.01 mmol) were placed in a vial and the vial was sealed and purged with argon for 5 min. NMP (2 mL) and Et₃N (86 μL, 0.62 mmol) were added and the solution was degassed by sparging with a stream of argon for 5 min. (2,5-Dioxopyrrolidin-1-yl) formate (147 mg, 1.03 mmol) was added and the vial was sealed immediately. The reaction was heated at 90° C. in a microwave reactor for 2 h. Additional portions of XantPhos Pd G3 (10 mg, 0.01 mmol) and Et₃N (86 UL, 0.62 mmol) were added and the mixture was purged with argon for 5 min. An additional portion of (2,5-dioxopyrrolidin-1-yl) formate (147 mg, 1.03 mmol) was added and the vial was sealed immediately. The reaction was heated at 90° C. in a microwave reactor for a further 3 h. The reaction mixture was cooled to room temperature, and NH₄OH (82 μL, 2.05 mmol) and 1-propanephosphonic anhydride (50 wt % in EtOAc; 240 μL, 0.41 mmol) were added. The reaction was stirred at room temperature for 3 h, then quenched with water and diluted with EtOAc. The layers were separated, and the aqueous layer was extracted with EtOAc (×2). The combined organic layers were washed with brine, dried through a hydrophobic frit, and concentrated in vacuo. The crude product was purified by preparative HPLC (acidic and basic methods) to provide the title compound (12 mg).

¹H NMR (400 MHz, DMSO-d₆) δ 10.56 (d, J=7.6 Hz, 1H), 8.64 (d, J=4.6 Hz, 1H), 7.50 (d, J=4.6 Hz, 1H), 7.39-7.27 (m, 4H), 7.25-7.16 (m, 2H), 7.03 (t, J=5.4 Hz, 1H), 4.88-4.77 (m, 1H), 3.37 (dd, J=16.2, 6.6 Hz, 2H), 3.04-2.92 (m, 4H), 2.40 (t, J=6.0 Hz, 2H), 2.16 (s, 6H)

LCMS (Method 2): 1.64 min, 408.2 [M+H]⁺

### Synthesis of ethyl 2-(1-tert-butoxycarbonylazetidin-3-yl)pyrazolo[1,5-a]pyrimidine-7-carboxylate

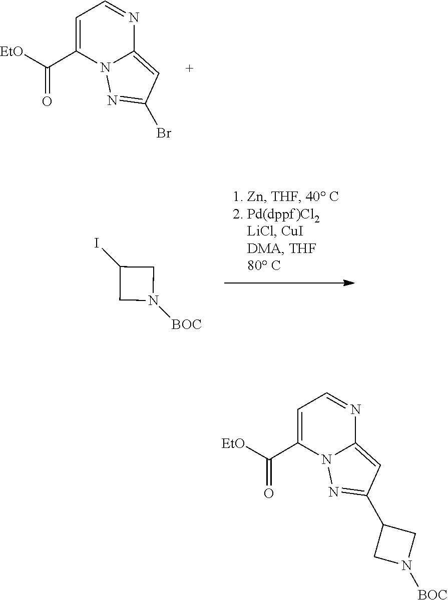 [SMILES: CCOC(=O)c1ccnc2cc(Br)nn12 ; CC(C)(C)OC(=O)N1CC(I)C1 ; CCOC(=O)c1ccnc2cc(C3CN(C(=O)OC(C)(C)C)C3)nn12]

A solution of tert-butyl 3-iodoazetidine-1-carboxylate (1.5 g, 5.30 mmol) in THF (8 mL) was pumped through a column packed with 12 g of zinc granules (flow reactor, 0.5 mL/min, zinc previously activated with TMSCl and dibromoethane) at 40° C. The resulting solution was collected to provide 10 mL of (1-tert-butoxycarbonylazetidin-3-yl)-iodo-zinc (~0.26 M in THF). Ethyl 2-bromopyrazolo[1,5-a]pyrimidine-7-carboxylate (500 mg, 1.85 mmol), Pd(dppf)Cl₂ (135 mg, 0.19 mmol), CuI (71 mg, 0.37 mmol), and LiCl (118 mg, 2.78 mmol) were placed in a vial and the vial was purged with argon for 15 min. DMA (7 mL) was added, followed by a solution of (1-tert-butoxycarbonylazetidin-3-yl)-iodo-zinc (~0.26 M in THF; 7.12 mL, 1.85 mmol), and the reaction was heated at 80° C. for 4 h. The reaction mixture was cooled to room temperature, diluted with EtOAc, and washed with sat. aq. NaHCO₃. The aqueous layer was extracted with EtOAc, and the combined organic layers were washed with brine (×6), dried over Na₂SO₄, and concentrated in vacuo. The crude product was purified by column chromatography (eluting with 0 to 100% EtOAc in heptane) to provide the title compound (167 mg).

¹H NMR (400 MHZ, CDCl₃) δ 8.54 (d, J=4.2 Hz, 1H), 7.27 (s, 1H), 6.84 (s, 1H), 4.56 (q, J=7.2 Hz, 2H), 4.39 (t, J=8.5 Hz, 2H), 4.20-4.02 (m, 3H), 1.49-1.44 (m, 3H), 1.47 (s, 9H)

LCMS (Method 1): 1.72 min, 247.2 [M-Boc+H]⁺

### Synthesis of 2-(1-tert-butoxycarbonylazetidin-3-yl)pyrazolo[1,5-a]pyrimidine-7-carboxylic Acid

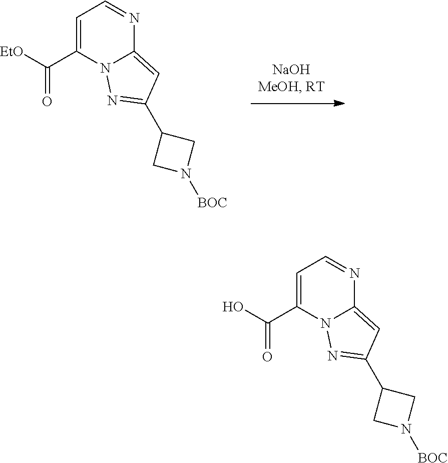 [SMILES: CCOC(=O)c1ccnc2cc(C3CN(C(=O)OC(C)(C)C)C3)nn12 ; CC(C)(C)OC(=O)N1CC(c2cc3nccc(C(=O)O)n3n2)C1]

NaOH (20 mg, 0.51 mmol) was added to a solution of ethyl 2-(1-tert-butoxycarbonylazetidin-3-yl)pyrazolo[1,5-a]pyrimidine-7-carboxylate (167 mg, 0.46 mmol) in MeOH (2 mL) and the reaction was stirred at room temperature for 4 h. The reaction mixture was acidified with 1 M aq. HCl and diluted with EtOAc and water. The layers were separated, and the aqueous layer was extracted with EtOAc. The combined organic layers were washed with brine, dried over Na₂SO₄, and concentrated in vacuo to provide the title compound (133 mg).

¹H NMR (400 MHZ, CDCl₃) δ 8.75 (d, J=4.2 Hz, 1H), 7.75 (d, J=4.2 Hz, 1H), 6.89 (s, 1H), 4.44 (t, J=8.7 Hz, 2H), 4.22-4.07 (m, 2H), 4.07-4.00 (m, 1H), 1.48 (s, 9H). Exchangeable proton not observed.

LCMS (Method 1): 1.57 min, 263.2 [M-t-Bu+H]⁺

### Synthesis of tert-butyl 3-[7-(indan-2-ylcarbamoyl)pyrazolo[1,5-a]pyrimidin-2-yl]azetidine-1-carboxylate

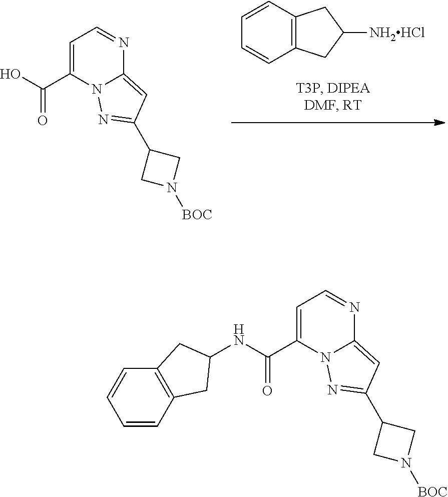

1-Propanephosphonic anhydride (50 wt % in EtOAc; 360 μL, 0.60 mmol) was added to a solution of 2-(1-tert-butoxycarbonylazetidin-3-yl)pyrazolo[1,5-a]pyrimidine-7-carboxylic acid (133 mg, 0.40 mmol), indan-2-amine hydrochloride (82 mg, 0.48 mmol), and DIPEA (210 μL, 1.20 mmol) in DMF (2 mL) and the reaction was stirred at room temperature for 19 h. The reaction mixture was diluted with EtOAc and washed with sat. aq. NaHCO₃. The aqueous layer was extracted with EtOAc, and the combined organic layers were washed with brine (×4), dried over Na₂SO₄, and concentrated in vacuo. The crude product was purified by column chromatography (eluting with 0 to 50% EtOAc in heptane) to provide the title compound (119 mg).

¹H NMR (400 MHZ, CDCl₃) δ 10.83 (d, J=7.1 Hz, 1H), 8.64 (d, J=4.3 Hz, 1H), 7.78 (d, J=4.3 Hz, 1H), 7.30 (dd, J=5.4, 3.3 Hz, 2H), 7.24-7.20 (m, 2H), 6.72 (d, J=0.5 Hz, 1H), 5.02-4.90 (m, 1H), 4.31 (t, J=8.7 Hz, 2H), 4.04 (dd, J=8.5, 6.0 Hz, 2H), 3.95-3.83 (m, 1H), 3.47 (dd, J=16.0, 6.9 Hz, 2H), 3.06 (dd, J=15.9, 4.8 Hz, 2H), 1.47 (s, 9H)

LCMS (Method 1): 1.94 min, 378.2 [M-t-Bu+H]⁺

### Synthesis of tert-butyl 3-[3-bromo-7-(indan-2-ylcarbamoyl)pyrazolo[1,5-a]pyrimidin-2-yl]azetidine-1-carboxylate

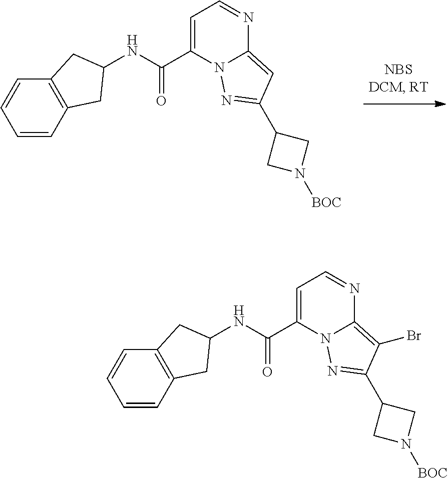 [SMILES: CC(C)(C)OC(=O)N1CC(c2cc3nccc(C(=O)NC4Cc5ccccc5C4)n3n2)C1 ; CC(C)(C)OC(=O)N1CC(c2nn3c(C(=O)NC4Cc5ccccc5C4)ccnc3c2Br)C1]

NBS (50 mg, 0.28 mmol) was added to a solution of tert-butyl 3-[7-(indan-2-ylcarbamoyl)pyrazolo[1,5-a]pyrimidin-2-yl]azetidine-1-carboxylate (119 mg, 0.27 mmol) in DCM (4 mL) and the reaction was stirred at room temperature for 87 h. The reaction mixture was diluted with DCM and washed with sat. aq. NaHCO₃. The aqueous layer was extracted with DCM, and the combined organic layers were washed with brine, dried over Na₂SO₄, and concentrated in vacuo to provide the title compound (133 mg).

¹H NMR (400 MHZ, CDCl₃) δ 10.55 (d, J=6.9 Hz, 1H), 8.73 (d, J=4.3 Hz, 1H), 7.85 (d, J=4.3 Hz, 1H), 7.35-7.29 (m, 2H), 7.27-7.19 (m, 2H), 5.02-4.90 (m, 1H), 4.33 (t, J=8.6 Hz, 2H), 4.16-4.09 (m, 2H), 4.09-3.98 (m, 1H), 3.48 (dd, J=16.0, 6.7 Hz, 2H), 3.07 (dd, J=16.0, 4.4 Hz, 2H), 1.49 (s, 9H)

LCMS (Method 1): 2.03 min, 412.0, 414.0 [M-Boc+H]⁺, 456.0, 458.0 [M-t-Bu+H]⁺

### Synthesis of tert-butyl 3-[3-carbamoyl-7-(indan-2-ylcarbamoyl)pyrazolo[1,5-a]pyrimidin-2-yl]azetidine-1-carboxylate

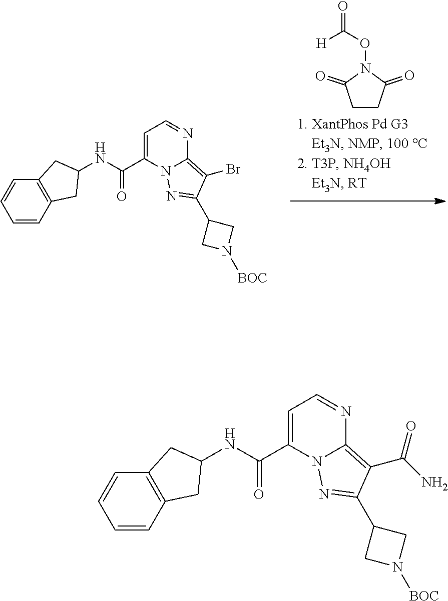 [SMILES: O=CON1C(=O)CCC1=O ; CC(C)(C)OC(=O)N1CC(c2nn3c(C(=O)NC4Cc5ccccc5C4)ccnc3c2Br)C1 ; CC(C)(C)OC(=O)N1CC(c2nn3c(C(=O)NC4Cc5ccccc5C4)ccnc3c2C(N)=O)C1]

XantPhos Pd G3 (24 mg, 0.02 mmol) was placed in a vial and the vial was sealed and purged with argon for 10 min. A separate sealed flask containing tert-butyl 3-[3-bromo-7-(indan-2-ylcarbamoyl)pyrazolo[1,5-a]pyrimidin-2-yl]azetidine-1-carboxylate (133 mg, 0.25 mmol) was purged with argon for 10 min. NMP (3 mL) was added to the flask, and the solution was transferred to the vial containing XantPhos Pd G3. Et₃N (70 μL, 0.50 mmol) was added, and the vial was purged with argon for a further 5 min. (2,5-Dioxopyrrolidin-1-yl) formate (178 mg, 1.25 mmol) was added, and the reaction was heated at 100° C. in a microwave reactor for 6 h. Additional portions of XantPhos Pd G3 (62 mg, 0.04 mmol) and (2,5-dioxopyrrolidin-1-yl) formate (356 mg, 2.50 mmol) were added, and the reaction was heated at 100° C. in a microwave reactor for a further 6 h. 1-Propanephosphonic anhydride (50 wt % in EtOAc; 220 μL, 0.37 mmol), Et₃N (100 μL, 0.75 mmol), and NH₄OH (170 μL, 2.49 mmol) were added, and the reaction was stirred at room temperature for 3 h. The reaction mixture was diluted with EtOAc and washed with sat. aq. NaHCO₃. The aqueous layer was extracted with EtOAc, and the combined organic layers were washed with brine (×4), dried over Na₂SO₄, and concentrated in vacuo. The crude product was purified by column chromatography (eluting with 0 to 100% EtOAc in heptane) to provide the title compound (46 mg).

¹H NMR (400 MHZ, CDCl₃) δ 10.60 (d, J=6.8 Hz, 1H), 8.79 (d, J=4.4 Hz, 1H), 8.00 (s, 1H), 7.95 (d, J=4.4 Hz, 1H), 7.32 (dd, J=5.4, 3.3 Hz, 2H), 7.22 (dd, J=5.5, 3.2 Hz, 2H), 5.56 (s, 1H), 5.00-4.89 (m, 1H), 4.56 (tt, J=8.8, 6.1 Hz, 1H), 4.36 (t, J=8.8 Hz, 2H), 4.06 (dd, J=8.8, 6.1 Hz, 2H), 3.48 (dd, J=16.0, 6.7 Hz, 2H), 3.08 (dd, J=16.0, 4.3 Hz, 2H), 1.46 (s, 9H)

LCMS (Method 1): 1.80 min, 421.2 [M-t-Bu+H]⁺

### Synthesis of 2-(azetidin-3-yl)-N7-indan-2-yl-pyrazolo[1,5-a]pyrimidine-3,7-dicarboxamide

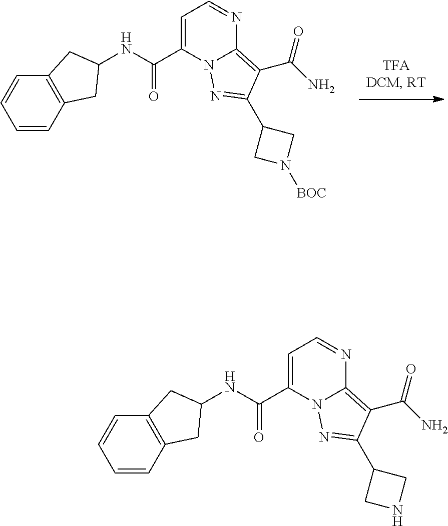 [SMILES: CC(C)(C)OC(=O)N1CC(c2nn3c(C(=O)NC4Cc5ccccc5C4)ccnc3c2C(N)=O)C1 ; NC(=O)c1c(C2CNC2)nn2c(C(=O)NC3Cc4ccccc4C3)ccnc12]

TFA (140 μL, 1.83 mmol) was added to a solution of tert-butyl 3-[3-carbamoyl-7-(indan-2-ylcarbamoyl)pyrazolo[1,5-a]pyrimidin-2-yl]azetidine-1-carboxylate (46 mg, 0.09 mmol) in DCM (2 mL) and the reaction was stirred at room temperature for 2 h then concentrated in vacuo. The crude product was purified by SCX chromatography (eluting with 2 M NH₃ in MeOH) to provide the title compound (28 mg).

¹H NMR (400 MHZ, CDCl₃) δ 10.74 (d, J=7.7 Hz, 1H), 8.77 (d, J=4.4 Hz, 1H), 8.04 (s, 1H), 7.94 (d, J=4.4 Hz, 1H), 7.32 (dd, J=5.4, 3.3 Hz, 2H), 7.25-7.22 (m, 2H), 5.65 (s, 1H), 5.10-4.99 (m, 1H), 4.76-4.64 (m, 1H), 4.00 (t, J=8.4 Hz, 2H), 3.77-3.65 (m, 2H), 3.52-3.42 (m, 2H), 3.07 (dd, J=16.1, 3.5 Hz, 2H). Additional exchangeable proton not observed.

LCMS (Method 1): 1.21 min, 377.2 [M+H]⁺

### Synthesis of compound 24, N7-indan-2-yl-2-(1-methylazetidin-3-yl)pyrazolo[1,5-a]pyrimidine-3,7-dicarboxamide

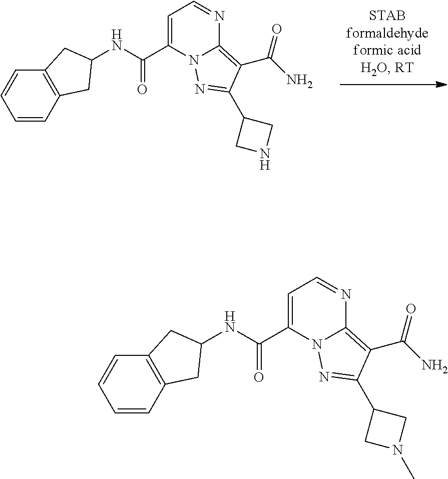 [SMILES: NC(=O)c1c(C2CNC2)nn2c(C(=O)NC3Cc4ccccc4C3)ccnc12 ; CN1CC(c2nn3c(C(=O)NC4Cc5ccccc5C4)ccnc3c2C(N)=O)C1]

Formaldehyde (37 wt % in water; 40 μL, 0.53 mmol) was added to a suspension of 2-(azetidin-3-yl)-N7-indan-2-yl-pyrazolo[1,5-a]pyrimidine-3,7-dicarboxamide (44 mg, 0.11 mmol) in water (2 mL) and the reaction was stirred at room temperature for 5 min. Formic acid (20 μL, 0.53 mmol) was added, and the reaction was stirred for a further 1 h. Sodium triacetoxyborohydride (112 mg, 0.53 mmol) was added, and the reaction was stirred for a further 1 h, then diluted with EtOAc and sat. aq. NaHCO₃. The layers were separated, and the aqueous layer was extracted with EtOAc. The combined organic layers were washed with brine (×2), dried over Na₂SO₄, and concentrated in vacuo. The crude product was purified by preparative HPLC (basic method) to provide the title compound (19 mg).

¹H NMR (400 MHz, DMSO-d₆) δ 10.38 (d, J=7.4 Hz, 1H), 8.89 (d, J=4.4 Hz, 1H), 7.73 (d, J=4.4 Hz, 1H), 7.63 (s, 1H), 7.47 (s, 1H), 7.35-7.27 (m, 2H), 7.24-7.16 (m, 2H), 4.90-4.80 (m, 1H), 4.28-4.16 (m, 1H), 3.56-3.47 (m, 2H), 3.45-3.37 (m, 2H), 3.04-2.90 (m, 4H), 2.16 (s, 3H)

LCMS (Method 2): 1.49 min, 391.2 [M+H]⁺

### Synthesis of 2-bromo-N-(3,3-difluorocyclobutyl)pyrazolo[1,5-a]pyrimidine-7-carboxamide

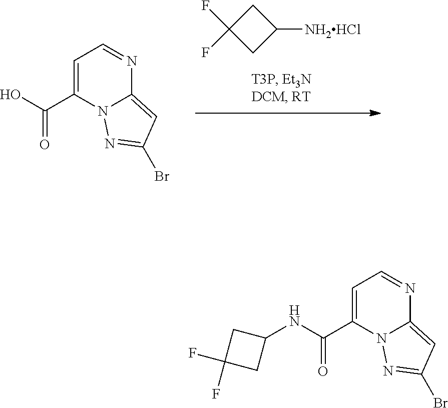

3-Bromopyrazolo[1,5-a]pyrimidine-7-carboxylic acid (500 mg, 2.07 mmol), 3,3-difluorocyclobutanamine hydrochloride (326 mg, 2.27 mmol), Et₃N (860 μL, 6.20 mmol), and 1-propanephosphonic anhydride (50 wt % in EtOAc; 2.27 mL, 2.27 mmol) were dissolved in DCM (10 mL) and the reaction was stirred at room temperature overnight. The reaction mixture was concentrated in vacuo, and the crude product was purified by column chromatography (eluting with 0 to 100% EtOAc in heptane) to provide the title compound (523 mg).

LCMS (Method 5): 1.06 min, 331.0, 333.0 [M+H]⁺

### Synthesis of N-(3,3-difluorocyclobutyl)-2-(4,4,5,5-tetramethyl-1,3,2-dioxaborolan-2-yl)pyrazolo[1,5-a]pyrimidine-7-carboxamide

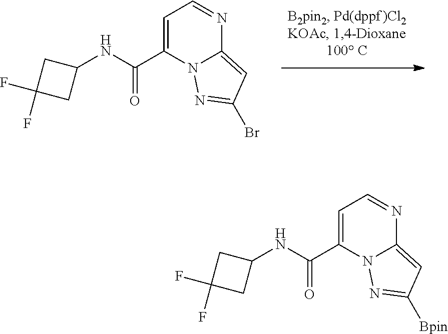

2-Bromo-N-(3,3-difluorocyclobutyl)pyrazolo[1,5-a]pyrimidine-7-carboxamide (523 mg, 1.58 mmol), B₂pin₂ (481 mg, 1.90 mmol), and KOAc (465 mg, 4.74 mmol) were suspended in 1,4-dioxane (10 mL) and the mixture was degassed by sparging with a stream of argon for 10 min. Pd(dppf)Cl₂ (115 mg, 0.16 mmol) was added, and the mixture was sparged with argon for a further 10 min. The reaction was heated at 100° C. for 16 h then cooled to room temperature. The reaction mixture was filtered through a short pad of Dicalite®, washed with EtOAc, and concentrated in vacuo. The crude product was purified by column chromatography (eluting with 0 to 100% EtOAc in heptane) to provide the title compound (427 mg).

¹H NMR (400 MHz, DMSO-d₆) δ 10.33 (d, J=6.3 Hz, 1H), 8.73 (d, J=4.2 Hz, 1H), 7.49 (d, J=4.1 Hz, 1H), 7.13 (s, 1H), 4.41-4.28 (m, 1H), 3.20-3.05 (m, 2H), 2.82-2.68 (m, 2H), 1.16 (s, 12H)

LCMS (Method 5): 0.69 min, 297.2 [M+H]⁺ (boronic acid)

### Synthesis of N-(3,3-difluorocyclobutyl)-2-oxazol-2-yl-pyrazolo[1,5-a]pyrimidine-7-carboxamide

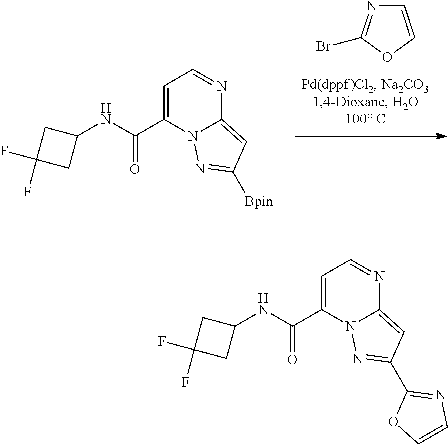

N-(3,3-difluorocyclobutyl)-2-(4,4,5,5-tetramethyl-1,3,2-dioxaborolan-2-yl)pyrazolo[1,5-a]pyrimidine-7-carboxamide (163 mg, 0.43 mmol), 2-bromooxazole (67 mg, 0.45 mmol), and Na₂CO₃ (137 mg, 1.29 mmol), were suspended in 1,4-dioxane (4 mL) and water (1 mL) and the mixture was degassed by sparging with a stream of argon for 5 min. Pd(dppf)Cl₂ (32 mg, 0.04 mmol) was added, and the reaction was heated at 100° C. overnight. The reaction mixture was cooled to room temperature and diluted with EtOAc and water. The layers were separated, and the aqueous layer was extracted with EtOAc. The combined organic layers were washed with brine, dried over Na₂SO₄, and concentrated in vacuo. The crude product was purified by column chromatography (eluting with 0 to 100% EtOAc in heptane) to provide the title compound (76 mg).

¹H NMR (400 MHz, DMSO-d₆) δ 10.07 (d, J=6.4 Hz, 1H), 8.80 (d, J=4.2 Hz, 1H), 8.37 (d, J=0.8 Hz, 1H), 7.55 (d, J=4.2 Hz, 1H), 7.52 (d, J=0.8 Hz, 1H), 7.41 (s, 1H), 4.39 (dd, J=8.3, 6.6 Hz, 1H), 3.21-3.07 (m, 2H), 2.85-2.68 (m, 2H)

LCMS (Method 5): 0.90 min, 320.2 [M+H]⁺

### Synthesis of 3-bromo-N-(3,3-difluorocyclobutyl)-2-oxazol-2-yl-pyrazolo[1,5-a]pyrimidine-7-carboxamide

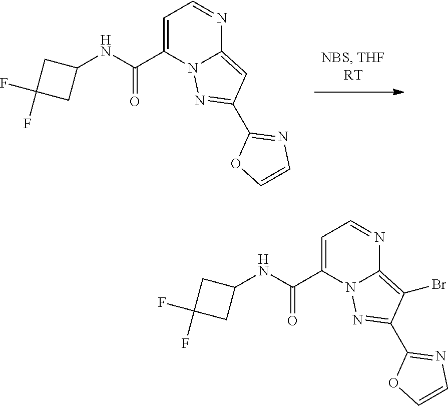 [SMILES: O=C(NC1CC(F)(F)C1)c1ccnc2cc(-c3ncco3)nn12 ; O=C(NC1CC(F)(F)C1)c1ccnc2c(Br)c(-c3ncco3)nn12]

N-(3,3-difluorocyclobutyl)-2-oxazol-2-yl-pyrazolo[1,5-a]pyrimidine-7-carboxamide (108 mg, 0.34 mmol) and NBS (93 mg, 0.52 mmol) were dissolved in THF (5 mL) and the reaction was stirred at room temperature overnight. The reaction mixture was concentrated in vacuo and the crude product was purified by column chromatography (eluting with 0 to 100% EtOAc in heptane) to provide the title compound (91 mg).

¹H NMR (400 MHz, DMSO-d₆) δ 9.91 (d, J=6.4 Hz, 1H), 8.87 (d, J=4.2 Hz, 1H), 8.44 (d, J=0.8 Hz, 1H), 7.62 (d, J=4.2 Hz, 1H), 7.59 (d, J=0.8 Hz, 1H), 4.42-4.34 (m, 1H), 3.20-3.05 (m, 2H), 2.83-2.70 (m, 2H)

LCMS (Method 5): 1.00 min, 398.0, 400.0 [M+H]⁺

### Synthesis of compound 25, N7-(3,3-difluorocyclobutyl)-2-oxazol-2-yl-pyrazolo[1,5-a]pyrimidine-3,7-dicarboxamide

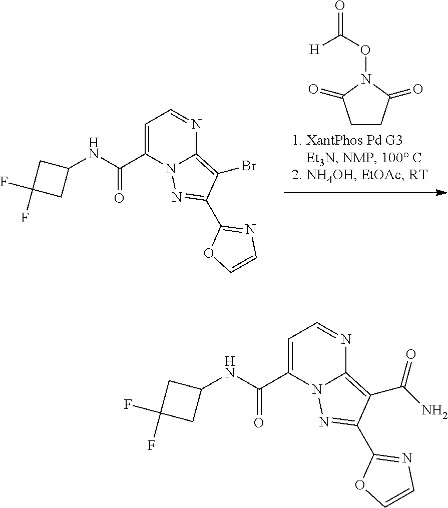 [SMILES: O=C(NC1CC(F)(F)C1)c1ccnc2c(Br)c(-c3ncco3)nn12 ; O=CON1C(=O)CCC1=O ; NC(=O)c1c(-c2ncco2)nn2c(C(=O)NC3CC(F)(F)C3)ccnc12]

3-Bromo-N-(3,3-difluorocyclobutyl)-2-oxazol-2-yl-pyrazolo[1,5-a]pyrimidine-7-carboxamide (91 mg, 0.23 mmol) and Et₃N (96 μL, 0.69 mmol) were dissolved in NMP (4 mL) and the solution was degassed by sparging with a stream of argon for 5 min. XantPhos Pd G3 (22 mg, 0.02 mmol) and (2,5-dioxopyrrolidin-1-yl) formate (164 mg, 1.14 mmol) were added, and the vial was sealed immediately. The reaction was heated at 100° C. in a microwave reactor for 2 h. Additional portions of XantPhos Pd G3 (11 mg, 0.01 mmol), Et₃N (50 μL, 0.34 mmol), and (2,5-dioxopyrrolidin-1-yl) formate (82 mg, 0.57 mmol) were added, and the solution was degassed by sparging with a stream of argon for 5 min. The reaction was heated at 100° C. in a microwave reactor for a further 2 h then cooled to room temperature. The reaction mixture was diluted with EtOAc, NH₄OH (320 μL, 2.29 mmol) was added, and the reaction was stirred at room temperature for 2 h. The reaction mixture was diluted with EtOAc and water and the layers were separated. The aqueous layer was extracted with EtOAc, and the combined organic layers were dried over Na₂SO₄ and concentrated in vacuo. The crude product was purified by column chromatography (eluting with 0 to 10% MeOH in DCM) and preparative HPLC (basic method) to provide the title compound (20 mg).

¹H NMR (400 MHz, DMSO-d₆) δ 9.90 (d, J=6.4 Hz, 1H), 8.96 (d, J=4.3 Hz, 1H), 8.38 (d, J=0.9 Hz, 1H), 7.98 (s, 1H), 7.65 (d, J=4.3 Hz, 2H), 7.52 (d, J=0.8 Hz, 1H), 4.43-4.31 (m, 1H), 3.18-3.02 (m, 2H), 2.83-2.70 (m, 2H)

LCMS (Method 4): 1.55 min, 363.0 [M+H]⁺

### Synthesis of methyl 3-aminopyridazine-4-carboxylate

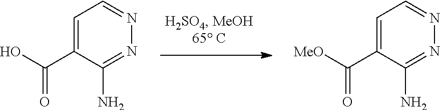 [SMILES: Nc1nnccc1C(=O)O ; COC(=O)c1ccnnc1N]

Concentrated H₂SO₄ (2 mL, 37.52 mmol) was added to a solution of 3-aminopyridazine-4-carboxylic acid (2 g, 14.38 mmol) in MeOH (40 mL) and the reaction was stirred at 65° C. for 1 week. The reaction mixture was cooled to room temperature and concentrated in vacuo. The residue was diluted with water and the solution was basified to pH 8 with NaHCO₃. The aqueous layer was extracted with EtOAc (×3), and the combined organic layers were dried over Na₂SO₄ and concentrated in vacuo to provide the title compound (1.85 g).

¹H NMR (400 MHz, DMSO-d₆) δ 8.65 (d, J=4.9 Hz, 1H), 7.72 (d, J=4.9 Hz, 1H), 7.28 (s, 2H), 3.87 (s, 3H)

LCMS (Method 1): 0.84 min, 154.2 [M+H]⁺

### Synthesis of methyl 2-methylimidazo[1,2-b]pyridazine-8-carboxylate

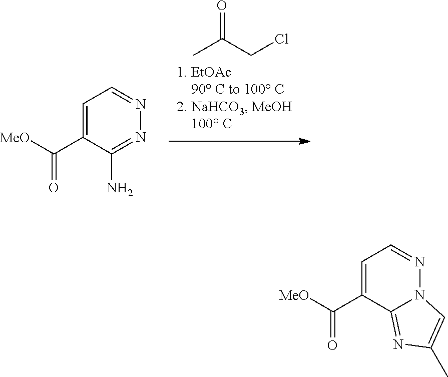

Methyl 3-aminopyridazine-4-carboxylate (740 mg, 4.83 mmol) was added to a solution of 1-chloropropan-2-one (3.85 mL, 48.32 mmol) in EtOAc (9 mL) and the reaction was heated at 90° C. for 16 h, and then at 100° C. in a microwave reactor for 5 h. MeOH (3 mL) and NaHCO₃ (812 mg, 9.66 mmol) were added, and the reaction was heated at 100° C. in a microwave reactor for 6 h. The reaction mixture was cooled to room temperature and concentrated in vacuo. The crude product was purified by column chromatography (eluting with 0 to 30% MeOH in DCM) to provide the title compound (355 mg).

¹H NMR (400 MHz, MeOD) δ 8.50 (d, J=4.7 Hz, 1H), 8.02 (q, J=0.9 Hz, 1H), 7.65 (d, J=4.7 Hz, 1H), 4.03 (s, 3H), 2.51 (d, J=0.9 Hz, 3H)

LCMS (Method 1): 0.96 min, 192.2 [M+H]⁺

### Synthesis of 2-methylimidazo[1,2-b]pyridazine-8-carboxylic Acid

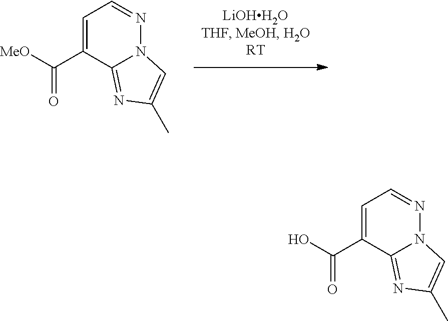

A solution of LiOH·H₂O (75 mg, 1.79 mmol) in water (1 mL) was added dropwise to a solution of methyl 2-methylimidazo[1,2-b]pyridazine-8-carboxylate (345 mg, 1.62 mmol) in THF (5 mL) and MeOH (1 mL) and the reaction was stirred at room temperature for 1 h. The reaction mixture was concentrated in vacuo and diluted with water. The aqueous solution was acidified with 1 M aq. HCl and extracted with a 3:1 mixture of CHCl₃:IPA. The aqueous layer was concentrated in vacuo to provide the title compound (361 mg). The crude product was taken forward to the next step without further purification.

¹H NMR (400 MHz, MeOD) δ 8.96 (d, J=4.8 Hz, 1H), 8.32 (s, 1H), 8.11 (d, J=4.9 Hz, 1H), 2.64 (s, 3H). Exchangeable proton not observed.

### Synthesis of N-(3,3-difluorocyclobutyl)-2-methyl-imidazo[1,2-b]pyridazine-8-carboxamide

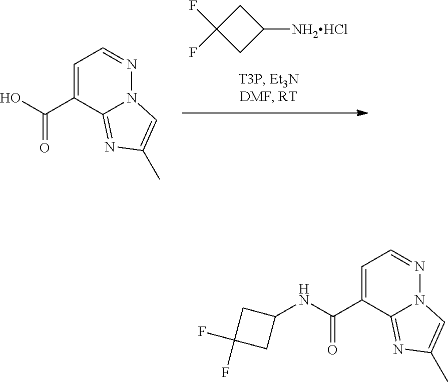

3,3-Difluorocyclobutanamine hydrochloride (281 mg, 1.96 mmol) was added to a solution of 2-methylimidazo[1,2-b]pyridazine-8-carboxylic acid (289 mg, 1.63 mmol), Et₃N (1.14 mL, 8.16 mmol), and 1-propanephosphonic anhydride (50 wt % in DMF; 1.94 mL, 3.26 mmol) in DMF (6 mL) and the reaction was stirred at room temperature for 20 h. The reaction mixture was diluted with EtOAc and brine and the layers were separated. The aqueous layer was extracted with EtOAc (×2), and the combined organic layers were washed with 1 M aq. HCl. The aqueous layer was basified to pH 8 with NaHCO₃ and extracted with EtOAc (×2). The combined organic layers were dried over Na₂SO₄ and concentrated in vacuo to provide the title compound (169 mg).

¹H NMR (400 MHZ, CDCl₃) δ 10.54 (s, 1H), 8.66 (d, J=4.8 Hz, 1H), 8.35 (d, J=4.8 Hz, 1H), 7.89 (q, J=0.8 Hz, 1H), 4.52-4.40 (m, 1H), 3.16-2.86 (m, 4H), 2.67 (d, J=0.9 Hz, 3H)

LCMS (Method 1): 1.59 min, 267.2 [M+H]⁺

### Synthesis of 3-bromo-N-(3,3-difluorocyclobutyl)-2-methyl-imidazo[1,2-b]pyridazine-8-carboxamide

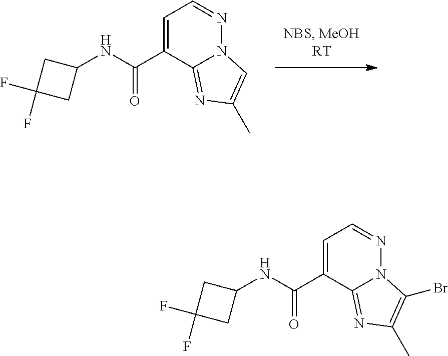 [SMILES: Cc1cn2nccc(C(=O)NC3CC(F)(F)C3)c2n1 ; Cc1nc2c(C(=O)NC3CC(F)(F)C3)ccnn2c1Br]

NBS (121 mg, 0.68 mmol) was added to a solution of N-(3,3-difluorocyclobutyl)-2-methyl-imidazo[1,2-b]pyridazine-8-carboxamide (165 mg, 0.62 mmol) in MeOH (5 mL) and the reaction was stirred at room temperature for 30 min then concentrated in vacuo. The residue was diluted with DCM and water, and the layers were separated. The aqueous layer was extracted with DCM, and the combined organic layers were dried over Na₂SO₄ and concentrated in vacuo to provide the title compound (182 mg). The crude product was taken forward to the next step without further purification.

¹H NMR (400 MHZ, CDCl₃) δ 10.13 (s, 1H), 8.59 (d, J=4.7 Hz, 1H), 7.89 (d, J=4.7 Hz, 1H), 4.54-4.46 (m, 1H), 3.17-3.09 (m, 2H), 2.83-2.71 (m, 2H), 2.56 (s, 3H)

LCMS (Method 1): 1.82 min, 345.0, 347.0 [M+H]⁺

### Synthesis of compound 26, N8-(3,3-difluorocyclobutyl)-2-methyl-imidazo[1,2-b]pyridazine-3,8-dicarboxamide

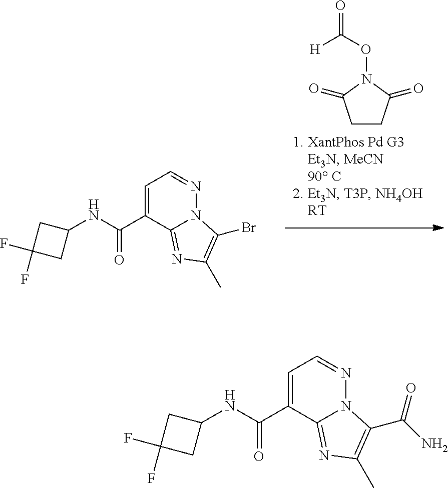 [SMILES: O=CON1C(=O)CCC1=O ; Cc1nc2c(C(=O)NC3CC(F)(F)C3)ccnn2c1Br ; Cc1nc2c(C(=O)NC3CC(F)(F)C3)ccnn2c1C(N)=O]

3-Bromo-N-(3,3-difluorocyclobutyl)-2-methyl-imidazo[1,2-b]pyridazine-8-carboxamide (180 mg, 0.52 mmol), XantPhos Pd G3 (50 mg, 0.05 mmol), and Et₃N (220 μL, 1.56 mmol) were dissolved in MeCN (5 mL) and the solution was degassed by sparging with a stream of argon for 30 min. (2,5-Dioxopyrrolidin-1-yl) formate (373 mg, 2.61 mmol) was added and the reaction vial was sealed immediately. The reaction was heated at 90° C. for 3.5 h then cooled to room temperature. Et₃N (220 μL, 1.56 mmol), 1-propanephosphonic anhydride (50 wt % in EtOAc; 620 μL, 1.04 mmol), and NH₄OH (620 μL, 5.22 mmol) were added and the reaction was stirred at room temperature for 16 h. The reaction mixture was concentrated in vacuo and the crude product was purified by column chromatography (eluting with 0 to 8% MeOH in DCM) followed by preparative HPLC (basic method) to provide the title compound (20 mg).

¹H NMR (400 MHz, DMSO-d₆) δ 10.00 (d, J=6.5 Hz, 1H), 8.87 (d, J=4.8 Hz, 1H), 8.18 (s, 1H), 7.97 (s, 1H), 7.85 (d, J=4.8 Hz, 1H), 4.40-4.33 (m, 1H), 3.16-3.03 (m, 2H), 2.88-2.76 (m, 2H), 2.75 (s, 3H)

LCMS (Method 2): 1.90 min, 310.2 [M+H]⁺

### Synthesis of 2-(2-isopropoxyacetyl)propanedinitrile

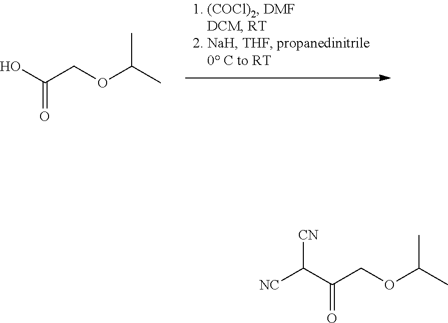

DMF (105 μL, 1.35 mmol) was added to a solution of 2-isopropoxyacetic acid (1.6 g, 13.54 mmol) in DCM (32 mL). Oxalyl chloride (1.39 mL, 16.25 mmol) was added dropwise over 1 h, and the reaction mixture was stirred at room temperature for 1 h then concentrated in vacuo to provide the crude acid chloride intermediate. A solution of propanedinitrile (895 mg, 13.55 mmol) in THF (12 mL) was added dropwise over 15 min to a suspension of NaH (1.08 g, 27.09 mmol) in THF (12 mL) at 0° C. The mixture was stirred at room temperature for 15 min then cooled back to 0° C. A solution of the acid chloride in THF (12 mL) was added dropwise over 15 min, then the reaction was stirred at room temperature for 17 h. The reaction mixture was quenched with water, acidified with 1 M aq. HCl, and diluted with EtOAc. The layers were separated, and the aqueous layer was extracted with EtOAc (×2). The combined organic layers were washed with brine, dried over Na₂SO₄, and concentrated in vacuo. The crude product was purified by column chromatography (eluting with 0 to 5% MeOH in DCM) to provide the title compound (1.43 g).

¹H NMR (400 MHZ, CDCl₃) δ 4.45 (s, 2H), 3.86 (hept, J=6.1 Hz, 1H), 3.60 (s, 1H), 1.28 (d, J=6.1 Hz, 6H)

LCMS (Method 1): 1.37 min (product does not ionise)

### Synthesis of 2-(2-isopropoxy-1-methoxy-ethylidene)propanedinitrile

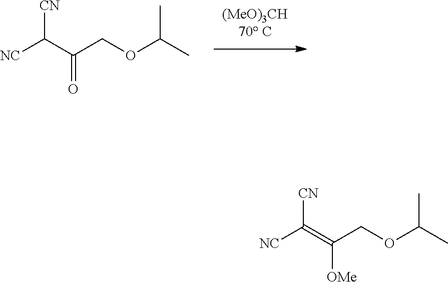

2-(1-Hydroxy-2-isopropoxy-ethylidene)propanedinitrile (1.43 g, 8.61 mmol) was dissolved in trimethoxymethane (15 mL, 137.11 mmol) and the reaction mixture was heated at 70° C. in a microwave reactor for 6 h, then cooled to room temperature and concentrated in vacuo.

The crude product was purified by column chromatography (eluting with 0 to 100% EtOAc in heptane) to provide the title compound (1.1 g).

¹H NMR (400 MHZ, CDCl₃) δ 4.41 (s, 2H), 4.23 (s, 3H), 3.73 (hept, J=6.0 Hz, 1H), 1.23 (d, J=6.1 Hz, 6H)

LCMS (Method 1): 1.63 min (product does not ionise)

### Synthesis of 5-amino-3-(isopropoxymethyl)-1H-pyrazole-4-carbonitrile

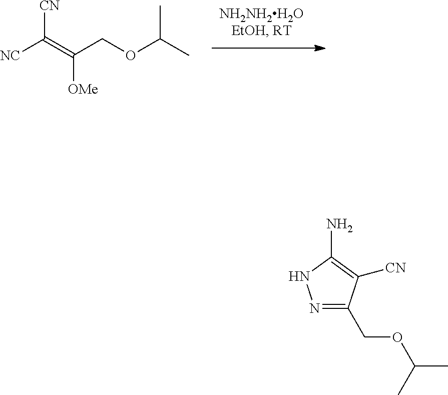

Hydrazine monohydrate (500 μL, 10.26 mmol) was added to a solution of 2-(2-isopropoxy-1-methoxy-ethylidene)propanedinitrile (1.12 g, 6.22 mmol) in EtOH (8.6 mL) and the reaction was stirred at room temperature for 1.5 h then concentrated in vacuo. The crude product was purified by column chromatography (eluting with 0 to 10% MeOH in DCM) to provide the title compound (921 mg).

¹H NMR (400 MHz, DMSO-d₆) δ 11.81 (s, 1H), 6.29 (s, 2H), 4.26 (s, 2H), 3.60 (hept, J=6.1 Hz, 1H), 1.10 (d, J=6.1 Hz, 6H)

LCMS (Method 1): 1.27 min, 181.2 [M+H]⁺

### Synthesis of ethyl 3-cyano-2-(isopropoxymethyl)pyrazolo[1,5-a]pyrimidine-7-carboxylate

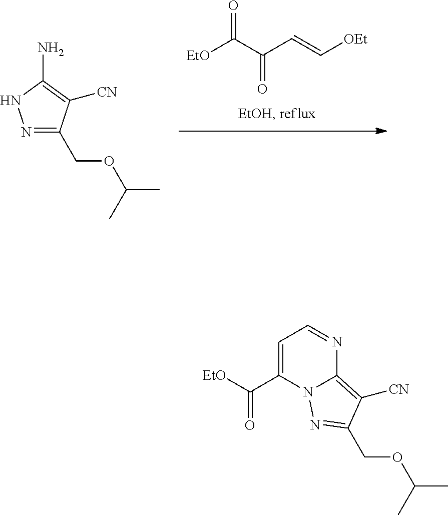 [SMILES: CC(C)OCc1n[nH]c(N)c1C#N ; CCO/C=C/C(=O)C(=O)OCC ; CCOC(=O)c1ccnc2c(C#N)c(COC(C)C)nn12]

5-Amino-3-(isopropoxymethyl)-1H-pyrazole-4-carbonitrile (921 mg, 5.11 mmol) and ethyl (E)-4-ethoxy-2-oxo-but-3-enoate (1.32 g, 7.67 mmol) were dissolved in EtOH (7 mL) and the reaction was heated at reflux for 2.5 h, then cooled to room temperature and concentrated in vacuo. The crude product was purified by column chromatography (eluting with 0 to 50% EtOAc in heptane) to provide the title compound (1.25 g) containing approximately 14% of the regioisomeric product as an impurity.

¹H NMR (400 MHZ, CDCl₃) δ 8.79 (d, J=4.2 Hz, 1H), 7.47 (d, J=4.2 Hz, 1H), 4.85 (s, 2H), 4.57 (q, J=7.1 Hz, 2H), 3.91-3.77 (m, 1H), 1.47 (t, J=7.2 Hz, 3H), 1.29 (d, J=6.1 Hz, 6H).

LCMS (Method 1): 1.70 min, 289.2 [M+H]⁺

### Synthesis of 3-cyano-2-(isopropoxymethyl)pyrazolo[1,5-a]pyrimidine-7-carboxylic Acid

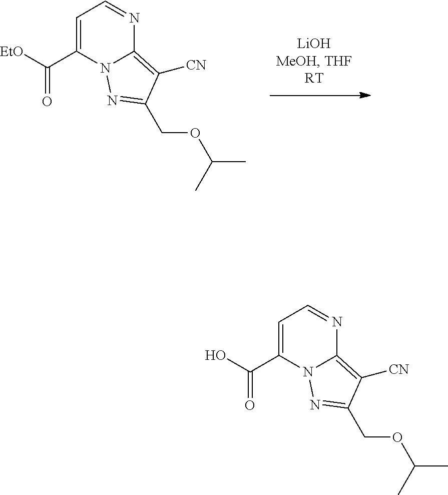 [SMILES: CCOC(=O)c1ccnc2c(C#N)c(COC(C)C)nn12 ; CC(C)OCc1nn2c(C(=O)O)ccnc2c1C#N]

4 M aq. LiOH (5.42 mL, 21.68 mmol) was added to a solution of ethyl 3-cyano-2-(isopropoxymethyl)pyrazolo[1,5-a]pyrimidine-7-carboxylate (1.25 g, 4.34 mmol) in THF (12.5 mL) and MeOH (4.2 mL) and the reaction was stirred at room temperature for 1 h then concentrated in vacuo. The residue was dissolved in water and the solution was acidified with 5 M aq. HCl and diluted with DCM. The layers were separated, and the aqueous layer was extracted with DCM (×2). The combined organic layers were dried through a hydrophobic frit and concentrated in vacuo. The crude product was purified by column chromatography (eluting with 0 to 5% MeOH in DCM) to provide the title compound (793 mg).

¹H NMR (400 MHZ, CDCl₃) δ 8.99 (d, J=6.1 Hz, 1H), 7.97 (d, J=4.3 Hz, 1H), 4.88 (s, 2H), 3.86 (hept, J=6.1 Hz, 1H), 1.31 (d, J=6.1 Hz, 6H). Exchangeable proton not observed.

LCMS (Method 1): 1.37 min, 261.2 [M+H]⁺

### Synthesis of 3-cyano-N-indan-2-yl-2-(isopropoxymethyl)pyrazolo[1,5-a]pyrimidine-7-carboxamide

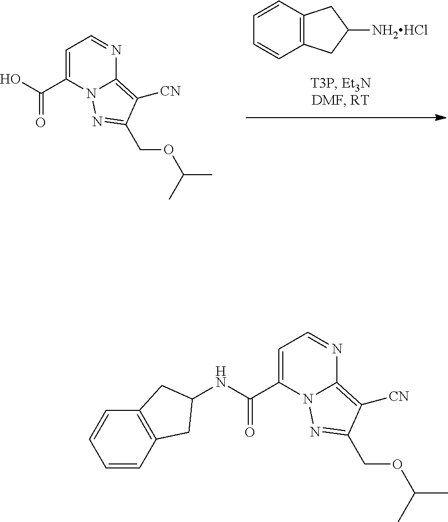

Et₃N (241 μL, 1.73 mmol) and 1-propanephosphonic anhydride (50 wt % in DMF; 1.03 mL, 1.73 mmol) were added to a solution of 3-cyano-2-(isopropoxymethyl)pyrazolo[1,5-a]pyrimidine-7-carboxylic acid (150 mg, 0.58 mmol) and indan-2-amine hydrochloride (108 mg, 0.63 mmol) in DMF (1.9 mL) and the reaction was stirred at room temperature for 20 h. The reaction was quenched with water and diluted with DCM. The layers were separated, and the aqueous layer was extracted with DCM (×2). The combined organic layers were dried through a hydrophobic frit and concentrated in vacuo. The crude product was purified by column chromatography (eluting with 0 to 50% EtOAc in heptane) to provide the title compound (178 mg).

¹H NMR (400 MHz, DMSO-d₆) δ 9.75 (d, J=7.1 Hz, 1H), 8.94 (d, J=4.4 Hz, 1H), 7.67 (d, J=4.4 Hz, 1H), 7.32-7.23 (m, 2H), 7.22-7.13 (m, 2H), 4.84-4.74 (m, 1H), 4.73 (s, 2H), 3.74 (hept, J=6.1 Hz, 1H), 3.37 (dd, J=16.2 Hz, 7.5 Hz 2H), 2.97 (dd, J=16.1, 5.2 Hz, 2H), 1.16 (d, J=6.1 Hz, 6H)

### Synthesis of compound 27, N7-indan-2-yl-2-(isopropoxymethyl)pyrazolo[1,5-a]pyrimidine-3,7-dicarboxamide

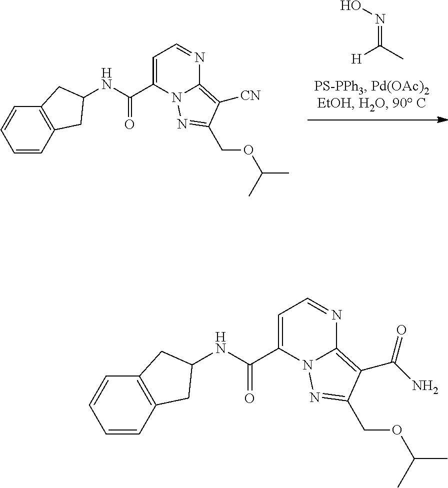

3-Cyano-N-indan-2-yl-2-(isopropoxymethyl)pyrazolo[1,5-a]pyrimidine-7-carboxamide (178 mg, 0.47 mmol), acetaldehyde oxime (116 μL, 1.9 mmol), Pd(OAc)₂ (16 mg, 0.07 mmol), and PS-PPh₃ (1.4-2 mmol/g; 49 mg, 0.07-0.1 mmol) were suspended in EtOH (3.6 mL) and water (900 μL) and the reaction was heated at 90° C. for 2 h. The reaction mixture was cooled to room temperature and diluted with DCM. The layers were separated, and the aqueous layer was extracted with DCM (×2). The combined organic layers were dried through a hydrophobic frit and concentrated in vacuo. The crude product was purified by column chromatography (eluting with 0 to 8% MeOH in DCM) to provide the title compound (141 mg).

¹H NMR (400 MHz, DMSO-d₆) δ 10.24 (d, J=6.8 Hz, 1H), 8.90 (d, J=4.4 Hz, 1H), 7.69 (d, J=4.4 Hz, 1H), 7.68 (br. s, 1H), 7.55 (br. s, 1H), 7.27 (dd, J=5.4, 3.3 Hz, 2H), 7.22-7.13 (m, 2H), 4.96 (s, 2H), 4.85-4.73 (m, 1H), 3.74 (hept, J=6.1 Hz, 1H), 3.41 (dd, J=7.5 Hz, 2H), 2.98 (dd, J=16.2, 4.9 Hz, 2H), 1.12 (d, J=6.0 Hz, 6H)

LCMS (Method 4): 2.28 min, 394.2 [M+H]⁺

### Synthesis of 3-cyano-N-(3,3-difluorocyclobutyl)-2-(isopropoxymethyl)pyrazolo[1,5-a]pyrimidine-7-carboxamide

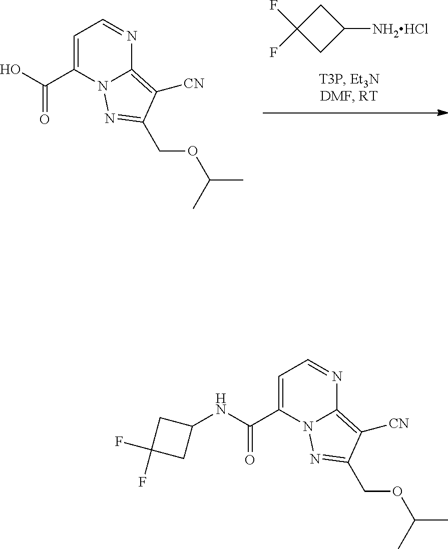

Et₃N (161 μL, 1.15 mmol) and 1-propanephosphonic anhydride (50 wt % in DMF; 690 μL, 1.15 mmol) were added to a solution of 3-cyano-2-(isopropoxymethyl)pyrazolo[1,5-a]pyrimidine-7-carboxylic acid (100 mg, 0.38 mmol) and 3,3-difluorocyclobutanamine hydrochloride (61 mg, 0.42 mmol) in DMF (1.3 mL) and the reaction was stirred at room temperature for 24 h. The reaction was quenched with water and diluted with DCM. The layers were separated, and the aqueous layer was extracted with DCM (×2). The combined organic layers were dried through a hydrophobic frit and concentrated in vacuo. The crude product was purified by column chromatography (eluting with 0 to 50% EtOAc in heptane) to provide the title compound (103 mg).

¹H NMR (400 MHZ, CDCl₃) δ 10.38 (d, J=6.2 Hz, 1H), 8.90 (d, J=4.3 Hz, 1H), 7.97 (d, J=4.3 Hz, 1H), 4.88 (s, 2H), 4.59-4.43 (m, 1H), 3.88 (hept, J=6.0 Hz, 1H), 3.26-3.09 (m, 2H), 2.81-2.64 (m, 2H), 1.33 (d, J=6.1 Hz, 6H)

LCMS (Method 1): 1.75 min, 350.2 [M+H]⁺

### Synthesis of compound 28, N7-(3,3-difluorocyclobutyl)-2-(isopropoxymethyl)pyrazolo[1,5-a]pyrimidine-3,7-dicarboxamide

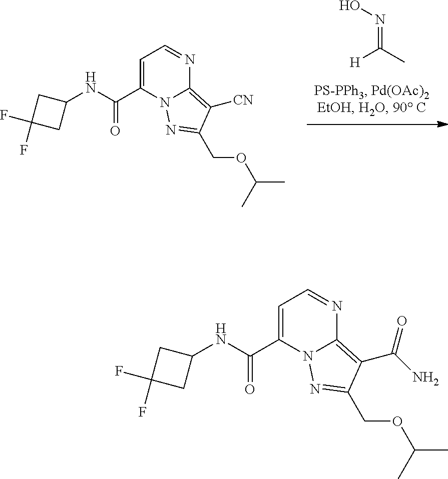

3-Cyano-N-(3,3-difluorocyclobutyl)-2-(isopropoxymethyl)pyrazolo[1,5-a]pyrimidine-7-carboxamide (102 mg, 0.29 mmol), acetaldehyde oxime (71 μL, 1.17 mmol), Pd(OAc)₂ (10 mg, 0.04 mmol), and PS-PPh₃ (1.4-2 mmol/g; 30 mg, 0.04-0.06 mmol) were suspended in EtOH (2.2 mL) and water (550 μL) and the reaction was heated at 90° C. for 2 h. The reaction mixture was cooled to room temperature and diluted with DCM. The layers were separated, and the aqueous layer was extracted with DCM (×2). The combined organic layers were dried through a hydrophobic frit and concentrated in vacuo. The crude product was purified by column chromatography (eluting with 0 to 5% MeOH in DCM) to provide the title compound (82 mg).

¹H NMR (400 MHz, DMSO-d₆) δ 10.32 (d, J=6.4 Hz, 1H), 8.92 (d, J=4.4 Hz, 1H), 7.68 (s, 1H), 7.66 (d, J=4.4 Hz, 1H), 7.58 (s, 1H), 5.04 (s, 2H), 4.44-4.31 (m, 1H), 3.82 (hept, J=6.1 Hz, 1H), 3.21-3.06 (m, 2H), 2.82-2.66 (m, 2H), 1.18 (d, J=6.1 Hz, 6H)

LCMS (Method 4): 2.11 min, 368.2 [M+H]⁺

### Synthesis of (5-amino-1H-pyrazol-3-yl)methanol

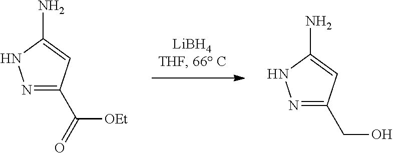 [SMILES: CCOC(=O)c1cc(N)[nH]n1 ; Nc1cc(CO)n[nH]1]

LiBH₄ (2 M in THF; 24.2 mL, 48.34 mmol) was added slowly to a solution of ethyl 5-amino-1H-pyrazole-3-carboxylate (5 g, 32.23 mmol) in THF (75 mL) and the reaction was heated at 66° C. overnight. The reaction mixture was cooled to room temperature, quenched with MeOH, and concentrated in vacuo. The crude product was purified by column chromatography (eluting with 0 to 20% MeOH in DCM) to provide the title compound (2.25 g).

¹H NMR (400 MHz, DMSO-d₆) δ 5.29 (s, 1H), 4.94 (s, 1H), 4.27 (s, 2H). Additional exchangeable protons not observed.

### Synthesis of ethyl 2-(hydroxymethyl)pyrazolo[1,5-a]pyrimidine-7-carboxylate

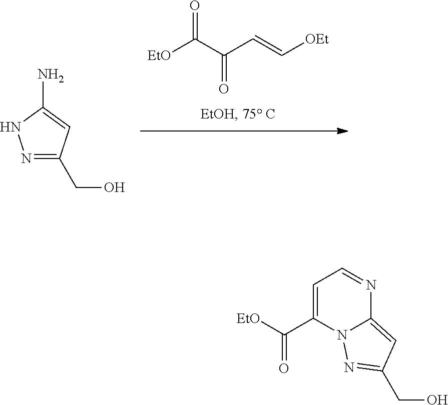 [SMILES: Nc1cc(CO)n[nH]1 ; CCO/C=C/C(=O)C(=O)OCC ; CCOC(=O)c1ccnc2cc(CO)nn12]

Ethyl (E)-4-ethoxy-2-oxo-but-3-enoate (4.11 g, 23.87 mmol) and (5-amino-1H-pyrazol-3-yl)methanol (2.25 g, 19.89 mmol) were dissolved in EtOH (75 mL) and the reaction was heated at 75° C. for 4 h. The reaction mixture was cooled to room temperature and concentrated in vacuo. The crude product was purified by column chromatography (eluting with 0 to 20% MeOH in DCM) to provide the title compound (3.51 g).

¹H NMR (400 MHz, DMSO-d₆) δ 8.64 (d, J=4.2 Hz, 1H), 7.38 (d, J=4.2 Hz, 1H), 6.82 (s, 1H), 5.44 (t, J=5.8 Hz, 1H), 4.68 (d, J=4.9 Hz, 2H), 4.48 (q, J=7.1 Hz, 2H), 1.37 (t, J=7.1 Hz, 3H)

LCMS (Method 1): 0.56 min, 222.2 [M+H]⁺

### Synthesis of ethyl 3-bromo-2-(hydroxymethyl)pyrazolo[1,5-a]pyrimidine-7-carboxylate

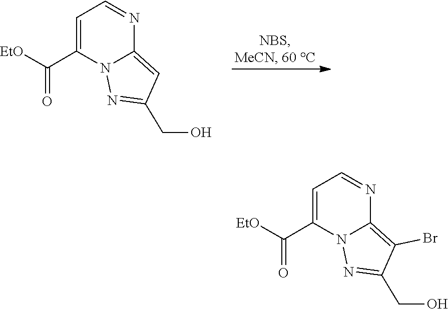 [SMILES: CCOC(=O)c1ccnc2cc(CO)nn12 ; CCOC(=O)c1ccnc2c(Br)c(CO)nn12]

NBS (454 mg, 2.55 mmol) was added to a solution of ethyl 2-(hydroxymethyl)pyrazolo[1,5-a]pyrimidine-7-carboxylate (470 mg, 2.13 mmol) in MeCN (11 mL) and the reaction was heated at 60° C. for 18 h. The reaction mixture was cooled to room temperature and concentrated in vacuo. The residue was dissolved in DCM and washed with sat. aq. NaHCO₃ (×3). The organic layer was dried through a hydrophobic frit and concentrated in vacuo to provide the title compound (699 mg). The crude product was taken forward to the next step without further purification.

¹H NMR (400 MHZ, CDCl₃) δ 8.64 (d, J=4.1 Hz, 1H), 7.35 (d, J=4.1 Hz, 1H), 4.99 (s, 2H), 4.56 (q, J=7.2 Hz, 2H), 1.47 (t, J=7.1 Hz, 3H). Exchangeable proton not observed.

### Synthesis of 3-bromo-2-(hydroxymethyl)pyrazolo[1,5-a]pyrimidine-7-carboxylic Acid

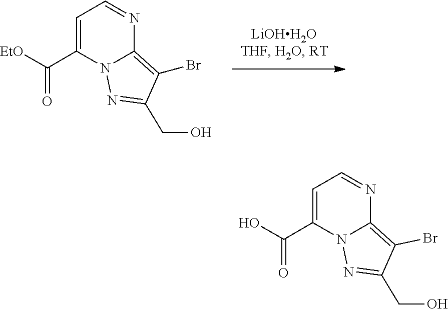 [SMILES: CCOC(=O)c1ccnc2c(Br)c(CO)nn12 ; O=C(O)c1ccnc2c(Br)c(CO)nn12]

LiOH·H₂O (147 mg, 3.49 mmol) was added to a solution of ethyl 3-bromo-2-(hydroxymethyl)pyrazolo[1,5-a]pyrimidine-7-carboxylate (699 mg, 2.33 mmol) in THF (5.8 mL) and water (5.8 mL) and the reaction was stirred at room temperature for 18 h. The reaction mixture was diluted with EtOAc and water and acidified to pH 2-3 with 1 M aq. HCl. The layers were separated, and the aqueous layer was extracted with EtOAc (×3). The combined organic layers were dried through a hydrophobic frit and concentrated in vacuo to provide the title compound (394 mg).

¹H NMR (400 MHz, DMSO-d₆) δ 8.71 (d, J=4.1 Hz, 1H), 7.44 (d, J=4.1 Hz, 1H), 4.66 (s, 2H). Exchangeable protons not observed.

LCMS (Method 2): 1.14 min, 272.0, 274.0 [M+H]₊

### Synthesis of 3-bromo-2-(hydroxymethyl)-N-indan-2-yl-pyrazolo[1,5-a]pyrimidine-7-carboxamide

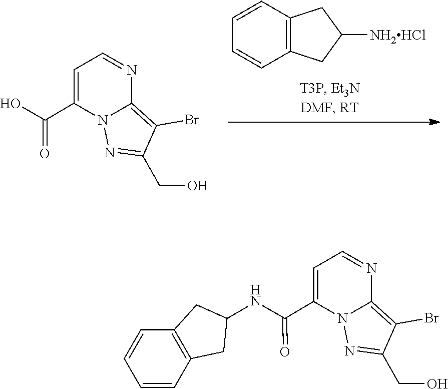

Indan-2-amine hydrochloride (369 mg, 2.17 mmol), 1-propanephosphonic anhydride (50 wt % in DMF; 1.69 mL, 2.9 mmol), and Et₃N (1.01 mL, 7.24 mmol) were added to a solution of 3-bromo-2-(hydroxymethyl)pyrazolo[1,5-a]pyrimidine-7-carboxylic acid (394 mg, 1.45 mmol) in DMF (10 mL) and the reaction was stirred at room temperature for 72 h. The reaction mixture was poured into water and diluted with EtOAc. The layers were separated, and the aqueous layer was extracted with EtOAc (×2). The combined organic layers were washed with sat. aq. LiCl (×2), dried through a hydrophobic frit, and concentrated in vacuo. The crude product was purified by column chromatography (eluting with 0 to 100% EtOAc in heptane) to provide the title compound (217 mg).

¹H NMR (400 MHz, DMSO-d₆) δ 10.10 (d, J=7.1 Hz, 1H), 8.75 (d, J=4.2 Hz, 1H), 7.56 (d, J=4.2 Hz, 1H), 7.27 (dd, J=5.4, 3.3 Hz, 2H), 7.22-7.14 (m, 2H), 5.49 (t, J=5.9 Hz, 1H), 4.85-4.72 (m, 1H), 4.65 (d, J=5.8 Hz, 2H), 3.45-3.28 (m, 2H), 3.01 (dd, J=16.0, 6.0 Hz, 2H)

LCMS (Method 2): 2.25 min, 387.0, 389.0 [M+H]⁺

### Synthesis of [3-bromo-7-(indan-2-ylcarbamoyl)pyrazolo[1,5-a]pyrimidin-2-yl]methyl methanesulfonate

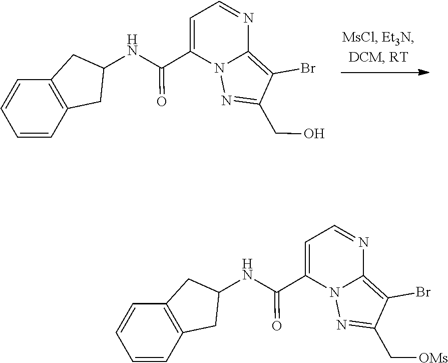 [SMILES: O=C(NC1Cc2ccccc2C1)c1ccnc2c(Br)c(CO)nn12 ; CS(=O)(=O)OCc1nn2c(C(=O)NC3Cc4ccccc4C3)ccnc2c1Br]

Et₃N (85 μL, 0.61 mmol) and methanesulfonyl chloride (35 μL, 0.45 mmol) were added to a solution of 3-bromo-2-(hydroxymethyl)-N-indan-2-yl-pyrazolo[1,5-a]pyrimidine-7-carboxamide (158 mg, 0.41 mmol) in DCM (3 mL) and the reaction was stirred at room temperature for 2 h. The reaction mixture was diluted with sat. aq. NaHCO₃ and DCM and the layers were separated. The organic layer was washed with sat. aq. NaHCO₃ (×2), dried through a hydrophobic frit, and concentrated in vacuo to provide the title compound (185 mg).

¹H NMR (400 MHZ, CDCl₃) δ 10.29 (d, J=7.0 Hz, 1H), 8.79 (d, J=4.2 Hz, 1H), 7.94 (d, J=4.2 Hz, 1H), 7.33-7.15 (m, 4H), 5.41 (s, 2H), 5.03-4.91 (m, 1H), 3.47 (dd, J=16.0, 7.1 Hz, 2H), 3.18-3.01 (m, 2H), 2.97 (s, 3H)

LCMS (Method 5): 1.12 min, 465.0, 467.0 [M+H]⁺

### Synthesis of 3-bromo-2-[(dimethylamino)methyl]-N-indan-2-yl-pyrazolo[1,5-a]pyrimidine-7-carboxamide

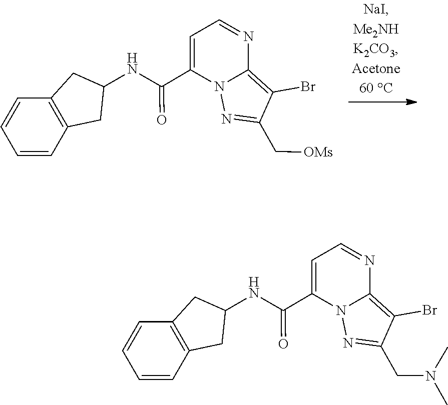 [SMILES: CS(=O)(=O)OCc1nn2c(C(=O)NC3Cc4ccccc4C3)ccnc2c1Br ; CN(C)Cc1nn2c(C(=O)NC3Cc4ccccc4C3)ccnc2c1Br]

Sodium Iodide (58 mg, 0.39 mmol) was added to a solution of [3-bromo-7-(indan-2-ylcarbamoyl)pyrazolo[1,5-a]pyrimidin-2-yl]methyl methanesulfonate (180 mg, 0.39 mmol) in acetone (2 mL) and the reaction was heated at 60° C. for 1 h. Me₂NH (2 M in THF; 290 μL, 0.58 mmol) and K₂CO₃ (160 mg, 1.16 mmol) were added and the reaction was heated at 60° C. for a further 1 h. The reaction mixture was cooled to room temperature and concentrated in vacuo. The residue was dissolved in DCM and washed with water. The aqueous layer was extracted with DCM, and the combined organic layers were dried over Na₂SO₄ and concentrated in vacuo to provide the title compound (151 mg).

¹H NMR (400 MHz, CDCl₃) δ 10.61 (d, J=7.1 Hz, 1H), 8.72 (d, J=4.3 Hz, 1H), 7.84 (d, J=4.3 Hz, 1H), 7.28 (dd, J=5.4, 3.4 Hz, 2H), 7.23-7.19 (m, 2H), 5.06-4.92 (m, 1H), 3.72 (s, 2H), 3.46 (dd, J=16.1, 6.9 Hz, 2H), 3.04 (dd, J=16.0, 4.6 Hz, 2H), 2.26 (s, 6H)

### Synthesis of Compound 29, 2-[(dimethylamino)methyl]-N7-indan-2-yl-pyrazolo[1,5-a]pyrimidine-3,7-dicarboxamide

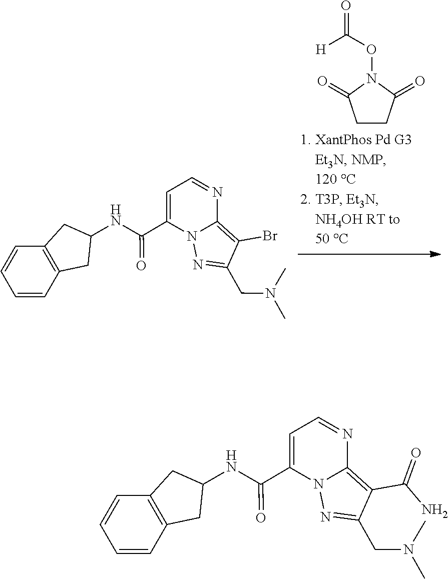 [SMILES: O=CON1C(=O)CCC1=O ; CN(C)Cc1nn2c(C(=O)NC3Cc4ccccc4C3)ccnc2c1Br ; CN(C)Cc1nn2c(C(=O)NC3Cc4ccccc4C3)ccnc2c1C(N)=O]

3-Bromo-2-[(dimethylamino)methyl]-N-indan-2-yl-pyrazolo[1,5-a]pyrimidine-7-carboxamide (150 mg, 0.36 mmol), XantPhos Pd G3 (34 mg, 0.04 mmol), Et₃N (150 μL, 1.09 mmol), and NMP (2 mL) were placed in a vial and the vial was sealed and purged with argon for 15 min. (2,5-Dioxopyrrolidin-1-yl) formate (259 mg, 1.81 mmol) was added and the vial was sealed immediately. The reaction was heated at 120° C. in a microwave reactor for 4 h, and then for a further 6 h, during which additional portions of XantPhos Pd G3 (69 mg, 0.08 mmol), Et₃N (300 μL, 2.18 mmol), and (2,5-dioxopyrrolidin-1-yl) formate (518 mg, 3.62 mmol) were added in total. The reaction mixture was cooled to room temperature and Et₃N (150 μL, 1.09 mmol), 1-propanephosphonic anhydride (50 wt % in EtOAc; 430 μL, 0.72 mmol) and NH₄OH (470 μL, 3.62 mmol) were added. The reaction was stirred at room temperature for 16 h, and then heated at 50° C. for further 4 days, during which additional portions of Et₃N (300 μL, 2.18 mmol), 1-propanephosphonic anhydride (50 wt % in EtOAc; 860 μL, 1.44 mmol) and NH₄OH (940 μL, 7.24 mmol) were added in total. The reaction mixture was cooled to room temperature and diluted with water and EtOAc. The layers were separated, and the aqueous layer was extracted with EtOAc (×2). The combined organic layers were dried over Na₂SO₄ and concentrated in vacuo. The crude product was purified by preparative HPLC (acidic method), SCX chromatography (eluting with 2 M NH₃ in MeOH), and column chromatography (eluting with 0 to 10% MeOH in DCM) to provide the title compound (8.5 mg).

¹H NMR (400 MHz, DMSO-d₆) δ 10.30 (d, J=7.1 Hz, 1H), 8.89 (d, J=4.4 Hz, 1H), 7.90 (s, 1H), 7.70 (d, J=4.4 Hz, 1H), 7.48 (s, 1H), 7.29 (dd, J=5.4, 3.3 Hz, 2H), 7.23-7.14 (m, 2H), 4.87-4.75 (m, 1H), 3.96 (s, 2H), 3.38 (dd, J=16.2, 7.0 Hz, 2H), 2.96 (dd, J=16.2, 4.1 Hz, 2H), 2.16 (s, 6H)

LCMS (Method 2): 1.27 min, 379.2 [M+H]⁺

### Synthesis of 3-bromo-2-formyl-N-indan-2-yl-pyrazolo[1,5-a]pyrimidine-7-carboxamide

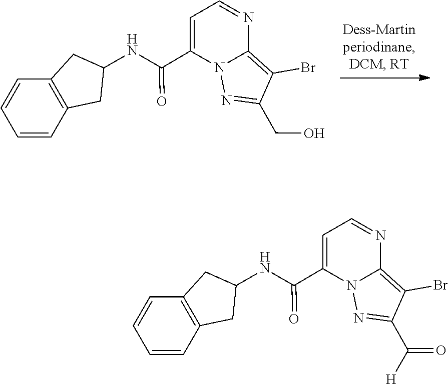 [SMILES: O=C(NC1Cc2ccccc2C1)c1ccnc2c(Br)c(CO)nn12 ; O=Cc1nn2c(C(=O)NC3Cc4ccccc4C3)ccnc2c1Br]

Dess-Martin periodinane (263 mg, 0.62 mmol) was added in one portion to a solution of 3-bromo-2-(hydroxymethyl)-N-indan-2-yl-pyrazolo[1,5-a]pyrimidine-7-carboxamide (200 mg, 0.52 mmol) in DCM (12 mL) and the reaction was stirred at room temperature for 4 h. An additional portion of Dess-Martin periodinane (263 mg, 0.62 mmol) was added and the reaction was stirred at room temperature for a further 2 h. The reaction mixture was diluted with DCM and washed with sat. aq. NaHCO₃. The aqueous layer was extracted with DCM (×2), and the combined organic layers were dried through a hydrophobic frit and concentrated in vacuo. The crude product was purified by column chromatography (eluting with 0 to 50% EtOAc in heptane) to provide the title compound (141 mg).

¹H NMR (400 MHz, DMSO-d₆) δ10.12 (s, 1H), 9.74 (d, J=7.1 Hz, 1H), 8.88 (d, J=4.2 Hz, 1H), 7.68 (d, J=4.3 Hz, 1H), 7.32-7.23 (m, 2H), 7.22-7.14 (m, 2H), 4.86-4.73 (m, 1H), 3.37 (dd, J=16.1, 7.6 Hz, 2H), 3.01 (dd, J=16.1, 5.6 Hz, 2H) LCMS (Method 1): 1.32 min, 385.0, 387.0 [M+H]⁺

### Synthesis of 3-bromo-N-indan-2-yl-2-(methylaminomethyl)pyrazolo[1,5-a]pyrimidine-7-carboxamide

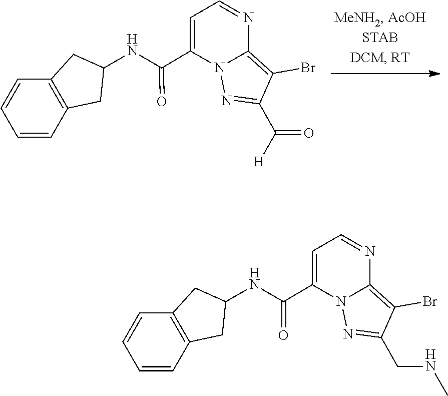 [SMILES: O=Cc1nn2c(C(=O)NC3Cc4ccccc4C3)ccnc2c1Br ; CNCc1nn2c(C(=O)NC3Cc4ccccc4C3)ccnc2c1Br]

MeNH₂ (2 M in THF; 930 μL, 1.86 mmol) and acetic acid (36 μL, 0.62 mmol) were added sequentially to a solution of 3-bromo-2-formyl-N-indan-2-yl-pyrazolo[1,5-a]pyrimidine-7-carboxamide (143 mg, 0.37 mmol) in DCM (7 mL) and the reaction was stirred at room temperature for 1 h. Sodium triacetoxyborohydride (118 mg, 0.56 mmol) was added in one portion, and the reaction was stirred at room temperature for 2 h. Additional sodium triacetoxyborohydride (50 mg, 0.24 mmol) was added and the reaction was stirred at room temperature for a further 2 h. The reaction mixture was diluted with DCM and washed with sat. aq. NaHCO₃. The aqueous layer was extracted with DCM (×2), and the combined organic layers were dried through a hydrophobic frit and concentrated in vacuo. The crude product was purified by column chromatography (eluting with 0 to 5% (2 M NH₃ in MeOH) in DCM) to provide the title compound (125 mg).

¹H NMR (400 MHz, CDCl₃) δ10.61 (d, J=5.6 Hz, 1H), 8.71 (d, J=4.3 Hz, 1H), 7.82 (d, J=4.3 Hz, 1H), 7.33-7.28 (m, 2H), 7.25-7.21 (m, 2H), 5.08-4.96 (m, 1H), 3.91 (s, 2H), 3.45 (dd, J=16.0, 6.8 Hz, 2H), 3.04 (dd, J=16.0, 4.2 Hz, 2H), 2.37 (s, 3H). Exchangeable proton not observed.

LCMS (Method 1): 0.66 min, 0.74 min, 400.0, 402.0 [M+H]⁺

### Synthesis of 2-[[formyl(methyl)amino]methyl]-N7-indan-2-yl-pyrazolo[1,5-a]pyrimidine-3,7-dicarboxamide

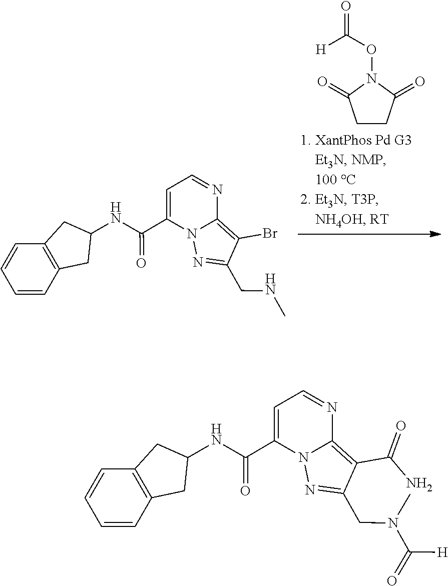 [SMILES: O=CON1C(=O)CCC1=O ; CNCc1nn2c(C(=O)NC3Cc4ccccc4C3)ccnc2c1Br ; CN(C=O)Cc1nn2c(C(=O)NC3Cc4ccccc4C3)ccnc2c1C(N)=O]

3-Bromo-N-indan-2-yl-2-(methylaminomethyl)pyrazolo[1,5-a]pyrimidine-7-carboxamide (125 mg, 0.31 mmol), NMP (3 mL), and Et₃N (131 μL, 0.94 mmol) were placed in a vial and the vial was sealed and purged with argon for 5 min. XantPhos Pd G3 (30 mg, 0.03 mmol) was added and the vial was purged with argon for a further 5 min. (2,5-Dioxopyrrolidin-1-yl) formate (223 mg, 1.56 mmol) was added and the vial was sealed immediately. The reaction was heated at 100° C. in a microwave reactor for 2 h then cooled to room temperature. Et₃N (131 μL, 0.94 mmol), 1-propanephosphonic anhydride (50 wt % in DMF; 372 μL, 0.62 mmol), and NH₄OH (405 μL, 3.12 mmol) were added and the reaction was stirred at room temperature for 5 h. The reaction mixture was diluted with EtOAc, filtered through a short pad of Dicalite®, and concentrated in vacuo to a small volume (~3 mL). The solution of crude product was taken forward to the next step without further purification and assuming 100% conversion to the title compound.

LCMS (Method 3): 1.05 min, 1.08 min, 393.2 [M+H]⁺

### Synthesis of Compound 30, N7-indan-2-yl-2-(methylaminomethyl)pyrazolo[1,5-a]pyrimidine-3,7-dicarboxamide

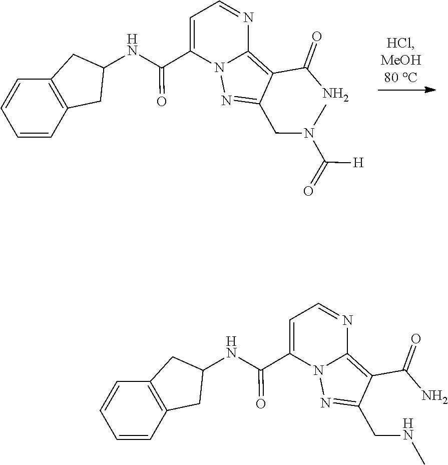 [SMILES: CN(C=O)Cc1nn2c(C(=O)NC3Cc4ccccc4C3)ccnc2c1C(N)=O ; CNCc1nn2c(C(=O)NC3Cc4ccccc4C3)ccnc2c1C(N)=O]

1 M aq. HCl (10 mL, 10.00 mmol) was added to the solution of 2-[[formyl(methyl)amino]methyl]-N7-indan-2-yl-pyrazolo[1,5-a]pyrimidine-3,7-dicarboxamide generated in the previous step (~3 mL) in MeOH (9.4 mL) and the reaction was heated at 80° C. for 8 h. The reaction mixture was cooled to room temperature and the crude product was purified by SCX chromatography (eluting with 2 M NH₃ in MeOH) and column chromatography (eluting with 0 to 5% (2 M NH₃ in MeOH) in DCM) to provide the title compound (57 mg).

¹H NMR (400 MHz, DMSO-d₆) δ 10.24 (d, J=7.2 Hz, 1H), 8.89 (d, J=4.4 Hz, 1H), 7.82 (s, 1H), 7.68 (dd, J=4.4, 0.8 Hz, 1H), 7.59 (s, 1H), 7.34-7.25 (m, 2H), 7.24-7.15 (m, 2H), 4.87-4.75 (m, 1H), 3.98 (s, 2H), 3.36 (dd, J=16.1, 7.1 Hz, 2H), 3.00 (dd, J=16.0, 4.8 Hz, 2H), 2.20 (s, 3H). Additional exchangeable proton not observed.

LCMS (Method 2): 1.48 min, 365.2 [M+H]⁺

### Synthesis of 2-pyrazolo[1,5-a]pyridin-3-yloxazole

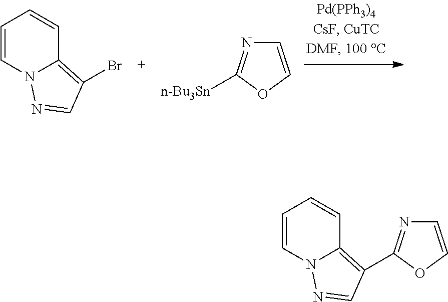

3-Bromopyrazolo[1,5-a]pyridine (500 mg, 2.54 mmol) was dissolved in DMF (5 mL) and the solution was degassed by sparging with a stream of argon. Tributyl(oxazol-2-yl) stannane (1 mL, 3.30 mmol), Pd(PPh₃)₄ (150 mg, 0.13 mmol), thiophene-2-carbonyloxycopper (629 mg, 3.30 mmol), and CsF (501 mg, 3.30 mmol) were added and the reaction was heated at 100° C. for 3 h. The reaction mixture was cooled to room temperature, diluted with EtOAc, and filtered through a short pad of Dicalite®. The filtrate was washed with 5% aq. LiCl (×2) and sat. aq. NaHCO₃, and the organic layer was dried over Na₂SO₄. A mixture of KF (10 wt %) in silica gel was added, and the mixture was stirred at room temperature for 1 h then concentrated in vacuo. The crude product was purified by column chromatography (eluting with 0 to 100% EtOAc in heptane) to provide the title compound (243 mg).

¹H NMR (400 MHz, CDCl₃) δ 8.57-8.50 (m, 1H), 8.46 (s, 1H), 8.30-8.23 (m, 1H), 7.67 (d, J=0.9 Hz, 1H), 7.38-7.33 (m, 1H), 7.22 (d, J=0.8 Hz, 1H), 6.96-6.88 (m, 1H)

LCMS (Method 1): 1.48 min, 186.2 [M+H]⁺

### Synthesis of ethyl 2-(7-bromopyrazolo[1,5-a]pyridin-3-yl) oxazole

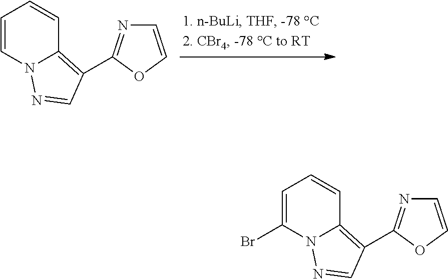 [SMILES: c1ccn2ncc(-c3ncco3)c2c1 ; Brc1cccc2c(-c3ncco3)cnn12]

n-BuLi (2.5 M in hexanes; 0.29 mL, 0.71 mmol) was added to a solution of 2-pyrazolo[1,5-a]pyridin-3-yloxazole (110 mg, 0.59 mmol) in THF (3 mL) at −78° C. and the mixture was stirred for 15 min. Carbon tetrabromide (256 mg, 0.77 mmol) was added in one portion, and the reaction was stirred at −78° C. for 45 min. The reaction mixture was warmed to room temperature, stirred for 1.5 h, then quenched with water and diluted with EtOAc. The layers were separated, and the aqueous layer was extracted with EtOAc. The combined organic layers were washed with brine, dried over Na₂SO₄, and concentrated in vacuo. The crude product was purified by column chromatography (eluting with 0 to 100% EtOAc in heptane) to provide the title compound (35 mg).

¹H NMR (400 MHz, CDCl₃) δ 8.60 (s, 1H), 8.39-8.30 (m, 1H), 7.72 (d, J=0.9 Hz, 1H), 7.29-7.28 (m, 1H), 7.28-7.26 (m, 2H)

LCMS (Method 1): 1.65 min, 264.2 [M+H]⁺

### Synthesis of Compound 31, N-indan-2-yl-3-oxazol-2-yl-pyrazolo[1,5-a]pyridine-7-carboxamide

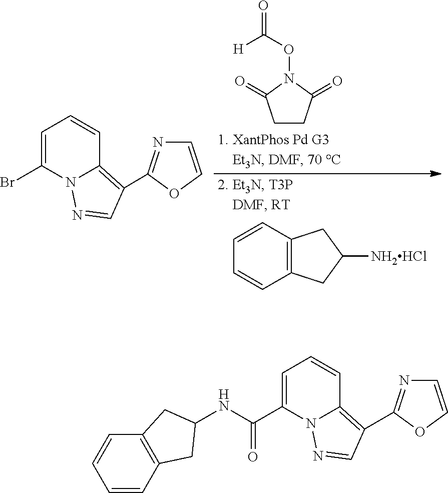

2-(7-Bromopyrazolo[1,5-a]pyridin-3-yl) oxazole (35 mg, 0.13 mmol) was dissolved in DMF (1 mL) and the solution was degassed by sparging with a stream of argon. (2,5-Dioxopyrrolidin-1-yl) formate (38 mg, 0.27 mmol), XantPhos Pd G3 (13 mg, 0.01 mmol), and Et₃N (40 μL, 0.27 mmol) were added, and the vial was sealed immediately. The reaction was heated at 70° C. for 2 h then cooled to room temperature. A solution of indan-2-amine hydrochloride (45 mg, 0.27 mmol) and Et₃N (40 μL, 0.27 mmol) in DMF (500 μL) was added dropwise and the reaction was stirred at room temperature for 1 h. 1-Propanephosphonic anhydride (50 wt % in DMF; 160 μL, 0.27 mmol) was added, and the reaction was stirred for a further 60 h. The reaction mixture was diluted with EtOAc and washed with 5% aq. LiCl (×2) and brine. The organic layer was dried over Na₂SO₄, filtered through a plug of silica gel, and concentrated in vacuo. The crude product was purified by preparative HPLC (acidic method) to provide the title compound (21 mg).

¹H NMR (400 MHz, CDCl₃) δ10.85 (d, J=7.1 Hz, 1H), 8.52 (dd, J=8.9, 1.5 Hz, 1H), 8.47 (s, 1H), 8.16 (dd, J=7.2, 1.5 Hz, 1H), 7.73 (d, J=0.9 Hz, 1H), 7.55 (dd, J=8.8, 7.2 Hz, 1H), 7.31 (dd, J=5.4, 3.4 Hz, 2H), 7.27 (d, J=0.8 Hz, 1H), 7.26-7.21 (m, 2H), 5.09-4.96 (m, 1H), 3.54 (dd, J=16.0, 7.6 Hz, 2H), 3.13 (dd, J=16.0, 6.1 Hz, 2H)

LCMS (Method 2): 2.71 min, 345.2 [M+H]⁺

### Synthesis of ethyl 3-cyano-2-methyl-pyrazolo[1,5-a]pyrimidine-7-carboxylate

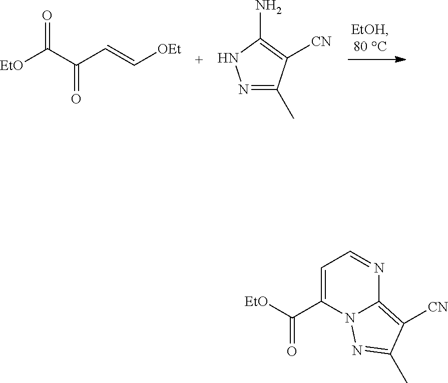

Ethyl (E)-4-ethoxy-2-oxo-but-3-enoate (10.86 g, 63.05 mmol) was added to a solution of 5-amino-3-methyl-1H-pyrazole-4-carbonitrile (7 g, 57.32 mmol) in EtOH (200 mL) and the reaction was heated at 80° C. for 1 h. The reaction mixture was cooled to room temperature and concentrated in vacuo to 50% solvent volume. The solution was cooled to 0° C. and left to stand for 1 h. The mixture was warmed to room temperature overnight, and the resulting precipitate was collected by filtration and dried to provide the title compound (6.05 g).

¹H NMR (400 MHz, DMSO-d₆) δ 8.90 (d, J=4.4 Hz, 1H), 7.67 (d, J=4.4 Hz, 1H), 4.47 (q, J=7.1 Hz, 2H), 2.56 (s, 3H), 1.36 (t, J=7.1 Hz, 3H)

LCMS (Method 2): 1.91 min, 231.2 [M+H]⁺

### Synthesis of 3-cyano-2-methyl-pyrazolo[1,5-a]pyrimidine-7-carboxylic Acid

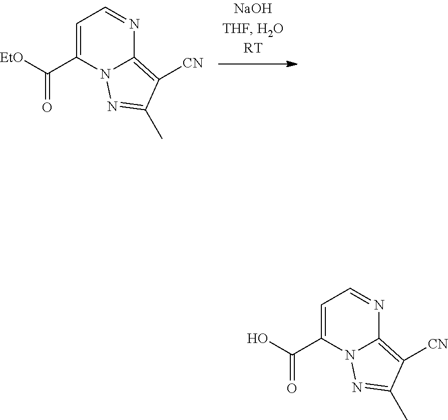 [SMILES: CCOC(=O)c1ccnc2c(C#N)c(C)nn12 ; Cc1nn2c(C(=O)O)ccnc2c1C#N]

2 M aq. NaOH (60.8 mL, 121.62 mmol) was added to a solution of ethyl 3-cyano-2-methyl-pyrazolo[1,5-a]pyrimidine-7-carboxylate (5.6 g, 24.32 mmol) in THF (45 mL) and MeOH (5 mL) and the reaction was stirred at room temperature for 3 h. The reaction mixture was acidified with 1 M aq. HCl and cooled to 5° C. The resulting precipitate was collected by filtration and suspended in water. The aqueous suspension was extracted with EtOAc (×5), and the combined organic layers were concentrated in vacuo to provide the title compound (4.51 g).

¹H NMR (400 MHz, DMSO-d₆) δ8.88 (d, J=4.3 Hz, 1H), 7.60 (d, J=4.4 Hz, 1H), 2.57 (s, 3H). Exchangeable proton not observed.

LCMS (Method 1) 0.72 min, 159.2 [M-CO₂H+H]⁺

### Synthesis of 3-cyano-N-(3,3-difluorocyclobutyl)-2-methyl-pyrazolo[1,5-a]pyrimidine-7-carboxamide

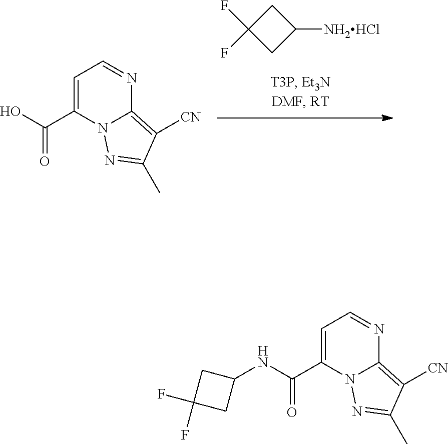

1-Propanephosphonic anhydride (50 wt % in DMF; 6.8 mL, 11.65 mmol) was added dropwise to a solution of 3-cyano-2-methyl-pyrazolo[1,5-a]pyrimidine-7-carboxylic acid (833 mg, 4.12 mmol), 3,3-difluorocyclobutanamine hydrochloride (888 mg, 6.19 mmol) and Et₃N (3.1 mL, 22.24 mmol) in DMF (5 mL) and the reaction was stirred at room temperature for 16 h. The reaction mixture was acidified with 1 M aq. HCl and diluted with water. The resulting precipitate was collected by filtration, washed with water, and dried to provide the title compound (756 mg).

¹H NMR (400 MHz, DMSO-d₆) δ 9.90 (d, J=6.5 Hz, 1H), 8.93 (d, J=4.4 Hz, 1H), 7.65 (d, J=4.4 Hz, 1H), 4.42-4.26 (m, 1H), 3.15-2.99 (m, 2H), 2.87-2.74 (m, 2H), 2.61 (s, 3H)

LCMS (Method 3): 0.98 min, 292.2 [M+H]⁺

### Synthesis of 6-bromo-3-cyano-N-(3,3-difluorocyclobutyl)-2-methyl-pyrazolo[1,5-a]pyrimidine-7-carboxamide

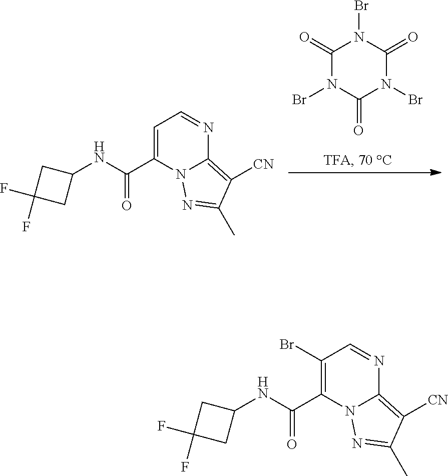 [SMILES: Cc1nn2c(C(=O)NC3CC(F)(F)C3)ccnc2c1C#N ; O=c1n(Br)c(=O)n(Br)c(=O)n1Br ; Cc1nn2c(C(=O)NC3CC(F)(F)C3)c(Br)cnc2c1C#N]

1,3,5-Tribromo-1,3,5-triazinane-2,4,6-trione (754 mg, 206 mmol) was added to a solution of 3-cyano-N-(3,3-difluorocyclobutyl)-2-methyl-pyrazolo[1,5-a]pyrimidine-7-carboxamide (400 mg, 1.37 mmol) in TFA (6.8 mL, 88.86 mmol) and the reaction was heated at 70° C. for 4 h.

The reaction mixture was cooled to room temperature and diluted with sat. aq. NaHCO₃ and EtOAc. The layers were separated, and the aqueous layer was extracted with EtOAc (×2). The combined organic layers were washed with sat. aq. Na₂SO₃ (×2), sat. aq. NaHCO₃(×2), and brine, dried over Na₂SO₄, and concentrated in vacuo. The crude product was purified by column chromatography (eluting with 0 to 60% EtOAc in heptane) to provide the title compound (306 mg).

¹H NMR (400 MHz, CDCl₃) δ8.71 (s, 1H), 7.79 (d, J=6.6 Hz, 1H), 4.55-4.40 (m, 1H), 3.19-3.04 (m, 2H), 2.72-2.58 (m, 2H), 2.55 (s, 3H)

LCMS (Method 1): 1.62 min, 370.0, 372.0 [M+H]⁺

### Synthesis of 3-cyano-N-(3,3-difluorocyclobutyl)-2-methyl-6-(1-tetrahydropyran-2-ylpyrazol-4-yl)pyrazolo[1,5-a]pyrimidine-7-carboxamide

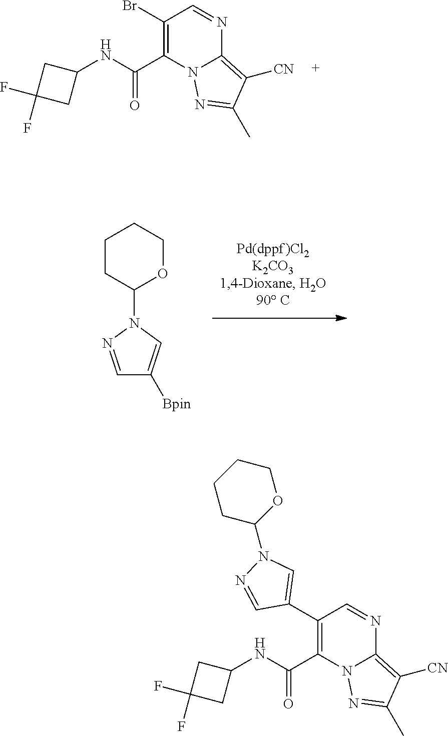

1-Tetrahydropyran-2-yl-4-(4,4,5,5-tetramethyl-1,3,2-dioxaborolan-2-yl) pyrazole (390 mg, 1.40 mmol), K₂CO₃ (280 mg, 2.03 mmol), and Pd(dppf)Cl₂ (50 mg, 0.07 mmol) were added to a solution of 6-bromo-3-cyano-N-(3,3-difluorocyclobutyl)-2-methyl-pyrazolo[1,5-a]pyrimidine-7-carboxamide (250 mg, 0.68 mmol) in 1,4-dioxane (2.3 mL) and water (233 μL) and the reaction was heated at 90° C. for 20 h. The reaction mixture was cooled to room temperature and diluted with EtOAc and water. The layers were separated, and the aqueous layer was extracted with EtOAc (×2). The combined organic layers were washed with brine, dried over Na₂SO₄, and concentrated in vacuo. The crude product was purified by column chromatography (eluting with 0 to 100% EtOAc in heptane) to provide the title compound (133 mg).

¹H NMR (400 MHz, CDCl₃) δ 8.77 (s, 1H), 7.95 (d, J=0.8 Hz, 1H), 7.77 (d, J=0.8 Hz, 1H), 7.10 (d, J=6.8 Hz, 1H), 5.42 (dd, J=9.1, 3.1 Hz, 1H), 4.56-4.42 (m, 1H), 4.13-4.06 (m, 1H), 3.78-3.68 (m, 1H), 3.18-3.03 (m, 2H), 2.66-2.49 (m, 5H), 2.19-2.00 (m, 3H), 1.77-1.62 (m, 3H)

### Synthesis of Compound 32, N7-(3,3-difluorocyclobutyl)-2-methyl-6-(1H-pyrazol-4-yl)pyrazolo[1,5-a]pyrimidine-3,7-dicarboxamide

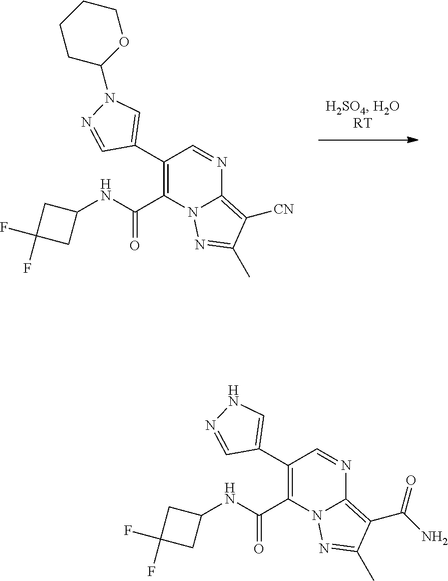 [SMILES: Cc1nn2c(C(=O)NC3CC(F)(F)C3)c(-c3cnn(C4CCCCO4)c3)cnc2c1C#N ; Cc1nn2c(C(=O)NC3CC(F)(F)C3)c(-c3cn[nH]c3)cnc2c1C(N)=O]

3-Cyano-N-(3,3-difluorocyclobutyl)-2-methyl-6-(1-tetrahydropyran-2-ylpyrazol-4-yl)pyrazolo[1,5-a]pyrimidine-7-carboxamide (133 mg, 0.30 mmol) was dissolved in H₂SO₄ (550 μL, 10.26 mmol) and water (61 μL, 3.39 mmol) and the reaction was stirred at room temperature for 4 h. The reaction mixture was neutralised with sat. aq. NaHCO₃ and extracted with EtOAc (×3). The combined organic layers were washed with brine, dried over Na₂SO₄, and concentrated in vacuo. The crude product was purified by preparative HPLC (basic and acidic methods) to provide the title compound (33 mg).

¹H NMR (400 MHz, DMSO-d₆) δ13.28 (s, 1H), 9.67 (d, J=6.2 Hz, 1H), 9.07 (s, 1H), 8.16 (s, 1H), 7.84 (s, 1H), 7.56 (s, 1H), 7.47 (s, 1H), 4.37-4.25 (m, 1H), 3.16-3.01 (m, 2H), 2.65-2.52 (m, 5H)

LCMS (Method 2): 1.31 min, 376.0 [M+H]⁺

### Synthesis of ethyl 2-bromo-3-cyano-pyrazolo[1,5-a]pyrimidine-7-carboxylate

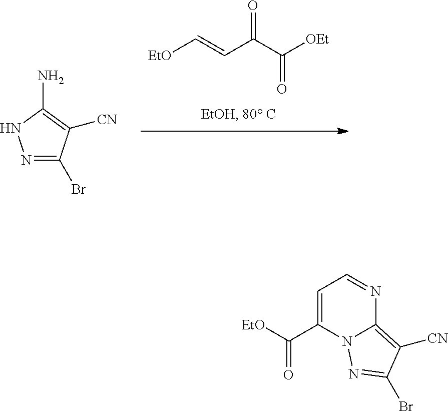 [SMILES: N#Cc1c(Br)n[nH]c1N ; CCO/C=C/C(=O)C(=O)OCC ; CCOC(=O)c1ccnc2c(C#N)c(Br)nn12]

5-Amino-3-bromo-1H-pyrazole-4-carbonitrile (5 g, 26.74 mmol) was added to a solution of ethyl (E)-4-ethoxy-2-oxo-but-3-enoate (5.06 g, 29.41 mmol) in EtOH (75 mL) and the reaction was heated at 80° C. for 4 h. An additional portion of ethyl (E)-4-ethoxy-2-oxo-but-3-enoate (3 g, 17.40 mmol) was added and the reaction was heated at 80° C. overnight. The reaction mixture was cooled to room temperature and concentrated in vacuo. Cold EtOH (10 mL) was added to the residue, and the resulting suspension was cooled to 0° C. and allowed to stand for 1 h. The precipitate was collected by filtration, washed with EtOH, and dried to provide the title compound (3.2 g).

¹H NMR (400 MHz, DMSO-d₆) δ 9.02 (d, J=4.4 Hz, 1H), 7.81 (d, J=4.4 Hz, 1H), 4.50 (q, J=7.1 Hz, 2H), 1.38 (t, J=7.1 Hz, 3H)

LCMS (Method 1): 0.98 min, 295.0, 297.0 [M+H]⁺

### Synthesis of 2-bromo-3-cyano-pyrazolo[1,5-a]pyrimidine-7-carboxylic Acid

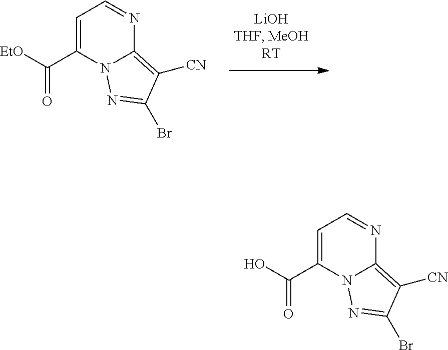 [SMILES: CCOC(=O)c1ccnc2c(C#N)c(Br)nn12 ; N#Cc1c(Br)nn2c(C(=O)O)ccnc12]

2 M aq. LiOH (930 μL, 1.86 mmol) was added dropwise to a solution of ethyl 2-bromo-3-cyano-pyrazolo[1,5-a]pyrimidine-7-carboxylate (500 mg, 1.69 mmol) in THF (2 mL) and EtOH (2 mL) and the reaction was stirred at room temperature for 3 h. The reaction mixture was concentrated in vacuo and the residue was diluted with water and acidified to pH 1-2 with 5 M aq. HCl. The aqueous suspension was extracted with a 1:19 mixture of MeOH:EtOAc (×3), and the combined organic layers were washed with brine, dried through a hydrophobic frit, and concentrated in vacuo to provide the title compound (435 mg).

¹H NMR (400 MHz, DMSO-d₆) δ 8.97 (d, J=4.4 Hz, 1H), 7.70 (d, J=4.4 Hz, 1H). Exchangeable proton not observed.

LCMS (Method 1): 0.80 min, 0.96 min (product does not ionise)

### Synthesis of 2-bromo-3-cyano-N-indan-2-yl-pyrazolo[1,5-a]pyrimidine-7-carboxamide

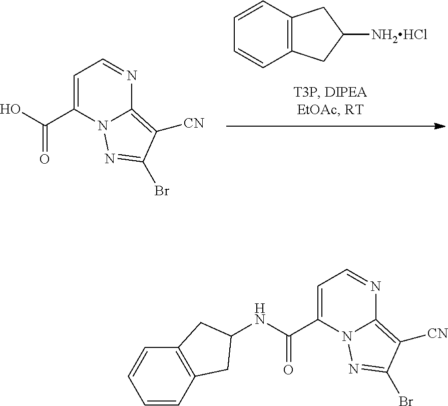

1-Propanephosphonic anhydride (50 wt % in EtOAc; 1.78 mL, 3.02 mmol) was added to a suspension of 2-bromo-3-cyano-pyrazolo[1,5-a]pyrimidine-7-carboxylic acid (403 mg, 1.51 mmol), indan-2-amine hydrochloride (307 mg, 1.81 mmol), and DIPEA (790 μL, 4.53 mmol) in EtOAc (7 mL) and the reaction was stirred at room temperature for 18 h. The reaction mixture was diluted with water and a 1:19 mixture of MeOH:EtOAc. The layers were separated, and the organic layer was washed with sat. aq. NH₄Cl and sat. aq. NaHCO₃, dried over Na₂CO₃, and concentrated in vacuo. The crude product was purified by column chromatography (eluting with 0 to 60% EtOAc in heptane) to provide the title compound (488 mg).

¹H NMR (400 MHz, CDCl₃) δ 9.86 (d, J=7.2 Hz, 1H), 8.91 (d, J=4.4 Hz, 1H), 8.03 (d, J=4.4 Hz, 1H), 7.33-7.18 (m, 4H), 5.04-4.91 (m, 1H), 3.54-3.45 (m, 2H), 3.05 (dd, J=15.9, 5.7 Hz, 2H)

LCMS (Method 1): 1.83 min, 382.0, 384.0 [M+H]⁺

### Synthesis of 3-cyano-N-indan-2-yl-2-oxazol-2-yl-pyrazolo[1,5-a]pyrimidine-7-carboxamide

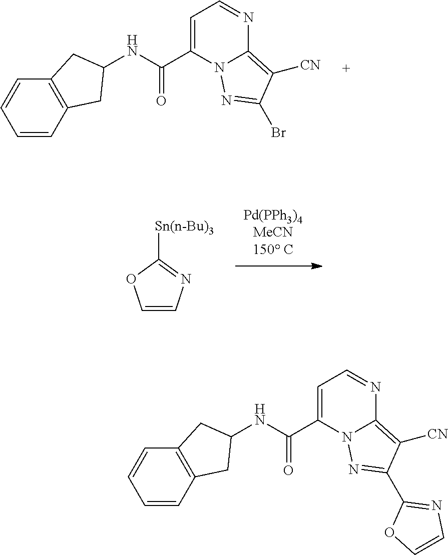

Pd(PPh₃)₄ (29 mg, 0.04 mmol), tributyl(oxazol-2-yl) stannane (170 μL, 0.55 mmol), and 2-bromo-3-cyano-N-indan-2-yl-pyrazolo[1,5-a]pyrimidine-7-carboxamide (150 mg, 0.39 mmol) were dissolved in MeCN (5 mL) and the solution was degassed by sparging with a stream of argon for 5 min. The reaction was heated at 150° C. in a microwave reactor for 2 h then cooled to room temperature. The reaction mixture was filtered through a short pad of Dicalite®, washed with EtOAc, and concentrated in vacuo. The crude product was purified by column chromatography (eluting with 0 to 100% EtOAc in heptane) to provide the title compound (10 mg).

¹H NMR (400 MHz, DMSO-d₆) δ 9.70 (d, J=7.2 Hz, 1H), 9.04 (d, J=4.4 Hz, 1H), 8.49 (d, J=0.8 Hz, 1H), 7.77 (d, J=4.4 Hz, 1H), 7.66 (d, J=0.8 Hz, 1H), 7.30 (t, J=4.4 Hz, 2H), 7.23-7.16 (m, 2H), 4.86-4.79 (m, 1H), 3.43 (d, J=2.3 Hz, 2H), 2.98 (dd, J=16.1, 5.0 Hz, 2H) LCMS (Method 1): 1.74 min, 371.0 [M+H]⁺

### Synthesis of Compound 33, N7-indan-2-yl-2-oxazol-2-yl-pyrazolo[1,5-a]pyrimidine-3,7-dicarboxamide

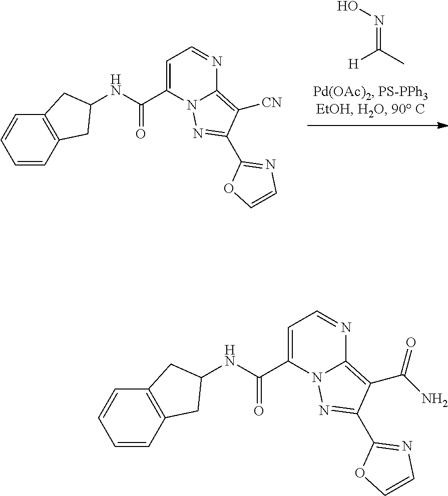

3-Cyano-N-indan-2-yl-2-oxazol-2-yl-pyrazolo[1,5-a]pyrimidine-7-carboxamide (17 mg, 0.05 mmol), acetaldehyde oxime (11 μL, 0.18 mmol), and PS-PPh₃ (1.4-2 mmol/g; 130 mg, 0.18-0.26 mmol) were dissolved in EtOH (4 mL) and water (1 mL), and the solution was degassed by sparging with a stream of argon for 5 min. Pd(OAc)₂ (1.6 mg, 0.01 mmol) was added and the reaction was heated at 90° C. for 3 days. The reaction mixture was cooled to room temperature, filtered through a short pad of Dicalite®, and washed with EtOAc, DCM, and MeOH. The filtrate was concentrated in vacuo, and the crude product was purified by column chromatography (eluting with 0 to 10% MeOH in DCM) and preparative HPLC (basic method) to provide the title compound (3.6 mg).

¹H NMR (400 MHz, DMSO-d₆) δ 9.88 (d, J=6.7 Hz, 1H), 8.95 (d, J=4.3 Hz, 1H), 8.36 (d, J=0.8 Hz, 1H), 8.01 (s, 1H), 7.66 (d, J=4.3 Hz, 1H), 7.65 (s, 1H), 7.52 (d, J=0.8 Hz, 1H), 7.28 (dd, J=5.4, 3.3 Hz, 2H), 7.18 (dd, J=5.5, 3.2 Hz, 2H), 4.83-4.74 (m, 1H), 3.45 (s, 2H), 2.97 (dd, J=16.1, 5.3 Hz, 2H)

LCMS (Method 2): 2.07 min, 389.2 [M+H]⁺

### Synthesis of 2-bromo-3-cyano-N-(3,3-difluorocyclobutyl)pyrazolo[1,5-a]pyrimidine-7-carboxamide

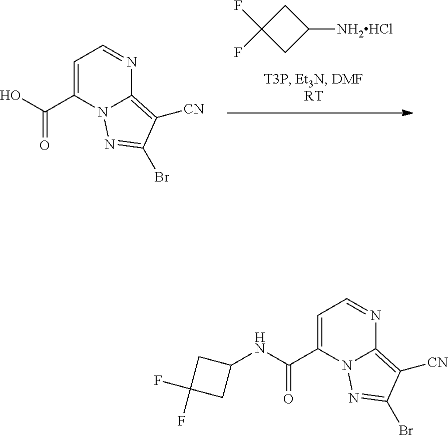

1-Propanephosphonic anhydride (50 wt % in DMF; 2.9 mL, 4.89 mmol) and Et₃N (681 μL, 4.89 mmol) were added to a solution of 2-bromo-3-cyano-pyrazolo[1,5-a]pyrimidine-7-carboxylic acid (435 mg, 1.63 mmol) and 3,3-difluorocyclobutanamine hydrochloride (351 mg, 2.44 mmol) in DMF (5 mL) and the reaction was stirred at room temperature for 20 h. The reaction mixture was diluted with DCM and water and the layers were separated. The aqueous layer was extracted with DCM, and the combined organic layers were dried through a hydrophobic frit and concentrated in vacuo. The crude product was purified by column chromatography (eluting with 0 to 50% EtOAc in heptane) to provide the title compound (517 mg).

¹H NMR (400 MHz, DMSO-d₆) δ 9.65 (s, 1H), 9.01 (d, J=4.4 Hz, 1H), 7.67 (d, J=4.4 Hz, 1H), 4.39-4.27 (m, 1H), 3.14-3.01 (m, 2H), 2.83-2.68 (m, 2H) LCMS (Method 1): 1.21 min, 354.0, 356.0 [M−H]⁻

### Synthesis of 2-bromo-N7-(3,3-difluorocyclobutyl)pyrazolo[1,5-a]pyrimidine-3,7-dicarboxamide

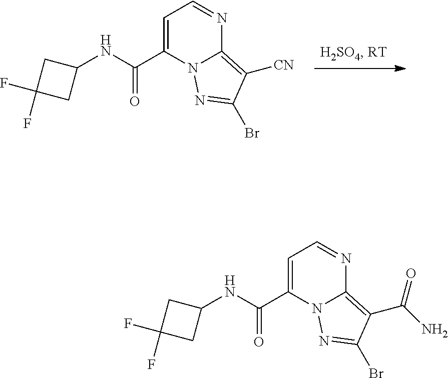 [SMILES: N#Cc1c(Br)nn2c(C(=O)NC3CC(F)(F)C3)ccnc12 ; NC(=O)c1c(Br)nn2c(C(=O)NC3CC(F)(F)C3)ccnc12]

2-Bromo-3-cyano-N-(3,3-difluorocyclobutyl)pyrazolo[1,5-a]pyrimidine-7-carboxamide (278 mg, 0.78 mmol) was dissolved in conc. H₂SO₄ (770 μL, 14.36 mmol) and the reaction was stirred at room temperature for 1.5 h. The reaction mixture was cooled to 0° C. and diluted with ice-cold water. The mixture was basified with NH₃ (28-30 wt % in water) and the resulting precipitate was collected by filtration, washed with water, and dried to provide the title compound (299 mg).

¹H NMR (400 MHz, DMSO-d₆) δ 9.74 (s, 1H), 8.94 (d, J=4.4 Hz, 1H), 7.68 (s, 2H), 7.58 (d, J=4.4 Hz, 1H), 4.44-4.27 (m, 1H), 3.20-3.01 (m, 2H), 2.85-2.67 (m, 2H)

LCMS (Method 1): 0.99 min, 374.0, 376.0 [M+H]⁺

### Synthesis of Compound 34, N7-(3,3-difluorocyclobutyl)-2-(methylamino)pyrazolo[1,5-a]pyrimidine-3,7-dicarboxamide

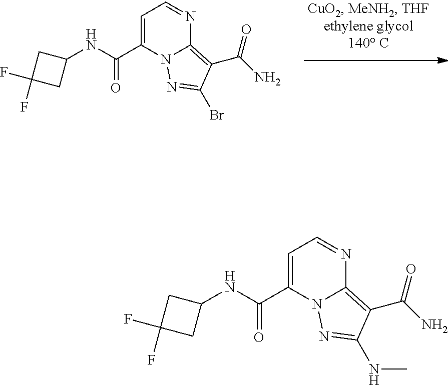 [SMILES: NC(=O)c1c(Br)nn2c(C(=O)NC3CC(F)(F)C3)ccnc12 ; CNc1nn2c(C(=O)NC3CC(F)(F)C3)ccnc2c1C(N)=O]

2-Bromo-N7-(3,3-difluorocyclobutyl)pyrazolo[1,5-a]pyrimidine-3,7-dicarboxamide (100 mg, 0.27 mmol), Cu₂O (4 mg, 0.03 mmol), and MeNH₂ (2 M in THF; 4 mL, 8.02 mmol) were suspended in ethylene glycol (1.1 mL) and the reaction was heated at 140° C. in a microwave reactor for 4 h. The reaction mixture was cooled to room temperature and diluted with MeOH. The mixture was filtered through a short pad of Dicalite® and washed with MeOH and DCM. The filtrate was concentrated in vacuo and the crude product was purified by column chromatography (eluting with 0 to 5% MeOH in DCM) to provide the title compound (12 mg).

¹H NMR (400 MHz, CDCl₃) δ 11.04 (d, J=6.6 Hz, 1H), 8.53 (d, J=4.6 Hz, 1H), 7.69 (s, 1H), 7.65 (d, J=4.6 Hz, 1H), 6.92 (s, 1H), 5.52 (s, 1H), 4.62-4.47 (m, 1H), 3.25-3.12 (m, 2H), 3.10 (s, 3H), 2.74-2.58 (m, 2H)

LCMS (Method 2): 2.06 min, 325.2 [M+H]⁺

### Synthesis of Compound 35, N7-(3,3-difluorocyclobutyl)-2-(dimethylamino)pyrazolo[1,5-a]pyrimidine-3,7-dicarboxamide

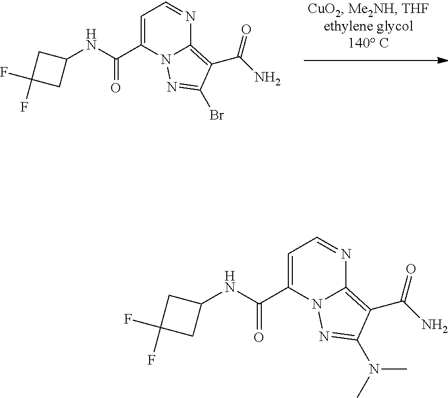 [SMILES: NC(=O)c1c(Br)nn2c(C(=O)NC3CC(F)(F)C3)ccnc12 ; CN(C)c1nn2c(C(=O)NC3CC(F)(F)C3)ccnc2c1C(N)=O]

2-Bromo-N7-(3,3-difluorocyclobutyl)pyrazolo[1,5-a]pyrimidine-3,7-dicarboxamide (50 mg, 0.13 mmol), Cu₂O (1.9 mg, 0.01 mmol), and Me₂NH (2 M in THF; 2 mL, 4.01 mmol) were suspended in ethylene glycol (530 μL) and the reaction was heated at 140° C. in a microwave reactor for 4 h. The reaction mixture was cooled to room temperature and diluted with MeOH. The mixture was filtered through a short pad of Dicalite® and washed with MeOH and DCM. The filtrate was concentrated in vacuo and the crude product was purified twice by column chromatography (eluting with 0 to 5% MeOH in DCM and then with 0 to 3% MeOH in DCM) to provide the title compound (23 mg).

¹H NMR (400 MHz, DMSO-d₆) δ 10.39 (d, J=5.7 Hz, 1H), 8.70 (d, J=4.5 Hz, 1H), 7.91 (s, 1H), 7.49 (d, J=4.5 Hz, 1H), 7.31 (s, 1H), 4.45-4.32 (m, 1H), 3.20-3.07 (m, 8H), 2.79-2.63 (m, 2H)

LCMS (Method 2): 2.04 min, 339.2 [M+H]⁺

### Synthesis of ethyl 3-(2-fluorophenyl) prop-2-ynoate

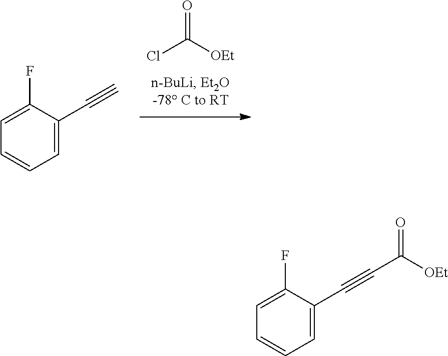

n-BuLi (2.5 M in hexanes; 7.33 mL, 18.32 mmol) was added slowly to a solution of 1-ethynyl-2-fluoro-benzene (1.89 mL, 16.65 mmol) in Et₂O (30 mL) at −78° C. and the reaction was stirred at −78° C. for 30 min. Ethyl chloroformate (1.9 mL, 19.98 mmol) was added, and the reaction was warmed to room temperature and stirred for 2 h. The reaction mixture was cooled to 0° C. and quenched with sat. aq. NH₄Cl. The layers were separated, and the aqueous layer was extracted with EtOAc (×2). The combined organic layers were washed with brine, dried over Na₂SO₄, and concentrated in vacuo. The crude product was purified by column chromatography (eluting with 0 to 40% EtOAc in heptane) to provide the title compound (2.07 g).

¹H NMR (400 MHz, CDCl₃) δ 7.61-7.51 (m, 1H), 7.49-7.39 (m, 1H), 7.20-7.05 (m, 2H), 4.30 (q, J=7.1 Hz, 2H), 1.35 (t, J=7.1 Hz, 3H) LCMS (Method 1): 1.85 min (product does not ionise)

### Synthesis of ethyl 2-(2-fluorophenyl)pyrazolo[1,5-a]pyridine-3-carboxylate

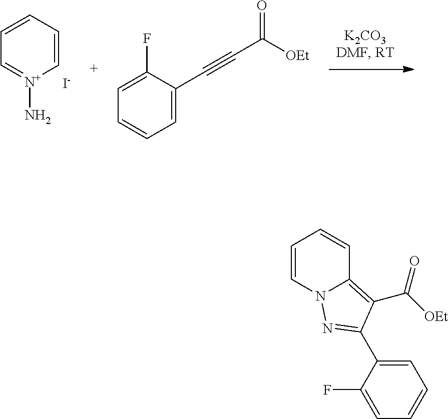 [SMILES: N[n+]1ccccc1 ; [I-] ; CCOC(=O)C#Cc1ccccc1F ; CCOC(=O)c1c(-c2ccccc2F)nn2ccccc12]

Pyridin-1-ium-1-amine iodide (1.73 g, 7.80 mmol), ethyl 3-(2-fluorophenyl) prop-2-ynoate (1.5 g, 7.80 mmol), and K₂CO₃ (1.62 g, 11.71 mmol) were dissolved in DMF (7 mL) and the reaction was stirred at room temperature overnight. The reaction mixture was poured into water and diluted with EtOAc. The layers were separated, and the aqueous layer was extracted with EtOAc (×2). The combined organic layers were dried over Na₂SO₄ and concentrated in vacuo. The crude product was purified by column chromatography (eluting with 0 to 30% EtOAc in heptane) to provide the title compound (584 mg).

¹H NMR (400 MHz, DMSO-d₆) δ 8.90 (dt, J=6.9, 1.1 Hz, 1H), 8.13 (dt, J=8.9, 1.2 Hz, 1H), 7.65 (ddd, J=8.9, 6.9, 1.1 Hz, 1H), 7.60-7.49 (m, 2H), 7.36-7.26 (m, 2H), 7.21 (td, J=6.9, 1.4 Hz, 1H), 4.16 (q, J=7.1 Hz, 2H), 1.14 (t, J=7.1 Hz, 3H)

LCMS (Method 1): 1.84 min, 285.2 [M+H]⁺

### Synthesis of ethyl 7-bromo-2-(2-fluorophenyl)pyrazolo[1,5-a]pyridine-3-carboxylate

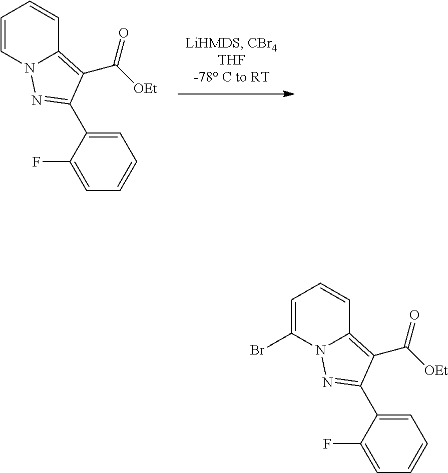 [SMILES: CCOC(=O)c1c(-c2ccccc2F)nn2ccccc12 ; CCOC(=O)c1c(-c2ccccc2F)nn2c(Br)cccc12]

LiHMDS (1 M in THF; 2.56 mL, 2.56 mmol) was added to a solution of ethyl 2-(2-fluorophenyl)pyrazolo[1,5-a]pyridine-3-carboxylate (560 mg, 1.97 mmol) in Et₂O (20 mL) at −78° C. and the mixture was stirred at −78° C. for 30 min. Carbon tetrabromide (979 mg, 2.95 mmol) was added in one portion, and the reaction was warmed to room temperature and stirred for 1.5 h. The reaction mixture was cooled to 0° C. and quenched by the slow addition of sat. aq. NH₄Cl. The layers were separated, and the aqueous layer was extracted with EtOAc (×2). The combined organic layers were washed with brine, dried over Na₂SO₄, and concentrated in vacuo. The crude product was purified by column chromatography (eluting with 0 to 40% EtOAc in heptane) to provide the title compound (439 mg).

¹H NMR (400 MHz, DMSO-d₆) δ 8.19 (dd, J=8.7, 1.3 Hz, 1H), 7.65 (dd, J=7.4, 1.3 Hz, 1H), 7.60-7.54 (m, 3H), 7.39-7.29 (m, 2H), 4.18 (q, J=7.1 Hz, 2H), 1.15 (t, J=7.1 Hz, 3H)

LCMS (Method 1): 1.98 min, 363.0, 365.2 [M+H]⁺

### Synthesis of ethyl 2-(2-fluorophenyl)-7-(indan-2-ylcarbamoyl)pyrazolo[1,5-a]pyridine-3-carboxylate

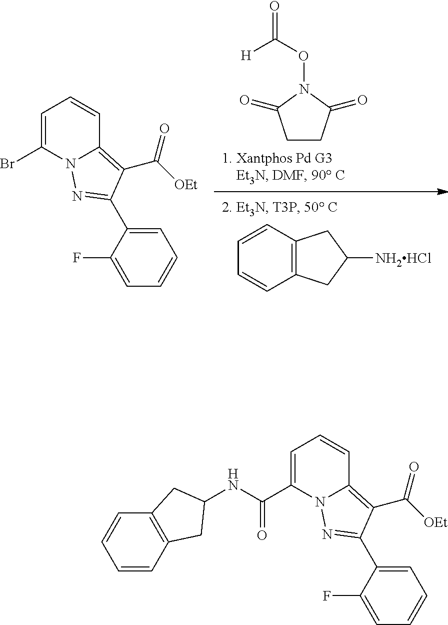

Ethyl 7-bromo-2-(2-fluorophenyl)pyrazolo[1,5-a]pyridine-3-carboxylate (400 mg, 1.10 mmol) was dissolved in DMF (4 mL) and the solution was degassed by sparging with a stream of argon. Et₃N (307 μL, 2.20 mmol), XantPhos Pd G3 (52 mg, 0.06 mmol), and (2,5-dioxopyrrolidin-1-yl) formate (472 mg, 3.30 mmol) were added, and the vial was sealed immediately. The reaction was heated at 90° C. for 4 h then cooled to room temperature. Indan-2-amine hydrochloride (186 mg, 1.10 mmol), Et₃N (307 μL, 2.20 mmol), and 1-propylphosphonic anhydride (50 wt % in EtOAc; 1.31 mL, 2.20 mmol) were added, and the reaction was heated at 50° C. for 18 h. The reaction mixture was cooled to room temperature and diluted with EtOAc and water. The layers were separated, and the aqueous layer was extracted with EtOAc (×2). The combined organic layers were washed with 5% aq. LiCl (×5), dried over Na₂SO₄, and concentrated in vacuo. The crude product was purified by column chromatography (eluting with 0 to 60% EtOAc in heptane) to provide the title compound (200 mg).

¹H NMR (400 MHz, CDCl₃) δ 10.92 (d, J=7.2 Hz, 1H), 8.42 (dd, J=8.9, 1.5 Hz, 1H), 8.16 (dd, J=7.3, 1.5 Hz, 1H), 7.65-7.54 (m, 1H), 7.53-7.39 (m, 1H), 7.41-7.35 (m, 1H), 7.27-7.09 (m, 5H), 4.95-4.87 (m, 1H), 4.28 (q, J=7.1 Hz, 2H), 3.43 (dd, J=15.9, 7.0 Hz, 2H), 2.99 (dd, J=15.9, 5.3 Hz, 2H), 1.23 (t, J=7.1 Hz, 4H) LCMS (Method 1): 2.10 min, 444.2 [M+H]⁺

### Synthesis of 2-(2-fluorophenyl)-7-indan-2-ylcarbamoyl)pyrazolo[1,5-a]pyridine-3-carboxylic Acid

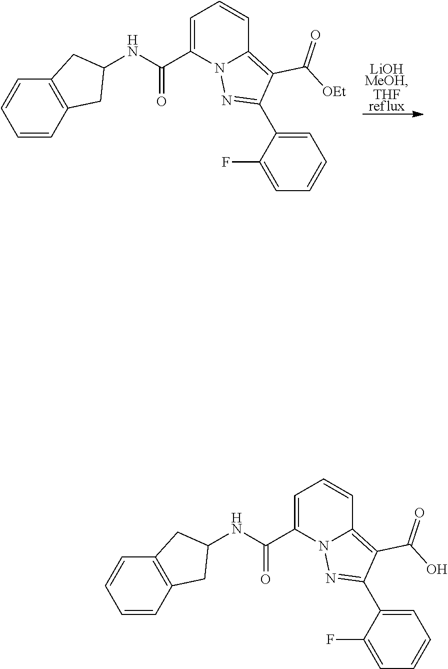 [SMILES: CCOC(=O)c1c(-c2ccccc2F)nn2c(C(=O)NC3Cc4ccccc4C3)cccc12 ; "LiOHMeOH,THFreflux" ; O=C(O)c1c(-c2ccccc2F)nn2c(C(=O)NC3Cc4ccccc4C3)cccc12]

2 M aq. LiOH (230 μL, 0.45 mmol) was added to a solution of ethyl 2-(2-fluorophenyl)-7-(indan-2-ylcarbamoyl)pyrazolo[1,5-a]pyridine-3-carboxylate (200 mg, 0.45 mmol) in THF (6 mL) and MeOH (3 mL), and the reaction was heated at reflux overnight. The reaction mixture was cooled to room temperature and concentrated in vacuo. The residue was dissolved in water and TBME, and the layers were separated. The aqueous layer was acidified to pH 2 with 5 M aq. HCl and extracted with a 1:9 mixture of MeOH:DCM (×3). The combined organic layers were dried over Na₂SO₄ and concentrated in vacuo to provide the title compound (185 mg).

¹H NMR (400 MHz, DMSO-d₆) δ 10.26 (d, J=7.2 Hz, 1H), 8.32 (dd, J=8.8, 1.5 Hz, 1H), 7.81 (dd, J=7.2, 1.5 Hz, 1H), 7.72 (dd, J=8.8, 7.2 Hz, 1H), 7.58-7.49 (m, 1H), 7.41 (td, J=7.4, 1.8 Hz, 1H), 7.35-7.26 (m, 2H), 7.23 (dd, J=5.4, 3.4 Hz, 2H), 7.19-7.11 (m, 2H), 4.85-4.68 (m, 1H), 2.91 (dd, J=16.0, 4.8 Hz, 2H). Exchangeable proton not observed, 2H obscured by solvent peak.

LCMS (Method 1): 1.84 min, 416.2 [M+H]⁺

### Synthesis of Compound 36, 2-(2-fluorophenyl)-N7-indan-2-yl-pyrazolo[1,5-a]pyridine-3,7-dicarboxamide

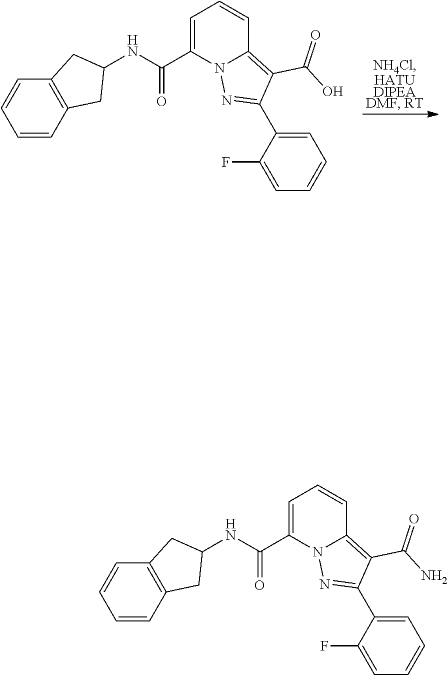 [SMILES: O=C(O)c1c(-c2ccccc2F)nn2c(C(=O)NC3Cc4ccccc4C3)cccc12 ; "NH4Cl,HATUDIPEADMF, RT" ; NC(=O)c1c(-c2ccccc2F)nn2c(C(=O)NC3Cc4ccccc4C3)cccc12]

2-(2-Fluorophenyl)-7-(indan-2-ylcarbamoyl)pyrazolo[1,5-a]pyridine-3-carboxylic acid (100 mg, 0.24 mmol), HATU (138 mg, 0.36 mmol), DIPEA (130 μL, 0.72 mmol), and NH₄Cl (39 mg, 0.72 mmol) were dissolved in DMF (2 mL) and the reaction was stirred at room temperature overnight. The reaction mixture was poured into water and stirred for 30 min, and the resulting precipitate was collected by filtration and dried. The crude product was purified by preparative HPLC (basic method) to provide the title compound (35 mg).

¹H NMR (400 MHz, DMSO-d₆) δ 10.54 (d, J=7.2 Hz, 1H), 8.11 (dd, J=8.9, 1.5 Hz, 1H), 7.83 (dd, J=7.2, 1.5 Hz, 1H), 7.64-7.48 (m, 2H), 7.40 (td, J=7.5, 1.7 Hz, 1H), 7.36-7.23 (m, 5H), 7.22-7.15 (m, 2H), 4.85-4.77 (m, 1H), 2.94 (dd, J=16.0, 4.4 Hz, 2H). Additional exchangeable proton not observed, 2H obscured by solvent peak.

LCMS (Method 2): 2.43 min, 415.2 [M+H]⁺

### Synthesis of 2-(tetrahydrofuran-3-carbonyl)propanedinitrile

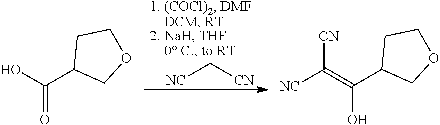 [SMILES: "1. (COCl)2, DMF    DCM, RT2. NaH, THF    0° C., to RT" ; O=C(O)C1CCOC1 ; N#CCC#N ; N#CC(C#N)=C(O)C1CCOC1]

DMF (167 μL, 2.15 mmol) was added to a solution of tetrahydrofuran-3-carboxylic acid (2.1 mL, 21.53 mmol) in DCM (50 mL), followed by the dropwise addition of oxalyl chloride (2.2 mL, 25.84 mmol) over 1 h. The reaction was stirred at room temperature for 1 h then concentrated in vacuo. NaH (1.66 g, 41.62 mmol) was suspended in THF (10 mL) and the mixture was cooled to 0° C. A solution of propanedinitrile (1.37 g, 20.81 mmol) in THF (10 mL) was added dropwise over 15 min and the reaction was stirred at room temperature for a further 15 min. The reaction mixture was cooled back to 0° C. and a solution of tetrahydrofuran-3-carbonyl chloride (formed as above, 2.8 g, 20.81 mmol) in THF (10 mL) was added dropwise over 15 min. The reaction was stirred at room temperature for 24 h then quenched with water. The mixture was acidified with 1 M aq. HCl and diluted with EtOAc. The layers were separated, and the organic layer was washed with brine, dried over Na₂SO₄, and concentrated in vacuo. The crude product was purified by column chromatography (eluting with 0 to 10% MeOH in DCM) to provide the title compound (472 mg).

¹H NMR (400 MHz, MeOD) δ 3.98-3.90 (m, 1H), 3.89-3.82 (m, 1H), 3.80-3.69 (m, 2H), 3.65-3.56 (m, 1H), 2.13-2.02 (m, 2H). Exchangeable proton not observed.

LCMS (Method 2): 1.36 min, 163.2 [M−H]⁻

### Synthesis of 2-[methoxy(tetrahydrofuran-3-yl)methylene]propanedinitrile

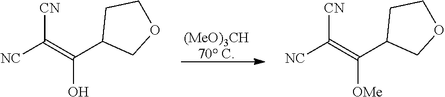 [SMILES: N#CC(C#N)=C(O)C1CCOC1 ; "(MeO)3CH70° C." ; COC(=C(C#N)C#N)C1CCOC1]

2-[Hydroxy (tetrahydrofuran-3-yl)methylene]propanedinitrile (735 mg, 4.48 mmol) was dissolved in trimethoxymethane (8 mL, 73.12 mmol) and the reaction was heated at 70° C. for 15 h. The reaction mixture was cooled to room temperature and concentrated in vacuo.

The crude product was purified by column chromatography (eluting with 0 to 40% EtOAc in heptane) to provide the title compound (492 mg).

¹H NMR (400 MHz, CDCl₃) δ 4.40 (s, 3H), 3.97-3.87 (m, 2H), 3.87-3.76 (m, 2H), 3.72-3.63 (m, 1H), 2.30-2.20 (m, 1H), 2.10-1.99 (m, 1H)

LCMS (Method 2): 1.66 min, 177.0 [M−H]⁻

### Synthesis of 5-amino-3-tetrahydrofuran-3-yl-1H-pyrazole-4-carbonitrile

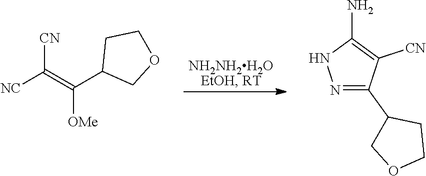

Hydrazine monohydrate (230 μL, 4.72 mmol) was added to a solution of 2-[methoxy(tetrahydrofuran-3-yl)methylene]propanedinitrile (488 mg, 2.74 mmol) in EtOH (5 mL) and the reaction was stirred at room temperature for 1.5 h then concentrated in vacuo. The crude product was purified by column chromatography (eluting with 0 to 100% EtOAc in DCM) to provide the title compound (302 mg).

¹H NMR (400 MHz, DMSO-d₆) δ 11.80 (s, 1H), 6.15 (s, 2H), 3.96 (t, J=7.9 Hz, 1H), 3.86-3.69 (m, 2H), 3.69-3.61 (m, 1H), 3.29 (d, J=7.6 Hz, 1H), 2.29-2.11 (m, 1H), 2.09-1.98 (m, 1H)

LCMS (Method 2): 1.28 min, 179.0 [M+H]⁺

### Synthesis of ethyl 3-cyano-2-tetrahydrofuran-3-yl-pyrazolo[1,5-a]pyrimidine-7-carboxylate

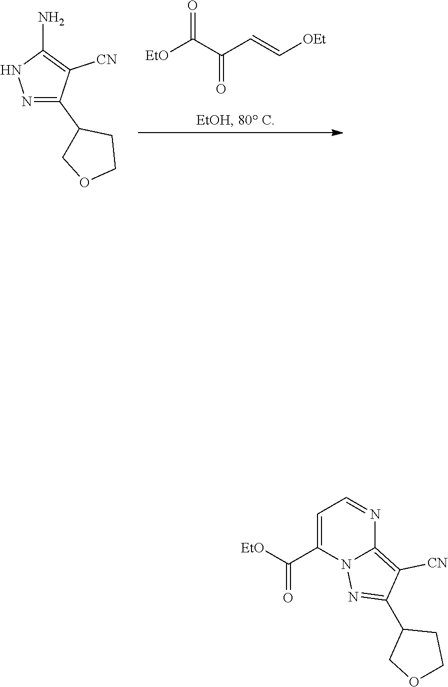

5-Amino-3-tetrahydrofuran-3-yl-1H-pyrazole-4-carbonitrile (302 mg, 1.69 mmol) and ethyl (E)-4-ethoxy-2-oxo-but-3-enoate (195 mg, 1.13 mmol) were dissolved in EtOH (7 mL) and the reaction was heated at 80° C. for 5 h. The reaction mixture was cooled to room temperature and concentrated in vacuo. The crude product was purified by column chromatography (eluting with 0 to 5% MeOH in DCM) to provide the title compound (225 mg).

¹H NMR (400 MHz, CDCl₃) δ 8.78 (d, J=4.3 Hz, 1H), 7.45 (d, J=4.2 Hz, 1H), 4.57 (q, J=7.1 Hz, 2H), 4.32-4.24 (m, 1H), 4.18-3.96 (m, 3H), 3.95-3.84 (m, 1H), 2.58-2.36 (m, 2H), 1.48 (t, J=7.1 Hz, 3H)

LCMS (Method 2): 2.05 min, 287.2 [M+H]⁺

### Synthesis of 3-cyano-2-tetrahydrofuran-3-yl-pyrazolo[1,5-a]pyrimidine-7-carboxylic Acid

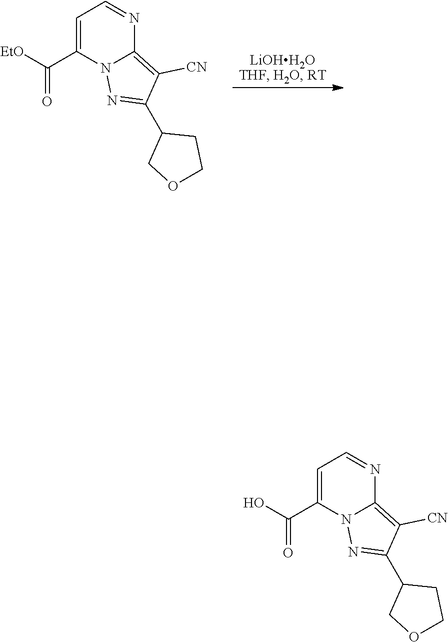

A solution of LiOH·H₂O (36 mg, 0.86 mmol) in water (2 mL) was added to a solution of ethyl 3-cyano-2-tetrahydrofuran-3-yl-pyrazolo[1,5-a]pyrimidine-7-carboxylate (225 mg, 0.79 mmol) in THF (2 mL) and the reaction was stirred at room temperature for 2 h then concentrated in vacuo. The residue was dissolved in EtOAc and water, and the layers were separated. The aqueous layer was acidified to pH 2 with 1 M aq. HCl and extracted with EtOAc. The combined organic layers were dried over Na₂SO₄ and concentrated in vacuo to provide the title compound (279 mg).

¹H NMR (400 MHz, CDCl₃) δ 8.98 (d, J=4.3 Hz, 1H), 7.96 (d, J=4.3 Hz, 1H), 4.34-4.24 (m, 1H), 4.21-4.10 (m, 1H), 4.09-3.85 (m, 4H), 2.66-2.51 (m, 1H). Exchangeable proton not observed.

LCMS (Method 2): 1.50 min, 259.2 [M+H]⁺

### Synthesis of 3-cyano-N-indan-2-yl-2-tetrahydrofuran-3-yl-pyrazolo[1,5-a]pyrimidine-7-carboxamide

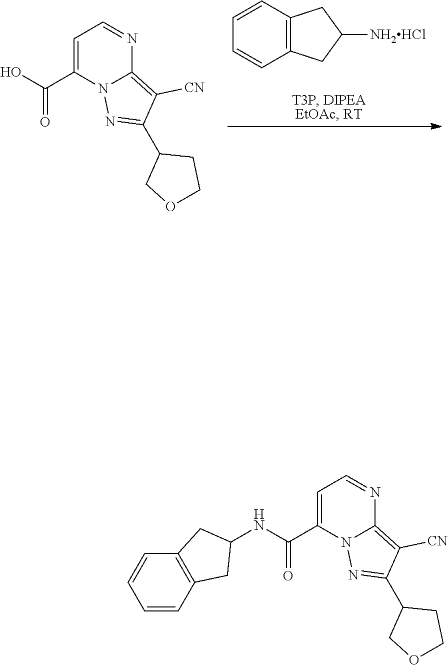

1-Propanephosphonic anhydride (50 wt % in EtOAc; 650 μL, 1.08 mmol) was added to a suspension of 3-cyano-2-tetrahydrofuran-3-yl-pyrazolo[1,5-a]pyrimidine-7-carboxylic acid (186 mg, 0.72 mmol), indan-2-amine hydrochloride (147 mg, 0.87 mmol), and DIPEA (380 μL, 2.17 mmol) in EtOAc (7 mL) and the reaction was stirred at room temperature for 3 days. The reaction mixture was diluted with EtOAc and washed with brine. The organic layer was dried over Na₂SO₄ and concentrated in vacuo to provide the title compound (431 mg). The crude product was taken forward to the next step without further purification.

¹H NMR (400 MHz, CDCl₃) δ 8.86 (d, J=4.4 Hz, 1H), 7.99 (d, J=4.4 Hz, 1H), 7.33-7.19 (m, 4H), 5.11-4.90 (m, 1H), 4.20-4.07 (m, 1H), 3.88-3.70 (m, 4H), 3.48-3.42 (m, 2H), 3.04-2.99 (m, 2H), 2.40-2.25 (m, 1H), 2.12-1.91 (m, 1H). Exchangeable proton not observed.

LCMS (Method 2): 2.43 min, 374.2 [M+H]⁺

### Synthesis of Compound 37, N7-indan-2-yl-2-tetrahydrofuran-3-yl-pyrazolo[1,5-a]pyrimidine-3,7-dicarboxamide

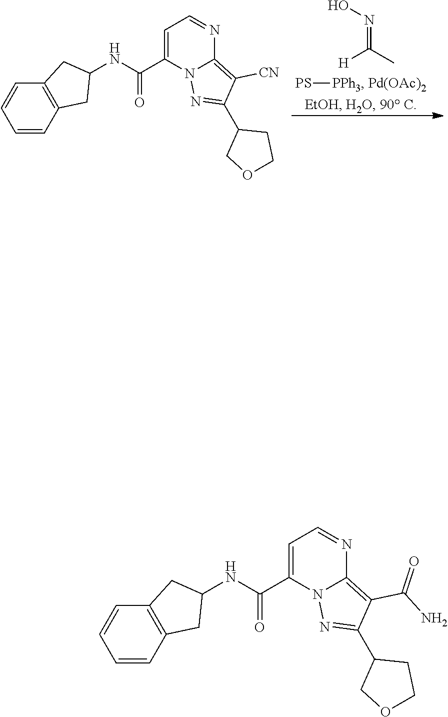

3-Cyano-N-indan-2-yl-2-tetrahydrofuran-3-yl-pyrazolo[1,5-a]pyrimidine-7-carboxamide (276 mg, 0.74 mmol), acetaldehyde oxime (180 μL, 2.96 mmol), Pd(OAc)₂ (25 mg, 0.11 mmol), and PS-PPh₃ (1.4-2 mmol/g; 130 mg, 0.18-0.26 mmol) were suspended in EtOH (6 mL) and water (1.5 mL) and the mixture was degassed by sparging with a stream of argon for 5 min. The reaction was heated at 90° C. for 18 h, then cooled to room temperature, filtered through a short pad of Dicalite®, and concentrated in vacuo. The crude product was purified by preparative HPLC (basic method) to provide the title compound (16 mg).

¹H NMR (400 MHz, DMSO-d₆) δ 10.28 (d, J=7.3 Hz, 1H), 8.90 (d, J=4.4 Hz, 1H), 7.72 (d, J=4.4 Hz, 2H), 7.54 (s, 1H), 7.35-7.26 (m, 2H), 7.24-7.15 (m, 2H), 4.88-4.77 (m, 1H), 4.34-4.23 (m, 1H), 4.07 (dd, J=8.3, 7.5 Hz, 1H), 3.79-3.65 (m, 2H), 3.56 (dd, J=8.3, 6.2 Hz, 1H), 3.38 (dd, J=16.2, 6.7 Hz, 2H), 2.96 (dd, J=16.0, 3.4 Hz, 2H), 2.17-2.05 (m, 1H), 2.03-1.92 (m, 1H)

LCMS (Method 2): 2.18 min, 392.2 [M+H]⁺

### Synthesis of ethyl (E)-4-ethoxy-3-methyl-2-oxo-but-3-enoate

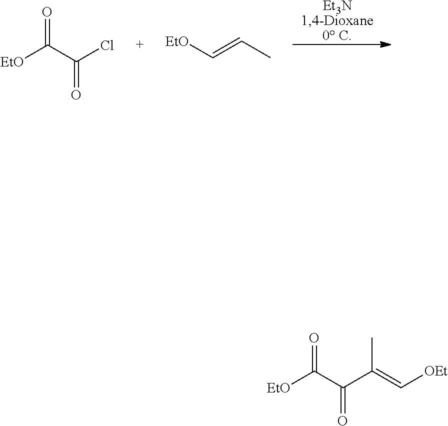

(E)-1-Ethoxyprop-1-ene (8.1 mL, 73.24 mmol) was added to a solution of Et₃N (2.45 mL, 17.58 mmol) in 1,4-dioxane (4 mL) and the solution was cooled to 0° C. Ethyl 2-chloro-2-oxo-acetate (1.64 mL, 14.65 mmol) was added dropwise and the reaction was warmed to room temperature and stirred overnight. The reaction mixture was diluted with DCM and washed with sat. aq. NaHCO₃ and water. The organic layer was dried over Na₂SO₄ and concentrated in vacuo to provide the title compound (2.097 g).

¹H NMR (400 MHz, DMSO-d₆) δ 7.58 (q, J=1.1 Hz, 1H), 4.30-4.24 (m, 4H), 1.61 (d, J=1.1 Hz, 3H), 1.30-1.25 (m, 6H)

LCMS (Method 2): 2.23 min, 187.2 [M+H]⁺

### Synthesis of ethyl 3-cyano-2,6-dimethyl-pyrazolo[1,5-a]pyrimidine-7-carboxylate

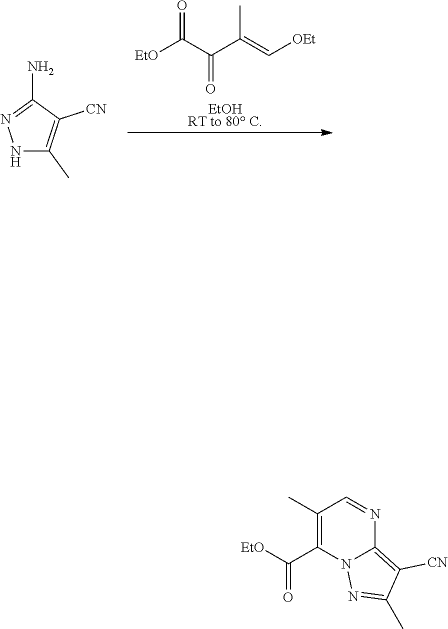

Ethyl (E)-4-ethoxy-3-methyl-2-oxo-but-3-enoate (2.01 g, 10.81 mmol) and 5-amino-3-methyl-1H-pyrazole-4-carbonitrile (1.1 g, 9.01 mmol) were dissolved in EtOH (20 mL) and the reaction was stirred at room temperature for 2 h and then heated at 80° C. for 18 h. The reaction mixture was cooled to room temperature, and the resulting precipitate was collected by filtration, washed sparingly with EtOH and dried. The filtrate was concentrated in vacuo and purified by column chromatography (eluting with 0 to 50% EtOAc in heptane). The purified product was combined with that of the solid precipitate to provide the title compound (1.414 g).

¹H NMR (400 MHz, CDCl₃) δ 8.58 (s, 1H), 4.61 (q, J=7.1 Hz, 2H), 2.61 (s, 3H), 2.42 (s, 3H), 1.47 (t, J=7.1 Hz, 3H)

LCMS (Method 2): 2.20 min, 245.0 [M+H]⁺

### Synthesis of 3-cyano-2,6-dimethyl-pyrazolo[1,5-a]pyrimidine-7-carboxylic Acid

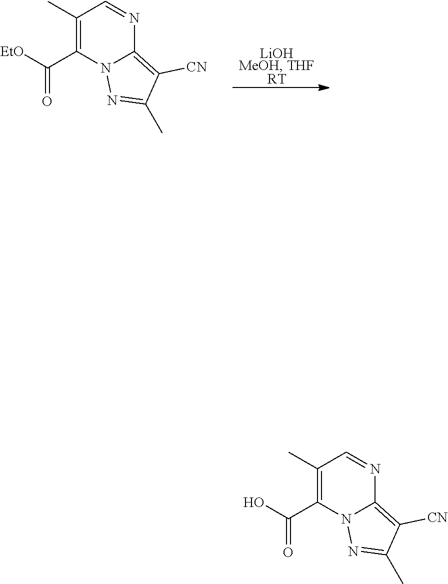

4 M aq. LiOH (614 μL, 2.46 mmol) was added to a solution of ethyl 3-cyano-2,6-dimethyl-pyrazolo[1,5-a]pyrimidine-7-carboxylate (500 mg, 2.05 mmol) in THF (10 mL) and MeOH (2 mL) and the reaction was stirred at room temperature for 16 h. The reaction mixture was concentrated in vacuo, and the residue was acidified with 5 M aq. HCl. The resulting precipitate was collected by filtration, washed with water, and dried to provide the title compound (352 mg).

¹H NMR (400 MHz, DMSO-d₆) δ 8.80 (s, 1H), 2.52 (s, 3H), 2.35 (s, 3H). Exchangeable proton not observed.

LCMS (Method 2): 1.27 min, 215.2 [M−H]⁻

### Synthesis of 3-cyano-N-(3,3-difluorocyclobutyl)-2,6-dimethyl-pyrazolo[1,5-a]pyrimidine-7-carboxamide

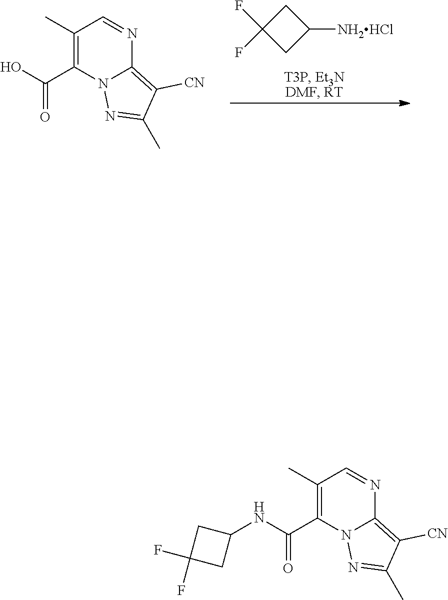

1-Propanephosphonic anhydride (50 wt % in DMF; 2.89 mL, 4.86 mmol) was added to a suspension of 3-cyano-2,6-dimethyl-pyrazolo[1,5-a]pyrimidine-7-carboxylic acid (350 mg, 1.62 mmol), 3,3-difluorocyclobutanamine hydrochloride (279 mg, 1.94 mmol), and Et₃N (680 μL, 4.86 mmol) in DMF (2.5 mL) and the reaction was stirred at room temperature for 16 h. The reaction mixture was diluted with 0.5 M aq. HCl, and the resulting precipitate was collected by filtration, washed with water and Et₂O, and dried to provide the title compound (101 mg).

¹H NMR (400 MHz, DMSO-d₆) δ 9.64 (d, J=6.2 Hz, 1H), 8.82 (s, 1H), 4.37-4.25 (m, 1H), 3.15-3.06 (m, 2H), 2.74-2.57 (m, 2H), 2.53 (s, 3H), 2.32 (d, J=3.5 Hz, 3H)

### Synthesis of Compound 38, N7-(3,3-difluorocyclobutyl)-2,6-dimethyl-pyrazolo[1,5-a]pyrimidine-3,7-dicarboxamide

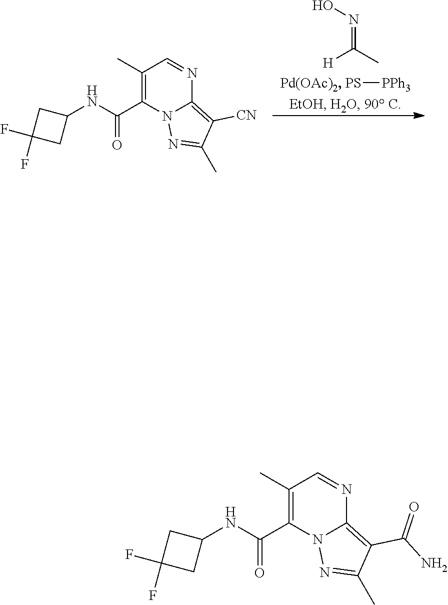

3-Cyano-N-(3,3-difluorocyclobutyl)-2,6-dimethyl-pyrazolo[1,5-a]pyrimidine-7-carboxamide (100 mg, 0.33 mmol), acetaldehyde oxime (80 μL, 1.31 mmol), Pd(OAc)₂ (11 mg, 0.05 mmol), and PS-PPh₃ (1.4-2 mmol/g; 20 mg, 0.03-0.04 mmol) were suspended in EtOH (4 mL) and water (1 mL), and the reaction was heated at 90° C. for 3 h. The reaction mixture was cooled to room temperature and concentrated in vacuo. The residue was dissolved in DCM and washed with brine. The aqueous layer was extracted with DCM, and the combined organic layers were dried over Na₂SO₄ and concentrated in vacuo. The crude product was purified by preparative HPLC (basic method) to provide the title compound (23 mg).

¹H NMR (400 MHz, DMSO-d₆) δ 9.63 (s, 1H), 8.75 (s, 1H), 7.56 (s, 1H), 7.42 (s, 1H), 4.32 (s, 1H), 3.16-3.06 (m, 2H), 2.70-2.64 (m, 2H), 2.61 (s, 3H), 2.31 (s, 3H)

LCMS (Method 2): 1.73 min, 324.2 [M+H]⁺

### Synthesis of 5-methoxy-2,4-dioxo-pentanoate

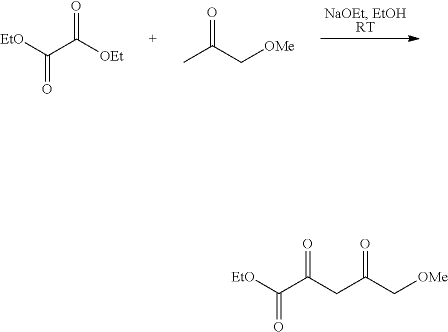

1-Methoxypropan-2-one (5 mL, 54.31 mmol) was added to a solution of NaOEt (21% in EtOH; 24.3 mL, 65.17 mmol) in EtOH (50 mL) and the mixture was stirred at room temperature for 15 min. Diethyl oxalate (8.11 mL, 59.74 mmol) was added dropwise and the reaction was stirred at room temperature for 5 days. The reaction mixture was acidified with 1 M aq. HCl and diluted with DCM. The layers were separated, and the aqueous layer was extracted with DCM. The combined organic layers were dried over Na₂SO₄ and concentrated in vacuo. The crude product was purified by vacuum distillation (17 mbar) to provide the title compound (3.6 g).

¹H NMR (400 MHz, CDCl₃) δ 6.62 (s, 1H), 4.38-4.33 (m, 2H), 4.11 (s, 2H), 3.46 (s, 3H), 1.37 (s, 3H). Exchangeable proton of enol tautomer not observed.

LCMS (Method 1): 0.97 min, 189.2 [M+H]⁺

### Synthesis of ethyl (E/Z)-4-ethoxy-5-methoxy-2-oxo-pent-3-enoate

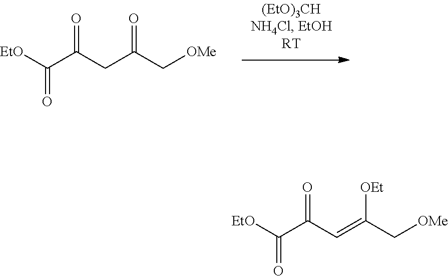 [SMILES: CCOC(=O)C(=O)CC(=O)COC ; CCOC(=O)C(=O)/C=C(/COC)OCC]

Ethyl 5-methoxy-2,4-dioxo-pentanoate (3.6 g, 19.13 mmol), triethylorthoformate (3.82 mL, 22.96 mmol), and NH₄Cl (307 mg, 5.74 mmol) were suspended in EtOH (20 mL) and the reaction was stirred at room temperature for 60 h then concentrated in vacuo. The residue was dissolved in Et₂O and the solution was filtered and concentrated in vacuo to provide the title compound (3.6 g) as a 1:1 mixture of E:Z isomers. The crude product was taken forward to the next step without purification.

LCMS (Method 1): 1.08 min, 217.2 [M+H]⁺

### Synthesis of ethyl 3-cyano-5-(methoxymethyl)-2-methyl-pyrazolo[1,5-a]pyrimidine-7-carboxylate

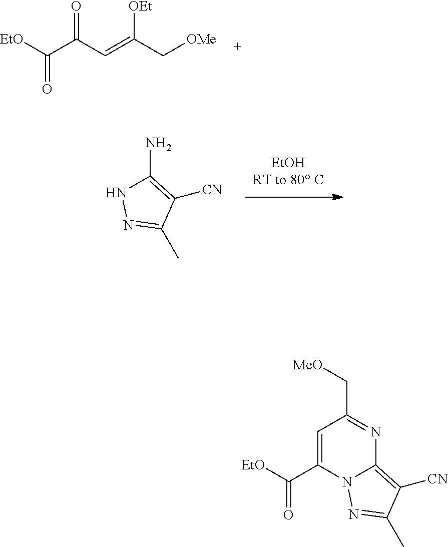

Ethyl (E/Z)-4-ethoxy-5-methoxy-2-oxo-pent-3-enoate (3.6 g, 11.65 mmol) and 5-amino-3-methyl-1H-pyrazole-4-carbonitrile (1.85 g, 15.15 mmol) were suspended in EtOH (10 mL) and the reaction was stirred at room temperature for 2 h then heated at 80° C. for 5 h. The reaction mixture was cooled to room temperature and then to 4° C. and left to settle. The resulting suspension was filtered and concentrated in vacuo. The crude product was purified by column chromatography (eluting with 0 to 100% EtOAc in heptane) to provide the title compound (2.6 g).

¹H NMR (400 MHz, CDCl₃) δ 7.62 (s, 1H), 4.67 (s, 2H), 4.57 (q, J=7.1 Hz, 2H), 3.53 (s, 3H), 2.66 (s, 3H), 1.47 (t, J=7.1 Hz, 3H)

LCMS (Method 1): 1.15 min, 275.2 [M+H]⁺

### Synthesis of 3-cyano-5-(methoxymethyl)-2-methyl-pyrazolo[1,5-a]pyrimidine-7-carboxylic Acid

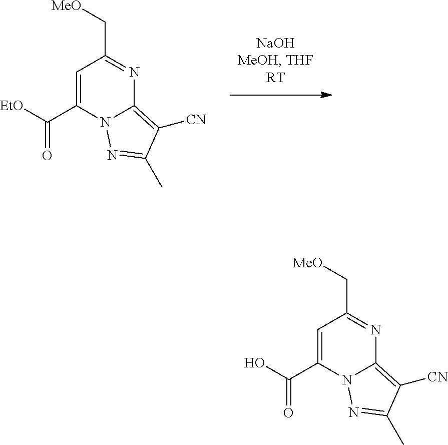 [SMILES: CCOC(=O)c1cc(COC)nc2c(C#N)c(C)nn12 ; COCc1cc(C(=O)O)n2nc(C)c(C#N)c2n1]

2 M aq. NaOH (16 mL, 32 mmol) was added to a solution of ethyl 3-cyano-5-(methoxymethyl)-2-methyl-pyrazolo[1,5-a]pyrimidine-7-carboxylate (2.6 g, 6.64 mmol) in MeOH (2 mL) and THF (18 mL) and the reaction was stirred at room temperature for 1 h then concentrated in vacuo. The residue was suspended in water and acidified to pH 2 with 5 M aq. HCl. The resulting precipitate was collected by filtration, washed with water, and dried to provide the title compound (617 mg).

¹H NMR (400 MHz, DMSO-d₆) δ 6.92 (s, 1H), 4.56 (s, 2H), 3.39 (s, 3H), 2.49 (s, 3H). Exchangeable proton not observed.

LCMS (Method 1): 0.86 min, 203.2 [M-CO₂H+H]⁺

### Synthesis of 3-cyano-N-(3,3-difluorocyclobutyl)-5-(methoxymethyl)-2-methyl-pyrazolo[1,5-a]pyrimidine-7-carboxamide

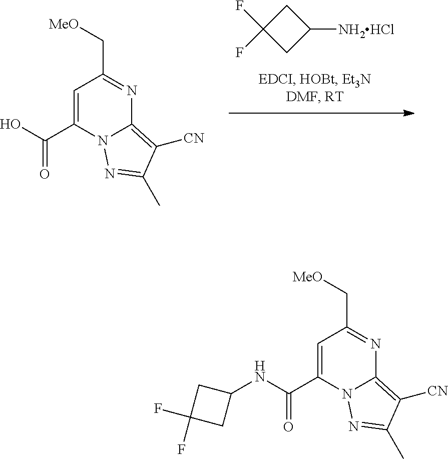

Et₃N (540 μL, 3.90 mmol) was added to a suspension of 3-cyano-5-(methoxymethyl)-2-methyl-pyrazolo[1,5-a]pyrimidine-7-carboxylic acid (160 mg, 0.65 mmol), 3,3-difluorocyclobutanamine hydrochloride (187 mg, 1.30 mmol), HOBt (299 mg, 1.95 mmol), and EDCl (374 mg, 1.95 mmol) in DMF (2 mL) and the reaction was stirred at room temperature for 72 h. The reaction mixture was diluted with EtOAc and washed with 5% aq. LiCl (×2), 1 M aq. HCl, and brine. The organic layer was dried over Na₂SO₄ and concentrated in vacuo. The crude product was purified by column chromatography (eluting with 0 to 100% EtOAc in heptane) to provide the title compound (65 mg).

¹H NMR (400 MHz, CDCl₃) δ 10.44 (s, 1H), 8.13 (s, 1H), 4.70 (s, 2H), 4.54-4.46 (m, 1H), 3.53 (s, 3H), 3.21-3.11 (m, 2H), 2.78-2.70 (m, 2H), 2.70 (s, 3H)

LCMS (Method 1) 1.24 min, 336.2 [M+H]⁺

### Synthesis of Compound 39, N7-(3,3-difluorocyclobutyl)-5-(methoxymethyl)-2-methyl-pyrazolo[1,5-a]pyrimidine-3,7-dicarboxamide

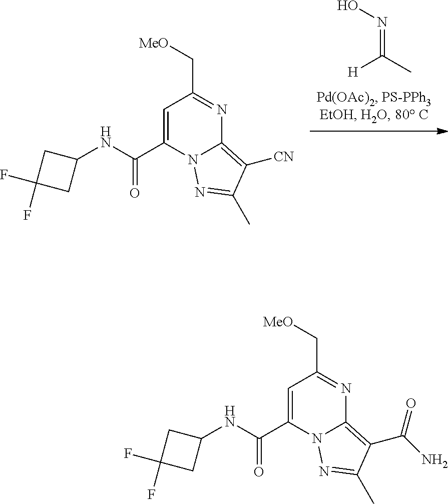

3-Cyano-N-(3,3-difluorocyclobutyl)-5-(methoxymethyl)-2-methyl-pyrazolo[1,5-a]pyrimidine-7-carboxamide (65 mg, 0.19 mmol), Pd(OAc)₂ (8.7 mg, 0.04 mmol), PS-PPh₃ (1-1.5 mmol/g; 30 mg, 0.03-0.05 mmol), and acetaldehyde oxime (59 μL, 0.97 mmol) were suspended in EtOH (2.5 mL) and water (500 μL) and the reaction was heated at 80° C. for 20 h. The reaction mixture was cooled to room temperature and concentrated in vacuo. The crude product was purified by column chromatography (eluting with 0 to 10% (2 M NH₃ in MeOH) in DCM) to provide the title compound (38 mg).

¹H NMR (400 MHz, DMSO-d₆) δ 10.17 (d, J=6.5 Hz, 1H), 7.67 (s, 1H), 7.57 (s, 1H), 7.52 (s, 1H), 4.70 (s, 2H), 4.40-4.32 (m, 1H), 3.44 (s, 3H), 3.16-3.01 (m, 2H), 2.91-2.74 (m, 2H), 2.70 (s, 3H)

LCMS (Method 2): 2.06 min, 354.2 [M+H]⁺

### Synthesis of ethyl 2-phenylpyrazolo[1,5-a]pyridine-3-carboxylate

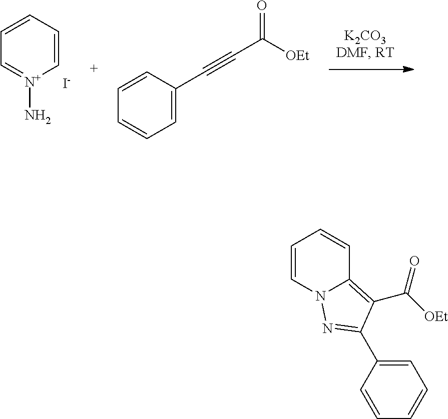 [SMILES: N[n+]1ccccc1 ; [I-] ; CCOC(=O)C#Cc1ccccc1 ; CCOC(=O)c1c(-c2ccccc2)nn2ccccc12]

Pyridin-1-ium-1-amine iodide (1 g, 4.5 mmol), ethyl 3-phenylprop-2-ynoate (1.49 mL, 9.01 mmol), and K₂CO₃ (870 mg, 6.31 mmol) were dissolved in DMF (9 mL) and the reaction was stirred at room temperature overnight. The reaction mixture was poured into water and diluted with EtOAc. The layers were separated, and the organic layer was washed with water and brine (×2), dried through a hydrophobic frit, and concentrated in vacuo. The crude product was purified by column chromatography (eluting with 0 to 30% EtOAc in heptane) to provide the title compound (696 mg).

¹H NMR (400 MHz, CDCl₃) δ 8.52 (dt, J=6.9, 1.1 Hz, 1H), 8.21 (ddd, J=9.0, 1.1 Hz, 1H), 7.85-7.76 (m, 2H), 7.49-7.43 (m, 3H), 7.40 (ddd, J=9.0, 6.9, 1.2 Hz, 1H), 6.95 (td, J=6.9, 1.4 Hz, 1H), 4.32 (q, J=7.1 Hz, 2H), 1.31 (t, J=7.1 Hz, 3H)

LCMS (Method 1): 1.87 min, 267.2 [M+H]⁺

### Synthesis of ethyl 7-bromo-2-phenyl-pyrazolo[1,5-a]pyridine-3-carboxylate

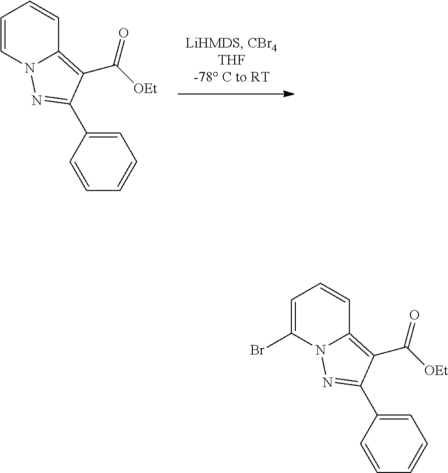 [SMILES: CCOC(=O)c1c(-c2ccccc2)nn2ccccc12 ; CCOC(=O)c1c(-c2ccccc2)nn2c(Br)cccc12]

LiHMDS (1 M in THF; 3.42 mL, 3.42 mmol) was added to a solution of ethyl 2-phenylpyrazolo[1,5-a]pyridine-3-carboxylate (700 mg, 2.63 mmol) in THF (13 mL) at −78° C. and the mixture was stirred at −78° C. for 30 min. Carbon tetrabromide (1.133 g, 3.42 mmol) was added in one portion, and the reaction was warmed to room temperature and stirred for 1.5 h. The reaction mixture was quenched by the slow addition of sat. aq. NH₄Cl and diluted with water and EtOAc. The layers were separated, and the aqueous layer was extracted with EtOAc. The combined organic layers were washed with brine, dried over Na₂SO₄, and concentrated in vacuo. The crude product was purified by column chromatography (eluting with 0 to 30% EtOAc in heptane) to provide the title compound (719 mg).

¹H NMR (400 MHz, CDCl₃) δ 8.25 (dd, J=7.8, 2.3 Hz, 1H), 7.83-7.77 (m, 2H), 7.50-7.41 (m, 3H), 7.32-7.26 (m, 2H), 4.31 (q, J=7.1 Hz, 2H), 1.29 (t, J=7.1 Hz, 3H)

LCMS (Method 1): 1.99 min, 345.1, 347.1 [M+H]⁺

### Synthesis of ethyl 7-(indan-2-ylcarbamoyl)-2-phenyl-pyrazolo[1,5-a]pyridine-3-carboxylate

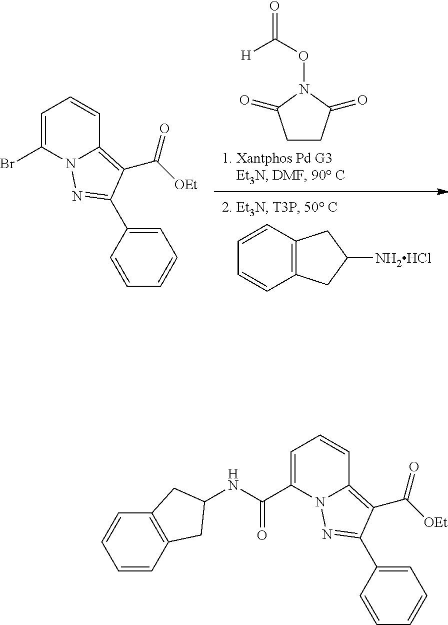

Ethyl 7-bromo-2-phenyl-pyrazolo[1,5-a]pyridine-3-carboxylate (200 mg, 0.55 mmol) and Xantphos Pd G3 (26 mg, 0.03 mmol) were placed in a vial, and the vial was sealed and purged with argon for 5 min. DMF (2 mL) and Et₃N (0.15 mL, 1.1 mmol) were added, and the mixture was degassed by sparging with argon for 5 min. (2,5-Dioxopyrrolidin-1-yl) formate (393 mg, 2.75 mmol) was added and the vial was sealed immediately. The reaction was heated at 90° C. in a microwave reactor for 1 h then cooled to room temperature. Indan-2-amine hydrochloride (140 mg, 0.83 mmol), Et₃N (0.15 mL, 1.1 mmol), and 1-propanephosphonic anhydride (50 wt % in DMF; 0.66 mL, 1.1 mmol) were added and the reaction was heated at 50° C. for 18 h. The reaction mixture was cooled to room temperature and diluted with 0.5 M aq. HCl and EtOAc. The layers were separated, and the organic layer was washed with sat. aq. NaHCO₃ and brine (×2), dried through a hydrophobic frit, and concentrated in vacuo. The crude product was purified by column chromatography (eluting with 0 to 60% EtOAc in heptane) to provide the title compound (147 mg).

¹H NMR (400 MHz, CDCl₃) δ 11.05 (d, J=7.2 Hz, 1H), 8.41 (dd, J=8.9, 1.5 Hz, 1H), 8.15 (dd, J=7.3, 1.5 Hz, 1H), 7.62-7.59 (m, 2H), 7.57 (dd, J=8.9, 7.2 Hz, 1H), 7.48-7.37 (m, 3H), 7.29-7.24 (m, 2H), 7.24-7.19 (m, 2H), 5.00 (s, 1H), 4.34 (q, J=7.1 Hz, 2H), 3.44 (dd, J=16.0, 6.9 Hz, 2H), 3.02 (dd, J=16.0, 4.6 Hz, 2H), 1.32 (t, J=7.1 Hz, 3H)

LCMS (Method 1): 2.10 min, 426.2 [M+H]⁺

### Synthesis of 7-(indan-2-ylcarbamoyl)-2-phenyl-pyrazolo[1,5-a]pyridine-3-carboxylic Acid

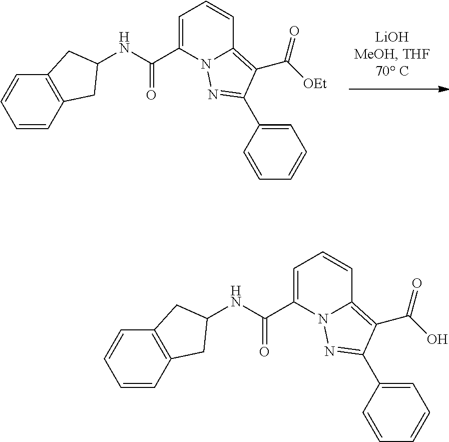 [SMILES: CCOC(=O)c1c(-c2ccccc2)nn2c(C(=O)NC3Cc4ccccc4C3)cccc12 ; O=C(O)c1c(-c2ccccc2)nn2c(C(=O)NC3Cc4ccccc4C3)cccc12]

2 M aq. LiOH (290 μL, 0.59 mmol) was added to a solution of ethyl 7-(indan-2-ylcarbamoyl)-2-phenyl-pyrazolo[1,5-a]pyridine-3-carboxylate (50 mg, 0.12 mmol) in THF (4 mL) and MeOH (2 mL) and the reaction was heated at 70° C. for 22 h. The reaction mixture was cooled to room temperature, and an additional portion of 2 M aq. LiOH (150 μL, 0.29 mmol) was added. The reaction was heated at 70° C. for 8 h, then cooled to room temperature and concentrated in vacuo. The residue was dissolved in water and TBME, and the layers were separated. The aqueous layer was acidified to pH 1-2 with 5 M aq. HCl and extracted with a 1:9 mixture of MeOH:DCM (×3). The combined organic layers were concentrated in vacuo to provide the title compound (42 mg).

¹H NMR (400 MHz, DMSO-d₆) δ 12.74 (s, 1H), 10.48 (d, J=7.2 Hz, 1H), 8.36 (dd, J=8.9, 1.5 Hz, 1H), 7.86 (dd, J=7.2, 1.5 Hz, 1H), 7.70 (dd, J=8.9, 7.3 Hz, 1H), 7.66-7.60 (m, 2H), 7.50-7.40 (m, 3H), 7.33-7.25 (m, 2H), 7.24-7.17 (m, 2H), 4.88-4.76 (m, 1H), 3.38-3.33 (m, 2H), 2.95 (dd, J=16.1, 4.0 Hz, 2H)

LCMS (Method 1): 1.83 min, 398.2 [M+H]⁺

### Synthesis of Compound 40, N7-indan-2-yl-N3-methyl-2-phenyl-pyrazolo[1,5-a]pyridine-3,7-dicarboxamide

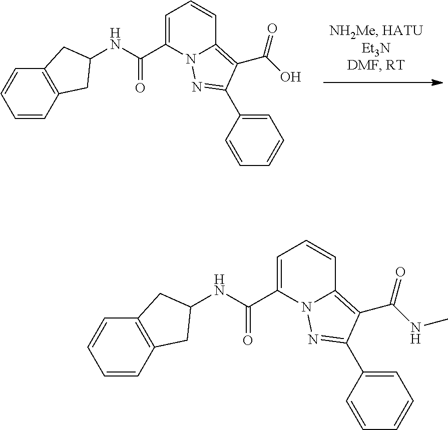 [SMILES: O=C(O)c1c(-c2ccccc2)nn2c(C(=O)NC3Cc4ccccc4C3)cccc12 ; CNC(=O)c1c(-c2ccccc2)nn2c(C(=O)NC3Cc4ccccc4C3)cccc12]

HATU (58 mg, 0.15 mmol) was added to a solution of 7-(indan-2-ylcarbamoyl)-2-phenyl-pyrazolo[1,5-a]pyridine-3-carboxylic acid (40 mg, 0.10 mmol), NH₂Me (2 M in THF; 250 UL, 0.50 mmol) and Et₃N (30 μL, 0.22 mmol) in DMF (2 mL) and the reaction was stirred at room temperature for 16 h. Additional portions of HATU (12 mg, 0.03 mmol) and NH₂Me (2 M in THF; 130 μL, 0.25 mmol) were added and the reaction was stirred at room temperature for a further 6 h. The reaction mixture was diluted with EtOAc and washed with 0.5 M aq. HCl, sat. aq. NaHCO₃, and brine (×2). The organic layer was dried through a hydrophobic frit and concentrated in vacuo. The crude product was purified by preparative HPLC (basic method) to provide the title compound (30 mg).

¹H NMR (400 MHz, DMSO-d₆) δ 10.76 (d, J=7.5 Hz, 1H), 8.12 (q, J=4.5 Hz, 1H), 7.97 (dd, J=8.9, 1.4 Hz, 1H), 7.85 (dd, J=7.2, 1.4 Hz, 1H), 7.58-7.50 (m, 3H), 7.47-7.40 (m, 3H), 7.35 (dd, J=5.4, 3.3 Hz, 2H), 7.29-7.22 (m, 2H), 4.92-4.81 (m, 1H), 3.44-3.34 (m, 2H), 2.99 (dd, J=16.1, 3.2 Hz, 2H), 2.78 (d, J=4.6 Hz, 3H)

LCMS (Method 2): 2.53 min, 411.2 [M+H]⁺

### Synthesis of 5,7-dihydroxy-2-methyl-pyrazolo[1,5-a]pyrimidine-3-carbonitrile

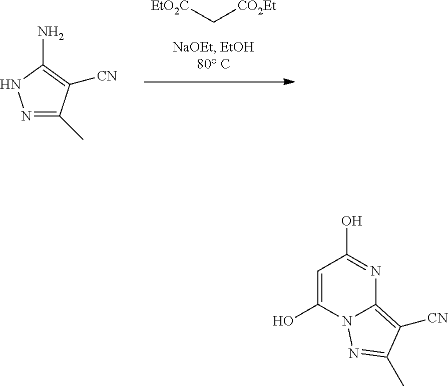

NaOEt (21 wt % in EtOH; 30.6 mL, 81.88 mmol) was added dropwise to a solution of 5-amino-3-methyl-1H-pyrazole-4-carbonitrile (5 g, 40.94 mmol) and diethyl malonate (6.87 mL, 45.03 mmol) in EtOH (125 mL) and the reaction was heated at 80° C. overnight. The reaction mixture was cooled to room temperature and the resulting precipitate was collected by filtration. The solid was suspended in water and the mixture was acidified to pH 3 with conc. HCl. The suspension was stirred at room temperature for 20 min then the solids were collected by filtration, azeotroped with MeCN, and dried to provide the title compound (6.45 g).

¹H NMR (400 MHz, DMSO-d₆) δ 5.13 (s, 1H), 2.31 (s, 3H). Exchangeable protons not observed.

LCMS (Method 1): 1.07 min, 191.2 [M+H]⁺

### Synthesis of 5,7-dichloro-2-methyl-pyrazolo[1,5-a]pyrimidine-3-carbonitrile

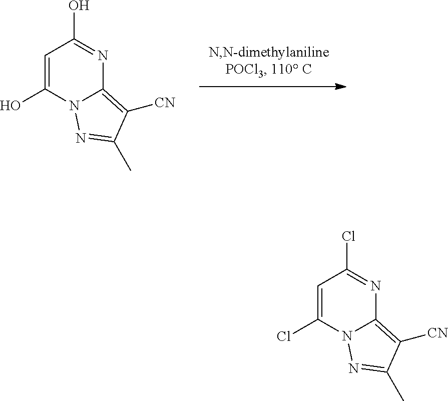

N,N-dimethylaniline (1.74 mL, 10.96 mmol) was added to a mixture of 5,7-dihydroxy-2-methyl-pyrazolo[1,5-a]pyrimidine-3-carbonitrile (1.39 g, 7.31 mmol) and POCl₃ (4.1 mL, 43.86 mmol) and the reaction was heated at 110° C. in a microwave reactor for 15 min. The reaction mixture was cooled to room temperature and diluted with DCM (10 mL). The mixture was added slowly to warm water (50 mL) with vigorous stirring, then cooled to room temperature and diluted with DCM. The biphasic mixture was dried through a hydrophobic frit and concentrated in vacuo. The crude product was purified by column chromatography (eluting with 5 to 40% EtOAc in heptane) to provide the title compound (980 mg).

¹H NMR (400 MHz, CDCl₃) δ 7.15 (s, 1H), 2.66 (s, 3H)

LCMS (Method 1): 1.64 min, 227.2, 229.2 [M+H]⁺

### Synthesis of 5-chloro-2-methyl-7-[(E)-styryl]pyrazolo[1,5-a]pyrimidine-3-carbonitrile

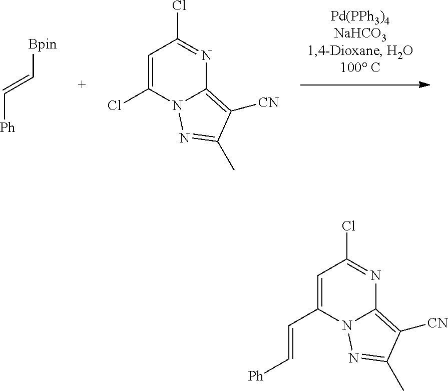

NaHCO₃ (721 mg, 8.58 mmol) and Pd(PPh₃)₄ (248 mg, 0.21 mmol) were added to a solution of 4,4,5,5-tetramethyl-2-[(E)-styryl]-1,3,2-dioxaborolane (1.18 g, 5.15 mmol) in 1,4-dioxane (5 mL) and the mixture was purged with argon for 10 min then heated to 100° C. A solution of 5,7-dichloro-2-methyl-pyrazolo[1,5-a]pyrimidine-3-carbonitrile (980 mg, 4.29 mmol) in 1,4-dioxane (4 mL) was added, followed by water (1 mL), and the reaction was heated at 100° C. for 2 h then cooled to room temperature. The reaction mixture was diluted with DCM and washed with water. The organic layer was dried through a hydrophobic frit and concentrated in vacuo. The crude product was purified by column chromatography (eluting with 0 to 100% EtOAc in heptane) to provide the title compound (916 mg).

¹H NMR (400 MHz, CDCl₃) δ 7.94 (d, J=16.5 Hz, 1H), 7.77-7.63 (m, 3H), 7.58-7.41 (m, 3H), 7.20 (s, 1H), 2.65 (s, 3H)

LCMS (Method 1): 2.01 min, 295.2, 297.2 [M+H]⁺

### Synthesis of 5-chloro-7-formyl-2-methyl-pyrazolo[1,5-a]pyrimidine-3-carbonitrile

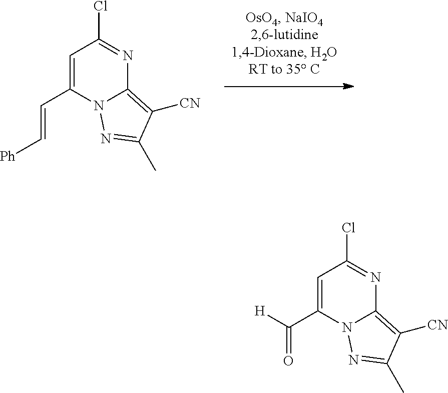 [SMILES: Cc1nn2c(/C=C/c3ccccc3)cc(Cl)nc2c1C#N ; Cc1nn2c(C=O)cc(Cl)nc2c1C#N]

2,6-Lutidine (440 μL, 3.80 mmol), followed by a solution of NaIO₄ (1.63 g, 7.60 mmol) in water (10 mL), and OsO₄ (4 wt % in water; 121 μL, 0.02 mmol) were added sequentially to a solution of 5-chloro-2-methyl-7-[(E)-styryl]pyrazolo[1,5-a]pyrimidine-3-carbonitrile (560 mg, 1.90 mmol) in 1,4-dioxane (30 mL) and the reaction was stirred at room temperature for 16 h, then heated at 35° C. for a further 16 h. The reaction mixture was cooled to room temperature and diluted with EtOAc and brine. The layers were separated, and the aqueous layer was extracted with EtOAc (×3). The combined organic layers were dried over Na₂SO₄ and concentrated in vacuo. The crude product was purified by column chromatography (eluting with 0 to 70% EtOAc in heptane) to provide the title compound (397 mg) as an impure gum. This crude product was taken forward to the next step without further purification.

LCMS (Method 1): 1.36 min, 221.2, 223.2 [M+H]⁺

### Synthesis of 5-chloro-3-cyano-2-methyl-pyrazolo[1,5-a]pyrimidine-7-carboxylic Acid

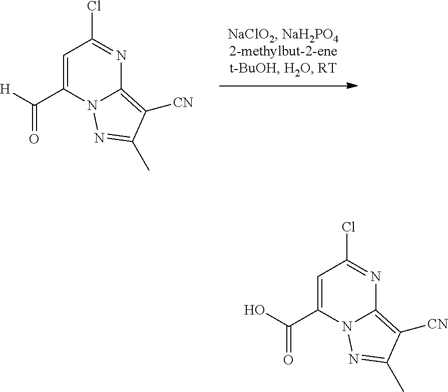 [SMILES: Cc1nn2c(C=O)cc(Cl)nc2c1C#N ; Cc1nn2c(C(=O)O)cc(Cl)nc2c1C#N]

Water (7.5 mL), NaH₂PO₄ (216 mg, 1.80 mmol), 2-methylbut-2-ene (1.53 mL, 14.40 mmol), and NaClO₂ (325 mg, 3.60 mmol) were added sequentially to a solution of the crude 5-chloro-7-formyl-2-methyl-pyrazolo[1,5-a]pyrimidine-3-carbonitrile (397 mg) in t-BuOH (15 mL) and the reaction was stirred at room temperature for 1 h. The reaction mixture was acidified to pH 3 with 1 M aq. HCl then diluted with brine. The aqueous solution was extracted with a 1:9 mixture of i-PrOH:EtOAc (×3), and the combined organic layers were dried over Na₂SO₄ and concentrated in vacuo. The crude product was purified by column chromatography (eluting with 0 to 30% MeOH in DCM) to provide the title compound (75 mg).

¹H NMR (400 MHz, MeOD) δ 7.34 (s, 1H), 2.61 (s, 3H). Exchangeable proton not observed.

LCMS (Method 2): 1.51 min, 237.2, 239.2 [M+H]⁺

### Synthesis of 5-chloro-3-cyano-N-(3,3-difluorocyclobutyl)-2-methyl-pyrazolo[1,5-a]pyrimidine-7-carboxamide

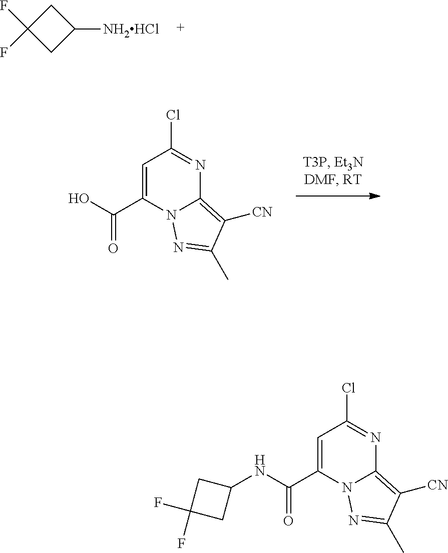

Et₃N (650 μL, 4.65 mmol), 3,3-difluorocyclobutanamine hydrochloride (160 mg, 1.12 mmol) and 1-propanephosphonic anhydride (50 wt % in DMF; 1.78 mL, 2.79 mmol) were added to a solution of 5-chloro-3-cyano-2-methyl-pyrazolo[1,5-a]pyrimidine-7-carboxylic acid (220 mg, 0.93 mmol) in DMF (5 mL) and the reaction was stirred at room temperature for 16 h. The reaction mixture was diluted with EtOAc and washed with water (×3). The combined aqueous layers were extracted with EtOAc (×3), and the combined organic layers were dried over MgSO₄ and concentrated in vacuo. The crude product was purified by column chromatography (eluting with 0 to 40% EtOAc in heptane) to provide the title compound (62 mg).

¹H NMR (400 MHz, CDCl₃) δ 10.30 (br. s, 1H), 7.89 (s, 1H), 4.55-4.44 (m, 1H), 3.25-3.10 (m, 2H), 2.80-2.64 (m, 2H), 2.61 (s, 3H)

LCMS (Method 2): 1.79 min, 324.0, 326.0 [M−H]⁻

### Synthesis of 3-cyano-N-(3,3-difluorocyclobutyl)-5-methoxy-2-methyl-pyrazolo[1,5-a]pyrimidine-7-carboxamide

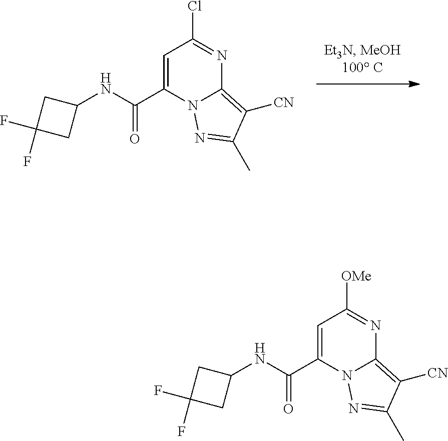 [SMILES: Cc1nn2c(C(=O)NC3CC(F)(F)C3)cc(Cl)nc2c1C#N ; COc1cc(C(=O)NC2CC(F)(F)C2)n2nc(C)c(C#N)c2n1]

Et₃N (80 μL, 0.57 mmol) was added to a solution of 5-chloro-3-cyano-N-(3,3-difluorocyclobutyl)-2-methyl-pyrazolo[1,5-a]pyrimidine-7-carboxamide (62 mg, 0.19 mmol) in MeOH (2 mL, 49.49 mmol) and the reaction was heated at 100° C. in a microwave reactor for 20 min. The reaction mixture was cooled to room temperature and dry loaded onto silica gel. The crude product was purified by column chromatography (eluting with 50 to 100% DCM in heptane) to provide the title compound (19 mg).

¹H NMR (400 MHz, CDCl₃) δ 10.44 (d, J=6.0 Hz, 1H), 7.37 (s, 1H), 4.54-4.39 (m, 1H), 4.12 (s, 3H), 3.22-3.07 (m, 2H), 2.79-2.63 (m, 2H), 2.61 (s, 3H)

LCMS (Method 1): 1.80 min, 322.2 [M+H]⁺

### Synthesis of Compound 41, N7-(3,3-difluorocyclobutyl)-5-methoxy-2-methyl-pyrazolo[1,5-a]pyrimidine-3,7-dicarboxamide

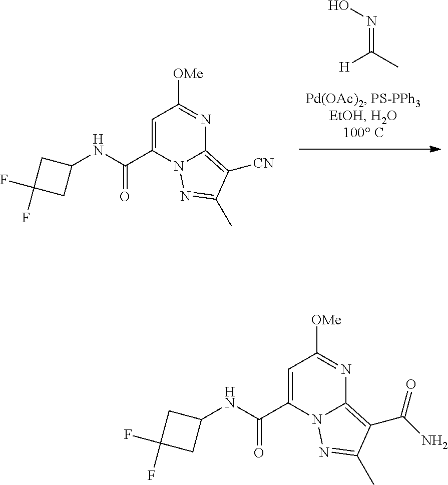

Pd(OAc)₂ (4 mg, 0.02 mmol), acetaldehyde oxime (14 mg, 0.24 mmol) and PS-PPh₃ (1.4-2 mmol/g; 9.3 mg, 0.01-0.02 mmol) were added to a solution of 3-cyano-N-(3,3-difluorocyclobutyl)-5-methoxy-2-methyl-pyrazolo[1,5-a]pyrimidine-7-carboxamide (22 mg, 0.06 mmol), in EtOH (2 mL) and water (500 μL) and the mixture was purged with argon for 5 min. The reaction was heated at 100° C. in a microwave reactor for 40 min. The reaction mixture was cooled to room temperature, filtered through a short pad of Dicalite®, and washed with MeOH. The filtrate was concentrated in vacuo, and the resulting solid was recrystallised from DMSO in EtOAc. The solid was collected by filtration, washed with EtOAc, and dried to provide the title compound (4.1 mg).

¹H NMR (400 MHz, DMSO-d₆) δ 10.09 (d, J=6.5 Hz, 1H), 7.42 (s, 1H), 7.38 (s, 1H), 7.02 (s, 1H), 4.40-4.23 (m, 1H), 4.06 (s, 3H), 3.17-2.96 (m, 2H), 2.90-2.70 (m, 2H), 2.61 (s, 3H)

LCMS (Method 2): 2.13 min, 340.0 [M+H]⁺

### Synthesis of ethyl (Z)-4-ethoxy-2-oxo-pent-3-enoate

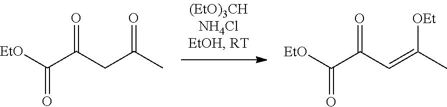

Ethyl 2,4-dioxopentanoate (5.02 g, 31.7 mmol), triethylorthoformate (6.34 mL, 38.10 mmol) and NH₄Cl (509 mg, 9.52 mmol) were suspended in EtOH (60 mL) and the reaction was stirred at room temperature for 60 h. The reaction mixture was concentrated in vacuo and Et₂O was added to the residue. The suspension was filtered and the filtrate was concentrated in vacuo. The crude product was purified by vacuum distillation (5 mBar) to provide the title compound (4.3 g).

¹H NMR (400 MHz, CDCl₃) δ 6.21 (s, 1H), 4.30 (q, J=7.2 Hz, 2H), 4.00 (q, J=7.0 Hz, 2H), 2.38 (s, 3H), 1.38 (t, J=7.0 Hz, 3H), 1.36 (t, J=7.2 Hz 3H)

### Synthesis of ethyl 3-cyano-2,5-dimethyl-pyrazolo[1,5-a]pyrimidine-7-carboxylate

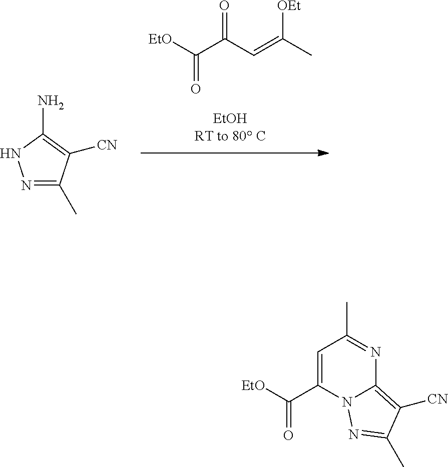

Ethyl (Z)-4-ethoxy-2-oxo-pent-3-enoate (4.3 g, 23.09 mmol) was added to a solution of 5-amino-3-methyl-1H-pyrazole-4-carbonitrile (2.80 g, 22.93 mmol) in EtOH (25 mL) and the reaction was stirred at room temperature for 1 h then heated at 80° C. for 16 h. The reaction mixture was cooled to room temperature and the resulting precipitate was collected by filtration and dried to provide the title compound (4.57 g).

¹H NMR (400 MHz, CDCl₃) δ 7.31 (s, 1H), 4.59 (q, J=7.1 Hz, 2H), 2.76 (s, 3H), 2.68 (s, 3H), 1.49 (t, J=7.1 Hz, 3H)

LCMS (Method 1): 1.08 min, 245.2 [M+H]⁺

### Synthesis of ethyl 3-carbamoyl-2,5-dimethyl-pyrazolo[1,5-a]pyrimidine-7-carboxylate

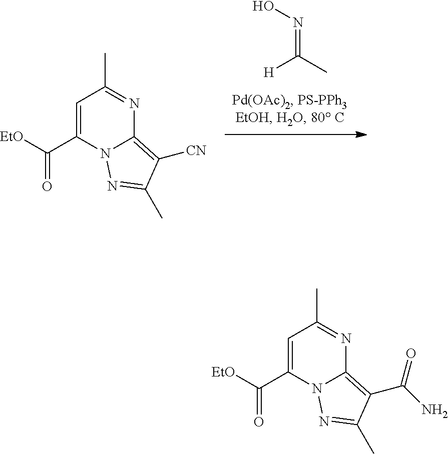

Ethyl 3-cyano-2,5-dimethyl-pyrazolo[1,5-a]pyrimidine-7-carboxylate (1.38 g, 5.65 mmol) was suspended in EtOH (10 mL) and water (2 mL) and the mixture was degassed by sparging with a stream of argon. Acetaldehyde oxime (2.75 mL, 45.20 mmol), Pd(OAc)₂ (190 mg, 0.85 mmol), and PS-PPh₃ (1.4-2 mmol/g; 1.11 g, 1.55-2.22 mmol) were added and the reaction was heated at 80° C. for 2 h. The reaction mixture was cooled to room temperature, filtered through a short pad of Dicalite®, and concentrated in vacuo. The crude product was purified by column chromatography (eluting with 0 to 10% MeOH in DCM) to provide the title compound (962 mg).

¹H NMR (400 MHz, CDCl₃) δ 8.11 (s, 1H), 7.21 (s, 1H), 5.60 (s, 1H), 4.57 (q, J=7.2 Hz, 2H), 2.83 (s, 3H), 2.71 (s, 3H), 1.47 (t, J=7.1 Hz, 3H)

### Synthesis of 3-carbamoyl-2,5-dimethyl-pyrazolo[1,5-a]pyrimidine-7-carboxylic Acid

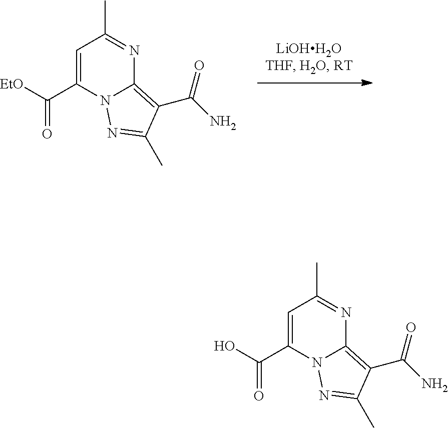 [SMILES: CCOC(=O)c1cc(C)nc2c(C(N)=O)c(C)nn12 ; Cc1cc(C(=O)O)n2nc(C)c(C(N)=O)c2n1]

A solution of LiOH·H₂O (167 mg, 4.00 mmol) in water (5 mL) was added dropwise to a solution of ethyl 3-carbamoyl-2,5-dimethyl-pyrazolo[1,5-a]pyrimidine-7-carboxylate (962 mg, 3.63 mmol) in THF (15 mL) and the reaction was stirred at room temperature for 2 h. The reaction mixture was concentrated in vacuo, diluted with water, and acidified with 5 M aq. HCl. The resulting precipitate was collected by filtration and dried to provide the title compound (826 mg).

¹H NMR (400 MHz, DMSO-d₆) δ 7.71 (s, 1H), 7.42 (s, 1H), 7.40 (s, 1H), 2.66 (s, 3H), 2.62 (s, 3H). Exchangeable proton not observed.

### Synthesis of Compound 42, N7-(3,3-difluorocyclobutyl)-2,5-dimethyl-pyrazolo[1,5-a]pyrimidine-3,7-dicarboxamide

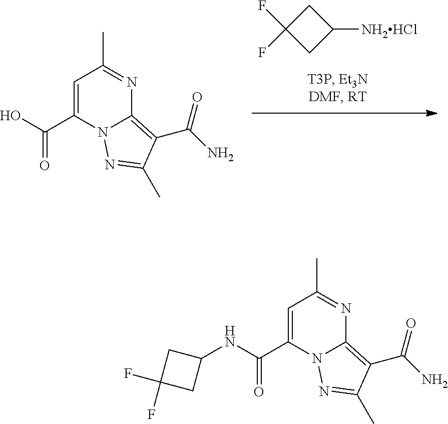

Et₃N (327 μL, 2.35 mmol), 3,3-difluorocyclobutanamine hydrochloride (135 mg, 0.94 mmol), and 1-propanephosphonic anhydride (50 wt % in DMF; 560 μL, 0.94 mmol) were added to a suspension of 3-carbamoyl-2,5-dimethyl-pyrazolo[1,5-a]pyrimidine-7-carboxylic acid (110 mg, 0.47 mmol) in DMF (1 mL) and the reaction was stirred at room temperature for 16 h. The reaction mixture was quenched with 0.5 M aq. HCl, and the resulting precipitate was collected by filtration, washed with 0.5 M aq. HCl and water, and dried. The solid was dissolved in hot DMSO, and the solution was cooled to room temperature. The resulting precipitate was collected by filtration, washed with water, and dried to provide the title compound (88 mg).

¹H NMR (400 MHz, DMSO-d₆) δ 10.16 (d, J=6.5 Hz, 1H), 7.74 (s, 1H), 7.51 (s, 1H), 7.46 (d, J=2.7 Hz, 1H), 3.16-3.01 (m, 2H), 2.89-2.71 (m, 2H), 2.69 (s, 3H), 2.67 (s, 3H). Additional exchangeable proton not observed.

LCMS (Method 2): 1.84 min, 324.2 [M+H]⁺

### Synthesis of tetrahydrofuran-2-carbonyl chloride

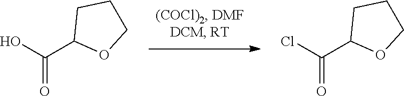 [SMILES: O=C(O)C1CCCO1 ; O=C(Cl)C1CCCO1]

DMF (121 μL, 1.57 mmol) was added to a solution of tetrahydrofuran-2-carboxylic acid (1.5 mL, 15.68 mmol) in DCM (30 mL), followed by the dropwise addition of oxalyl chloride (1.61 mL, 18.82 mmol) over 30 min. The reaction was stirred at room temperature for 1 h then the mixture was concentrated in vacuo to provide the title compound (2.11 g). The crude product was taken forward to the next step without purification.

¹H NMR (400 MHz, CDCl₃) δ 4.75 (dd, J=8.7, 5.1 Hz, 1H), 4.12-3.99 (m, 2H), 2.47-2.34 (m, 1H), 2.32-2.20 (m, 1H), 2.10-1.91 (m, 2H)

### Synthesis of 2-[hydroxy (tetrahydrofuran-2-yl)methylene]propanedinitrile

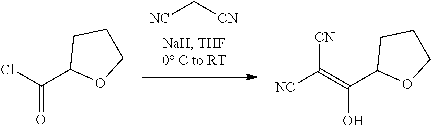 [SMILES: N#CCC#N ; O=C(Cl)C1CCCO1 ; N#CC(C#N)=C(O)C1CCCO1]

A solution of propanedinitrile (1.04 g, 15.68 mmol) in THF (15 mL) was added dropwise over 15 min to a suspension of NaH (1.25 g, 31.36 mmol) in THF (15 mL) at 0° C. The mixture was stirred at room temperature for 15 min then cooled to 0° C. A solution of tetrahydrofuran-2-carbonyl chloride (prepared as previous step, 2.11 g, 15.68 mmol) in THF (15 mL) was added dropwise over 20 min, then the reaction was warmed to room temperature and stirred for 18 h. The reaction was quenched with water, acidified with 1 M aq. HCl, and diluted with EtOAc. The layers were separated, and the aqueous layer was extracted with EtOAc. The combined organic layers were washed with brine, dried over Na₂SO₄, and concentrated in vacuo. The crude product was purified by column chromatography (eluting with 0 to 20% MeOH in DCM) to provide the title compound (1.732 g).

¹H NMR (400 MHz, MeOD) δ 4.75 (dd, J=7.9, 7.0 Hz, 1H), 4.09 (dt, J=8.0, 6.5 Hz, 1H), 3.91-3.84 (m, 1H), 2.49-2.36 (m, 1H), 2.11-1.82 (m, 4H)

LCMS (Method 2): 1.19 min, 163.2 [M−H]⁻

### Synthesis of 2-[methoxy(tetrahydrofuran-2-yl)methylene]propanedinitrile

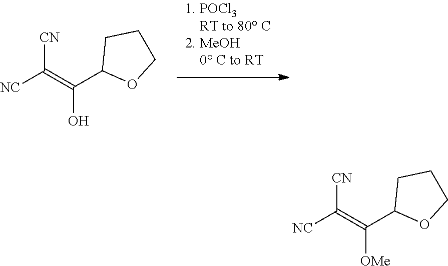 [SMILES: N#CC(C#N)=C(O)C1CCCO1 ; COC(=C(C#N)C#N)C1CCCO1]

A mixture of 2-[hydroxy (tetrahydrofuran-2-yl)methylene]propanedinitrile (1.73 g, 10.54 mmol) and POCl₃ (8.84 mL, 94.85 mmol) was stirred at room temperature for 30 min then heated at 80° C. for 1 h. The reaction mixture was cooled to room temperature and concentrated in vacuo. The residue was cooled to 0° C. and MeOH (40 mL, 900 mmol) was added dropwise. The reaction was stirred at room temperature for 30 min then concentrated in vacuo. The residue was diluted with EtOAc and water and the layers were separated. The aqueous layer was extracted with EtOAc (×2), and the combined organic layers were washed with sat. aq. NaHCO₃ and brine, dried over Na₂SO₄, and concentrated in vacuo. The crude product was purified by column chromatography (eluting with 0 to 100% EtOAc in heptane) to provide the title compound (628 mg).

¹H NMR (400 MHz, CDCl₃) δ 4.89-4.83 (m, 1H), 4.29 (s, 3H), 4.14-4.04 (m, 1H), 3.96-3.89 (m, 1H), 2.46-2.27 (m, 1H), 2.09-1.90 (m, 3H)

LCMS (Method 2): 1.71 min, 177.2 [M−H]⁻

### Synthesis of 5-amino-3-tetrahydrofuran-2-yl-1H-pyrazole-4-carbonitrile

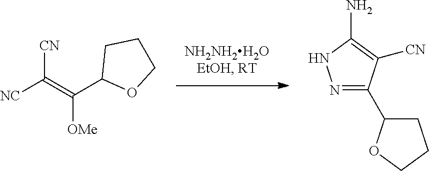 [SMILES: COC(=C(C#N)C#N)C1CCCO1 ; N#Cc1c(C2CCCO2)n[nH]c1N]

Hydrazine monohydrate (305 μL, 6.26 mmol) was added to a solution of 2-[methoxy(tetrahydrofuran-2-yl)methylene]propanedinitrile (620 mg, 3.48 mmol) in EtOH (5 mL) and the reaction was stirred at room temperature for 16 h then concentrated in vacuo. The crude product was purified by column chromatography (eluting with 0 to 10% MeOH in DCM) to provide the title compound (355 mg).

¹H NMR (400 MHz, DMSO-d₆) δ 11.78 (s, 1H), 6.25 (s, 2H), 4.69 (s, 1H), 3.91-3.79 (m, 1H), 3.75 (td, J=7.9, 5.5 Hz, 1H), 2.30-1.80 (m, 4H)

LCMS (Method 2): 1.29 min, 179.3 [M+H]⁺

### Synthesis of ethyl 3-cyano-2-tetrahydrofuran-2-yl-pyrazolo[1,5-a]pyrimidine-7-carboxylate

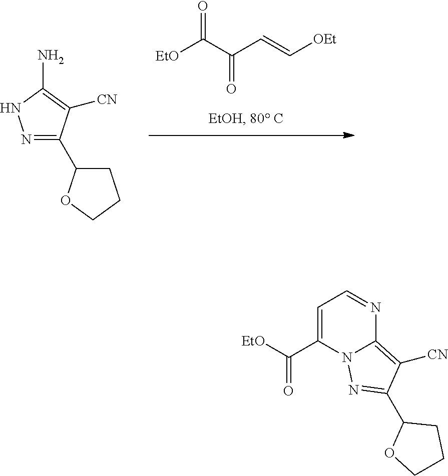 [SMILES: N#Cc1c(C2CCCO2)n[nH]c1N ; CCO/C=C/C(=O)C(=O)OCC ; CCOC(=O)c1ccnc2c(C#N)c(C3CCCO3)nn12]

A solution of 5-amino-3-tetrahydrofuran-2-yl-1H-pyrazole-4-carbonitrile (475 mg, 2.67 mmol) in EtOH (10 mL) was heated to 80° C. A solution of ethyl (E)-4-ethoxy-2-oxo-but-3-enoate (597 mg, 3.47 mmol) in EtOH (5 mL) was added, and the reaction was heated at 80° C. for 2 h. An additional portion of ethyl (E)-4-ethoxy-2-oxo-but-3-enoate (300 mg, 1.74 mmol) was added, and the reaction was heated at 80° C. for a further 2 h. The reaction mixture was cooled to room temperature and concentrated in vacuo. The crude product was purified twice by column chromatography (eluting first with 0 to 20% EtOAc in DCM and then with 0 to 50% EtOAc in heptane) to provide the title compound (430 mg).

¹H NMR (400 MHz, CDCl₃) δ 8.78 (d, J=4.2 Hz, 1H), 7.46 (d, J=4.2 Hz, 1H), 5.38-5.28 (m, 1H), 4.57 (q, J=7.1 Hz, 2H), 4.27 (dt, J=8.0, 6.6 Hz, 1H), 4.08-3.98 (m, 1H), 2.54-2.41 (m, 1H), 2.29-2.00 (m, 3H), 1.47 (t, J=7.1 Hz, 3H)

LCMS (Method 2): 2.06 min, 287.2 [M+H]⁺

### Synthesis of 3-cyano-2-tetrahydrofuran-2-yl-pyrazolo[1,5-a]pyrimidine-7-carboxylic Acid

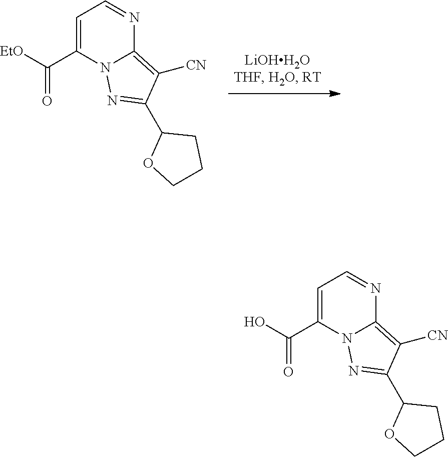 [SMILES: CCOC(=O)c1ccnc2c(C#N)c(C3CCCO3)nn12 ; N#Cc1c(C2CCCO2)nn2c(C(=O)O)ccnc12]

A solution of LiOH·H₂O (69 mg, 1.63 mmol) in water (3 mL) was added to a solution of ethyl 3-cyano-2-tetrahydrofuran-2-yl-pyrazolo[1,5-a]pyrimidine-7-carboxylate (425 mg, 1.48 mmol) in THF (3 mL) and the reaction was stirred at room temperature for 2 h then concentrated in vacuo. The residue was dissolved in DCM and water and the layers were separated. The aqueous layer was acidified to pH 2 with 1 M aq. HCl and extracted with EtOAc (×2), DCM (×2), and a 1:9 mixture of MeOH:EtOAc (×5). The combined organic layers were dried through a hydrophobic frit and concentrated in vacuo to provide the title compound (286 mg).

¹H NMR (400 MHz, DMSO-d₆) δ 8.91 (d, J=4.3 Hz, 1H), 7.62 (d, J=4.3 Hz, 1H), 5.22-5.13 (m, 1H), 4.03 (dt, J=9.8, 6.8 Hz, 1H), 3.94-3.86 (m, 1H), 2.45-2.33 (m, 1H), 2.14-1.94 (m, 3H). Exchangeable proton not observed.

LCMS (Method 2): 1.35 min, 259.2 [M+H]⁺

### Synthesis of 3-cyano-N-(3,3-difluorocyclobutyl)-2-tetrahydrofuran-2-yl-pyrazolo[1,5-a]pyrimidine-7-carboxamide

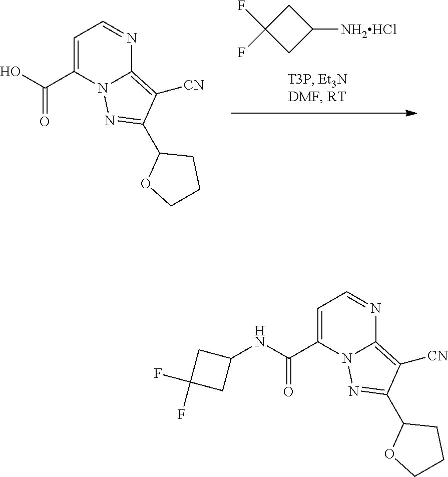

3,3-Difluorocyclobutanamine hydrochloride (156 mg, 1.08 mmol), 1-propanephosphonic anhydride (50 wt % in DMF; 790 μL, 1.36 mmol), and Et₃N (380 μL, 2.71 mmol) were added to a solution of 3-cyano-2-tetrahydrofuran-2-yl-pyrazolo[1,5-a]pyrimidine-7-carboxylic acid (140 mg, 0.54 mmol) in DMF (3 mL) and the reaction was stirred at room temperature for 18 h. The reaction was quenched with sat. aq. NaHCO₃ and diluted with EtOAc. The layers were separated, and the aqueous layer was extracted with EtOAc (×2). The combined organic layers were washed with brine, dried over Na₂SO₄, and concentrated in vacuo. The crude product was purified by column chromatography (eluting with 0 to 100% EtOAc in heptane) to provide the title compound (109 mg).

¹H NMR (400 MHz, CDCl₃) δ 10.42 (d, J=6.5 Hz, 1H), 8.92 (d, J=4.3 Hz, 1H), 7.99 (d, J=4.3 Hz, 1H), 5.41-5.30 (m, 1H), 4.61-4.47 (m, 1H), 4.32-4.23 (m, 1H), 4.15-4.05 (m, 1H), 3.28-3.13 (m, 2H), 2.80-2.64 (m, 2H), 2.63-2.49 (m, 1H), 2.34-2.07 (m, 3H)

LCMS (Method 2): 2.25 min, 348.2 [M+H]⁺

### Synthesis of Compound 43, N7-(3,3-difluorocyclobutyl)-2-tetrahydrofuran-2-yl-pyrazolo[1,5-a]pyrimidine-3,7-dicarboxamide

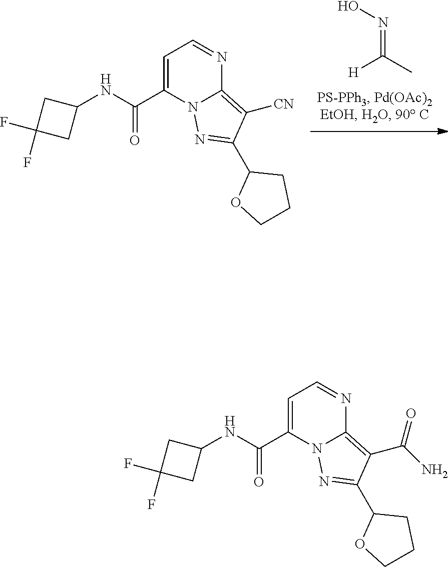

3-Cyano-N-(3,3-difluorocyclobutyl)-2-tetrahydrofuran-2-yl-pyrazolo[1,5-a]pyrimidine-7-carboxamide (105 mg, 0.30 mmol), acetaldehyde oxime (74 μL, 1.21 mmol), Pd(OAc)₂ (10 mg, 0.05 mmol), and PS-PPh₃ (1.4-2 mmol/g; 54 mg, 0.08-0.11 mmol) were suspended in EtOH (4 mL) and water (1 mL) and the reaction was heated at 90° C. for 2 h. An additional portion of Pd(OAc)₂ (10 mg, 0.05 mmol) was added and the reaction was heated at 90° C. for a further 2 h. The reaction mixture was cooled to room temperature, filtered through a short pad of Dicalite®, and concentrated in vacuo. The crude product was purified by column chromatography (eluting with 0 to 7% MeOH in DCM) to provide the title compound (44 mg).

¹H NMR (400 MHz, DMSO-d₆) δ 10.32 (d, J=6.2 Hz, 1H), 8.91 (d, J=4.4 Hz, 1H), 7.73 (s, 1H), 7.67 (d, J=4.4 Hz, 1H), 7.59 (s, 1H), 5.91 (dd, J=7.7, 4.3 Hz, 1H), 4.39 (s, 1H), 4.10-4.00 (m, 1H), 3.96-3.86 (m, 1H), 3.17 (s, 2H), 2.77-2.61 (m, 2H), 2.43-2.31 (m, 1H), 2.11-1.92 (m, 3H)

LCMS (Method 2): 1.98 min, 366.2 [M+H]⁺

### Synthesis of 3-cyano-N-indan-2-yl-2-tetrahydrofuran-2-yl-pyrazolo[1,5-a]pyrimidine-7-carboxamide

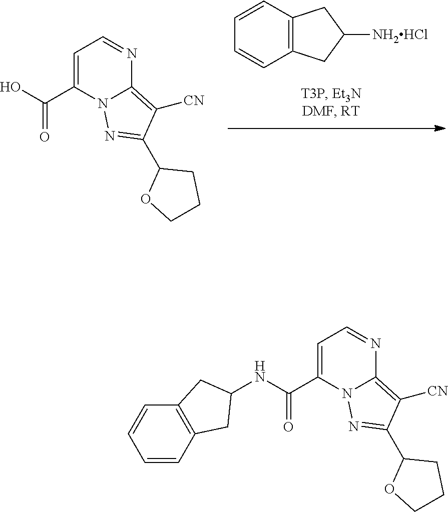

Indan-2-amine hydrochloride (138 mg, 0.81 mmol), 1-propanephosphonic anhydride (50 wt % in DMF) (630 μL, 1.08 mmol), and Et₃N (300 μL, 2.17 mmol) were added to a solution of 3-cyano-2-tetrahydrofuran-2-yl-pyrazolo[1,5-a]pyrimidine-7-carboxylic acid (140 mg, 0.54 mmol) in DMF (3 mL) and the reaction was stirred at room temperature for 18 h. The reaction was quenched with sat. aq. NaHCO₃ and diluted with EtOAc. The layers were separated, and the aqueous layer was extracted with EtOAc (×2). The combined organic layers were washed with brine, dried over Na₂SO₄, and concentrated in vacuo. The crude product was purified by column chromatography (eluting with 0 to 100% EtOAc in heptane) to provide the title compound (126 mg).

¹H NMR (400 MHz, CDCl₃) δ 10.31 (d, J=7.6 Hz, 1H), 8.87 (d, J=4.3 Hz, 1H), 7.99 (d, J=4.3 Hz, 1H), 7.32-7.26 (m, 2H), 7.24-7.20 (m, 2H), 5.19-5.13 (m, 1H), 5.06-4.95 (m, 1H), 4.07-3.91 (m, 2H), 3.47 (dd, J=16.1, 6.8 Hz, 2H), 3.02 (dd, J=16.0, 4.2 Hz, 2H), 2.38-2.23 (m, 1H), 2.05-1.93 (m, 3H)

LCMS (Method 2): 2.56 min, 374.2 [M+H]⁺

### Synthesis of Compound 44, N7-indan-2-yl-2-tetrahydrofuran-2-yl-pyrazolo[1,5-a]pyrimidine-3,7-dicarboxamide

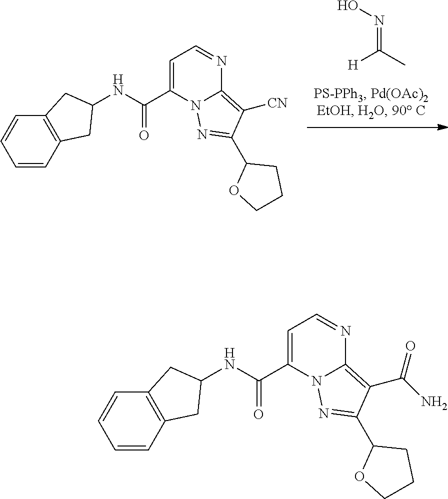

3-Cyano-N-indan-2-yl-2-tetrahydrofuran-2-yl-pyrazolo[1,5-a]pyrimidine-7-carboxamide (120 mg, 0.32 mmol), acetaldehyde oxime (78 μL, 1.29 mmol), Pd(OAc)₂ (11 mg, 0.05 mmol), and PS-PPh₃ (1.4-2 mmol/g; 57 mg, 0.08-0.11 mmol) were suspended in EtOH (4 mL) and water (1 mL) and the reaction was heated at 90° C. for 2 h. An additional portion of Pd(OAc)₂ (11 mg, 0.05 mmol) was added and the reaction was heated at 90° C. for a further 2 h. The reaction mixture was cooled to room temperature, filtered through a short pad of Dicalite®, and concentrated in vacuo. The crude product was purified by column chromatography (eluting with 0 to 7% MeOH in DCM) to provide the title compound (29 mg).

¹H NMR (400 MHz, DMSO-d₆) δ 10.28 (d, J=7.2 Hz, 1H), 8.90 (d, J=4.4 Hz, 1H), 7.78-7.70 (m, 2H), 7.56 (s, 1H), 7.34-7.27 (m, 2H), 7.22-7.16 (m, 2H), 5.87 (dd, J=7.6, 4.0 Hz, 1H), 4.86-4.78 (m, 1H), 3.79-3.67 (m, 2H), 3.45-3.36 (m, 2H), 3.00-2.92 (m, 2H), 2.29-2.19 (m, 1H), 1.89-1.79 (m, 3H)

LCMS (Method 2): 2.25 min, 392.2 [M+H]⁺

### Synthesis of ethyl 3-cyano-2-tetrahydrofuran-3-yl-pyrazolo[1,5-a]pyrimidine-7-carboxylate

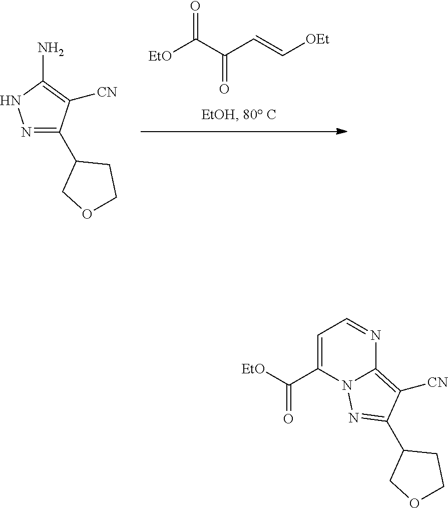 [SMILES: N#Cc1c(C2CCOC2)n[nH]c1N ; CCO/C=C/C(=O)C(=O)OCC ; CCOC(=O)c1ccnc2c(C#N)c(C3CCOC3)nn12]

Ethyl (E)-4-ethoxy-2-oxo-but-3-enoate (563 mg, 3.27 mmol) and 5-amino-3-tetrahydrofuran-3-yl-1H-pyrazole-4-carbonitrile (583 mg, 3.27 mmol) were dissolved in EtOH (20 mL) and the reaction was heated at 80° C. for 2 h. An additional portion of ethyl (E)-4-ethoxy-2-oxo-but-3-enoate (563 mg, 3.27 mmol) was added and the reaction was heated at 80° C. for a further 2 h. The reaction mixture was cooled to room temperature and concentrated in vacuo. The crude product was purified by column chromatography (eluting with 0 to 2% MeOH in DCM) to provide the title compound (366 mg).

¹H NMR (400 MHz, CDCl₃) δ 8.78 (d, J=4.2 Hz, 1H), 7.45 (d, J=4.2 Hz, 1H), 4.57 (q, J=7.1 Hz, 2H), 4.31-4.26 (m, 1H), 4.16-3.97 (m, 3H), 3.95-3.83 (m, 1H), 2.58-2.35 (m, 2H), 1.48 (t, J=7.1 Hz, 3H)

LCMS (Method 2): 2.02 min, 287.2 [M+H]⁺

### Synthesis of 3-cyano-2-tetrahydrofuran-3-yl-pyrazolo[1,5-a]pyrimidine-7-carboxylic Acid

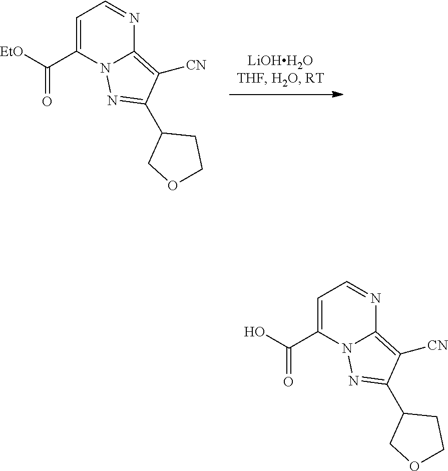 [SMILES: CCOC(=O)c1ccnc2c(C#N)c(C3CCOC3)nn12 ; N#Cc1c(C2CCOC2)nn2c(C(=O)O)ccnc12]

A solution of LiOH·H₂O (59 mg, 1.41 mmol) in water (3.2 mL) was added to a solution of ethyl 3-cyano-2-tetrahydrofuran-3-yl-pyrazolo[1,5-a]pyrimidine-7-carboxylate (366 mg, 1.28 mmol) in THF (3.2 mL) and the reaction was stirred at room temperature for 2 h then concentrated in vacuo. The residue was dissolved in EtOAc and water and the layers were separated. The aqueous layer was acidified to pH 2 with 1 M aq. HCl and extracted with EtOAc (×2) and DCM (×2). The combined organic layers were dried over Na₂SO₄ and concentrated in vacuo to provide the title compound (308 mg).

¹H NMR (400 MHz, CDCl₃) δ 8.98 (d, J=4.3 Hz, 1H), 7.97 (d, J=4.3 Hz, 1H), 4.32-4.25 (m, 1H), 4.20-4.10 (m, 1H), 4.08-3.89 (m, 3H), 2.65-2.51 (m, 1H), 2.44-2.30 (m, 1H). Exchangeable proton not observed.

LCMS (Method 2): 1.46 min, 259.2 [M+H]⁺

### Synthesis of 3-cyano-N-(3,3-difluorocyclobutyl)-2-tetrahydrofuran-3-yl-pyrazolo[1,5-a]pyrimidine-7-carboxamide

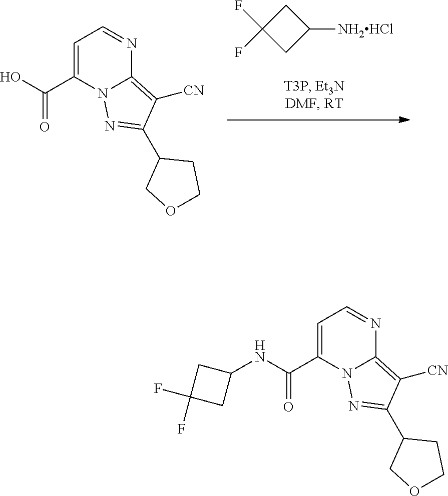

3,3-Difluorocyclobutanamine hydrochloride (257 mg, 1.79 mmol), 1-propanephosphonic anhydride (50 wt % in DMF; 840 μL, 1.43 mmol), and Et₃N (830 μL, 5.96 mmol) were added sequentially to a solution of 3-cyano-2-tetrahydrofuran-3-yl-pyrazolo[1,5-a]pyrimidine-7-carboxylic acid (308 mg, 1.19 mmol) in DMF (12 mL) and the reaction was stirred at room temperature for 3 h. Additional portions of 3,3-difluorocyclobutanamine hydrochloride (514 mg, 3.58 mmol), 1-propanephosphonic anhydride (50 wt % in DMF) (840 μL, 1.43 mmol), and Et₃N (830 μL, 5.96 mmol) were added in total over the course of a further 21 h. The reaction mixture was diluted with EtOAc and washed with sat. aq. LiCl (×3). The organic layer was dried over Na₂SO₄ and concentrated in vacuo. The crude product was purified by column chromatography (eluting with 0 to 2% MeOH in DCM) to provide the title compound (171 mg).

¹H NMR (400 MHz, CDCl₃) δ 8.89 (d, J=4.3 Hz, 1H), 7.97 (d, J=4.3 Hz, 1H), 4.56-4.45 (m, 1H), 4.31-4.21 (m, 1H), 4.19-3.99 (m, 4H), 3.99-3.87 (m, 1H), 3.26-3.10 (m, 2H), 2.73-2.53 (m, 2H), 2.40-2.28 (m, 1H). Exchangeable proton not observed.

LCMS (Method 2): 2.17 min, 346.2 [M−H]⁻

### Synthesis of Compound 45, N7-(3,3-difluorocyclobutyl)-2-tetrahydrofuran-3-yl-pyrazolo[1,5-a]pyrimidine-3,7-dicarboxamide

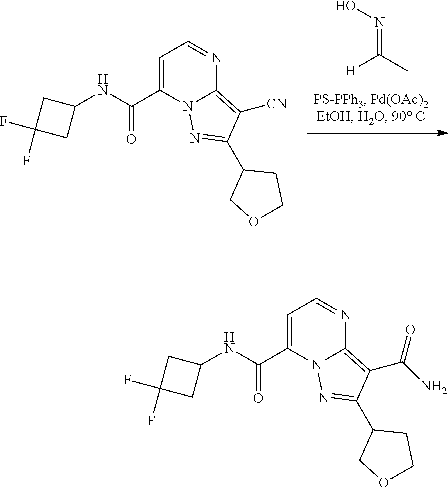

3-Cyano-N-(3,3-difluorocyclobutyl)-2-tetrahydrofuran-3-yl-pyrazolo[1,5-a]pyrimidine-7-carboxamide (91 mg, 0.26 mmol), acetaldehyde oxime (64 μL, 1.05 mmol), Pd(OAc)₂ (9 mg, 0.04 mmol), and PS-PPh₃ (1.4-2 mmol/g; 47 mg, 0.07-0.09 mmol) were suspended in EtOH (2 mL) and water (500 μL) and the mixture was degassed by sparging with a stream of argon for 5 min. The reaction was heated at 90° C. for 3 h. The reaction mixture was cooled to room temperature, filtered through a short pad of Dicalite®, and concentrated in vacuo. The crude product was purified by column chromatography (eluting with 0 to 7% MeOH in DCM) to provide the title compound (39 mg).

¹H NMR (400 MHz, DMSO-d₆) δ 10.24 (d, J=6.6 Hz, 1H), 8.91 (d, J=4.4 Hz, 1H), 7.76 (d, J=2.6 Hz, 1H), 7.66 (d, J=4.4 Hz, 1H), 7.57 (s, 1H), 4.49-4.31 (m, 2H), 4.14 (dd, J=8.2, 7.4 Hz, 1H), 3.99-3.91 (m, 1H), 3.90-3.79 (m, 2H), 3.22-3.06 (m, 2H), 2.80-2.63 (m, 2H), 2.39-2.26 (m, 2H)

LCMS (Method 2): 1.95 min, 366.2 [M+H]⁺

### Synthesis of Compound 203, 2-tetrahydrofuran-2-yl-N7-[cis-3-(trifluoromethoxy)cyclobutyl]pyrazolo[1,5-a]pyrimidine-3,7-dicarboxamide

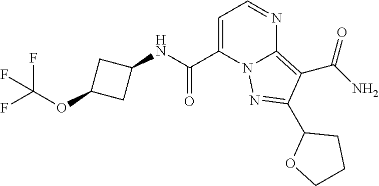 [SMILES: NC(=O)c1c(C2CCCO2)nn2c(C(=O)N[C@H]3C[C@@H](OC(F)(F)F)C3)ccnc12]

2-Tetrahydrofuran-2-yl-N7-[cis-3-(trifluoromethoxy)cyclobutyl]pyrazolo[1,5-a]pyrimidine-3,7-dicarboxamide (31 mg) was synthesised following a similar procedure to that described in General Method F.

¹H NMR (400 MHz, DMSO-d₆) δ 10.21 (d, J=7.4 Hz, 1H), 8.90 (d, J=4.4 Hz, 1H), 7.72 (s, 1H), 7.65 (d, J=4.4 Hz, 1H), 7.58 (s, 1H), 5.95-5.88 (m, 1H), 4.79-4.67 (m, 1H), 4.29-4.14 (m, 1H), 4.11-4.02 (m, 1H), 3.97-3.87 (m, 1H), 3.00-2.90 (m, 2H), 2.42-2.34 (m, 1H), 2.26 (d, J=9.9 Hz, 2H), 2.09-1.94 (m, 3H)

LCMS (Method 7): 2.50 min, 414.2 [M+H]⁺

### Synthesis of Compound 46, N7-indan-2-yl-2-methyl-pyrazolo[1,5-a]pyrimidine-3,7-dicarboxamide (General Method C)

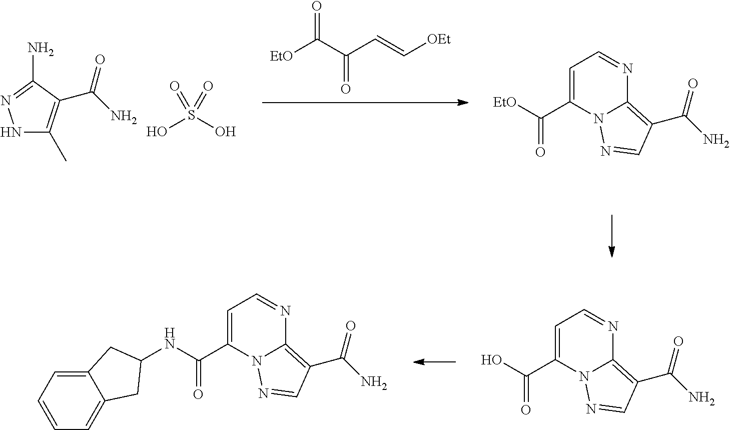

### Synthesis of ethyl 3-carbamoyl-2-methyl-pyrazolo[1,5-a]pyrimidine-7-carboxylate

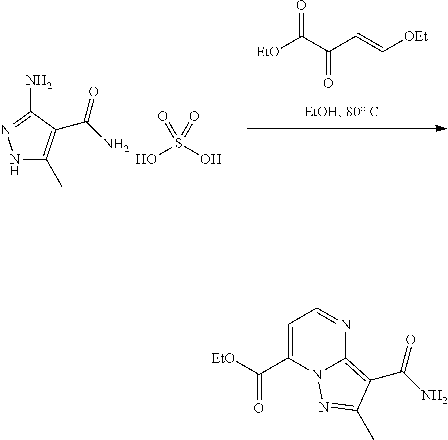

A solution of 5-amino-3-methyl-1H-pyrazole-4-carboxamide sulfuric acid salt (1 g, 4.20 mmol) and ethyl (E)-4-ethoxy-2-oxo-but-3-enoate (1.08 g, 6.30 mmol) in EtOH (10 mL) was heated at 80° C. for 3 h. The reaction mixture was cooled to room temperature and concentrated in vacuo. The crude product was purified by column chromatography (eluting with 0 to 20% MeCN in DCM) to provide the title compound (420 mg).

¹H NMR (400 MHz, DMSO-d₆) δ 8.85 (d, J=4.3 Hz, 1H), 7.63 (br. s, 1H), 7.56 (d, J=4.3 Hz, 1H), 7.49 (br. s, 1H), 4.49 (q, J=7.1 Hz, 2H), 2.66 (s, 3H), 1.37 (t, J=7.1 Hz, 3H) LCMS (Method 1): 1.34 min, 249.2 [M+H]⁺

### Synthesis of 3-carbamoyl-2-methyl-pyrazolo[1,5-a]pyrimidine-7-carboxylic Acid

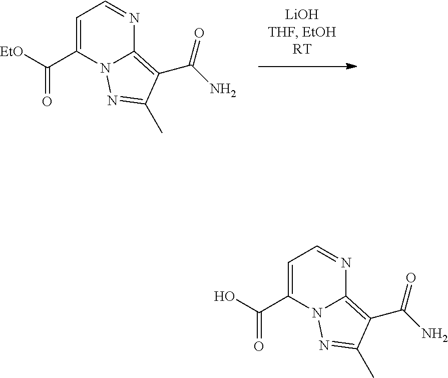 [SMILES: CCOC(=O)c1ccnc2c(C(N)=O)c(C)nn12 ; Cc1nn2c(C(=O)O)ccnc2c1C(N)=O]

A solution of LiOH·H₂O (27 mg, 14.05 mmol) in water (500 μL) was added to a solution of ethyl 3-carbamoyl-2-methyl-pyrazolo[1,5-a]pyrimidine-7-carboxylate (146 mg, 12.77 mmol) in THF (30 mL) and EtOH (500 μL) and the reaction was stirred at room temperature for 18 h. The reaction mixture was concentrated in vacuo. Water was added to the residue and the mixture was acidified with 5 M aq. HCl. The resulting precipitate was collected by filtration, washed with water, and dried to provide the title compound (129 mg).

¹H NMR (400 MHz, DMSO-d₆) δ 8.81 (d, J=4.3 Hz, 1H), 7.65 (s, 1H), 7.49-7.41 (m, 2H), 2.66 (s, 3H). Exchangeable proton not observed.

LCMS (Method 1): 1.04 min, 221.2 [M+H]⁺

### Synthesis of 46, N7-indan-2-yl-2-methyl-pyrazolo[1,5-a]pyrimidine-3,7-dicarboxamide

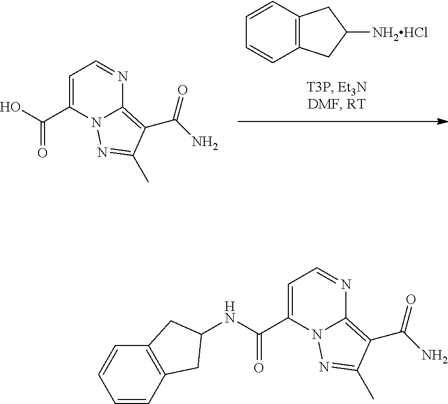

Et₃N (240 μL, 1.76 mmol) and 1-propanephosphonic anhydride (50 wt % in DMF; 1.05 mL, 1.76 mmol) were added to a solution of 3-carbamoyl-2-methyl-pyrazolo[1,5-a]pyrimidine-7-carboxylic acid (129 mg, 0.59 mmol) and indan-2-amine hydrochloride (109 mg, 0.64 mmol) in DMF (2.2 mL) and the reaction was stirred at room temperature for 64 h. The reaction mixture was diluted with water and the resulting precipitate was collected by filtration, washed with water, and dried. The crude product was purified by column chromatography (eluting with 0 to 3% MeOH in DCM) to provide the title compound (119 mg).

¹H NMR (400 MHz, DMSO-d₆) δ 10.15 (d, J=7.1 Hz, 1H), 8.86 (d, J=4.4 Hz, 1H), 7.66 (s, 1H), 7.61 (d, J=4.4 Hz, 1H), 7.49 (s, 1H), 7.29 (dd, J=5.4, 3.3 Hz, 2H), 7.24-7.14 (m, 2H), 4.83-4.75 (m, 1H), 3.40-3.35 (m, 2H), 2.99 (dd, J=16.0, 5.3 Hz, 2H), 2.61 (s, 3H)

LCMS (Method 2): 2.27 min, 336.2 [M+H]⁺

### Synthesis of Compound 47, N7-chroman-3-yl-2-methyl-pyrazolo[1,5-a]pyrimidine-3,7-dicarboxamide

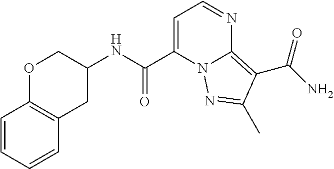 [SMILES: Cc1nn2c(C(=O)NC3COc4ccccc4C3)ccnc2c1C(N)=O]

N7-chroman-3-yl-2-methyl-pyrazolo[1,5-a]pyrimidine-3,7-dicarboxamide (34 mg) was synthesised following a similar procedure to that described in General Method C.

¹H NMR (400 MHz, DMSO-d₆) δ 10.50 (d, J=7.9 Hz, 1H), 8.88 (d, J=4.4 Hz, 1H), 7.72 (d, J=4.4 Hz, 1H), 7.65 (br. s, 1H), 7.49 (br. s, 1H), 7.19-7.13 (m, 2H), 6.93-6.87 (m, 2H), 4.66-4.60 (m, 1H), 4.29-4.21 (m, 2H), 3.26 (dd, J=16.5, 5.0 Hz, 1H), 2.87 (dd, J=16.5, 4.0 Hz, 1H), 2.47 (s, 3H)

LCMS (Method 2): 2.15 min, 352.2 [M+H]⁺

### Synthesis of Compound 48, N7-(3,3-difluorocyclobutyl)-2-methyl-pyrazolo[1,5-a]pyrimidine-3,7-dicarboxamide

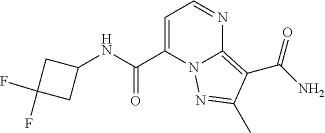 [SMILES: Cc1nn2c(C(=O)NC3CC(F)(F)C3)ccnc2c1C(N)=O]

N7-(3,3-difluorocyclobutyl)-2-methyl-pyrazolo[1,5-a]pyrimidine-3,7-dicarboxamide (20 mg) was synthesised following a similar procedure to that described in General Method C.

¹H NMR (400 MHz, DMSO-d₆) δ 10.16 (d, J=6.5 Hz, 1H), 8.88 (d, J=4.4 Hz, 1H), 7.68 (br. s, 1H), 7.59 (d, J=4.4 Hz, 1H), 7.51 (br. s, 1H), 4.42-4.30 (m, 1H), 3.15-3.03 (m, 2H), 2.88-2.75 (m, 2H), 2.71 (s, 3H)

LCMS (Method 2): 1.94 min, 310.2 [M+H]⁺

### Synthesis of Compound 49, N7-(6,7-dihydro-5H-cyclopenta[b]pyridin-6-yl)-2-methyl-pyrazolo[1,5-a]pyrimidine-3,7-dicarboxamide

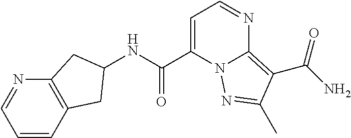 [SMILES: Cc1nn2c(C(=O)NC3Cc4cccnc4C3)ccnc2c1C(N)=O]

N7-(6,7-dihydro-5H-cyclopenta[b]pyridin-6-yl)-2-methyl-pyrazolo[1,5-a]pyrimidine-3,7-dicarboxamide (12 mg) was synthesised following a similar procedure to that described in General Method C.

¹H NMR (400 MHz, DMSO-d₆) δ 10.14 (d, J=7.1 Hz, 1H), 8.86 (d, J=4.4 Hz, 1H), 8.38-8.32 (m, 1H), 7.70-7.64 (m, 2H), 7.60 (d, J=4.4 Hz, 1H), 7.50 (s, 1H), 7.19 (dd, J=7.6, 5.0 Hz, 1H), 4.86-4.80 (m, 1H), 3.47-3.39 (m, 2H), 3.07 (t, J=5.4 Hz, 1H), 3.03 (t, J=5.2 Hz, 1H), 2.63 (s, 3H)

LCMS (Method 4): 1.27 min, 337.2 [M+H]⁺

### Synthesis of Compound 50, N7-(cis-3-cyanocyclobutyl)-2-methyl-pyrazolo[1,5-a]pyrimidine-3,7-dicarboxamide

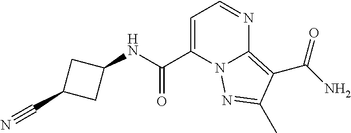 [SMILES: Cc1nn2c(C(=O)N[C@H]3C[C@@H](C#N)C3)ccnc2c1C(N)=O]

N7-(cis-3-cyanocyclobutyl)-2-methyl-pyrazolo[1,5-a]pyrimidine-3,7-dicarboxamide (21 mg) was synthesised following a similar procedure to that described in General Method C.

¹H NMR (400 MHz, DMSO-d₆) δ 10.07 (d, J=7.5 Hz, 1H), 8.87 (d, J=4.4 Hz, 1H), 7.68 (br. s, 1H), 7.58 (d, J=4.4 Hz, 1H), 7.51 (br. s, 1H), 4.56-4.44 (m, 1H), 3.25-3.11 (m, 1H), 2.80-2.73 (m, 2H), 2.72 (s, 3H), 2.55-2.49 (m, 2H). Peak at δ 2.55-2.49 overlaps with solvent peak.

LCMS (Method 2): 1.67 min, 299.2 [M+H]⁺

### Synthesis of Compound 51, N7-(5-benzylindan-2-yl)-2-methyl-pyrazolo[1,5-a]pyrimidine-3,7-dicarboxamide

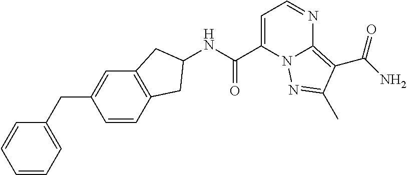 [SMILES: Cc1nn2c(C(=O)NC3Cc4ccc(Cc5ccccc5)cc4C3)ccnc2c1C(N)=O]

N7-(5-benzylindan-2-yl)-2-methyl-pyrazolo[1,5-a]pyrimidine-3,7-dicarboxamide (38 mg) was synthesised following a similar procedure to that described in General Method C.

¹H NMR (400 MHz, DMSO-d₆) δ 10.12 (d, J=7.2 Hz, 1H), 8.85 (d, J=4.4 Hz, 1H), 7.66 (s, 1H), 7.59 (d, J=4.4 Hz, 1H), 7.49 (s, 1H), 7.31-7.12 (m, 7H), 7.07 (d, J=7.9 Hz, 1H), 4.81-4.71 (m, 1H), 3.91 (s, 2H), 3.28 (s, 2H), 2.93 (dt, J=16.0, 4.5 Hz, 2H), 2.58 (s, 3H)

LCMS (Method 2): 2.68 min, 426.2 [M+H]⁺

### Synthesis of Compound 52, N7-(6,7-dihydro-5H-cyclopenta[c]pyridin-6-yl)-2-methyl-pyrazolo[1,5-a]pyrimidine-3,7-dicarboxamide

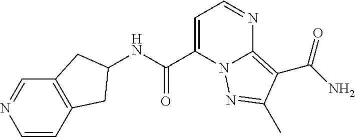 [SMILES: Cc1nn2c(C(=O)NC3Cc4ccncc4C3)ccnc2c1C(N)=O]

N7-(6,7-dihydro-5H-cyclopenta[c]pyridin-6-yl)-2-methyl-pyrazolo[1,5-a]pyrimidine-3,7-dicarboxamide (21 mg) was synthesised following a similar procedure to that described in General Method C.

¹H NMR (400 MHz, DMSO-d₆) δ 10.11 (d, J=7.1 Hz, 1H), 8.86 (d, J=4.4 Hz, 1H), 8.50 (d, J=1.0 Hz, 1H), 8.38 (d, J=4.9 Hz, 1H), 7.66 (br. s, 1H), 7.59 (d, J=4.4 Hz, 1H), 7.50 (br. s, 1H), 7.35 (dd, J=4.9, 1.0 Hz, 1H), 4.86-4.79 (m, 1H), 3.43 (dd, J=7.4, 3.3 Hz, 1H), 3.39 (dd, J=7.3, 3.9 Hz, 1H), 3.06 (d, J=5.1 Hz, 1H), 3.02 (d, J=5.0 Hz, 1H), 2.62 (s, 3H)

LCMS (Method 2): 0.91 min, 337.2 [M+H]⁺

### Synthesis of Compound 53, 2-methyl-N7-[cis-3-(trifluoromethoxy)cyclobutyl]pyrazolo[1,5-a]pyrimidine-3,7-dicarboxamide

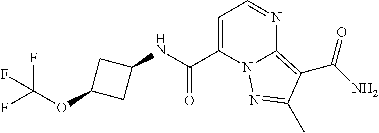 [SMILES: Cc1nn2c(C(=O)N[C@H]3C[C@@H](OC(F)(F)F)C3)ccnc2c1C(N)=O]

2-methyl-N7-[cis-3-(trifluoromethoxy)cyclobutyl]pyrazolo[1,5-a]pyrimidine-3,7-dicarboxamide (27 mg) was synthesised following a similar procedure to that described in General Method C.

¹H NMR (400 MHz, DMSO-d₆) δ 10.01 (d, J=7.3 Hz, 1H), 8.86 (d, J=4.4 Hz, 1H), 7.67 (s, 1H), 7.57 (d, J=4.4 Hz, 1H), 7.51 (s, 1H), 4.76-4.65 (m, 1H), 4.27-4.12 (m, 1H), 2.93-2.81 (m, 2H), 2.71 (s, 3H), 2.44-2.32 (m, 2H)

LCMS (Method 2): 2.20 min, 358.2 [M+H]⁺

### Synthesis of Compound 54, N8-(3,3-difluorocyclobutyl)-2-(methoxymethyl)imidazo[1,2-b]pyridazine-3,8-dicarboxamide (General Method D)

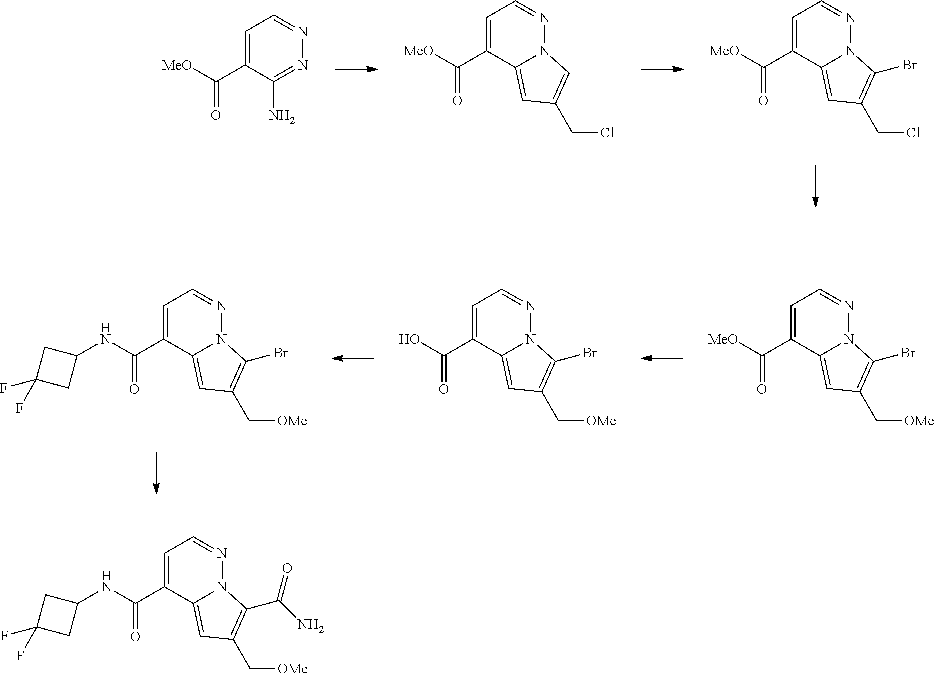

### Synthesis of methyl 2-(chloromethyl) imidazo[1,2-b]pyridazine-8-carboxylate

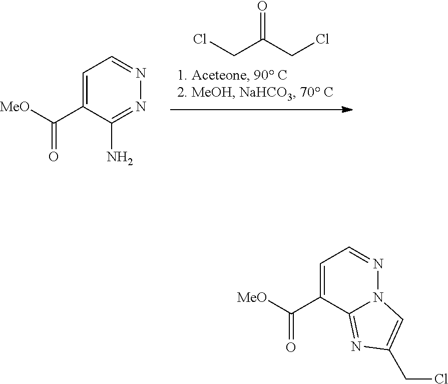 [SMILES: O=C(CCl)CCl ; COC(=O)c1ccnnc1N ; COC(=O)c1ccnn2cc(CCl)nc12]

Methyl 3-aminopyridazine-4-carboxylate (1.35 g, 8.82 mmol) and 1,3-dichloropropan-2-one (4.82 mL, 44.08 mmol) were dissolved in acetone (15 mL) and the reaction was heated at 90° C. in a microwave reactor for 4 h. The reaction mixture was cooled to room temperature and NaHCO₃ (1.48 g, 17.63 mmol) and MeOH (2 mL) were added. The reaction was heated at 70° C. for 16 h then cooled to room temperature and concentrated in vacuo. The crude product was purified by column chromatography (eluting with 0 to 20% MeOH in DCM) to provide the title compound (794 mg).

¹H NMR (400 MHz, CDCl₃) δ 8.45 (d, J=4.6 Hz, 1H), 8.14 (d, J=0.7 Hz, 1H), 7.64 (d, J=4.6 Hz, 1H), 4.87 (d, J=0.7 Hz, 2H), 4.08 (s, 3H)

LCMS (Method 1): 1.38 min, 226.2, 228.2 [M+H]⁺

### Synthesis of methyl 3-bromo-2-(chloromethyl) imidazo[1,2-b]pyridazine-8-carboxylate

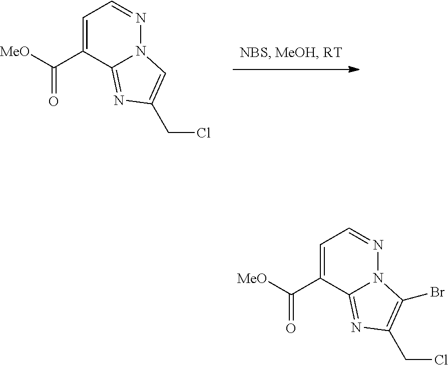 [SMILES: COC(=O)c1ccnn2cc(CCl)nc12 ; COC(=O)c1ccnn2c(Br)c(CCl)nc12]

NBS (619 mg, 3.48 mmol) was added to a solution of methyl 2-(chloromethyl) imidazo[1,2-b]pyridazine-8-carboxylate (785 mg, 3.48 mmol) in MeOH (20 mL) and the reaction was stirred at room temperature for 2 h then concentrated in vacuo. The residue was dissolved in DCM and water and the layers were separated. The aqueous layer was extracted with DCM (×2), and the combined organic layers were dried over Na₂SO₄ and concentrated in vacuo to provide the title compound (977 mg).

¹H NMR (400 MHz, CDCl₃) δ 8.60 (d, J=4.6 Hz, 1H), 7.71 (d, J=4.6 Hz, 1H), 4.89 (s, 2H), 4.09 (s, 3H)

LCMS (Method 1): 1.57 min, 304.0, 306.0 [M+H]⁺

### Synthesis of methyl 3-bromo-2-(methoxymethyl)imidazo[1,2-b]pyridazine-8-carboxylate

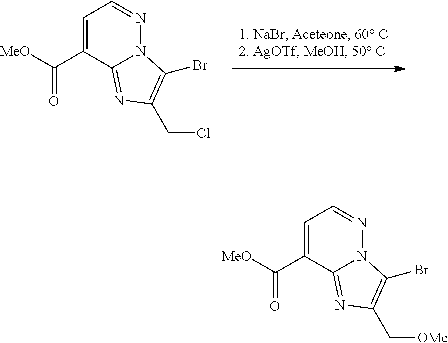 [SMILES: COC(=O)c1ccnn2c(Br)c(CCl)nc12 ; COCc1nc2c(C(=O)OC)ccnn2c1Br]

NaBr (342 mg, 3.32 mmol) was added to a solution of methyl 3-bromo-2-(chloromethyl) imidazo[1,2-b]pyridazine-8-carboxylate (920 mg, 3.02 mmol) in acetone (15 mL) and the reaction was heated at 60° C. for 5 h. The reaction mixture was cooled to room temperature and concentrated in vacuo. The residue was dissolved in DCM and water and the layers were separated. The aqueous layer was extracted with DCM (×3), and the combined organic layers were dried through a hydrophobic frit and concentrated in vacuo. The crude methyl 3-bromo-2-(bromomethyl) imidazo[1,2-b]pyridazine-8-carboxylate (850 mg, 2.44 mmol) thus obtained was dissolved in MeOH (12 mL) and AgOTf (939 mg, 3.65 mmol) was added. The reaction was heated at 50° C. for 2 h, then cooled to room temperature and concentrated in vacuo. The crude product was purified by column chromatography (eluting with 0 to 10% MeOH in DCM) to provide the title compound (1.15 g) as the impure product.

LCMS (Method 1): 1.46 min, 300.0, 302.0 [M+H]⁺

### Synthesis of 3-bromo-2-(methoxymethyl)imidazo[1,2-b]pyridazine-8-carboxylic Acid

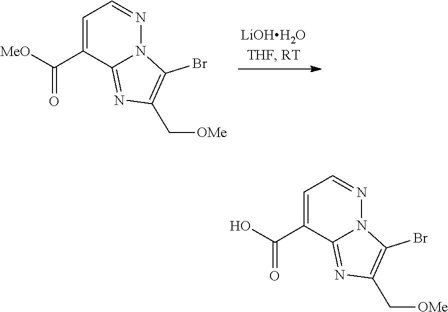 [SMILES: COCc1nc2c(C(=O)OC)ccnn2c1Br ; COCc1nc2c(C(=O)O)ccnn2c1Br]

LiOH·H₂O (112 mg, 2.68 mmol) was added to a solution of the crude methyl 3-bromo-2-(methoxymethyl)imidazo[1,2-b]pyridazine-8-carboxylate (1.15 g; generated in the previous step) in THF (15 mL) and the reaction was stirred at room temperature for 2 h then concentrated in vacuo. The residue was diluted with water and acidified to pH 2 with 1 M aq. HCl. The aqueous solution was diluted with EtOAc and the layers were separated. The aqueous layer was extracted with a 3:1 mixture of CHCl₃:IPA (×3), and the combined organic layers were dried over Na₂SO₄ and concentrated in vacuo to provide the title compound (604 mg).

¹H NMR (400 MHz, DMSO-d₆) δ 8.79 (d, J=4.6 Hz, 1H), 7.69 (d, J=4.6 Hz, 1H), 4.58 (s, 2H), 3.32 (s, 3H). Exchangeable proton not observed.

LCMS (Method 1): 1.30 min, 286.0, 288.0 [M+H]⁺

### Synthesis of 3-bromo-N-(3,3-difluorocyclobutyl)-2-(methoxymethyl)imidazo[1,2-b]pyridazine-8-carboxamide

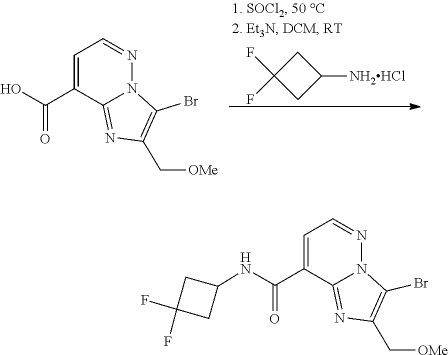

3-Bromo-2-(methoxymethyl)imidazo[1,2-b]pyridazine-8-carboxylic acid (200 mg, 0.63 mmol) was dissolved in thionyl chloride (2 mL, 0.63 mmol) and the reaction was heated at 50° C. for 1 h then cooled to room temperature. The reaction mixture was concentrated in vacuo and the residue was dissolved in DCM (5 mL). 3,3-Difluorocyclobutanamine hydrochloride (99 mg, 0.69 mmol) and Et₃N (263 μL, 1.89 mmol) were added, and the reaction was stirred at room temperature for 16 h. The reaction mixture was diluted with DCM and washed with water and sat. aq. NH₄Cl. The organic layer was dried over Na₂SO₄ and concentrated in vacuo. The crude product was purified by column chromatography (eluting with 0 to 10% MeOH in DCM) to provide the title compound (210 mg).

¹H NMR (400 MHz, CDCl₃) δ 10.01 (d, J=6.3 Hz, 1H), 8.63 (d, J=4.6 Hz, 1H), 7.92 (d, J=4.6 Hz, 1H), 4.71 (s, 2H), 4.53-4.43 (m, 1H), 3.52 (s, 3H), 3.19-3.08 (m, 2H), 2.83-2.69 (m, 2H)

LCMS (Method 1): 1.74 min, 375.0, 377.0 [M+H]⁺

### Synthesis of Compound 54, N8-(3,3-difluorocyclobutyl)-2-(methoxymethyl)imidazo[1,2-b]pyridazine-3,8-dicarboxamide

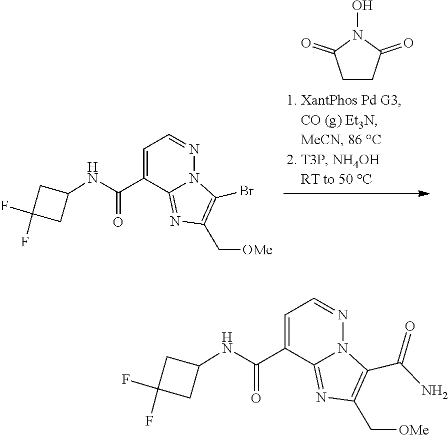 [SMILES: O=C1CCC(=O)N1O ; COCc1nc2c(C(=O)NC3CC(F)(F)C3)ccnn2c1Br ; COCc1nc2c(C(=O)NC3CC(F)(F)C3)ccnn2c1C(N)=O]

3-Bromo-N-(3,3-difluorocyclobutyl)-2-(methoxymethyl)imidazo[1,2-b]pyridazine-8-carboxamide (210 mg, 0.56 mmol), N-hydroxysuccinimide (97 mg, 0.84 mmol), XantPhos Pd G3 (53 mg, 0.06 mmol), and Et₃N (234 μL, 1.68 mmol) were dissolved in MeCN (3 mL) and the solution was degassed by sparging with a stream of argon for 30 min. The reaction vial was evacuated and backfilled with argon (×3), then evacuated and backfilled with carbon monoxide (×3). The reaction was heated at 86° C. for 20 h, then cooled to room temperature and degassed with argon. NH₄OH (33 wt % in water) (661 μL, 5.6 mmol) and 1-propanephosphonic anhydride (50 wt % in EtOAc) (1 mL, 1.68 mmol) were added, and the reaction was stirred at room temperature for 24 h then heated at 50° C. for a further 24 h. The reaction mixture was cooled to room temperature and concentrated in vacuo. The crude product was purified by column chromatography (eluting with 0 to 10% MeOH in DCM) to provide the title compound (87 mg).

¹H NMR (400 MHz, CDCl₃) δ 10.25 (d, J=6.1 Hz, 1H), 8.68 (d, J=4.7 Hz, 1H), 8.44 (s, 1H), 8.05 (d, J=4.7 Hz, 1H), 5.92 (s, 1H), 5.13 (s, 2H), 4.56-4.41 (m, 1H), 3.60 (s, 3H), 3.23-3.06 (m, 2H), 2.88-2.69 (m, 2H)

LCMS (Method 2): 1.84 min, 340.2 [M+H]⁺

### Synthesis of Compound 55, 2-(methoxymethyl)-N8-[cis-3-(trifluoromethoxy)cyclobutyl]imidazo[1,2-b]pyridazine-3,8-dicarboxamide

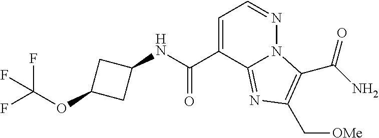 [SMILES: COCc1nc2c(C(=O)N[C@H]3C[C@@H](OC(F)(F)F)C3)ccnn2c1C(N)=O]

2-(Methoxymethyl)-N8-[cis-3-(trifluoromethoxy)cyclobutyl]imidazo[1,2-b]pyridazine-3,8-dicarboxamide (66 mg) was synthesised following a similar procedure to that described in General Method D.

¹H NMR (400 MHz, CDCl₃) δ 10.13 (d, J=7.1 Hz, 1H), 8.67 (d, J=4.7 Hz, 1H), 8.45 (s, 1H), 8.05 (d, J=4.7 Hz, 1H), 5.91 (s, 1H), 5.13 (s, 2H), 4.58-4.46 (m, 1H), 4.36-4.22 (m, 1H), 3.61 (s, 3H), 3.08-2.95 (m, 2H), 2.50-2.38 (m, 2H)

LCMS (Method 2): 2.14 min, 388.2 [M+H]⁺

### Synthesis of Compound 56, N8-indan-2-yl-2-(methoxymethyl)imidazo[1,2-b]pyridazine-3,8-dicarboxamide

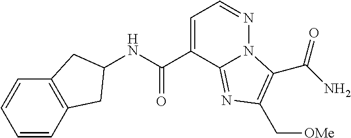 [SMILES: COCc1nc2c(C(=O)NC3Cc4ccccc4C3)ccnn2c1C(N)=O]

N8-indan-2-yl-2-(methoxymethyl)imidazo[1,2-b]pyridazine-3,8-dicarboxamide (4 mg) was synthesised following a similar procedure to that described in General Method D.

¹H NMR (400 MHz, DMSO-d₆) δ 10.07 (d, J=7.1 Hz, 1H), 8.90 (d, J=4.8 Hz, 1H), 8.17 (s, 1H), 8.04 (s, 1H), 7.93 (d, J=4.8 Hz, 1H), 7.32-7.25 (m, 2H), 7.23-7.14 (m, 2H), 4.86 (s, 2H), 4.84-4.74 (m, 1H), 3.40 (dd, J=16.2, 7.2 Hz, 2H), 3.28 (s, 3H), 2.96 (dd, J=16.1, 4.8 Hz, 2H)

LCMS (Method 2): 2.15 min, 366.2 [M+H]⁺

### Synthesis of ethyl 2-(methoxymethyl)pyrazolo[1,5-a]pyrimidine-7-carboxylate

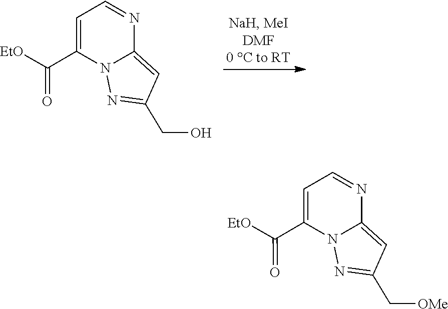 [SMILES: CCOC(=O)c1ccnc2cc(CO)nn12 ; CCOC(=O)c1ccnc2cc(COC)nn12]

NaH (83 mg, 2.08 mmol) and MeI (162 μL, 2.06 mmol) were added sequentially to a solution of ethyl 2-(hydroxymethyl)pyrazolo[1,5-a]pyrimidine-7-carboxylate (383 mg, 1.73 mmol) in DMF (6 mL) at 0° C. The reaction mixture was warmed to room temperature and stirred overnight. The reaction was quenched with water, acidified with 1 M aq. HCl, and diluted with EtOAc. The layers were separated, and the aqueous layer was extracted with EtOAc (×2). The combined organic layers were dried through a hydrophobic frit and concentrated in vacuo. The crude product was purified by column chromatography (eluting with 0 to 100% EtOAc in heptane) to provide the title compound (150 mg).

¹H NMR (400 MHz, CDCl₃) δ 8.54 (dd, J=4.1, 0.4 Hz, 1H), 7.27 (dd, J=4.1, 0.5 Hz, 1H), 6.87 (s, 1H), 4.77 (s, 2H), 4.59-4.51 (m, 2H), 3.47 (d, J=0.5 Hz, 3H), 1.48-1.43 (m, 3H)

LCMS (Method 2): 1.74 min, 236.2 [M+H]⁺

### Synthesis of ethyl 3-bromo-2-(methoxymethyl)pyrazolo[1,5-a]pyrimidine-7-carboxylate

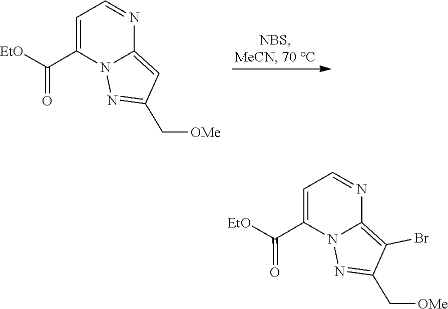 [SMILES: CCOC(=O)c1ccnc2cc(COC)nn12 ; CCOC(=O)c1ccnc2c(Br)c(COC)nn12]

NBS (136 mg, 0.77 mmol) was added to a solution of ethyl 2-(methoxymethyl)pyrazolo[1,5-a]pyrimidine-7-carboxylate (150 mg, 0.64 mmol) in MeCN (5 mL) and the reaction was heated at 70° C. for 24 h. The reaction mixture was cooled to room temperature, diluted with DCM, and washed with sat. aq. NaHCO₃(×3). The organic layer was dried through a hydrophobic frit and concentrated in vacuo. The crude product was purified by column chromatography (eluting with 0 to 100% EtOAc in heptane) to provide the title compound (186 mg).

¹H NMR (400 MHz, CDCl₃) δ 8.63 (d, J=4.1 Hz, 1H), 7.34 (d, J=4.1 Hz, 1H), 4.77 (s, 2H), 4.56 (d, J=7.1 Hz, 2H), 3.48 (s, 3H), 1.46 (t, J=7.1 Hz, 3H)

LCMS (Method 2): 2.06 min, 314.0, 316.0 [M+H]⁺

### Synthesis of 3-bromo-2-(methoxymethyl)pyrazolo[1,5-a]pyrimidine-7-carboxylic Acid

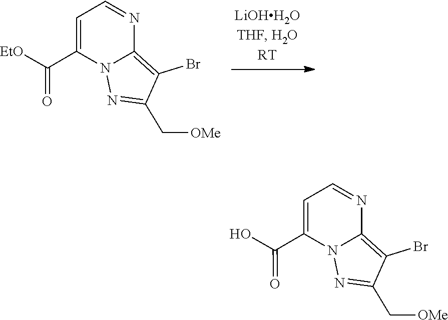 [SMILES: CCOC(=O)c1ccnc2c(Br)c(COC)nn12 ; COCc1nn2c(C(=O)O)ccnc2c1Br]

LiOH·H₂O (37 mg, 0.89 mmol) was added to a solution of ethyl 3-bromo-2-(methoxymethyl)pyrazolo[1,5-a]pyrimidine-7-carboxylate (186 mg, 0.59 mmol) in THF (1.5 mL) and water (1.5 mL) and the reaction was stirred at room temperature for 4 h. The reaction mixture was diluted with EtOAc and water and acidified to pH 2-3 with 1 M aq. HCl. The layers were separated, and the aqueous layer was extracted with EtOAc (×3). The combined organic layers were dried through a hydrophobic frit and concentrated in vacuo to provide the title compound (147 mg).

¹H NMR (400 MHz, DMSO-d₆) δ 8.73 (d, J=4.2 Hz, 1H), 7.46 (d, J=4.2 Hz, 1H), 4.61 (s, 2H), 3.33 (s, 3H). Exchangeable proton not observed.

LCMS (Method 2): 1.29 min, 286.0, 288.0 [M+H]⁺

### Synthesis of 3-bromo-N-indan-2-yl-2-(methoxymethyl)pyrazolo[1,5-a]pyrimidine-7-carboxamide

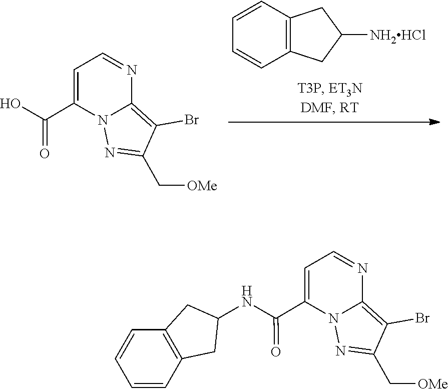

1-Propanephosphonic anhydride (50 wt % in DMF; 300 μL, 0.51 mmol) and Et₃N (360 μL, 2.57 mmol) were added sequentially to a solution of 3-bromo-2-(methoxymethyl)pyrazolo[1,5-a]pyrimidine-7-carboxylic acid (147 mg, 0.51 mmol) and indan-2-amine hydrochloride (131 mg, 0.77 mmol) in DMF (2.6 mL) and the reaction was stirred at room temperature for 18 h. The reaction mixture was diluted with EtOAc and water, and the layers were separated. The aqueous layer was extracted with EtOAc (×2), and the combined organic layers were dried through a hydrophobic frit and concentrated in vacuo. The crude product was purified by column chromatography (eluting with 0 to 100% EtOAc in heptane) to provide the title compound (95 mg).

¹H NMR (400 MHz, DMSO-d₆) δ 10.01 (d, J=7.1 Hz, 1H), 8.77 (d, J=4.2 Hz, 1H), 7.57 (d, J=4.3 Hz, 1H), 7.31-7.25 (m, 2H), 7.21-7.16 (m, 2H), 4.84-4.74 (m, 1H), 4.56 (s, 2H), 3.34 (s, 2H), 3.27 (s, 3H), 2.97 (dd, J=16.1, 5.0 Hz, 2H)

LCMS (Method 2): 2.61 min, 401.0, 403.0 [M+H]⁺

### Synthesis of Compound 57, N7-indan-2-yl-2-(methoxymethyl)pyrazolo[1,5-a]pyrimidine-3,7-dicarboxamide

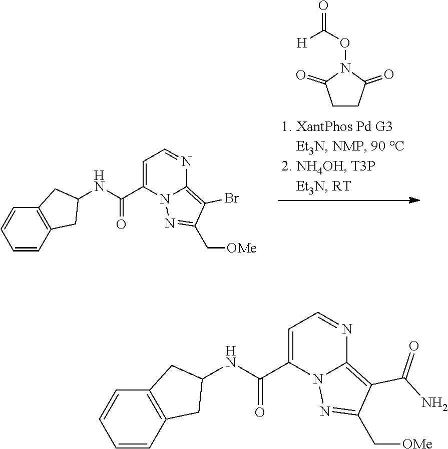 [SMILES: O=CON1C(=O)CCC1=O ; COCc1nn2c(C(=O)NC3Cc4ccccc4C3)ccnc2c1Br ; COCc1nn2c(C(=O)NC3Cc4ccccc4C3)ccnc2c1C(N)=O]

3-Bromo-N-indan-2-yl-2-(methoxymethyl)pyrazolo[1,5-a]pyrimidine-7-carboxamide (93 mg, 0.23 mmol) and XantPhos Pd G3 (22 mg, 0.02 mmol) were placed in a vial and the vial was sealed and purged with argon for 5 min. NMP (2.3 mL) and Et₃N (100 μL, 0.70 mmol) were added and the mixture was degassed by sparging with a stream of argon for 5 min. (2,5-Dioxopyrrolidin-1-yl) formate (166 mg, 1.16 mmol) was added and the vial was sealed immediately. The reaction was heated at 90° C. in a microwave reactor for 1 h. Additional portions of XantPhos Pd G3 (44 mg, 0.04 mmol), Et₃N (200 μL, 1.40 mmol), and (2,5-dioxopyrrolidin-1-yl) formate (332 mg, 2.32 mmol) were added in total and the reaction was heated at 90° C. in a microwave reactor for a further 5 h. The reaction mixture was cooled to room temperature, and Et₃N (100 μL, 0.70 mmol), 1-propanephosphonic anhydride (50 wt % in DMF; 280 μL, 0.46 mmol), and NH₄OH (240 μL, 2.32 mmol) were added. The reaction was stirred at room temperature for 18 h, then the mixture was diluted with EtOAc and water and the layers were separated. The aqueous layer was extracted with EtOAc (×2), and the combined organic layers were washed with sat. aq. LiCl, dried through a hydrophobic frit, and concentrated in vacuo. The crude product was purified by preparative HPLC (acidic method) to provide the title compound (5 mg).

¹H NMR (400 MHz, DMSO-d₆) δ 10.20 (d, J=7.0 Hz, 1H), 8.91 (d, J=4.4 Hz, 1H), 7.71-7.63 (m, 2H), 7.58 (s, 1H), 7.29 (dd, J=5.4, 3.3 Hz, 2H), 7.21-7.16 (m, 2H), 4.91 (s, 2H), 4.85-4.75 (m, 1H), 3.39 (dd, J=16.2, 7.3 Hz, 2H), 3.30 (s, 3H), 2.97 (dd, J=16.2, 4.6 Hz, 2H)

LCMS (Method 2) 2.14 min., 366.2 [M+H]⁺

### Synthesis of 3-bromo-N-(3,3-difluorocyclobutyl)-2-(methoxymethyl)pyrazolo[1,5-a]pyrimidine-7-carboxamide

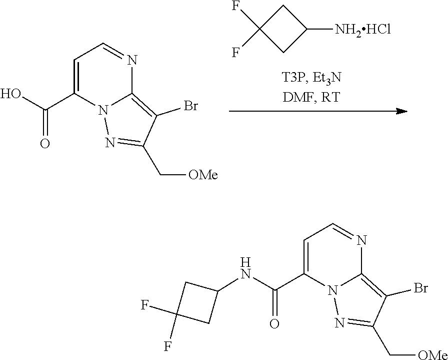

3,3-Difluorocyclobutanamine hydrochloride (298 mg, 2.08 mmol), 1-propanephosphonic anhydride (50 wt % in DMF; 890 μL, 1.52 mmol), and Et₃N (960 μL, 6.92 mmol) were added to a solution of 3-bromo-2-(methoxymethyl)pyrazolo[1,5-a]pyrimidine-7-carboxylic acid (396 mg, 1.38 mmol) in DMF (6.9 mL) and the reaction was stirred at room temperature for 72 h. Additional portions of 3,3-difluorocyclobutanamine hydrochloride (298 mg, 2.08 mmol), 1-propanephosphonic anhydride (50 wt % in DMF; 890 μL, 1.52 mmol), and Et₃N (960 UL, 6.92 mmol) were added and the reaction was stirred at room temperature for a further 4 h. The reaction mixture was diluted with EtOAc and water and the layers were separated. The aqueous layer was extracted with EtOAc (×2), and the combined organic layers were dried through a hydrophobic frit and concentrated in vacuo. The crude product was purified by column chromatography (eluting with 0 to 100% EtOAc in heptane) to provide the title compound (252 mg).

¹H NMR (400 MHz, DMSO-d₆) δ 10.00 (s, 1H), 8.79 (d, J=4.2 Hz, 1H), 7.54 (d, J=4.2 Hz, 1H), 4.66 (s, 2H), 4.35 (br. s, 1H), 3.36 (s, 3H), 3.19-3.01 (m, 2H), 2.85-2.70 (m, 2H)

### Synthesis of Compound 58, N7-(3,3-difluorocyclobutyl)-2-(methoxymethyl)pyrazolo[1,5-a]pyrimidine-3,7-dicarboxamide

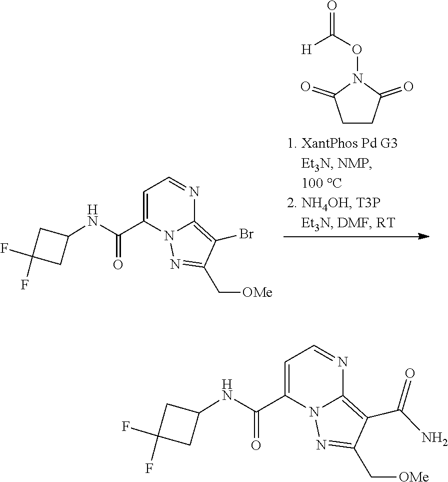 [SMILES: O=CON1C(=O)CCC1=O ; COCc1nn2c(C(=O)NC3CC(F)(F)C3)ccnc2c1Br ; COCc1nn2c(C(=O)NC3CC(F)(F)C3)ccnc2c1C(N)=O]

3-Bromo-N-(3,3-difluorocyclobutyl)-2-(methoxymethyl)pyrazolo[1,5-a]pyrimidine-7-carboxamide (180 mg, 0.48 mmol) and XantPhos Pd G3 (46 mg, 0.05 mmol) were placed in a vial and the vial was sealed and purged with argon for 5 min. NMP (4.8 mL) and Et₃N (201 μL, 1.44 mmol) were added and the mixture was degassed by sparging with a stream of argon for 5 min. (2,5-Dioxopyrrolidin-1-yl) formate (343 mg, 2.40 mmol) was added and the vial was sealed immediately. The reaction was heated at 90° C. in a microwave reactor for 2 h. The reaction was heated at 100° C. for a further 8 h, during which additional portions of XantPhos Pd G3 (69 mg, 0.07 mmol), Et₃N (305 μL, 2.20 mmol), and (2,5-dioxopyrrolidin-1-yl) formate (523 mg, 3.66 mmol) were added in total. The reaction mixture was cooled to room temperature, and Et₃N (201 μL, 1.44 mmol), 1-propanephosphonic anhydride (50 wt % in DMF; 571 μL, 0.96 mmol), and NH₄OH (243 μL, 4.80 mmol) were added. The reaction was stirred at room temperature for 18 h, then additional portions of Et₃N (201 μL, 1.44 mmol), 1-propanephosphonic anhydride (50 wt % in DMF; 571 μL, 0.96 mmol), and NH₄OH (243 μL, 4.80 mmol) were added. The reaction was stirred at room temperature for a further 72 h, then the reaction mixture was diluted with EtOAc and water. The layers were separated, and the aqueous layer was extracted with EtOAc (×3). The combined organic layers were washed with sat. aq. LiCl (×2), dried through a hydrophobic frit, and concentrated in vacuo. The crude product was purified by preparative HPLC (acidic method) to provide the title compound (31 mg).

¹H NMR (400 MHz, DMSO-d₆) δ 10.18 (d, J=6.0 Hz, 1H), 8.92 (d, J=4.3 Hz, 1H), 7.68 (s, 1H), 7.64 (d, J=4.4 Hz, 1H), 7.60 (s, 1H), 4.99 (s, 2H), 4.43-4.31 (m, 1H), 3.42 (s, 3H), 3.19-3.06 (m, 2H), 2.81-2.66 (m, 2H)

LCMS (Method 4) 1.63 min, 340.2 [M+H]⁺

### Synthesis of Compound 104, N8-(6,7-dihydro-5H-cyclopenta[b]pyridin-6-yl)-2-(methoxymethyl)imidazo[1,2-b]pyridazine-3,8-dicarboxamide

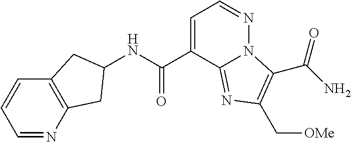 [SMILES: COCc1nc2c(C(=O)NC3Cc4cccnc4C3)ccnn2c1C(N)=O]

N8-(6,7-dihydro-5H-cyclopenta[b]pyridin-6-yl)-2-(methoxymethyl)imidazo[1,2-b]pyridazine-3,8-dicarboxamide (52 mg) was synthesised following a similar procedure to that described in General Method D.

¹H NMR (400 MHz, DMSO-d₆) δ 10.08 (d, J=7.0 Hz, 1H), 8.90 (d, J=4.8 Hz, 1H), 8.38-8.31 (m, 1H), 8.17 (s, 1H), 8.04 (s, 1H), 7.92 (d, J=4.7 Hz, 1H), 7.72-7.65 (m, 1H), 7.19 (dd, J=7.6, 4.9 Hz, 1H), 4.89-4.77 (m, 3H), 3.46 (dd, J=16.6, 7.5 Hz, 2H), 3.29 (s, 3H), 3.01 (dd, J=16.7, 4.7 Hz, 2H)

LCMS (Method 7): 1.21 min, 367.2 [M+H]⁺

### Synthesis of Compound 105, N8-(5,6-dihydro-4H-cyclopenta[c]thiophen-5-yl)-2-(methoxymethyl)imidazo[1,2-b]pyridazine-3,8-dicarboxamide

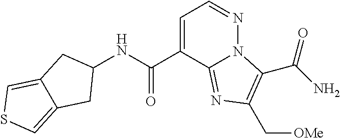 [SMILES: COCc1nc2c(C(=O)NC3Cc4cscc4C3)ccnn2c1C(N)=O]

N8-(5,6-dihydro-4H-cyclopenta[c]thiophen-5-yl)-2-(methoxymethyl)imidazo[1,2-b]pyridazine-3,8-dicarboxamide (80 mg) was synthesised following a similar procedure to that described in General Method D.

¹H NMR (400 MHz, DMSO-d₆) δ 10.11 (d, J=7.3 Hz, 1H), 8.90 (d, J=4.7 Hz, 1H), 8.18 (s, 1H), 8.05 (s, 1H), 7.92 (d, J=4.7 Hz, 1H), 7.14-7.10 (m, 2H), 5.07-4.94 (m, 1H), 4.89 (s, 2H), 3.19 (dd, J=16.0, 7.3 Hz, 2H), 2.72 (dd, J=15.8, 5.9 Hz, 2H). 3H obscured by residual H₂O peak at δ 3.35.

LCMS (Method 7): 2.24 min, 372.2 [M+H]⁺

### Synthesis of Compound 106, N8-(7-bicyclo[4.2.0]octa-1,3,5-trienyl)-2-(methoxymethyl)imidazo[1,2-b]pyridazine-3,8-dicarboxamide

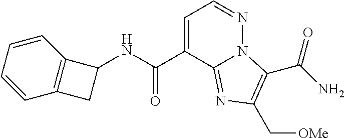 [SMILES: COCc1nc2c(C(=O)NC3Cc4ccccc43)ccnn2c1C(N)=O]

N8-(7-bicyclo[4.2.0]octa-1,3,5-trienyl)-2-(methoxymethyl)imidazo[1,2-b]pyridazine-3,8-dicarboxamide (33 mg) was synthesised following a similar procedure to that described in General Method D.

¹H NMR (400 MHz, DMSO-d₆) δ 10.32 (d, J=7.0 Hz, 1H), 8.92 (d, J=4.7 Hz, 1H), 8.19 (s, 1H), 8.06 (s, 1H), 7.96 (d, J=4.8 Hz, 1H), 7.38-7.21 (m, 4H), 5.61-5.53 (m, 1H), 4.89 (d, J=1.4 Hz, 2H), 3.78 (dd, J=14.2, 4.9 Hz, 1H), 3.30 (s, 3H), 3.16 (dd, J=14.3, 2.3 Hz, 1H)

LCMS (Method 7): 2.24 min, 352.2 [M+H]⁺

### Synthesis of Compound 107, N8-(3,3-difluoroindan-1-yl)-2-(methoxymethyl)imidazo[1,2-b]pyridazine-3,8-dicarboxamide

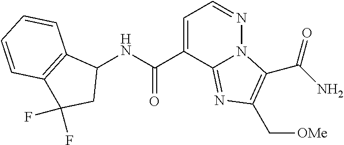 [SMILES: COCc1nc2c(C(=O)NC3CC(F)(F)c4ccccc43)ccnn2c1C(N)=O]

N8-(3,3-difluoroindan-1-yl)-2-(methoxymethyl)imidazo[1,2-b]pyridazine-3,8-dicarboxamide (81 mg) was synthesised following a similar procedure to that described in General Method D.

¹H NMR (400 MHz, DMSO-d₆) δ 10.23 (d, J=7.5 Hz, 1H), 8.93 (d, J=4.8 Hz, 1H), 8.18 (s, 1H), 8.06 (s, 1H), 7.96 (d, J=4.7 Hz, 1H), 7.71-7.53 (m, 4H), 5.77-5.65 (m, 1H), 4.93-4.80 (m, 2H), 3.30-3.15 (m, 1H), 3.27 (s, 3H), 2.66 (qd, J=14.8, 5.2 Hz, 1H)

LCMS (Method 7): 2.39 min, 402.2 [M+H]⁺

### Synthesis of Compound 108, 2-(methoxymethyl)-N8-[(4S)-7-fluorochroman-4-yl]imidazo[1,2-b]pyridazine-3,8-dicarboxamide

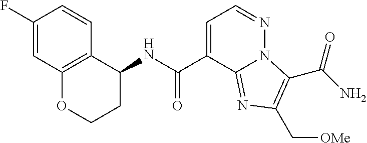 [SMILES: COCc1nc2c(C(=O)N[C@H]3CCOc4cc(F)ccc43)ccnn2c1C(N)=O]

2-(Methoxymethyl)-N8-[(4S)-7-fluorochroman-4-yl]imidazo[1,2-b]pyridazine-3,8-dicarboxamide (56 mg) was synthesised following a similar procedure to that described in General Method D.

¹H NMR (400 MHz, DMSO-d₆) δ 10.18 (d, J=7.8 Hz, 1H), 8.93 (d, J=4.8 Hz, 1H), 8.17 (s, 1H), 8.06 (s, 1H), 7.97 (d, J=4.8 Hz, 1H), 7.44-7.35 (m, 1H), 6.80-6.68 (m, 2H), 5.35-5.26 (m, 1H), 4.88 (d, J=0.9 Hz, 2H), 4.45-4.35 (m, 1H), 4.31-4.21 (m, 1H), 3.27 (s, 3H), 2.33-2.23 (m, 1H), 2.17-2.05 (m, 1H)

LCMS (Method 7): 1.74 min, 400.2 [M+H]⁺

### Synthesis of Compound 109, N8-(4,4-difluorocyclohexyl)-2-(methoxymethyl)imidazo[1,2-b]pyridazine-3,8-dicarboxamide

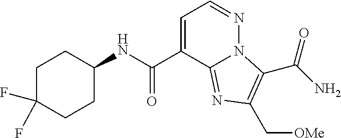

N8-(4,4-difluorocyclohexyl)-2-(methoxymethyl)imidazo[1,2-b]pyridazine-3,8-dicarboxamide (68 mg) was synthesised following a similar procedure to that described in General Method D.

¹H NMR (400 MHz, DMSO-d₆) δ 9.99 (d, J=7.6 Hz, 1H), 8.91 (d, J=4.7 Hz, 1H), 8.18 (s, 1H), 8.07 (s, 1H), 7.92 (d, J=4.7 Hz, 1H), 4.95 (s, 2H), 4.22-4.09 (m, 1H), 3.40 (s, 3H), 2.16-1.96 (m, 6H), 1.78-1.65 (m, 2H)

LCMS (Method 7): 2.24 min, 368.2 [M+H]⁺

### Synthesis of Compound 110, N8-[(4-fluorophenyl)methyl]-2-(methoxymethyl)imidazo[1,2-b]pyridazine-3,8-dicarboxamide

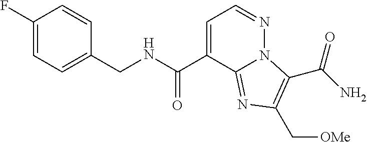 [SMILES: COCc1nc2c(C(=O)NCc3ccc(F)cc3)ccnn2c1C(N)=O]

N8-[(4-fluorophenyl)methyl]-2-(methoxymethyl)imidazo[1,2-b]pyridazine-3,8-dicarboxamide (61 mg) was synthesised following a similar procedure to that described in General Method D.

¹H NMR (400 MHz, DMSO-d₆) δ 10.09 (t, J=5.8 Hz, 1H), 8.92 (d, J=4.7 Hz, 1H), 8.20 (s, 1H), 8.06 (s, 1H), 7.94 (d, J=4.7 Hz, 1H), 7.46 (dd, J=8.4, 5.5 Hz, 2H), 7.19 (t, J=8.8 Hz, 2H), 4.92 (s, 2H), 4.69 (d, J=5.9 Hz, 2H), 3.33 (s, 3H)

LCMS (Method 7): 2.26 min, 358.2 [M+H]⁺

### Synthesis of Compound 163, 2-cyclopropyl-N8-(6,7-dihydro-5H-cyclopenta[b]pyridin-6-yl) imidazo[1,2-b]pyridazine-3,8-dicarboxamide

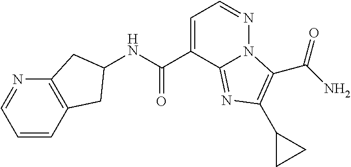 [SMILES: NC(=O)c1c(C2CC2)nc2c(C(=O)NC3Cc4cccnc4C3)ccnn12]

2-Cyclopropyl-N8-(6,7-dihydro-5H-cyclopenta[b]pyridin-6-yl) imidazo[1,2-b]pyridazine-3,8-dicarboxamide (8.3 mg) was synthesised following a similar procedure to that described in General Method D.

¹H NMR (400 MHz, DMSO-d₆) δ 10.04 (d, J=7.3 Hz, 1H), 8.82 (d, J=4.8 Hz, 1H), 8.41 (d, J=4.9 Hz, 1H), 8.19 (s, 1H), 7.96 (s, 1H), 7.87 (d, J=4.8 Hz, 1H), 7.78 (d, J=7.5 Hz, 1H), 7.27 (dd, J=7.6, 5.2 Hz, 1H), 4.85-4.79 (m, 1H), 3.49-3.43 (m, 2H), 3.14-3.08 (m, 1H), 2.98 (dt, J=16.7, 3.8 Hz, 2H), 1.03-0.96 (m, 2H), 0.79-0.71 (m, 2H)

LCMS (Method 7): 1.49 min, 363.2 [M+H]⁺

### Synthesis of Compound 164, N8-(4,6-difluoroindan-2-yl)-2-(methoxymethyl)imidazo[1,2-b]pyridazine-3,8-dicarboxamide

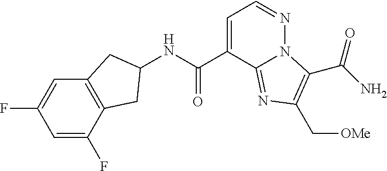 [SMILES: COCc1nc2c(C(=O)NC3Cc4cc(F)cc(F)c4C3)ccnn2c1C(N)=O]

N8-(4,6-difluoroindan-2-yl)-2-(methoxymethyl)imidazo[1,2-b]pyridazine-3,8-dicarboxamide (65 mg) was synthesised following a similar procedure to that described in General Method D.

¹H NMR (400 MHz, DMSO-d₆) δ 10.08 (d, J=7.1 Hz, 1H), 8.90 (d, J=4.8 Hz, 1H), 8.16 (s, 1H), 8.04 (s, 1H), 7.91 (d, J=4.7 Hz, 1H), 7.10-6.99 (m, 2H), 4.93-4.81 (m, 3H), 3.49-3.37 (m, 2H), 3.30 (s, 3H), 3.07-2.91 (m, 2H)

LCMS (Method 7): 2.52 min, 402.2 [M+H]⁺

### Synthesis of ethyl 3-[(4-methoxyphenyl)methylamino]-6-methyl-pyridazine-4-carboxylate

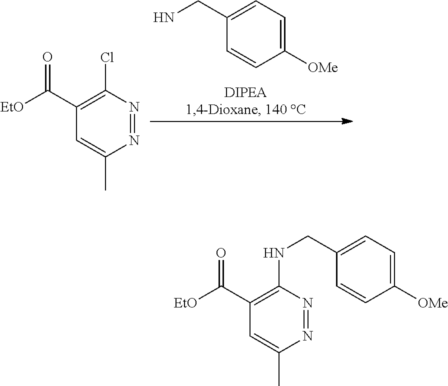 [SMILES: CCOC(=O)c1cc(C)nnc1Cl ; COc1ccc(CN)cc1 ; CCOC(=O)c1cc(C)nnc1NCc1ccc(OC)cc1]

Ethyl 3-chloro-6-methyl-pyridazine-4-carboxylate (2 g, 9.97 mmol), (4-methoxyphenyl) methanamine (2.6 mL, 19.94 mmol), and DIPEA (5.2 mL, 29.91 mmol) were dissolved in 1,4-dioxane (10 mL) and the reaction was heated at 140° C. in a microwave reactor for 2 h. The reaction mixture was concentrated in vacuo and the residue was dissolved in EtOAc and water. The layers were separated, and the organic layer was washed with brine, dried over Na₂SO₄, and concentrated in vacuo. The crude product was purified by column chromatography (eluting with 0 to 70% EtOAc in heptane) to provide the title compound (2.46 g).

¹H NMR (400 MHz, CDCl₃) δ 7.63 (s, 1H), 7.58 (s, 1H), 7.39-7.31 (m, 2H), 6.91-6.83 (m, 2H), 4.79 (d, J=5.4 Hz, 2H), 4.35 (q, J=7.1 Hz, 2H), 3.79 (s, 3H), 2.60 (s, 3H), 1.38 (t, J=7.1 Hz, 3H)

LCMS (Method 6): 2.31 mins, 302.2 [M+H]⁺

### Synthesis of ethyl 3-amino-6-methyl-pyridazine-4-carboxylate

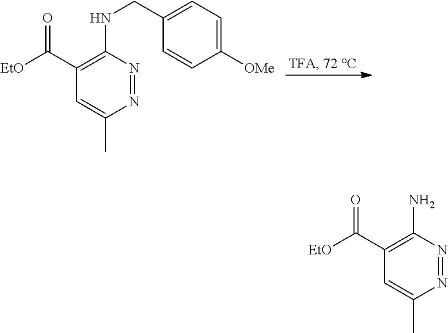 [SMILES: CCOC(=O)c1cc(C)nnc1NCc1ccc(OC)cc1 ; CCOC(=O)c1cc(C)nnc1N]

Ethyl 3-[(4-methoxyphenyl)methylamino]-6-methyl-pyridazine-4-carboxylate (3.26 g, 10.82 mmol) was suspended in TFA (24.8 mL, 324.54 mmol) and the reaction was heated at 72° C. for 2 h. The reaction mixture was cooled to room temperature, basified to pH 8 with sat. aq. NaHCO₃, and diluted with EtOAc. The layers were separated, and the organic layer was washed with water and brine, dried over Na₂SO₄, and concentrated in vacuo. The crude product was purified by column chromatography (eluting with 0 to 5% MeOH in DCM) to provide the title compound (1.48 g).

¹H NMR (400 MHz, CDCl₃) δ 7.61 (s, 1H), 6.27 (s, 2H), 4.39 (q, J=7.1 Hz, 2H), 2.62 (s, 3H), 1.41 (t, J=7.1 Hz, 3H)

LCMS (Method 6): 0.90 mins, 182.2 [M+H]⁺

### Synthesis of Compound 218, N8-(4,4-difluorocyclohexyl)-2-(methoxymethyl)-6-methyl-imidazo[1,2-b]pyridazine-3,8-dicarboxamide

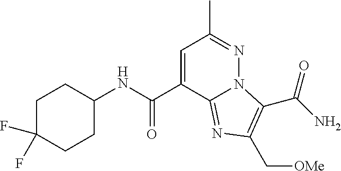 [SMILES: COCc1nc2c(C(=O)NC3CCC(F)(F)CC3)cc(C)nn2c1C(N)=O]

N8-(4,4-difluorocyclohexyl)-2-(methoxymethyl)-6-methyl-imidazo[1,2-b]pyridazine-3,8-dicarboxamide (25 mg) was synthesised following a similar procedure to that described in General Method D.

¹H NMR (400 MHz, DMSO-d₆) δ 9.98 (d, J=7.6 Hz, 1H), 8.28 (s, 1H), 8.03 (s, 1H), 7.85 (s, 1H), 4.93 (s, 2H), 4.20-4.10 (m, 1H), 3.39 (s, 3H), 2.72 (s, 3H), 2.13-1.97 (m, 6H), 1.76-1.64 (m, 2H)

LCMS (Method 7): 2.26 mins, 382.2 [M+H]⁺

### Synthesis of Compound 219, 2-(methoxymethyl)-N8-[(4R)-1,1-dioxo-3,4-dihydro-2H-thiochromen-4-yl]imidazo[1,2-b]pyridazine-3,8-dicarboxamide

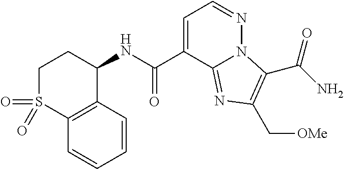 [SMILES: COCc1nc2c(C(=O)N[C@@H]3CCS(=O)(=O)c4ccccc43)ccnn2c1C(N)=O]

2-(Methoxymethyl)-N8-[(4R)-1,1-dioxo-3,4-dihydro-2H-thiochromen-4-yl]imidazo[1,2-b]pyridazine-3,8-dicarboxamide (34 mg) was synthesised following a similar procedure to that described in General Method D.

¹H NMR (400 MHz, DMSO-d₆) δ 10.22 (d, J=8.2 Hz, 1H), 8.93 (d, J=4.7 Hz, 1H), 8.18 (s, 1H), 8.07 (s, 1H), 7.94 (d, J=4.7 Hz, 1H), 7.90-7.84 (m, 1H), 7.69-7.64 (m, 2H), 7.64-7.57 (m, 1H), 5.66-5.56 (m, 1H), 4.88 (d, J=2.8 Hz, 2H), 3.91-3.80 (m, 1H), 3.73-3.62 (m, 1H), 3.28 (s, 3H), 2.70 (s, 1H), 2.64-2.53 (m, 1H)

LCMS (Method 7): 1.83 min, 430.2 [M+H]⁺

### Synthesis of Compound 220, 2-(methoxymethyl)-N8-[(4S)-1,1-dioxo-3,4-dihydro-2H-thiochromen-4-yl]imidazo[1,2-b]pyridazine-3,8-dicarboxamide

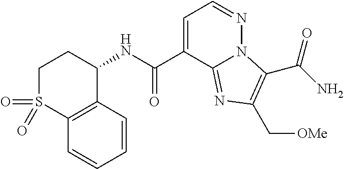 [SMILES: COCc1nc2c(C(=O)N[C@H]3CCS(=O)(=O)c4ccccc43)ccnn2c1C(N)=O]

2-(Methoxymethyl)-N8-[(4S)-1,1-dioxo-3,4-dihydro-2H-thiochromen-4-yl]imidazo[1,2-b]pyridazine-3,8-dicarboxamide (44 mg) was synthesised following a similar procedure to that described in General Method D.

¹H NMR (400 MHz, DMSO-d₆) δ10.22 (d, J=8.3 Hz, 1H), 8.93 (d, J=4.7 Hz, 1H), 8.18 (s, 1H), 8.07 (s, 1H), 7.94 (d, J=4.7 Hz, 1H), 7.90-7.84 (m, 1H), 7.70-7.57 (m, 3H), 5.69-5.56 (m, 1H), 4.88 (d, J=2.8 Hz, 2H), 3.91-3.80 (m, 1H), 3.73-3.62 (m, 1H), 3.28 (s, 3H), 2.76-2.65 (m, 1H), 2.65-2.53 (m, 1H)

LCMS (Method 7): 1.86 min, 430.1 [M+H]⁺

### Synthesis of Compound 221, N8-(1,1-dioxo-3,4-dihydro-2H-thiochromen-4-yl)-2-(methoxymethyl)imidazo[1,2-b]pyridazine-3,8-dicarboxamide

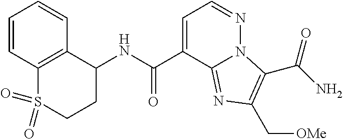 [SMILES: COCc1nc2c(C(=O)NC3CCS(=O)(=O)c4ccccc43)ccnn2c1C(N)=O]

N8-(1,1-dioxo-3,4-dihydro-2H-thiochromen-4-yl)-2-(methoxymethyl)imidazo[1,2-b]pyridazine-3,8-dicarboxamide (26 mg) was synthesised following a similar procedure to that described in General Method D.

¹H NMR (400 MHz, DMSO-d₆) δ 10.22 (d, J=8.2 Hz, 1H), 8.93 (d, J=4.7 Hz, 1H), 8.18 (s, 1H), 8.07 (s, 1H), 7.94 (d, J=4.8 Hz, 1H), 7.90-7.84 (m, 1H), 7.71-7.55 (m, 3H), 5.66-5.56 (m, 1H), 4.88 (d, J=2.9 Hz, 2H), 3.91-3.80 (m, 1H), 3.73-3.63 (m, 1H), 3.28 (s, 3H), 2.76-2.64 (m, 1H), 2.64-2.55 (m, 1H)

LCMS (Method 7): 1.82 min, 430.1 [M+H]⁺

### Synthesis of Compound 222, 2-cyclopropyl-N8-(1,1-dioxo-2,3-dihydrobenzothiophen-3-yl) imidazo[1,2-b]pyridazine-3,8-dicarboxamide

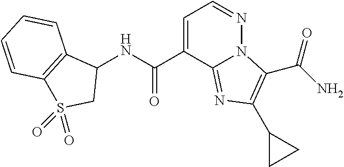 [SMILES: NC(=O)c1c(C2CC2)nc2c(C(=O)NC3CS(=O)(=O)c4ccccc43)ccnn12]

2-Cyclopropyl-N8-(1,1-dioxo-2,3-dihydrobenzothiophen-3-yl) imidazo[1,2-b]pyridazine-3,8-dicarboxamide (58 mg) was synthesised following a similar procedure to that described in General Method D.

¹H NMR (400 MHz, DMSO-d₆) δ 10.28 (d, J=7.7 Hz, 1H), δ 8.85 (d, J=4.8 Hz, 1H), 8.27-8.13 (m, 1H), 7.98 (s, 1H), 7.94-7.87 (m, 2H), 7.84-7.68 (m, 3H), 6.02-5.84 (m, 1H), 4.13 (dd, J=13.7, 7.3 Hz, 1H), 3.74 (dd, J=13.7, 4.1 Hz, 1H), 3.17-3.06 (m, 1H), 1.01-0.89 (m, 3H), 0.88-0.79 (m, 1H)

LCMS (Method 7): 2.16 min, 412.1 [M+H]⁺

### Synthesis of Compound 223, N8-(1,1-dioxo-2,3-dihydrobenzothiophen-3-yl)-2-(methoxymethyl)imidazo[1,2-b]pyridazine-3,8-dicarboxamide

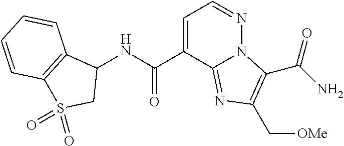 [SMILES: COCc1nc2c(C(=O)NC3CS(=O)(=O)c4ccccc43)ccnn2c1C(N)=O]

N8-(1,1-dioxo-2,3-dihydrobenzothiophen-3-yl)-2-(methoxymethyl)imidazo[1,2-b]pyridazine-3,8-dicarboxamide (99 mg) was synthesised following a similar procedure to that described in General Method D.

¹H NMR (400 MHz, DMSO-d₆) δ 10.34 (d, J=7.5 Hz, 1H), 8.94 (d, J=4.7 Hz, 1H), 8.17 (s, 1H), 8.07 (s, 1H), 7.96 (d, J=4.8 Hz, 1H), 7.90-7.87 (m, 1H), 7.82-7.74 (m, 2H), 7.73-7.67 (m, 1H), 6.02-5.92 (m, 1H), 4.90 (d, J=13.1 Hz, 1H), 4.83 (d, J=13.1 Hz, 1H), 4.17 (dd, J=13.6, 7.5 Hz, 1H), 3.78 (dd, J=13.6, 5.5 Hz, 1H), 3.26 (s, 3H)

LCMS (Method 7): 1.82 min, 416.1 [M+H]⁺

### Synthesis of Compound 59, 2-(methoxymethyl)-N7-[5-(trifluoromethyl)-2-pyridyl]pyrazolo[1,5-a]pyrimidine-3,7-dicarboxamide (General Method E)

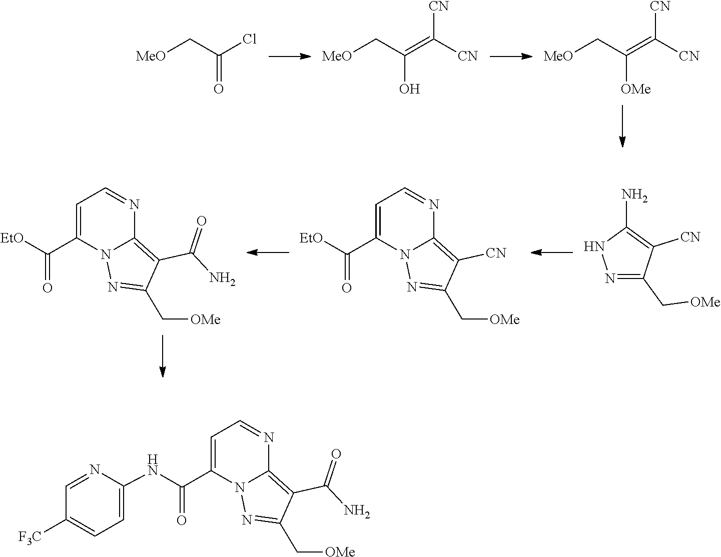

### Synthesis of 2-(1-hydroxy-2-methoxy-ethylidene)propanedinitrile

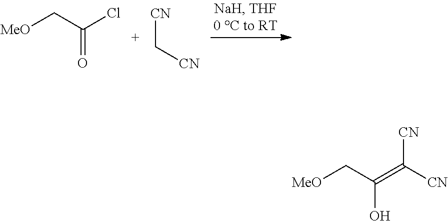

A solution of propanedinitrile (12.3 g, 186.19 mmol) in THF (100 mL) was added dropwise over 1 h to a suspension of NaH (14.9 g, 372.50 mmol) in THF (200 mL) at 0° C. and the reaction was stirred at 0° C. for 1 h. A solution of 2-methoxyacetyl chloride (17 mL, 185.95 mmol) in THF (100 mL) was added dropwise over 1 h and the reaction was warmed to room temperature and stirred for 3 h. The reaction was quenched with 5 M aq. HCl and diluted with EtOAc and water. The layers were separated, and the aqueous layer was extracted with EtOAc (×3). The combined organic layers were washed with brine, dried over Na₂SO₄, and concentrated in vacuo. The residue was dissolved in DCM and the solution was dried through a hydrophobic frit and concentrated in vacuo to provide the title compound (30.2 g). The crude product was taken forward to the next step without purification.

¹H NMR (400 MHz, CDCl₃) δ 4.45 (s, 1H), 4.08 (s, 1H), 3.60 (d, J=1.0 Hz, 3H). Exchangeable proton of enol tautomer not observed.

### Synthesis of 2-(1,2-dimethoxyethylidene)propanedinitrile

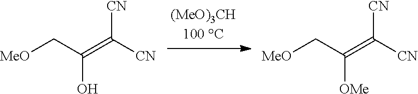 [SMILES: COCC(O)=C(C#N)C#N ; COCC(OC)=C(C#N)C#N]

The crude 2-(1-hydroxy-2-methoxy-ethylidene)propanedinitrile (30.2 g, 218.63 mmol) was dissolved in trimethoxymethane (144 mL, 1.32 mol) and the reaction was heated at 100° C. for 2 h. The reaction mixture was cooled to room temperature and concentrated in vacuo. The crude product was purified by column chromatography (eluting with 0 to 100% EtOAc in heptane) to provide the title compound (19.44 g).

¹H NMR (400 MHz, CDCl₃) δ 4.40 (s, 2H), 4.22 (s, 3H), 3.46 (s, 3H)

### Synthesis of 5-amino-3-(methoxymethyl)-1H-pyrazole-4-carbonitrile

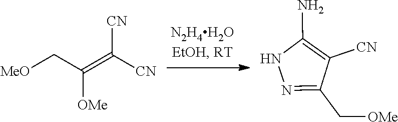 [SMILES: COCC(OC)=C(C#N)C#N ; COCc1n[nH]c(N)c1C#N]

Hydrazine monohydrate (4.9 mL, 100.82 mmol) was added to a solution of 2-(1,2-dimethoxyethylidene)propanedinitrile (15.3 g, 100.36 mmol) in EtOH (220 mL) and the reaction was stirred at room temperature for 16 h. The reaction mixture was concentrated in vacuo and the residue was azeotroped with EtOAc (×2). The crude product was recrystallised from hot EtOAc, and the resulting precipitate was collected by filtration and dried to provide the title compound (5.89 g).

¹H NMR (400 MHz, DMSO-d₆) δ 11.91 (s, 1H), 6.21 (s, 2H), 4.22 (s, 2H), 3.22 (s, 3H)

LCMS (Method 1): 0.70 min, 153.2 [M+H]⁺

### Synthesis of ethyl 3-cyano-2-(methoxymethyl)pyrazolo[1,5-a]pyrimidine-7-carboxylate

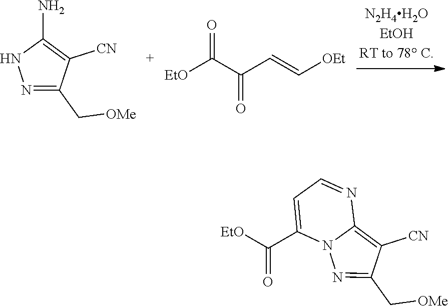 [SMILES: COCc1n[nH]c(N)c1C#N ; CCO/C=C/C(=O)C(=O)OCC ; CCOC(=O)c1ccnc2c(C#N)c(COC)nn12]

5-amino-3-(methoxymethyl)-1H-pyrazole-4-carbonitrile (5 g, 32.9 mmol) was added to a solution of ethyl (E)-4-ethoxy-2-oxo-but-3-enoate (6.8 g, 39.50 mmol) in EtOH (70 mL) and the reaction was stirred briefly at room temperature then heated at 78° C. for 16 h. The reaction mixture was concentrated in vacuo and the residue was sonicated in Et₂O for 30 min. The supernatant was decanted and the process was repeated a further three times. A suspension of the product in Et₂O was cooled to 5° C. overnight, and the resulting precipitate was collected by filtration and dried to provide the title compound (4.3 g).

¹H NMR (400 MHz, CDCl₃) δ 8.81 (d, J=4.2 Hz, 1H), 7.50 (d, J=4.2 Hz, 1H), 4.83 (s, 2H), 4.58 (q, J=7.2 Hz, 2H), 3.54 (s, 3H), 1.47 (t, J=7.1 Hz, 3H)

LCMS (Method 1): 1.53 min, 261.2 [M+H]⁺

### Synthesis of 3-carbamoyl-2-(methoxymethyl)pyrazolo[1,5-a]pyrimidine-7-carboxylic Acid

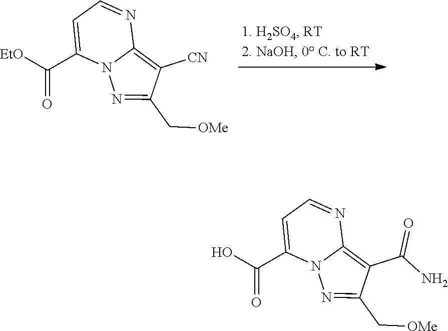 [SMILES: CCOC(=O)c1ccnc2c(C#N)c(COC)nn12 ; COCc1nn2c(C(=O)O)ccnc2c1C(N)=O]

Ethyl 3-cyano-2-(methoxymethyl)pyrazolo[1,5-a]pyrimidine-7-carboxylate (878 mg, 3.37 mmol) was dissolved in conc. H₂SO₄ (1.9 mL, 35.45 mmol) and the reaction was stirred at room temperature for 3 h. The reaction mixture was carefully added to ice-cooled 2 M aq. NaOH (53 mL, 106.00 mmol) at 0° C. and the reaction was stirred at 0° C. to room temperature over 16 h. The reaction mixture was cooled back to 0° C. and acidified with 5 M aq. HCl. The resulting precipitate was collected by filtration, washed with Et₂O, and dried to provide the title compound (730 mg).

¹H NMR (400 MHz, DMSO-d₆) δ 8.86 (d, J=4.3 Hz, 1H), 7.65 (s, 1H), 7.53 (d, J=4.3 Hz, 2H), 4.93 (s, 2H), 3.36 (s, 3H). Additional exchangeable proton not observed.

LCMS (Method 1): 1.04 min, 251.2 [M+H]⁺

### Synthesis of Compound 2-(methoxymethyl)-N7-[5-(trifluoromethyl)-2-59, pyridyl]pyrazolo[1,5-a]pyrimidine-3,7-dicarboxamide

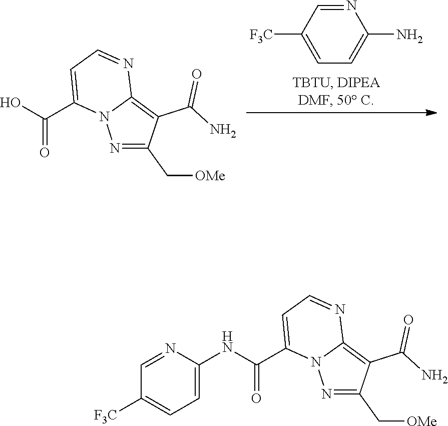 [SMILES: COCc1nn2c(C(=O)O)ccnc2c1C(N)=O ; Nc1ccc(C(F)(F)F)cn1 ; COCc1nn2c(C(=O)Nc3ccc(C(F)(F)F)cn3)ccnc2c1C(N)=O]

TBTU (90 mg, 0.28 mmol) was added to a solution of 3-carbamoyl-2-(methoxymethyl)pyrazolo[1,5-a]pyrimidine-7-carboxylic acid (50 mg, 0.20 mmol), 5-(trifluoromethyl)pyridin-2-amine (60 mL, 0.40 mmol), and DIPEA (70 mL, 0.40 mmol) in DMF (285 μL) and the reaction was heated at 50° C. for 16 h. The reaction mixture was cooled to room temperature and diluted with water. The resulting precipitate was collected by filtration, washed with 0.5 M aq. HCl, water, and Et₂O, and dried to provide the title compound (38 mg).

¹H NMR (400 MHz, DMSO-d₆) δ 12.67 (s, 1H), 8.99 (d, J=4.4 Hz, 1H), 8.87 (s, 1H), 8.49 (d, J=8.8 Hz, 1H), 8.37 (d, J=9.2 Hz, 1H), 7.87 (d, J=4.4 Hz, 1H), 7.72 (s, 1H), 7.65 (s, 1H), 5.03 (s, 2H), 3.45 (s, 3H)

LCMS (Method 2): 2.32 min, 395.2 [M+H]⁺

### Synthesis of Compound 60, N7-(6,7-dihydro-5H-cyclopenta[b]pyridin-6-yl)-2-(methoxymethyl)pyrazolo[1,5-a]pyrimidine-3,7-dicarboxamide

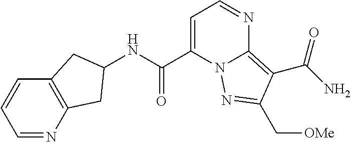 [SMILES: COCc1nn2c(C(=O)NC3Cc4cccnc4C3)ccnc2c1C(N)=O]

N7-(6,7-dihydro-5H-cyclopenta[b]pyridin-6-yl)-2-(methoxymethyl)pyrazolo[1,5-a]pyrimidine-3,7-dicarboxamide (13 mg) was synthesised following a similar procedure to that described in General Method E.

¹H NMR (400 MHz, DMSO-d₆) δ 10.21 (d, J=7.0 Hz, 1H), 8.91 (d, J=4.4 Hz, 1H), 8.35 (dd, J=5.0, 1.5 Hz, 1H), 7.72-7.67 (m, 2H), 7.66 (s, 1H), 7.58 (s, 1H), 7.19 (dd, J=7.6, 5.0 Hz, 1H), 4.92 (s, 2H), 4.90-4.78 (m, 1H), 3.51-3.40 (m, 2H), 3.31 (s, 3H), 3.06-2.96 (m, 2H)

LCMS (Method 2): 1.32 min, 367.2 [M+H]⁺

### Synthesis of Compounds 146 and 147, N7-(6,7-dihydro-5H-cyclopenta[b]pyridin-6-yl)-2-(methoxymethyl)pyrazolo[1,5-a]pyrimidine-3,7-dicarboxamide

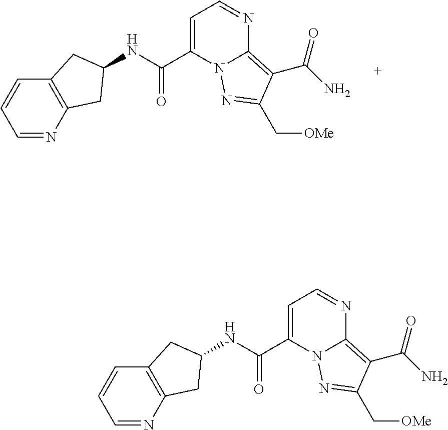 [SMILES: COCc1nn2c(C(=O)N[C@@H]3Cc4cccnc4C3)ccnc2c1C(N)=O ; COCc1nn2c(C(=O)N[C@H]3Cc4cccnc4C3)ccnc2c1C(N)=O]

Racemic N7-(6,7-dihydro-5H-cyclopenta[b]pyridin-6-yl)-2-(methoxymethyl)pyrazolo[1,5-a]pyrimidine-3,7-dicarboxamide (67 mg) was separated using preparative SFC (eluting with 30% MeOH in CO₂) to provide enantiomer 1 (25 mg) and enantiomer 2 (26 mg) with unknown absolute configurations.

¹H NMR (400 MHz, DMSO-d₆) δ 10.21 (d, J=7.0 Hz, 1H), 8.91 (d, J=4.4 Hz, 1H), 8.35 (dd, J=5.0, 1.5 Hz, 1H), 7.72-7.64 (m, 3H), 7.58 (s, 1H), 7.22-7.15 (m, 1H), 4.92 (s, 2H), 4.88-4.79 (m, 1H), 3.50-3.45 (m, 1H), 3.45-3.40 (m, 1H), 3.31 (s, 3H), 3.03 (d, J=4.5 Hz, 1H), 2.99 (d, J=4.5 Hz, 1H)

LCMS (Method 7): 1.38 min, 367.2 [M+H]⁺

### Synthesis of Compound 61, 2-(methoxymethyl)-N7-[cis-3-(trifluoromethoxy)cyclobutyl]pyrazolo[1,5-a]pyrimidine-3,7-dicarboxamide

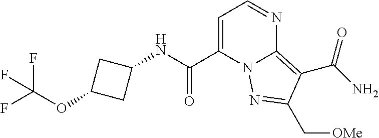 [SMILES: COCc1nn2c(C(=O)N[C@H]3C[C@@H](OC(F)(F)F)C3)ccnc2c1C(N)=O]

2-(Methoxymethyl)-N7-[cis-3-(trifluoromethoxy)cyclobutyl]pyrazolo[1,5-a]pyrimidine-3,7-dicarboxamide (22 mg) was synthesised following a similar procedure to that described in General Method E.

¹H NMR (400 MHz, DMSO-d₆) δ 10.05 (d, J=7.2 Hz, 1H), 8.91 (d, J=4.3 Hz, 1H), 7.68 (s, 1H), 7.62 (d, J=4.3 Hz, 1H), 7.59 (s, 1H), 4.99 (s, 2H), 4.75-4.68 (m, 1H), 4.25-4.16 (m, 1H), 3.43 (s, 3H), 2.95-2.87 (m, 2H), 2.35-2.27 (m, 2H)

LCMS (Method 2): 2.13 min, 388.2 [M+H]⁺

### Synthesis of Compound 62, N7-[4-[(dimethylamino)methyl]phenyl]-2-(methoxymethyl)pyrazolo[1,5-a]pyrimidine-3,7-dicarboxamide

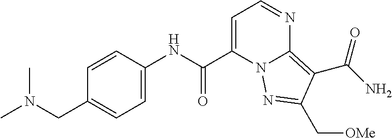 [SMILES: COCc1nn2c(C(=O)Nc3ccc(CN(C)C)cc3)ccnc2c1C(N)=O]

N7-[4-[(dimethylamino)methyl]phenyl]-2-(methoxymethyl)pyrazolo[1,5-a]pyrimidine-3,7-dicarboxamide (73 mg) was synthesised following a similar procedure to that described in General Method E.

¹H NMR (400 MHz, DMSO-d₆) δ 11.85 (s, 1H), 8.97 (d, J=4.4 Hz, 1H), 7.75 (d, J=4.4 Hz, 1H), 7.73-7.66 (m, 3H), 7.62 (s, 1H), 7.40-7.33 (m, 2H), 5.02 (s, 2H), 3.44 (s, 3H), 3.41 (s, 2H), 2.16 (s, 6H)

LCMS (Method 2): 1.31 min, 383.2 [M+H]⁺

### Synthesis of Compound 63, 2-(methoxymethyl)-N7-[(1-methylpyrazol-4-yl)methyl]pyrazolo[1,5-a]pyrimidine-3,7-dicarboxamide

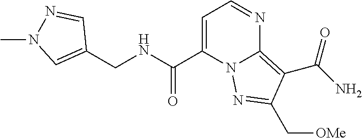 [SMILES: COCc1nn2c(C(=O)NCc3cnn(C)c3)ccnc2c1C(N)=O]

2-(methoxymethyl)-N7-[(1-methylpyrazol-4-yl)methyl]pyrazolo[1,5-a]pyrimidine-3,7-dicarboxamide (11 mg) was synthesised following a similar procedure to that described in General Method E.

¹H NMR (400 MHz, DMSO-d₆) δ 10.12 (t, J=5.4 Hz, 1H), 8.92 (d, J=4.4 Hz, 1H), 7.76-7.66 (m, 3H), 7.60 (s, 1H), 7.47 (d, J=0.8 Hz, 1H), 4.98 (s, 2H), 4.49 (d, J=5.4 Hz, 2H), 3.81 (s, 3H), 3.37 (s, 3H)

LCMS (Method 2): 1.55 min. 344.2 [M+H]⁺

### Synthesis of Compound 64, N7-(4,6-dimethyl-2-pyridyl)-2-(methoxymethyl)pyrazolo[1,5-α]pyrimidine-3,7-dicarboxamide

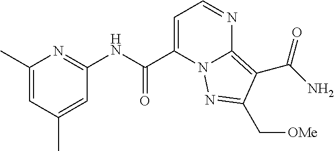 [SMILES: COCc1nn2c(C(=O)Nc3cc(C)cc(C)n3)ccnc2c1C(N)=O]

N7-(4,6-dimethyl-2-pyridyl)-2-(methoxymethyl)pyrazolo[1,5-a]pyrimidine-3,7-dicarboxamide (85 mg) was synthesised following a similar procedure to that described in General Method E.

¹H NMR (400 MHz, DMSO-d₆) δ 12.25 (s, 1H), 8.97 (d, J=4.4 Hz, 1H), 7.95 (s, 1H), 7.81 (d, J=4.4 Hz, 1H), 7.71 (s, 1H), 7.63 (s, 1H), 6.98 (s, 1H), 5.03 (s, 2H), 3.50 (s, 3H), 2.40 (s, 3H), 2.35 (s, 3H)

LCMS (Method 2): 2.12 min, 355.2 [M+H]⁺

### Synthesis of Compound 65, 2-(methoxymethyl)-N7-94-(trifluoromethyl)-2-pyridyl]pyrazolo[1,5-a]pyrimidine-3,7-dicarboxamide

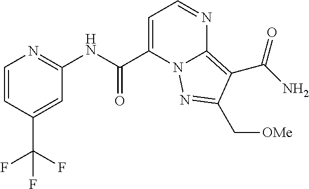 [SMILES: COCc1nn2c(C(=O)Nc3cc(C(F)(F)F)ccn3)ccnc2c1C(N)=O]

2-(Methoxymethyl)-N7-[4-(trifluoromethyl)-2-pyridyl]pyrazolo[1,5-a]pyrimidine-3,7-dicarboxamide (50 mg) was synthesised following a similar procedure to that described in General Method E.

¹H NMR (400 MHz, DMSO-d₆) δ 12.70 (s, 1H), 9.00 (d, J=4.4 Hz, 1H), 8.75 (d, J=5.1 Hz, 1H), 8.58 (s, 1H), 7.87 (d, J=4.4 Hz, 1H), 7.72 (s, 1H), 7.67 (dd, J=8.7, 4.1 Hz, 2H), 5.03 (s, 2H), 3.45 (s, 3H)

LCMS (Method 2): 2.32 min, 395.2 [M+H]⁺

### Synthesis of Compound 66, N7-(3,4-difluorophenyl)-2-(methoxymethyl)pyrazolo[1,5-α]pyrimidine-3,7-dicarboxamide

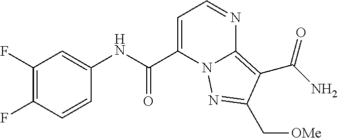 [SMILES: COCc1nn2c(C(=O)Nc3ccc(F)c(F)c3)ccnc2c1C(N)=O]

N7-(3,4-difluorophenyl)-2-(methoxymethyl)pyrazolo[1,5-a]pyrimidine-3,7-dicarboxamide (22 mg) was synthesised following a similar procedure to that described in General Method E.

¹H NMR (400 MHz, DMSO-d₆) δ 11.93 (s, 1H), 8.97 (d, J=4.4 Hz, 1H), 7.94 (ddd, J=12.6, 7.3, 2.5 Hz, 1H), 7.73 (d, J=4.4 Hz, 1H), 7.69 (s, 1H), 7.62 (s, 1H), 7.55 (dt, J=10.5, 9.0 Hz, 1H), 7.48-7.40 (m, 1H), 5.00 (s, 2H), 3.42 (s, 3H)

LCMS (Method 2): 2.27 min, 362.2 [M+H]⁺

### Synthesis of Compound 67, N7-(2,3-dihydrobenzofuran-5-ylmethyl)-2-(methoxymethyl)pyrazolo[1,5-a]pyrimidine-3,7-dicarboxamide

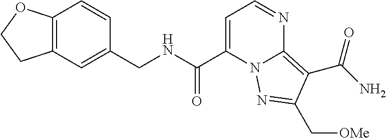 [SMILES: COCc1nn2c(C(=O)NCc3ccc4c(c3)CCO4)ccnc2c1C(N)=O]

N7-(2,3-dihydrobenzofuran-5-ylmethyl)-2-(methoxymethyl)pyrazolo[1,5-a]pyrimidine-3,7-dicarboxamide (28 mg) was synthesised following a similar procedure to that described in General Method E.

¹H NMR (400 MHz, DMSO-d₆) δ 10.20 (t, J=5.6 Hz, 1H), 8.92 (d, J=4.4 Hz, 1H), 7.75-7.68 (m, 2H), 7.59 (s, 1H), 7.34-7.29 (m, 1H), 7.16 (d, J=8.1 Hz, 1H), 6.73 (d, J=8.1 Hz, 1H), 4.97 (s, 2H), 4.58 (d, J=5.7 Hz, 2H), 4.51 (t, J=8.7 Hz, 2H), 3.35 (s, 3H), 3.16 (t, J=8.7 Hz, 2H)

LCMS (Method 2): 2.02 min, 382.2 [M+H]⁺

### Synthesis of Compound 68, N7-(2,4-difluorophenyl)-2-(methoxymethyl)pyrazolo[1,5-a]pyrimidine-3,7-dicarboxamide

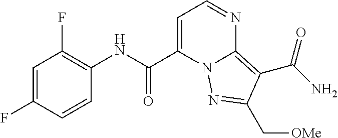 [SMILES: COCc1nn2c(C(=O)Nc3ccc(F)cc3F)ccnc2c1C(N)=O]

N7-(2,4-difluorophenyl)-2-(methoxymethyl)pyrazolo[1,5-a]pyrimidine-3,7-dicarboxamide (81 mg) was synthesised following a similar procedure to that described in General Method E.

¹H NMR (400 MHz, DMSO-d₆) δ 12.35 (s, 1H), 9.00 (d, J=4.4 Hz, 1H), 8.47-8.37 (m, 1H), 7.91 (d, J=4.4 Hz, 1H), 7.72 (s, 1H), 7.66 (s, 1H), 7.57-7.47 (m, 1H), 7.27-7.17 (m, 1H), 5.03 (s, 2H), 3.43 (s, 3H)

LCMS (Method 2): 2.215 min, 362.17 [M+H]⁺

### Synthesis of Compound 69, N7-benzyl-2-(methoxymethyl)pyrazolo[1,5-a]pyrimidine-3,7-dicarboxamide

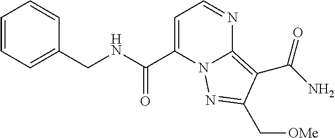 [SMILES: COCc1nn2c(C(=O)NCc3ccccc3)ccnc2c1C(N)=O]

N7-benzyl-2-(methoxymethyl)pyrazolo[1,5-a]pyrimidine-3,7-dicarboxamide (52 mg) was synthesised following a similar procedure to that described in General Method E.

¹H NMR (400 MHz, DMSO-d₆) δ 10.27 (t, J=5.8 Hz, 1H), 8.93 (d, J=4.4 Hz, 1H), 7.72-7.67 (m, 2H), 7.60 (s, 1H), 7.50-7.43 (m, 2H), 7.41-7.35 (m, 2H), 7.33-7.25 (m, 1H), 4.98 (s, 2H), 4.68 (d, J=5.8 Hz, 2H), 3.36 (s, 3H)

LCMS (Method 2): 2.02 min, 340.2 [M+H]⁺

### Synthesis of Compound 70, N7-[(4-fluorophenyl)methyl]-2-(methoxymethyl)pyrazolo[1,5-α]pyrimidine-3,7-dicarboxamide

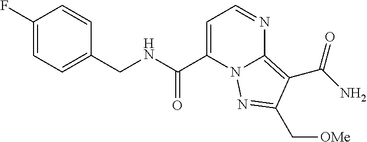 [SMILES: COCc1nn2c(C(=O)NCc3ccc(F)cc3)ccnc2c1C(N)=O]

N7-[(4-fluorophenyl)methyl]-2-(methoxymethyl)pyrazolo[1,5-a]pyrimidine-3,7-dicarboxamide (53 mg) was synthesised following a similar procedure to that described in General Method E.

¹H NMR (400 MHz, DMSO-d₆) δ 10.25 (t, J=5.9 Hz, 1H), 8.94 (d, J=4.4 Hz, 1H), 7.71 (d, J=4.4 Hz, 2H), 7.61 (s, 1H), 7.57-7.48 (m, 2H), 7.26-7.16 (m, 2H), 4.99 (s, 2H), 4.68 (d, J=5.8 Hz, 2H), 3.37 (s, 3H)

LCMS (Method 2): 2.04 min, 358.2 [M+H]⁺

### Synthesis of Compound 71, N7-[(3-fluorophenyl)methyl]-2-(methoxymethyl)pyrazolo[1,5-a]pyrimidine-3,7-dicarboxamide

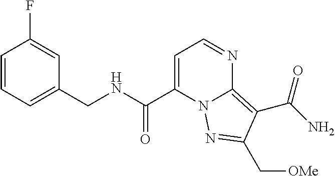 [SMILES: COCc1nn2c(C(=O)NCc3cccc(F)c3)ccnc2c1C(N)=O]

N7-[(3-fluorophenyl)methyl]-2-(methoxymethyl)pyrazolo[1,5-a]pyrimidine-3,7-dicarboxamide (63 mg) was synthesised following a similar procedure to that described in General Method E.

¹H NMR (400 MHz, DMSO-d₆) δ 10.27 (t, J=5.8 Hz, 1H), 8.94 (d, J=4.4 Hz, 1H), 7.71 (d, J=4.4 Hz, 2H), 7.61 (s, 1H), 7.48-7.38 (m, 1H), 7.37-7.29 (m, 2H), 7.16-7.09 (m, 1H), 4.99 (s, 2H), 4.71 (d, J=5.9 Hz, 2H), 3.38 (s, 3H)

LCMS (Method 2): 2.07 min, 358.2 [M+H]⁺

### Synthesis of Compound 72, 2-(methoxymethyl)-N7-(p-tolylmethyl)pyrazolo[1,5-a]pyrimidine-3,7-dicarboxamide

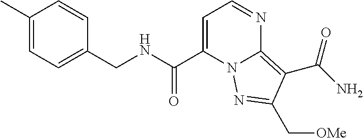 [SMILES: COCc1nn2c(C(=O)NCc3ccc(C)cc3)ccnc2c1C(N)=O]

2-(Methoxymethyl)-N7-(p-tolylmethyl)pyrazolo[1,5-a]pyrimidine-3,7-dicarboxamide (18 mg) was synthesised following a similar procedure to that described in General Method E.

¹H NMR (400 MHz, DMSO-d₆) δ 10.24 (s, 1H), 8.93 (d, J=4.4 Hz, 1H), 7.78-7.65 (m, 2H), 7.60 (s, 1H), 7.34 (d, J=8.0 Hz, 2H), 7.18 (d, J=7.8 Hz, 2H), 4.98 (s, 2H), 4.63 (s, 2H), 3.35 (s, 3H), 2.30 (s, 3H)

LCMS (Method 2): 2.17 min, 354.2 [M+H]⁺

### Synthesis of Compound 73, 2-(methoxymethyl)-N7-(7-methoxytetralin-2-yl)pyrazolo[1,5-a]pyrimidine-3,7-dicarboxamide

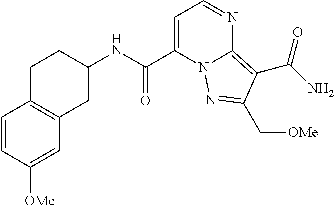 [SMILES: COCc1nn2c(C(=O)NC3CCc4ccc(OC)cc4C3)ccnc2c1C(N)=O]

2-(Methoxymethyl)-N7-(7-methoxytetralin-2-yl)pyrazolo[1,5-a]pyrimidine-3,7-dicarboxamide (23 mg) was synthesised following a similar procedure to that described in General Method E.

¹H NMR (400 MHz, DMSO-d₆) δ 10.17 (d, J=7.4 Hz, 1H), 8.92 (d, J=4.4 Hz, 1H), 7.73 (d, J=4.4 Hz, 1H), 7.67 (d, J=2.7 Hz, 1H), 7.58 (d, J=2.5 Hz, 1H), 7.04 (d, J=8.4 Hz, 1H), 6.72 (dd, J=8.4, 2.7 Hz, 1H), 6.68 (d, J=2.7 Hz, 1H), 4.89 (d, J=0.7 Hz, 2H), 4.48-4.34 (m, 1H), 3.70 (s, 3H), 3.33 (s, 3H), 3.15 (dd, J=16.7, 5.0 Hz, 1H), 2.99-2.88 (m, 1H), 2.88-2.76 (m, 2H), 2.14-2.01 (m, 1H), 1.99-1.86 (m, 1H)

LCMS (Method 2): 2.25 min, 410.2 [M+H]⁺

### Synthesis of Compound 74, 2-(methoxymethyl)-N7-[(1R)-tetralin-1-yl]pyrazolo[1,5-a]pyrimidine-3,7-dicarboxamide

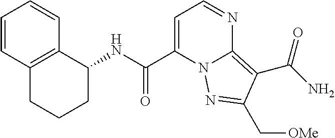 [SMILES: COCc1nn2c(C(=O)N[C@@H]3CCCc4ccccc43)ccnc2c1C(N)=O]

2-(Methoxymethyl)-N7-[(1R)-tetralin-1-yl]pyrazolo[1,5-a]pyrimidine-3,7-dicarboxamide (69 mg) was synthesised following a similar procedure to that described in General Method E.

¹H NMR (400 MHz, DMSO-d₆) δ 10.30 (d, J=8.3 Hz, 1H), 8.93 (d, J=4.4 Hz, 1H), 7.73 (d, J=4.4 Hz, 1H), 7.67 (s, 1H), 7.58 (s, 1H), 7.25-7.10 (m, 4H), 5.34-5.26 (m, 1H), 4.93 (s, 2H), 3.28 (s, 3H), 2.93-2.72 (m, 2H), 2.20-2.03 (m, 1H), 1.96-1.86 (m, 3H)

LCMS (Method 2): 2.32 min, 380.2 [M+H]⁺

### Synthesis of Compound 75, N7-(2,3-dihydrobenzofuran-3-yl)-2-(methoxymethyl)pyrazolo[1,5-a]pyrimidine-3,7-dicarboxamide

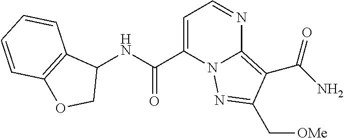 [SMILES: COCc1nn2c(C(=O)NC3COc4ccccc43)ccnc2c1C(N)=O]

N7-(2,3-dihydrobenzofuran-3-yl)-2-(methoxymethyl)pyrazolo[1,5-a]pyrimidine-3,7-dicarboxamide (115 mg) was synthesised following a similar procedure to that described in General Method E.

¹H NMR (400 MHz, DMSO-d₆) δ 10.38 (d, J=6.7 Hz, 1H), 8.93 (d, J=4.4 Hz, 1H), 7.71 (d, J=4.4 Hz, 1H), 7.66 (s, 1H), 7.59 (s, 1H), 7.52-7.46 (m, 1H), 7.33-7.25 (m, 1H), 7.01-6.94 (m, 1H), 6.92 (dd, J=8.2, 0.9 Hz, 1H), 5.82-5.72 (m, 1H), 4.97-4.87 (m, 2H), 4.83 (dd, J=9.9, 8.2 Hz, 1H), 4.47 (dd, J=9.9, 4.4 Hz, 1H), 3.26 (s, 3H)

LCMS (Method 2): 2.05 min, 368.2 [M+H]⁺

### Synthesis of Compound 76, N7-cyclohexyl-2-(methoxymethyl)pyrazolo[1,5-a]pyrimidine-3,7-dicarboxamide

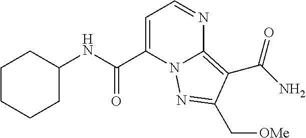 [SMILES: COCc1nn2c(C(=O)NC3CCCCC3)ccnc2c1C(N)=O]

N7-cyclohexyl-2-(methoxymethyl)pyrazolo[1,5-a]pyrimidine-3,7-dicarboxamide (29 mg) was synthesised following a similar procedure to that described in General Method E.

¹H NMR (400 MHz, DMSO-d₆) δ 10.10 (d, J=7.7 Hz, 1H), 8.92 (d, J=4.4 Hz, 1H), 7.71 (d, J=4.4 Hz, 1H), 7.69 (s, 1H), 7.60 (s, 1H), 5.01 (s, 2H), 4.07-3.89 (m, 1H), 3.44 (s, 3H), 1.98-1.82 (m, 2H), 1.81-1.66 (m, 2H), 1.60-1.49 (m, 1H), 1.50-1.26 (m, 5H)

LCMS (Method 2): 2.21 min, 332.2 [M+H]⁺

### Synthesis of Compound 77, N7-[(2,2-dimethylcyclopropyl)methyl]-2-(methoxymethyl)pyrazolo[1,5-a]pyrimidine-3,7-dicarboxamide

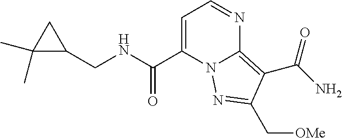 [SMILES: COCc1nn2c(C(=O)NCC3CC3(C)C)ccnc2c1C(N)=O]

N7-[(2,2-dimethylcyclopropyl)methyl]-2-(methoxymethyl)pyrazolo[1,5-a]pyrimidine-3,7-dicarboxamide (23 mg) was synthesised following a similar procedure to that described in General Method E.

¹H NMR (400 MHz, DMSO-d₆) δ 10.08 (t, J=5.4 Hz, 1H), 8.94 (d, J=4.4 Hz, 1H), 7.75 (d, J=4.4 Hz, 1H), 7.72 (s, 1H), 7.62 (s, 1H), 5.01 (s, 2H), 3.77-3.68 (m, 1H), 3.42 (s, 3H), 3.25-3.16 (m, 1H), 1.19 (s, 3H), 1.09 (s, 3H), 0.97-0.87 (m, 1H), 0.52 (dd, J=8.6, 4.3 Hz, 1H), 0.27 (t, J=4.8 Hz, 1H)

LCMS (Method 2): 2.19 min, 332.2 [M+H]⁺

### Synthesis of Compound 78, N7-chroman-3-yl-2-(methoxymethyl)pyrazolo[1,5-a]pyrimidine-3,7-dicarboxamide

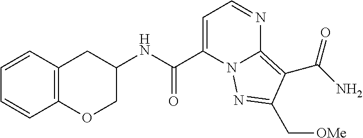 [SMILES: COCc1nn2c(C(=O)NC3COc4ccccc4C3)ccnc2c1C(N)=O]

N7-chroman-3-yl-2-(methoxymethyl)pyrazolo[1,5-a]pyrimidine-3,7-dicarboxamide (122 mg) was synthesised following a similar procedure to that described in General Method E.

¹H NMR (400 MHz, DMSO-d₆) δ 10.32 (d, J=7.5 Hz, 1H), 8.95-8.90 (m, 1H), 7.77 (d, J=4.4 Hz, 1H), 7.67 (s, 1H), 7.57 (s, 1H), 7.17-7.09 (m, 2H), 6.93-6.82 (m, 2H), 4.84-4.74 (m, 2H), 4.65-4.56 (m, 1H), 4.29-4.18 (m, 2H), 3.33-3.27 (m, 1H), 3.24 (s, 3H), 2.94-2.78 (m, 1H)

LCMS (Method 2): 2.09 min, 382.2 [M+H]⁺

### Synthesis of Compound 79, 2-(methoxymethyl)-N7-[(2S)-tetralin-2-yl]pyrazolo[1,5-a]pyrimidine-3,7-dicarboxamide

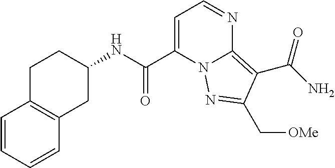 [SMILES: COCc1nn2c(C(=O)N[C@H]3CCc4ccccc4C3)ccnc2c1C(N)=O]

2-(Methoxymethyl)-N7-[(2S)-tetralin-2-yl]pyrazolo[1,5-a]pyrimidine-3,7-dicarboxamide (15 mg) was synthesised following a similar procedure to that described in General Method E.

¹H NMR (400 MHz, DMSO-d₆) δ 10.17 (d, J=7.4 Hz, 1H), 8.92 (d, J=4.4 Hz, 1H), 7.73 (d, J=4.4 Hz, 1H), 7.67 (d, J=2.5 Hz, 1H), 7.58 (s, 1H), 7.19-7.07 (m, 4H), 4.89 (s, 2H), 4.49-4.37 (m, 1H), 3.32 (s, 3H), 3.19 (dd, J=16.6, 5.1 Hz, 1H), 3.07-2.96 (m, 1H), 2.96-2.89 (m, 1H), 2.84 (dd, J=16.9, 6.9 Hz, 1H), 2.17-2.05 (m, 1H), 2.02-1.89 (m, 1H)

LCMS (Method 2): 2.28 min, 380.2 [M+H]⁺

### Synthesis of Compound 80, N7-(4,4-difluorocyclohexyl)-2-(methoxymethyl)pyrazolo[1,5-a]pyrimidine-3,7-dicarboxamide

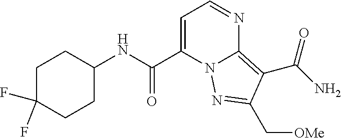 [SMILES: COCc1nn2c(C(=O)NC3CCC(F)(F)CC3)ccnc2c1C(N)=O]

N7-(4,4-difluorocyclohexyl)-2-(methoxymethyl)pyrazolo[1,5-a]pyrimidine-3,7-dicarboxamide (30 mg) was synthesised following a similar procedure to that described in General Method E.

¹H NMR (400 MHz, DMSO-d₆) δ 10.11 (d, J=7.5 Hz, 1H), 8.93 (d, J=4.4 Hz, 1H), 7.71-7.66 (m, 2H), 7.61 (s, 1H), 5.00 (s, 2H), 4.25-4.09 (m, 1H), 3.42 (s, 3H), 2.17-1.93 (m, 6H), 1.82-1.65 (m, 2H)

LCMS (Method 2): 2.02 min, 368.2 [M+H]⁺

### Synthesis of Compound 81, N7-(cyclopropylmethyl)-2-(methoxymethyl)pyrazolo[1,5-a]pyrimidine-3,7-dicarboxamide

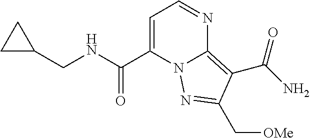 [SMILES: COCc1nn2c(C(=O)NCC3CC3)ccnc2c1C(N)=O]

N7-(cyclopropylmethyl)-2-(methoxymethyl)pyrazolo[1,5-a]pyrimidine-3,7-dicarboxamide (40 mg) was synthesised following a similar procedure to that described in General Method E.

¹H NMR (400 MHz, DMSO-d₆) δ 10.04 (t, J=5.5 Hz, 1H), 8.93 (d, J=4.4 Hz, 1H), 7.72-7.69 (m, 2H), 7.60 (d, J=2.5 Hz, 1H), 5.01 (s, 2H), 3.44 (s, 3H), 3.39-3.33 (m, 2H), 1.16-1.03 (m, 1H), 0.58-0.48 (m, 2H), 0.38-0.28 (m, 2H)

LCMS (Method 2): 1.82 min, 304.2 [M+H]⁺

### Synthesis of Compound 82, N7-(7-fluorotetralin-2-yl)-2-(methoxymethyl)pyrazolo[1,5-a]pyrimidine-3,7-dicarboxamide

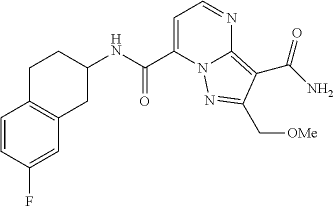 [SMILES: COCc1nn2c(C(=O)NC3CCc4ccc(F)cc4C3)ccnc2c1C(N)=O]

N7-(7-fluorotetralin-2-yl)-2-(methoxymethyl)pyrazolo[1,5-a]pyrimidine-3,7-dicarboxamide (27 mg) was synthesised following a similar procedure to that described in General Method E.

¹H NMR (400 MHz, DMSO-d₆) δ 10.16 (d, J=7.4 Hz, 1H), 8.92 (d, J=4.4 Hz, 1H), 7.72 (d, J=4.4 Hz, 1H), 7.67 (d, J=2.6 Hz, 1H), 7.59 (s, 1H), 7.22-7.13 (m, 1H), 7.02-6.92 (m, 2H), 4.89 (s, 2H), 4.52-4.31 (m, 1H), 3.33 (s, 3H), 3.19 (dd, J=17.0, 5.1 Hz, 1H), 3.05-2.93 (m, 1H), 2.93-2.81 (m, 2H), 2.16-2.03 (m, 1H), 2.02-1.88 (m, 1H)

LCMS (Method 2): 2.27 min, 398.2 [M+H]⁺

### Synthesis of Compound 83, N7-[(2,4-difluorophenyl)methyl]-2-(methoxymethyl)pyrazolo[1,5-a]pyrimidine-3,7-dicarboxamide

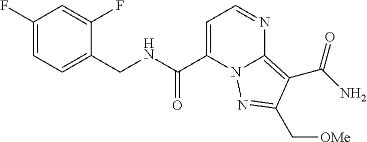 [SMILES: COCc1nn2c(C(=O)NCc3ccc(F)cc3F)ccnc2c1C(N)=O]

N7-[(2,4-difluorophenyl)methyl]-2-(methoxymethyl)pyrazolo[1,5-a]pyrimidine-3,7-dicarboxamide (25 mg) was synthesised following a similar procedure to that described in General Method E.

¹H NMR (400 MHz, DMSO-d₆) δ 10.31-10.27 (m, 1H), 8.93 (d, J=4.4 Hz, 1H), 7.73-7.69 (m, 2H), 7.69-7.62 (m, 1H), 7.61 (s, 1H), 7.30 (ddd, J=10.5, 9.3, 2.6 Hz, 1H), 7.11 (ddd, J=8.5, 2.6, 1.1 Hz, 1H), 4.99 (s, 2H), 4.69 (d, J=5.0 Hz, 2H), 3.38 (s, 3H)

LCMS (Method 2): 2.11 min, 376.2 [M+H]⁺

### Synthesis of Compound 84, 2-(methoxymethyl)-N7-(cis-4-methylcyclohexyl)pyrazolo[1,5-a]pyrimidine-3,7-dicarboxamide

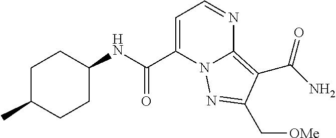 [SMILES: COCc1nn2c(C(=O)N[C@H]3CC[C@@H](C)CC3)ccnc2c1C(N)=O]

2-(Methoxymethyl)-N7-(cis-4-methylcyclohexyl)pyrazolo[1,5-a]pyrimidine-3,7-dicarboxamide (47 mg) was synthesised following a similar procedure to that described in General Method E.

¹H NMR (400 MHz, DMSO-d₆) δ 10.15 (d, J=7.9 Hz, 1H), 8.64 (d, J=4.4 Hz, 1H), 7.49 (d, J=4.4 Hz, 1H), 7.42 (s, 1H), 7.32 (s, 1H), 4.73 (s, 2H), 4.00-3.93 (m, 1H), 3.12 (s, 3H), 1.52-1.44 (m, 2H), 1.40-1.25 (m, 4H), 1.23-1.15 (m, 1H), 1.06-0.94 (m, 2H), 0.65 (d, J=6.4 Hz, 3H)

LCMS (Method 2): 2.33 min, 346.2 [M+H]⁺

### Synthesis of Compound 85, N7-(5,6-dihydro-4H-cyclopenta[c]thiophen-5-yl)-2-(methoxymethyl)pyrazolo[1,5-a]pyrimidine-3,7-dicarboxamide

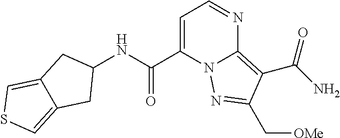 [SMILES: COCc1nn2c(C(=O)NC3Cc4cscc4C3)ccnc2c1C(N)=O]

N7-(5,6-dihydro-4H-cyclopenta[c]thiophen-5-yl)-2-(methoxymethyl)pyrazolo[1,5-a]pyrimidine-3,7-dicarboxamide (33 mg) was synthesised following a similar procedure to that described in General Method E.

¹H NMR (400 MHz, DMSO-d₆) δ 10.25 (d, J=7.3 Hz, 1H), 8.92 (d, J=4.4 Hz, 1H), 7.70-7.65 (m, 2H), 7.58 (s, 1H), 7.12 (d, J=0.9 Hz, 2H), 5.05-4.96 (m, 1H), 4.94 (s, 2H), 3.36 (s, 3H), 3.24-3.13 (m, 2H), 2.77-2.67 (m, 2H)

LCMS (Method 2): 2.19 min, 372.1 [M+H]⁺

### Synthesis of Compound 86, 2-(methoxymethyl)-N7-(trans-3-methylcyclobutyl)pyrazolo[1,5-a]pyrimidine-3,7-dicarboxamide

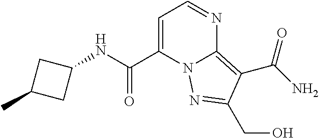 [SMILES: C[C@H]1C[C@H](NC(=O)c2ccnc3c(C(N)=O)c(CO)nn23)C1]

2-(Methoxymethyl)-N7-(trans-3-methylcyclobutyl)pyrazolo[1,5-a]pyrimidine-3,7-dicarboxamide (10 mg) was synthesised following a similar procedure to that described in General Method E.

¹H NMR (400 MHz, DMSO-d₆) δ 10.15 (d, J=7.1 Hz, 1H), 8.91 (d, J=4.4 Hz, 1H), 7.71-7.62 (m, 2H), 7.60 (s, 1H), 5.01 (s, 2H), 4.64-4.50 (m, 1H), 3.44 (s, 3H), 2.42 (s, 1H), 2.26-2.15 (m, 2H), 2.12-2.00 (m, 2H), 1.16 (s, 3H)

LCMS (Method 2): 2.04 min, 318.2 [M+H]⁺

### Synthesis of Compound 87, N7-(5-chloroindan-2-yl)-2-(methoxymethyl)pyrazolo[1,5-a]pyrimidine-3,7-dicarboxamide

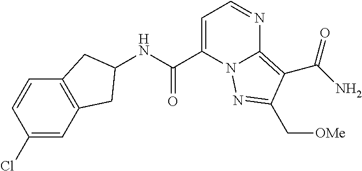 [SMILES: COCc1nn2c(C(=O)NC3Cc4ccc(Cl)cc4C3)ccnc2c1C(N)=O]

N7-(5-chloroindan-2-yl)-2-(methoxymethyl)pyrazolo[1,5-a]pyrimidine-3,7-dicarboxamide (21 mg) was synthesised following a similar procedure to that described in General Method E.

¹H NMR (400 MHz, DMSO-d₆) δ 10.19 (d, J=7.1 Hz, 1H), 8.91 (d, J=4.4 Hz, 1H), 7.71-7.65 (m, 2H), 7.60-7.55 (m, 1H), 7.37 (d, J=2.0 Hz, 1H), 7.31 (d, J=8.1 Hz, 1H), 7.27-7.20 (m, 1H), 4.92 (s, 2H), 4.86-4.76 (m, 1H), 3.45-3.34 (m, 2H), 3.31 (s, 3H), 3.03-2.90 (m, 2H)

LCMS (Method 2): 2.32 min, 400.14 [M+H]⁺

### Synthesis of Compound 88, N7-(7-bicyclo[4.2.0]octa-1,3,5-trienyl)-2-(methoxymethyl)pyrazolo[1,5-a]pyrimidine-3,7-dicarboxamide

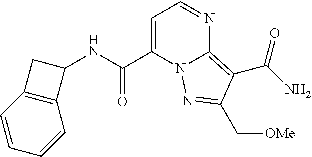 [SMILES: COCc1nn2c(C(=O)NC3Cc4ccccc43)ccnc2c1C(N)=O]

N7-(7-bicyclo[4.2.0]octa-1,3,5-trienyl)-2-(methoxymethyl)pyrazolo[1,5-a]pyrimidine-3,7-dicarboxamide (23 mg) was synthesised following a similar procedure to that described in General Method E.

¹H NMR (400 MHz, DMSO-d₆) δ 10.52 (d, J=6.0 Hz, 1H), 8.94 (d, J=4.4 Hz, 1H), 7.72 (d, J=4.4 Hz, 1H), 7.68 (s, 1H), 7.60 (s, 1H), 7.40-7.21 (m, 4H), 5.56 (s, 1H), 4.96 (d, J=1.3 Hz, 2H), 3.83-3.73 (m, 1H), 3.34 (s, 3H), 3.23-3.15 (m, 1H)

LCMS (Method 2): 2.34, 352.2 [M+H]⁺

### Synthesis of Compound 89, 2-(methoxymethyl)-7-[4-[3-(trifluoromethyl)-2-pyridyl]piperazine-1-carbonyl]pyrazolo[1,5-a]pyrimidine-3-carboxamide

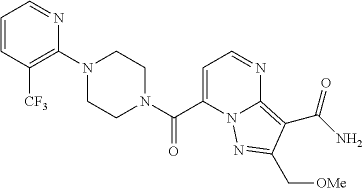 [SMILES: COCc1nn2c(C(=O)N3CCN(c4ncccc4C(F)(F)F)CC3)ccnc2c1C(N)=O]

2-(Methoxymethyl)-7-[4-[3-(trifluoromethyl)-2-pyridyl]piperazine-1-carbonyl]pyrazolo[1,5-a]pyrimidine-3-carboxamide (53 mg) was synthesised following a similar procedure to that described in General Method E.

¹H NMR (400 MHz, DMSO-d₆) δ 8.87 (d, J=4.3 Hz, 1H), 8.60-8.53 (m, 1H), 8.11 (dd, J=7.8, 1.8 Hz, 1H), 7.67 (s, 1H), 7.55 (s, 1H), 7.43 (d, J=4.3 Hz, 1H), 7.30-7.22 (m, 1H), 4.94 (d, J=9.4 Hz, 2H), 3.88 (d, J=16.4 Hz, 2H), 3.41-3.33 (m, 7H), 3.16 (d, J=5.2 Hz, 2H)

LCMS (Method 2): 2.11, 464.2 [M+H]⁺

### Synthesis of Compound 90, N7-[[1-(dimethylamino)cyclohexyl]methyl]-2-(methoxymethyl)pyrazolo[1,5-a]pyrimidine-3,7-dicarboxamide

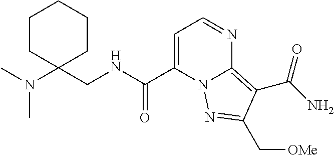 [SMILES: COCc1nn2c(C(=O)NCC3(N(C)C)CCCCC3)ccnc2c1C(N)=O]

N7-[[1-(dimethylamino)cyclohexyl]methyl]-2-(methoxymethyl)pyrazolo[1,5-a]pyrimidine-3,7-dicarboxamide (11 mg) was synthesised following a similar procedure to that described in General Method E.

¹H NMR (400 MHz, DMSO-d₆) δ 10.22 (t, J=5.4 Hz, 1H), 8.93 (d, J=4.4 Hz, 1H), 7.77 (d, J=4.4 Hz, 1H), 7.72 (s, 1H), 7.61 (s, 1H), 4.98 (s, 2H), 3.56 (d, J=5.5 Hz, 2H), 3.39 (s, 3H), 2.29 (s, 6H), 1.72-1.31 (m, 10H).

LCMS (Method 2): 1.32 min, 389.2 [M+H]⁺

### Synthesis of Compound 91, N7-(4-fluoroindan-2-yl)-2-(methoxymethyl)pyrazolo[1,5-a]pyrimidine-3,7-dicarboxamide

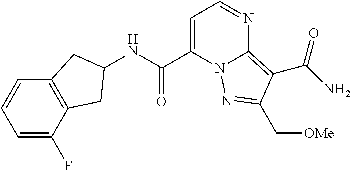 [SMILES: COCc1nn2c(C(=O)NC3Cc4cccc(F)c4C3)ccnc2c1C(N)=O]

N7-(4-fluoroindan-2-yl)-2-(methoxymethyl)pyrazolo[1,5-a]pyrimidine-3,7-dicarboxamide (10 mg) was synthesised following a similar procedure to that described in General Method E.

¹H NMR (400 MHz, DMSO-d₆) δ 10.21 (d, J=7.1 Hz, 1H), 8.91 (d, J=4.4 Hz, 1H), 7.73-7.61 (m, 2H), 7.58 (s, 1H), 7.29-7.20 (m, 1H), 7.15 (d, J=7.4 Hz, 1H), 7.06-6.98 (m, 1H), 4.91 (s, 2H), 4.91-4.82 (m, 1H), 3.49-3.37 (m, 2H), 3.31 (s, 3H), 3.02 (dt, 2H)

LCMS (Method 7): 2.45 min, 384.2 [M+H]⁺

### Synthesis of Compound 92, N7-(4,6-difluoroindan-2-yl)-2-(methoxymethyl)pyrazolo[1,5-a]pyrimidine-3,7-dicarboxamide

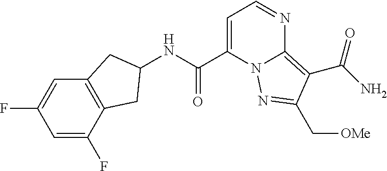 [SMILES: COCc1nn2c(C(=O)NC3Cc4cc(F)cc(F)c4C3)ccnc2c1C(N)=O]

N7-(4,6-difluoroindan-2-yl)-2-(methoxymethyl)pyrazolo[1,5-a]pyrimidine-3,7-dicarboxamide (10 mg) was synthesised following a similar procedure to that described in General Method E.

¹H NMR (400 MHz, DMSO-d₆) δ 10.21 (d, J=7.1 Hz, 1H), 8.91 (d, J=4.4 Hz, 1H), 7.67 (t, J=4.3 Hz, 2H), 7.58 (s, 1H), 7.11-7.00 (m, 2H), 4.92 (s, 2H), 4.91-4.82 (m, 1H), 3.48-3.32 (m, 5H), 3.07-2.92 (m, 2H).

LCMS (Method 7): 2.52 min, 402.2 [M+H]⁺

### Synthesis of tert-butyl 3-[[3-carbamoyl-2-(methoxymethyl)pyrazolo[1,5-a]pyrimidine-7-carbonyl]amino]-3,4-dihydro-2H-quinoline-1-carboxylate

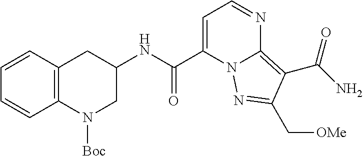 [SMILES: COCc1nn2c(C(=O)NC3Cc4ccccc4N(C(=O)OC(C)(C)C)C3)ccnc2c1C(N)=O]

Tert-butyl 3-[[3-carbamoyl-2-(methoxymethyl)pyrazolo[1,5-a]pyrimidine-7-carbonyl]amino]-3,4-dihydro-2H-quinoline-1-carboxylate (197 mg) was synthesised following a similar procedure to that described in General Method E.

¹H NMR (400 MHz, CDCl₃) δ 10.55 (d, J=7.4 Hz, 1H), 8.79 (d, J=4.4 Hz, 1H), 8.04-7.91 (m, 2H), 7.78-7.71 (m, 1H), 7.25-7.15 (m, 2H), 7.11-7.03 (m, 1H), 5.64 (s, 1H), 5.02 (d, J=13.7 Hz, 1H), 4.86 (d, J=13.6 Hz, 1H), 4.79-4.69 (m, 1H), 4.15-4.05 (m, 1H), 3.90 (dd, J=13.0, 3.6 Hz, 1H), 3.36-3.26 (m, 4H), 2.98 (dd, J=16.5, 5.2 Hz, 1H), 1.36 (s, 9H)

LCMS (Method 2): 2.43 min, 481.2 [M+H]⁺

### Synthesis of Compound 93, 2-(methoxymethyl)-N7-(1,2,3,4-tetrahydroquinolin-3-yl)pyrazolo[1,5-a]pyrimidine-3,7-dicarboxamide

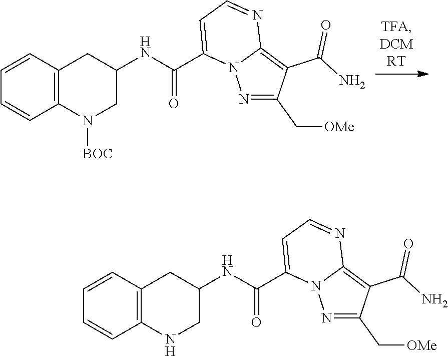 [SMILES: COCc1nn2c(C(=O)NC3Cc4ccccc4N(C(=O)OC(C)(C)C)C3)ccnc2c1C(N)=O ; COCc1nn2c(C(=O)NC3CNc4ccccc4C3)ccnc2c1C(N)=O]

TFA (250 μL, 3.26 mmol) was added to a solution of tert-butyl 3-[3-carbamoyl-2-(methoxymethyl)pyrazolo[1,5-a]pyrimidine-7-carbonyl]amino]-3,4-dihydro-2H-quinoline-1-carboxylate (100 mg, 0.21 mmol) in DCM (2 mL) and the reaction was stirred at room temperature for 5 h. The reaction mixture was concentrated in vacuo and the residue was azeotroped with DCM (×3). 2 M NH₃ in MeOH (20 mL) was added and the mixture was concentrated in vacuo (×2). The crude product was purified by column chromatography (eluting with 0 to 80% MeOH in DCM) to provide the title compound (71 mg).

¹H NMR (400 MHz, DMSO-d₆) δ 10.31 (d, J=7.6 Hz, 1H), 8.92 (d, J=4.3 Hz, 1H), 7.78 (d, J=4.4 Hz, 1H), 7.71-7.63 (m, 1H), 7.56 (s, 1H), 6.95-6.87 (m, 2H), 6.55 (dd, J=8.0, 1.1 Hz, 1H), 6.53-6.46 (m, 1H), 5.88 (s, 1H), 4.84-4.72 (m, 2H), 4.50 (s, 1H), 3.41 (d, J=11.6 Hz, 1H), 3.27 (s, 4H), 3.12 (dd, J=16.4, 4.9 Hz, 1H), 2.84-2.74 (m, 1H)

LCMS (Method 2): 2.00 min, 381.2 [M+H]⁺

### Synthesis of Compound 95, 7-(isoindoline-2-carbonyl)-2-(methoxymethyl)pyrazolo[1,5-a]pyrimidine-3-carboxamide

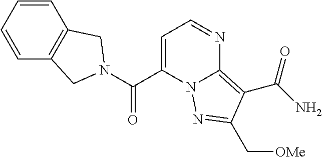 [SMILES: COCc1nn2c(C(=O)N3Cc4ccccc4C3)ccnc2c1C(N)=O]

7-(Isoindoline-2-carbonyl)-2-(methoxymethyl)pyrazolo[1,5-a]pyrimidine-3-carboxamide (30 mg) was synthesised following a similar procedure to that described in General Method E.

¹H NMR (400 MHz, DMSO-d₆) δ 8.92 (d, J=4.3 Hz, 1H), 7.68 (s, 1H), 7.58 (s, 1H), 7.48 (d, J=4.3 Hz, 1H), 7.45 (d, J=7.5 Hz, 1H), 7.37-7.26 (m, 2H), 7.19 (d, J=7.4 Hz, 1H), 4.98 (s, 2H), 4.91 (s, 2H), 4.62 (s, 2H), 3.30 (s, 3H)

LCMS (Method 7): 2.00 min, 352.2 [M+H]⁺

### Synthesis of Compound 111, 2-cyclopropyl-N7-[cis-3-(trifluoromethoxy)cyclobutyl]pyrazolo[1,5-a]pyrimidine-3,7-dicarboxamide

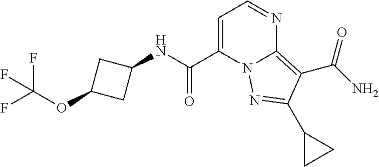 [SMILES: NC(=O)c1c(C2CC2)nn2c(C(=O)N[C@H]3C[C@@H](OC(F)(F)F)C3)ccnc12]

2-Cyclopropyl-N7-[cis-3-(trifluoromethoxy)cyclobutyl]pyrazolo[1,5-a]pyrimidine-3,7-dicarboxamide (23 mg) was synthesised following a similar procedure to that described in General Method E.

¹H NMR (400 MHz, DMSO-d₆) δ 9.91 (d, J=7.4 Hz, 1H), 8.84 (d, J=4.4 Hz, 1H), 7.80-7.72 (m, 1H), 7.59-7.50 (m, 2H), 4.71 (p, J=7.1 Hz, 1H), 4.25-4.11 (m, 1H), 3.26-3.15 (m, 1H), 2.96-2.85 (m, 2H), 2.33-2.22 (m, 2H), 1.20-1.03 (m, 4H)

LCMS (Method 4): 2.41 min, 384.0 [M+H]⁺

### Synthesis of Compound 112, 2-cyclopropyl-N7-(3,3-difluoroindan-1-yl)pyrazolo[1,5-a]pyrimidine-3,7-dicarboxamide

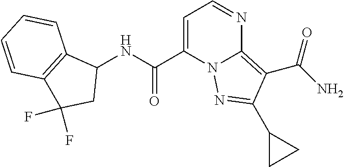 [SMILES: NC(=O)c1c(C2CC2)nn2c(C(=O)NC3CC(F)(F)c4ccccc43)ccnc12]

2-Cyclopropyl-N7-(3,3-difluoroindan-1-yl)pyrazolo[1,5-a]pyrimidine-3,7-dicarboxamide (36 mg) was synthesised following a similar procedure to that described in General Method E.

¹H NMR (400 MHz, DMSO-d₆) δ 10.25 (d, J=7.5 Hz, 1H), 8.86 (d, J=4.4 Hz, 1H), 7.75 (s, 1H), 7.72-7.62 (m, 4H), 7.62-7.56 (m, 1H), 7.54 (s, 1H), 5.74-5.64 (m, 1H), 3.30-3.11 (m, 2H), 2.70-2.56 (m, 1H), 1.10-0.98 (m, 2H), 0.89-0.79 (m, 2H)

LCMS (Method 7): 2.67 min, 398.2 [M+H]⁺

### Synthesis of Compound 113, 2-cyclopropyl-N7-[(4S)-7-fluorochroman-4-yl]pyrazolo[1,5-a]pyrimidine-3,7-dicarboxamide

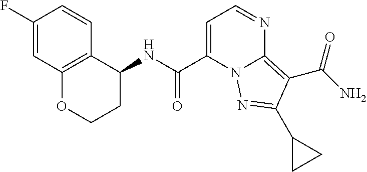 [SMILES: NC(=O)c1c(C2CC2)nn2c(C(=O)N[C@H]3CCOc4cc(F)ccc43)ccnc12]

2-Cyclopropyl-N7-[(4S)-7-fluorochroman-4-yl]pyrazolo[1,5-a]pyrimidine-3,7-dicarboxamide (56 mg) was synthesised following a similar procedure to that described in General Method E.

¹H NMR (400 MHz, DMSO-d₆) δ 10.11 (d, J=7.5 Hz, 1H), 8.85 (d, J=4.4 Hz, 1H), 7.75 (d, J=2.7 Hz, 1H), 7.63 (d, J=4.4 Hz, 1H), 7.53 (s, 1H), 7.47-7.39 (m, 1H), 6.86-6.66 (m, 2H), 5.28-5.19 (m, 1H), 4.41-4.31 (m, 1H), 4.26-4.16 (m, 1H), 3.21-3.09 (m, 1H), 2.31-2.19 (m, 1H), 2.16-2.04 (m, 1H), 1.09-1.00 (m, 2H), 0.83-0.73 (m, 2H)

LCMS (Method 7): 2.67 min, 396.2 [M+H]⁺

### Synthesis of N-chroman-3-yl-2,2,2-trifluoro-acetamide

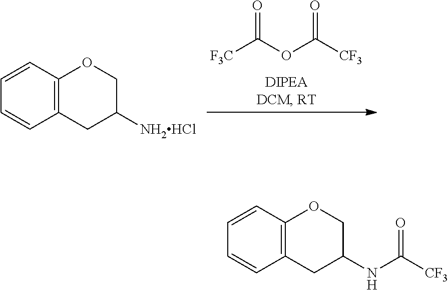

DIPEA (840 μL, 4.85 mmol) and trifluoroacetic anhydride (270 μL, 1.94 mmol) were added to a suspension of chroman-3-amine hydrochloride (300 mg, 1.62 mmol) in DCM (16 mL), and the reaction was stirred at room temperature for 2 h. The reaction mixture was concentrated in vacuo and the crude product was purified by column chromatography (eluting with 0 to 1% MeOH in DCM) to provide the title compound (210 mg).

¹H NMR (400 MHz, CDCl₃) δ 7.22-7.12 (m, 1H), 7.07 (d, J=7.5 Hz, 1H), 6.95 (td, J=7.4, 1.2 Hz, 1H), 6.89 (dd, J=8.3, 1.2 Hz, 1H), 6.60 (s, 1H), 4.61-4.49 (m, 1H), 4.29-4.20 (m, 1H), 4.19-4.08 (m, 1H), 3.22 (dd, J=17.1, 5.4 Hz, 1H), 2.90-2.77 (m, 1H)

LCMS (Method 7): 2.52 min, 244.1 [M−H]⁻

### Synthesis of N-(6-chlorochroman-3-yl)-2,2,2-trifluoro-acetamide

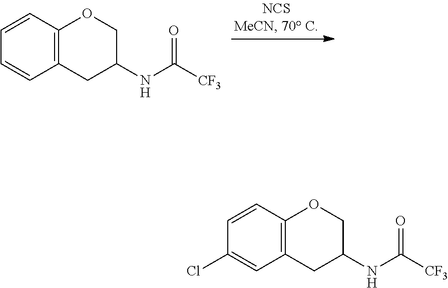 [SMILES: O=C(NC1COc2ccccc2C1)C(F)(F)F ; O=C(NC1COc2ccc(Cl)cc2C1)C(F)(F)F]

NCS (137 mg, 1.03 mmol) was added to a solution of N-chroman-3-yl-2,2,2-trifluoro-acetamide (210 mg, 0.86 mmol) in MeCN (5 mL), and the reaction was heated at 70° C. for 6 h. The reaction mixture was cooled to room temperature, diluted with EtOAc, and washed with sat. aq. NaHCO₃. The organic layer was dried over Na₂SO₄ and concentrated in vacuo to provide the title compound (249 mg).

¹H NMR (400 MHz, CDCl₃) δ 7.15-7.09 (m, 1H), 7.06 (dd, J=2.4, 1.1 Hz, 1H), 6.83 (d, J=8.7 Hz, 1H), 6.58 (s, 1H), 4.57-4.48 (m, 1H), 4.28-4.21 (m, 1H), 4.14-4.10 (m, 1H), 3.23-3.13 (m, 1H), 2.86-2.79 (m, 1H)

LCMS (Method 7): 2.82 min, 278.1 [M−H]⁻

### Synthesis of 6-chlorochroman-3-amine

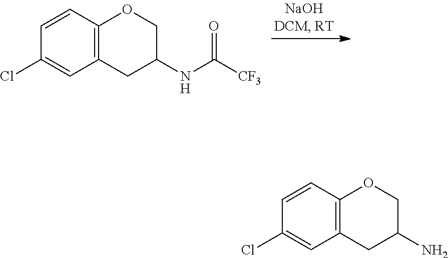 [SMILES: O=C(NC1COc2ccc(Cl)cc2C1)C(F)(F)F ; NC1COc2ccc(Cl)cc2C1]

4 M aq. NaOH (2.23 mL, 8.9 mmol) was added to a solution of N-(6-chlorochroman-3-yl)-2,2,2-trifluoro-acetamide (249 mg, 0.89 mmol) in DCM (2.2 mL) and the reaction was stirred at room temperature for 17 h. The reaction mixture was diluted with DCM and water, and the layers were separated. The organic layer was dried over Na₂SO₄ and concentrated in vacuo to provide the title compound (128 mg).

¹H NMR (400 MHz, CDCl₃) δ 7.09-7.01 (m, 2H), 6.76 (d, J=8.5 Hz, 1H), 4.19-4.07 (m, 1H), 3.90-3.77 (m, 1H), 3.38 (dt, J=9.8, 7.1 Hz, 1H), 3.09-2.94 (m, 1H), 2.57 (dd, J=16.3, 7.0 Hz, 1H). Exchangeable protons not observed.

LCMS (Method 7): 1.05 min, 184.3 [M+H]⁺

### Synthesis of Compound 114, N7-(6-chlorochroman-3-yl)-2-(methoxymethyl)pyrazolo[1,5-a]pyrimidine-3,7-dicarboxamide

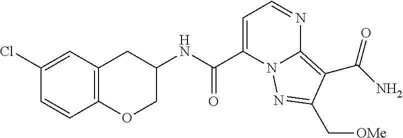 [SMILES: COCc1nn2c(C(=O)NC3COc4ccc(Cl)cc4C3)ccnc2c1C(N)=O]

N7-(6-chlorochroman-3-yl)-2-(methoxymethyl)pyrazolo[1,5-a]pyrimidine-3,7-dicarboxamide (83 mg) was synthesised following a similar procedure to that described in General Method E.

¹H NMR (400 MHz, DMSO-d₆) δ 10.30 (d, J=7.3 Hz, 1H), 8.92 (d, J=4.3 Hz, 1H), 7.76 (d, J=4.4 Hz, 1H), 7.67 (s, 1H), 7.58 (s, 1H), 7.24 (d, J=2.6 Hz, 1H), 7.17 (dd, J=8.7, 2.7 Hz, 1H), 6.88 (d, J=8.7 Hz, 1H), 4.87-4.73 (m, 2H), 4.65-4.55 (m, 1H), 4.30-4.22 (m, 2H), 3.30-3.28 (m, 1H), 3.26 (s, 3H), 2.88 (dd, J=17.1, 3.9 Hz, 1H)

LCMS (Method 7): 2.45 min, 416.1 [M+H]⁺

### Synthesis of 115, 2-cyclopropyl-N7-(6,7-dihydro-5H-cyclopenta[b]pyridin-6-yl)pyrazolo[1,5-a]pyrimidine-3,7-dicarboxamide

2-Cyclopropyl-N7-(6,7-dihydro-5H-cyclopenta[b]pyridin-6-yl)pyrazolo[1,5-a]pyrimidine-3,7-dicarboxamide (28 mg) was synthesised following a similar procedure to that described in General Method E.

¹H NMR (400 MHz, DMSO-d₆) δ 10.18 (d, J=7.3 Hz, 1H), 8.85 (d, J=4.4 Hz, 1H), 8.41-8.35 (m, 1H), 7.77-7.68 (m, 2H), 7.65 (d, J=4.4 Hz, 1H), 7.53 (s, 1H), 7.21 (dd, J=7.6, 5.0 Hz, 1H), 4.88-4.76 (m, 1H), 3.49-3.38 (m, 2H), 3.20-3.07 (m, 1H), 2.97 (ddd, J=16.7, 8.6, 3.7 Hz, 2H), 1.07-0.98 (m, 2H), 0.78-0.67 (m, 2H)

LCMS (Method 4): 1.33 min, 363.2 [M+H]⁺

### Synthesis of Compound 116, N7-(4,4-difluorotetralin-1-yl)-2-(methoxymethyl)pyrazolo[1,5-a]pyrimidine-3,7-dicarboxamide

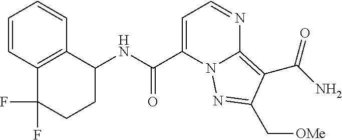 [SMILES: COCc1nn2c(C(=O)NC3CCC(F)(F)c4ccccc43)ccnc2c1C(N)=O]

N7-(4,4-difluorotetralin-1-yl)-2-(methoxymethyl)pyrazolo[1,5-a]pyrimidine-3,7-dicarboxamide (168 mg) was synthesised following a similar procedure to that described in General Method E.

¹H NMR (400 MHz, DMSO-d₆) δ 10.26 (d, J=8.3 Hz, 1H), 8.93 (d, J=4.4 Hz, 1H), 7.72-7.63 (m, 4H), 7.62-7.46 (m, 3H), 5.50-5.35 (m, 1H), 4.95 (s, 2H), 3.31 (s, 3H), 2.59-2.51 (m, 1H), 2.46 (s, 1H), 2.37-2.27 (m, 1H), 2.14-2.01 (m, 1H)

LCMS (Method 7): 2.50 min, 416.2 [M+H]⁺

### Synthesis of Compounds 175 and 176, N7-[-4,4-difluorotetralin-1-yl]-2-(methoxymethyl)pyrazolo[1,5-a]pyrimidine-3,7-dicarboxamide

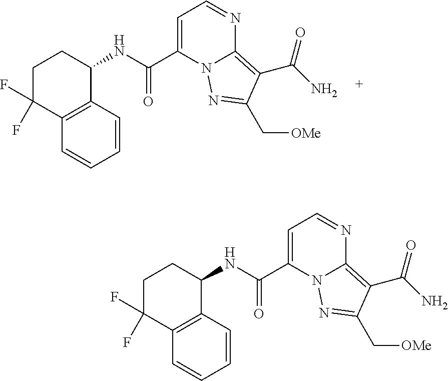 [SMILES: COCc1nn2c(C(=O)N[C@H]3CCC(F)(F)c4ccccc43)ccnc2c1C(N)=O ; COCc1nn2c(C(=O)N[C@@H]3CCC(F)(F)c4ccccc43)ccnc2c1C(N)=O]

Racemic N7-[-4,4-difluorotetralin-1-yl]-2-(methoxymethyl)pyrazolo[1,5-a]pyrimidine-3,7-dicarboxamide (40 mg) was separated by preparative SFC (eluting with 35% MeOH in CO₂) to provide enantiomer 1 (16 mg) and enantiomer 2 (14 mg) with unknown absolute configurations. Enantiomer 1 has a retention time of 2.46 min and enantiomer 2 has a retention time of 3.88 min.

¹H NMR (400 MHz, MeOD) δ 8.93 (d, J=4.4 Hz, 1H), 7.90 (d, J=4.4 Hz, 1H), 7.75-7.68 (m, 1H), 7.62-7.55 (m, 1H), 7.54-7.42 (m, 2H), 5.50 (s, 1H), 5.08-4.93 (m, 2H), 3.34 (s, 3H), 2.69-2.33 (m, 3H), 2.31-2.18 (m, 1H). Exchangeable protons not observed.

LCMS (Method 7) 2.53 min; 416.2 [M+H]⁺

### Synthesis of Compound 117, N7-(8-fluoro-3-quinolyl)-2-(methoxymethyl)pyrazolo[1,5-a]pyrimidine-3,7-dicarboxamide

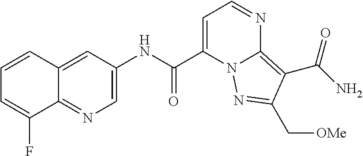 [SMILES: COCc1nn2c(C(=O)Nc3cnc4c(F)cccc4c3)ccnc2c1C(N)=O]

N7-(8-fluoro-3-quinolyl)-2-(methoxymethyl)pyrazolo[1,5-a]pyrimidine-3,7-dicarboxamide (40 mg) was synthesised following a similar procedure to that described in General Method E.

¹H NMR (400 MHz, DMSO-d₆) δ 12.31 (s, 1H), 9.07-8.98 (m, 3H), 7.92 (d, J=8.2 Hz, 1H), 7.81 (d, J=4.4 Hz, 1H), 7.71 (s, 1H), 7.69-7.61 (m, 2H), 7.61-7.51 (m, 1H), 5.04 (s, 2H), 3.45 (s, 3H)

LCMS (Method 7): 2.32 min, 395.2 [M+H]⁺

### Synthesis of Compound 118, 2-(methoxymethyl)-N7-(1-methylindol-2-yl)pyrazolo[1,5-a]pyrimidine-3,7-dicarboxamide

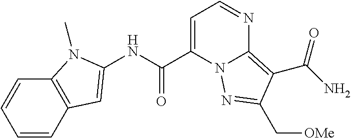 [SMILES: COCc1nn2c(C(=O)Nc3cc4ccccc4n3C)ccnc2c1C(N)=O]

2-(Methoxymethyl)-N7-(1-methylindol-2-yl)pyrazolo[1,5-a]pyrimidine-3,7-dicarboxamide (15 mg) was synthesised following a similar procedure to that described in General Method E.

¹H NMR (400 MHz, DMSO-d₆) δ 12.95 (s, 1H), 9.02 (d, J=4.4 Hz, 1H), 7.96 (d, J=4.4 Hz, 1H), 7.71 (d, J=11.8 Hz, 2H), 7.54 (d, J=7.8 Hz, 1H), 7.48 (d, J=8.1 Hz, 1H), 7.20-7.11 (m, 1H), 7.05 (t, J=7.4 Hz, 1H), 6.94 (s, 1H), 5.12 (s, 2H), 3.93 (s, 3H), 3.49 (s, 3H)

LCMS (Method 7): 2.73 min, 379.2 [M+H]⁺

### Synthesis of Compound 119, 2-(methoxymethyl)-N7-[(3R)-6,7-difluoro-2,3-dihydrobenzofuran-3-yl]pyrazolo[1,5-a]pyrimidine-3,7-dicarboxamide

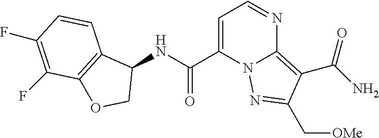 [SMILES: COCc1nn2c(C(=O)N[C@H]3COc4c3ccc(F)c4F)ccnc2c1C(N)=O]

2-(Methoxymethyl)-N7-[(3R)-6,7-difluoro-2,3-dihydrobenzofuran-3-yl]pyrazolo[1,5-a]pyrimidine-3,7-dicarboxamide (51 mg) was synthesised following a similar procedure to that described in General Method E.

¹H NMR (400 MHz, DMSO-d₆) δ 10.41 (d, J=6.8 Hz, 1H), 8.93 (d, J=4.3 Hz, 1H), 7.69 (d, J=4.3 Hz, 1H), 7.65 (s, 1H), 7.60 (s, 1H), 7.35-7.27 (m, 1H), 7.07-6.96 (m, 1H), 5.88-5.78 (m, 1H), 5.03 (dd, J=10.0, 8.2 Hz, 1H), 4.99-4.87 (m, 2H), 4.69 (dd, J=10.0, 4.3 Hz, 1H), 3.30 (s, 3H)

LCMS (Method 7): 2.34 min, 404.2 [M+H]⁺

### Synthesis of Compound 120, N7-[(3R)-5-fluoro-2,3-dihydrobenzofuran-3-yl]-2-(methoxymethyl)pyrazolo[1,5-a]pyrimidine-3,7-dicarboxamide

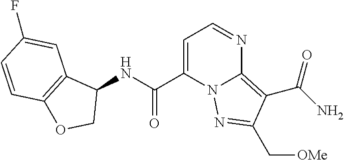 [SMILES: COCc1nn2c(C(=O)N[C@H]3COc4ccc(F)cc43)ccnc2c1C(N)=O]

N7-[(3R)-5-fluoro-2,3-dihydrobenzofuran-3-yl]-2-(methoxymethyl)pyrazolo[1,5-a]pyrimidine-3,7-dicarboxamide (34 mg) was synthesised following a similar procedure to that described in General Method E.

¹H NMR (400 MHz, DMSO-d₆) δ 10.40 (d, J=6.7 Hz, 1H), 8.93 (d, J=4.4 Hz, 1H), 7.70 (d, J=4.4 Hz, 1H), 7.65 (s, 1H), 7.60 (s, 1H), 7.36-7.29 (m, 1H), 7.12 (td, J=9.0, 2.9 Hz, 1H), 6.92 (dd, J=8.8, 4.1 Hz, 1H), 5.83-5.74 (m, 1H), 4.99-4.90 (m, 2H), 4.90-4.84 (m, 1H), 4.50 (dd, J=9.9, 4.6 Hz, 1H), 3.29 (s, 3H)

LCMS (Method 7): 2.35 min, 386.2 [M+H]⁺

### Synthesis of Compound 121, 2-(methoxymethyl)-N7-(2-methylindan-2-yl)pyrazolo[1,5-a]pyrimidine-3,7-dicarboxamide

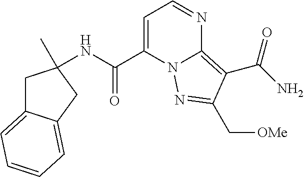 [SMILES: COCc1nn2c(C(=O)NC3(C)Cc4ccccc4C3)ccnc2c1C(N)=O]

2-(Methoxymethyl)-N7-(2-methylindan-2-yl)pyrazolo[1,5-a]pyrimidine-3,7-dicarboxamide (31 mg) was synthesised following a similar procedure to that described in General Method E.

¹H NMR (400 MHz, DMSO-d₆) δ 10.32 (s, 1H), 8.91 (d, J=4.4 Hz, 1H), 7.70 (d, J=4.4 Hz, 1H), 7.67 (s, 1H), 7.59 (s, 1H), 7.28-7.22 (m, 2H), 7.18-7.12 (m, 2H), 4.98 (s, 2H), 3.48 (d, J=16.0 Hz, 2H), 3.42 (s, 3H), 3.11 (d, J=16.0 Hz, 2H), 1.62 (s, 3H)

LCMS (Method 7): 2.66 min, 380.2 [M+H]⁺

### Synthesis of Compound 122, N7-[1-(4-fluorophenyl)ethyl]-2-(methoxymethyl)pyrazolo[1,5-a]pyrimidine-3,7-dicarboxamide

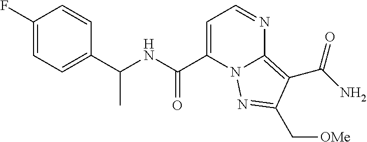 [SMILES: COCc1nn2c(C(=O)NC(C)c3ccc(F)cc3)ccnc2c1C(N)=O]

N7-[1-(4-fluorophenyl)ethyl]-2-(methoxymethyl)pyrazolo[1,5-a]pyrimidine-3,7-dicarboxamide (30 mg) was synthesised following a similar procedure to that described in General Method E.

¹H NMR (400 MHz, CDCl₃) δ 10.97 (d, J=7.4 Hz, 1H), 8.79 (d, J=4.4 Hz, 1H), 8.02 (s, 1H), 7.92 (d, J=4.4 Hz, 1H), 7.49-7.40 (m, 2H), 7.11-7.01 (m, 2H), 5.60 (s, 1H), 5.38-5.27 (m, 1H), 5.18 (d, J=2.7 Hz, 2H), 3.56 (s, 3H), 1.67 (d, J=6.9 Hz, 3H)

LCMS (Method 7): 2.48 min, 372.2 [M+H]⁺

### Synthesis of Compound 123, 2-(methoxymethyl)-N7-[(4S)-7-fluorochroman-4-yl]pyrazolo[1,5-a]pyrimidine-3,7-dicarboxamide

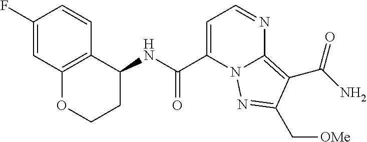 [SMILES: COCc1nn2c(C(=O)N[C@H]3CCOc4cc(F)ccc43)ccnc2c1C(N)=O]

2-(Methoxymethyl)-N7-[(4S)-7-fluorochroman-4-yl]pyrazolo[1,5-a]pyrimidine-3,7-dicarboxamide (54 mg) was synthesised following a similar procedure to that described in General Method E.

¹H NMR (400 MHz, DMSO-d₆) δ 10.26 (d, J=7.3 Hz, 1H), 8.93 (d, J=4.4 Hz, 1H), 7.70 (d, J=4.4 Hz, 1H), 7.66 (s, 1H), 7.59 (s, 1H), 7.52-7.44 (m, 1H), 6.76 (td, J=8.5, 2.7 Hz, 1H), 6.71 (dd, J=10.5, 2.6 Hz, 1H), 5.29 (d, J=6.5 Hz, 1H), 4.95 (s, 2H), 4.43-4.33 (m, 1H), 4.27 (ddd, J=11.5, 8.7, 2.9 Hz, 1H), 3.30 (s, 3H), 2.31-2.21 (m, 1H), 2.16-2.06 (m, 1H)

LCMS (Method 7): 2.47 min, 400.2 [M+H]⁺

### Synthesis of Compound 124, N7-(2,2-difluoro-6-methyl-spiro[3.3]heptan-6-yl)-2-(methoxymethyl)pyrazolo[1,5-a]pyrimidine-3,7-dicarboxamide

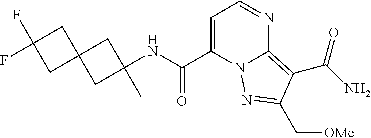 [SMILES: COCc1nn2c(C(=O)NC3(C)CC4(CC(F)(F)C4)C3)ccnc2c1C(N)=O]

N7-(2,2-difluoro-6-methyl-spiro[3.3]heptan-6-yl)-2-(methoxymethyl)pyrazolo[1,5-a]pyrimidine-3,7-dicarboxamide (31 mg) was synthesised following a similar procedure to that described in General Method E.

¹H NMR (400 MHz, DMSO-d₆) δ 10.12 (s, 1H), 8.92 (d, J=4.4 Hz, 1H), 7.69 (s, 1H) 7.66 (dd, J=4.4 Hz, 1H), 7.60 (s, 1H), 5.00 (s, 2H), 3.44 (s, 3H), 2.76-2.63 (m, 4H), 2.62-2.55 (m, 2H), 2.33 (d, J=12.6 Hz, 2H), 1.52 (s, 3H)

LCMS (Method 7): 2.55 min, 394.2 [M+H]⁺

### Synthesis of Compound 125, 2-(methoxymethyl)-N7-[2,2,2-trifluoro-1-(4-fluorophenyl)ethyl]pyrazolo[1,5-a]pyrimidine-3,7-dicarboxamide

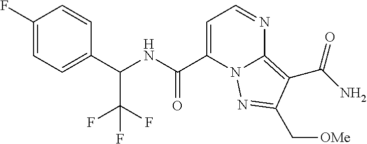 [SMILES: COCc1nn2c(C(=O)NC(c3ccc(F)cc3)C(F)(F)F)ccnc2c1C(N)=O]

2-(Methoxymethyl)-N7-[2,2,2-trifluoro-1-(4-fluorophenyl)ethyl]pyrazolo[1,5-a]pyrimidine-3,7-dicarboxamide (27 mg) was synthesised following a similar procedure to that described in General Method E.

¹H NMR (400 MHz, DMSO-d₆) δ 11.25 (d, J=9.2 Hz, 1H), 8.94 (d, J=4.4 Hz, 1H), 7.80-7.71 (m, 3H), 7.65 (d, J=11.4 Hz, 2H), 7.38-7.28 (m, 2H), 6.25 (p, J=8.2 Hz, 1H), 5.04 (s, 2H), 3.46 (s, 3H)

LCMS (Method 7): 2.75 min, 426.2 [M+H]⁺

### Synthesis of Compound 126, N7-(2,2-difluorospiro[3.3]heptan-6-yl)-2-(methoxymethyl)pyrazolo[1,5-a]pyrimidine-3,7-dicarboxamide

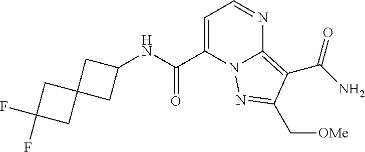 [SMILES: COCc1nn2c(C(=O)NC3CC4(C3)CC(F)(F)C4)ccnc2c1C(N)=O]

N7-(2,2-difluorospiro[3.3]heptan-6-yl)-2-(methoxymethyl)pyrazolo[1,5-a]pyrimidine-3,7-dicarboxamide (57 mg) was synthesised following a similar procedure to that described in General Method E.

¹H NMR (400 MHz, DMSO-d₆) δ 10.10 (d, J=7.1 Hz, 1H), 8.91 (d, J=4.3 Hz, 1H), 7.68 (s, 1H), 7.64 (d, J=4.3 Hz, 1H), 7.60 (s, 1H), 5.00 (s, 2H), 4.48-4.34 (m, 1H), 3.44 (s, 3H), 2.78-2.53 (m, 6H), 2.26-2.14 (m, 2H)

LCMS (Method 7): 2.39 min, 380.2 [M+H]⁺

### Synthesis of Compound 127, N7-[1-(4-fluorophenyl)cyclopropyl]-2-(methoxymethyl)pyrazolo[1,5-a]pyrimidine-3,7-dicarboxamide

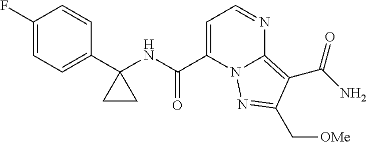 [SMILES: COCc1nn2c(C(=O)NC3(c4ccc(F)cc4)CC3)ccnc2c1C(N)=O]

N7-[1-(4-fluorophenyl)cyclopropyl]-2-(methoxymethyl)pyrazolo[1,5-a]pyrimidine-3,7-dicarboxamide (61 mg) was synthesised following a similar procedure to that described in General Method E.

¹H NMR (400 MHz, DMSO-d₆) δ 10.28 (s, 1H), 8.91 (d, J=4.3 Hz, 1H), 7.63 (d, J=35.3 Hz, 2H), 7.59 (d, J=4.4 Hz, 1H), 7.50-7.40 (m, 2H), 7.18-7.07 (m, 2H), 5.02 (s, 2H), 3.45 (s, 3H), 1.36 (s, 4H)

LCMS (Method 7): 2.44 min, 384.2 [M+H]⁺

### Synthesis of Compound 128, 2-(methoxymethyl)-N7-[(3R)-6-fluoro-2,3-dihydrobenzofuran-3-yl]pyrazolo[1,5-a]pyrimidine-3,7-dicarboxamide

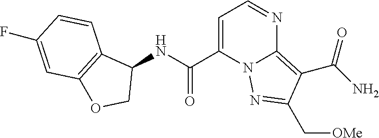 [SMILES: COCc1nn2c(C(=O)N[C@H]3COc4cc(F)ccc43)ccnc2c1C(N)=O]

2-(Methoxymethyl)-N7-[(3R)-6-fluoro-2,3-dihydrobenzofuran-3-yl]pyrazolo[1,5-a]pyrimidine-3,7-dicarboxamide (27 mg) was synthesised following a similar procedure to that described in General Method E.

¹H NMR (400 MHz, DMSO-d₆) δ 10.37 (d, J=6.7 Hz, 1H), 8.93 (d, J=4.4 Hz, 1H), 7.70 (dd, J=4.3, 0.8 Hz, 1H), 7.65 (s, 1H), 7.59 (s, 1H), 7.53-7.45 (m, 1H), 6.88-6.75 (m, 2H), 5.78-5.69 (m, 1H), 4.99-4.86 (m, 3H), 4.56 (dd, J=10.0, 4.1 Hz, 1H), 3.28 (s, 3H)

LCMS (Method 7): 2.39 min, 386.2 [M+H]⁺

### Synthesis of Compound 129, N7-(3,3-difluoroindan-1-yl)-2-(methoxymethyl)pyrazolo[1,5-a]pyrimidine-3,7-dicarboxamide

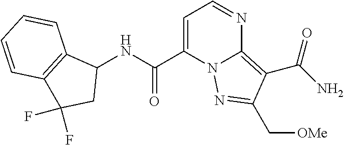 [SMILES: COCc1nn2c(C(=O)NC3CC(F)(F)c4ccccc43)ccnc2c1C(N)=O]

N7-(3,3-difluoroindan-1-yl)-2-(methoxymethyl)pyrazolo[1,5-a]pyrimidine-3,7-dicarboxamide (27 mg) was synthesised following a similar procedure to that described in General Method E.

¹H NMR (400 MHz, CDCl₃) δ 10.98 (d, J=6.9 Hz, 1H), 8.83 (d, J=4.4 Hz, 1H), 8.06-7.93 (m, 2H), 7.65 (d, J=7.5 Hz, 1H), 7.61-7.56 (m, 2H), 7.56-7.49 (m, 1H), 5.82-5.75 (m, 1H), 5.59 (s, 1H), 5.14-5.00 (m, 2H), 3.40 (s, 3H), 3.37-3.23 (m, 1H), 2.72-2.56 (m, 1H) LCMS (Method 7): 2.51 min, 402.2 [M+H]⁺

### Synthesis of Compounds 173 and 174, N7-(3,3-difluoroindan-1-yl)-2-(methoxymethyl)pyrazolo[1,5-a]pyrimidine-3,7-dicarboxamide

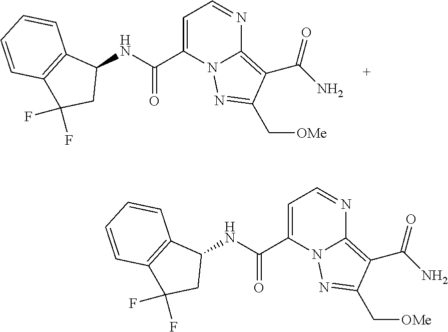 [SMILES: COCc1nn2c(C(=O)N[C@H]3CC(F)(F)c4ccccc43)ccnc2c1C(N)=O ; COCc1nn2c(C(=O)N[C@@H]3CC(F)(F)c4ccccc43)ccnc2c1C(N)=O]

Racemic N7-(3,3-difluoroindan-1-yl)-2-(methoxymethyl)pyrazolo[1,5-a]pyrimidine-3,7-dicarboxamide (39 mg) was separated by preparative SFC (eluting with 20% MeOH in CO₂) to provide enantiomer 1 (14 mg) and enantiomer 2 (15 mg) with unknown absolute configurations. Enantiomer 1 has a retention time of 4.14 minutes and enantiomer 2 has a retention time of 5.73 minutes.

¹H NMR (400 MHz, CDCl₃) δ 10.99 (d, J=7.2 Hz, 1H), 8.84 (d, J=4.4 Hz, 1H), 8.04-7.97 (m, 2H), 7.66 (d, J=7.5 Hz, 1H), 7.63-7.48 (m, 3H), 5.84-5.74 (m, 1H), 5.61 (br. s, 1H), 5.15-5.01 (m, 2H), 3.41 (s, 3H), 3.37-3.23 (m, 1H), 2.73-2.57 (m, 1H)

LCMS (Method 7): 2.42 min, 402.2 [M+H]⁺

### Synthesis of Compound 130, 2-cyclopropyl-N7-indan-2-yl-pyrazolo[1,5-a]pyrimidine-3,7-dicarboxamide

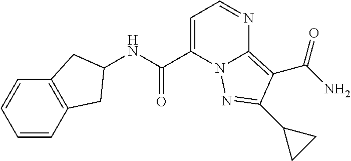 [SMILES: NC(=O)c1c(C2CC2)nn2c(C(=O)NC3Cc4ccccc4C3)ccnc12]

2-Cyclopropyl-N7-indan-2-yl-pyrazolo[1,5-a]pyrimidine-3,7-dicarboxamide (33 mg) was synthesised following a similar procedure to that described in General Method E.

¹H NMR (400 MHz, DMSO-d₆) δ 10.19 (d, J=7.4 Hz, 1H), 8.84 (d, J=4.5 Hz, 1H), 7.74 (d, J=2.8 Hz, 1H), 7.66 (d, J=4.4 Hz, 1H), 7.52 (s, 1H), 7.36-7.28 (m, 2H), 7.24-7.18 (m, 2H), 4.84-4.73 (m, 1H), 3.36 (dd, J=16.1, 6.7 Hz, 2H), 3.15-3.06 (m, 1H), 2.93 (dd, J=16.1, 3.5 Hz, 2H), 1.05-0.96 (m, 2H), 0.73-0.65 (m, 2H)

LCMS (Method 7): 2.76 min, 362.2 [M+H]⁺

### Synthesis of Compound 131, N7-(2,2-difluorospiro[3.3]heptan-7-yl)-2-(methoxymethyl)pyrazolo[1,5-a]pyrimidine-3,7-dicarboxamide

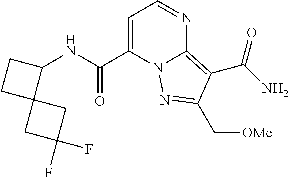 [SMILES: COCc1nn2c(C(=O)NC3CCC34CC(F)(F)C4)ccnc2c1C(N)=O]

N7-(2,2-difluorospiro[3.3]heptan-7-yl)-2-(methoxymethyl)pyrazolo[1,5-a]pyrimidine-3,7-dicarboxamide (32 mg) was synthesised following a similar procedure to that described in General Method E.

¹H NMR (400 MHz, DMSO-d₆) δ 10.10 (d, J=7.3 Hz, 1H), 8.92 (d, J=4.4 Hz, 1H), 7.68 (s, 1H), 7.65 (d, J=4.3 Hz, 1H), 7.60 (s, 1H), 5.00 (s, 2H), 4.58-4.48 (m, 1H), 3.42 (s, 3H), 3.13-2.97 (m, 1H), 2.84-2.69 (m, 1H), 2.62-2.51 (m, 2H), 2.33-2.22 (m, 1H), 2.08-1.93 (m, 2H), 1.93-1.80 (m, 1H)

LCMS (Method 7): 2.40 min, 380.2 [M+H]⁺

### Synthesis of Compound 132, 2-(methoxymethyl)-N7-[cis-4-(trifluoromethoxy)cyclohexyl]pyrazolo[1,5-a]pyrimidine-3,7-dicarboxamide

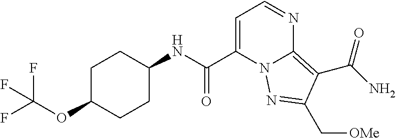 [SMILES: COCc1nn2c(C(=O)N[C@H]3CC[C@@H](OC(F)(F)F)CC3)ccnc2c1C(N)=O]

2-(Methoxymethyl)-N7-[cis-4-(trifluoromethoxy)cyclohexyl]pyrazolo[1,5-a]pyrimidine-3,7-dicarboxamide (102 mg) was synthesised following a similar procedure to that described in General Method E.

¹H NMR (400 MHz, DMSO-d₆) δ 10.23 (d, J=7.6 Hz, 1H), 8.93 (d, J=4.4 Hz, 1H), 7.74-7.65 (m, 2H), 7.61 (s, 1H), 5.02 (s, 2H), 4.62-4.52 (m, 1H), 4.15-4.05 (m, 1H), 3.43 (s, 3H), 2.00-1.69 (m, 8H)

LCMS (Method 7): 2.61 min, 416.2 [M+H]⁺

### Synthesis of Compound 133, N7-[(4,4-difluorocyclohexyl)methyl]-2-(methoxymethyl)pyrazolo[1,5-a]pyrimidine-3,7-dicarboxamide

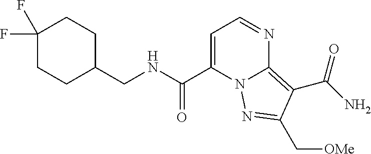 [SMILES: COCc1nn2c(C(=O)NCC3CCC(F)(F)CC3)ccnc2c1C(N)=O]

N7-[(4,4-difluorocyclohexyl)methyl]-2-(methoxymethyl)pyrazolo[1,5-a]pyrimidine-3,7-dicarboxamide (109 mg) was synthesised following a similar procedure to that described in General Method E.

¹H NMR (400 MHz, DMSO-d₆) δ 9.95 (t, J=5.8 Hz, 1H), 8.92 (d, J=4.4 Hz, 1H), 7.72-7.64 (m, 2H), 7.60 (s, 1H), 4.99 (s, 2H), 3.43-3.35 (m, 5H), 2.11-1.96 (m, 2H), 1.95-1.71 (m, 5H), 1.38-1.25 (m, 2H)

LCMS (Method 7): 2.31 min, 382.2 [M+H]⁺

### Synthesis of 2-(methoxymethyl)-N7-(1-oxoindan-2-yl)pyrazolo[1,5-a]pyrimidine-3,7-dicarboxamide

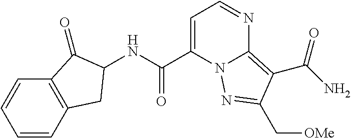 [SMILES: COCc1nn2c(C(=O)NC3Cc4ccccc4C3=O)ccnc2c1C(N)=O]

2-(Methoxymethyl)-N7-(1-oxoindan-2-yl)pyrazolo[1,5-a]pyrimidine-3,7-dicarboxamide (290 mg) was synthesised following a similar procedure to that described in General Method E.

¹H NMR (400 MHz, CDCl₃) δ 11.01 (d, J=6.4 Hz, 1H), 8.81 (d, J=4.3 Hz, 1H), 8.05 (s, 1H), 7.93 (d, J=4.4 Hz, 1H), 7.87 (d, J=7.7 Hz, 1H), 7.68 (td, J=7.5, 1.3 Hz, 1H), 7.52 (d, J=7.8, 0.9 Hz, 1H), 7.46 (t, J=7.5 Hz, 1H), 5.62 (s, 1H), 5.19 (s, 2H), 4.85-4.69 (m, 1H), 3.83 (dd, J=16.7, 8.2 Hz, 1H), 3.60 (s, 3H), 3.29 (dd, J=16.7, 5.6 Hz, 1H)

LCMS (Method 7): 1.99 min, 380.2 [M+H]⁺

### Synthesis of 2-(methoxymethyl)-N7-spiro[1,3-dithiolane-2,1′-indane]-2′-yl-pyrazolo[1,5-a]pyrimidine-3,7-dicarboxamide

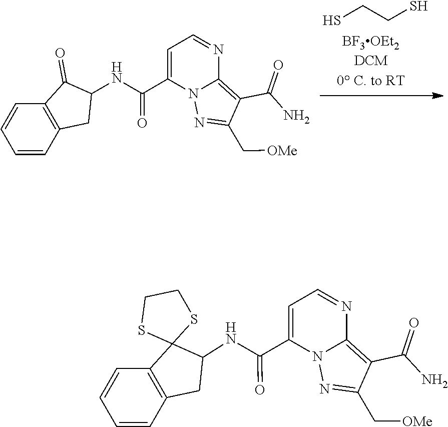

BF₃·OEt₂ (380 μL, 3.06 mmol) was added to a solution of 2-(methoxymethyl)-N7-(1-oxoindan-2-yl)pyrazolo[1,5-a]pyrimidine-3,7-dicarboxamide (290 mg, 0.76 mmol) and ethane-1,2-dithiol (96 μL, 1.15 mmol) in DCM (3.8 mL) at 0° C. The reaction was warmed to room temperature and stirred overnight. The reaction mixture was quenched with sat. aq. NaHCO₃ and diluted with EtOAc. The layers were separated, and the organic layer was dried over Na₂SO₄ and concentrated in vacuo to provide the title compound (241 mg).

¹H NMR (400 MHz, CDCl₃) δ 11.07 (d, J=8.9 Hz, 1H), 8.76 (d, J=4.4 Hz, 1H), 8.01 (s, 1H), 7.95 (d, J=4.3 Hz, 1H), 7.54 (d, J=7.8 Hz, 1H), 7.26 (t, J=7.4 Hz, 1H), 7.21 (dd, J=7.4, 1.4 Hz, 1H), 7.13 (d, J=7.4 Hz, 1H), 5.58 (s, 1H), 5.12 (td, J=8.7, 7.1 Hz, 1H), 5.03 (d, J=1.7 Hz, 2H), 3.55-3.44 (m, 1H), 3.45-3.40 (m, 3H), 3.39 (s, 3H), 3.37 (td, J=5.1, 2.1 Hz, 1H), 2.94-2.85 (m, 1H)

LCMS (Method 7): 2.63 min, 456.1 [M+H]⁺

### Synthesis of Compound 152, N7-(1,1-difluoroindan-2-yl)-2-(methoxymethyl)pyrazolo[1,5-a]pyrimidine-3,7-dicarboxamide

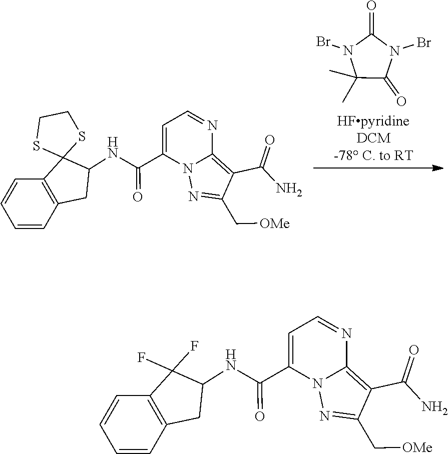 [SMILES: CC1(C)C(=O)N(Br)C(=O)N1Br ; COCc1nn2c(C(=O)NC3Cc4ccccc4C34SCCS4)ccnc2c1C(N)=O ; COCc1nn2c(C(=O)NC3Cc4ccccc4C3(F)F)ccnc2c1C(N)=O]

HF.pyridine (339 μL, 2.63 mmol) was added dropwise to a solution of 1,3-dibromo-5,5-dimethyl-imidazolidine-2,4-dione (188 mg, 0.66 mmol) in DCM (6.6 mL) at −78° C. and the mixture was stirred at −78° C. for 30 min. A solution of 2-(methoxymethyl)-N7-spiro[1,3-dithiolane-2,1′-indane]-2′-yl-pyrazolo[1,5-a]pyrimidine-3,7-dicarboxamide (200 mg, 0.44 mmol) in DCM (2.2 mL) was added dropwise, and the reaction was warmed to room temperature and stirred for 18 h. The reaction mixture was added dropwise into sat. aq. NaHCO₃ and extracted with EtOAc. The organic layer was dried over Na₂SO₄ and concentrated in vacuo. The crude product was purified by column chromatography (eluting with 0 to 2% MeOH in DCM) and preparative HPLC (basic method) to provide the title compound (20 mg).

¹H NMR (400 MHz, DMSO-d₆) δ 10.58 (d, J=8.2 Hz, 1H), 8.96 (d, J=4.4 Hz, 1H), 7.78 (d, J=4.4 Hz, 1H), 7.72-7.56 (m, 4H), 7.51-7.44 (m, 2H), 5.26-5.10 (m, 1H), 4.97 (d, J=1.0 Hz, 2H), 3.67-3.57 (m, 1H), 3.36 (s, 3H), 3.08-2.96 (m, 1H)

LCMS (Method 7): 2.43 min, 402.2 [M+H]⁺

### Synthesis of ethyl 4,6-difluorobenzofuran-3-carboxylate

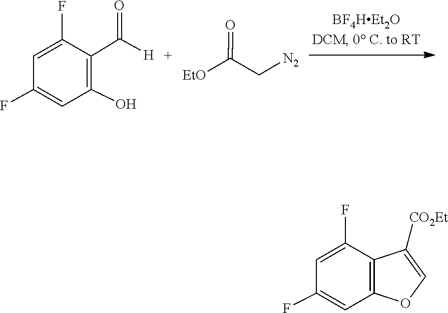

BF₄H·Et₂O (200 μL, 1.46 mmol) and ethyl diazoacetate (2.14 mL, 17.30 mmol) were added sequentially to a solution of 2,4-difluoro-6-hydroxy-benzaldehyde (2.1 g, 13.30 mmol) in DCM (20 mL) at 0° C. The reaction was warmed to room temperature, stirred for 30 min, then quenched with NaHCO₃. The mixture was diluted with DCM and water, and the layers were separated. The aqueous layer was extracted with DCM, and the combined organic layers were washed with sat. aq. K₂CO₃ and brine, dried through a hydrophobic frit, and concentrated in vacuo. The crude product was purified by column chromatography (eluting with 0 to 100% EtOAc in heptane) to provide the title compound (901 mg).

¹H NMR (400 MHz, CDCl₃) δ 8.21 (s, 1H), 7.12-7.05 (m, 1H), 6.91-6.81 (m, 1H), 4.39 (q, J=7.1 Hz, 2H), 1.40 (t, J=7.1 Hz, 3H)

LCMS (Method 3): 1.35 min, 227.2 [M+H]⁺

### Synthesis of ethyl 4,6-difluoro-2,3-dihydrobenzofuran-3-carboxylate

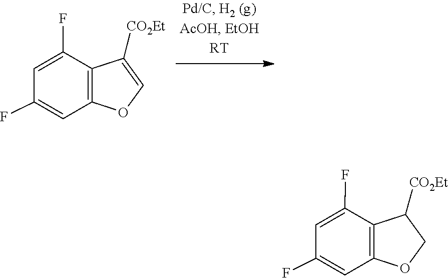 [SMILES: CCOC(=O)c1coc2cc(F)cc(F)c12 ; CCOC(=O)C1COc2cc(F)cc(F)c21]

10% Pd/C (42 mg, 0.40 mmol) was added to a solution of ethyl 4,6-difluorobenzofuran-3-carboxylate (1.8 g, 3.98 mmol) in EtOH (9 mL) and AcOH (1 mL) under argon. A balloon of hydrogen was attached, and the reaction was stirred at room temperature for 20 h. The reaction mixture was filtered through a short pad of Dicalite® and concentrated in vacuo. The residue was dissolved in DCM and washed with sat. aq. K₂CO₃. The organic layer was dried through a hydrophobic frit and concentrated in vacuo. The crude product was purified by column chromatography (eluting with 0 to 100% EtOAc in heptane) to provide the title compound (680 mg).

¹H NMR (400 MHz, CDCl₃) δ 6.43-6.35 (m, 2H), 4.91 (dd, J=9.3, 5.4 Hz, 1H), 4.80-4.69 (m, 1H), 4.37-4.32 (m, 1H), 4.25-4.20 (m, 2H), 1.30-1.26 (m, 3H)

### Synthesis of 4,6-difluoro-2,3-dihydrobenzofuran-3-carboxylic Acid

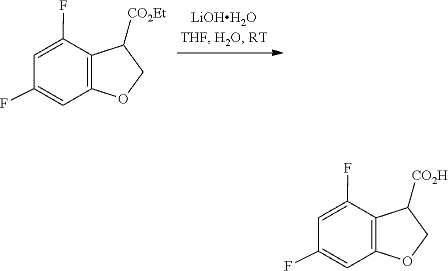 [SMILES: CCOC(=O)C1COc2cc(F)cc(F)c21 ; O=C(O)C1COc2cc(F)cc(F)c21]

A solution of LiOH·H₂O (139 mg, 3.31 mmol) in water (7.5 mL) was added to a solution of ethyl 4,6-difluoro-2,3-dihydrobenzofuran-3-carboxylate (680 mg, 3.01 mmol) in THF (7.5 mL) and the reaction was stirred at room temperature for 2 h then concentrated in vacuo. The residue was dissolved in EtOAc and water, and the layers were separated. The aqueous layer was acidified to pH 2 with 1 M aq. HCl and extracted with EtOAc. The combined organic layers were dried over Na₂SO₄ and concentrated in vacuo. The crude product was purified by column chromatography (eluting with 0 to 4% MeOH in DCM) to provide the title compound (133 mg).

¹H NMR (400 MHz, CDCl₃) δ 6.42-6.40 (m, 1H), 6.40-6.37 (m, 1H), 4.95 (dd, J=9.4, 4.9 Hz, 1H), 4.78 (dd, J=9.5, 9.5 Hz, 1H), 4.47-4.34 (m, 1H). Exchangeable proton not observed.

LCMS (Method 7): 2.10 min, 199.1 [M−H]⁻

### Synthesis of benzyl N-(4,6-difluoro-2,3-dihydrobenzofuran-3-yl)carbamate

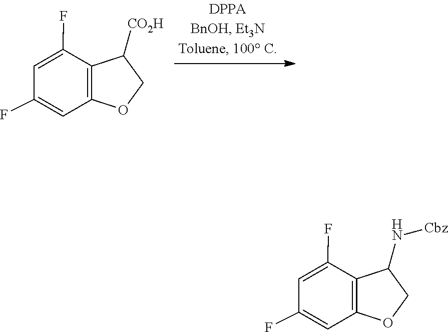 [SMILES: O=C(O)C1COc2cc(F)cc(F)c21 ; O=C(NC1COc2cc(F)cc(F)c21)OCc1ccccc1]

Et₃N (176 μL, 1.26 mmol), was added to a solution of 4,6-difluoro-2,3-dihydrobenzofuran-3-carboxylic acid (133 mg, 0.46 mmol), benzyl alcohol (78 μL, 0.75 mmol), and diphenylphosphoryl azide (163 μL, 0.76 mmol) in toluene (9 mL), and the reaction was heated at 100° C. for 1 h. The reaction mixture was cooled to room temperature and quenched with water. The layers were separated, and the organic layer was dried over Na₂SO₄ and concentrated in vacuo. The crude product was purified by column chromatography (eluting with 0 to 60% EtOAc in heptane) to provide the title compound (119 mg).

¹H NMR (400 MHz, CDCl₃) δ 7.36-7.20 (m, 5H), 6.38-6.24 (m, 2H), 5.44 (s, 1H), 5.07 (s, 2H), 4.74-4.61 (m, 1H), 4.44 (d, J=7.8 Hz, 1H). Exchangeable proton not observed.

LCMS (Method 7): 2.94 min (product does not ionise)

### Synthesis of 4,6-difluoro-2,3-dihydrobenzofuran-3-amine

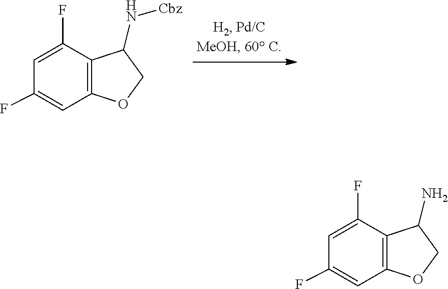 [SMILES: O=C(NC1COc2cc(F)cc(F)c21)OCc1ccccc1 ; NC1COc2cc(F)cc(F)c21]

Benzyl N-(4,6-difluoro-2,3-dihydrobenzofuran-3-yl)carbamate (119 mg, 0.39 mmol) was dissolved in MeOH (2.6 mL) and the solution was hydrogenated over 10% Pd/C at 60° C. and 1 bar (H-cube, 1 mL/min). The resultant solution was concentrated in vacuo to provide the title compound (15 mg).

¹H NMR (400 MHz, CDCl₃) δ 6.41-6.31 (m, 2H), 4.81 (dd, J=8.1, 4.0 Hz, 1H), 4.71 (dd, J=9.7, 8.1 Hz, 1H), 4.32 (dd, J=9.8, 4.0 Hz, 1H). Exchangeable protons not observed.

### Synthesis of Compound 153, N7-(4,6-difluoro-2,3-dihydrobenzofuran-3-yl)-2-(methoxymethyl)pyrazolo[1,5-a]pyrimidine-3,7-dicarboxamide

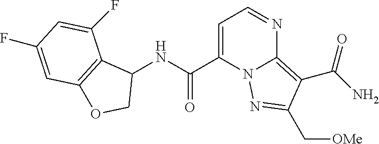 [SMILES: COCc1nn2c(C(=O)NC3COc4cc(F)cc(F)c43)ccnc2c1C(N)=O]

N7-(4,6-difluoro-2,3-dihydrobenzofuran-3-yl)-2-(methoxymethyl)pyrazolo[1,5-a]pyrimidine-3,7-dicarboxamide (11 mg) was synthesised following a similar procedure to that described in General Method E.

¹H NMR (400 MHz, DMSO-d₆) δ 10.44 (d, J=6.6 Hz, 1H), 8.92 (d, J=4.4 Hz, 1H), 7.68 (d, J=4.4 Hz, 1H), 7.65 (s, 1H), 7.59 (s, 1H), 6.88-6.75 (m, 2H), 5.95-5.84 (m, 1H), 4.98-4.89 (m, 3H), 4.66 (dd, J=10.0, 3.5 Hz, 1H), 3.29 (s, 3H)

LCMS (Method 7): 2.34 min, 404.1 [M+H]⁺

### Synthesis of 2-methyl-N-(2,4,4-trifluorocyclohexyl) propane-2-sulfinamide

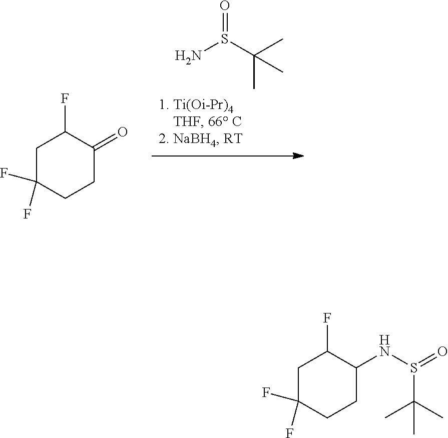

Titanium isopropoxide (290 μL, 0.99 mmol) was added to a solution of t-butylsulfinamide (96 mg, 0.79 mmol) and 2,4,4-trifluorocyclohexanone (100 mg, 0.66 mmol) in THF (5 mL) and the reaction was heated at 66° C. for 17 h then cooled to room temperature. NaBH₄ (50 mg, 1.31 mmol) was added, and the reaction was stirred at room temperature for 2 h then quenched with water. The mixture was diluted with EtOAc and the layers were separated. The organic layer was washed with water and brine, dried over Na₂SO₄, and concentrated in vacuo. The crude product was purified by column chromatography (eluting with 0 to 3% MeOH in DCM) to provide the title compound (67 mg).

LCMS (Method 6): 2.20 min, 258.2 [M+H]⁺

NMR data not reported due to a complex mixture of diastereoisomers plus fluorine splitting.

### Synthesis of 2,4,4-trifluorocyclohexanamine hydrochloride

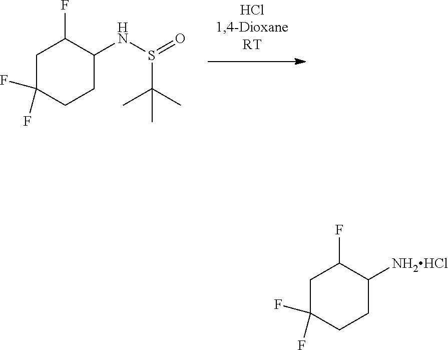

2-Methyl-N-(2,4,4-trifluorocyclohexyl) propane-2-sulfinamide (67 mg, 0.26 mmol) was dissolved in HCl (4 M in 1,4-dioxane) (2 mL, 8 mmol) and the reaction was stirred at room temperature for 16 h. The reaction mixture was concentrated in vacuo to provide the title compound (49 mg).

LCMS (Method 6): 0.16 min, 154.3 [M+H]⁺

NMR data not reported due to a complex mixture of diastereoisomers plus fluorine splitting.

### Synthesis of Compound 154, 2-(methoxymethyl)-N7-(2,4,4-trifluorocyclohexyl)pyrazolo[1,5-a]pyrimidine-3,7-dicarboxamide

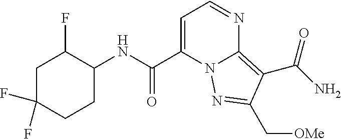 [SMILES: COCc1nn2c(C(=O)NC3CCC(F)(F)CC3F)ccnc2c1C(N)=O]

2-(Methoxymethyl)-N7-(2,4,4-trifluorocyclohexyl)pyrazolo[1,5-a]pyrimidine-3,7-dicarboxamide (21 mg, 2:1 mixture of diastereoisomers) was synthesised following a similar procedure to that described in General Method E.

¹H NMR (400 MHz, DMSO-d₆) δ 10.37 (d, J=8.1 Hz, 1H), 8.94 (d, J=4.5 Hz, 1H), 7.76 (d, J=4.3 Hz, 1H), 7.69-7.59 (m, 2H), 4.99 (d, J=1.3 Hz, 4H), 4.58-4.29 (m, 2H), 3.42 (s, 3H), 2.15-2.09 (m, 2H), 1.97-1.87 (m, 2H). Major diastereoisomer is reported.

LCMS (Method 7): 2.23 min, 386.2 [M+H]⁺

### Synthesis of 1-methylindane-1-carboxylic Acid

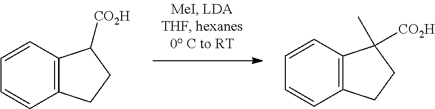

LDA (1 M in THF/hexanes) (25 mL, 24.66 mmol) was added to a solution of indane-1-carboxylic acid (2 g, 12.33 mmol) in THF (20 mL) at 0° C. and the reaction was stirred at 0° C. for 1 h then at room temperature for 1 h. MeI (2.3 mL, 36.99 mmol) was added, and the reaction was stirred at room temperature overnight. The reaction mixture was acidified with 1 M aq. HCl and diluted with DCM. The layers were separated, and the aqueous layer was extracted with DCM. The combined organic layers were washed with brine, dried over Na₂SO₄, and concentrated in vacuo to provide the title compound (2.27 g).

¹H NMR (400 MHz, DMSO-d₆) δ 12.29 (s, 1H), 7.29-7.14 (m, 4H), 3.02-2.82 (m, 2H), 2.58 (ddd, J=12.7, 8.4, 5.1 Hz, 1H), 1.93-1.81 (m, 1H), 1.43 (s, 3H)

LCMS (Method 6): 1.80 min, 175.2 [M−H]⁻

### Synthesis of 1-methylindan-1-amine

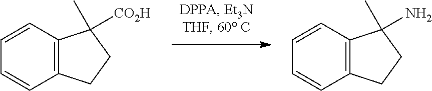

Et₃N (1.98 mL, 14.19 mmol) and diphenylphosphoryl azide (1.46 mL, 6.81 mmol) were added to a solution of 1-methylindane-1-carboxylic acid (1 g, 5.68 mmol) in THF (20 mL), and the reaction was heated at 60° C. overnight. The reaction mixture was cooled to room temperature, and then to 0° C. The mixture was acidified with 5 M aq. HCl, warmed to room temperature, and stirred overnight. The mixture was diluted with DCM, and the layers were separated. The aqueous layer was extracted with DCM, then basified with 2 M aq. NaOH and further extracted with DCM (×3). The combined organic layers were dried over Na₂SO₄ and concentrated in vacuo. The crude product was purified by SCX chromatography (eluting with 2 M NH₃ in MeOH) to provide the title compound (110 mg).

¹H NMR (400 MHz, DMSO-d₆) δ 7.33-7.26 (m, 1H), 7.21-7.07 (m, 3H), 2.85 (ddd, J=15.8, 8.4, 4.2 Hz, 1H), 2.80-2.70 (m, 1H), 1.99 (ddd, J=12.1, 7.7, 4.2 Hz, 1H), 1.89 (dt, J=12.4, 8.2 Hz, 1H), 1.26 (s, 3H). Exchangeable protons not observed.

### Synthesis of Compound 155, 2-(methoxymethyl)-N7-(1-methylindan-1-yl)pyrazolo[1,5-a]pyrimidine-3,7-dicarboxamide

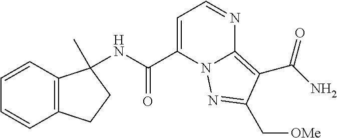 [SMILES: COCc1nn2c(C(=O)NC3(C)CCc4ccccc43)ccnc2c1C(N)=O]

### 2-(methoxymethyl)-N7-(1-methylindan-1-yl)pyrazolo[1,5-a]pyrimidine-3,7-dicarboxamide

(51 mg) was synthesised following a similar procedure to that described in General Method E.

¹H NMR (400 MHz, CDCl₃) δ 11.00 (s, 1H), 8.77 (d, J=4.4 Hz, 1H), 8.04 (s, 1H), 7.90 (d, J=4.4 Hz, 1H), 7.60-7.53 (m, 1H), 7.33-7.27 (m, 3H), 5.60 (s, 1H), 5.22-4.95 (m, 2H), 3.51 (s, 3H), 3.12 (ddd, J=15.9, 8.7, 4.9 Hz, 1H), 3.06-2.94 (m, 1H), 2.75-2.63 (m, 1H), 2.50 (ddd, J=13.1, 8.1, 4.9 Hz, 1H), 1.83 (s, 3H).

LCMS (Method 7): 2.58 min, 380.2 [M+H]⁺

### Synthesis of Compound 165, N7-(6,7-dihydro-5H-pyrrolo[1,2-a]imidazol-6-yl)-2-(methoxymethyl)pyrazolo[1,5-a]pyrimidine-3,7-dicarboxamide

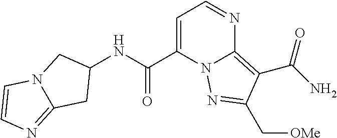 [SMILES: COCc1nn2c(C(=O)NC3Cc4nccn4C3)ccnc2c1C(N)=O]

N7-(6,7-dihydro-5H-pyrrolo[1,2-a]imidazol-6-yl)-2-(methoxymethyl)pyrazolo[1,5-a]pyrimidine-3,7-dicarboxamide (25 mg) was synthesised following a similar procedure to that described in General Method E.

¹H NMR (400 MHz, DMSO-d₆) δ 10.30 (d, J=7.1 Hz, 1H), 8.92 (d, J=4.3 Hz, 1H), 7.66 (d, J=4.4 Hz, 2H), 7.56 (s, 1H), 7.12 (d, J=1.3 Hz, 1H), 6.92 (d, J=1.3 Hz, 1H), 5.23 (tt, J=7.4, 4.1 Hz, 1H), 4.96 (s, 2H), 4.42 (dd, J=11.3, 7.2 Hz, 1H), 3.98 (dd, J=11.3, 4.1 Hz, 1H), 3.37 (s, 3H), 3.34-3.27 (m, 1H), 2.83 (dd, J=16.2, 4.3 Hz, 1H)

LCMS (Method 7): 0.87 min, 356.2 [M+H]⁺

### Synthesis of Compound 166, N7-[(4R)-7-fluorochroman-4-yl]-2-(methoxymethyl)pyrazolo[1,5-a]pyrimidine-3,7-dicarboxamide

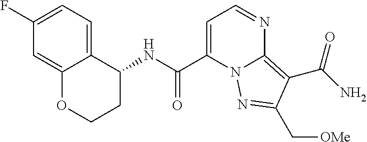 [SMILES: COCc1nn2c(C(=O)N[C@@H]3CCOc4cc(F)ccc43)ccnc2c1C(N)=O]

N7-[(4R)-7-fluorochroman-4-yl]-2-(methoxymethyl)pyrazolo[1,5-a]pyrimidine-3,7-dicarboxamide (98 mg) was synthesised following a similar procedure to that described in General Method E.

¹H NMR (400 MHz, DMSO-d₆) δ 10.27 (d, J=7.8 Hz, 1H), 8.93 (d, J=4.4 Hz, 1H), 7.70 (d, J=4.4 Hz, 1H), 7.66 (s, 1H), 7.59 (s, 1H), 7.48 (ddd, J=8.6, 6.7, 0.9 Hz, 1H), 6.81-6.67 (m, 2H), 5.34-5.25 (m, 1H), 4.95 (s, 2H), 4.38 (ddd, J=10.0, 6.3, 3.3 Hz, 1H), 4.27 (ddd, J=11.5, 8.7, 2.9 Hz, 1H), 3.30 (s, 3H), 2.31-2.20 (m, 1H), 2.17-2.05 (m, 1H)

LCMS (Method 7) 2.37 min, 400.2 [M+H]⁺

### Synthesis of Compound 167, N7-[(3S)-6-fluoro-2,3-dihydrobenzofuran-3-yl]-2-(methoxymethyl)pyrazolo[1,5-a]pyrimidine-3,7-dicarboxamide

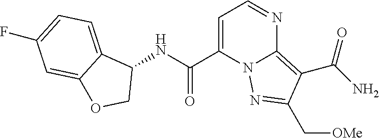 [SMILES: COCc1nn2c(C(=O)N[C@@H]3COc4cc(F)ccc43)ccnc2c1C(N)=O]

N7-[(3S)-6-fluoro-2,3-dihydrobenzofuran-3-yl]-2-(methoxymethyl)pyrazolo[1,5-a]pyrimidine-3,7-dicarboxamide (77 mg) was synthesised following a similar procedure to that described in General Method E.

¹H NMR (400 MHz, DMSO-d₆) δ 10.37 (d, J=6.7 Hz, 1H), 8.92 (d, J=4.4 Hz, 1H), 7.70 (d, J=4.4 Hz, 1H), 7.65 (s, 1H), 7.59 (s, 1H), 7.49 (ddd, J=8.3, 5.9, 0.9 Hz, 1H), 6.88-6.75 (m, 2H), 5.74 (td, J=7.4, 4.1 Hz, 1H), 4.99-4.86 (m, 3H), 4.56 (dd, J=10.0, 4.2 Hz, 1H), 3.28 (s, 3H)

LCMS (Method 7) 2.28 min, 386.2 [M+H]⁺

### Synthesis of Compound 168, N7-(1,1-dioxo-2,3-dihydrobenzothiophen-3-yl)-2-(methoxymethyl)pyrazolo[1,5-a]pyrimidine-3,7-dicarboxamide

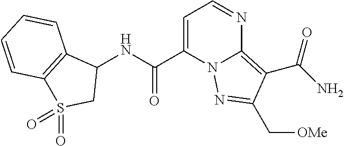 [SMILES: COCc1nn2c(C(=O)NC3CS(=O)(=O)c4ccccc43)ccnc2c1C(N)=O]

N7-(1,1-dioxo-2,3-dihydrobenzothiophen-3-yl)-2-(methoxymethyl)pyrazolo[1,5-a]pyrimidine-3,7-dicarboxamide (40 mg) was synthesised following a similar procedure to that described in General Method E.

¹H NMR (400 MHz, DMSO-d₆) δ 10.53 (s, 1H), 8.95 (d, J=4.4 Hz, 1H), 7.90-7.84 (m, 2H), 7.83-7.78 (m, 1H), 7.74-7.68 (m, 2H), 7.66 (br. s, 1H), 7.57 (br. s, 1H), 6.03-5.94 (m, 1H), 5.02-4.86 (m, 2H), 4.19 (dd, J=13.6, 7.6 Hz, 1H), 3.72 (dd, J=13.6, 5.6 Hz, 1H), 3.30 (s, 3H)

LCMS (Method 7): 1.87 min, 416.2 [M+H]⁺

### Synthesis of Compound 169, N7-[(3R)-7-chloro-2,3-dihydrobenzofuran-3-yl]-2-(methoxymethyl)pyrazolo[1,5-a]pyrimidine-3,7-dicarboxamide

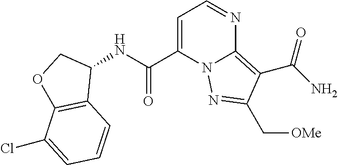 [SMILES: COCc1nn2c(C(=O)N[C@H]3COc4c(Cl)cccc43)ccnc2c1C(N)=O]

N7-[(3R)-7-chloro-2,3-dihydrobenzofuran-3-yl]-2-(methoxymethyl)pyrazolo[1,5-a]pyrimidine-3,7-dicarboxamide (20 mg) was synthesised following a similar procedure to that described in General Method E.

¹H NMR (400 MHz, DMSO-d₆) δ 10.40 (s, 1H), 8.92 (d, J=4.3 Hz, 1H), 7.70 (d, J=4.4 Hz, 1H), 7.65 (s, 1H), 7.59 (s, 1H), 7.45 (dt, J=7.5, 1.0 Hz, 1H), 7.39 (dd, J=7.9, 1.5 Hz, 1H), 6.99 (dd, J=8.0, 7.4 Hz, 1H), 5.90-5.83 (m, 1H), 4.99-4.87 (m, 3H), 4.59 (dd, J=10.0, 4.5 Hz, 1H), 3.28 (s, 3H)

LCMS (Method 7) 2.40 min; 402.2 [M+H]⁺

### Synthesis of Compound 170, N7-(6,7-dihydro-5H-cyclopenta[c]pyridin-6-yl)-2-(methoxymethyl)pyrazolo[1,5-a]pyrimidine-3,7-dicarboxamide

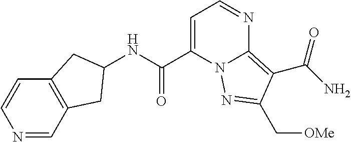 [SMILES: COCc1nn2c(C(=O)NC3Cc4ccncc4C3)ccnc2c1C(N)=O]

N7-(6,7-dihydro-5H-cyclopenta[c]pyridin-6-yl)-2-(methoxymethyl)pyrazolo[1,5-a]pyrimidine-3,7-dicarboxamide (11 mg) was synthesised following a similar procedure to that described in General Method E.

¹H NMR (400 MHz, DMSO-d₆) δ 10.18 (s, 1H), 8.91 (d, J=4.4 Hz, 1H), 8.50 (d, J=1.0 Hz, 1H), 8.38 (d, J=4.9 Hz, 1H), 7.70-7.63 (m, 2H), 7.60-7.55 (m, 1H), 7.35 (dd, J=5.0, 1.0 Hz, 1H), 4.91 (s, 2H), 4.88-4.79 (m, 1H), 3.47-3.39 (m, 2H), 3.30 (s, 3H), 3.06-2.96 (m, 2H)

LCMS (Method 7): 1.10 min, 367.2 [M+H]⁺

### Synthesis of Compound 171, N7-(3,4-dihydro-2H-pyrano[2,3-b]pyridin-4-yl)-2-(methoxymethyl)pyrazolo[1,5-a]pyrimidine-3,7-dicarboxamide

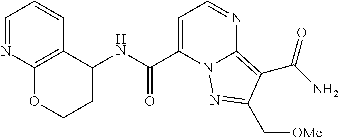 [SMILES: COCc1nn2c(C(=O)NC3CCOc4ncccc43)ccnc2c1C(N)=O]

N7-(3,4-dihydro-2H-pyrano[2,3-b]pyridin-4-yl)-2-(methoxymethyl)pyrazolo[1,5-a]pyrimidine-3,7-dicarboxamide (20 mg) was synthesised following a similar procedure to that described in General Method E.

¹H NMR (400 MHz, DMSO-d₆) δ10.23 (d, J=7.8 Hz, 1H), 8.93 (d, J=4.4 Hz, 1H), 8.13 (ddd, J=4.8, 2.0, 0.6 Hz, 1H), 7.91 (ddd, J=7.5, 2.0, 0.9 Hz, 1H), 7.69 (d, J=4.4 Hz, 1H), 7.66 (s, 1H), 7.59 (s, 1H), 7.01 (dd, J=7.5, 4.8 Hz, 1H), 5.43-5.34 (m, 1H), 4.95 (s, 2H), 4.52-4.35 (m, 2H), 3.32 (s, 3H), 2.34-2.24 (m, 1H), 2.14-2.05 (m, 1H)

LCMS (Method 7): 1.57 min, 383.2 [M+H]⁺

### Synthesis of Compound 172, N7-[(4S)-chroman-4-yl]-2-(methoxymethyl)pyrazolo[1,5-a]pyrimidine-3,7-dicarboxamide

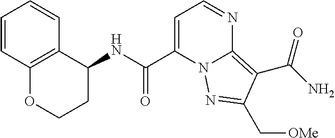 [SMILES: COCc1nn2c(C(=O)N[C@H]3CCOc4ccccc43)ccnc2c1C(N)=O]

N7-[(4S)-chroman-4-yl]-2-(methoxymethyl)pyrazolo[1,5-a]pyrimidine-3,7-dicarboxamide (31 mg) was synthesised following a similar procedure to that described in General Method E.

¹H NMR (400 MHz, DMSO-d₆) δ 10.29 (d, J=7.8 Hz, 1H), 8.93 (d, J=4.4 Hz, 1H), 7.71 (d, J=4.4 Hz, 1H), 7.66 (br. s, J=2.7 Hz, 1H), 7.59 (br. s, 1H), 7.45-7.40 (m, 1H), 7.23-7.17 (m, 1H), 6.91 (td, J=7.5, 1.2 Hz, 1H), 6.83 (dd, J=8.2, 1.2 Hz, 1H), 5.36-5.27 (m, 1H), 4.94 (s, 2H), 4.38-4.31 (m, 1H), 4.29-4.20 (m, 1H), 3.28 (s, 3H), 2.34-2.22 (m, 1H), 2.15-2.06 (m, 1H)

LCMS (Method 7): 2.29 min, 382.2 [M+H]⁺

### Synthesis of Compound 177, N7-(1,1-dioxo-2,3-dihydrobenzothiophen-3-yl)-2-(methoxymethyl)pyrazolo[1,5-a]pyrimidine-3,7-dicarboxamide

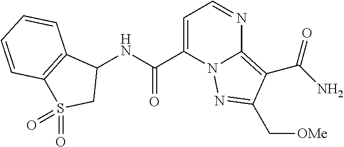 [SMILES: COCc1nn2c(C(=O)NC3CS(=O)(=O)c4ccccc43)ccnc2c1C(N)=O]

N7-(1,1-dioxo-2,3-dihydrobenzothiophen-3-yl)-2-(methoxymethyl)pyrazolo[1,5-a]pyrimidine-3,7-dicarboxamide (40 mg) was synthesised following a similar procedure to that described in General Method E.

¹H NMR (400 MHz, DMSO-d₆) δ 10.53 (s, 1H), 8.95 (d, J=4.4 Hz, 1H), 7.90-7.84 (m, 2H), 7.83-7.78 (m, 1H), 7.74-7.68 (m, 2H), 7.66 (br. s, 1H), 7.57 (br. s, 1H), 6.03-5.94 (m, 1H), 5.02-4.86 (m, 2H), 4.19 (dd, J=13.6, 7.6 Hz, 1H), 3.72 (dd, J=13.6, 5.6 Hz, 1H), 3.30 (s, 3H)

LCMS (Method 7): 1.87 min, 416.2 [M+H]⁺

### Synthesis of Compound 178, N7-[(3R)-7-chloro-2,3-dihydrobenzofuran-3-yl]-2-(methoxymethyl)pyrazolo[1,5-a]pyrimidine-3,7-dicarboxamide

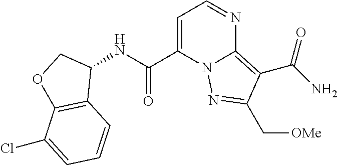 [SMILES: COCc1nn2c(C(=O)N[C@H]3COc4c(Cl)cccc43)ccnc2c1C(N)=O]

N7-[(3R)-7-chloro-2,3-dihydrobenzofuran-3-yl]-2-(methoxymethyl)pyrazolo[1,5-a]pyrimidine-3,7-dicarboxamide (20 mg) was synthesised following a similar procedure to that described in General Method E.

¹H NMR (400 MHz, DMSO-d₆) δ 10.40 (s, 1H), 8.92 (d, J=4.3 Hz, 1H), 7.70 (d, J=4.4 Hz, 1H), 7.65 (s, 1H), 7.59 (s, 1H), 7.45 (dt, J=7.5, 1.0 Hz, 1H), 7.39 (dd, J=7.9, 1.5 Hz, 1H), 6.99 (dd, J=8.0, 7.4 Hz, 1H), 5.90-5.83 (m, 1H), 4.99-4.87 (m, 3H), 4.59 (dd, J=10.0, 4.5 Hz, 1H), 3.28 (s, 3H)

LCMS (Method 7) 2.40 min; 402.2 [M+H]⁺

### Synthesis of Compound 179, N7-(6,7-dihydro-5H-cyclopenta[c]pyridin-6-yl)-2-(methoxymethyl)pyrazolo[1,5-a]pyrimidine-3,7-dicarboxamide

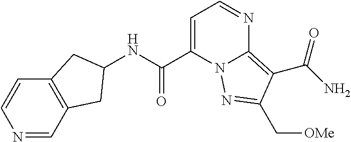 [SMILES: COCc1nn2c(C(=O)NC3Cc4ccncc4C3)ccnc2c1C(N)=O]

N7-(6,7-dihydro-5H-cyclopenta[c]pyridin-6-yl)-2-(methoxymethyl)pyrazolo[1,5-a]pyrimidine-3,7-dicarboxamide (11 mg) was synthesised following a similar procedure to that described in General Method E.

¹H NMR (400 MHz, DMSO-d₆) δ 10.18 (s, 1H), 8.91 (d, J=4.4 Hz, 1H), 8.50 (d, J=1.0 Hz, 1H), 8.38 (d, J=4.9 Hz, 1H), 7.70-7.63 (m, 2H), 7.60-7.55 (m, 1H), 7.35 (dd, J=5.0, 1.0 Hz, 1H), 4.91 (s, 2H), 4.88-4.79 (m, 1H), 3.47-3.39 (m, 2H), 3.30 (s, 3H), 3.06-2.96 (m, 2H)

LCMS (Method 7): 1.10 min, 367.2 [M+H]⁺

### Synthesis of Compound 180, N7-(3,4-dihydro-2H-pyrano[2,3-b]pyridin-4-yl)-2-(methoxymethyl)pyrazolo[1,5-a]pyrimidine-3,7-dicarboxamide

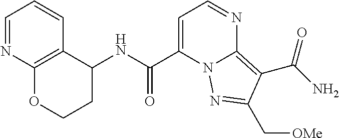 [SMILES: COCc1nn2c(C(=O)NC3CCOc4ncccc43)ccnc2c1C(N)=O]

N7-(3,4-dihydro-2H-pyrano[2,3-b]pyridin-4-yl)-2-(methoxymethyl)pyrazolo[1,5-a]pyrimidine-3,7-dicarboxamide (20 mg) was synthesised following a similar procedure to that described in General Method E.

¹H NMR (400 MHz, DMSO-d₆) δ 10.23 (d, J=7.8 Hz, 1H), 8.93 (d, J=4.4 Hz, 1H), 8.13 (ddd, J=4.8, 2.0, 0.6 Hz, 1H), 7.91 (ddd, J=7.5, 2.0, 0.9 Hz, 1H), 7.69 (d, J=4.4 Hz, 1H), 7.66 (s, 1H), 7.59 (s, 1H), 7.01 (dd, J=7.5, 4.8 Hz, 1H), 5.43-5.34 (m, 1H), 4.95 (s, 2H), 4.52-4.35 (m, 2H), 3.32 (s, 3H), 2.34-2.24 (m, 1H), 2.14-2.05 (m, 1H)

LCMS (Method 7): 1.57 min, 383.2 [M+H]⁺

### Synthesis of Compound 181, N7-[(4S)-chroman-4-yl]-2-(methoxymethyl)pyrazolo[1,5-a]pyrimidine-3,7-dicarboxamide

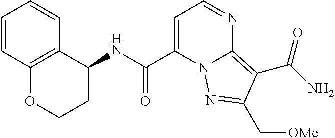 [SMILES: COCc1nn2c(C(=O)N[C@H]3CCOc4ccccc43)ccnc2c1C(N)=O]

N7-[(4S)-chroman-4-yl]-2-(methoxymethyl)pyrazolo[1,5-a]pyrimidine-3,7-dicarboxamide (31 mg) was synthesised following a similar procedure to that described in General Method E.

¹H NMR (400 MHz, DMSO-d₆) δ 10.29 (d, J=7.8 Hz, 1H), 8.93 (d, J=4.4 Hz, 1H), 7.71 (d, J=4.4 Hz, 1H), 7.66 (br. s, J=2.7 Hz, 1H), 7.59 (br. s, 1H), 7.45-7.40 (m, 1H), 7.23-7.17 (m, 1H), 6.91 (td, J=7.5, 1.2 Hz, 1H), 6.83 (dd, J=8.2, 1.2 Hz, 1H), 5.36-5.27 (m, 1H), 4.94 (s, 2H), 4.38-4.31 (m, 1H), 4.29-4.20 (m, 1H), 3.28 (s, 3H), 2.34-2.22 (m, 1H), 2.15-2.06 (m, 1H)

LCMS (Method 7): 2.29 min, 382.2 [M+H]⁺

### Synthesis of 6,7-difluorochroman-4-one oxime

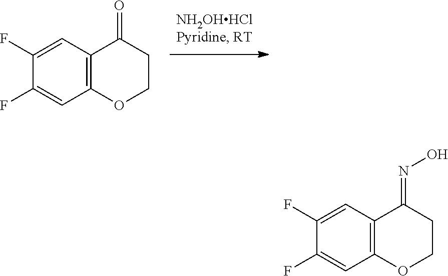 [SMILES: O=C1CCOc2cc(F)c(F)cc21 ; O/N=C1\CCOc2cc(F)c(F)cc21]

NH₂OH·HCl (377 mg, 5.43 mmol) was added to a solution of 6,7-difluorochroman-4-one (500 mg, 2.72 mmol) in pyridine (5 mL) and the reaction was stirred at room temperature for 24 h. The reaction mixture was concentrated in vacuo, and the residue was dissolved in EtOAc and water. The layers were separated, and the aqueous layer was extracted with EtOAc (×2). The combined organic layers were dried through a hydrophobic frit and concentrated in vacuo to provide the title compound (532 mg).

¹H NMR (400 MHz, CDCl₃) δ 7.61 (dd, J=11.1, 9.0 Hz, 1H), 7.56 (s, 1H), 6.71 (dd, J=11.1, 6.9 Hz, 1H), 4.23 (dd, J=6.5, 5.9 Hz, 2H), 2.95 (t, J=6.3 Hz, 2H)

LCMS (Method 6): 1.84 min, 200.2 [M+H]⁺

### Synthesis of 6,7-difluorochroman-4-amine

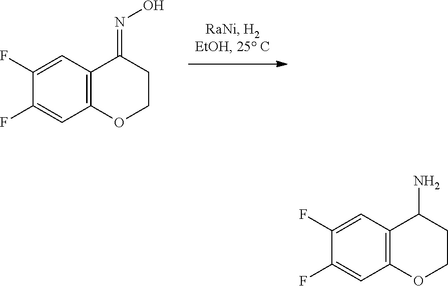 [SMILES: O/N=C1\CCOc2cc(F)c(F)cc21 ; NC1CCOc2cc(F)c(F)cc21]

6,7-Difluorochroman-4-one oxime (532 mg, 2.67 mmol) was dissolved in EtOH (55 mL) and the solution was hydrogenated over RaNi at 25° C. and 10 bar (H-cube, 1 mL/min). The resultant solution was concentrated in vacuo to provide the title compound (356 mg).

¹H NMR (400 MHz, DMSO-d₆) δ 7.42 (ddd, J=11.7, 9.4, 0.8 Hz, 1H), 6.79 (dd, J=12.2, 7.1 Hz, 1H), 4.29-4.18 (m, 1H), 4.17-4.07 (m, 1H), 3.82 (t, J=5.9 Hz, 1H), 2.04 (s, 2H), 2.02-1.93 (m, 1H), 1.76-1.64 (m, 1H)

### Synthesis of Compound 182, N7-(6,7-difluorochroman-4-yl)-2-(methoxymethyl)pyrazolo[1,5-a]pyrimidine-3,7-dicarboxamide

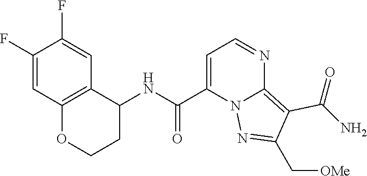 [SMILES: COCc1nn2c(C(=O)NC3CCOc4cc(F)c(F)cc43)ccnc2c1C(N)=O]

N7-(6,7-difluorochroman-4-yl)-2-(methoxymethyl)pyrazolo[1,5-a]pyrimidine-3,7-dicarboxamide (72 mg) was synthesised following a similar procedure to that described in General Method E.

¹H NMR (400 MHz, DMSO-d₆) δ 10.25 (d, J=7.8 Hz, 1H), 8.93 (d, J=4.3 Hz, 1H), 7.70 (d, J=4.4 Hz, 1H), 7.66 (s, 1H), 7.59 (s, 1H), 7.51 (dd, J=11.3, 9.1 Hz, 1H), 6.97 (dd, J=12.0, 7.1 Hz, 1H), 5.34-5.24 (m, 1H), 5.00-4.89 (m, 2H), 4.40-4.31 (m, 1H), 4.31-4.20 (m, 1H), 3.31 (s, 3H), 2.31-2.20 (m, 1H), 2.14-2.04 (m, 1H)

LCMS (Method 7): 2.45 min, 418.2 [M+H]⁺

### Synthesis of Compound 183, 2-(methoxymethyl)-N7-[(1S)-indan-1-yl]pyrazolo[1,5-a]pyrimidine-3,7-dicarboxamide

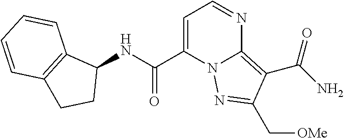 [SMILES: COCc1nn2c(C(=O)N[C@H]3CCc4ccccc43)ccnc2c1C(N)=O]

2-(Methoxymethyl)-N7-[(1S)-indan-1-yl]pyrazolo[1,5-a]pyrimidine-3,7-dicarboxamide (65 mg) was synthesised following a similar procedure to that described in General Method E.

¹H NMR (400 MHz, DMSO-d₆) δ 10.23 (d, J=7.7 Hz, 1H), 8.93 (d, J=4.4 Hz, 1H), 7.72 (d, J=4.4 Hz, 1H), 7.70-7.64 (m, 1H), 7.59 (s, 1H), 7.48 (d, J=7.1 Hz, 1H), 7.35-7.20 (m, 3H), 5.61-5.51 (m, 1H), 4.95 (d, J=1.6 Hz, 2H), 3.32 (s, 3H), 3.08-2.86 (m, 2H), 2.68-2.56 (m, 1H), 2.03-1.89 (m, 1H)

LCMS (Method 7): 2.41 min, 366.2 [M+H]⁺

### Synthesis of methyl 3-hydroxyfuro[2,3-b]pyridine-2-carboxylate

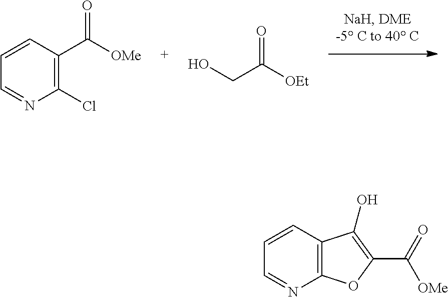 [SMILES: COC(=O)c1cccnc1Cl ; CCOC(=O)CO ; COC(=O)c1oc2ncccc2c1O]

NaH (1.87 g, 46.63 mmol) was added to a solution of ethyl 2-hydroxyacetate (2.65 mL, 27.98 mmol) in DME (50 mL) at −5° C. The mixture was warmed to room temperature and stirred for 30 mins. A solution of methyl 2-chloropyridine-3-carboxylate (4 g, 23.31 mmol) in DME (15 mL) was added slowly and the reaction was heated at 40° C. overnight. The resulting solid was dissolved in sat. aq. NaHCO₃ and the mixture was diluted with EtOAc. The layers were separated, and the aqueous layer was acidified to pH 4 with AcOH and extracted with DCM. The combined organic layers were dried through a hydrophobic frit and concentrated in vacuo. The residue was triturated with heptane, and the resulting precipitate was collected by filtration and dried to provide the title compound (1.99 g).

¹H NMR (400 MHz, DMSO-d₆) δ 8.50 (dd, J=4.8, 1.7 Hz, 1H), 8.37 (m, 1H), 7.42 (m, 1H), 3.84 (s, 3H). Exchangeable proton not observed.

LCMS (Method 6): 1.38 min, 194.2 [M+H]⁺

### Synthesis of furo[2,3-b]pyridin-3-one

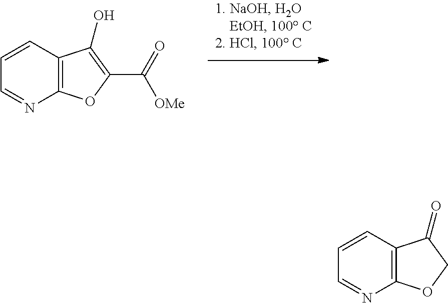 [SMILES: COC(=O)c1oc2ncccc2c1O ; O=C1COc2ncccc21]

A solution of NaOH (1.25 g, 31.17 mmol) in water (4 mL) was added to a solution of methyl 3-hydroxyfuro[2,3-b]pyridine-2-carboxylate (1.5 g, 7.77 mmol) in EtOH (25 mL) and the reaction was heated at 100° C. for 1 h. The reaction mixture was cooled to room temperature and concentrated in vacuo. The residue was dissolved in water and the solution was acidified to pH 2-3 with concentrated HCl. The reaction was heated at 100° C. for 2 h then cooled to room temperature. The mixture was neutralised with NaHCO₃ and diluted with DCM. The layers were separated, and the organic layer was dried through a hydrophobic frit and concentrated in vacuo to provide the title compound (960 mg).

¹H NMR (400 MHz, DMSO-d₆) δ 8.60 (dd, J=4.9, 1.9 Hz, 1H), 8.15 (dd, J=7.5, 1.9 Hz, 1H), 7.26 (dd, J=7.5, 4.9 Hz, 1H), 4.89 (s, 2H)

LCMS (Method 6): 0.69 min, 136.2 [M+H]⁺

### Synthesis of furo[2,3-b]pyridin-3-one oxime

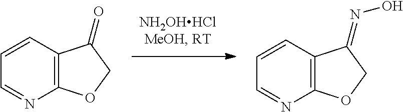 [SMILES: O=C1COc2ncccc21 ; O/N=C1\COc2ncccc21]

Hydroxylamine hydrochloride (551 mg, 7.93 mmol) was added to a solution of furo[2,3-b]pyridin-3-one (357 mg, 2.64 mmol) in MeOH (5 mL) and the reaction was stirred at room temperature overnight. The reaction mixture was concentrated in vacuo and diluted with EtOAc and water. The resulting precipitate was collected by filtration and dried to provide the title compound (128 mg).

¹H NMR (400 MHz, DMSO-d₆) δ 11.60 (s, 1H), 8.20 (dd, J=5.1, 1.8 Hz, 1H), 7.96 (dd, J=7.4, 1.8 Hz, 1H), 7.07 (dd, J=7.4, 5.0 Hz, 1H), 5.18 (s, 2H)

LCMS (Method 6): 1.00 min, 151.2 [M+H]⁺

### Synthesis of 2,3-dihydrofuro[2,3-b]pyridin-3-amine

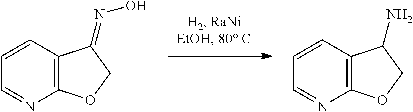 [SMILES: O/N=C1\COc2ncccc21 ; NC1COc2ncccc21]

Furo[2,3-b]pyridin-3-one oxime (150 mg, 1.00 mmol) was dissolved in EtOH (50 mL) and the solution was hydrogenated over RaNi at 80° C. and 70 bar (H-cube, 1 mL/min). The resultant solution was concentrated in vacuo to provide the title compound (141 mg). The crude product was taken forward to the next step without purification.

### Synthesis of Compound 184, N7-(2,3-dihydrofuro[2,3-b]pyridin-3-yl)-2-(methoxymethyl)pyrazolo[1,5-a]pyrimidine-3,7-dicarboxamide

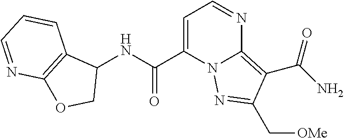 [SMILES: COCc1nn2c(C(=O)NC3COc4ncccc43)ccnc2c1C(N)=O]

N7-(2,3-dihydrofuro[2,3-b]pyridin-3-yl)-2-(methoxymethyl)pyrazolo[1,5-a]pyrimidine-3,7-dicarboxamide (49 mg) was synthesised following a similar procedure to that described in General Method E.

¹H NMR (400 MHz, DMSO-d₆) δ 10.40 (s, 1H), 8.93 (d, J=4.4 Hz, 1H), 8.12 (ddd, J=5.1, 1.8, 0.7 Hz, 1H), 7.90 (ddd, J=7.3, 1.8, 1.0 Hz, 1H), 7.71-7.56 (m, 3H), 7.01 (dd, J=7.3, 5.1 Hz, 1H), 5.83 (dd, J=8.6, 4.5 Hz, 1H), 4.96-4.86 (m, 3H), 4.50 (dd, J=10.0, 4.5 Hz, 1H), 3.30 (s, 3H)

LCMS (Method 7): 1.63 min, 369.2 [M+H]⁺

### Synthesis of 5,7-difluoro-2H-chromene-3-carbonitrile

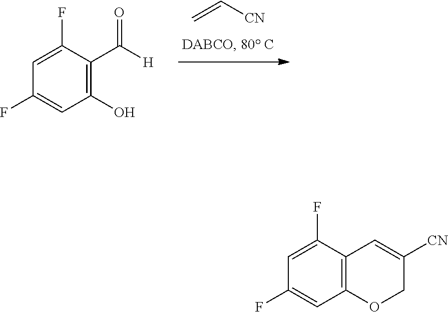

DABCO (1.7 g, 15.81 mmol) was added to a solution of 2,4-difluoro-6-hydroxy-benzaldehyde (2.5 g, 15.81 mmol) in prop-2-enenitrile (17 mL, 253.00 mmol) and the reaction was heated at 80° C. for 18 h. The reaction mixture was cooled to room temperature, quenched with 1 M aq. HCl, and diluted with EtOAc. The layers were separated, and the organic layer was washed with water and brine, dried over Na₂SO₄, and concentrated in vacuo. The crude product was purified by column chromatography (eluting with 0 to 40% EtOAc in heptane) to provide the title compound (1.46 g).

¹H NMR (400 MHz, CDCl₃) δ 7.37-7.30 (m, 1H), 6.52-6.41 (m, 2H), 4.84 (d, J=0.8 Hz, 2H)

LCMS (Method 7): 2.71 min (product does not ionise)

### Synthesis of 5,7-difluoro-2H-chromene-3-carboxylic Acid

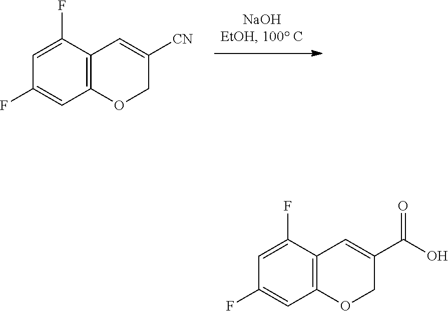 [SMILES: N#CC1=Cc2c(F)cc(F)cc2OC1 ; O=C(O)C1=Cc2c(F)cc(F)cc2OC1]

2 M aq. NaOH (24 mL, 59.26 mmol) was added to a solution of 5,7-difluoro-2H-chromene-3-carbonitrile (1.46 g, 7.41 mmol) in EtOH (9 mL) and the reaction was heated at 100° C. for 1 h. The reaction mixture was cooled to room temperature and acidified to pH 4 with 1 M aq. HCl. The resulting precipitate was collected by filtration, washed with water, and dried to provide the title compound (1.06 g).

¹H NMR (400 MHz, DMSO-d₆) δ 7.41-7.34 (m, 1H), 6.90 (td, J=9.8, 2.4 Hz, 1H), 6.75-6.67 (m, 1H), 4.98 (d, J=1.5 Hz, 2H). Exchangeable proton not observed.

LCMS (Method 7): 2.44 min, 211.1 [M−H]⁻

### Synthesis of 5,7-difluorochromane-3-carboxylic Acid

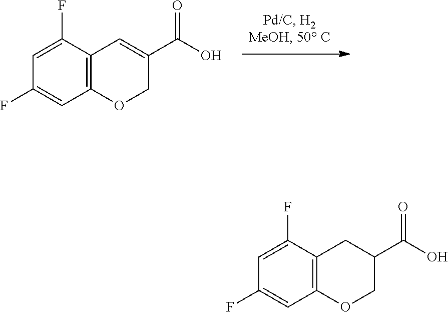 [SMILES: O=C(O)C1=Cc2c(F)cc(F)cc2OC1 ; O=C(O)C1COc2cc(F)cc(F)c2C1]

5,7-difluoro-2H-chromene-3-carboxylic acid (1.06 g, 4.71 mmol) was dissolved in MeOH (157 mL) and the solution was hydrogenated over 10% Pd/C at 50° C. (H-cube, 1 mL/min, 10 bar). The resultant solution was concentrated in vacuo to provide the title compound (797 mg).

¹H NMR (400 MHz, MeOD) δ 6.51-6.43 (m, 1H), 6.42-6.35 (m, 1H), 4.49-4.30 (m, 1H), 4.24 (dd, J=10.9, 7.5 Hz, 1H), 3.12-2.97 (m, 2H), 2.99-2.82 (m, 2H)

LCMS (Method 7): 2.35 min, 213.2 [M−H]⁻

### Synthesis of 5,7-difluorochroman-3-amine

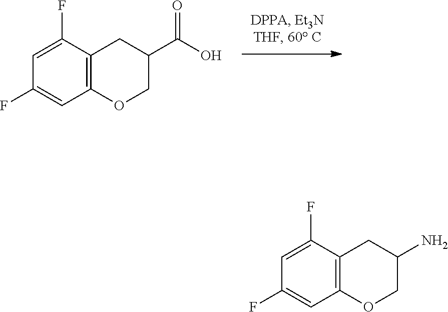 [SMILES: O=C(O)C1COc2cc(F)cc(F)c2C1 ; NC1COc2cc(F)cc(F)c2C1]

Et₃N (651 μL, 4.67 mmol) and diphenylphosphoryl azide (482 μL, 2.24 mmol) were added sequentially to a solution of 5,7-difluorochromane-3-carboxylic acid (400 mg, 1.87 mmol) in THF (4.7 mL), and the reaction was heated at 60° C. for 2 h. The reaction mixture was cooled to room temperature and then to 0° C. The solution was acidified with 5 M aq. HCl, then warmed to room temperature and stirred for 2 h. The mixture was diluted with DCM and the layers were separated. The aqueous layer was basified with 2 M aq. NaOH and extracted with DCM. The combined organic layers were dried through a hydrophobic frit and concentrated in vacuo. The crude product was purified by column chromatography (eluting with 0 to 7% MeOH in DCM) to provide the title compound as the impure product (29 mg).

LCMS (Method 6): 2.09 min, 186.2 [M+H]⁺

### Synthesis of Compound 185, N7-(5,7-difluorochroman-3-yl)-2-(methoxymethyl)pyrazolo[1,5-a]pyrimidine-3,7-dicarboxamide

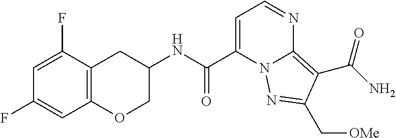 [SMILES: COCc1nn2c(C(=O)NC3COc4cc(F)cc(F)c4C3)ccnc2c1C(N)=O]

N7-(5,7-difluorochroman-3-yl)-2-(methoxymethyl)pyrazolo[1,5-a]pyrimidine-3,7-dicarboxamide (3.1 mg) was synthesised following a similar procedure to that described in General Method E.

¹H NMR (400 MHz, DMSO-d₆) δ 10.29 (d, J=7.3 Hz, 1H), 8.92 (d, J=4.4 Hz, 1H), 7.75 (d, J=4.4 Hz, 1H), 7.67 (s, 1H), 7.58 (s, 1H), 6.87-6.77 (m, 1H), 6.71-6.61 (m, 1H), 4.89-4.77 (m, 2H), 4.70-4.58 (m, 1H), 4.37-4.24 (m, 2H), 3.28 (s, 3H), 3.15-3.06 (m, 1H), 2.91-2.77 (m, 1H)

LCMS (Method 7): 2.36 min, 418.2 [M+H]⁺

### Synthesis of Compound 186, N7-(4,4-dimethyltetralin-1-yl)-2-(methoxymethyl)pyrazolo[1,5-a]pyrimidine-3,7-dicarboxamide

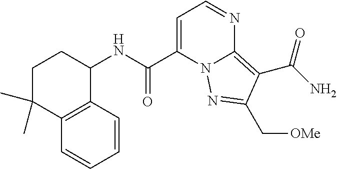 [SMILES: COCc1nn2c(C(=O)NC3CCC(C)(C)c4ccccc43)ccnc2c1C(N)=O]

N7-(4,4-dimethyltetralin-1-yl)-2-(methoxymethyl)pyrazolo[1,5-a]pyrimidine-3,7-dicarboxamide (16 mg) was synthesised following a similar procedure to that described in General Method E.

¹H NMR (400 MHz, DMSO-d₆) δ 10.33 (d, J=8.3 Hz, 1H), 8.93 (d, J=4.4 Hz, 1H), 7.74 (d, J=4.4 Hz, 1H), 7.67 (s, 1H), 7.58 (s, 1H), 7.47-7.37 (m, 2H), 7.26 (td, J=7.6, 1.5 Hz, 1H), 7.15 (td, J=7.5, 1.3 Hz, 1H), 5.32-5.23 (m, 1H), 4.99-4.87 (m, 2H), 3.25 (s, 3H), 2.19-2.08 (m, 1H), 1.98-1.83 (m, 2H), 1.78-1.67 (m, 1H), 1.34 (s, 3H), 1.28 (s, 3H)

LCMS (Method 7) 2.81 min; 408.2 [M+H]⁺

### Synthesis of Compound 187, 2-(methoxymethyl)-N7-(5,6,7,8-tetrahydroimidazo[1,2-a]pyridin-6-yl)pyrazolo[1,5-a]pyrimidine-3,7-dicarboxamide

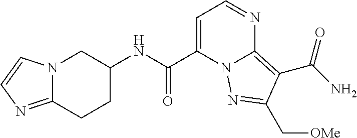 [SMILES: COCc1nn2c(C(=O)NC3CCc4nccn4C3)ccnc2c1C(N)=O]

2-(Methoxymethyl)-N7-(5,6,7,8-tetrahydroimidazo[1,2-a]pyridin-6-yl)pyrazolo[1,5-a]pyrimidine-3,7-dicarboxamide (30 mg) was synthesised following a similar procedure to that described in General Method E.

¹H NMR (400 MHz, DMSO-d₆) δ 10.31 (d, J=7.2 Hz, 1H), 8.92 (d, J=4.4 Hz, 1H), 7.71 (d, J=4.4 Hz, 1H), 7.69-7.63 (m, 1H), 7.63-7.56 (m, 1H), 7.02 (d, J=1.3 Hz, 1H), 6.84 (d, J=1.2 Hz, 1H), 4.92 (s, 2H), 4.66-4.57 (m, 1H), 4.29 (dd, J=12.8, 4.4 Hz, 1H), 4.07 (dd, J=12.9, 4.5 Hz, 1H), 3.35 (s, 3H), 3.06-2.88 (m, 2H), 2.21-2.13 (m, 2H)

LCMS (Method 7): 0.94 min, 370.2 [M+H]⁺

### Synthesis of Compound 188, 2-(methoxymethyl)-N7-(5,6,7,8-tetrahydroimidazo[1,5-a]pyridin-6-yl)pyrazolo[1,5-a]pyrimidine-3,7-dicarboxamide

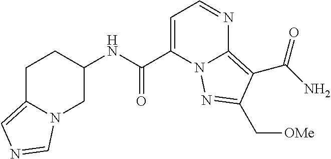 [SMILES: COCc1nn2c(C(=O)NC3CCc4cncn4C3)ccnc2c1C(N)=O]

2-(Methoxymethyl)-N7-(5,6,7,8-tetrahydroimidazo[1,5-a]pyridin-6-yl)pyrazolo[1,5-a]pyrimidine-3,7-dicarboxamide (22 mg) was synthesised following a similar procedure to that described in General Method E.

¹H NMR (400 MHz, MeOD) δ 8.91 (d, J=4.4 Hz, 1H), 7.89 (d, J=4.4 Hz, 1H), 7.57 (d, J=1.1 Hz, 1H), 6.81-6.76 (m, 1H), 4.98 (s, 2H), 4.79-4.72 (m, 1H), 4.40-4.27 (m, 2H), 3.44 (s, 3H), 3.22-2.98 (m, 2H), 2.33-2.16 (m, 2H). Exchangeable protons not observed.

LCMS (Method 7): 1.12 min; 370.2 [M+H]⁺

### Synthesis of Compound 189, N7-(1,1-dioxo-3,4-dihydro-2H-thiochromen-4-yl)-2-(methoxymethyl)pyrazolo[1,5-a]pyrimidine-3,7-dicarboxamide

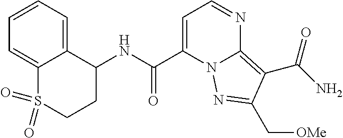 [SMILES: COCc1nn2c(C(=O)NC3CCS(=O)(=O)c4ccccc43)ccnc2c1C(N)=O]

N7-(1,1-dioxo-3,4-dihydro-2H-thiochromen-4-yl)-2-(methoxymethyl)pyrazolo[1,5-a]pyrimidine-3,7-dicarboxamide (34 mg) was synthesised following a similar procedure to that described in General Method E.

¹H NMR (400 MHz, DMSO-d₆) δ 10.28 (d, J=8.2 Hz, 1H), 8.93 (d, J=4.4 Hz, 1H), 7.86 (dd, J=7.8, 1.5 Hz, 1H), 7.80-7.75 (m, 1H), 7.71-7.57 (m, 5H), 5.67-5.55 (m, 1H), 4.95 (s, 2H), 3.90-3.77 (m, 1H), 3.75-3.64 (m, 1H), 3.33 (s, 3H), 2.74-2.63 (m, 1H), 2.62-2.53 (m, 1H)

LCMS (Method 7): 1.88 min, 430.2 [M+H]⁺

### Synthesis of Compound 190, 2-(methoxymethyl)-N7-(4,5,6,7-tetrahydro-1,3-benzothiazol-6-yl)pyrazolo[1,5-a]pyrimidine-3,7-dicarboxamide

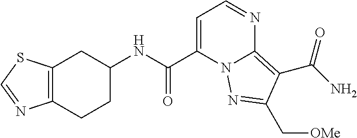 [SMILES: COCc1nn2c(C(=O)NC3CCc4ncsc4C3)ccnc2c1C(N)=O]

2-(Methoxymethyl)-N7-(4,5,6,7-tetrahydro-1,3-benzothiazol-6-yl)pyrazolo[1,5-a]pyrimidine-3,7-dicarboxamide 36 mg) was synthesised following a similar procedure to that described in General Method E.

¹H NMR (400 MHz, CDCl₃) δ 10.81 (d, J=7.4 Hz, 1H), 8.80 (d, J=4.4 Hz, 1H), 8.71 (s, 1H), 8.01 (s, 1H), 7.95 (d, J=4.4 Hz, 1H), 5.59 (s, 1H), 5.06 (s, 2H), 4.79-4.67 (m, 1H), 3.51 (s, 3H), 3.33 (dd, J=16.6, 5.0 Hz, 1H), 3.24-3.01 (m, 3H), 2.30-2.20 (m, 2H)

LCMS (Method 7): 1.80 min, 387.2 [M+H]⁺

### Synthesis of 2-bromo-1-(3,5-difluoro-2-hydroxy-phenyl)ethenone

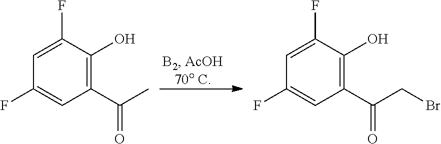

A solution of bromine (279 μL, 5.44 mmol) in AcOH (1.2 mL) was added dropwise to a suspension of 1-(3,5-difluoro-2-hydroxy-phenyl) ethanone (780 mg, 4.53 mmol) in AcOH (5.8 mL), and the reaction was heated at 70° C. in a microwave reactor for 2 h. The reaction mixture was cooled to room temperature and concentrated in vacuo. The crude product was purified by column chromatography (eluting with 0 to 30% EtOAc in heptane) to provide the title compound (1.23 g) as the impure product.

¹H NMR (400 MHz, CDCl₃) δ 11.44 (s, 1H), 7.30-7.24 (m, 1H), 7.20-7.13 (m, 1H), 4.38 (s, 2H)

### Synthesis of 5,7-difluorobenzofuran-3-one

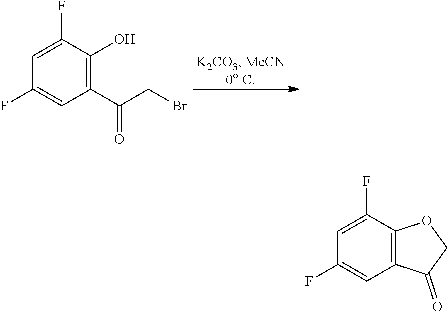 [SMILES: O=C(CBr)c1cc(F)cc(F)c1O ; O=C1COc2c(F)cc(F)cc21]

K₂CO₃ (336 mg, 2.43 mmol) was added to a solution of 2-bromo-1-(3,5-difluoro-2-hydroxy-phenyl) ethanone (407 mg, 1.62 mmol) in MeCN (10.2 mL) at 0° C. and the reaction was stirred at 0° C. for 1 h. The reaction mixture was acidified with 1 M aq. HCl and diluted with EtOAc. The layers were separated, and the aqueous layer was extracted with EtOAc (×2). The combined organic layers were washed with brine, dried through a hydrophobic frit, and concentrated in vacuo to provide the title compound (203 mg). The crude product was taken forward to the next step without further purification.

¹H NMR (400 MHz, CDCl₃) δ 7.23-7.15 (m, 2H), 4.75 (s, 2H)

### Synthesis of 5,7-difluorobenzofuran-3-one oxime

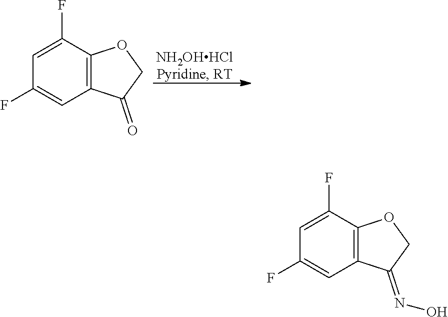 [SMILES: O=C1COc2c(F)cc(F)cc21 ; O/N=C1\COc2c(F)cc(F)cc21]

Hydroxylamine hydrochloride (257 mg, 3.70 mmol) was added to a solution of 5,7-difluorobenzofuran-3-one (315 mg, 1.85 mmol) in pyridine (5.1 mL) and the reaction was stirred at room temperature overnight. The reaction mixture was concentrated in vacuo, and the residue was diluted with EtOAc and water. The layers were separated, and the aqueous layer was extracted with EtOAc (×3). The combined organic layers were dried through a hydrophobic frit and concentrated in vacuo. The crude product was purified by column chromatography (eluting with 0 to 25% EtOAc in heptane) to provide the title compound (135 mg).

¹H NMR (400 MHz, DMSO-d₆) δ 11.71 (s, 1H), 7.41 (ddd, J=11.0, 9.5, 2.5 Hz, 1H), 7.24 (ddd, J=7.5, 2.5, 1.1 Hz, 1H), 5.28 (s, 2H)

### Synthesis of 5,7-difluoro-2,3-dihydrobenzofuran-3-amine

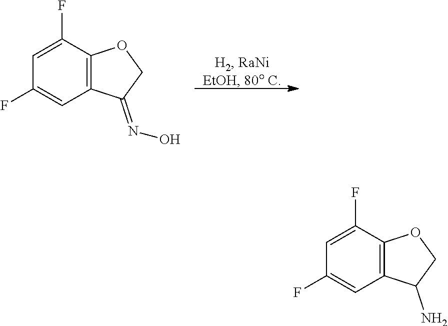 [SMILES: O/N=C1\COc2c(F)cc(F)cc21 ; NC1COc2c(F)cc(F)cc21]

5,7-difluorobenzofuran-3-one oxime (116 mg, 0.63 mmol) was dissolved in EtOH (60 mL) and the solution was hydrogenated over RaNi at 80° C. and 70 bar (H-cube, 1 mL/min). The resultant solution was concentrated in vacuo, and the crude product was purified by SCX chromatography (eluting with 2 M NH₃ in MeOH) to provide the title compound (44 mg).

¹H NMR (400 MHz, CDCl₃) δ 6.87-6.81 (m, 1H), 6.78-6.70 (m, 1H), 4.75 (dd, J=9.4, 8.0 Hz, 1H), 4.66 (dd, J=8.1, 5.0 Hz, 1H), 4.27 (dd, J=9.4, 5.0 Hz, 1H), 1.95 (s, 2H)

### Synthesis of Compound 191, N7-(5,7-difluoro-2,3-dihydrobenzofuran-3-yl)-2-(methoxymethyl)pyrazolo[1,5-a]pyrimidine-3,7-dicarboxamide

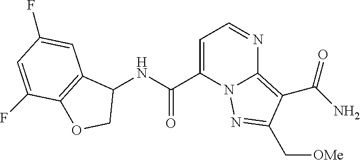 [SMILES: COCc1nn2c(C(=O)NC3COc4c(F)cc(F)cc43)ccnc2c1C(N)=O]

N7-(5,7-difluoro-2,3-dihydrobenzofuran-3-yl)-2-(methoxymethyl)pyrazolo[1,5-a]pyrimidine-3,7-dicarboxamide (44 mg) was synthesised following a similar procedure to that described in General Method E.

¹H NMR (400 MHz, DMSO-d₆) δ 10.42 (d, J=6.9 Hz, 1H), 8.93 (d, J=4.3 Hz, 1H), 7.69 (d, J=4.4 Hz, 1H), 7.65 (s, 1H), 7.60 (s, 1H), 7.38-7.28 (m, 1H), 7.23 (dd, J=7.4, 2.6 Hz, 1H), 5.90-5.82 (m, 1H), 5.03-4.88 (m, 3H), 4.61 (dd, J=9.9, 4.7 Hz, 1H). CH₃ signal obscured by H₂O peak at δ 3.32.

LCMS (Method 7): 2.34 min, 404.2 [M+H]⁺

### Synthesis of 7-fluorobenzofuran-3-one oxime

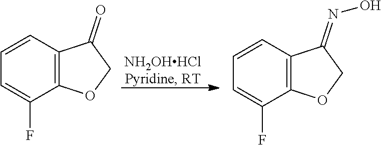 [SMILES: O=C1COc2c(F)cccc21 ; O/N=C1\COc2c(F)cccc21]

Hydroxylamine hydrochloride (91 mg, 1.31 mmol) was added to a solution of 7-fluorobenzofuran-3-one (200 mg, 1.31 mmol) in pyridine (3.6 mL) and the reaction was stirred at room temperature for 19 h. The reaction mixture was concentrated in vacuo, and the residue was diluted with EtOAc and water. The layers were separated, and the aqueous layer was extracted with EtOAc (×2). The combined organic layers were dried through a hydrophobic frit and concentrated in vacuo to provide the title compound (197 mg).

¹H NMR (400 MHz, CDCl₃) δ 7.36 (dd, J=7.7, 1.1 Hz, 1H), 7.13 (ddd, J=10.6, 8.2, 1.1 Hz, 1H), 6.94 (ddd, J=8.2, 7.7, 4.1 Hz, 1H), 5.27 (s, 2H). Exchangeable proton not observed.

LCMS (Method 6): 1.62 min, 168.2 [M+H]⁺

### Synthesis of 7-fluoro-2,3-dihydrobenzofuran-3-amine

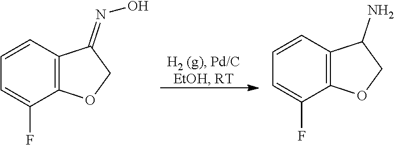 [SMILES: O/N=C1\COc2c(F)cccc21 ; NC1COc2c(F)cccc21]

7-Fluorobenzofuran-3-one oxime (100 mg, 0.60 mmol) was added to a suspension of 10% Pd/C in EtOH (10 mL) and the reaction was stirred at room temperature under a hydrogen atmosphere (balloon) for 18 h. The reaction mixture was filtered through a short pad of DiCalite® and concentrated in vacuo to provide the title compound (78 mg).

¹H NMR (400 MHz, CDCl₃) δ 7.09 (d, J=7.4 Hz, 1H), 6.97 (dd, J=10.6, 8.1 Hz, 1H), 6.88-6.80 (m, 1H), 4.77-4.62 (m, 2H), 4.35-4.23 (m, 1H), 3.02 (s, 2H)

### Synthesis of Compound 192, N7-(7-fluoro-2,3-dihydrobenzofuran-3-yl)-2-(methoxymethyl)pyrazolo[1,5-a]pyrimidine-3,7-dicarboxamide

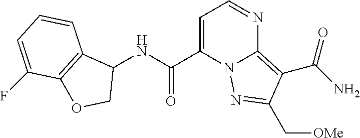 [SMILES: COCc1nn2c(C(=O)NC3COc4c(F)cccc43)ccnc2c1C(N)=O]

N7-(7-fluoro-2,3-dihydrobenzofuran-3-yl)-2-(methoxymethyl)pyrazolo[1,5-a]pyrimidine-3,7-dicarboxamide (19 mg) was synthesised following a similar procedure to that described in General Method E.

¹H NMR (400 MHz, DMSO-d₆) δ 10.41 (s, 1H), 8.93 (d, J=4.4 Hz, 1H), 7.70 (d, J=4.4 Hz, 1H), 7.65 (s, 1H), 7.59 (s, 1H), 7.32 (dt, J=7.5, 1.0 Hz, 1H), 7.27-7.20 (m, 1H), 7.01-6.94 (m, 1H), 5.91-5.81 (m, 1H), 4.98-4.88 (m, 3H), 4.60 (dd, J=9.9, 4.5 Hz, 1H), 3.28 (s, 3H)

LCMS (Method 7): 2.26 min, 386.2 [M+H]⁺

### Synthesis of Compound 193, 2-(methoxymethyl)-N7-[(1S)-tetralin-1-yl]pyrazolo[1,5-a]pyrimidine-3,7-dicarboxamide

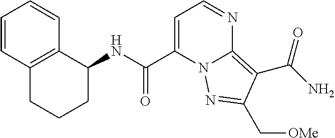 [SMILES: COCc1nn2c(C(=O)N[C@H]3CCCc4ccccc43)ccnc2c1C(N)=O]

2-(Methoxymethyl)-N7-[(1S)-tetralin-1-yl]pyrazolo[1,5-a]pyrimidine-3,7-dicarboxamide (25 mg) was synthesised following a similar procedure to that described in General Method E.

¹H NMR (400 MHz, DMSO-d₆) δ 10.30 (d, J=8.3 Hz, 1H), 8.93 (d, J=4.4 Hz, 1H), 7.73 (d, J=4.4 Hz, 1H), 7.67 (s, 1H), 7.58 (s, 1H), 7.48-7.42 (m, 1H), 7.23-7.13 (m, 3H), 5.33-5.26 (m, 1H), 4.93 (s, 2H), 3.28 (s, 3H), 2.90-2.71 (m, 2H), 2.17-2.08 (m, 1H), 1.99-1.85 (m, 3H)

LCMS (Method 7): 2.54 min, 380.2 [M+H]⁺

### Synthesis of Compound 194, N7-(5-fluoroindan-2-yl)-2-(methoxymethyl)pyrazolo[1,5-a]pyrimidine-3,7-dicarboxamide

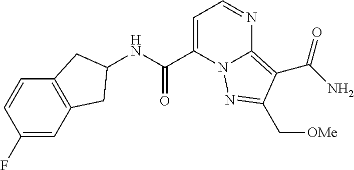 [SMILES: COCc1nn2c(C(=O)NC3Cc4ccc(F)cc4C3)ccnc2c1C(N)=O]

N7-(5-fluoroindan-2-yl)-2-(methoxymethyl)pyrazolo[1,5-a]pyrimidine-3,7-dicarboxamide (39 mg) was synthesised following a similar procedure to that described in General Method E.

¹H NMR (400 MHz, DMSO-d₆) δ 10.21 (d, J=7.1 Hz, 1H), 8.91 (d, J=4.4 Hz, 1H), 7.72-7.63 (m, 2H), 7.58 (s, 1H), 7.34-7.25 (m, 1H), 7.13 (dd, J=9.3, 2.5 Hz, 1H), 7.04-6.95 (m, 1H), 4.92 (s, 2H), 4.87-4.78 (m, 1H), 3.44-3.32 (m, 5H), 3.03-2.88 (m, 2H)

LCMS (Method 7): 2.45 min, 384.2 [M+H]⁺

### Synthesis of Compound 195, N7-(5,6-difluoroindan-2-yl)-2-(methoxymethyl)pyrazolo[1,5-a]pyrimidine-3,7-dicarboxamide

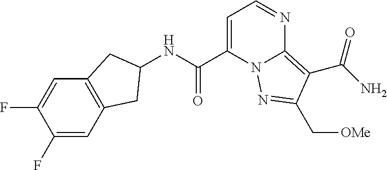 [SMILES: COCc1nn2c(C(=O)NC3Cc4cc(F)c(F)cc4C3)ccnc2c1C(N)=O]

N7-(5,6-difluoroindan-2-yl)-2-(methoxymethyl)pyrazolo[1,5-a]pyrimidine-3,7-dicarboxamide (20 mg) was synthesised following a similar procedure to that described in General Method E.

¹H NMR (400 MHz, DMSO-d₆) δ 10.20 (d, J=7.2 Hz, 1H), 8.91 (d, J=4.4 Hz, 1H), 7.73-7.63 (m, 2H), 7.58 (s, 1H), 7.37 (t, J=9.2 Hz, 2H), 4.92 (s, 2H), 4.87-4.78 (m, 1H), 3.42-3.32 (m, 5H), 2.94 (dd, J=16.4, 4.3 Hz, 2H)

LCMS (Method 7): 2.50 min, 402.2 [M+H]⁺

### Synthesis of t-butyl-6-fluoro-2H-chromene-3-carboxylate

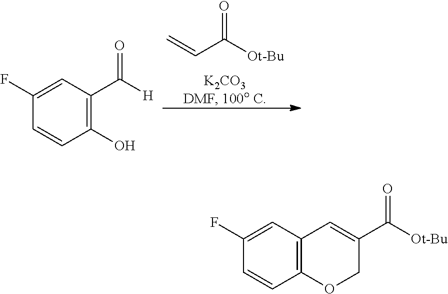

t-Butyl acrylate (13.6 mL, 93.09 mmol) was added to a suspension of 5-fluoro-2-hydroxy-benzaldehyde (5 g, 35.69 mmol) and K₂CO₃ (14.8 g, 107.06 mmol) in DMF (75 mL) and the reaction was heated at 100° C. for 18 h. The reaction mixture was cooled to room temperature and diluted with EtOAc. The organic layer was washed with 5% aq. LiCl (×3) and brine, dried over Na₂SO₄, and concentrated in vacuo. The crude product was purified by column chromatography (eluting with 0 to 100% EtOAc in heptane) to provide the title compound (2.09 g).

¹H NMR (400 MHz, DMSO-d₆) δ 7.39 (s, 1H), 7.26 (dd, J=8.7, 3.1 Hz, 1H), 7.10 (td, J=8.7, 3.1 Hz, 1H), 6.87 (ddd, J=8.9, 4.6, 0.7 Hz, 1H), 4.87 (d, J=1.5 Hz, 2H), 1.49 (s, 9H)

LCMS (Method 7): 3.41 min, 193.2 [M-t-Bu]⁻

### Synthesis of t-butyl 6-fluorochromane-3-carboxylate

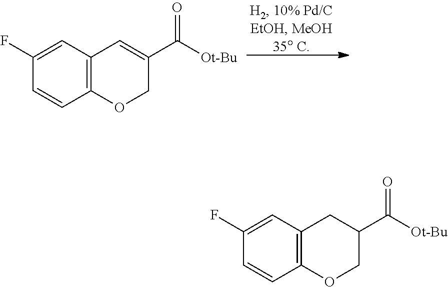 [SMILES: CC(C)(C)OC(=O)C1=Cc2cc(F)ccc2OC1 ; CC(C)(C)OC(=O)C1COc2ccc(F)cc2C1]

t-Butyl 6-fluoro-2H-chromene-3-carboxylate (2.09 g, 8.33 mmol) was dissolved in EtOH (22 mL) and MeOH (8 mL), and the solution was hydrogenated over 10% Pd/C at 35° C. and 10 bar (H-cube, 1 mL/min). The resultant solution was concentrated in vacuo to provide the title compound (838 mg).

¹H NMR (400 MHz, DMSO-d₆) δ 7.02-6.94 (m, 1H), 6.94-6.84 (m, 1H), 6.76 (dd, J=8.9, 5.0 Hz, 1H), 4.30-4.21 (m, 1H), 4.12-4.06 (m, 1H), 2.99-2.89 (m, 3H), 1.39 (s, 9H)

LCMS (Method 7): 3.25 min, 195.2 [M-t-Bu]⁻

### Synthesis of 6-fluorochromane-3-carboxylic Acid

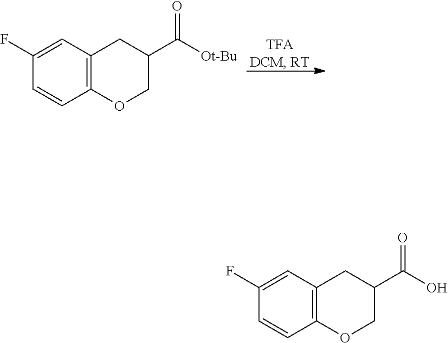 [SMILES: CC(C)(C)OC(=O)C1COc2ccc(F)cc2C1 ; O=C(O)C1COc2ccc(F)cc2C1]

TFA (1.18 mL, 15.47 mmol) was added to a solution of t-butyl 6-fluorochromane-3-carboxylate (838 mg, 3.16 mmol) in DCM (5 mL), and the reaction was stirred at room temperature for 2 h. The reaction mixture was concentrated in vacuo, and the residue was dissolved in DCM and filtered. The filtrate was left to stand overnight, and the resulting precipitate was collected by filtration, washed with heptane, and dried to provide the title compound (390 mg).

¹H NMR (400 MHz, DMSO-d₆) δ 12.67 (s, 1H), 6.98 (dd, J=9.2, 3.1 Hz, 1H), 6.93-6.86 (m, 1H), 6.76 (dd, J=8.9, 5.0 Hz, 1H), 4.32-4.23 (m, 1H), 4.15-4.09 (m, 1H), 3.01-2.92 (m, 3H)

LCMS (Method 7): 2.15 min, 195.2 [M−H]⁻

### Synthesis of 6-fluorochroman-3-amine

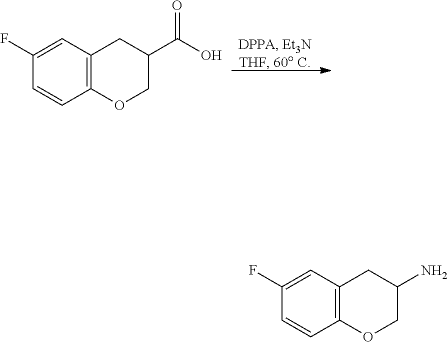 [SMILES: O=C(O)C1COc2ccc(F)cc2C1 ; NC1COc2ccc(F)cc2C1]

Diphenylphosphoryl azide (513 μL, 2.39 mmol) and Et₃N (690 μL, 4.98 mmol) were added to a solution of 6-fluorochromane-3-carboxylic acid (390 mg, 1.99 mmol) in THF (2.5 mL), and the reaction was heated at 60° C. for 2 h. The reaction mixture was cooled to 0° C. and acidified with 5 M aq. HCl. The mixture was warmed to room temperature, stirred for 16 h, then diluted with DCM. The layers were separated, and the aqueous layer was extracted with DCM (×2). The aqueous layer was basified with 2 M aq. NaOH and extracted with DCM (×3). The combined organic layers were dried through a hydrophobic frit and concentrated in vacuo to provide the title compound (49 mg).

¹H NMR (400 MHz, DMSO-d₆) δ 6.93-6.84 (m, 2H), 6.73 (dd, J=8.7, 5.0 Hz, 1H), 4.05 (ddd, J=10.4, 3.5, 1.9 Hz, 1H), 3.57 (dd, J=10.3, 8.5 Hz, 1H), 3.14-3.06 (m, 1H), 2.92-2.85 (m, 1H), 2.48-2.39 (m, 1H), 1.65 (s, 2H)

LCMS (Method 7): 0.14 min, 168.2 [M+H]⁺

### Synthesis of Compound 196, N7-(6-fluorochroman-3-yl)-2-(methoxymethyl)pyrazolo[1,5-a]pyrimidine-3,7-dicarboxamide

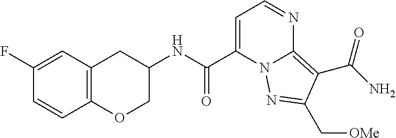 [SMILES: COCc1nn2c(C(=O)NC3COc4ccc(F)cc4C3)ccnc2c1C(N)=O]

N7-(6-fluorochroman-3-yl)-2-(methoxymethyl)pyrazolo[1,5-a]pyrimidine-3,7-dicarboxamide (15 mg) was synthesised following a similar procedure to that described in General Method E.

¹H NMR (400 MHz, DMSO-d₆) δ 10.32 (d, J=7.4 Hz, 1H), 8.92 (d, J=4.4 Hz, 1H), 7.77 (d, J=4.4 Hz, 1H), 7.67 (s, 1H), 7.58 (s, 1H), 7.05-6.94 (m, 2H), 6.87 (dd, J=8.9, 4.9 Hz, 1H), 4.87-4.74 (m, 2H), 4.64-4.56 (m, 1H), 4.27-4.19 (m, 2H), 3.26 (s, 4H), 2.88 (dd, J=17.1, 3.9 Hz, 1H)

LCMS (Method 7): 2.30 min, 400.2 [M+H]⁺

### Synthesis of Compound 197, N7-[(4-hydroxyphenyl)methyl]-2-(methoxymethyl)pyrazolo[1,5-a]pyrimidine-3,7-dicarboxamide

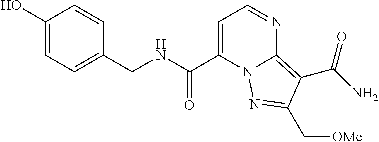 [SMILES: COCc1nn2c(C(=O)NCc3ccc(O)cc3)ccnc2c1C(N)=O]

N7-[(4-hydroxyphenyl)methyl]-2-(methoxymethyl)pyrazolo[1,5-a]pyrimidine-3,7-dicarboxamide (20 mg) was synthesised following a similar procedure to that described in General Method E.

¹H NMR (400 MHz, DMSO-d₆) δ 10.18 (t, J=5.6 Hz, 1H), 9.37 (s, 1H), 8.92 (d, J=4.4 Hz, 1H), 7.73-7.67 (m, 2H), 7.59 (s, 1H), 7.29-7.21 (m, 2H), 6.79-6.71 (m, 2H), 4.97 (s, 2H), 4.55 (d, J=5.6 Hz, 2H), 3.35 (s, 3H)

LCMS (Method 7): 1.87 min, 356.2 [M+H]⁺

### Synthesis of Compound 198, 2-(methoxymethyl)-N7-[(1S)-3,3-dimethylindan-1-yl]pyrazolo[1,5-a]pyrimidine-3,7-dicarboxamide

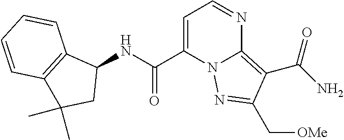 [SMILES: COCc1nn2c(C(=O)N[C@H]3CC(C)(C)c4ccccc43)ccnc2c1C(N)=O]

2-(Methoxymethyl)-N7-[(1S)-3,3-dimethylindan-1-yl]pyrazolo[1,5-a]pyrimidine-3,7-dicarboxamide (61 mg) was synthesised following a similar procedure to that described in General Method E.

¹H NMR (400 MHz, DMSO-d₆) δ 10.26 (d, J=7.7 Hz, 1H), 8.94 (d, J=4.4 Hz, 1H), 7.74 (d, J=4.4 Hz, 1H), 7.71-7.66 (m, 1H), 7.59 (s, 1H), 7.45 (d, J=7.5, 1.1 Hz, 1H), 7.36-7.28 (m, 2H), 7.27-7.20 (m, 1H), 5.67-5.56 (m, 1H), 4.94 (s, 2H), 3.30 (s, 3H), 2.49-2.47 (m, 1H), 1.93-1.83 (m, 1H), 1.40 (s, 3H), 1.26 (s, 3H)

LCMS (Method 7): 2.72 min, 394.2 [M+H]⁺

### Synthesis of Compound 199, 2-(methoxymethyl)-N7-[(2-methoxyphenyl)methyl]pyrazolo[1,5-a]pyrimidine-3,7-dicarboxamide

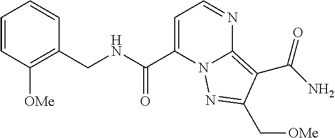 [SMILES: COCc1nn2c(C(=O)NCc3ccccc3OC)ccnc2c1C(N)=O]

2-(methoxymethyl)-N7-[(2-methoxyphenyl)methyl]pyrazolo[1,5-a]pyrimidine-3,7-dicarboxamide (25 mg) was synthesised following a similar procedure to that described in General Method E.

¹H NMR (400 MHz, DMSO-d₆) δ 10.35 (t, J=5.8 Hz, 1H), 8.93 (d, J=4.4 Hz, 1H), 7.75 (d, J=4.4 Hz, 1H), 7.72 (s, 1H), 7.60 (s, 1H), 7.43 (dd, J=7.4, 1.7 Hz, 1H), 7.31 (ddd, J=8.2, 7.4, 1.7 Hz, 1H), 7.05 (dd, J=8.3, 1.1 Hz, 1H), 6.94 (td, J=7.4, 1.1 Hz, 1H), 5.00 (s, 2H), 4.62 (d, J=5.7 Hz, 2H), 3.88 (s, 3H), 3.37 (s, 3H)

LCMS (Method 7): 2.33 min, 370.2 [M+H]⁺

### Synthesis of Compound 200, N7-(cis-1-hydroxyindan-2-yl)-2-(methoxymethyl)pyrazolo[1,5-a]pyrimidine-3,7-dicarboxamide

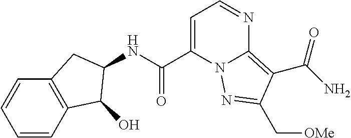 [SMILES: COCc1nn2c(C(=O)N[C@@H]3Cc4ccccc4[C@@H]3O)ccnc2c1C(N)=O]

N7-(cis-1-hydroxyindan-2-yl)-2-(methoxymethyl)pyrazolo[1,5-a]pyrimidine-3,7-dicarboxamide (20 mg) was synthesised following a similar procedure to that described in General Method E.

¹H NMR (400 MHz, DMSO-d₆) δ 10.54 (d, J=7.2 Hz, 1H), 8.95 (d, J=4.4 Hz, 1H), 7.84 (d, J=4.4 Hz, 1H), 7.72 (s, 1H), 7.59 (s, 1H), 7.45-7.38 (m, 1H), 7.31-7.21 (m, 3H), 5.73 (d, J=5.6 Hz, 1H), 5.10 (t, J=5.6 Hz, 1H), 4.91 (d, J=0.6 Hz, 2H), 4.75-4.64 (m, 1H), 3.33 (s, 3H), 3.28 (d, J=7.3 Hz, 1H), 3.04-2.94 (m, 1H)

LCMS (Method 7): 2.06 min, 382.2 [M+H]⁺

### Synthesis of Compound 201, 2-(methoxymethyl)-N7-cis-1-methylindan-2-yl]pyrazolo[1,5-a]pyrimidine-3,7-dicarboxamide

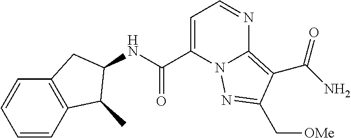 [SMILES: COCc1nn2c(C(=O)N[C@@H]3Cc4ccccc4[C@@H]3C)ccnc2c1C(N)=O]

2-(Methoxymethyl)-N7-cis-1-methylindan-2-yl]pyrazolo[1,5-a]pyrimidine-3,7-dicarboxamide (41 mg) was synthesised following a similar procedure to that described in General Method E.

¹H NMR (400 MHz, DMSO-d₆) δ 10.15 (d, J=8.0 Hz, 1H), 8.92 (d, J=4.4 Hz, 1H), 7.73 (d, J=4.4 Hz, 1H), 7.67 (s, 1H), 7.58 (s, 1H), 7.32-7.25 (m, 2H), 7.24-7.14 (m, 2H), 4.93-4.81 (m, 3H), 3.55-3.43 (m, 1H), 3.28 (d, J=6.9 Hz, 1H), 3.23 (s, 3H), 2.94 (dd, J=16.1, 4.6 Hz, 1H), 1.31 (d, J=7.3 Hz, 3H)

LCMS (Method 7): 2.53 min, 380.1 [M+H]⁺

### Synthesis of tert-butyl N-[trans-3-(trifluoromethoxy)cyclobutyl]carbamate

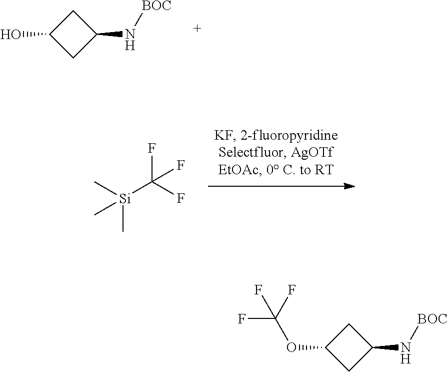

Selectfluor™ (1-(Chloromethyl)-4-fluoro-1,4-diazabicyclo[2.2.2]octane-1,4-diium ditetrafluoroborate; 6.75 g, 18.94 mmol), AgOTf (14.11 g, 54.90 mmol), and KF (3.19 g, 54.90 mmol) were added to a solution of tert-butyl N-(trans-3-hydroxycyclobutyl)carbamate (2.57 g, 13.73 mmol) in EtOAc (51 mL), followed by the dropwise addition of 2-fluoropyridine (3.6 mL, 41.80 mmol) and trimethyl(trifluoromethyl) silane (5.1 mL, 34.31 mmol). The reaction was stirred at room temperature for 72 h, then the mixture was filtered through a short pad of Dicalite® and concentrated in vacuo. The residue was dissolved in EtOAc and the solution was washed with 10% aq. CuSO₄, dried through a hydrophobic frit, and concentrated in vacuo. The crude product was purified by column chromatography (eluting with 0 to 100% EtOAc in heptane) to provide the title compound (2.57 g).

¹H NMR (400 MHz, CDCl₃) δ 4.88-4.79 (m, 1H), 4.70 (br s, 1H), 4.24 (br s, 1H), 2.68-2.57 (m, 2H), 2.43-2.32 (m, 2H), 1.46 (s, 9H)

### Synthesis of trans-3-(trifluoromethoxy)cyclobutanamine;hydrochloride

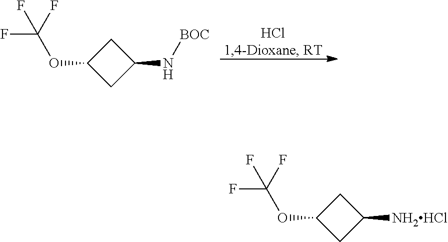

HCl (4 M in 1,4-dioxane) (6.4 mL, 25.47 mmol) was added to a solution of tert-butyl N-[trans-3-(trifluoromethoxy)cyclobutyl]carbamate (1.30 g, 5.09 mmol) in DCM (39 mL) and the reaction was stirred at room temperature for 18 h then concentrated in vacuo. The residue was redissolved in DCM and concentrated in vacuo. The process was repeated three times to provide the title compound (952 mg).

¹H NMR (400 MHz, DMSO-d₆) δ 8.49 (s, 3H), 5.27-5.15 (m, 1H), 3.86-3.72 (m, 1H), 2.64-2.53 (m, 4H)

### Synthesis of Compound 224, 2-(methoxymethyl)-N7-[trans-3-(trifluoromethoxy)cyclobutyl]pyrazolo[1,5-a]pyrimidine-3,7-dicarboxamide

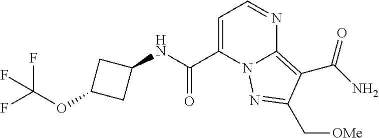 [SMILES: COCc1nn2c(C(=O)N[C@H]3C[C@H](OC(F)(F)F)C3)ccnc2c1C(N)=O]

2-(Methoxymethyl)-N7-[trans-3-(trifluoromethoxy)cyclobutyl]pyrazolo[1,5-a]pyrimidine-3,7-dicarboxamide (63 mg) was synthesised following a similar procedure to that described in General Method E.

¹H NMR (400 MHz, DMSO-d₆) δ 10.12 (d, J=6.5 Hz, 1H), 8.92 (d, J=4.3 Hz, 1H), 7.72-7.56 (m, 3H), 5.11-5.03 (m, 1H), 5.00 (s, 2H), 4.60-4.50 (m, 1H), 3.43 (s, 3H), 2.76-2.65 (m, 2H), 2.61-2.47 (m, 2H)

LCMS (Method 7): 2.36 min, 388.2 [M+H]⁺

### Synthesis of Compound 225, N7-(1,1-dioxothietan-3-yl)-2-(methoxymethyl) pyrazolo[1,5-a]pyrimidine-3,7-dicarboxamide

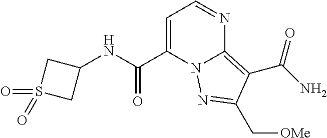 [SMILES: COCc1nn2c(C(=O)NC3CS(=O)(=O)C3)ccnc2c1C(N)=O]

N7-(1,1-dioxothietan-3-yl)-2-(methoxymethyl)pyrazolo[1,5-a]pyrimidine-3,7-dicarboxamide (41 mg) was synthesised following a similar procedure to that described in General Method E.

¹H NMR (400 MHz, DMSO-d₆) δ 10.48 (d, J=4.7 Hz, 1H), 8.95 (d, J=4.3 Hz, 1H), 7.72-7.65 (m, 2H), 7.61 (s, 1H), 4.98 (s, 2H), 4.80-4.69 (m, 3H), 4.30-4.21 (m, 2H), 3.42 (s, 3H)

LCMS (Method 7): 1.43 min, 354.14 [M+H]⁺

### Synthesis of Compound 226, N7-(4,4-difluorotetralin-1-yl)-2-(methoxymethyl)-5-methyl-pyrazolo[1,5-a]pyrimidine-3,7-dicarboxamide

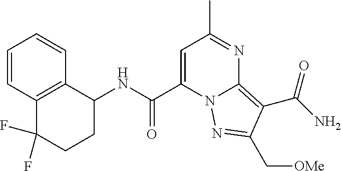 [SMILES: COCc1nn2c(C(=O)NC3CCC(F)(F)c4ccccc43)cc(C)nc2c1C(N)=O]

N7-(4,4-difluorotetralin-1-yl)-2-(methoxymethyl)-5-methyl-pyrazolo[1,5-a]pyrimidine-3,7-dicarboxamide (32 mg) was synthesised following a similar procedure to that described in General Method E.

¹H NMR (400 MHz, DMSO-d₆) δ 10.26 (d, J=8.3 Hz, 1H), 7.76-7.70 (m, 1H), 7.69-7.64 (m, 2H), 7.61 (s, 1H), 7.58-7.47 (m, 3H), 5.47-5.38 (m, 1H), 4.91 (s, 2H), 3.30 (s, 3H), 2.72 (s, 3H), 2.59-2.51 (m, 2H), 2.37-2.25 (m, 1H), 2.13-2.00 (m, 1H)

LCMS (Method 7): 2.61 min, 430.2 [M+H]⁺

### Synthesis of Compound 227, N7-indan-2-yl-2-(methoxymethyl)-5-methyl-pyrazolo[1,5-a]pyrimidine-3,7-dicarboxamide

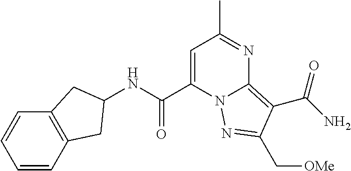 [SMILES: COCc1nn2c(C(=O)NC3Cc4ccccc4C3)cc(C)nc2c1C(N)=O]

N7-indan-2-yl-2-(methoxymethyl)-5-methyl-pyrazolo[1,5-a]pyrimidine-3,7-dicarboxamide (90 mg) was synthesised following a similar procedure to that described in General Method E.

¹H NMR (400 MHz, DMSO-d₆) δ 10.21 (d, J=7.0 Hz, 1H), 7.75-7.71 (m, 1H), 7.61 (s, 1H), 7.55-7.48 (m, 1H), 7.32-7.24 (m, 2H), 7.22-7.14 (m, 2H), 4.88 (s, 2H), 4.84-4.74 (m, 1H), 3.38 (dd, J=16.2, 7.2 Hz, 2H), 3.29 (s, 3H), 2.96 (dd, J=16.2, 4.6 Hz, 2H), 2.70 (s, 3H)

LCMS (Method 7): 2.48 min, 380.2 [M+H]⁺

### Synthesis of Compound 228, N7-(1,1-dioxo-2,3-dihydrobenzothiophen-3-yl)-2-isopropyl-pyrazolo[1,5-a]pyrimidine-3,7-dicarboxamide

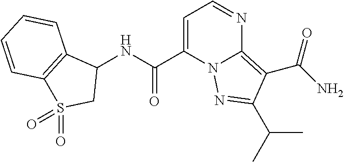 [SMILES: CC(C)c1nn2c(C(=O)NC3CS(=O)(=O)c4ccccc43)ccnc2c1C(N)=O]

N7-(1,1-dioxo-2,3-dihydrobenzothiophen-3-yl)-2-isopropyl-pyrazolo[1,5-a]pyrimidine-3,7-dicarboxamide (143 mg) was synthesised following a similar procedure to that described in General Method E.

¹H NMR (400 MHz, DMSO-d₆) δ 10.73 (d, J=7.7 Hz, 1H), 8.91 (d, J=4.4 Hz, 1H), 7.92-7.88 (m, 1H), 7.86-7.69 (m, 5H), 7.56-7.45 (m, 1H), 6.03-5.95 (m, 1H), 4.18 (dd, J=13.7, 7.4 Hz, 1H), 3.99-3.88 (m, 1H), 3.74 (dd, J=13.7, 4.4 Hz, 1H), 1.19 (d, J=6.9 Hz, 3H), 1.17 (d, J=6.9 Hz, 3H)

LCMS (Method 7): 2.23 min, 414.2 [M+H]⁺

### Synthesis of Compound 229, 2-cyclopropyl-N7-(1,1-dioxo-3,4-dihydro-2H-thiochromen-4-yl)pyrazolo[1,5-a]pyrimidine-3,7-dicarboxamide

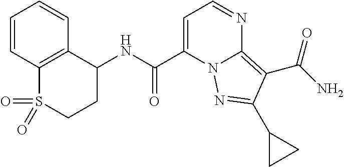 [SMILES: NC(=O)c1c(C2CC2)nn2c(C(=O)NC3CCS(=O)(=O)c4ccccc43)ccnc12]

2-Cyclopropyl-N7-(1,1-dioxo-3,4-dihydro-2H-thiochromen-4-yl)pyrazolo[1,5-a]pyrimidine-3,7-dicarboxamide (210 mg) was synthesised following a similar procedure to that described in General Method E.

¹H NMR (400 MHz, DMSO-d₆) δ 10.08 (d, J=8.1 Hz, 1H), 8.85 (d, J=4.4 Hz, 1H), 7.90-7.86 (m, 1H), 7.74 (s, 1H), 7.72-7.62 (m, 3H), 7.57 (d, J=4.4 Hz, 1H), 7.53 (s, 1H), 5.59-5.52 (m, 1H), 3.85-3.75 (m, 1H), 3.72-3.62 (m, 1H), 3.20-3.12 (m, 1H), 2.71-2.54 (m, 2H), 1.10-1.00 (m, 2H), 0.90-0.79 (m, 2H)

LCMS (Method 7): 2.10 min, 426.0 [M+H]⁺

### Synthesis of Compound 230, N7-(1,1-dioxo-3,4-dihydro-2H-thiochromen-4-yl)-2-isopropyl-pyrazolo[1,5-a]pyrimidine-3,7-dicarboxamide

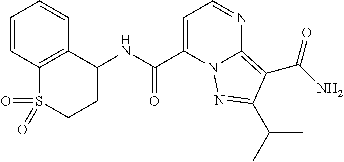 [SMILES: CC(C)c1nn2c(C(=O)NC3CCS(=O)(=O)c4ccccc43)ccnc2c1C(N)=O]

N7-(1,1-dioxo-3,4-dihydro-2H-thiochromen-4-yl)-2-isopropyl-pyrazolo[1,5-a]pyrimidine-3,7-dicarboxamide (130 mg) was synthesised following a similar procedure to that described in General Method E.

¹H NMR (400 MHz, DMSO-d₆) δ 10.33 (d, J=8.0 Hz, 1H), 8.89 (d, J=4.4 Hz, 1H), 7.89-7.85 (m, 1H), 7.81-7.73 (m, 2H), 7.70-7.60 (m, 3H), 7.53-7.43 (m, 1H), 5.63-5.54 (m, 1H), 4.00-3.89 (m, 1H), 3.87-3.76 (m, 1H), 3.73-3.62 (m, 1H), 2.75-2.52 (m, 2H), 1.21 (dd, J=6.9, 1.4 Hz, 6H)

LCMS (Method 7): 2.22 min, 428.2 [M+H]⁺

### Synthesis of Compound 231, 2-cyclopropyl-N7-(1,1-dioxo-2,3-dihydrobenzothiophen-3-yl)pyrazolo[1,5-a]pyrimidine-3,7-dicarboxamide

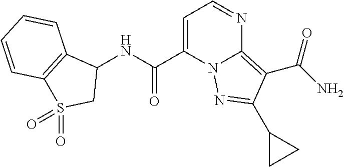 [SMILES: NC(=O)c1c(C2CC2)nn2c(C(=O)NC3CS(=O)(=O)c4ccccc43)ccnc12]

2-Cyclopropyl-N7-(1,1-dioxo-2,3-dihydrobenzothiophen-3-yl)pyrazolo[1,5-a]pyrimidine-3,7-dicarboxamide (185 mg) was synthesised following a similar procedure to that described in General Method E.

¹H NMR (400 MHz, DMSO-d₆) δ 10.47 (d, J=7.8 Hz, 1H), 8.89 (d, J=4.4 Hz, 1H), 7.91 (d, J=7.7 Hz, 1H), 7.87-7.67 (m, 5H), 7.56 (s, 1H), 6.02-5.92 (m, 1H), 4.16 (dd, J=13.7, 7.4 Hz, 1H), 3.73 (dd, J=13.7, 4.5 Hz, 1H), 3.20-3.11 (m, 1H), 1.07-0.97 (m, 2H), 0.96-0.87 (m, 1H), 0.88-0.80 (m, 1H)

LCMS (Method 7): 2.12 min, 412.1 [M+H]⁺

### Synthesis of Compound 232, N7-(3,3-difluoroindan-1-yl)-2-(methoxymethyl)-5-methyl-pyrazolo[1,5-a]pyrimidine-3,7-dicarboxamide

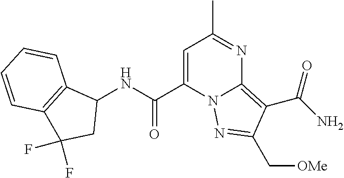 [SMILES: COCc1nn2c(C(=O)NC3CC(F)(F)c4ccccc43)cc(C)nc2c1C(N)=O]

N7-(3,3-difluoroindan-1-yl)-2-(methoxymethyl)-5-methyl-pyrazolo[1,5-a]pyrimidine-3,7-dicarboxamide (45 mg) was synthesised following a similar procedure to that described in General Method E.

¹H NMR (400 MHz, DMSO-d₆) δ 10.35 (d, J=7.6 Hz, 1H), 7.75-7.71 (m, 1H), 7.70-7.64 (m, 3H), 7.63 (s, 1H), 7.60-7.52 (m, 2H), 5.76-5.66 (m, 1H), 4.91 (d, J=2.8 Hz, 2H), 3.30 (s, 3H), 3.28-3.19 (m, 1H), 2.72 (s, 3H), 2.69-2.56 (m, 1H)

LCMS (Method 7): 2.54 min, 416.2 [M+H]⁺

### Synthesis of Compounds 233 and 234, N7-[1,1-dioxo-3,4-dihydro-2H-thiochromen-4-yl]-2-(methoxymethyl)pyrazolo[1,5-a]pyrimidine-3,7-dicarboxamide

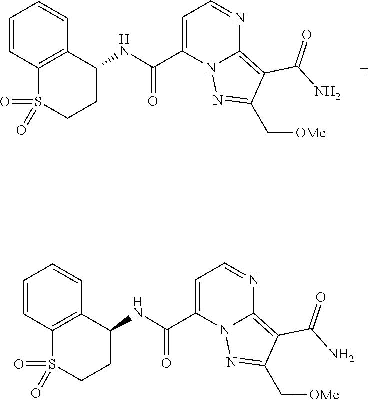 [SMILES: COCc1nn2c(C(=O)N[C@@H]3CCS(=O)(=O)c4ccccc43)ccnc2c1C(N)=O ; COCc1nn2c(C(=O)N[C@H]3CCS(=O)(=O)c4ccccc43)ccnc2c1C(N)=O]

Racemic N7-[1,1-dioxo-3,4-dihydro-2H-thiochromen-4-yl]-2-(methoxymethyl)pyrazolo[1,5-a]pyrimidine-3,7-dicarboxamide (50 mg) was separated by preparative SFC (eluting with 35% MeOH in CO₂) to provide N7-[(4R)-1,1-dioxo-3,4-dihydro-2H-thiochromen-4-yl]-2-(methoxymethyl)pyrazolo[1,5-a]pyrimidine-3,7-dicarboxamidfe (13 mg, early eluting enantiomer, 3.51 min) and N7-[(4S)-1,1-dioxo-3,4-dihydro-2H-thiochromen-4-yl]-2-(methoxymethyl)pyrazolo[1,5-a]pyrimidine-3,7-dicarboxamide (14 mg, late eluting enantiomer, 5.16 min).

¹H NMR (400 MHz, MeOD) δ 8.93 (d, J=4.4 Hz, 1H), 7.93 (dd, J=7.7, 1.6 Hz, 1H), 7.88 (d, J=4.4 Hz, 1H), 7.75-7.69 (m, 1H), 7.69-7.56 (m, 2H), 5.68 (dd, J=7.3, 4.8 Hz, 1H), 5.03 (d, J=0.7 Hz, 2H), 3.74 (ddd, J=14.3, 9.0, 3.5 Hz, 1H), 3.64 (ddd, J=14.3, 8.8, 3.4 Hz, 1H), 3.40 (s, 3H), 2.94-2.73 (m, 2H). Exchangeable protons not observed.

LCMS (Method 7) 1.60 min, 430.2 [M+H]⁺

### Synthesis of Compound 235, 2-cyclopropyl-N7-[(4R)-1,1-dioxo-3,4-dihydro-2H-thiochromen-4-yl]pyrazolo[1,5-a]pyrimidine-3,7-dicarboxamide

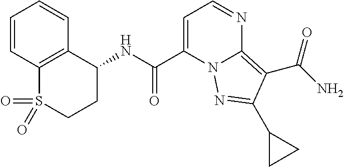 [SMILES: NC(=O)c1c(C2CC2)nn2c(C(=O)N[C@@H]3CCS(=O)(=O)c4ccccc43)ccnc12]

2-Cyclopropyl-N7-[(4R)-1,1-dioxo-3,4-dihydro-2H-thiochromen-4-yl]pyrazolo[1,5-a]pyrimidine-3,7-dicarboxamide (21 mg) was synthesised following a similar procedure to that described in General Method E.

¹H NMR (400 MHz, DMSO-d₆) δ 10.08 (d, J=8.1 Hz, 1H), 8.85 (d, J=4.4 Hz, 1H), 7.88 (d, J=7.8 Hz, 1H), 7.82-7.45 (m, 6H), 5.72-5.38 (m, 1H), 3.85-3.75 (m, 1H), 3.71-3.61 (m, 1H), 3.22-3.11 (m, 1H), 2.77-2.57 (m, 2H), 1.15-1.00 (m, 2H), 0.94-0.76 (m, 2H)

LCMS (Method 7): 2.11 min; 426.2 [M+H]⁺

### Synthesis of Compound 236, 2-cyclopropyl-N7-[(4S)-1,1-dioxo-3,4-dihydro-2H-thiochromen-4-yl]pyrazolo[1,5-a]pyrimidine-3,7-dicarboxamide

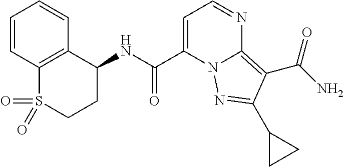 [SMILES: NC(=O)c1c(C2CC2)nn2c(C(=O)N[C@H]3CCS(=O)(=O)c4ccccc43)ccnc12]

2-Cyclopropyl-N7-[(4S)-1,1-dioxo-3,4-dihydro-2H-thiochromen-4-yl]pyrazolo[1,5-a]pyrimidine-3,7-dicarboxamide (14 mg) was synthesised following a similar procedure to that described in General Method E.

¹H NMR (400 MHz, DMSO-d₆) δ 10.08 (d, J=8.0 Hz, 1H), 8.85 (d, J=4.5 Hz, 1H), 7.88 (d, J=7.7 Hz, 1H), 7.82-7.47 (m, 6H), 5.67-5.45 (m, 1H), 3.87-3.74 (m, 1H), 3.73-3.61 (m, 1H), 3.21-3.10 (m, 1H), 2.69-2.55 (m, 2H), 1.12-0.99 (m, 2H), 0.93-0.77 (m, 2H)

LCMS (Method 7): 2.11 min, 426.1 [M+H]⁺

### Synthesis of 6-fluoroindan-1-one oxime

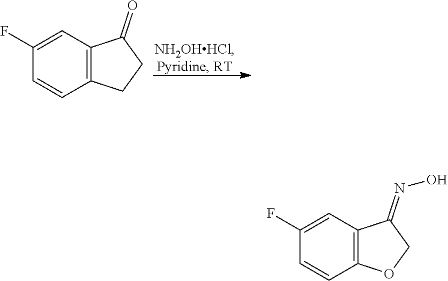 [SMILES: O=C1CCc2ccc(F)cc21 ; O/N=C1\COc2ccc(F)cc21]

Hydroxylamine hydrochloride (1.02 g, 14.65 mmol) was added to a solution of 6-fluoroindan-1-one (2 g, 13.32 mmol) in pyridine (15 mL) and the reaction was stirred at room temperature for 3 h. The reaction mixture was diluted with EtOAc and acidified to pH 2 with 5 M aq. HCl. The layers were separated, and the organic layer was washed successively with 1 M aq. HCl, 10% aq. CuSO₄, and brine, dried through a hydrophobic frit, and concentrated in vacuo. The crude product was purified by column chromatography (eluting with 0 to 100% EtOAc in heptane) to provide the title compound (1.94 g).

¹H NMR (400 MHz, MeOD) δ 7.36-7.25 (m, 2H), 7.07 (ddd, J=9.1, 8.4, 2.6 Hz, 1H), 3.04-2.98 (m, 2H), 2.96-2.91 (m, 2H). Exchangeable proton not observed.

LCMS (Method 6): 1.64 mins, 166.3 [M+H]⁺

### Synthesis of 6-fluoroindan-1-amine

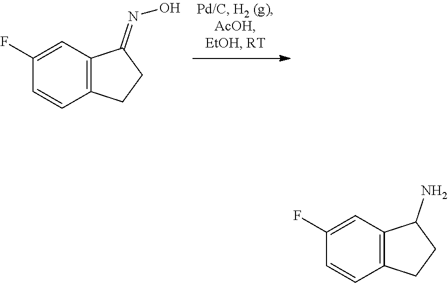 [SMILES: O/N=C1\CCc2ccc(F)cc21 ; NC1CCc2ccc(F)cc21]

10% Pd/C (166 mg, 0.16 mmol) was added to a solution of 6-fluoroindan-1-one oxime (1.66 g, 10.05 mmol) in EtOH (100 mL) under an atmosphere of argon. A balloon of hydrogen was attached to the flask and the reaction was stirred at room temperature for 20 h. An additional portion of 10% Pd/C (166 mg, 0.16 mmol) was added and the flask was recharged with hydrogen. The reaction was stirred at room temperature for a further 3 d. The reaction mixture was filtered through a short pad of Dicalite® and concentrated in vacuo. The residue was dissolved in EtOH (100 mL) and AcOH (10 mL) and 10% Pd/C (166 mg, 0.16 mmol) was added to the solution under an atmosphere of argon. A balloon of hydrogen was attached to the flask and the reaction was stirred at room temperature for 20 h. The reaction mixture was filtered through a short pad of Dicalite® and concentrated to approximately 30 mL in vacuo. The solution was basified to pH 8 with sat. aq. NaHCO₃ and diluted with EtOAc. The layers were separated, and the aqueous layer was extracted with EtOAc (×2) and a 1:9 mixture of MeOH:EtOAc. The combined organic layers were dried through a hydrophobic frit and concentrated in vacuo to provide the title compound (1.47 g). The crude product was taken forward to the next step without purification.

¹H NMR (400 MHz, CDCl₃) δ 7.18-7.11 (m, 1H), 7.07-6.96 (m, 1H), 6.93-6.83 (m, 1H), 4.41-4.26 (m, 1H), 2.98-2.69 (m, 2H), 2.60-2.46 (m, 1H), 1.79-1.66 (m, 1H).

Exchangeable protons not observed.

### Synthesis of 2,2,2-trifluoro-N-(6-fluoroindan-1-yl) acetamide

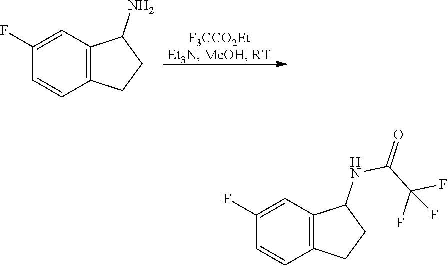 [SMILES: NC1CCc2ccc(F)cc21 ; O=C(NC1CCc2ccc(F)cc21)C(F)(F)F]

Ethyl trifluoroacetate (1.38 mL, 11.63 mmol) was added to a solution of 6-fluoroindan-1-amine (1.47 g, 9.69 mmol) and Et₃N (2.01 mL, 14.54 mmol) in MeOH (20 mL) at 0° C. The reaction was stirred at room temperature for 18 h then concentrated in vacuo. The residue was dissolved in EtOAc and washed with water. The organic layer was dried through a hydrophobic frit and concentrated in vacuo. The crude product was purified by column chromatography (eluting with 0 to 50% EtOAc in heptane) to provide the title compound (1.63 g).

¹H NMR (400 MHz, CDCl₃) δ 7.25-7.19 (m, 1H), 7.02-6.95 (m, 2H), 6.41 (s, 1H), 5.49 (q, J=7.7 Hz, 1H), 3.08-2.96 (m, 1H), 2.95-2.83 (m, 1H), 2.76-2.63 (m, 1H), 2.03-1.89 (m, 1H)

LCMS (Method 6): 1.97 min, 246.0 [M−H]⁻

### Synthesis of 2,2,2-trifluoro-N-(6-fluoro-3-oxo-indan-1-yl) acetamide

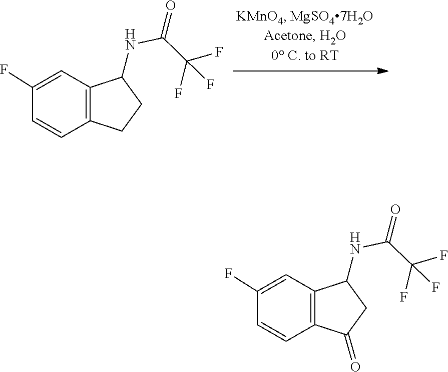 [SMILES: O=C(NC1CCc2ccc(F)cc21)C(F)(F)F ; O=C1CC(NC(=O)C(F)(F)F)c2cc(F)ccc21]

A solution of MgSO₄·7H₂O (3.24 g, 13.15 mmol) in water (10 mL) was added to a solution of 2,2,2-trifluoro-N-(6-fluoroindan-1-yl) acetamide (1.63 g, 6.57 mmol) in acetone (20 mL) and the mixture was cooled to 0° C. KMnO₄ (3.12 g, 19.72 mmol) was added, and the reaction was warmed to room temperature and stirred for 23 h. An additional portion of KMnO₄ (1.04 g, 6.57 mmol) was added, and the reaction was stirred at room temperature for a further 6 h. A solution of Na₂S₂O₃·5H₂O (3.26 g, 13.15 mmol) in water (10 mL) was added, and the mixture was stirred for 10 min. The reaction mixture was filtered through a short pad of Dicalite® and washed with EtOAc and water. The layers were separated, and the aqueous layer was extracted with EtOAc (×2). The combined organic layers were dried through a hydrophobic frit and concentrated in vacuo. The crude product was purified by column chromatography (eluting with 0 to 50% EtOAc in heptane) to provide the title compound (863 mg).

¹H NMR (400 MHz, MeOD) δ 7.80 (dd, J=8.3, 5.1 Hz, 1H), 7.35-7.26 (m, 2H), 5.66 (dd, J=8.0, 3.6 Hz, 1H), 3.21 (dd, J=18.9, 7.9 Hz, 1H), 2.66 (dd, J=18.9, 3.6 Hz, 1H).

Exchangeable proton not observed.

LCMS (Method 6): 1.69 min, 260.2 [M−H]⁻

### Synthesis of 2,2,2-trifluoro-N-(6′-fluorospiro[1,3-dithiolane-2,3′-indane]-1′-yl) acetamide

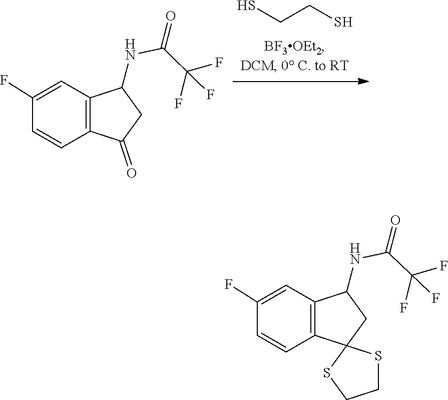

BF₃·OEt₂ (900 μL, 7.27 mmol) was added dropwise to a suspension of ethane-1,2-dithiol (230 μL, 2.73 mmol) and 2,2,2-trifluoro-N-(6-fluoro-3-oxo-indan-1-yl) acetamide (475 mg, 1.82 mmol) in DCM (10 mL) at 0° C., then the reaction was warmed to room temperature and stirred for 19 h. The reaction was quenched with sat. aq. NaHCO₃ and diluted with EtOAc. The layers were separated, and the aqueous layer was extracted with EtOAc. The combined organic layers were dried through a hydrophobic frit and concentrated in vacuo to provide the title compound (596 mg).

¹H NMR (400 MHz, CDCl₃) δ 7.62-7.55 (m, 1H), 7.14-7.05 (m, 1H), 7.05-6.98 (m, 1H), 6.76 (s, 1H), 5.55-5.46 (m, 1H), 3.62-3.54 (m, 2H), 3.53-3.44 (m, 2H), 3.16 (dd, J=13.9, 7.1 Hz, 1H), 2.64 (dd, J=14.0, 4.6 Hz, 1H)

LCMS (Method 6): 2.13 min, 336.0 [M−H]⁻

### Synthesis of 2,2,2-trifluoro-N-(3,3,6-trifluoroindan-1-yl) acetamide

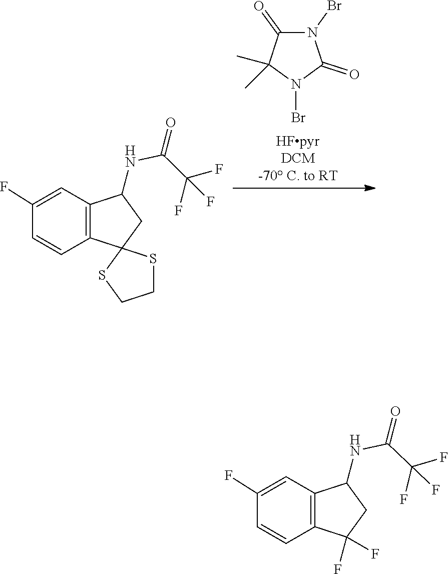 [SMILES: CC1(C)C(=O)N(Br)C(=O)N1Br ; O=C(NC1CC2(SCCS2)c2ccc(F)cc21)C(F)(F)F ; O=C(NC1CC(F)(F)c2ccc(F)cc21)C(F)(F)F]

HF.pyr (2.03 mL, 15.74 mmol) was added dropwise to a suspension of 1,3-dibromo-5,5-dimethyl-imidazolidine-2,4-dione (2 g, 7.00 mmol) in DCM (6 mL) at −70° C., and the reaction was stirred at −70° C. for 1.5 h. A solution of 2,2,2-trifluoro-N-(6′-fluorospiro[1,3-dithiolane-2,3′-indane]-1′-yl) acetamide (590 mg, 1.75 mmol) in DCM (4 mL) was added dropwise, and the reaction was stirred at −70° C. for 3 h then at room temperature for a further 1 h. The reaction mixture was quenched with 2 M aq. NaOH at 0° C. and diluted with DCM. The layers were separated, and the organic layer was washed with brine, dried through a hydrophobic frit, and concentrated in vacuo. The crude product was purified twice by column chromatography (eluting first with 0 to 80% EtOAc in heptane and then with 0 to 80% DCM in heptane) to provide the title compound (145 mg) as the impure product.

¹H NMR (400 MHz, CDCl₃) δ 7.64-7.57 (m, 1H), 7.21 (td, J=8.6, 2.3 Hz, 1H), 7.15-7.08 (m, 1H), 6.54 (s, 1H), 5.65-5.55 (m, 1H), 3.27-3.11 (m, 1H), 2.59-2.44 (m, 1H)

LCMS (Method 6): 2.00 min, 282.0 [M−H]⁻

### Synthesis of 3,3,6-trifluoroindan-1-amine

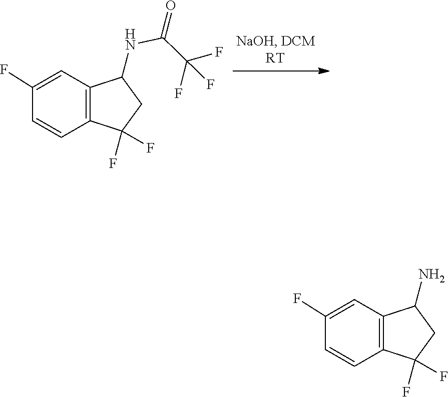 [SMILES: O=C(NC1CC(F)(F)c2ccc(F)cc21)C(F)(F)F ; NC1CC(F)(F)c2ccc(F)cc21]

4 M aq. NaOH (1.24 mL, 4.94 mmol) was added to a solution of 2,2,2-trifluoro-N-(3,3,6-trifluoroindan-1-yl) acetamide (140 mg, 0.49 mmol) in DCM (3 mL) and the reaction was stirred at room temperature for 24 h. The reaction mixture was diluted with DCM and water and the layers were separated. The aqueous layer was extracted with DCM (×2), and the combined organic layers were dried through a hydrophobic frit and concentrated in vacuo. The crude product was purified by column chromatography (eluting with 0 to 10% MeOH in DCM) to provide the title compound (62 mg).

¹H NMR (400 MHz, CDCl₃) δ 7.55-7.47 (m, 1H), 7.19-7.04 (m, 2H), 4.51-4.41 (m, 1H), 3.13-2.98 (m, 1H), 2.36-2.18 (m, 1H). Exchangeable protons not observed.

### Synthesis of Compound 237, 2-(methoxymethyl)-N7-(3,3,6-trifluoroindan-1-yl) pyrazolo[1,5-a]pyrimidine-3,7-dicarboxamide

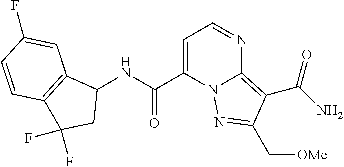 [SMILES: COCc1nn2c(C(=O)NC3CC(F)(F)c4ccc(F)cc43)ccnc2c1C(N)=O]

2-(Methoxymethyl)-N7-(3,3,6-trifluoroindan-1-yl)pyrazolo[1,5-a]pyrimidine-3,7-dicarboxamide (51 mg) was synthesised following a similar procedure to that described in General Method E.

¹H NMR (400 MHz, DMSO-d₆) δ 10.35 (d, J=7.6 Hz, 1H), 8.94 (d, J=4.4 Hz, 1H), 7.88-7.54 (m, 4H), 7.56-7.47 (m, 1H), 7.46-7.36 (m, 1H), 5.80-5.61 (m, 1H), 5.05-4.84 (m, 2H), 3.37-3.22 (m, 4H), 2.77-2.60 (m, 1H)

LCMS (Method 7): 2.47 min, 420.1 [M+H]⁺

### Synthesis of Compound 238, N7-[(3S)-6,7-difluoro-2,3-dihydrobenzofuran-3-yl]-2-(methoxymethyl)pyrazolo[1,5-a]pyrimidine-3,7-dicarboxamide

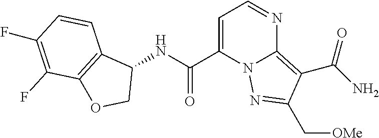 [SMILES: COCc1nn2c(C(=O)N[C@@H]3COc4c3ccc(F)c4F)ccnc2c1C(N)=O]

N7-[(3S)-6,7-difluoro-2,3-dihydrobenzofuran-3-yl]-2-(methoxymethyl)pyrazolo[1,5-a]pyrimidine-3,7-dicarboxamide (97 mg) was synthesised following a similar procedure to that described in General Method E.

¹H NMR (400 MHz, DMSO-d₆) δ 10.41 (d, J=6.8 Hz, 1H), 8.93 (d, J=4.4 Hz, 1H), 7.69 (d, J=4.4 Hz, 1H), 7.63 (s, 1H), 7.58 (s, 1H), 7.35-7.27 (m, 1H), 7.07-6.96 (m, 1H), 5.87-5.77 (m, 1H), 5.07-4.96 (m, 1H), 4.94 (d, J=2.8 Hz, 2H), 4.69 (dd, J=10.0, 4.3 Hz, 1H), 3.30 (s, 3H)

LCMS (Method 7): 2.36 min, 404.1 [M+H]⁺

### Synthesis of 7′-fluorospiro[1,3-dithiolane-2,1′-tetralin]

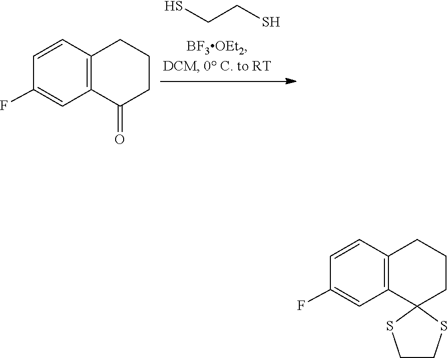

BF₃·OEt₂ (6 mL, 48.73 mmol) was added to a solution of ethane-1,2-dithiol (1.54 mL, 18.27 mmol) and 7-fluorotetralin-1-one (2 g, 12.18 mmol) in DCM (50 mL) at 0° C., then the reaction was warmed to room temperature and stirred for 16 h. The reaction mixture was quenched with sat. aq. NaHCO₃ and diluted with DCM. The layers were separated, and the aqueous layer was extracted with DCM. The combined organic layers were dried over Na₂SO₄ and concentrated in vacuo to provide the title compound (3.08 g).

¹H NMR (400 MHz, CDCl₃) δ 7.62 (dd, J=10.8, 2.7 Hz, 1H), 6.98-6.90 (m, 1H), 6.87-6.77 (m, 1H), 3.64-3.52 (m, 2H), 3.52-3.40 (m, 2H), 2.76-2.73 (m, 2H), 2.42-2.33 (m, 2H), 2.04-1.94 (m, 2H)

LCMS (Method 7): 3.41 mins, 241.2 [M+H]⁺

### Synthesis of 1,1,7-trifluorotetralin

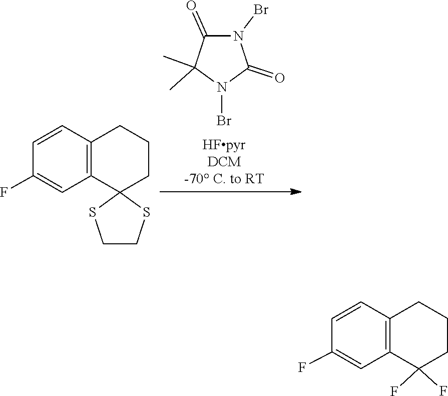

HF.pyr (4.82 mL, 37.45 mmol) was added dropwise to a solution of 1,3-dibromo-5,5-dimethyl-imidazolidine-2,4-dione (4.76 g, 16.64 mmol) in DCM (7 mL) at −70° C., and the reaction was stirred at −70° C. for 30 min. A solution of 7′-fluorospiro[1,3-dithiolane-2,1′-tetralin] (1 g, 4.16 mmol) in DCM (2 mL) was added dropwise, and the reaction was stirred at −70° C. for 3 h then at room temperature for a further 1 h. The reaction mixture was quenched with 2 M aq. NaOH at 0° C. and diluted with DCM. The layers were separated, and the organic layer was washed with sat. aq. NaHSO₃, dried over Na₂SO₄, and concentrated in vacuo. The crude product was purified by column chromatography (eluting with 0 to 10% EtOAc in heptane) to provide the title compound (263 mg).

¹H NMR (400 MHz, CDCl₃) δ 7.35 (dd, J=9.2, 2.7 Hz, 1H), 7.18-7.00 (m, 2H), 2.84-2.75 (m, 2H), 2.34-2.18 (m, 2H), 2.05-1.94 (m, 2H)

LCMS (Method 7): 3.04 min (product does not ionise)

### Synthesis of 4,4,6-trifluorotetralin-1-one

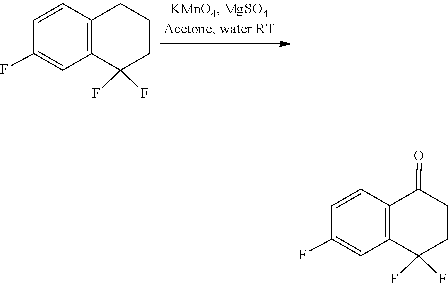 [SMILES: Fc1ccc2c(c1)C(F)(F)CCC2 ; O=C1CCC(F)(F)c2cc(F)ccc21]

MgSO₄ (336 mg, 2.79 mmol) and KMnO₄ (660 mg, 4.19 mmol) were added sequentially to a solution of 1,1,7-trifluorotetralin (260 mg, 1.4 mmol) in acetone (3 mL) and water (3 mL), and the reaction was stirred at room temperature for 3 h. EtOH (10 mL) was added, and the reaction mixture was stirred for a further 1 h, then filtered and concentrated in vacuo. The residue was diluted with DCM and water, and the layers were separated. The organic layer was dried over Na₂SO₄ and concentrated in vacuo. The crude product was purified by column chromatography (eluting with 0 to 10% EtOAc in heptane) to provide the title compound (131 mg).

¹H NMR (400 MHz, CDCl₃) δ 8.14-8.06 (m, 1H), 7.49-7.41 (m, 1H), 7.33-7.23 (m, 1H), 2.90 (dd, J=7.3, 6.2 Hz, 2H), 2.73-2.59 (m, 2H)

LCMS (Method 7): 3.04 min (product does not ionise)

### Synthesis of 4,4,6-trifluorotetralin-1-one oxime

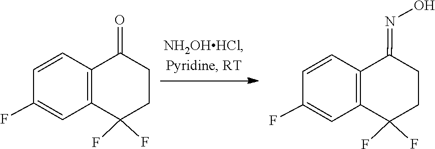 [SMILES: O=C1CCC(F)(F)c2cc(F)ccc21 ; O/N=C1\CCC(F)(F)c2cc(F)ccc21]

Hydroxylamine hydrochloride (48 mg, 0.70 mmol) was added to a solution of 4,4,6-trifluorotetralin-1-one (127 mg, 0.63 mmol) in pyridine (1 mL) and the reaction was stirred at room temperature for 5 h. The reaction mixture was diluted with EtOAc and acidified to pH 2 with 5 M aq. HCl. The layers were separated, and the organic layer was washed successively with 1 M aq. HCl, 10% aq. CuSO₄, water, and brine, dried over Na₂SO₄, and concentrated in vacuo to provide the title compound (122 mg).

¹H NMR (400 MHz, DMSO-d₆) δ 11.67 (s, 1H), 8.07-7.98 (m, 1H), 7.50-7.37 (m, 2H), 2.85 (t, J=7.0 Hz, 2H), 2.48-2.33 (m, 2H) LCMS (Method 7): 2.51 min (product does not ionise)

### Synthesis of 4,4,6-trifluorotetralin-1-amine

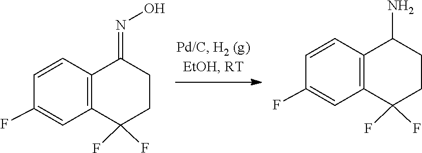 [SMILES: O/N=C1\CCC(F)(F)c2cc(F)ccc21 ; NC1CCC(F)(F)c2cc(F)ccc21]

10% Pd/C (25 mg, 0.23 mmol) was added to a solution of 4,4,6-trifluorotetralin-1-one oxime (122 mg, 0.57 mmol) in EtOH (5 mL) under an atmosphere of argon. A balloon of hydrogen was attached to the flask and the reaction was stirred at room temperature for 18 h. The reaction mixture was filtered through a short pad of Dicalite® and concentrated in vacuo. The crude product was purified by SCX chromatography (eluting with 2 M NH₃ in MeOH) and then by column chromatography (eluting with 0 to 3% (2 M NH₃ in MeOH) in DCM) to provide the title compound (36 mg).

¹H NMR (400 MHz, CDCl₃) δ 7.56-7.47 (m, 1H), 7.37-7.29 (m, 1H), 7.19-7.09 (m, 1H), 4.04-3.95 (m, 1H), 2.60-2.43 (m, 1H), 2.31-2.12 (m, 2H), 1.91-1.77 (m, 1H), 1.54 (s, 2H)

### Synthesis of Compound 239, 2-(methoxymethyl)-N7-(4,4,6-trifluorotetralin-1-yl)pyrazolo[1,5-a]pyrimidine-3,7-dicarboxamide

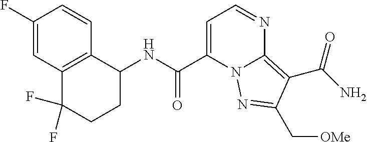 [SMILES: COCc1nn2c(C(=O)NC3CCC(F)(F)c4cc(F)ccc43)ccnc2c1C(N)=O]

2-(Methoxymethyl)-N7-(4,4,6-trifluorotetralin-1-yl)pyrazolo[1,5-a]pyrimidine-3,7-dicarboxamide (29 mg) was synthesised following a similar procedure to that described in General Method E.

¹H NMR (400 MHz, DMSO-d₆) δ 10.24 (d, J=8.3 Hz, 1H), 8.93 (d, J=4.4 Hz, 1H), 7.75 (dd, J=8.7, 5.4 Hz, 1H), 7.71-7.54 (m, 3H), 7.52-7.37 (m, 2H), 5.45-5.39 (m, 1H), 4.95 (s, 2H), 3.32 (s, 3H), 2.59-2.51 (m, 2H), 2.34-2.26 (m, 1H), 2.15-2.01 (m, 1H)

LCMS (Method 7): 2.57 min, 434.2 [M+H]⁺

### Synthesis of Compound 94, N7-(trans-3-fluoro-3-methyl-cyclobutyl)-2-(methoxymethyl)pyrazolo[1,5-a]pyrimidine-3,7-dicarboxamide (General Method F)

### Synthesis of 3-cyano-2-(methoxymethyl)pyrazolo[1,5-a]pyrimidine-7-carboxylic Acid

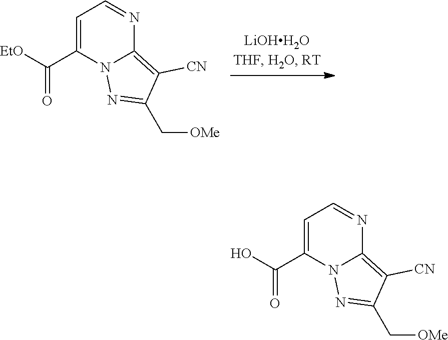 [SMILES: CCOC(=O)c1ccnc2c(C#N)c(COC)nn12 ; COCc1nn2c(C(=O)O)ccnc2c1C#N]

A solution of LiOH·H₂O (35 mg, 0.85 mmol) in water (1 mL) was added to a solution of ethyl 3-cyano-2-(methoxymethyl)pyrazolo[1,5-a]pyrimidine-7-carboxylate (200 mg, 0.77 mmol) in THF (1 mL) and the reaction was stirred at room temperature for 3 h. The reaction mixture was concentrated in vacuo and the aqueous residue was acidified with 5 M aq. HCl. The suspension was diluted with water, and the precipitate was collected by filtration, washed with water, and dried to provide the title compound (146 mg).

¹H NMR (400 MHz, DMSO-d₆) δ 8.94 (d, J=4.3 Hz, 1H), 7.66 (d, J=4.3 Hz, 1H), 4.74 (s, 2H), 3.38 (s, 3H). Exchangeable proton not observed.

LCMS (Method 1): 1.23 min, 233.2 [M+H]⁺

### Synthesis of 3-cyano-N-(trans-3-fluoro-3-methyl-cyclobutyl)-2-(methoxymethyl)pyrazolo[1,5-a]pyrimidine-7-carboxamide

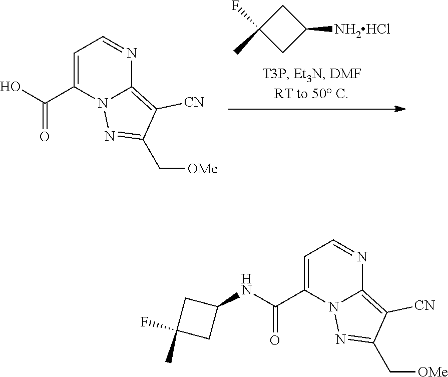

Et₃N (108 μL, 0.78 mmol) was added to a solution of trans-3-fluoro-3-methyl-cyclobutanamine hydrochloride (30 mg, 0.21 mmol) and 3-cyano-2-(methoxymethyl)pyrazolo[1,5-a]pyrimidine-7-carboxylic acid (45 mg, 0.19 mmol) in DMF (500 μL) and the mixture was stirred at room temperature for 5 min. 1-Propanephosphonic anhydride (50 wt % in DMF) (173 μL, 0.29 mmol) was added and the reaction was stirred at room temperature for 18 h. Additional portions of Et₃N (108 μL, 0.78 mmol) and 1-propanephosphonic anhydride (50 wt % in DMF) (173 μL, 0.29 mmol) were added and the reaction was stirred at room temperature for 6 h and then heated at 50° C. for 2 h. The reaction mixture was cooled to room temperature and diluted with 1 M aq. HCl and EtOAc. The layers were separated, and the organic layer was washed with brine, dried over Na₂SO₄, and concentrated in vacuo. The crude product was purified by column chromatography (eluting with 0 to 70% EtOAc in heptane) to provide the title compound (19 mg).

¹H NMR (400 MHz, CDCl₃) δ 10.20 (s, 1H), 8.91 (d, J=4.3 Hz, 1H), 8.00 (d, J=4.3 Hz, 1H), 4.84 (s, 2H), 4.75-4.65 (m, 1H), 3.58 (s, 3H), 2.98-2.86 (m, 2H), 2.34-2.24 (m, 2H), 1.60 (d, J=22.0 Hz, 3H)

LCMS (Method 1): 1.65 min, 318.2 [M+H]⁺

### Synthesis of Compound 94, N7-(trans-3-fluoro-3-methyl-cyclobutyl)-2-(methoxymethyl)pyrazolo[1,5-a]pyrimidine-3,7-dicarboxamide

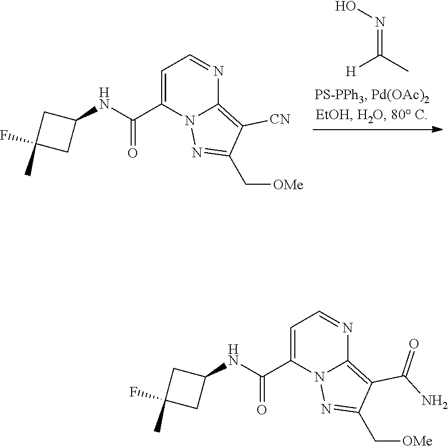

3-Cyano-N-(trans-3-fluoro-3-methyl-cyclobutyl)-2-(methoxymethyl)pyrazolo[1,5-a]pyrimidine-7-carboxamide (19 mg, 0.06 mmol), acetaldehyde oxime (14 μL, 0.23 mmol), and PS-PPh₃ (1.4-2 mmol/g; 15 mg, 0.02-0.03 mmol) were suspended in EtOH (440 μL) and water (110 μL) and the reaction was heated at 80° C. for 3 hours. The reaction mixture was cooled to room temperature and concentrated in vacuo. The crude product was purified by column chromatography (eluting with 0 to 4% MeOH in DCM) and preparative HPLC (basic method) to provide the title compound (6.9 mg).

¹H NMR (400 MHz, DMSO-d₆) δ 10.15 (d, J=6.1 Hz, 1H), 8.92 (d, J=4.4 Hz, 1H), 7.68 (br. s, 1H), 7.65 (d, J=4.3 Hz, 1H), 7.60 (br. s, 1H), 5.00 (s, 2H), 4.57-4.48 (m, 1H), 3.43 (s, 3H), 2.84-2.71 (m, 2H), 2.33-2.20 (m, 2H), 1.55 (d, J=22.4 Hz, 3H)

LCMS (Method 2): 1.91 min, 336.2 [M+H]⁺

### Synthesis of Compound 134, 2-cyclopropyl-N7-(5,6-dihydro-4H-cyclopenta[c]thiophen-5-yl)pyrazolo[1,5-a]pyrimidine-3,7-dicarboxamide

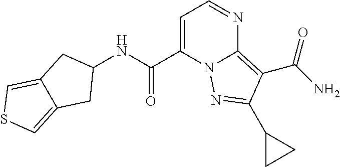 [SMILES: NC(=O)c1c(C2CC2)nn2c(C(=O)NC3Cc4cscc4C3)ccnc12]

2-Cyclopropyl-N7-(5,6-dihydro-4H-cyclopenta[c]thiophen-5-yl)pyrazolo[1,5-a]pyrimidine-3,7-dicarboxamide (8.1 mg) was synthesised following a similar procedure to that described in General Method F.

¹H NMR (400 MHz, CDCl₃) δ 10.70 (d, J=7.6 Hz, 1H), 8.70 (d, J=4.4 Hz, 1H), 8.15 (s, 1H), 7.86 (d, J=4.4 Hz, 1H), 6.96 (s, 2H), 5.61 (s, 1H), 5.17 (s, 1H), 3.31-3.20 (m, 3H), 2.77 (dd, J=15.9, 4.2 Hz, 2H), 1.19-1.11 (m, 2H), 0.87-0.79 (m, 2H)

LCMS (Method 6): 1.88 min, 368.2 [M+H]⁺

### Synthesis of Compound 135, 2-cyclopropyl-N7-(4,4-difluorocyclohexyl)pyrazolo[1,5-a]pyrimidine-3,7-dicarboxamide

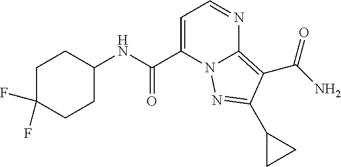 [SMILES: NC(=O)c1c(C2CC2)nn2c(C(=O)NC3CCC(F)(F)CC3)ccnc12]

2-Cyclopropyl-N7-(4,4-difluorocyclohexyl)pyrazolo[1,5-a]pyrimidine-3,7-dicarboxamide (24 mg) was synthesised following a similar procedure to that described in General Method F.

¹H NMR (400 MHz, CDCl₃) δ 10.40 (d, J=7.3 Hz, 1H), 8.73 (d, J=4.4 Hz, 1H), 8.18 (s, 1H), 7.86 (d, J=4.4 Hz, 1H), 5.66 (s, 1H), 4.22-4.18 (m, 1H), 3.43-3.33 (m, 1H), 2.23-2.10 (m, 4H), 2.09-1.95 (m, 2H), 1.85-1.74 (m, 2H), 1.33-1.24 (m, 2H), 1.12-1.04 (m, 2H)

LCMS (Method 7): 2.46 min, 364.2 [M+H]⁺

### Synthesis of Compound 136, 2-cyclopropyl-N7-(3,3-difluorocyclobutyl)pyrazolo[1,5-a]pyrimidine-3,7-dicarboxamide

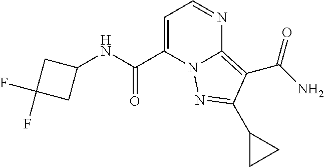 [SMILES: NC(=O)c1c(C2CC2)nn2c(C(=O)NC3CC(F)(F)C3)ccnc12]

2-Cyclopropyl-N7-(3,3-difluorocyclobutyl)pyrazolo[1,5-a]pyrimidine-3,7-dicarboxamide (42 mg) was synthesised following a similar procedure to that described in General Method F.

¹H NMR (400 MHz, DMSO-d₆) δ 10.10 (s, 1H), 8.85 (d, J=4.5 Hz, 1H), 7.77 (s, 1H), 7.60 (d, J=4.4 Hz, 1H), 7.55 (s, 1H), 4.37 (s, 1H), 3.26-3.07 (m, 3H), 2.76-2.61 (m, 2H), 1.18-1.11 (m, 2H), 1.11-1.05 (m, 2H)

LCMS (Method 7): 2.40 min, 336.2 [M+H]⁺

### Synthesis of Compound 137, 2-propyl-N7-[cis-3-(trifluoromethoxy)cyclobutyl]pyrazolo[1,5-a]pyrimidine-3,7-dicarboxamide

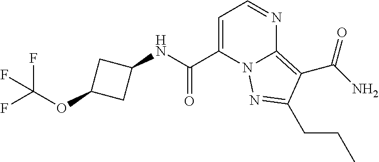 [SMILES: CCCc1nn2c(C(=O)N[C@H]3C[C@@H](OC(F)(F)F)C3)ccnc2c1C(N)=O]

2-Propyl-N7-[cis-3-(trifluoromethoxy)cyclobutyl]pyrazolo[1,5-a]pyrimidine-3,7-dicarboxamide (84 mg) was synthesised following a similar procedure to that described in General Method F.

¹H NMR (400 MHz, DMSO-d₆) δ 10.10 (d, J=7.4 Hz, 1H), 8.87 (d, J=4.3 Hz, 1H), 7.73 (d, J=2.7 Hz, 1H), 7.59 (d, J=4.4 Hz, 1H), 7.49 (d, J=2.5 Hz, 1H), 4.77-4.66 (m, 1H), 4.28-4.13 (m, 1H), 3.20-3.11 (m, 2H), 2.96-2.85 (m, 2H), 2.38-2.26 (m, 2H), 1.85-1.72 (m, 2H), 0.99 (t, J=7.4 Hz, 3H)

LCMS (Method 7) 2.83 min, 386.2 [M+H]⁺

### Synthesis of Compound 138, N7-(7-bicyclo[4.2.0]octa-1,3,5-trienyl)-2-cyclopropyl-pyrazolo[1,5-a]pyrimidine-3,7-dicarboxamide

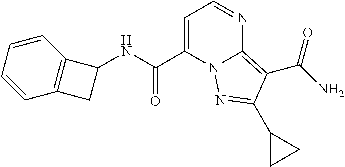 [SMILES: NC(=O)c1c(C2CC2)nn2c(C(=O)NC3Cc4ccccc43)ccnc12]

N7-(7-bicyclo[4.2.0]octa-1,3,5-trienyl)-2-cyclopropyl-pyrazolo[1,5-a]pyrimidine-3,7-dicarboxamide (24 mg) was synthesised following a similar procedure to that described in General Method F.

¹H NMR (400 MHz, DMSO-d₆) δ 10.40 (d, J=6.8 Hz, 1H), 8.86 (d, J=4.4 Hz, 1H), 7.76 (d, J=2.8 Hz, 1H), 7.65 (d, J=4.4 Hz, 1H), 7.57-7.50 (m, 1H), 7.40-7.25 (m, 3H), 7.28-7.21 (m, 1H), 5.55-5.47 (m, 1H), 3.77 (dd, J=14.3, 5.0 Hz, 1H), 3.21-3.10 (m, 2H), 1.17-0.99 (m, 2H), 0.97-0.81 (m, 2H)

LCMS (Method 7): 2.70 min, 348.2 [M+H]⁺

### Synthesis of Compound 139, N7-indan-2-yl-2-propyl-pyrazolo[1,5-a]pyrimidine-3,7-dicarboxamide

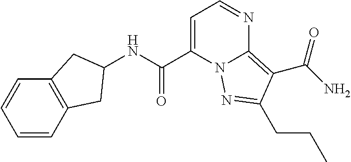 [SMILES: CCCc1nn2c(C(=O)NC3Cc4ccccc4C3)ccnc2c1C(N)=O]

N7-indan-2-yl-2-propyl-pyrazolo[1,5-a]pyrimidine-3,7-dicarboxamide (67 mg) was synthesised following a similar procedure to that described in General Method F.

¹H NMR (400 MHz, DMSO-d₆) δ 10.37 (d, J=7.4 Hz, 1H), 8.87 (d, J=4.4 Hz, 1H), 7.71 (s, 1H), 7.68 (d, J=4.4 Hz, 1H), 7.47 (s, 1H), 7.35-7.26 (m, 2H), 7.22-7.18 (m, 2H), 4.85-4.77 (m, 1H), 3.37 (dd, J=16.1, 6.9 Hz, 2H), 3.07-2.93 (m, 4H), 1.61-1.52 (m, 2H), 0.89 (t, J=7.4 Hz, 3H)

LCMS (Method 7): 2.88 min, 364.2 [M+H]⁺

### Synthesis of Compound 140, 2-cyclopropyl-N7-[(4-fluorophenyl)methyl]pyrazolo[1,5-a]pyrimidine-3,7-dicarboxamide

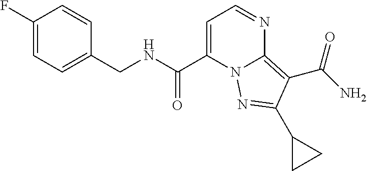 [SMILES: NC(=O)c1c(C2CC2)nn2c(C(=O)NCc3ccc(F)cc3)ccnc12]

2-Cyclopropyl-N7-[(4-fluorophenyl)methyl]pyrazolo[1,5-a]pyrimidine-3,7-dicarboxamide (49 mg) was synthesised following a similar procedure to that described in General Method F.

¹H NMR (400 MHz, DMSO-d₆): δ 10.05 (t, J=5.7 Hz, 1H), 8.85 (d, J=4.4 Hz, 1H), 7.77 (br. s, 1H), 7.62 (d, J=4.4 Hz, 1H), 7.54 (br. s, 1H), 7.52-7.47 (m, 2H), 7.26-7.16 (m, 2H), 4.66-4.60 (m, 2H), 3.23-3.15 (m, 1H), 1.12-1.06 (m, 2H), 1.01-0.94 (m, 2H)

LCMS (Method 7): 2.48 min, 354.2 [M+H]⁺

### Synthesis of Sodium 1,1-dicyano-3-ethoxy-3-oxo-prop-1-en-2-olate

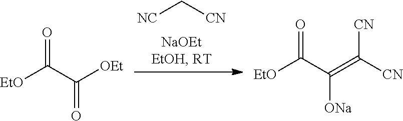

NaOEt (21 wt % in EtOH) (9.4 mL, 25.19 mmol) was added dropwise to a solution of propanedinitrile (1.64 g, 24.84 mmol) and diethyl oxalate (3.37 mL, 24.84 mmol) in EtOH (40 mL) and the reaction was stirred at room temperature for 1 h then concentrated in vacuo. The residue was dissolved in EtOAc (75 mL) and the solution was stirred at room temperature for 1 h. The solvent was decanted, filtered through a short pad of Dicalite®, and concentrated in vacuo. The residue was suspended in EtOH and Et₂O, and the mixture was sonicated for 5 min. The solvent was decanted, and the residual gum was washed with Et₂O and dried to provide the title compound (2.27 g). The crude product was taken forward to the next step without purification.

### Synthesis of ethyl 3,3-dicyano-2-methoxy-prop-2-enoate

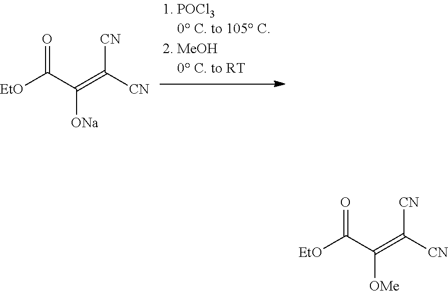

Sodium 1,1-dicyano-3-ethoxy-3-oxo-prop-1-en-2-olate (10 g, 53.16 mmol) was added to POCl₃ (30 mL, 321.85 mmol) at 0° C. The mixture was warmed to room temperature and stirred until the solid was in solution. The reaction was heated at 105° C. for 45 min then cooled to room temperature. The reaction mixture was concentrated in vacuo and the residue was cooled to 0° C. MeOH (200 mL) was added slowly and the reaction was stirred at room temperature for 15 min. The reaction mixture was concentrated in vacuo and the residue was dissolved in a 1:2.5 mixture of EtOAc:heptane. The solution was filtered through a plug of silica gel and concentrated in vacuo to provide the title compound (4.33 g). The crude product was taken forward to the next step without further purification.

### Synthesis of ethyl 5-amino-4-cyano-1H-pyrazole-3-carboxylate

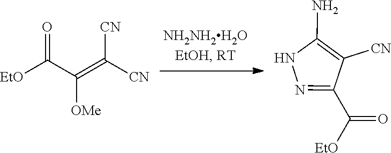 [SMILES: CCOC(=O)C(OC)=C(C#N)C#N ; CCOC(=O)c1n[nH]c(N)c1C#N]

A solution of ethyl 3,3-dicyano-2-methoxy-prop-2-enoate (4.47 g, 24.81 mmol) in EtOH (20 mL) was added to a solution of hydrazine monohydrate (1.2 mL, 24.81 mmol) in EtOH (150 mL) and the reaction was stirred at room temperature for 1.25 h. The resulting precipitate was collected by filtration, washed with cold EtOH, and dried to provide the crude product. The filtrate was concentrated in vacuo, and the residue was suspended in EtOAc and sonicated for 5 min. The resulting precipitate was collected by filtration, washed with EtOAc, and dried to provide a second batch of the crude product. The batches were combined to provide the title compound (2.38 g).

¹H NMR (400 MHz, DMSO-d₆) δ 12.68 (s, 1H), 6.60 (s, 2H), 4.27 (q, J=7.1 Hz, 2H), 1.28 (t, J=7.1 Hz, 3H)

LCMS (Method 6): 1.21 min, 181.2 [M+H]⁺

### Synthesis of 5-amino-3-(hydroxymethyl)-1H-pyrazole-4-carbonitrile

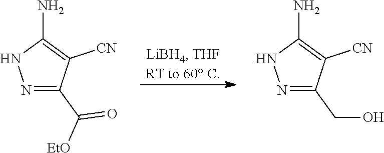 [SMILES: CCOC(=O)c1n[nH]c(N)c1C#N ; N#Cc1c(CO)n[nH]c1N]

LiBH₄ (2 M in THF) (10.3 mL, 20.65 mmol) was added slowly to a solution of ethyl 5-amino-4-cyano-1H-pyrazole-3-carboxylate (1.5 g, 8.33 mmol) in THF (21 mL) and the reaction was heated at 60° C. for 18 h. The reaction mixture was cooled to room temperature and diluted with MeOH. The mixture was stirred at room temperature for 1 h then concentrated in vacuo. The crude product was purified by column chromatography (eluting with 0 to 20% MeOH in DCM) to provide the title compound (863 mg).

LCMS (Method 6): 0.16 min, 139.3 [M+H]⁺

### Synthesis of ethyl 3-cyano-2-(hydroxymethyl)pyrazolo[1,5-a]pyrimidine-7-carboxylate

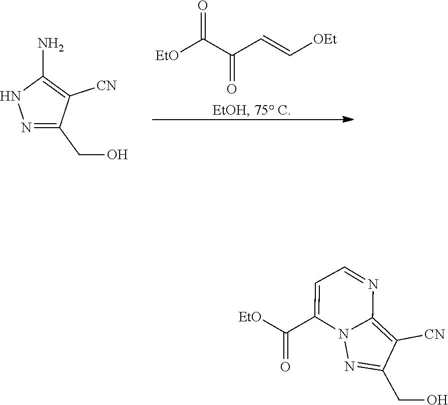 [SMILES: N#Cc1c(CO)n[nH]c1N ; CCO/C=C/C(=O)C(=O)OCC ; CCOC(=O)c1ccnc2c(C#N)c(CO)nn12]

5-Amino-3-(hydroxymethyl)-1H-pylole-4-carbonitrile (863 mg, 6.25 mmol) and ethyl (E)-4-ethoxy-2-oxo-but-3-enoate (1.61 g, 9.37 mmol) were dissolved in EtOH (14.7 mL) and the reaction was heated at 75° C. in a microwave reactor for 2 h. The reaction mixture was cooled to room temperature and concentrated in vacuo. The crude product was purified by column chromatography (eluting with 0 to 100% EtOAc in heptane) to provide the title compound (736 mg).

¹H NMR (400 MHz, CDCl₃) δ 8.82 (d, J=4.2 Hz, 1H), 7.51 (d, J=4.2 Hz, 1H), 5.09 (s, 2H), 4.58 (q, J=7.2 Hz, 2H), 1.48 (t, J=7.1 Hz, 3H). Exchangeable proton not observed.

LCMS (Method 6): 1.42 min (split peak), 247.2 [M+H]⁺

### Synthesis of 3-cyano-2-(hydroxymethyl)pyrazolo[1,5-a]pyrimidine-7-carboxylic Acid

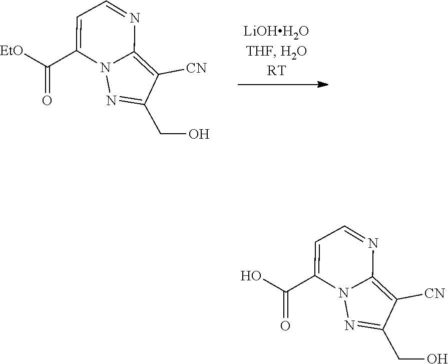 [SMILES: CCOC(=O)c1ccnc2c(C#N)c(CO)nn12 ; N#Cc1c(CO)nn2c(C(=O)O)ccnc12]

LiOH·H₂O (51 mg, 1.22 mmol) was added to a solution of ethyl 3-cyano-2-(hydroxymethyl)pyrazolo[1,5-a]pyrimidine-7-carboxylate (251 mg, 1.02 mmol) in THF (3.5 mL) and water (860 μL) and the reaction was stirred at room temperature for 18 h. The reaction mixture was acidified to pH 1 with 1 M aq. HCl and the resulting suspension was concentrated in vacuo to provide the title compound (260 mg).

¹H NMR (400 MHz, DMSO-d₆) δ 8.73 (d, J=4.4 Hz, 1H), 7.17 (d, J=4.4 Hz, 1H), 5.74 (s, 1H), 4.70 (s, 2H). Additional exchangeable proton not observed.

LCMS (Method 6): 1.02 min, 219.2 [M+H]⁺

### Synthesis of 3-cyano-2-(hydroxymethyl)-N-[cis-3-(trifluoromethoxy)cyclobutyl]pyrazolo[1,5-a]pyrimidine-7-carboxamide

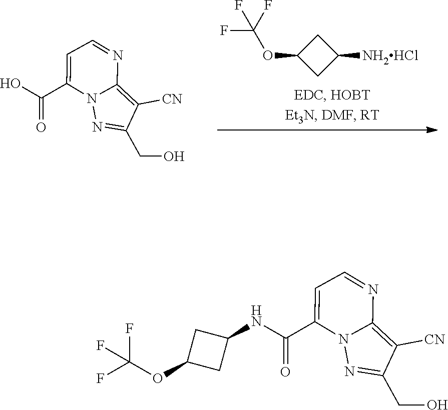

Cis-3-(trifluoromethoxy)cyclobutanamine hydrochloride (226 mg, 1.18 mmol), HOBt (266 mg, 1.97 mmol), EDC (377 mg, 1.97 mmol), and Et₃N (549 μL, 3.94 mmol) were added to a suspension of 3-cyano-2-(hydroxymethyl)pyrazolo[1,5-a]pyrimidine-7-carboxylic acid (179 mg, 0.66 mmol) in DMF (2.6 mL) and the reaction was stirred at room temperature for 21 h. The reaction mixture was diluted with water and DCM and the layers were separated. The aqueous layer was extracted with DCM C 2), and the combined organic layers were dried through a hydrophobic frit and concentrated in vacuo. The crude product was purified by column chromatography (eluting with 0 to 80% EtOAc in heptane) to provide the title compound (193 mg).

¹H NMR (400 MHz, DMSO-d₆) δ 9.77 (d, J=5.8 Hz, 1H), 8.95 (d, J=4.4 Hz, 1H), 7.66 (d, J=4.4 Hz, 1H), 5.85 (t, J=5.7 Hz, 1H), 4.82 (d, J=5.1 Hz, 2H), 4.77-4.63 (m, 1H), 4.24-4.12 (m, 1H), 2.91-2.80 (m, 2H), 2.41-2.30 (m, 2H)

LCMS (Method 6) 1.79 min, 356.2 [M+H]⁺

### Synthesis of Compound 141, 2-(hydroxymethyl)-N7-[cis-3-(trifluoromethoxy)cyclobutyl]pyrazolo[1,5-a]pyrimidine-3,7-dicarboxamide

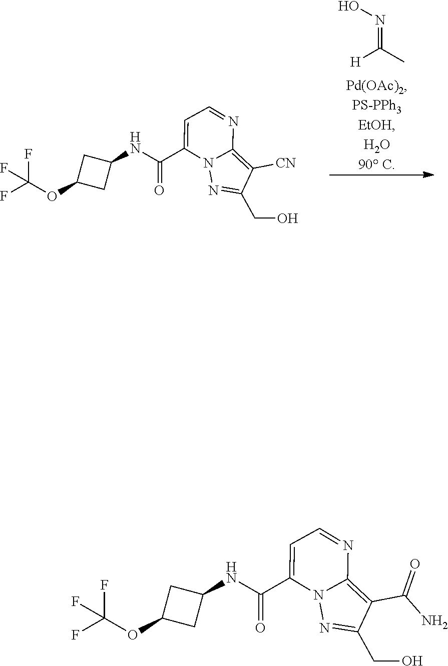

3-Cyano-2-(hydroxymethyl)-N-[cis-3-(trifluoromethoxy)cyclobutyl]pyrazolo[1,5-a]pyrimidine-7-carboxamide (188 mg, 0.53 mmol), acetaldehyde oxime (129 μL, 2.12 mmol), and PS-PPh₃ (1.4-2 mmol/g) (114 mg, 0.16-0.23 mmol) were suspended in EtOH (4 mL) and water (880 μL) and the mixture was degassed by sparging with a stream of argon for 10 min. Pd(OAc)₂ (18 mg, 0.08 mmol) was added and the mixture was sparged with argon for a further 10 min. The reaction was heated at 90° C. for 3.5 h. The reaction mixture was cooled to room temperature, diluted with DCM, and filtered through a short pad of Dicalite®. The filtrate was concentrated in vacuo, and the crude product was purified by column chromatography (eluting with 0 to 5% MeOH in DCM) to provide the title compound (145 mg).

¹H NMR (400 MHz, DMSO-d₆) δ 10.11 (d, J=7.1 Hz, 1H), 8.92 (d, J=4.4 Hz, 1H), 7.83 (s, 2H), 7.66 (d, J=4.4 Hz, 1H), 5.88 (t, J=6.4 Hz, 1H), 4.95 (d, J=5.4 Hz, 2H), 4.70 (p, J=7.3 Hz, 1H), 4.28-4.14 (m, 1H), 2.92-2.81 (m, 2H), 2.47-2.35 (m, 2H)

LCMS (Method 7): 2.26 min, 374.2 [M+H]⁺

### Synthesis of Compound 202, 2-(ethoxymethyl)-N7-indan-2-yl-pyrazolo[1,5-a]pyrimidine-3,7-dicarboxamide

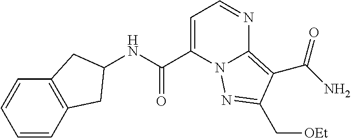 [SMILES: CCOCc1nn2c(C(=O)NC3Cc4ccccc4C3)ccnc2c1C(N)=O]

2-(Ethoxymethyl)-N7-indan-2-yl-pyrazolo[1,5-a]pyrimidine-3,7-dicarboxamide (40 mg) was synthesised following a similar procedure to that described in General Method F.

¹H NMR (400 MHz, DMSO-d₆) δ 10.22 (d, J=6.9 Hz, 1H), 8.91 (d, J=4.4 Hz, 1H), 7.69 (d, J=4.4 Hz, 1H), 7.68 (s, 1H), 7.57 (s, 1H), 7.29-7.26 (m, 2H), 7.22-7.15 (m, 2H), 4.96 (s, 2H), 4.84-4.76 (m, 1H), 3.55 (q, J=7.0 Hz, 2H), 3.41 (dd, J=16.2, 7.4 Hz, 2H), 2.97 (dd, J=16.2, 4.8 Hz, 2H), 1.11 (t, J=7.0 Hz, 3H)

LCMS (Method 7): 2.54 min, 380.0 [M+H]⁺

### Synthesis of Compound 204, 2-(hydroxymethyl)-N7-indan-2-yl-pyrazolo[1,5-a]pyrimidine-3,7-dicarboxamide

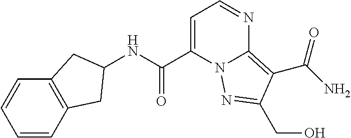 [SMILES: NC(=O)c1c(CO)nn2c(C(=O)NC3Cc4ccccc4C3)ccnc12]

2-(Hydroxymethyl)-N7-indan-2-yl-pyrazolo[1,5-a]pyrimidine-3,7-dicarboxamide (14 mg) was synthesised following a similar procedure to that described in General Method F.

¹H NMR (400 MHz, DMSO-d₆) δ 10.16 (d, J=7.1 Hz, 1H), 8.92 (d, J=4.4 Hz, 1H), 7.84 (s, 2H), 7.68 (d, J=4.4 Hz, 1H), 7.31-7.25 (m, 2H), 7.21-7.16 (m, 2H), 5.84 (s, 1H), 4.85 (s, 2H), 4.83-4.75 (m, 1H), 3.37 (dd, J=16.0, 7.6 Hz, 2H), 3.02 (dd, J=16.0, 5.8 Hz, 2H)

LCMS (Method 7): 2.27 min, 352.2 [M+H]⁺

### Synthesis of Compound 205, 2-(ethoxymethyl)-N7-[cis-3-(trifluoromethoxy)cyclobutyl]pyrazolo[1,5-a]pyrimidine-3,7-dicarboxamide

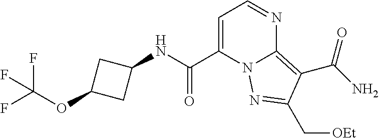 [SMILES: CCOCc1nn2c(C(=O)N[C@H]3C[C@@H](OC(F)(F)F)C3)ccnc2c1C(N)=O]

2-(Ethoxymethyl)-N7-[cis-3-(trifluoromethoxy)cyclobutyl]pyrazolo[1,5-a]pyrimidine-3,7-dicarboxamide (75 mg) was synthesised following a similar procedure to that described in General Method F.

¹H NMR (400 MHz, DMSO-d₆) δ 10.21 (d, J=7.4 Hz, 1H), 8.91 (d, J=4.4 Hz, 1H), 7.78-7.55 (m, 3H), 5.05 (s, 2H), 4.77-4.68 (m, 1H), 4.28-4.17 (m, 1H), 3.67 (q, J=7.0 Hz, 2H), 2.97-2.87 (m, 2H), 2.35-2.24 (m, 2H), 1.19 (t, J=7.0 Hz, 3H)

LCMS (Method 7): 2.53 min, 402.2 [M+H]⁺

### Synthesis of ethyl-4-ethoxy-2-oxo-pent-3-enoate

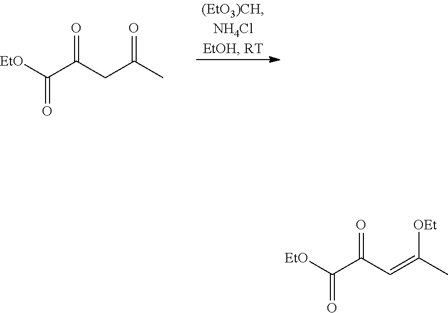

Ethyl 2,4-dioxopentanoate (2 g, 12.65 mmol), triethylorthoformate (2.52 mL, 15.18 mmol), and NH₄Cl (203 mg, 3.79 mmol) were suspended in EtOH (24 mL), and the reaction was stirred at room temperature for 21 h then concentrated in vacuo. The residue was dissolved in TBME, and the resulting suspension was filtered and concentrated in vacuo. The crude product was purified by vacuum distillation at 17 mbar to provide the title compound (1.78 g).

¹H NMR (400 MHz, CDCl₃) δ 6.21 (s, 1H), 4.30 (q, J=7.1 Hz, 2H), 4.00 (q, J=7.0 Hz, 2H), 2.38 (s, 3H), 1.39 (t, J=6.9 Hz, 3H), 1.35 (t, J=7.2 Hz, 3H)

LCMS (Method 6): 1.83 min, 187.2 [M+H]⁺

### Synthesis of Compound 206, 2-(methoxymethyl)-5-methyl-N7-[cis-3-(trifluoromethoxy)cyclobutyl]pyrazolo[1,5-a]pyrimidine-3,7-dicarboxamide

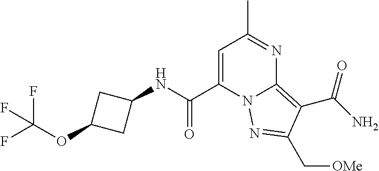 [SMILES: COCc1nn2c(C(=O)N[C@H]3C[C@@H](OC(F)(F)F)C3)cc(C)nc2c1C(N)=O]

2-(Methoxymethyl)-5-methyl-N7-[cis-3-(trifluoromethoxy)cyclobutyl]pyrazolo[1,5-a]pyrimidine-3,7-dicarboxamide (68 mg) was synthesised following a similar procedure to that described in General Method F.

¹H NMR (400 MHz, DMSO-d₆) δ 10.04 (d, J=7.3 Hz, 1H), 7.74 (s, 1H), 7.54 (s, 2H), 4.96 (s, 2H), 4.71 (p, J=7.2 Hz, 1H), 4.26-4.12 (m, 1H), 3.42 (s, 3H), 2.96-2.84 (m, 2H), 2.70 (s, 3H), 2.34-2.24 (m, 2H)

LCMS (Method 7): 2.44 min, 402.2 [M+H]⁺

### Synthesis of Compound 207, N7-(3,3-difluorocyclobutyl)-2-(hydroxymethyl)pyrazolo[1,5-a]pyrimidine-3,7-dicarboxamide

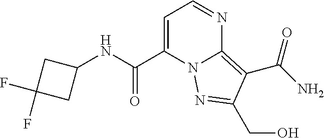 [SMILES: NC(=O)c1c(CO)nn2c(C(=O)NC3CC(F)(F)C3)ccnc12]

N7-(3,3-difluorocyclobutyl)-2-(hydroxymethyl)pyrazolo[1,5-a]pyrimidine-3,7-dicarboxamide (73 mg) was synthesised following a similar procedure to that described in General Method F.

¹H NMR (400 MHz, DMSO-d₆) δ 10.22 (d, J=5.3 Hz, 1H), 8.94 (d, J=4.4 Hz, 1H), 7.84 (s, 2H), 7.68 (d, J=4.4 Hz, 1H), 5.87 (t, J=6.6 Hz, 1H), 4.95 (d, J=6.6 Hz, 2H), 4.43-4.30 (m, 1H), 3.16-3.01 (m, 2H), 2.93-2.75 (m, 2H)

LCMS (Method 7): 1.85 min, 326.2 [M+H]⁺

### Synthesis of Compound 240, N7-(4,4-difluorocyclohexyl)-2-(methoxymethyl)-5-methyl-pyrazolo[1,5-a]pyrimidine-3,7-dicarboxamide

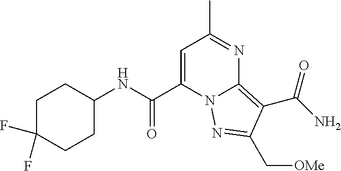 [SMILES: COCc1nn2c(C(=O)NC3CCC(F)(F)CC3)cc(C)nc2c1C(N)=O]

N7-(4,4-difluorocyclohexyl)-2-(methoxymethyl)-5-methyl-pyrazolo[1,5-a]pyrimidine-3,7-dicarboxamide (43 mg) was synthesised following a similar procedure to that described in General Method F.

¹H NMR (400 MHz, DMSO-d₆) δ 10.12 (d, J=7.5 Hz, 1H), 7.79-7.71 (m, 1H), 7.61 (s, 1H), 7.59-7.52 (m, 1H), 4.97 (s, 2H), 4.20-4.10 (m, 1H), 3.41 (s, 3H), 2.71 (s, 3H), 2.14-1.93 (m, 6H), 1.80-1.65 (m, 2H)

LCMS (Method 7): 2.32 min, 382.2 [M+H]⁺

### Synthesis of Compound 241, N7-(1,1-dioxo-3,4-dihydro-2H-thiochromen-4-yl)-2-(methoxymethyl)-5-methyl-pyrazolo[1,5-a]pyrimidine-3,7-dicarboxamide

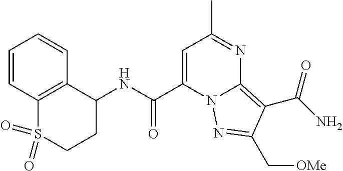 [SMILES: COCc1nn2c(C(=O)NC3CCS(=O)(=O)c4ccccc43)cc(C)nc2c1C(N)=O]

N7-(1,1-dioxo-3,4-dihydro-2H-thiochromen-4-yl)-2-(methoxymethyl)-5-methyl-pyrazolo[1,5-a]pyrimidine-3,7-dicarboxamide (51 mg) was synthesised following a similar procedure to that described in General Method F.

¹H NMR (400 MHz, DMSO-d₆) δ 10.28 (d, J=8.3 Hz, 1H), 7.85 (dd, J=7.8, 1.5 Hz, 1H), 7.76 (dt, J=7.8, 1.0 Hz, 1H), 7.74-7.70 (m, 1H), 7.68 (td, J=7.6, 1.5 Hz, 1H), 7.61 (td, J=7.7, 1.3 Hz, 1H), 7.58 (s, 1H), 7.56-7.50 (m, 1H), 5.65-5.55 (m, 1H), 4.92 (s, 2H), 3.88-3.77 (m, 1H), 3.75-3.64 (m, 1H), 3.33 (s, 3H), 2.71 (s, 3H), 2.69-2.62 (m, 1H), 2.61-2.52 (m, 1H)

LCMS (Method 7): 1.99 min, 444.2 [M+H]⁺

### Synthesis of Compound 242, 2-cyclopropyl-N7-(1,1-dioxo-3,4-dihydro-2H-thiochromen-4-yl)-5-methyl-pyrazolo[1,5-a]pyrimidine-3,7-dicarboxamide

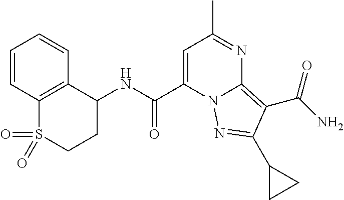 [SMILES: Cc1cc(C(=O)NC2CCS(=O)(=O)c3ccccc32)n2nc(C3CC3)c(C(N)=O)c2n1]

2-Cyclopropyl-N7-(1,1-dioxo-3,4-dihydro-2H-thiochromen-4-yl)-5-methyl-pyrazolo[1,5-a]pyrimidine-3,7-dicarboxamide (119 mg) was synthesised following a similar procedure to that described in General Method F.

¹H NMR (400 MHz, DMSO-d₆) δ 10.08 (d, J=8.0 Hz, 1H), 7.87 (dt, J=7.7, 1.0 Hz, 1H), 7.82 (d, J=3.0 Hz, 1H), 7.73-7.61 (m, 3H), 7.49 (s, 1H), 7.47 (d, J=2.9 Hz, 1H), 5.59-5.50 (m, 1H), 3.85-3.74 (m, 1H), 3.71-3.60 (m, 1H), 3.19-3.08 (m, 1H), 2.68 (s, 3H), 2.67-2.52 (m, 2H), 1.09-0.95 (m, 2H), 0.89-0.77 (m, 2H)

LCMS (Method 7): 2.22 min, 440.2 [M+H]⁺

### Synthesis of Compound 142, 2-(cyclopropylmethoxy)-N7-indan-2-yl-pyrazolo[1,5-a]pyrimidine-3,7-dicarboxamide (General Method G)

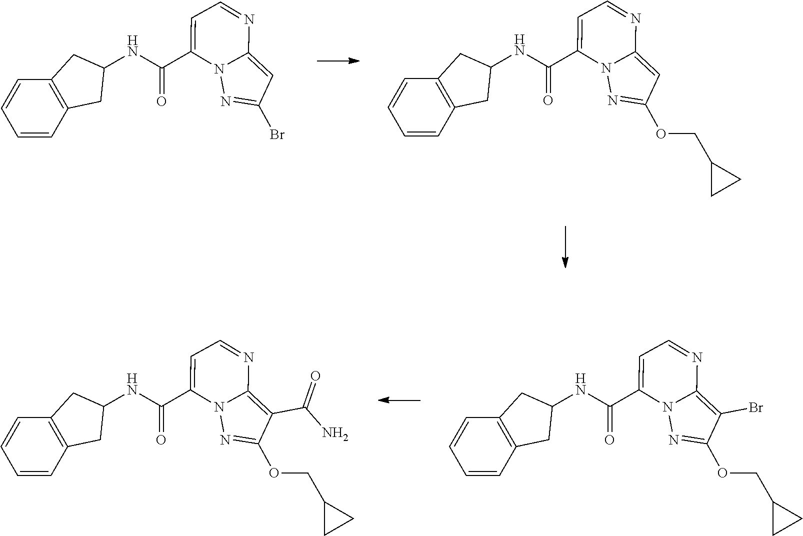 [SMILES: O=C(NC1Cc2ccccc2C1)c1ccnc2cc(Br)nn12 ; O=C(NC1Cc2ccccc2C1)c1ccnc2cc(OCC3CC3)nn12 ; NC(=O)c1c(OCC2CC2)nn2c(C(=O)NC3Cc4ccccc4C3)ccnc12 ; O=C(NC1Cc2ccccc2C1)c1ccnc2c(Br)c(OCC3CC3)nn12]

### Synthesis of 2-(cyclopropylmethoxy)-N-indan-2-yl-pyrazolo[1,5-a]pyrimidine-7-carboxamide

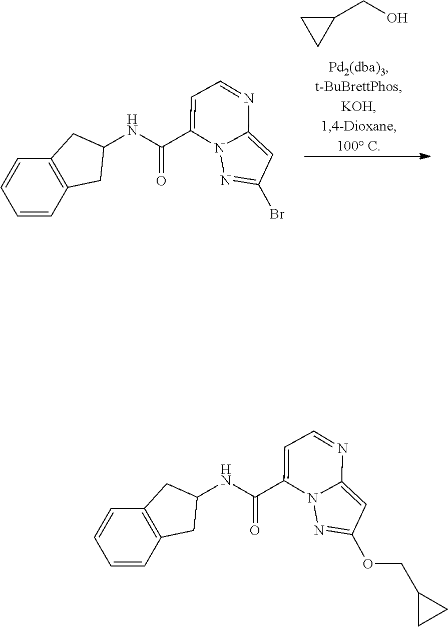 [SMILES: OCC1CC1 ; O=C(NC1Cc2ccccc2C1)c1ccnc2cc(Br)nn12 ; O=C(NC1Cc2ccccc2C1)c1ccnc2cc(OCC3CC3)nn12]

Cyclopropylmethanol (8.40 mL, 103.66 mmol) and KOH (94 mg, 1.68 mmol) were added to a solution of 2-bromo-N-indan-2-yl-pyrazolo[1,5-a]pyrimidine-7-carboxamide (300 mg, 0.84 mmol) in 1,4-dioxane (8.4 mL) and the solution was degassed by sparging with a stream of argon for 5 min. Pd₂(dba)₃ (39 mg, 0.04 mmol) and t-BuBrettPhos (41 mg, 0.08 mmol) were added, and the solution was degassed with argon for a further 5 min. The reaction was heated at 100° C. for 16 h then cooled to room temperature, filtered through a short pad of silica, and concentrated in vacuo. The residue was azeotroped with toluene (×3) and the crude product was purified by column chromatography (eluting with 0 to 50% EtOAc in heptane) to provide the title compound (191 mg).

¹H NMR (400 MHz, CDCl₃) δ 10.58 (d, J=7.6 Hz, 1H), 8.51 (d, J=4.4 Hz, 1H), 7.62 (d, J=4.4 Hz, 1H), 7.31-7.24 (m, 2H), 7.24-7.17 (m, 2H), 6.10 (s, 1H), 5.04-4.95 (m, 1H), 3.77 (d, J=7.2 Hz, 2H), 3.44 (dd, J=16.1, 6.7 Hz, 2H), 3.01 (dd, J=16.1, 3.8 Hz, 2H), 1.31-1.16 (m, 1H), 0.71-0.58 (m, 2H), 0.34 (dt, J=6.1, 4.7 Hz, 2H)

LCMS (Method 7): 3.25 min, 349.2 [M+H]⁺

### Synthesis of 3-bromo-2-(cyclopropylmethoxy)-N-indan-2-yl-pyrazolo[1,5-a]pyrimidine-7-carboxamide

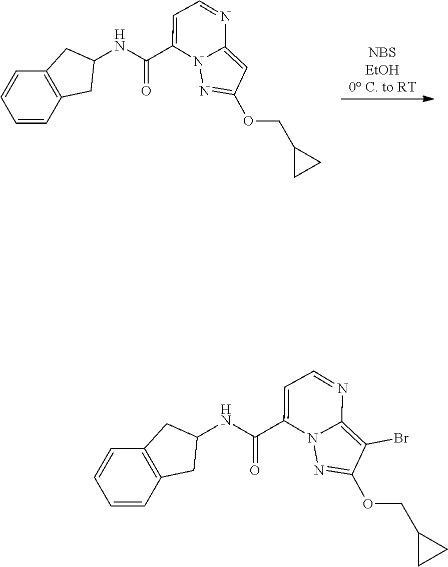 [SMILES: O=C(NC1Cc2ccccc2C1)c1ccnc2cc(OCC3CC3)nn12 ; O=C(NC1Cc2ccccc2C1)c1ccnc2c(Br)c(OCC3CC3)nn12]

NBS (117 mg, 0.65 mmol) was added to a suspension of 2-(cyclopropylmethoxy)-N-indan-2-yl-pyrazolo[1,5-a]pyrimidine-7-carboxamide (190 mg, 0.55 mmol) in EtOH (5.5 mL) at 0° C. and the reaction was stirred for 5 min. Additional EtOH (5.5 mL) was added, and the reaction was warmed to room temperature and stirred for a further 10 min. The reaction was quenched with sat. aq. Na₂S₂O₃ and diluted with EtOAc. The layers were separated, and the aqueous layer was extracted with EtOAc (×4). The combined organic layers were washed with brine, dried over Na₂SO₄, and concentrated in vacuo. The crude product was purified by column chromatography (eluting with 0 to 50% EtOAc in heptane) to provide the title compound (213 mg).

¹H NMR (400 MHz, CDCl₃) δ 10.32 (d, J=7.8 Hz, 1H), 8.61 (d, J=4.4 Hz, 1H), 7.68 (d, J=4.4 Hz, 1H), 7.31-7.27 (m, 2H), 7.25-7.19 (m, 2H), 5.05-4.97 (m, 1H), 3.79 (d, J=7.2 Hz, 2H), 3.44 (dd, J=16.2, 6.5 Hz, 2H), 2.99 (dd, J=16.2, 3.0 Hz, 2H), 1.32-1.17 (m, 1H), 0.70-0.59 (m, 2H), 0.43-0.31 (m, 2H)

LCMS (Method 7): 3.48 min, 427.0, 429.0 [M+H]⁺

### Synthesis of Compound 142, 2-(cyclopropylmethoxy)-N7-indan-2-yl-pyrazolo[1,5-a]pyrimidine-3,7-dicarboxamide

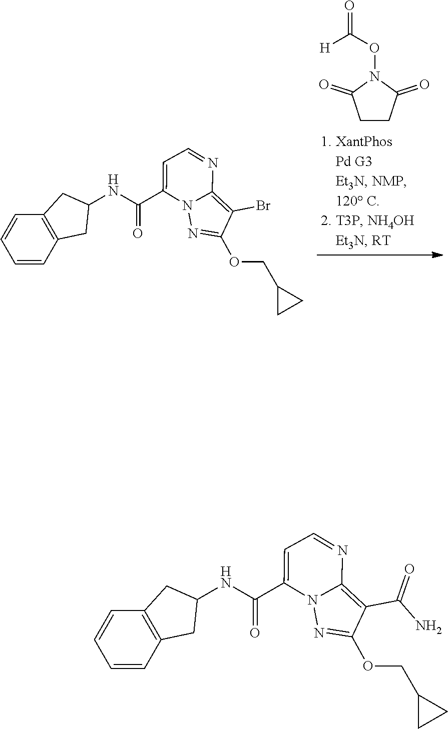 [SMILES: O=CON1C(=O)CCC1=O ; O=C(NC1Cc2ccccc2C1)c1ccnc2c(Br)c(OCC3CC3)nn12 ; NC(=O)c1c(OCC2CC2)nn2c(C(=O)NC3Cc4ccccc4C3)ccnc12]

Et₃N (200 μL, 1.40 mmol) was added to a vial containing a solution of 3-bromo-2-(cyclopropylmethoxy)-N-indan-2-yl-pyrazolo[1,5-a]pyrimidine-7-carboxamide (200 mg, 0.47 mmol) in NMP (4.7 mL). The vial was sealed, and the solution was degassed by sparging with a stream of argon for 5 min. XantPhos Pd G3 (45 mg, 0.05 mmol) was added, and the solution was degassed with argon for a further 5 min. (2,5-Dioxopyrrolidin-1-yl) formate (335 mg, 2.34 mmol) was added and the vial was sealed immediately. The reaction was heated at 120° C. in a microwave reactor for 2 h. Additional portions of XantPhos Pd G3 (45 mg, 0.05 mmol) and Et₃N (200 μL, 1.40 mmol) were added, and the mixture was purged with argon for 5 min. An additional portion of (2,5-dioxopyrrolidin-1-yl) formate (335 mg, 2.34 mmol) was added and the vial was sealed immediately. The reaction was heated at 120° C. in a microwave reactor for a further 2 h. The reaction mixture was cooled to room temperature, and Et₃N (200 μL, 1.40 mmol), NH₄OH (610 μL, 4.68 mmol) and 1-propanephosphonic anhydride (50 wt % in DMF) (560 μL, 0.94 mmol) were added. The reaction was stirred at room temperature for 16 h then quenched with water. The resulting precipitate was collected by filtration, washed with water, and dried. The crude product was purified by column chromatography (eluting with 0 to 10% MeOH in DCM) and preparative HPLC (basic method) to provide the title compound (70 mg).

¹H NMR (400 MHz, DMSO-d₆) δ 9.87 (d, J=7.3 Hz, 1H), 8.78 (d, J=4.5 Hz, 1H), 7.55 (d, J=4.5 Hz, 1H), 7.39 (br. s, 1H), 7.32-7.24 (m, 3H), 7.23-7.15 (m, 2H), 4.84-4.75 (m, 1H), 3.96 (d, J=7.2 Hz, 2H), 3.35 (dd, J=16.1, 6.8 Hz, 2H), 2.96 (dd, J=16.2, 3.8 Hz, 2H), 1.35-1.20 (m, 1H), 0.64-0.53 (m, 2H), 0.40-0.32 (m, 2H)

LCMS (Method 7): 2.61 min, 392.2 [M+H]⁺

### Synthesis of Compound 143, 7-(indan-2-ylcarbamoyl)-2-(methoxymethyl)pyrazolo[1,5-a]pyrimidine-3-carboxylic Acid (General Method H)

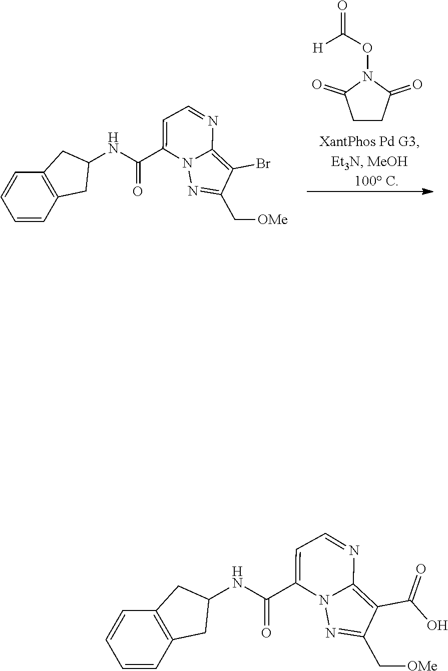 [SMILES: COCc1nn2c(C(=O)NC3Cc4ccccc4C3)ccnc2c1Br ; O=CON1C(=O)CCC1=O ; COCc1nn2c(C(=O)NC3Cc4ccccc4C3)ccnc2c1C(=O)O]

3-Bromo-N-indan-2-yl-2-(methoxymethyl)pyrazolo[1,5-a]pyrimidine-7-carboxamide (172 mg, 0.43 mmol), Et₃N (180 μL, 1.29 mmol), and MeOH (3.5 mL) were placed in a vial and the vial was sealed and purged with argon for 5 min. XantPhos Pd G3 (41 mg, 0.04 mmol) was added and the mixture was purged with argon for a further 5 min. (2,5-Dioxopyrrolidin-1-yl) formate (307 mg, 2.14 mmol) was added and the reaction was heated at 100° C. in a microwave reactor for 2 h. An additional portion of (2,5-dioxopyrrolidin-1-yl) formate (307 mg, 2.14 mmol) was added and the reaction was heated at 100° C. in a microwave reactor for a further 2 h. The reaction mixture was cooled to room temperature, diluted with water, and stirred for 2 h. The mixture was diluted with a 1:9 mixture of MeOH:DCM, filtered through a short pad of Dicalite®, and washed with a 1:9 mixture of MeOH:DCM and water. The layers were separated, and the combined organic layers were washed with brine, dried through a hydrophobic frit, and concentrated in vacuo. The crude product was purified by preparative HPLC (acidic method) to provide the title compound (23 mg).

¹H NMR (400 MHz, DMSO-d₆): δ 12.72 (s, 1H), 10.20 (d, J=7.2 Hz, 1H), 8.89 (d, J=4.3 Hz, 1H), 7.65 (d, J=4.3 Hz, 1H), 7.29 (dd, J=5.4, 3.2 Hz, 2H), 7.23-7.15 (m, 2H), 4.83 (s, 2H), 4.82-4.75 (m, 1H), 3.38 (dd, J=16.2, 7.3 Hz, 2H), 3.29 (s, 3H), 2.97 (dd, J=16.1, 4.6 Hz, 2H)

LCMS (Method 7): 2.41 min, 367.2 [M+H]⁺

### Synthesis of Compound 144, N7-(6-bromochroman-3-yl)-2-(methoxymethyl)pyrazolo[1,5-a]pyrimidine-3,7-dicarboxamide (General Method I)

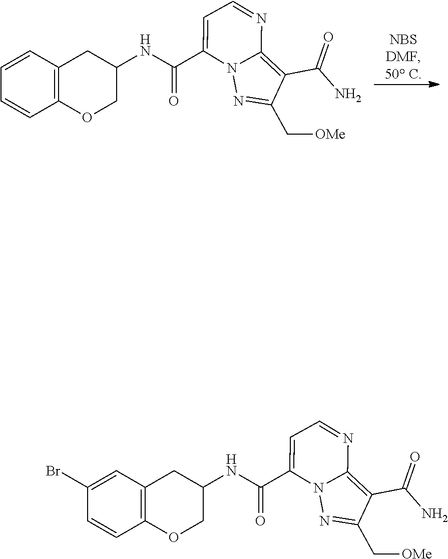 [SMILES: COCc1nn2c(C(=O)NC3COc4ccccc4C3)ccnc2c1C(N)=O ; COCc1nn2c(C(=O)NC3COc4ccc(Br)cc4C3)ccnc2c1C(N)=O]

NBS (19 mg, 0.14 mmol) was added to a solution of N7-chroman-3-yl-2-(methoxymethyl)pyrazolo[1,5-a]pyrimidine-3,7-dicarboxamide (50 mg, 0.13 mmol) in DMF (700 μL) and the reaction was heated at 50° C. for 4 h. The reaction mixture was cooled to room temperature and concentrated in vacuo. The crude product was purified by preparative HPLC (basic method) to provide the title compound (21 mg).

¹H NMR (400 MHz, DMSO-d₆) δ 10.30 (d, J=7.4 Hz, 1H), 8.92 (d, J=4.4 Hz, 1H), 7.76 (d, J=4.4 Hz, 1H), 7.67 (s, 1H), 7.58 (s, 1H), 7.36 (d, J=2.5 Hz, 1H), 7.29 (dd, J=8.7, 2.5 Hz, 1H), 6.83 (d, J=8.7 Hz, 1H), 4.87-4.74 (m, 2H), 4.65-4.57 (m, 1H), 4.26 (d, J=2.9 Hz, 2H), 3.29-3.24 (m, 4H), 2.89 (dd, J=17.0, 3.9 Hz, 1H) LCMS (Method 7): 2.48 min, 460.1, 462.1 [M+H]⁺

### Synthesis of Compound 145, 2-(methylamino)-N7-[cis-3-(trifluoromethoxy)cyclobutyl]pyrazolo[1,5-a]pyrimidine-3,7-dicarboxamide (General Method J)

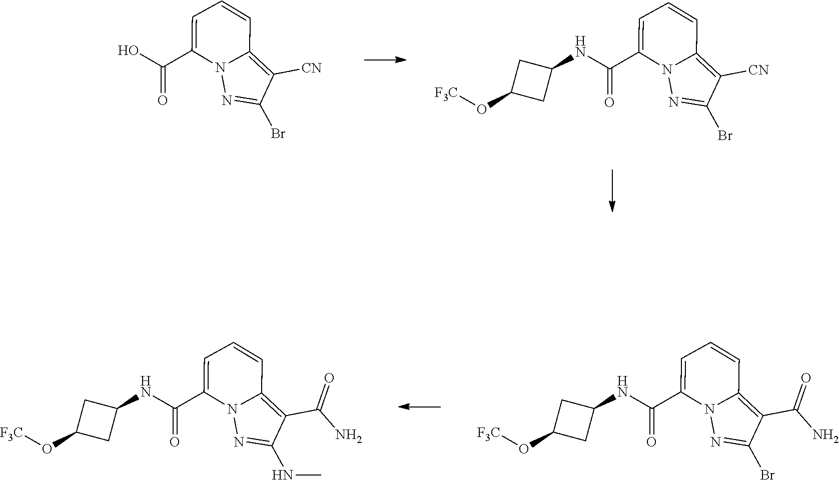 [SMILES: N#Cc1c(Br)nn2c(C(=O)O)cccc12 ; N#Cc1c(Br)nn2c(C(=O)N[C@H]3C[C@@H](OC(F)(F)F)C3)cccc12 ; CNc1nn2c(C(=O)N[C@H]3C[C@@H](OC(F)(F)F)C3)cccc2c1C(N)=O ; NC(=O)c1c(Br)nn2c(C(=O)N[C@H]3C[C@@H](OC(F)(F)F)C3)cccc12]

### Synthesis of 2-bromo-3-cyano-N-[cis-3-(trifluoromethoxy)cyclobutyl]pyrazolo[1,5-a]pyrimidine-7-carboxamide

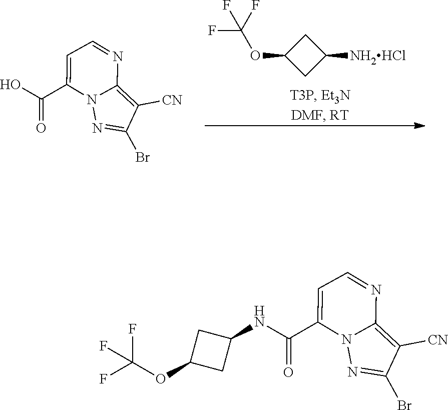

1-Propanephosphonic anhydride (50 wt % in DMF) (1.34 mL, 2.25 mmol) was added to a solution of 2-bromo-3-cyano-pyrazolo[1,5-a]pyrimidine-7-carboxylic acid (200 mg, 0.75 mmol), cis-3-(trifluoromethoxy)cyclobutanamine hydrochloride (215 mg, 1.12 mmol), and Et₃N (310 μL, 2.25 mmol) in DMF (2.5 mL) and the reaction was stirred at room temperature for 22 h. The reaction mixture was diluted with EtOAc and water, and the layers were separated. The aqueous layer was extracted with EtOAc (×3), and the combined organic layers were washed with brine, dried through a hydrophobic frit, and concentrated in vacuo. The crude product was purified by column chromatography (eluting with 0 to 80% EtOAc in heptane) to provide the title compound (225 mg).

¹H NMR (400 MHz, CDCl₃) δ 9.82 (d, J=6.8 Hz, 1H), 8.95 (d, J=4.4 Hz, 1H), 8.03 (d, J=4.4 Hz, 1H), 4.63-4.50 (m, 1H), 4.39-4.25 (m, 1H), 3.12-2.96 (m, 2H), 2.50-2.37 (m, 2H)

LCMS (Method 6): 2.08 min, 402.0, 404.0 [M−H]⁻

### Synthesis of 2-bromo-N7-[cis-3-(trifluoromethoxy)cyclobutyl]pyrazolo[1,5-a]pyrimidine-3,7-dicarboxamide

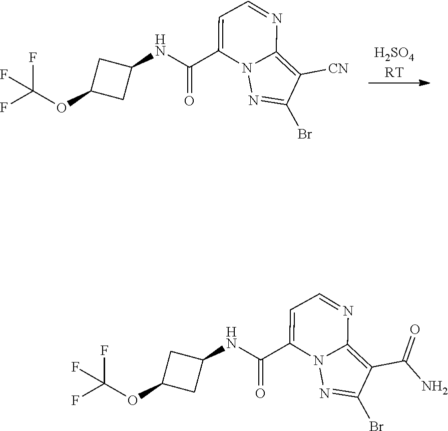 [SMILES: N#Cc1c(Br)nn2c(C(=O)N[C@H]3C[C@@H](OC(F)(F)F)C3)ccnc12 ; NC(=O)c1c(Br)nn2c(C(=O)N[C@H]3C[C@@H](OC(F)(F)F)C3)ccnc12]

2-Bromo-3-cyano-N-[cis-3-(trifluoromethoxy)cyclobutyl]pyrazolo[1,5-a]pyrimidine-7-carboxamide (118 mg, 0.29 mmol) was dissolved in conc. H₂SO₄ (580 μL, 10.74 mmol) and the reaction was stirred at room temperature for 2 h. The reaction mixture was cooled to 0° C. and ice-cold water was added. The mixture was basified with NH₃ (28-30 wt % in water) and the resulting precipitate was collected by filtration, washed with water and MeOH, and dried to provide the title compound (82 mg).

¹H NMR (400 MHz, DMSO-d₆) δ 9.60 (d, J=7.3 Hz, 1H), 8.93 (d, J=4.4 Hz, 1H), 7.68 (s, 2H), 7.56 (d, J=4.4 Hz, 1H), 4.70 (p, J=7.1 Hz, 1H), 4.26-4.12 (m, 1H), 2.96-2.80 (m, 2H), 2.38-2.26 (m, 2H)

LCMS (Method 6): 1.86 min, 422.0, 424.0 [M+H]⁺

### Synthesis of Compound 145, 2-(methylamino)-N7-[cis-3-(trifluoromethoxy)cyclobutyl]pyrazolo[1,5-a]pyrimidine-3,7-dicarboxamide

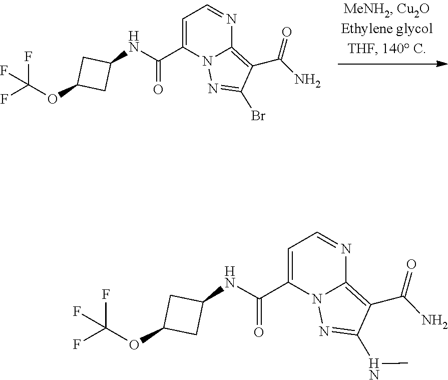 [SMILES: NC(=O)c1c(Br)nn2c(C(=O)N[C@H]3C[C@@H](OC(F)(F)F)C3)ccnc12 ; CNc1nn2c(C(=O)N[C@H]3C[C@@H](OC(F)(F)F)C3)ccnc2c1C(N)=O]

2-Bromo-N7-[cis-3-(trifluoromethoxy)cyclobutyl]pyrazolo[1,5-a]pyrimidine-3,7-dicarboxamide (110 mg, 0.26 mmol), Cu₂O (4 mg, 0.03 mmol), and MeNH₂ (2 M in THF) (3.9 mL, 7.82 mmol) were suspended in ethylene glycol (1 mL) and the reaction was heated at 140° C. in a microwave reactor for 1 h. The reaction mixture was cooled to room temperature, diluted with MeOH, and filtered through a short pad of Dicalite®. The filtrate was concentrated in vacuo, and the crude product was purified by column chromatography (eluting with 0 to 5% MeOH in DCM) to provide the title compound (22 mg).

¹H NMR (400 MHz, CDCl₃) δ 10.88 (d, J=7.0 Hz, 1H), 8.52 (d, J=4.6 Hz, 1H), 7.68 (s, 1H), 7.63 (d, J=4.6 Hz, 1H), 6.93 (s, 1H), 5.50 (s, 1H), 4.54 (p, J=7.2 Hz, 1H), 4.37-4.22 (m, 1H), 3.12 (d, J=2.9 Hz, 3H), 3.10-3.00 (m, 2H), 2.38-2.26 (m, 2H)

LCMS (Method 7): 2.64 min, 373.2 [M+H]⁺

### Synthesis of Compound 148, 7-(indan-2-ylcarbamothioyl)-2-(methoxymethyl)pyrazolo[1,5-a]pyrimidine-3-carboxamide (General Method K)

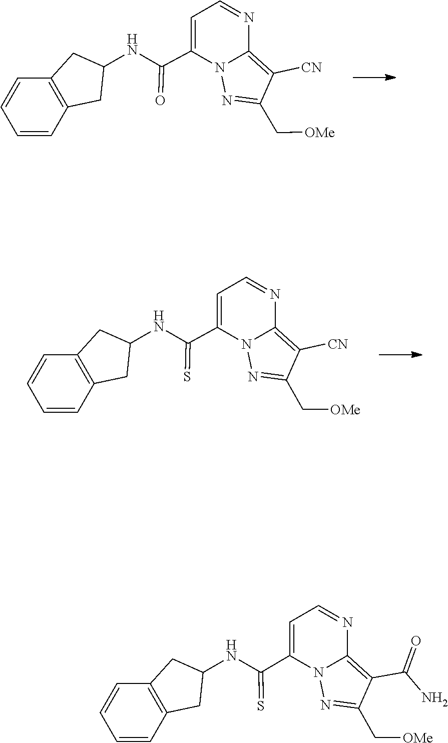 [SMILES: COCc1nn2c(C(=O)NC3Cc4ccccc4C3)ccnc2c1C#N ; COCc1nn2c(C(=S)NC3Cc4ccccc4C3)ccnc2c1C#N ; COCc1nn2c(C(=S)NC3Cc4ccccc4C3)ccnc2c1C(N)=O]

### Synthesis of 3-cyano-N-indan-2-yl-2-(methoxymethyl)pyrazolo[1,5-a]pyrimidine-7-carbothioamide

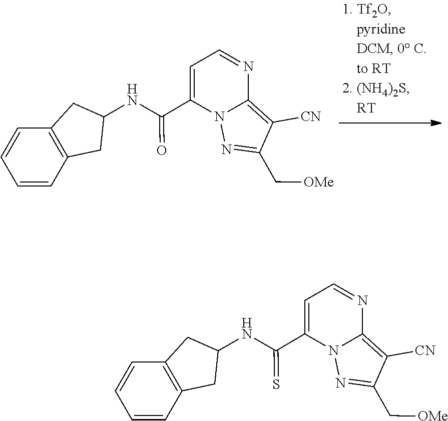 [SMILES: COCc1nn2c(C(=O)NC3Cc4ccccc4C3)ccnc2c1C#N ; COCc1nn2c(C(=S)NC3Cc4ccccc4C3)ccnc2c1C#N]

Tf₂O (340 μL, 2.03 mmol) was added to a solution of 3-cyano-N-indan-2-yl-2-(methoxymethyl)pyrazolo[1,5-a]pyrimidine-7-carboxamide (300 mg, 0.85 mmol) and pyridine (410 μL, 5.08 mmol) in DCM (4 mL) at 0° C. The reaction was stirred at 0° C. for 2 h then warmed to room temperature and stirred for 3.5 days. (NH₄)₂S (20 wt % in water) (870 μL, 2.54 mmol) was added, and the reaction was stirred for 1 h. The reaction mixture was diluted with DCM and water, and the layers were separated. The aqueous layer was extracted with DCM, and the combined organic layers were washed with water and brine, dried over Na₂SO₄, and concentrated in vacuo to provide the title compound (329 mg).

¹H NMR (400 MHz, CDCl₃) δ 12.45 (s, 1H), 8.85 (d, J=4.6 Hz, 1H), 8.53 (d, J=4.6 Hz, 1H), 7.34-7.29 (m, 2H), 7.27-7.22 (m, 2H), 5.49-5.37 (m, 1H), 4.61 (s, 2H), 3.59 (dd, J=16.3, 6.9 Hz, 2H), 3.42 (s, 3H), 3.16 (dd, J=16.3, 4.0 Hz, 2H)

LCMS (Method 6): 2.22 min, 364.2 [M+H]⁺

### Synthesis of Compound 148, 7-(indan-2-ylcarbamothioyl)-2-(methoxymethyl)pyrazolo[1,5-a]pyrimidine-3-carboxamide

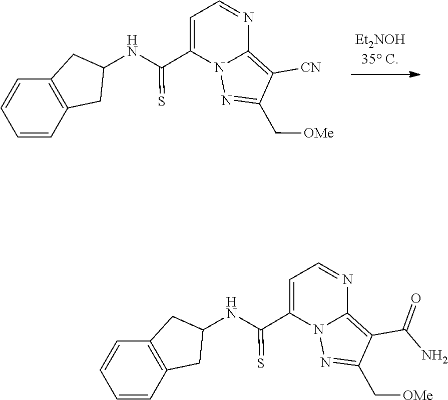 [SMILES: COCc1nn2c(C(=S)NC3Cc4ccccc4C3)ccnc2c1C#N ; COCc1nn2c(C(=S)NC3Cc4ccccc4C3)ccnc2c1C(N)=O]

3-cyano-N-indan-2-yl-2-(methoxymethyl)pyrazolo[1,5-a]pyrimidine-7-carbothioamide (300 mg, 0.70 mmol) was dissolved in N,N-diethylhydroxylamine (6 mL, 58.36 mmol) and the reaction was stirred at 35° C. for 3 days. The reaction mixture was cooled to room temperature and diluted with water and EtOAc. The layers were separated, and the aqueous layer was extracted with EtOAc. The combined organic layers were washed with water (×3) and brine, dried over Na₂SO₄, and concentrated in vacuo. The crude product was triturated with MeCN and dried to provide the title compound (82 mg).

¹H NMR (400 MHz, CDCl₃) δ 13.12 (s, 1H), 8.75 (d, J=4.6 Hz, 1H), 8.50 (d, J=4.6 Hz, 1H), 8.04 (s, 1H), 7.30 (dd, J=5.4, 3.3 Hz, 2H), 7.24-7.21 (m, 2H), 5.59 (s, 1H), 5.45-5.34 (m, 1H), 5.03 (s, 2H), 3.62 (dd, J=16.3, 7.2 Hz, 2H), 3.41 (s, 3H), 3.21 (dd, J=16.3, 4.4 Hz, 2H)

LCMS (Method 7): 2.77 min, 382.2 [M+H]⁺

### Synthesis of Compound 149, 3-carbamothioyl-N-indan-2-yl-2-(methoxymethyl)pyrazolo[1,5-a]pyrimidine-7-carboxamide (General Method L)

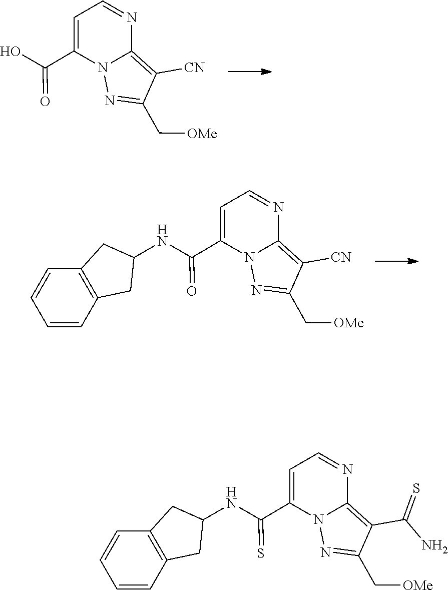 [SMILES: COCc1nn2c(C(=O)O)ccnc2c1C#N ; COCc1nn2c(C(=O)NC3Cc4ccccc4C3)ccnc2c1C#N ; COCc1nn2c(C(=S)NC3Cc4ccccc4C3)ccnc2c1C(N)=S]

### Synthesis of 3-cyano-N-indan-2-yl-2-(methoxymethyl)pyrazolo[1,5-a]pyrimidine-7-carboxamide

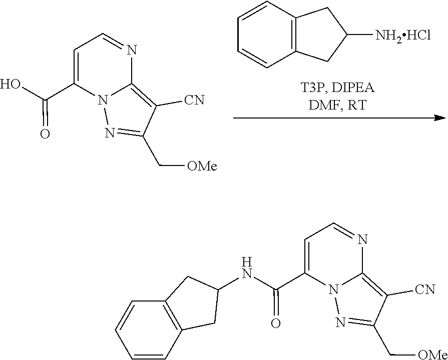

1-Propanephosphonic anhydride (50 wt % in EtOAc) (2.37 mL, 3.99 mmol) was added to a solution of 3-cyano-2-(methoxymethyl)pyrazolo[1,5-a]pyrimidine-7-carboxylic acid (630 mg, 2.66 mmol), indan-2-amine hydrochloride (541 mg, 3.19 mmol), and DIPEA (930 μL, 5.32 mmol) in DMF (5 mL), and the reaction was stirred at room temperature overnight. The reaction mixture was diluted with water, and the resulting precipitate was collected by filtration, washed with water, and dried to provide the title compound (890 mg).

¹H NMR (400 MHz, CDCl₃) δ 10.28 (d, J=7.3 Hz, 1H), 8.91 (d, J=4.3 Hz, 1H), 8.04 (d, J=4.3 Hz, 1H), 7.33-7.20 (m, 4H), 5.08-4.95 (m, 1H), 4.69 (s, 2H), 3.55-3.45 (m, 5H), 3.05 (dd, J=16.0, 5.0 Hz, 2H)

LCMS (Method 6): 2.04 min, 348.2 [M+H]⁺

### Synthesis of Compound 149, 3-carbamothioyl-N-indan-2-yl-2-(methoxymethyl)pyrazolo[1,5-a]pyrimidine-7-carboxamide

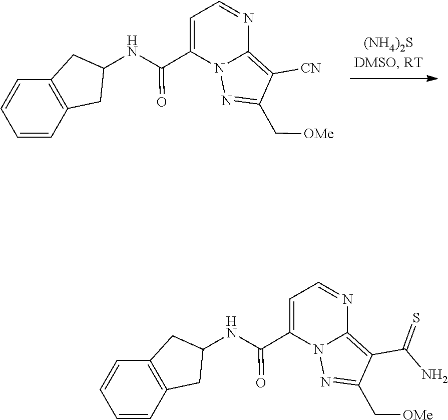 [SMILES: COCc1nn2c(C(=O)NC3Cc4ccccc4C3)ccnc2c1C#N ; COCc1nn2c(C(=O)NC3Cc4ccccc4C3)ccnc2c1C(N)=S]

(NH₄)₂S (20 wt % in water) (490 μL, 1.44 mmol) was added to a solution of 3-cyano-N-indan-2-yl-2-(methoxymethyl)pyrazolo[1,5-a]pyrimidine-7-carboxamide (100 mg, 0.29 mmol) in DMSO (3 mL) and the reaction was stirred at room temperature for 24 h. An additional portion of (NH₄)₂S (20 wt % in water) (490 μL, 1.44 mmol) was added, and the reaction was stirred for a further 48 h. A third portion of (NH₄)₂S (20 wt % in water) (490 UL, 1.44 mmol) was added, followed by additional DMSO (1 mL), and the reaction was stirred for a further 5 h. The resulting precipitate was collected by filtration, washed with water and EtOAc, and dried to provide the title compound (27 mg).

¹H NMR (400 MHz, DMSO-d₆) δ 10.22 (d, J=7.0 Hz, 1H), 9.71 (s, 1H), 9.57 (s, 1H), 8.93 (d, J=4.4 Hz, 1H), 7.72 (d, J=4.4 Hz, 1H), 7.33-7.24 (m, 2H), 7.22-7.14 (m, 2H), 5.13 (s, 2H), 4.86-4.75 (m, 1H), 3.40 (dd, J=16.2, 7.3 Hz, 2H), 3.33 (s, 3H), 2.97 (dd, J=16.2, 4.5 Hz, 2H)

LCMS (Method 6): 2.06 min, 382.2 [M+H]⁺

### Synthesis of Compound 150, 2-cyclopropyl-N8-(4,4-difluorocyclohexyl) imidazo[1,2-b]pyridazine-3,8-dicarboxamide (General Method M)

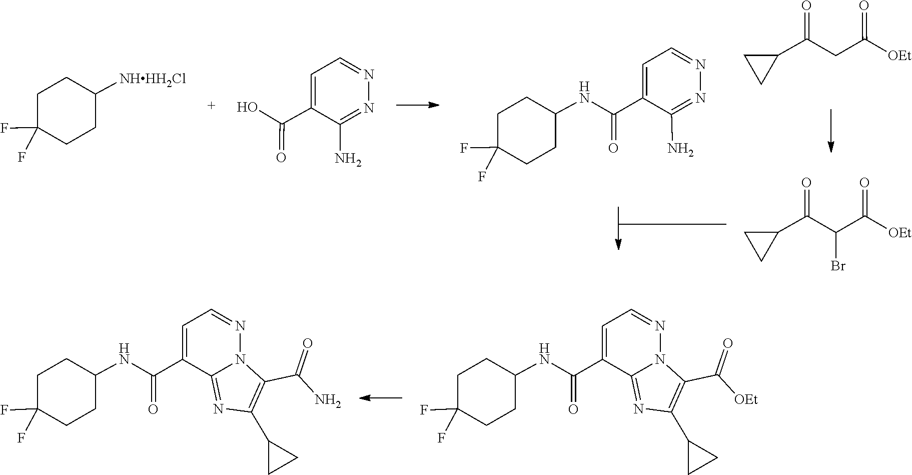

### Synthesis of 3-amino-N-(4,4-difluorocyclohexyl)pyridazine-4-carboxamide

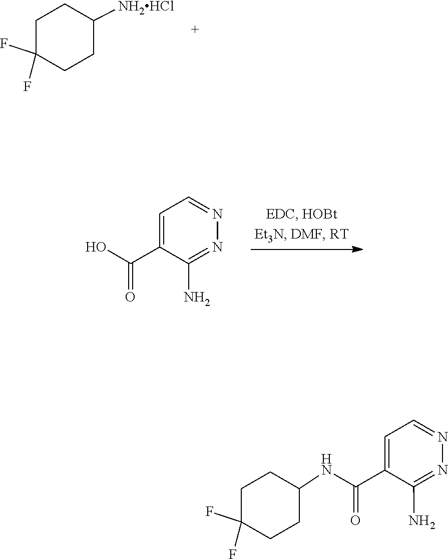

EDC (4.13 g, 21.57 mmol) was added to a solution of 3-aminopyridazine-4-carboxylic acid (1 g, 7.19 mmol), 4,4-difluorocyclohexanamine hydrochloride (1.48 g, 8.63 mmol), Et₃N (5.01 mL, 35.94 mmol), and HOBt (3.3 g, 21.57 mmol) in DMF (9.5 mL), and the reaction was stirred at room temperature for 20 h. The reaction mixture was concentrated in vacuo, and the crude product was purified by column chromatography (eluting with 0 to 100% EtOAc in heptane) and SCX chromatography (eluting with 2 M NH₃ in MeOH) to provide the title compound (1.19 g).

¹H NMR (400 MHz, DMSO-d₆) δ 8.64 (d, J=7.6 Hz, 1H), 8.60 (d, J=4.9 Hz, 1H), 7.57 (d, J=4.9 Hz, 1H), 7.04 (s, 2H), 4.02-3.90 (m, 1H), 2.12-1.82 (m, 6H), 1.67-1.55 (m, 2H)

LCMS (Method 6): 1.13 min, 257.2 [M+H]⁺

### Synthesis of ethyl 2-bromo-3-cyclopropyl-3-oxo-propanoate

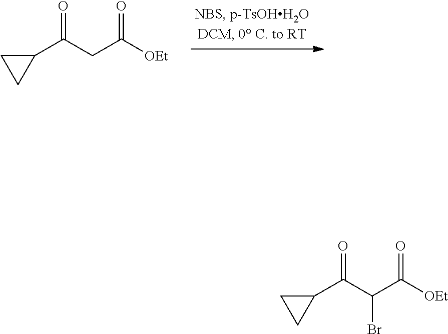 [SMILES: CCOC(=O)CC(=O)C1CC1 ; CCOC(=O)C(Br)C(=O)C1CC1]

NBS (2.39 g, 13.45 mmol) and p-TsOH. H₂O were added to a solution of ethyl 3-cyclopropyl-3-oxo-propanoate (1.89 mL, 12.81 mmol) in DCM (50 mL) at 0° C., and the reaction was stirred at 0° C. for 10 min then warmed to room temperature and stirred for 18 h. The reaction mixture was diluted with DCM and washed with brine (×2), and the organic layer was dried over Na₂SO₄ and concentrated in vacuo. The crude product was purified by column chromatography (eluting with 0 to 10% EtOAc in heptane) to provide the title compound (2.84 g).

¹H NMR (400 MHz, CDCl₃) δ 4.90 (s, 1H), 4.30 (q, J=7.1 Hz, 2H), 2.34-2.27 (m, 1H), 1.31 (t, J=7.1 Hz, 3H), 1.20-1.16 (m, 2H), 1.09-1.04 (m, 2H)

### Synthesis of ethyl 2-cyclopropyl-8-[(4,4-difluorocyclohexyl) carbamoyl]imidazo[1,2-b]pyridazine-3-carboxylate

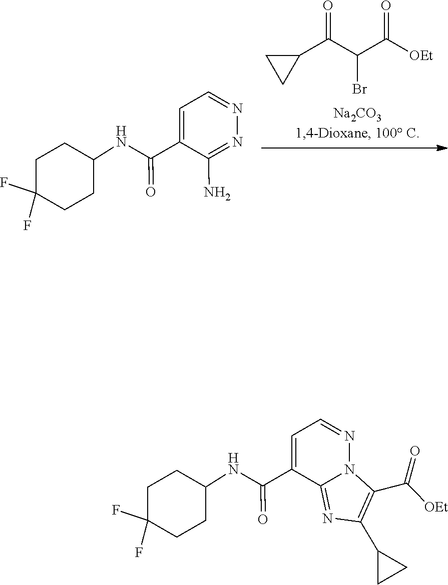 [SMILES: CCOC(=O)C(Br)C(=O)C1CC1 ; Nc1nnccc1C(=O)NC1CCC(F)(F)CC1 ; CCOC(=O)c1c(C2CC2)nc2c(C(=O)NC3CCC(F)(F)CC3)ccnn12]

3-Amino-N-(4,4-difluorocyclohexyl)pyridazine-4-carboxamide (600 mg, 2.34 mmol), ethyl 2-bromo-3-cyclopropyl-3-oxo-propanoate (826 mg, 3.51 mmol), and Na₂CO₃ (744 mg, 7.02 mmol) were dissolved in 1,4-dioxane (9.5 mL), and the reaction was heated at 100° C. for 18 h. The reaction mixture was cooled to room temperature and concentrated in vacuo. The crude product was purified by column chromatography (eluting with 0 to 40% EtOAc in heptane) to provide the title compound (117 mg).

¹H NMR (400 MHz, CDCl₃) δ 9.97 (d, J=7.6 Hz, 1H), 8.67 (d, J=4.7 Hz, 1H), 7.97 (d, J=4.7 Hz, 1H), 4.53 (q, J=7.1 Hz, 2H), 4.24-4.18 (m, 1H), 2.95-2.87 (m, 1H), 2.15-1.96 (m, 6H), 1.84-1.76 (m, 2H), 1.47 (t, J=7.1 Hz, 3H), 1.24-1.17 (m, 2H), 1.17-1.11 (m, 2H)

LCMS (Method 6): 2.21 min, 393.2 [M+H]⁺

### Synthesis of Compound 150, 2-cyclopropyl-N8-(4,4-difluorocyclohexyl) imidazo[1,2-b]pyridazine-3,8-dicarboxamide

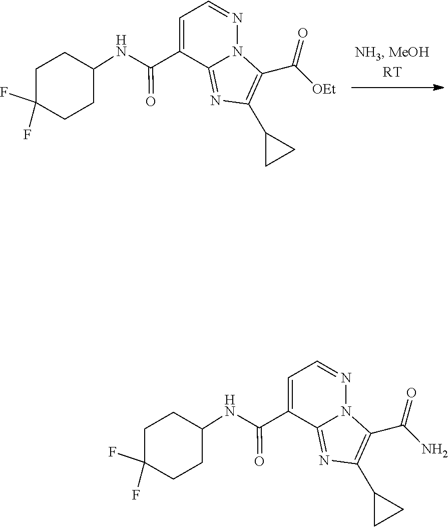 [SMILES: CCOC(=O)c1c(C2CC2)nc2c(C(=O)NC3CCC(F)(F)CC3)ccnn12 ; NC(=O)c1c(C2CC2)nc2c(C(=O)NC3CCC(F)(F)CC3)ccnn12]

Ethyl 2-cyclopropyl-8-[(4,4-difluorocyclohexyl) carbamoyl]imidazo[1,2-b]pyridazine-3-carboxylate (117 mg, 0.27 mmol) was dissolved in NH₃ (7 M in MeOH) (3.83 mL, 26.83 mmol) and the reaction was stirred at room temperature for 64 h. The reaction mixture was concentrated in vacuo, and the crude product was purified by column chromatography (eluting with 0 to 4% MeOH in DCM) and preparative HPLC (basic method) to provide the title compound (39 mg).

¹H NMR (400 MHz, DMSO-d₆) δ 9.76 (d, J=7.5 Hz, 1H), 8.83 (d, J=4.8 Hz, 1H), 8.23 (s, 1H), 8.00 (s, 1H), 7.87 (d, J=4.8 Hz, 1H), 4.17-4.08 (m, 1H), 3.26-3.15 (m, 1H), 2.10-1.94 (m, 6H), 1.74-1.64 (m, 2H), 1.19-1.11 (m, 2H), 1.07-0.99 (m, 2H)

LCMS (Method 7): 2.51 min, 364.2 [M+H]⁺

### Synthesis of Compound 151, 2-cyclopropyl-N8-[cis-3-(trifluoromethoxy)cyclobutyl]imidazo[1,2-b]pyridazine-3,8-dicarboxamide

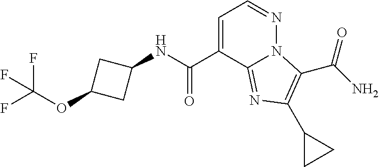 [SMILES: NC(=O)c1c(C2CC2)nc2c(C(=O)N[C@H]3C[C@@H](OC(F)(F)F)C3)ccnn12]

2-Cyclopropyl-N8-[cis-3-(trifluoromethoxy)cyclobutyl]imidazo[1,2-b]pyridazine-3,8-dicarboxamide (24 mg) was synthesised following a similar procedure to that described in General Method M.

¹H NMR (400 MHz, DMSO-d₆) δ 9.81 (d, J=7.5 Hz, 1H), 8.82 (d, J=4.8 Hz, 1H), 8.22 (s, 1H), 7.99 (s, 1H), 7.82 (d, J=4.8 Hz, 1H), 4.72 (p, J=7.1 Hz, 1H), 4.24-4.13 (m, 1H), 3.26-3.17 (m, 1H), 2.96-2.86 (m, 2H), 2.31-2.19 (m, 2H), 1.19-1.08 (m, 4H)

LCMS (Method 4): 2.41 min, 384.0 [M+H]⁺

### Synthesis of Compound 208, 2-cyclopropyl-N8-(5,6-dihydro-4H-cyclopenta[c]thiophen-5-yl) imidazo[1,2-b]pyridazine-3,8-dicarboxamide

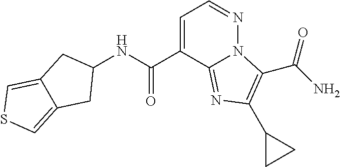 [SMILES: NC(=O)c1c(C2CC2)nc2c(C(=O)NC3Cc4cscc4C3)ccnn12]

2-Cyclopropyl-N8-(5,6-dihydro-4H-cyclopenta[c]thiophen-5-yl) imidazo[1,2-b]pyridazine-3,8-dicarboxamide (25 mg) was synthesised following a similar procedure to that described in General Method M.

¹H NMR (400 MHz, DMSO-d₆) δ 10.06 (d, J=7.5 Hz, 1H), 8.82 (d, J=4.8 Hz, 1H), 8.20 (s, 1H), 7.95 (s, 1H), 7.87 (d, J=4.8 Hz, 1H), 7.14 (s, 2H), 5.02-4.93 (m, 1H), 3.20-3.09 (m, 3H), 2.68 (dd, J=15.7, 4.6 Hz, 2H), 1.09-1.00 (m, 2H), 0.91-0.82 (m, 2H)

LCMS (Method 7): 2.54 min, 368.2 [M+H]⁺

### Synthesis of Compound 209, 2-cyclopropyl-N8-(3,3-difluoroindan-1-yl) imidazo[1,2-b]pyridazine-3,8-dicarboxamide

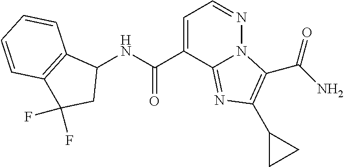 [SMILES: NC(=O)c1c(C2CC2)nc2c(C(=O)NC3CC(F)(F)c4ccccc43)ccnn12]

2-Cyclopropyl-N8-(3,3-difluoroindan-1-yl) imidazo[1,2-b]pyridazine-3,8-dicarboxamide (31 mg) was synthesised following a similar procedure to that described in General Method M.

¹H NMR (400 MHz, DMSO-d₆) δ 10.15 (d, J=7.3 Hz, 1H), 8.85 (d, J=4.8 Hz, 1H), 8.21 (s, 1H), 7.97 (s, 1H), 7.91 (d, J=4.8 Hz, 1H), 7.73-7.54 (m, 4H), 5.71-5.62 (m, 1H), 3.28-3.07 (m, 2H), 2.67-2.54 (m, 1H), 1.04-0.95 (m, 2H), 0.86-0.79 (m, 2H)

LCMS (Method 7): 2.65 min, 398.2 [M+H]⁺

### Synthesis of Compound 210, N8-(7-bicyclo[4.2.0]octa-1,3,5-trienyl)-2-cyclopropyl-imidazo[1,2-b]pyridazine-3,8-dicarboxamide

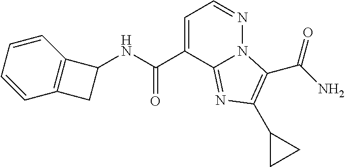 [SMILES: NC(=O)c1c(C2CC2)nc2c(C(=O)NC3Cc4ccccc43)ccnn12]

N8-(7-bicyclo[4.2.0]octa-1,3,5-trienyl)-2-cyclopropyl-imidazo[1,2-b]pyridazine-3,8-dicarboxamide (10 mg) was synthesised following a similar procedure to that described in General Method M.

¹H NMR (400 MHz, DMSO-d₆) δ 10.28 (d, J=6.5 Hz, 1H), 8.85 (d, J=4.8 Hz, 1H), 8.22 (s, 1H), 7.97 (s, 1H), 7.90 (d, J=4.8 Hz, 1H), 7.41-7.21 (m, 4H), 5.52-5.46 (m, 1H), 3.77 (dd, J=14.3, 4.9 Hz, 1H), 3.19-3.08 (m, 2H), 1.09-0.97 (m, 2H), 0.96-0.81 (m, 2H)

LCMS (Method 7): 2.63 min, 348.2 [M+H]⁺

### Synthesis of Compound 211, 2-cyclopropyl-N8-[(4S)-7-fluorochroman-4-yl]imidazo[1,2-b]pyridazine-3,8-dicarboxamide

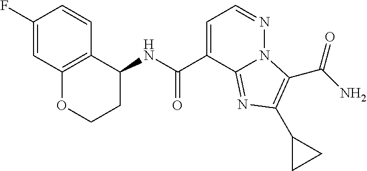 [SMILES: NC(=O)c1c(C2CC2)nc2c(C(=O)N[C@H]3CCOc4cc(F)ccc43)ccnn12]

2-Cyclopropyl-N8-[(4S)-7-fluorochroman-4-yl]imidazo[1,2-b]pyridazine-3,8-dicarboxamide (35 mg) was synthesised following a similar procedure to that described in General Method M.

¹H NMR (400 MHz, DMSO-d₆) δ 10.06 (d, J=7.4 Hz, 1H), 8.85 (d, J=4.8 Hz, 1H), 8.21 (s, 1H), 7.98 (s, 1H), 7.90 (d, J=4.8 Hz, 1H), 7.39 (dd, J=8.5, 6.7 Hz, 1H), 6.84-6.69 (m, 2H), 5.27-5.18 (m, 1H), 4.45-4.34 (m, 1H), 4.24-4.14 (m, 1H), 3.16-3.08 (m, 1H), 2.32-2.24 (m, 1H), 2.13-2.05 (m, 1H), 1.09-0.95 (m, 2H), 0.86-0.70 (m, 2H)

LCMS (Method 7): 2.68 min, 396.2 [M+H]⁺

### Synthesis of Compound 156, 7-cyclopropyl-N4-(4,4-difluorocyclohexyl)pyrazolo[5,1-c][1,2,4]triazine-4,8-dicarboxamide (General Method N)

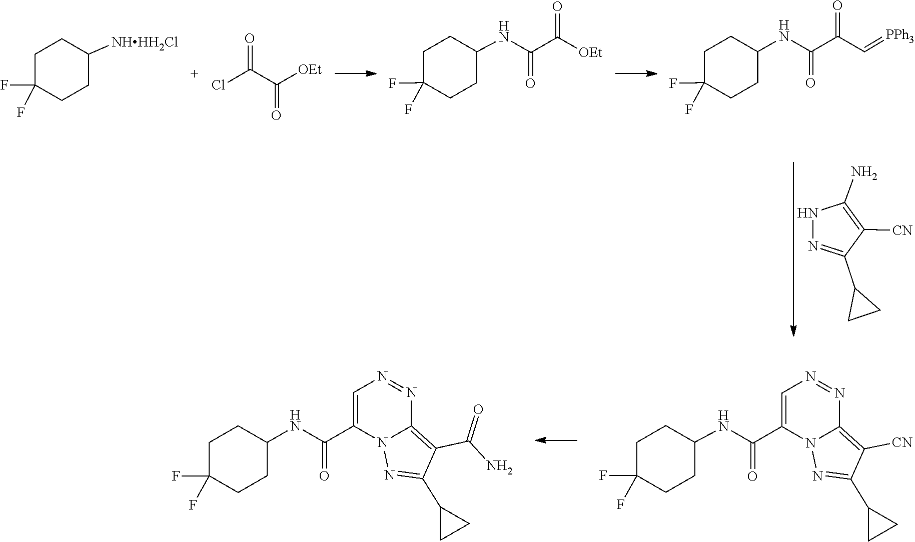

### Synthesis of ethyl 2-[(4,4-difluorocyclohexyl)amino]-2-oxo-acetate

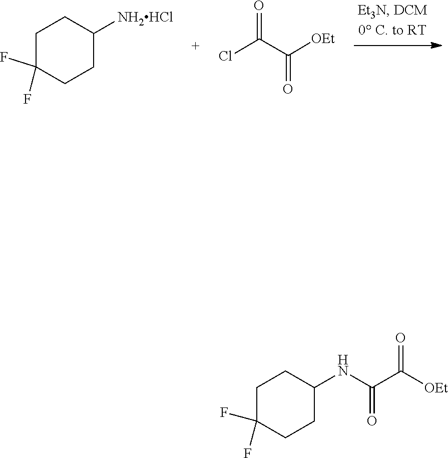

Et₃N (6.6 mL, 47.6 mmol) and ethyl 2-chloro-2-oxo-acetate (2.7 mL, 23.8 mmol) were added sequentially to a suspension of 4,4-difluorocyclohexanamine hydrochloride (3.4 g, 19.80 mmol) in DCM (30 mL) at 0° C. and the reaction was warmed to room temperature and stirred for 20 h. The reaction was quenched with sat. aq. NH₄Cl and diluted with DCM. The layers were separated, and the aqueous layer was extracted with DCM. The combined organic layers were dried through a hydrophobic frit and concentrated in vacuo. The crude product was purified by column chromatography (eluting with 0 to 100% EtOAc in heptane) to provide the title compound (3.65 g).

¹H NMR (400 MHz, CDCl₃) δ 7.02 (s, 1H), 4.35 (q, J=7.1 Hz, 2H), 3.99-3.84 (m, 1H), 2.20-1.77 (m, 6H), 1.70-1.59 (m, 2H), 1.39 (t, J=7.1 Hz, 3H)

LCMS (Method 6): 0.98 min, 236.2 [M+H]⁺

### Synthesis of N-(4,4-difluorocyclohexyl)-2-oxo-3-(triphenyl-λ5-phosphanylidene)propenamide

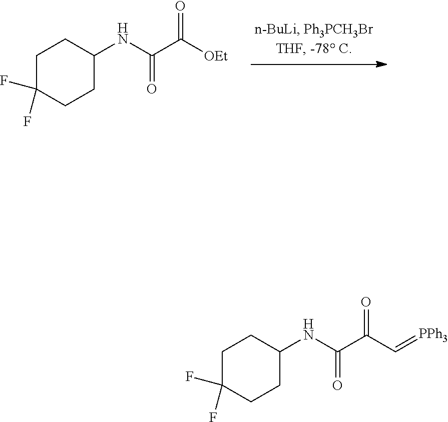

n-BuLi (2.5 M in hexanes) (6.8 mL, 16.90 mmol) was added dropwise to a suspension of methyl(triphenyl)phosphonium bromide (6 g, 16.90 mmol) in THF (15 mL) at −78° C. and the mixture was stirred at −78° C. for 30 min. A solution of ethyl 2-[(4,4-difluorocyclohexyl)amino]-2-oxo-acetate (1.6 g, 6.80 mmol) in THF (15 mL) was added dropwise, and the reaction was stirred at −78° C. for a further 45 min. The reaction was quenched with water and diluted with EtOAc. The layers were separated, and the aqueous layer was extracted with EtOAc. The combined organic layers were dried over Na₂SO₄ and concentrated in vacuo. The crude product was purified by column chromatography (eluting with 0 to 100% EtOAc in heptane) to provide the title compound (907 mg).

¹H NMR (400 MHz, DMSO-d₆) δ 8.05 (d, J=8.3 Hz, 1H), 7.71-7.56 (m, 15H), 4.74 (d, J=25.0 Hz, 1H), 3.77-3.65 (m, 1H), 2.01-1.94 (m, 2H), 1.95-1.81 (m, 2H), 1.79-1.72 (m, 2H), 1.63-1.50 (m, 2H)

LCMS (Method 6): 1.20 min, 466.2 [M+H]⁺

### Synthesis of 8-cyano-7-cyclopropyl-N-(4,4-difluorocyclohexyl)pyrazolo[5,1-c][1,2,4]triazine-4-carboxamide

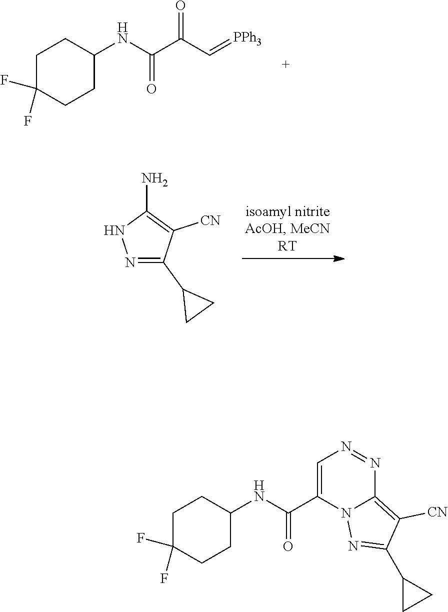

Isoamyl nitrite (350 μL, 2.61 mmol) was added to a suspension of N-(4,4-difluorocyclohexyl)-2-oxo-3-(triphenyl-λ5-phosphanylidene) propanamide (1.3 g, 2.70 mmol) and 5-amino-3-cyclopropyl-1H-pyrazole-4-carbonitrile (200 mg, 1.40 mmol) in MeCN (10 mL) and AcOH (230 μL, 4.1 mmol), and the reaction was stirred at room temperature for 2 h. The reaction mixture was concentrated in vacuo, and the crude product was purified by column chromatography (eluting with 0 to 100% EtOAc in heptane) to provide the title compound (80 mg).

¹H NMR (400 MHz, CDCl₃) δ 9.71 (s, 1H), 9.56 (d, J=7.7 Hz, 1H), 4.28-4.17 (m, 1H), 2.45 (tt, J=8.3, 4.9 Hz, 1H), 2.22-2.12 (m, 4H), 2.02-1.94 (m, 2H), 1.81-1.72 (m, 2H), 1.51-1.41 (m, 2H), 1.33-1.26 (m, 2H)

LCMS (Method 6): 1.26 min, 347.2 [M+H]⁺

### Synthesis of Compound 156, 7-cyclopropyl-N4-(4,4-difluorocyclohexyl)pyrazolo[5,1-c][1,2,4]triazine-4,8-dicarboxamide

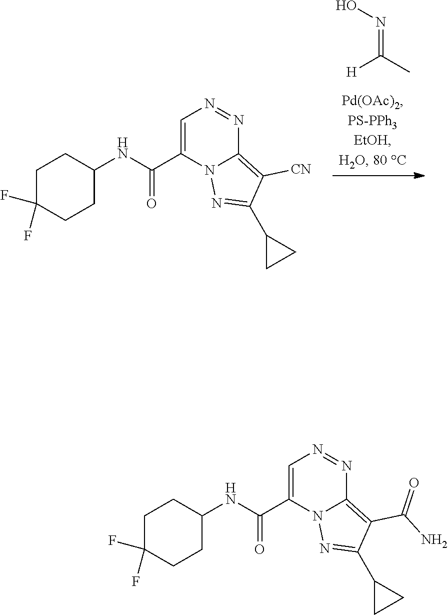

8-Cyano-7-cyclopropyl-N-(4,4-difluorocyclohexyl)pyrazolo[5,1-c][1,2,4]triazine-4-carboxamide (80 mg, 0.22 mmol), acetaldehyde oxime (70 μL, 1.10 mmol), Pd(OAc)₂ (9.9 mg, 0.04 mmol), and PS-PPh₃ (3 mmol/g) (20 mg, 0.06 mmol) were suspended in EtOH (800 μL) and water (200 μL), and the reaction was heated at 80° C. for 2 h. The reaction mixture was cooled to room temperature and concentrated in vacuo. The crude product was purified by column chromatography (eluting with 0 to 10% MeOH in DCM) and triturated with a 1:2 mixture of DMSO:water to provide the title compound (13 mg).

¹H NMR (400 MHz, DMSO-d₆) δ 9.41 (d, J=7.3 Hz, 1H), 9.37 (s, 1H), 7.89 (s, 1H), 7.85 (s, 1H), 4.17-4.10 (m, 1H), 3.30-3.25 (m, 1H), 2.13-1.94 (m, 6H), 1.78-1.65 (m, 2H), 1.31-1.22 (m, 2H), 1.15-1.05 (m, 2H)

LCMS (Method 7): 2.38 min, 365.2 [M+H]⁺

### Synthesis of Compound 157, 2-(trideuteriomethoxymethyl)-N7-[cis-3-(trifluoromethoxy)cyclobutyl]pyrazolo[1,5-a]pyrimidine-3,7-dicarboxamide (General Method O)

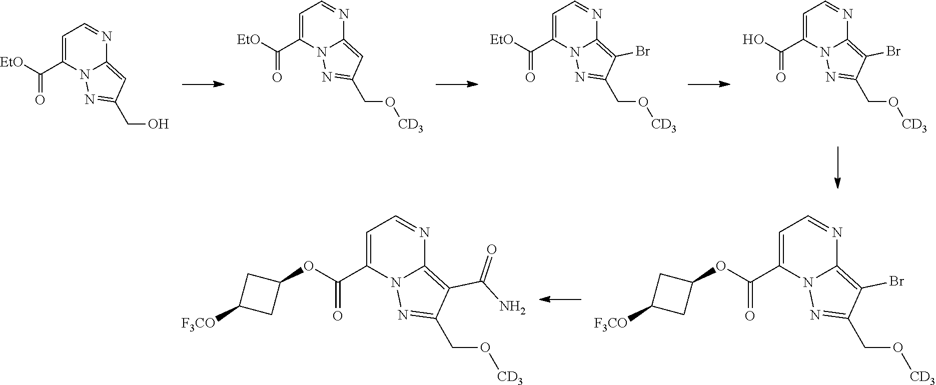 [SMILES: CCOC(=O)c1ccnc2cc(CO)nn12 ; [2H]C([2H])([2H])OCc1cc2nccc(C(=O)OCC)n2n1 ; [2H]C([2H])([2H])OCc1nn2c(C(=O)OCC)ccnc2c1Br ; [2H]C([2H])([2H])OCc1nn2c(C(=O)O)ccnc2c1Br ; [2H]C([2H])([2H])OCc1nn2c(C(=O)O[C@H]3C[C@@H](OC(F)(F)F)C3)ccnc2c1C(N)=O ; [2H]C([2H])([2H])OCc1nn2c(C(=O)O[C@H]3C[C@@H](OC(F)(F)F)C3)ccnc2c1Br]

### Synthesis of ethyl 2-(trideuteriomethoxymethyl)pyrazolo[1,5-a]pyrimidine-7-carboxylate

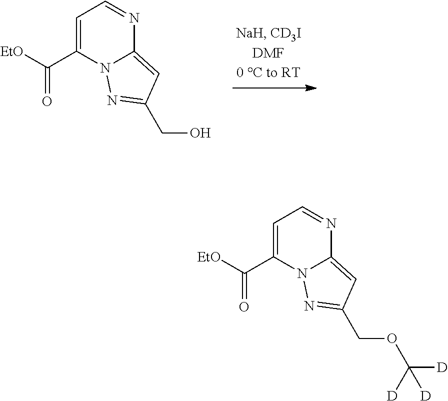 [SMILES: CCOC(=O)c1ccnc2cc(CO)nn12 ; [2H]C([2H])([2H])OCc1cc2nccc(C(=O)OCC)n2n1]

NaH (109 mg, 2.71 mmol) and trideuterio (iodo) methane (675 μL, 10.85 mmol) were added to a solution of ethyl 2-(hydroxymethyl)pyrazolo[1,5-a]pyrimidine-7-carboxylate (400 mg, 1.81 mmol) in DMF (18 mL) at 0° C. The reaction was warmed to room temperature and stirred for 18 h. The reaction mixture was diluted with EtOAc and water, and the layers were separated. The aqueous layer was acidified with 1 M aq. HCl and extracted with EtOAc. The combined organic layers were washed with sat. aq. LiCl, dried over Na₂SO₄, and concentrated in vacuo. The crude product was purified by column chromatography (eluting with 0 to 10% MeOH in DCM) to provide the title compound (183 mg).

¹H NMR (400 MHz, CDCl₃) δ 8.57-8.49 (m, 1H), 7.30-7.22 (m, 1H), 6.89-6.84 (m, 1H), 4.75 (s, 2H), 4.53 (q, J=7.2 Hz, 2H), 1.44 (t, J=7.1 Hz, 3H)

LCMS (Method 6): 1.55 min, 239.2 [M+H]⁺

### Synthesis of ethyl 3-bromo-2-(trideuteriomethoxymethyl)pyrazolo[1,5-a]pyrimidine-7-carboxylate

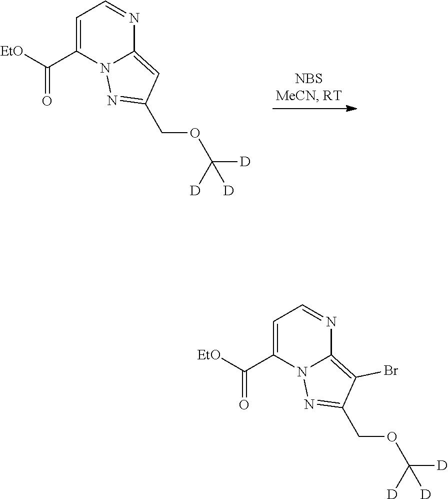 [SMILES: [2H]C([2H])([2H])OCc1cc2nccc(C(=O)OCC)n2n1 ; [2H]C([2H])([2H])OCc1nn2c(C(=O)OCC)ccnc2c1Br]

Ethyl 2-(trideuteriomethoxymethyl)pyrazolo[1,5-a]pyrimidine-7-carboxylate (180 mg, 0.76 mmol) and NBS (135 mg, 0.76 mmol) were dissolved in MeCN (4 mL) and the reaction was stirred at room temperature for 16 h. The reaction mixture was concentrated in vacuo and dissolved in DCM. The solution was washed with water, dried over Na₂SO₄, and concentrated in vacuo. The crude product was purified by column chromatography (eluting with 0 to 5% MeOH in DCM) to provide the title compound (247 mg).

¹H NMR (400 MHz, CDCl₃) δ 8.63 (d, J=4.1 Hz, 1H), 7.33 (d, J=4.1 Hz, 1H), 4.76 (d, J=0.7 Hz, 2H), 4.55 (q, J=7.1 Hz, 2H), 1.45 (t, J=7.1 Hz, 3H)

LCMS (Method 6): 1.77 min, 317.2, 319.2 [M+H]⁺

### Synthesis of 3-bromo-2-(trideuteriomethoxymethyl)pyrazolo[1,5-a]pyrimidine-7-carboxylic Acid

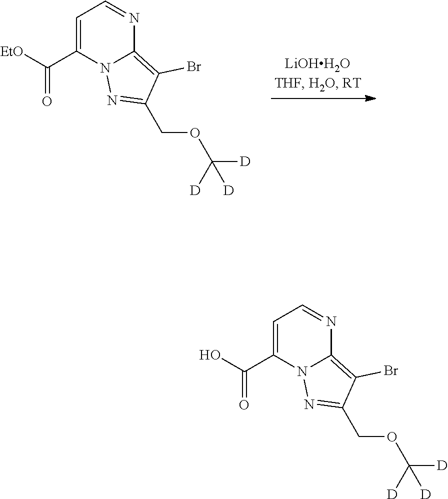 [SMILES: [2H]C([2H])([2H])OCc1nn2c(C(=O)OCC)ccnc2c1Br ; [2H]C([2H])([2H])OCc1nn2c(C(=O)O)ccnc2c1Br]

A solution of LiOH·H₂O (39 mg, 0.93 mmol) in water (1 mL) was added dropwise to a solution of ethyl 3-bromo-2-(trideuteriomethoxymethyl)pyrazolo[1,5-a]pyrimidine-7-carboxylate (245 mg, 0.77 mmol) in THF (2 mL) and the reaction was stirred at room temperature for 2 h. The reaction mixture was concentrated in vacuo, diluted with EtOAc, and acidified to pH 4 with 1 M aq. HCl. The layers were separated, and the organic layer was washed with brine, dried over Na₂SO₄, and concentrated in vacuo to provide the title compound (200 mg).

¹H NMR (400 MHz, DMSO-d₆) δ 8.74 (d, J=4.2 Hz, 1H), 7.48 (d, J=4.2 Hz, 1H), 4.61 (s, 2H). Exchangeable proton not observed.

LCMS (Method 6): 1.34 min, 289.2, 291.2 [M+H]⁺

### Synthesis of 3-bromo-2-(trideuteriomethoxymethyl)-N-[cis-3-(trifluoromethoxy)cyclobutyl]pyrazolo[1,5-a]pyrimidine-7-carboxamide

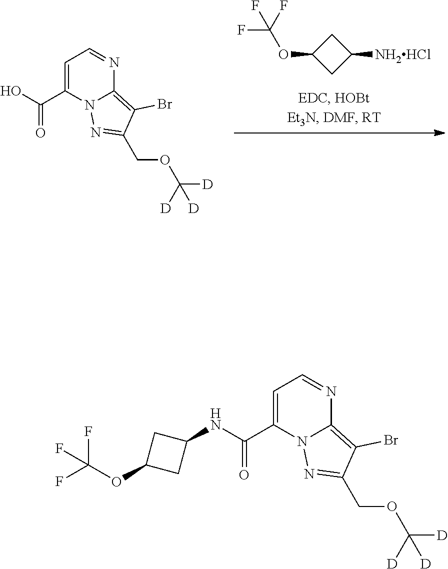

Cis-3-(trifluoromethoxy)cyclobutanamine hydrochloride (133 mg, 0.69 mmol), HOBt (140 mg, 1.04 mmol), EDC (199 mg, 1.04 mmol), and Et₃N (290 μL, 2.08 mmol) were added to a solution of 3-bromo-2-(trideuteriomethoxymethyl)pyrazolo[1,5-a]pyrimidine-7-carboxylic acid (100 mg, 0.35 mmol) in DMF (2 mL), and the reaction was stirred at room temperature for 16 h then diluted with EtOAc. The reaction mixture was washed with brine, dried through a hydrophobic frit, and concentrated in vacuo. The crude product was purified by column chromatography (eluting with 0 to 10% MeOH in DCM) to provide the title compound (120 mg).

¹H NMR (400 MHz, CDCl₃) δ 10.47 (d, J=6.9 Hz, 1H), 8.74 (d, J=4.2 Hz, 1H), 7.83 (d, J=4.2 Hz, 1H), 4.77 (s, 2H), 4.59-4.47 (m, 1H), 4.33-4.24 (m, 1H), 3.07-2.99 (m, 2H), 2.44-2.36 (m, 2H)

LCMS (Method 6): 2.12 min, 426.0, 428.0 [M+H]⁺

### Synthesis of Compound 157, 2-(trideuteriomethoxymethyl)-N7-[cis-3-(trifluoromethoxy)cyclobutyl]pyrazolo[1,5-a]pyrimidine-3,7-dicarboxamide

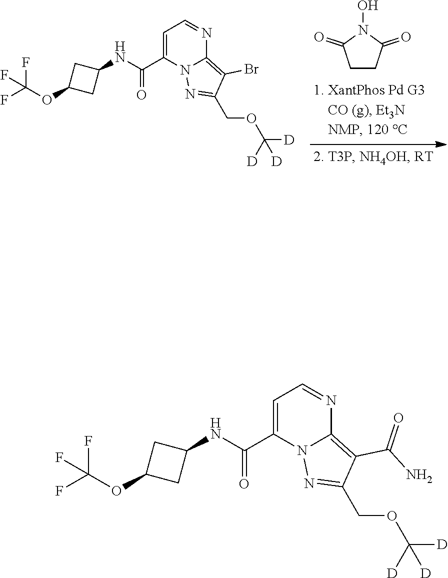 [SMILES: [2H]C([2H])([2H])OCc1nn2c(C(=O)N[C@H]3C[C@@H](OC(F)(F)F)C3)ccnc2c1Br ; O=C1CCC(=O)N1O ; [2H]C([2H])([2H])OCc1nn2c(C(=O)N[C@H]3C[C@@H](OC(F)(F)F)C3)ccnc2c1C(N)=O]

3-Bromo-2-(trideuteriomethoxymethyl)-N-[cis-3-(trifluoromethoxy)cyclobutyl]pyrazolo[1,5-a]pyrimidine-7-carboxamide (120 mg, 0.28 mmol), XantPhos Pd G3 (27 mg, 0.03 mmol), N-hydroxysuccinimide (49 mg, 0.42 mmol), and Et₃N (118 μL, 0.84 mmol) were dissolved in NMP (2 mL) and the solution was degassed by sparging with a stream of argon for 30 min. The reaction vial was sealed, evacuated, and backfilled with argon (×3), then evacuated and backfilled with carbon monoxide (×3). The reaction was heated at 120° C. in a microwave reactor for 4 h then cooled to room temperature. The reaction mixture was degassed with argon, then 1-propanephosphonic anhydride (50 wt % in EtOAc) (503 μL, 0.84 mmol) and NH₄OH (332 μL, 2.82 mmol) were added. The reaction was stirred at room temperature for 24 h, then diluted with EtOAc and washed with brine. The organic layer was dried over Na₂SO₄ and concentrated in vacuo. The crude product was purified by column chromatography (eluting with 0 to 10% MeOH in DCM) and preparative HPLC (acidic method) to provide the title compound (15 mg).

¹H NMR (400 MHz, DMSO-d₆) δ 10.05 (d, J=7.4 Hz, 1H), 8.91 (d, J=4.4 Hz, 1H), 7.68 (s, 1H), 7.62 (d, J=4.4 Hz, 1H), 7.59 (s, 1H), 4.99 (s, 2H), 4.77-4.66 (m, 1H), 4.28-4.13 (m, 1H), 2.96-2.85 (m, 2H), 2.36-2.25 (m, 2H)

LCMS (Method 7): 2.39 min, 391.2 [M+H]⁺

### Synthesis of Compound 160, N7-(6,7-dihydro-5H-cyclopenta[b]pyridin-6-yl)-2-(trideuteriomethoxymethyl)pyrazolo[1,5-a]pyrimidine-3,7-dicarboxamide

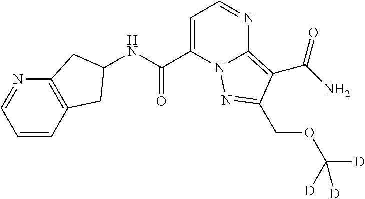 [SMILES: [2H]C([2H])([2H])OCc1nn2c(C(=O)NC3Cc4cccnc4C3)ccnc2c1C(N)=O]

N7-(6,7-dihydro-5H-cyclopenta[b]pyridin-6-yl)-2-(trideuteriomethoxymethyl)pyrazolo[1,5-a]pyrimidine-3,7-dicarboxamide (7 mg) was synthesised following a similar procedure to that described in General Method O.

¹H NMR (400 MHz, DMSO-d₆) δ 10.21 (d, J=7.0 Hz, 1H), 8.91 (d, J=4.4 Hz, 1H), 8.35 (dd, J=5.0, 1.6 Hz, 1H), 7.73-7.65 (m, 3H), 7.60-7.55 (m, 1H), 7.19 (dd, J=7.6, 4.9 Hz, 1H), 4.92 (s, 2H), 4.90-4.78 (m, 1H), 3.45 (dd, J=16.8, 7.6 Hz, 2H), 3.01 (dd, J=16.7, 4.5 Hz, 2H)

LCMS (Method 7): 1.37 min, 370.2 [M+H]⁺

### Synthesis of Compound 158, 2-(fluoromethoxymethyl)-N7-[cis-3-(trifluoromethoxy)cyclobutyl]pyrazolo[1,5-a]pyrimidine-3,7-dicarboxamide (General Method P)

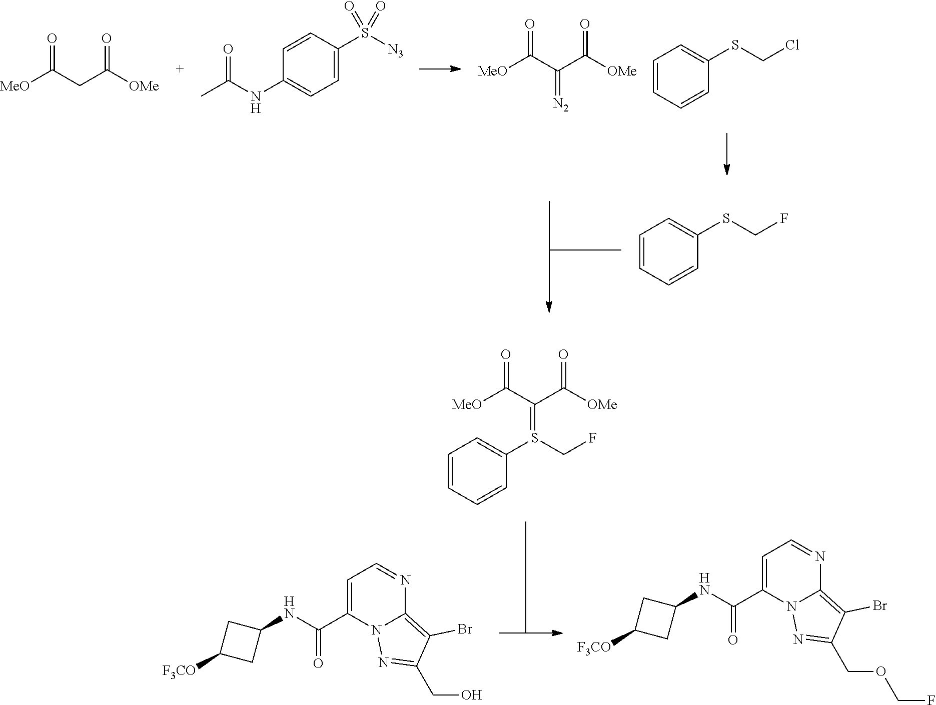

### Synthesis of Fluoromethylsulfanylbenzene

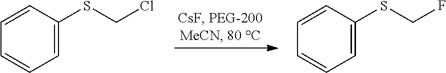 [SMILES: ClCSc1ccccc1 ; FCSc1ccccc1]

Chloromethylsulfanylbenzene (5 mL, 38.80 mmol) and CsF (11.78 g, 77.50 mmol) were suspended in MeCN (15 mL) and PEG-200 (5 mL, 38.80 mmol) and the reaction was heated at 80° C. for 3 h. The reaction mixture was cooled to room temperature and diluted with Et₂O and water. The layers were separated, and the organic layer was washed with water (×4) and brine, dried through a hydrophobic frit, and concentrated in vacuo. The crude product was purified by column chromatography (eluting with 0 to 100% DCM in heptane) to provide the title compound (2.9 g).

¹H NMR (400 MHz, CDCl₃) δ 7.51-7.48 (m, 2H), 7.36-7.30 (m, 3H), 5.73 (d, J=52.8 Hz, 2H)

### Synthesis of dimethyl 2-diazopropanedioate

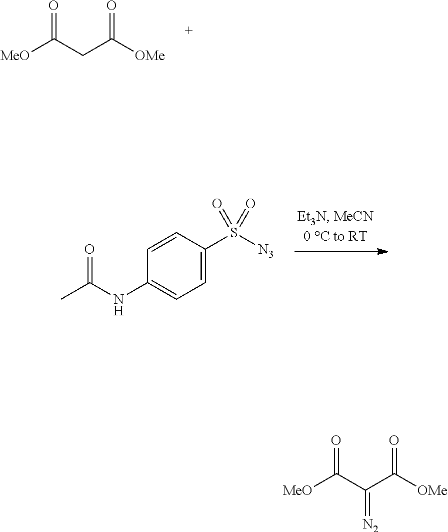

Dimethyl malonate (2 mL, 17.56 mmol) was added to a solution of N-(4-azidosulfonylphenyl) acetamide (5 g, 20.81 mmol) and Et₃N (4.9 mL, 35.12 mmol) in MeCN (50 mL) at 0° C. The reaction was warmed to room temperature and stirred for 18 h. The reaction mixture was diluted with Et₂O, filtered, and concentrated in vacuo. The crude product was triturated with heptane to provide the title compound (2.3 g).

¹H NMR (400 MHz, CDCl₃) δ 3.84 (s, 6H)

### Synthesis of dimethyl 2-[fluoromethyl(phenyl)-λ4-sulfanylidene]propanedioate

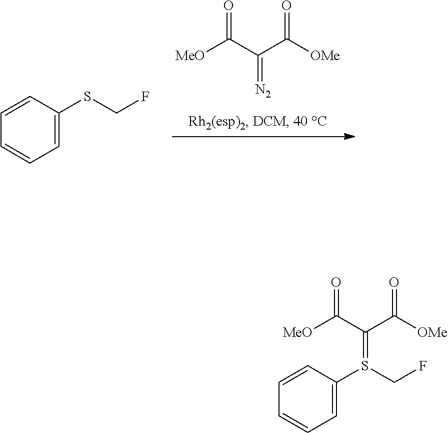

A solution of dimethyl 2-diazopropanedioate (2.35 g, 14.86 mmol) in DCM (30 mL) was added to a solution of fluoromethylsulfanylbenzene (2.11 g, 14.86 mmol) and bis[rhodium (α,α,α′,α′-tetramethyl-1,3-benzenedipropionic acid)] (15 mg, 0.02 mmol) in DCM (50 mL). The reaction was heated at 40° C. for 23 h, then cooled to room temperature and concentrated in vacuo. The crude product was purified by column chromatography (eluting with 0 to 100% EtOAc in heptane) to provide the title compound (2.9 g).

¹H NMR (400 MHz, CDCl₃) δ 7.68-7.60 (m, 2H), 7.60-7.42 (m, 3H), 6.41 (dd, J=50.5, 7.6 Hz, 1H), 5.86 (dd, J=45.3, 7.6 Hz, 1H), 3.71 (s, 6H)

LCMS (Method 3) δ 0.93 min, 273.2 [M+H]⁺

### Synthesis of 3-bromo-2-(fluoromethoxymethyl)-N-[cis-3-(trifluoromethoxy)cyclobutyl]pyrazolo[1,5-a]pyrimidine-7-carboxamide

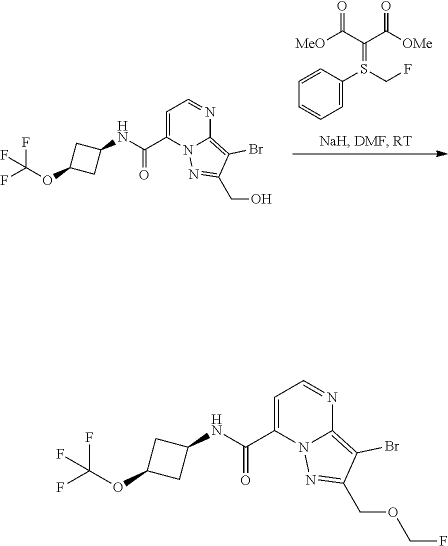 [SMILES: O=C(N[C@H]1C[C@@H](OC(F)(F)F)C1)c1ccnc2c(Br)c(CO)nn12 ; COC(=O)C(C(=O)OC)=S(CF)c1ccccc1 ; O=C(N[C@H]1C[C@@H](OC(F)(F)F)C1)c1ccnc2c(Br)c(COCF)nn12]

NaH (60 wt % in mineral oil) (87 mg, 2.18 mmol) was added to a solution of 3-bromo-2-(hydroxymethyl)-N-[cis-3-(trifluoromethoxy)cyclobutyl]pyrazolo[1,5-a]pyrimidine-7-carboxamide (445 mg, 1.09 mmol) in DMF (10 mL) and the mixture was stirred at room temperature for 30 min. Dimethyl 2-[fluoromethyl(phenyl)-λ4-sulfanylidene]propanedioate (296 mg, 1.09 mmol) was added and the reaction was stirred at room temperature for 18 h. The reaction mixture was diluted with EtOAc and water, and the layers were separated. The organic layer was washed with water (×3) and brine, dried over Na₂SO₄, and concentrated in vacuo. The crude product was purified by column chromatography (eluting with 0 to 50% EtOAc in heptane) to provide the title compound (200 mg).

¹H NMR (400 MHz, DMSO-d₆) δ 10.41 (d, J=6.9 Hz, 1H), 8.76 (d, J=4.2 Hz, 1H), 7.86 (d, J=4.2 Hz, 1H), 5.54 (s, 1H), 5.42 (d, J=14.2 Hz, 1H), 5.10 (d, J=1.7 Hz, 2H), 4.59-4.48 (m, 1H), 4.38-4.23 (m, 1H), 3.09-2.97 (m, 2H), 2.46-2.34 (m, 2H)

LCMS (Method 7): 3.03 min, 441.0, 443.0 [M+H]⁺

### Synthesis of Compound 158, 2-(fluoromethoxymethyl)-N7-[cis-3-(trifluoromethoxy)cyclobutyl]pyrazolo[1,5-a]pyrimidine-3,7-dicarboxamide

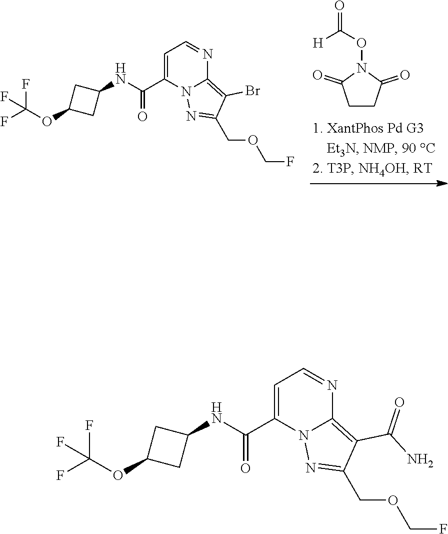 [SMILES: O=C(N[C@H]1C[C@@H](OC(F)(F)F)C1)c1ccnc2c(Br)c(COCF)nn12 ; O=CON1C(=O)CCC1=O ; NC(=O)c1c(COCF)nn2c(C(=O)N[C@H]3C[C@@H](OC(F)(F)F)C3)ccnc12]

3-Bromo-2-(fluoromethoxymethyl)-N-[cis-3-(trifluoromethoxy)cyclobutyl]pyrazolo[1,5-a]pyrimidine-7-carboxamide (100 mg, 0.23 mmol) and XantPhos Pd G3 (22 mg, 0.02 mmol) were placed in a vial and the vial was sealed and purged with argon for 5 min. NMP (2 mL) and Et₃N (95 μL, 0.68 mmol) were added and the solution was degassed by sparging with a stream of argon for 5 min. (2,5-Dioxopyrrolidin-1-yl) formate (162 mg, 1.13 mmol) was added and the vial was sealed immediately. The reaction was heated at 90° C. in a microwave reactor for 2 h. Additional portions of XantPhos Pd G3 (22 mg, 0.01 mmol) and Et₃N (95 μL, 0.68 mmol) were added and the mixture was purged with argon for 5 min. An additional portion of (2,5-dioxopyrrolidin-1-yl) formate (162 mg, 1.13 mmol) was added and the vial was sealed immediately. The reaction was heated at 90° C. in a microwave reactor for a further 2 h. The reaction mixture was cooled to room temperature, and NH₄OH (295 μL, 2.97 mmol) and 1-propanephosphonic anhydride (50 wt % in DMF) (240 μL, 0.45 mmol) were added. The reaction was stirred at room temperature for 18 h then quenched with water. The resulting precipitate was collected by filtration, washed with water, and dried. The crude product was purified by column chromatography (eluting with 0 to 3% MeOH in DCM) to provide the title compound (29 mg).

¹H NMR (400 MHz, DMSO-d₆) δ 9.98 (d, J=7.4 Hz, 1H), 8.94 (d, J=4.3 Hz, 1H), 7.70-7.62 (m, 3H), 5.52 (d, J=56.5 Hz, 2H), 5.33 (d, J=1.9 Hz, 2H), 4.77-4.65 (m, 1H), 4.28-4.13 (m, 1H), 2.95-2.83 (m, 2H), 2.38-2.28 (m, 2H)

LCMS (Method 7): 2.53 min, 406.1 [M+H]⁺

### Synthesis of Compound 159, N7-indan-2-yl-2-(methylamino)pyrazolo[1,5-a]pyrimidine-3,7-dicarboxamide (General Method Q)

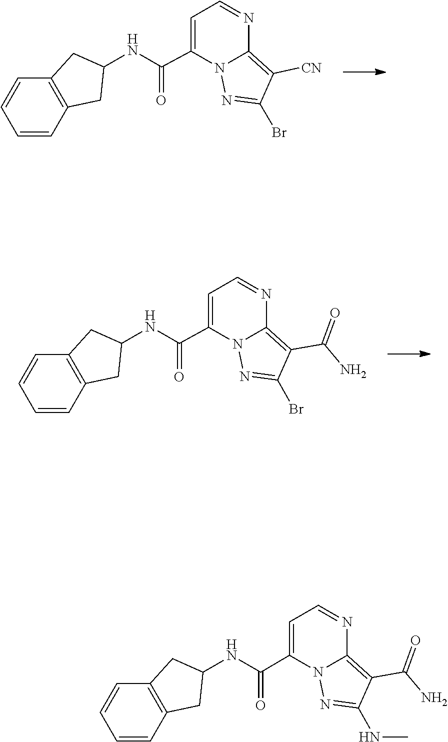 [SMILES: N#Cc1c(Br)nn2c(C(=O)NC3Cc4ccccc4C3)ccnc12 ; NC(=O)c1c(Br)nn2c(C(=O)NC3Cc4ccccc4C3)ccnc12 ; CNc1nn2c(C(=O)NC3Cc4ccccc4C3)ccnc2c1C(N)=O]

### Synthesis of 2-bromo-N7-indan-2-yl-pyrazolo[1,5-a]pyrimidine-3,7-dicarboxamide

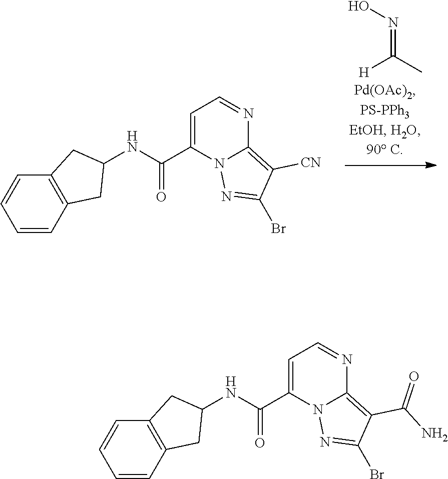

2-Bromo-3-cyano-N-indan-2-yl-pyrazolo[1,5-a]pyrimidine-7-carboxamide (200 mg, 0.52 mmol), acetaldehyde oxime (128 μL, 2.09 mmol), and PS-PPh₃ (1.4-2 mmol/g) (113 mg, 0.16-0.23 mmol) were suspended in EtOH (4.1 mL) and water (870 μL), and the mixture was sparged with argon for 10 min. Pd(OAc)₂ (18 mg, 0.08 mmol) was added, and the suspension was sparged with argon for a further 10 min. The reaction was heated at 90° C. in a microwave reactor for 3 h, then cooled to room temperature, diluted with DCM, and filtered through a short pad of Dicalite®. The filtrate was concentrated in vacuo, and the crude product was purified by column chromatography (eluting with 0 to 5% MeOH in DCM) to provide the title compound (86 mg).

¹H NMR (400 MHz, DMSO-d₆) δ 9.62 (d, J=7.1 Hz, 1H), 8.91 (d, J=4.4 Hz, 1H), 7.67 (d, J=5.9 Hz, 2H), 7.57 (d, J=4.4 Hz, 1H), 7.32-7.25 (m, 2H), 7.22-7.15 (m, 2H), 4.82-4.73 (m, 1H), 3.38 (dd, J=16.0, 7.7 Hz, 2H), 2.96 (dd, J=16.1, 5.1 Hz, 2H)

LCMS (Method 6): 1.88 min, 400.0, 402.0 [M+H]⁺

### Synthesis of Compound 159, N7-indan-2-yl-2-(methylamino)pyrazolo[1,5-a]pyrimidine-3,7-dicarboxamide

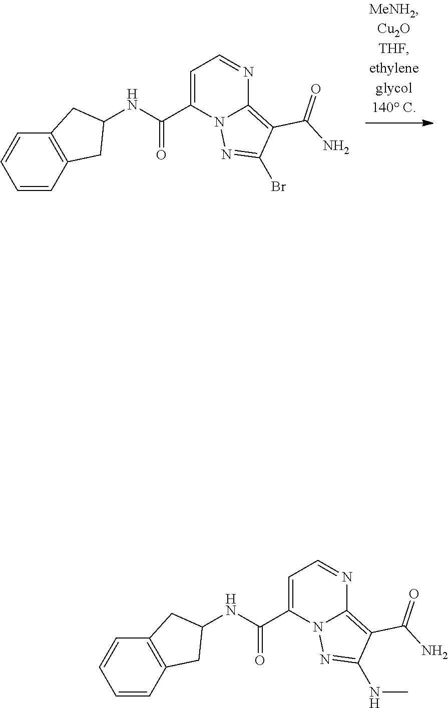 [SMILES: NC(=O)c1c(Br)nn2c(C(=O)NC3Cc4ccccc4C3)ccnc12 ; CNc1nn2c(C(=O)NC3Cc4ccccc4C3)ccnc2c1C(N)=O]

2-Bromo-N7-indan-2-yl-pyrazolo[1,5-a]pyrimidine-3,7-dicarboxamide (83 mg, 0.21 mmol), Cu₂O (3 mg, 0.02 mmol), and MeNH₂ (2 M in THF) (3.1 mL, 6.22 mmol) were suspended in ethylene glycol (830 μL) and the reaction was heated at 140° C. in a microwave reactor for 1 h. The reaction mixture was cooled to room temperature, filtered through a short pad of Dicalite® and concentrated in vacuo. The crude product was purified by column chromatography (eluting with 0 to 5% MeOH in DCM) then triturated with Et₂O to provide the title compound (17 mg).

¹H NMR (400 MHz, DMSO-d₆) δ 10.64 (d, J=7.6 Hz, 1H), 8.63 (d, J=4.7 Hz, 1H), 7.49 (d, J=4.7 Hz, 1H), 7.37 (s, 1H), 7.33-7.26 (m, 3H), 7.21-7.15 (m, 2H), 6.92 (q, J=5.0 Hz, 1H), 4.87-4.76 (m, 1H), 3.36 (dd, J=16.1, 6.5 Hz, 2H), 2.95 (dd, J=16.1, 3.3 Hz, 2H), 2.58 (d, J=5.0 Hz, 3H)

LCMS (Method 7): 2.64 min, 351.2 [M+H]⁺

### Synthesis of Compound 161, 2-(methoxymethyl)-N3-methyl-N7-[cis-3-(trifluoromethoxy)cyclobutyl]pyrazolo[1,5-a]pyrimidine-3,7-dicarboxamide (General Method R)

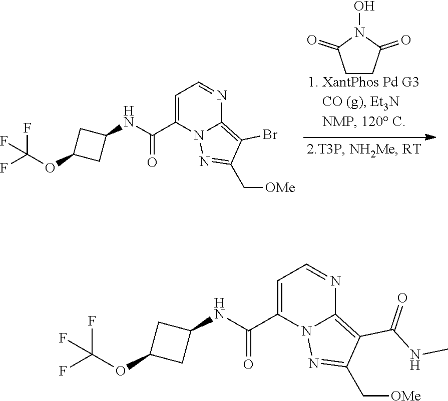 [SMILES: O=C1CCC(=O)N1O ; COCc1nn2c(C(=O)N[C@H]3C[C@@H](OC(F)(F)F)C3)ccnc2c1Br ; CNC(=O)c1c(COC)nn2c(C(=O)N[C@H]3C[C@@H](OC(F)(F)F)C3)ccnc12]

3-Bromo-2-(methoxymethyl)-N-[cis-3-(trifluoromethoxy)cyclobutyl]pyrazolo[1,5-a]pyrimidine-7-carboxamide (120 mg, 0.28 mmol), XantPhos Pd G3 (22 mg, 0.03 mmol), N-hydroxysuccinimide (41 mg, 0.35 mmol), and Et₃N (99 μL, 0.71 mmol) were dissolved in NMP (2 mL), and the solution was degassed by sparging with a stream of argon for 30 min. The reaction vial was sealed, evacuated and backfilled with argon (×3), then evacuated and backfilled with carbon monoxide (×3). The reaction was heated at 120° C. in a microwave reactor for 4 h then cooled to room temperature. The reaction mixture was degassed with argon, then 1-propanephosphonic anhydride (50 wt % in EtOAc) (422 μL, 0.71 mmol) and NH₂Me (2 M in THF) (1.18 mL, 2.36 mmol) were added. The reaction was stirred at room temperature for 24 h, then diluted with EtOAc and washed with brine. The organic layer was dried over Na₂SO₄ and concentrated in vacuo. The crude product was purified by column chromatography (eluting with 0 to 10% MeOH in DCM) and preparative HPLC (acidic method) to provide the title compound (29 mg).

¹H NMR (400 MHz, DMSO-d₆) δ 10.04 (d, J=7.4 Hz, 1H), 8.91 (d, J=4.4 Hz, 1H), 8.12-8.06 (m, 1H), 7.62 (d, J=4.3 Hz, 1H), 4.99 (s, 2H), 4.77-4.66 (m, 1H), 4.27-4.13 (m, 1H), 3.42 (s, 3H), 2.96-2.85 (m, 5H), 2.36-2.25 (m, 2H)

LCMS (Method 7): 2.53 min, 402.2 [M+H]⁺

### Synthesis of Compound 162, 2-(cyclopropylmethoxy)-N7-[cis-3-(trifluoromethoxy)cyclobutyl]pyrazolo[1,5-a]pyrimidine-3,7-dicarboxamide (General Method S)

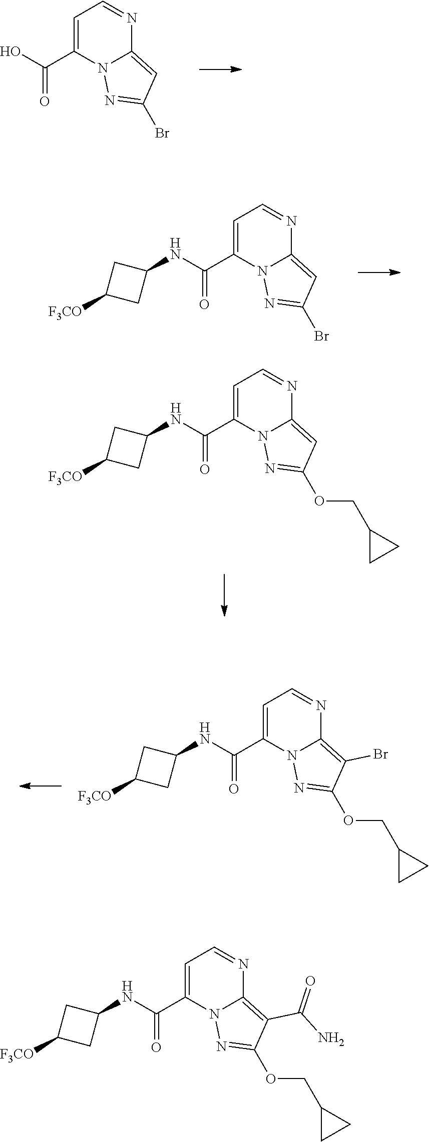 [SMILES: O=C(O)c1ccnc2cc(Br)nn12 ; O=C(N[C@H]1C[C@@H](OC(F)(F)F)C1)c1ccnc2cc(Br)nn12 ; O=C(N[C@H]1C[C@@H](OC(F)(F)F)C1)c1ccnc2cc(OCC3CC3)nn12 ; O=C(N[C@H]1C[C@@H](OC(F)(F)F)C1)c1ccnc2c(Br)c(OCC3CC3)nn12 ; NC(=O)c1c(OCC2CC2)nn2c(C(=O)N[C@H]3C[C@@H](OC(F)(F)F)C3)ccnc12]

### Synthesis of 2-bromo-N-[cis-3-(trifluoromethoxy)cyclobutyl]pyrazolo[1,5-a]pyrimidine-7-carboxamide

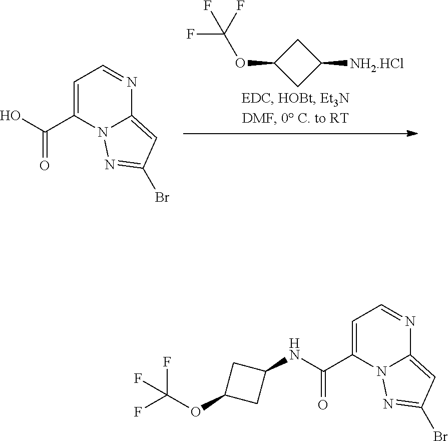

Et₃N (3.46 mL, 24.79 mmol) was added to a solution of 2-bromopyrazolo[1,5-a]pyrimidine-7-carboxylic acid (1 g, 4.13 mmol), cis-3-(trifluoromethoxy)cyclobutanamine hydrochloride (950 mg, 4.96 mmol), HOBt (2.37 g, 12.4 mmol), and EDC (2.38 g, 12.4 mmol) in DMF (21 mL) at 0° C. and the reaction was stirred at 0° C. for 5 min then warmed to room temperature and stirred for 16 h. The reaction mixture was diluted with EtOAc and washed with 5% aq. LiCl (×3) and brine. The organic layer was dried over Na₂SO₄ and concentrated in vacuo. The crude product was purified by column chromatography (eluting with 0 to 40% EtOAc in heptane) to provide the title compound (1.44 g).

¹H NMR (400 MHz, CDCl₃) δ 10.30 (br. d, J=6.5 Hz, 1H), 8.68 (d, J=4.3 Hz, 1H), 7.77 (d, J=4.3 Hz, 1H), 6.93 (s, 1H), 4.53 (p, J=7.2 Hz, 1H), 4.37-4.23 (m, 1H), 3.10-2.98 (m, 2H), 2.48-2.36 (m, 2H)

LCMS (Method 7): 3.07 min, 379.0, 381.0 [M+H]⁺

### Synthesis of 2-(cyclopropylmethoxy)-N-[cis-3-(trifluoromethoxy)cyclobutyl]pyrazolo[1,5-a]pyrimidine-7-carboxamide

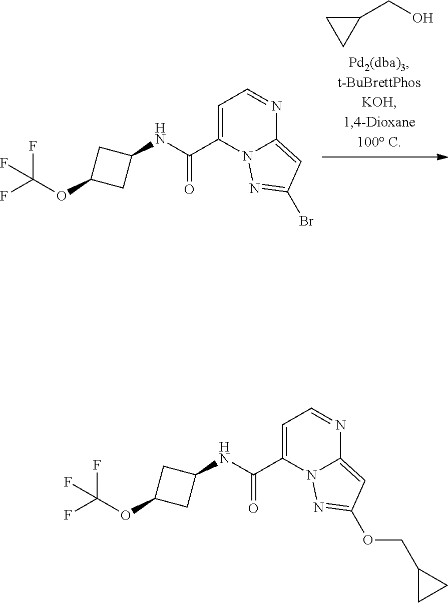 [SMILES: OCC1CC1 ; O=C(N[C@H]1C[C@@H](OC(F)(F)F)C1)c1ccnc2cc(Br)nn12 ; O=C(N[C@H]1C[C@@H](OC(F)(F)F)C1)c1ccnc2cc(OCC3CC3)nn12]

Cyclopropylmethanol (26.4 mL, 325.53 mmol) and KOH (296 mg, 5.28 mmol) were added to a solution of 2-bromo-N-[cis-3-(trifluoromethoxy)cyclobutyl]pyrazolo[1,5-a]pyrimidine-7-carboxamide (1 g, 2.64 mmol) in 1,4-dioxane (26.4 mL) and the solution was degassed by sparging with a stream of argon for 10 min. Pd₂ (dba) 3 (121 mg, 0.13 mmol) and t-BuBrettPhos (128 mg, 0.26 mmol) were added, and the solution was degassed with argon for a further 10 min. The reaction was heated at 100° C. for 16 h then cooled to room temperature, filtered through a pad of silica gel, and concentrated in vacuo. The residue was azeotroped with toluene (×3), and the crude product was purified by column chromatography (eluting with 0 to 50% EtOAc in heptane) to provide the title compound (481 mg).

¹H NMR (400 MHz, CDCl₃): δ 10.58 (br. d, J=7.1 Hz, 1H), 8.55 (d, J=4.4 Hz, 1H), 7.63 (d, J=4.4 Hz, 1H), 6.20 (s, 1H), 4.53 (p, J=7.2 Hz, 1H), 4.40-4.26 (m, 1H), 4.15 (d, J=7.2 Hz, 2H), 3.11-2.99 (m, 2H), 2.42-2.30 (m, 2H), 1.49-1.35 (m, 1H), 0.78-0.65 (m, 2H), 0.52-0.43 (m, 2H)

LCMS (Method 7): 3.19 min, 371.2 [M+H]⁺

### Synthesis of 3-bromo-2-(cyclopropylmethoxy)-N-[cis-3-(trifluoromethoxy)cyclobutyl]pyrazolo[1,5-a]pyrimidine-7-carboxamide

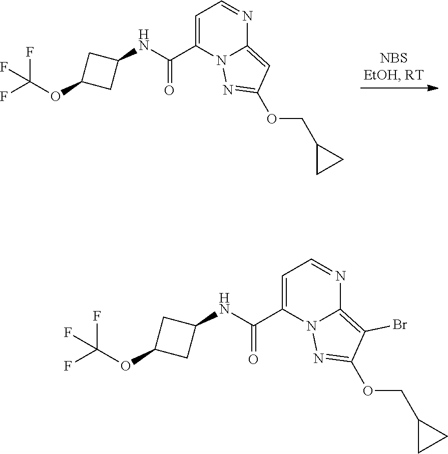 [SMILES: O=C(N[C@H]1C[C@@H](OC(F)(F)F)C1)c1ccnc2cc(OCC3CC3)nn12 ; O=C(N[C@H]1C[C@@H](OC(F)(F)F)C1)c1ccnc2c(Br)c(OCC3CC3)nn12]

NBS (277 mg, 1.56 mmol) was added to a suspension of 2-(cyclopropylmethoxy)-N-[cis-3-(trifluoromethoxy)cyclobutyl]pyrazolo[1,5-a]pyrimidine-7-carboxamide (480 mg, 1.30 mmol) in EtOH (26 mL) at 0° C. and the reaction was stirred for 5 min then warmed to room temperature and stirred for a further 10 min. The reaction was quenched with sat. aq. Na₂S₂O₃ and diluted with EtOAc. The layers were separated, and the aqueous layer was extracted with EtOAc (×4). The combined organic layers were washed with brine, dried over Na₂SO₄, and concentrated in vacuo. The crude product was purified by column chromatography (eluting with 0 to 40% EtOAc in heptane) to provide the title compound (508 mg).

¹H NMR (400 MHz, CDCl₃): δ 10.32 (d, J=7.2 Hz, 1H), 8.63 (d, J=4.4 Hz, 1H), 7.65 (d, J=4.4 Hz, 1H), 4.55 (p, J=7.1 Hz, 1H), 4.40-4.26 (m, 3H), 3.12-3.00 (m, 2H), 2.34-2.22 (m, 2H), 1.54-1.39 (m, 1H), 0.80-0.67 (m, 2H), 0.52-0.44 (m, 2H)

LCMS (Method 7): 3.40 min, 449.0, 451.0 [M+H]⁺

### Synthesis of Compound 162, 2-(cyclopropylmethoxy)-N7-[cis-3-(trifluoromethoxy)cyclobutyl]pyrazolo[1,5-a]pyrimidine-3,7-dicarboxamide

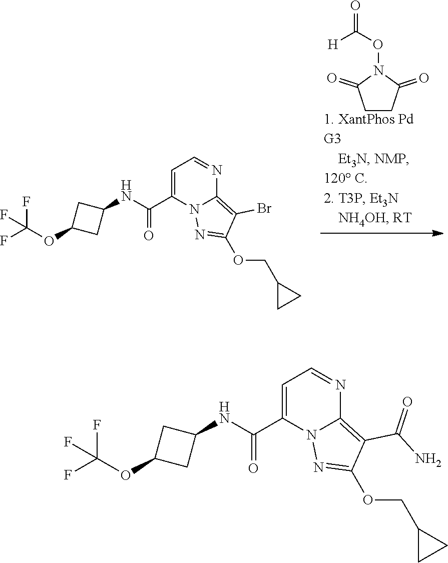 [SMILES: O=CON1C(=O)CCC1=O ; O=C(N[C@H]1C[C@@H](OC(F)(F)F)C1)c1ccnc2c(Br)c(OCC3CC3)nn12 ; NC(=O)c1c(OCC2CC2)nn2c(C(=O)N[C@H]3C[C@@H](OC(F)(F)F)C3)ccnc12]

Et₃N (470 μL, 3.34 mmol) was added to a vial containing a solution of 3-bromo-2-(cyclopropylmethoxy)-N-[cis-3-(trifluoromethoxy)cyclobutyl]pyrazolo[1,5-a]pyrimidine-7-carboxamide (500 mg, 1.11 mmol) in NMP (11 mL). The vial was sealed, and the solution was degassed by sparging with a stream of argon for 10 min. XantPhos Pd G3 (106 mg, 0.11 mmol) was added, and the solution was degassed with argon for a further 10 min. (2,5-Dioxopyrrolidin-1-yl) formate (796 mg, 5.57 mmol) was added, and the vial was sealed immediately. The reaction was heated at 120° C. in a microwave reactor for 2 h. Additional portions of XantPhos Pd G3 (106 mg, 0.11 mmol) and Et₃N (470 μL, 3.34 mmol) were added, and the mixture was purged with argon for 5 min. An additional portion of (2,5-dioxopyrrolidin-1-yl) formate (398 mg, 2.78 mmol) was added and the vial was sealed immediately. The reaction was heated at 120° C. in a microwave reactor for a further 1 h.

The reaction mixture was cooled to room temperature, and Et₃N (470 μL, 3.34 mmol), NH₄OH (1.44 mL, 11.1 mmol) and 1-propanephosphonic anhydride (50 wt % in DMF) (1.33 mL, 2.23 mmol) were added. The reaction was stirred at room temperature for 72 h, then quenched with water. The resulting precipitate was collected by filtration, washed with water, and purified by column chromatography (eluting with 0 to 10% MeOH in DCM) to provide the title compound (280 mg).

¹H NMR (400 MHz, DMSO-d₆): δ 9.74 (br. s, 1H), 8.78 (d, J=4.5 Hz, 1H), 7.47 (d, J=4.5 Hz, 1H), 7.42 (br. s, 1H), 7.32 (br. s, 1H), 4.70 (p, J=7.1 Hz, 1H), 4.30 (d, J=7.2 Hz, 2H), 4.25-4.14 (m, 1H), 2.95-2.84 (m, 2H), 2.37-2.25 (m, 2H), 1.50-1.36 (m, 1H), 0.65-0.55 (m, 2H), 0.45-0.37 (m, 2H)

LCMS (Method 7): 2.61 min, 414.2 [M+H]⁺

### Synthesis of Compound 212, N4-(4,4-difluorocyclohexyl)-6-(methoxymethyl)imidazo[2,1-f][1,2,4]triazine-4,7-dicarboxamide (General Method S)

### Synthesis of 4-chloro-6-hydrazino-pyrimidin-5-amine

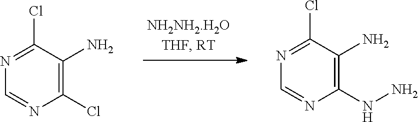 [SMILES: Nc1c(Cl)ncnc1Cl ; NNc1ncnc(Cl)c1N]

Hydrazine monohydrate (8.13 mL, 167.69 mmol) was added to a solution of 4,6-dichloropyrimidin-5-amine (5 g, 30.49 mmol) in THF (20 mL) and the reaction was stirred at room temperature for 20 h. The resulting precipitate was collected by filtration, washed with i-PrOH, and dried to provide the title compound (4.33 g).

¹H NMR (400 MHz, DMSO-d₆) δ 8.06 (s, 1H), 7.79 (s, 1H), 5.04 (s, 2H), 4.42 (s, 2H)

### Synthesis of 5-chloro-1,2-dihydropyrimido[5,4-e][1,2,4]triazine

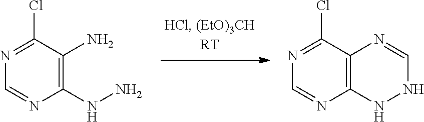 [SMILES: NNc1ncnc(Cl)c1N ; Clc1ncnc2c1N=CNN2]

Concentrated HCl (500 μL, 16.46 mmol) was added slowly to a suspension of 4-chloro-6-hydrazino-pyrimidin-5-amine (1 g, 6.27 mmol) in triethylorthoformate (11 mL, 66.06 mmol) and the reaction was stirred at room temperature for 1 h. The resulting precipitate was collected by filtration, washed with acetone, and dried to provide the title compound (895 mg).

¹H NMR (400 MHz, DMSO-d₆) δ 9.11 (s, 1H), 8.01 (s, 1H), 7.40 (s, 1H), 6.21 (s, 1H)

LCMS (Method 6): 0.77 min, 170.2 [M+H]⁺

### Synthesis of 5-methoxypyrimido[5,4-e][1,2,4]triazine

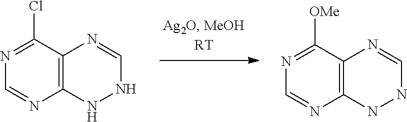 [SMILES: Clc1ncnc2c1N=CNN2 ; COc1ncnc2c1N=CNN2]

Silver oxide (2.73 g, 11.79 mmol) and 5-chloro-1,2-dihydropyrimido[5,4-e][1,2,4]triazine (1 g, 5.90 mmol) were suspended in MeOH (160 mL) and the reaction was stirred at room temperature for 66 h. The reaction mixture was filtered through a short pad of Dicalite® layered with silica gel and washed with EtOAc. The filtrate was concentrated in vacuo, and the crude product was purified by column chromatography (eluting with 0 to 100% EtOAc in heptane) to provide the title compound (296 mg).

¹H NMR (400 MHz, MeOD) δ 10.20 (s, 1H), 9.13 (s, 1H), 4.34 (s, 3H)

LCMS (Method 6): 0.63 min, 164.2 [M+H]⁺

### Synthesis of methyl 6-amino-1,2,4-triazine-5-carboxylate

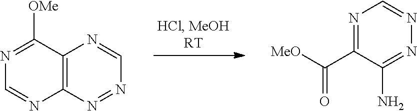 [SMILES: COc1ncnc2nncnc12 ; COC(=O)c1ncnnc1N]

1 M aq. HCl (2.5 mL, 69.04 mmol) was added to a solution of 5-methoxypyrimido[5,4-e][1,2,4]triazine (292 mg, 1.79 mmol) in MeOH (10 mL). The reaction was stirred at room temperature for 20 h then concentrated in vacuo. Water was added to the residue, and the mixture was basified to pH 8 with sat. aq. NaHCO₃ and diluted with DCM. The layers were separated, and the aqueous layer was extracted with DCM (×3). The combined organic layers were dried through a hydrophobic frit and concentrated in vacuo to provide the title compound (250 mg).

¹H NMR (400 MHz, DMSO-d₆) δ 9.17 (s, 1H), 7.55 (s, 2H), 3.91 (s, 3H)

LCMS (Method 6): 0.57 min, 155.3 [M+H]⁺

### Synthesis of 6-amino-N-(4,4-difluorocyclohexyl)-1,2,4-triazine-5-carboxamide

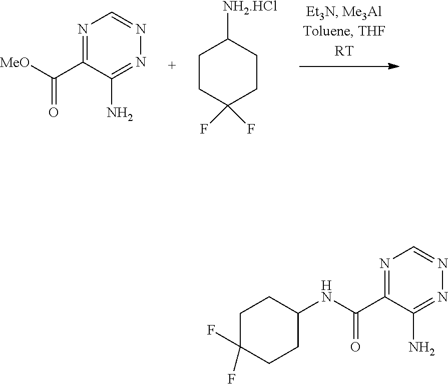

Et₃N (830 μL, 5.98 mmol) and Me₃Al (trimethylaluminium; 2 M in toluene) (4.98 mL, 9.97 mmol) were added sequentially to a suspension of 4,4-difluorocyclohexanamine hydrochloride (941 mg, 5.48 mmol) in THF (10 mL), and the reaction was stirred at room temperature for 30 min. A solution of methyl 6-amino-1,2,4-triazine-5-carboxylate (768 mg, 4.98 mmol) in THF (15 mL) was added dropwise and the reaction was stirred at room temperature for 5.5 h. The reaction mixture was poured into 10% aq. Rochelle salt and the mixture was stirred for 10 min then diluted with DCM. The layers were separated, and the aqueous layer was extracted with DCM. The combined organic layers were washed with brine, dried through a hydrophobic frit, and filtered through a short pad of silica gel. The filtrate was concentrated in vacuo to provide the title compound (972 mg).

¹H NMR (400 MHz, DMSO-d₆) δ 9.20-8.98 (m, 2H), 7.70 (s, 2H), 4.05-3.89 (m, 1H), 2.12-1.65 (m, 8H)

LCMS (Method 6): 1.57 min, 258.2 [M+H]⁺

### Synthesis of methyl 2-chloro-4-methoxy-3-oxo-butanoate

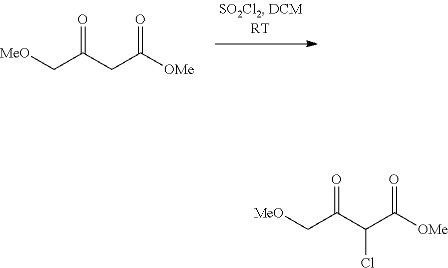 [SMILES: COCC(=O)CC(=O)OC ; COCC(=O)C(Cl)C(=O)OC]

Sulfuryl chloride (1.2 mL, 14.80 mmol) was added slowly to a solution of methyl 4-methoxy-3-oxo-butanoate (1.77 mL, 13.69 mmol) in DCM (20 mL) and the reaction was stirred at room temperature for 21 h. The reaction mixture was concentrated in vacuo, and the crude product was purified by column chromatography (eluting with 0 to 50% EtOAc in heptane) to provide the title compound (1.75 g).

¹H NMR (400 MHz, CDCl₃) δ 5.06 (s, 1H), 4.28 (d, J=1.5 Hz, 2H), 3.84 (s, 3H), 3.43 (s, 3H)

### Synthesis of methyl 4-[(4,4-difluorocyclohexyl) carbamoyl]-6-(methoxymethyl)imidazo[2,1-f][1,2,4]triazine-7-carboxylate

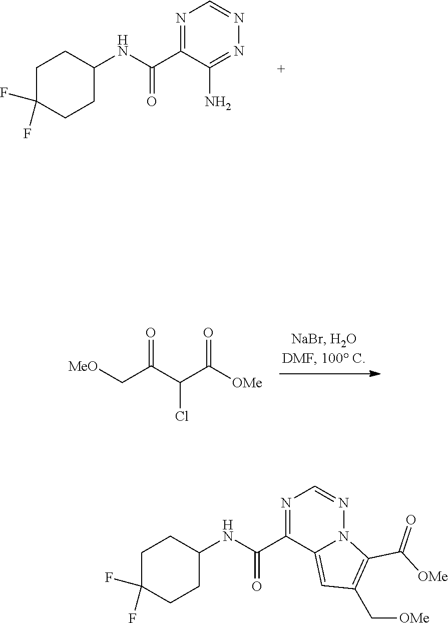 [SMILES: Nc1nncnc1C(=O)NC1CCC(F)(F)CC1 ; COCC(=O)C(Cl)C(=O)OC ; COCc1cc2c(C(=O)NC3CCC(F)(F)CC3)ncnn2c1C(=O)OC]

NaBr (132 mg, 1.28 mmol) and water (130 μL, 7.34 mmol) were added to a solution of 6-amino-N-(4,4-difluorocyclohexyl)-1,2,4-triazine-5-carboxamide (472 mg, 1.83 mmol) and methyl 2-chloro-4-methoxy-3-oxo-butanoate (663 mg, 3.67 mmol) in DMF (6 mL), and the reaction was heated at 100° C. for 4 h. An additional portion of methyl 2-chloro-4-methoxy-3-oxo-butanoate (331 mg, 1.83 mmol) was added, and the reaction was heated at 100° C. for a further 1 h. The reaction mixture was cooled to room temperature, quenched with water, and extracted with EtOAc (×3). The combined organic layers were washed with brine, dried through a hydrophobic frit, and concentrated in vacuo. The crude product was purified twice by column chromatography (eluting first with 20 to 100% EtOAc in DCM and then with 0 to 100% EtOAc in heptane) to provide the title compound (43 mg) as the impure product. The product was taken forward to the next step without further purification.

¹H NMR (400 MHz, CDCl₃) δ 9.60 (d, J=7.6 Hz, 1H), 9.22 (s, 1H), 4.99 (s, 2H), 4.36-4.26 (m, 1H), 4.08 (s, 3H), 3.55 (s, 3H), 2.23-1.80 (m, 8H)

LCMS (Method 6): 1.66 min, 384.2 [M+H]⁺

### Synthesis of Compound 212, N4-(4,4-difluorocyclohexyl)-6-(methoxymethyl)imidazo[2,1-f][1,2,4]triazine-4,7-dicarboxamide

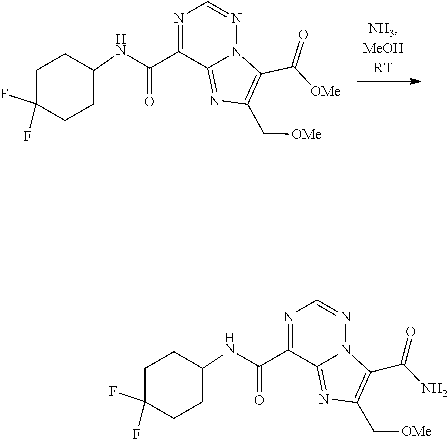 [SMILES: COCc1nc2c(C(=O)NC3CCC(F)(F)CC3)ncnn2c1C(=O)OC ; COCc1nc2c(C(=O)NC3CCC(F)(F)CC3)ncnn2c1C(N)=O]

Methyl 4-[(4,4-difluorocyclohexyl) carbamoyl]-6-(methoxymethyl)imidazo[2,1-f][1,2,4]triazine-7-carboxylate (42 mg, 0.11 mmol) was dissolved in NH₃ (7 M in MeOH) (1.57 mL, 10.96 mmol), and the reaction was stirred at room temperature for 22 h then concentrated in vacuo. The crude product was purified by column chromatography (eluting with 0 to 10% MeOH in DCM) then triturated with heptane to provide the title compound (10 mg).

¹H NMR (400 MHz, DMSO-d₆) δ 9.26 (d, J=6.2 Hz, 2H), 8.25 (s, 1H), 8.09 (s, 1H), 4.92 (s, 2H), 4.17-4.05 (m, 1H), 3.38 (s, 3H), 2.14-1.90 (m, 6H), 1.76-1.64 (m, 2H)

LCMS (Method 7): 1.83 min, 369.2 [M+H]⁺

### Synthesis of Compound 213, N7-(3,3-difluorocyclobutyl)-2-(1-hydroxy-1-methyl-ethyl)pyrazolo[1,5-a]pyrimidine-3,7-dicarboxamide (General Method T)

### Synthesis of 3-amino-5-(1-hydroxy-1-methyl-ethyl)-1H-pyrazole-4-carbonitrile

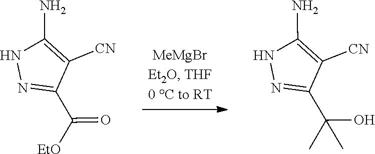

MeMgBr (methyl magnesium bromide; 3 M in Et₂O) (15 mL, 45 mmol) was added dropwise to a solution of ethyl 3-amino-4-cyano-1H-pyrazole-5-carboxylate (1.5 g, 8.33 mmol) in THF (25 mL) at 0° C. The reaction was warmed to room temperature and stirred for 17 h, then the mixture was poured into sat. aq. NH₄Cl. The layers were separated, and the aqueous layer was extracted with EtOAc (×3). The combined organic layers were washed with brine (×2) and the resulting emulsion was filtered through a short pad of Dicalite®. The layers were separated, and the organic layer was dried through a hydrophobic frit and concentrated in vacuo to provide the title compound (733 mg). The crude product was taken forward to the next step without purification.

LCMS (Method 7): 1.08 min, 165.2 [M−H]⁻

### Synthesis of ethyl 3-cyano-2-(1-hydroxy-1-methyl-ethyl)pyrazolo[1,5-a]pyrimidine-7-carboxylate

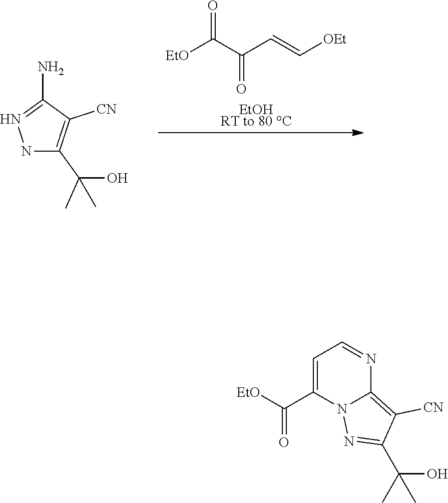

3-Amino-5-(1-hydroxy-1-methyl-ethyl)-1H-pyrazole-4-carbonitrile (733 mg, 4.41 mmol) was added to a solution of ethyl (E)-4-ethoxy-2-oxo-but-3-enoate (1.5 g, 8.71 mmol) in EtOH (15 mL) and the reaction was heated at 80° C. for 4 h. The reaction mixture was cooled to room temperature and concentrated in vacuo. The crude product was purified by column chromatography (eluting with 0 to 35% EtOAc in DCM) to provide the title compound (498 mg).

¹H NMR (400 MHz, DMSO-d₆) δ 8.92 (d, J=4.3 Hz, 1H), 7.70 (d, J=4.3 Hz, 1H), 4.49 (q, J=7.1 Hz, 2H), 1.58 (s, 6H), 1.37 (t, J=7.1 Hz, 3H). Exchangeable proton not observed.

LCMS (Method 7): 2.04 min, 275.2 [M+H]⁺

### Synthesis of 3-cyano-2-(1-hydroxy-1-methyl-ethyl)pyrazolo[1,5-a]pyrimidine-7-carboxylic Acid

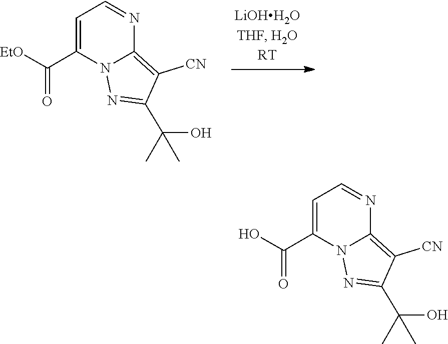 [SMILES: CCOC(=O)c1ccnc2c(C#N)c(C(C)(C)O)nn12 ; CC(C)(O)c1nn2c(C(=O)O)ccnc2c1C#N]

LiOH·H₂O (92 mg, 2.20 mmol) was added to a solution of ethyl 3-cyano-2-(1-hydroxy-1-methyl-ethyl)pyrazolo[1,5-a]pyrimidine-7-carboxylate (498 mg, 1.81 mmol) in THF (4 mL) and water (1 mL), and the reaction was stirred at room temperature for 3 h. The reaction mixture was acidified with 1 M aq. HCl, and the layers were separated. The aqueous layer was extracted with DCM (×2), and the combined organic layers were concentrated in vacuo to provide the title compound (280 mg). The crude product was taken forward to the next step without purification.

¹H NMR (400 MHz, DMSO-d₆): δ 8.88 (d, J=4.3 Hz, 1H), 7.59 (d, J=4.3 Hz, 1H), 1.58 (s, 6H). Exchangeable protons not observed.

LCMS (Method 7): 1.26 min, 247.2 [M+H]⁺

### Synthesis of 3-cyano-N-(3,3-difluorocyclobutyl)-2-(1-hydroxy-1-methyl-ethyl)pyrazolo[1,5-a]pyrimidine-7-carboxamide

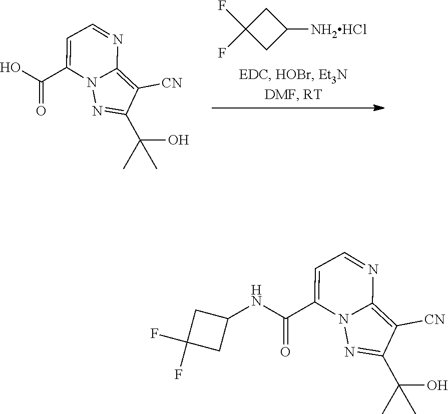

3,3-Difluorocyclobutanamine hydrochloride (294 mg, 2.05 mmol), HOBt (461 mg, 3.41 mmol), EDC (654 mg, 3.41 mmol), and Et₃N (950 μL, 6.82 mmol) were added to a solution of 3-cyano-2-(1-hydroxy-1-methyl-ethyl)pyrazolo[1,5-a]pyrimidine-7-carboxylic acid (280 mg, 1.14 mmol) in DMF (3.5 mL), and the reaction was stirred at room temperature for 3 h.

The reaction mixture was diluted with water and EtOAc and the layers were separated. The aqueous layer was extracted with EtOAc (×2), and the combined organic layers were dried through a hydrophobic frit and concentrated in vacuo. The crude product was purified by column chromatography (eluting with 0 to 100% EtOAc in heptane) to provide the title compound (287 mg).

¹H NMR (400 MHz, DMSO-d₆): δ 9.97 (d, J=6.6 Hz, 1H), 8.95 (d, J=4.3 Hz, 1H), 7.73-7.68 (m, 1H), 4.43-4.31 (m, 1H), 3.29 (s, 1H), 3.20-3.04 (m, 2H), 2.79-2.65 (m, 2H), 1.62 (s, 6H).

LCMS (Method 7): 2.23 min, 336.2 [M+H]⁺

### Synthesis of Compound 213, N7-(3,3-difluorocyclobutyl)-2-(1-hydroxy-1-methyl-ethyl)pyrazolo[1,5-a]pyrimidine-3,7-dicarboxamide

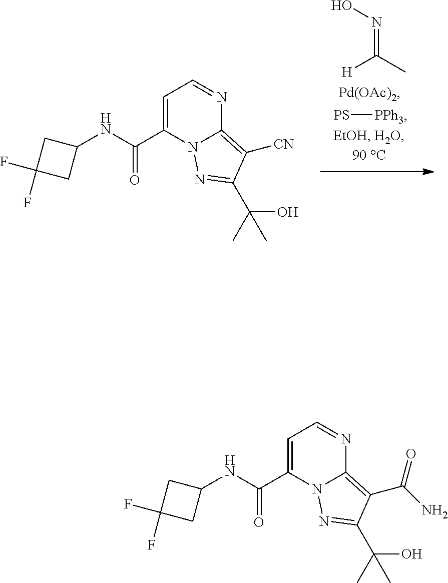

3-Cyano-N-(3,3-difluorocyclobutyl)-2-(1-hydroxy-1-methyl-ethyl)pyrazolo[1,5-a]pyrimidine-7-carboxamide (287 mg, 0.86 mmol), acetaldehyde oxime (200 mg, 3.39 mmol), Pd(OAc)₂ (20 mg, 0.09 mmol), and PS-PPh₃ (1.4-2 mmol/g) (175 mg, 0.25-0.35 mmol) were suspended in EtOH (2 mL) and water (400 μL) and the reaction was heated at 90° C. for 3 h. The reaction mixture was cooled to room temperature and filtered through a short pad of

Dicalite®. The Dicalite® was slurried with a 1:1 mixture of MeOH:DCM, filtered, and washed with a further 1:1 mixture of MeOH:DCM. The filtrate was concentrated in vacuo, and the crude product was purified by preparative HPLC (acidic method) to provide the title compound (66 mg).

¹H NMR (400 MHz, DMSO-d₆) δ 10.16 (d, J=6.6 Hz, 1H), 8.96 (d, J=4.4 Hz, 1H), 8.31 (d, J=2.7 Hz, 1H), 8.14 (d, J=2.4 Hz, 1H), 7.71 (d, J=4.4 Hz, 1H), 7.56 (s, 1H), 4.45-4.31 (m, 1H), 3.22-3.07 (m, 2H), 2.80-2.62 (m, 2H), 1.60 (s, 6H)

LCMS (Method 7): 2.23 min, 354.2 [M+H]⁺

### Synthesis of Compound 214, N7-indan-2-yl-2-(methylsulfanylmethyl)pyrazolo[1,5-a]pyrimidine-3,7-dicarboxamide (General Method U)

### Synthesis of [3-carbamoyl-7-(indan-2-ylcarbamoyl)pyrazolo[1,5-a]pyrimidin-2-yl]methyl methanesulfonate

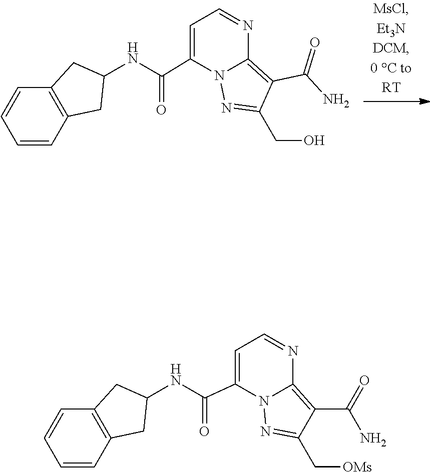

MsCl (methanesulfonyl chloride; 10 μL, 0.13 mmol) was added dropwise to a solution of 2-(hydroxymethyl)-N7-indan-2-yl-pyrazolo[1,5-a]pyrimidine-3,7-dicarboxamide (41 mg, 0.11 mmol) and Et₃N (24 μL, 0.17 mmol) in DCM (3 mL) at 0° C. The reaction was stirred at 0° C. for 10 min, then warmed to room temperature and stirred for 18 h. The reaction mixture was cooled to 0° C. and additional portions of Et₃N (48 μL, 0.34 mmol) and MsCl (20 μL, 0.25 mmol) were added. The reaction was stirred at 0° C. for 10 min, then warmed to room temperature and stirred for a further 4 h. The reaction mixture was diluted with DCM and washed with sat. aq. Na₂CO₃. The aqueous layer was extracted with DCM, and the combined organic layers were washed with brine, dried over Na₂SO₄, and concentrated in vacuo to provide the title compound (49 mg). The crude product was taken forward to the next step without purification.

¹H NMR (400 MHz, CDCl₃) δ 10.42 (d, J=7.0 Hz, 1H), 8.84 (d, J=4.4 Hz, 1H), 8.02 (d, J=4.4 Hz, 1H), 7.99 (s, 1H), 7.30-7.27 (m, 2H), 7.22-7.16 (m, 2H), 5.85 (s, 2H), 5.64 (s, 1H), 5.00-4.94 (m, 1H), 3.48 (d, J=7.4 Hz, 1H), 3.44 (d, J=7.4 Hz, 1H), 3.18 (d, J=6.2 Hz, 1H), 3.14 (d, J=6.1 Hz, 1H), 3.08 (s, 3H)

LCMS (Method 6): 1.12 min, 430.2 [M+H]⁺

### Synthesis of Compound 214, N7-indan-2-yl-2-(methylsulfanylmethyl)pyrazolo[1,5-a]pyrimidine-3,7-dicarboxamide

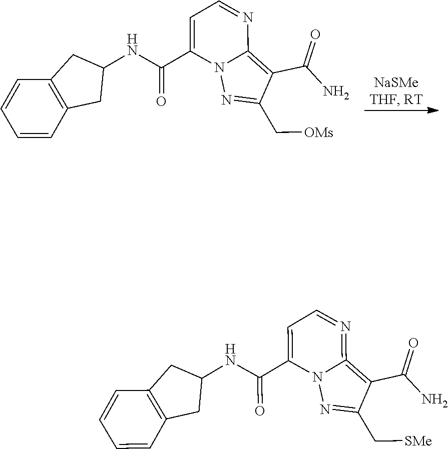

NaSMe (sodium methanthiolate; 21 wt % in water; 57 μL, 0.17 mmol) was added to a solution of [3-carbamoyl-7-(indan-2-ylcarbamoyl)pyrazolo[1,5-a]pyrimidin-2-yl]methyl methanesulfonate (49 mg, 0.11 mmol) in THF (1 mL) and the reaction was stirred at room temperature for 1 h. An additional portion of NaSMe (21 wt % in water; 19 μL, 0.06 mmol) was added, and the reaction was stirred for a further 18 h. The reaction mixture was diluted with EtOAc and washed with brine. The aqueous layer was extracted with EtOAc and DCM, and the combined organic layers were dried over Na₂SO₄ and concentrated in vacuo. The crude product was purified by column chromatography (eluting with 0 to 2% MeOH in DCM) to provide the title compound (16 mg).

¹H NMR (400 MHz, DMSO-d₆) δ 10.17 (d, J=7.2 Hz, 1H), 8.90 (d, J=4.4 Hz, 1H), 7.68 (d, J=4.3 Hz, 2H), 7.57 (s, 1H), 7.31-7.27 (m, 2H), 7.20-7.16 (m, 2H), 4.83-4.77 (m, 1H), 4.13 (s, 2H), 3.40 (d, J=7.2 Hz, 1H), 3.36 (d, J=7.2 Hz, 1H), 2.99 (d, J=4.6 Hz, 1H), 2.95 (d, J=4.5 Hz, 1H), 1.97 (s, 3H)

LCMS (Method 7): 2.61 min, 382.2 [M+H]⁺

### Synthesis of Compound 217, 2-(methylsulfanylmethyl)-N7-[cis-3-(trifluoromethoxy)cyclobutyl]pyrazolo[1,5-a]pyrimidine-3,7-dicarboxamide

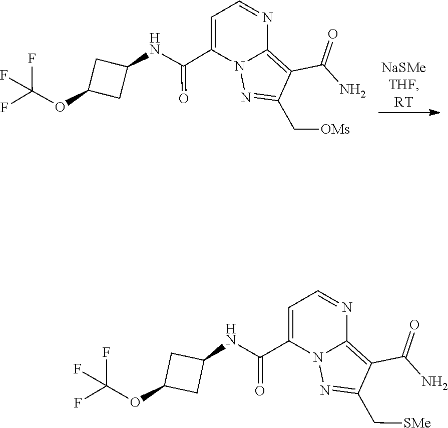 [SMILES: CS(=O)(=O)OCc1nn2c(C(=O)N[C@H]3C[C@@H](OC(F)(F)F)C3)ccnc2c1C(N)=O ; CSCc1nn2c(C(=O)N[C@H]3C[C@@H](OC(F)(F)F)C3)ccnc2c1C(N)=O]

2-(Methylsulfanylmethyl)-N7-[cis-3-(trifluoromethoxy)cyclobutyl]pyrazolo[1,5-a]pyrimidine-3,7-dicarboxamide (14 mg), was synthesised following a similar procedure to that described in General Method U.

¹H NMR (400 MHz, CDCl₃) δ 10.69 (d, J=7.0 Hz, 1H), 8.79 (d, J=4.4 Hz, 1H), 8.06 (br. s, 1H), 7.91 (d, J=4.4 Hz, 1H), 5.62 (br. s, 1H), 4.57-4.50 (m, 1H), 4.44 (s, 2H), 4.34-4.26 (m, 1H), 3.08-3.00 (m, 2H), 2.44-2.35 (m, 2H), 2.27 (s, 3H)

LCMS (Method 7): 2.68 min, 404.1 [M+H]⁺

### Synthesis of Compound 215, N7-indan-2-yl-2-(methylsulfonylmethyl)pyrazolo[1,5-a]pyrimidine-3,7-dicarboxamide

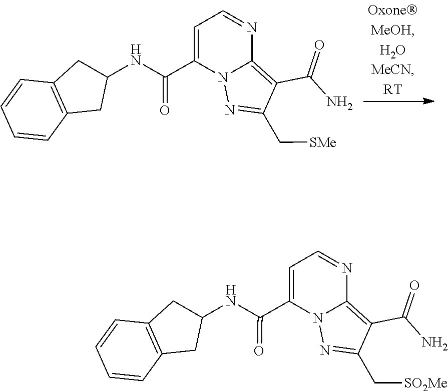 [SMILES: CSCc1nn2c(C(=O)NC3Cc4ccccc4C3)ccnc2c1C(N)=O ; CS(=O)(=O)Cc1nn2c(C(=O)NC3Cc4ccccc4C3)ccnc2c1C(N)=O]

N7-indan-2-yl-2-(methylsulfanylmethyl)pyrazolo[1,5-a]pyrimidine-3,7-dicarboxamide (8 mg, 0.02 mmol) and Oxone® (19 mg, 0.03 mmol) were dissolved in MeOH (2 mL), and the reaction was stirred at room temperature for 19 h. An additional portion of Oxone® (19 mg, 0.03 mmol) was added, and the reaction was stirred at room temperature for a further 20 h. A third portion of Oxone® (60 mg, 0.10 mmol) was added, and the reaction was stirred at room temperature for 4 days. The reaction mixture was concentrated in vacuo, and the residue was suspended in MeCN (3 mL) and water (3 mL). A final portion of Oxone® (19 mg, 0.03 mmol) was added, and the reaction was stirred at room temperature for 16 h. The reaction mixture was concentrated in vacuo, and the residue was dissolved in EtOAc and water. The layers were separated, and the organic layer was dried through a hydrophobic frit and concentrated in vacuo. The residue was dissolved in DCM and water, and the layers were separated. The organic layer was dried through a hydrophobic frit and concentrated in vacuo. The crude product was dissolved in a 2:1 mixture of EtOAc:DCM and filtered through a short plug of silica (eluting with EtOAc). The filtrate was concentrated in vacuo, and the resulting solid was washed with heptane and dried to provide the title compound (3.8 mg).

¹H NMR (400 MHz, CDCl₃) δ 10.46 (d, J=7.1 Hz, 1H), 8.86 (d, J=4.4 Hz, 1H), 8.08 (s, 1H), 8.05 (d, J=4.4 Hz, 1H), 7.32-7.27 (m, 2H), 7.25-7.19 (m, 2H), 5.66 (s, 1H), 5.05 (s, 2H), 5.01-4.89 (m, 1H), 3.48 (dd, J=16.0, 7.1 Hz, 2H), 3.15-3.05 (m, 2H), 2.88 (s, 3H)

LCMS (Method 4): 1.99 min, 414.0 [M+H]⁺

### Synthesis of Compound 216, 2-(cyclopropoxymethyl)-N7-[cis-3-(trifluoromethoxy)cyclobutyl]pyrazolo[1,5-a]pyrimidine-3,7-dicarboxamide (General Method V)

### Synthesis of 3-bromo-2-(hydroxymethyl)-N-[3-(cis-trifluoromethoxy)cyclobutyl]pyrazolo[1,5-a]pyrimidine-7-carboxamide

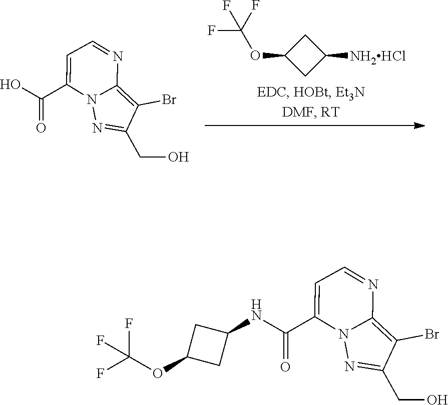

3-(Cis-trifluoromethoxy)cyclobutanamine hydrochloride (634 mg, 3.31 mmol), HOBt (324 mg, 2.40 mmol), EDC (1.27 g, 6.62 mmol), and Et₃N (1.84 mL, 13.23 mmol) were added to a suspension of 3-bromo-2-(hydroxymethyl)pyrazolo[1,5-a]pyrimidine-7-carboxylic acid (600 mg, 2.21 mmol) in DMF (6.5 mL) and the reaction was stirred at room temperature for 18 h. The reaction mixture was diluted with DCM and water and the layers were separated. The organic layer was washed with water (×3) and brine, dried over Na₂SO₄, and concentrated in vacuo. The crude product was purified by column chromatography (eluting with 0 to 100% EtOAc in heptane) to provide the title compound (553 mg).

¹H NMR (400 MHz, CDCl₃) δ 10.34 (d, J=6.7 Hz, 1H), 8.68 (d, J=4.2 Hz, 1H), 7.77 (d, J=4.2 Hz, 1H), 4.98-4.92 (m, 2H), 4.53-4.41 (m, 1H), 4.31-4.17 (m, 1H), 3.02-2.91 (m, 2H), 2.37-2.28 (m, 2H), 2.02 (s, 1H)

LCMS (Method 7): 2.58 min, 409.0, 411.0 [M+H]⁺

### Synthesis of 3-bromo-2-(bromomethyl)-N-[cis-3-(trifluoromethoxy)cyclobutyl]pyrazolo[1,5-a]pyrimidine-7-carboxamide

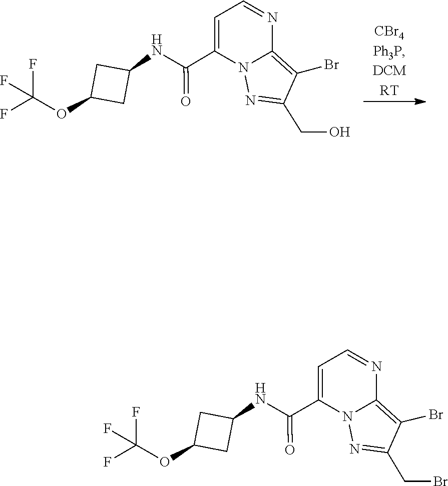 [SMILES: O=C(N[C@H]1C[C@@H](OC(F)(F)F)C1)c1ccnc2c(Br)c(CO)nn12 ; O=C(N[C@H]1C[C@@H](OC(F)(F)F)C1)c1ccnc2c(Br)c(CBr)nn12]

CBr₄ (665 mg, 2 mmol) and Ph₃P (526 mg, 2 mmol) were added to a solution of 3-bromo-2-(hydroxymethyl)-N-[cis-3-(trifluoromethoxy)cyclobutyl]pyrazolo[1,5-a]pyrimidine-7-carboxamide (410 mg, 1 mmol) in DCM (13 mL), and the reaction was stirred at room temperature for 18 h. The reaction mixture was concentrated in vacuo, and the crude product was purified by column chromatography (eluting with 0 to 100% EtOAc in heptane) to provide the title compound (476 mg).

¹H NMR (400 MHz, CDCl₃) δ 10.31 (d, J=7.0 Hz, 1H), 8.76 (d, J=4.2 Hz, 1H), 7.86 (d, J=4.2 Hz, 1H), 4.72 (s, 2H), 4.54 (p, J=7.3 Hz, 1H), 4.38-4.24 (m, 1H), 3.10-2.98 (m, 2H), 2.47-2.35 (m, 2H)

LCMS (Method 7): 3.27 min, 471.0, 473.0, 475.0 [M+H]⁺

### Synthesis of 3-bromo-2-(cyclopropoxymethyl)-N-[cis-3-(trifluoromethoxy)cyclobutyl]pyrazolo[1,5-a]pyrimidine-7-carboxamide

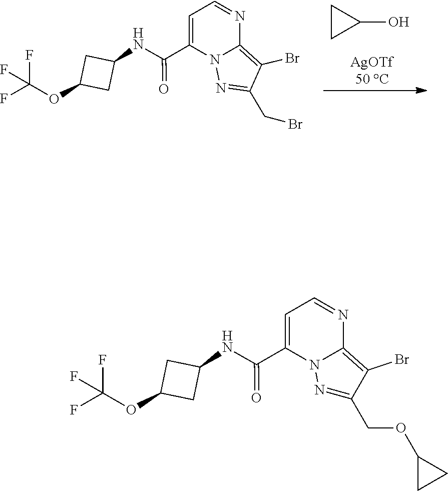

AgOTf (131 mg, 0.51 mmol) was added to a suspension of 3-bromo-2-(bromomethyl)-N-[cis-3-(trifluoromethoxy)cyclobutyl]pyrazolo[1,5-a]pyrimidine-7-carboxamide (160 mg, 0.34 mmol) in cyclopropanol (1.7 mL) and the reaction was heated at 50° C. for 2 h. The reaction mixture was cooled to room temperature and diluted with EtOAc and water. The layers were separated, and the organic layer was dried through a hydrophobic frit and concentrated in vacuo. The crude product was purified by column chromatography (eluting with 0 to 100% EtOAc in heptane) to provide the title compound (79 mg).

¹H NMR (400 MHz, CDCl₃) δ 10.51 (d, J=7.1 Hz, 1H), 8.74 (d, J=4.2 Hz, 1H), 7.82 (d, J=4.2 Hz, 1H), 4.86 (s, 2H), 4.60-4.49 (m, 1H), 4.36-4.24 (m, 1H), 3.59-3.52 (m, 1H), 3.08-2.97 (m, 2H), 2.46-2.33 (m, 2H), 0.79-0.73 (m, 2H), 0.62-0.55 (m, 2H)

LCMS (Method 7): 3.28 min, 449.0, 451.0 [M+H]⁺

### Synthesis of Compound 216, 2-(cyclopropoxymethyl)-N7-[cis-3-(trifluoromethoxy)cyclobutyl]pyrazolo[1,5-a]pyrimidine-3,7-dicarboxamide

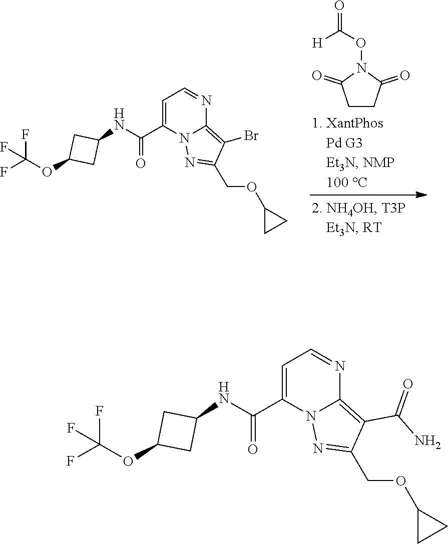 [SMILES: O=C(N[C@H]1C[C@@H](OC(F)(F)F)C1)c1ccnc2c(Br)c(COC3CC3)nn12 ; O=CON1C(=O)CCC1=O ; NC(=O)c1c(COC2CC2)nn2c(C(=O)N[C@H]3C[C@@H](OC(F)(F)F)C3)ccnc12]

3-Bromo-2-(cyclopropoxymethyl)-N-[cis-3-(trifluoromethoxy)cyclobutyl]pyrazolo[1,5-a]pyrimidine-7-carboxamide (79 mg, 0.18 mmol), Et₃N (74 μL, 0.53 mmol), and NMP (1.6 mL) were placed in a vial, and the vial was sealed and purged with argon for 5 min. XantPhos Pd G3 (17 mg, 0.02 mmol) was added, and the vial was purged with argon for a further 5 min. (2,5-Dioxopyrrolidin-1-yl) formate (126 mg, 0.88 mmol) was added and the vial was sealed immediately. The reaction was heated at 100° C. in a microwave reactor for 2 h. Additional portions of Et₃N (74 μL, 0.53 mmol) and XantPhos Pd G3 (17 mg, 0.02 mmol) were added, and the reaction mixture was degassed with argon for 5 min. Additional (2,5-dioxopyrrolidin-1-yl) formate (126 mg, 0.88 mmol) was added, and the reaction was heated at 100° C. in a microwave reactor for a further 2 h. The reaction mixture was cooled to room temperature, and Et₃N (74 μL, 0.53 mmol), 1-propanephosphonic anhydride (50 wt % in DMF) (209 μL, 0.35 mmol), and NH₄OH (230 μL, 1.76 mmol) were added. The reaction was stirred at room temperature for 18 h. The reaction mixture was diluted with EtOAc and water, and the layers were separated. The aqueous layer was extracted with EtOAc (×3), and the combined organic layers were dried through a hydrophobic frit and concentrated in vacuo. The crude product was purified by preparative HPLC (acidic method) to provide the title compound (14 mg).

¹H NMR (400 MHz, DMSO-d₆) δ 10.17 (d, J=7.5 Hz, 1H), 8.91 (d, J=4.3 Hz, 1H), 7.75-7.56 (m, 3H), 5.08 (s, 2H), 4.72 (p, J=7.2 Hz, 1H), 4.29-4.14 (m, 1H), 3.67-3.58 (m, 1H), 2.97-2.86 (m, 2H), 2.33-2.23 (m, 2H), 0.66-0.57 (m, 2H), 0.53-0.43 (m, 2H)

LCMS (Method 7): 2.65 min, 414.2 [M+H]⁺

### Example 2—GPR68 Biochemical Assay

GPR68 antagonism was determined using the Promega GloSensor™ CAMP assay (F. I. Wang et al., Methods 2021, S1046-2023 (21) 00249-8). HEK293T cells (obtained from ECACC, Cat 12022001, Lot no. 17E044) were cultured in T175 flasks (Greiner, Cat 660175) in DMEM (Gibco, Cat 11995-065) supplemented with 10% Fetal Bovine Serum (Gibco Cat 16000-044), and 100 u/mL Pen-Strep (Gibco, Cat 15140) at 37° C. and 5% CO₂. GPR68 expression was achieved by transient transfection with a mammalian expression plasmid encoding full-length human GPR68 (uniprot ID: Q15743) under control of a CMV promoter. The day before transfection, cell media was aspirated and HEK293T cells were incubated with accutase (Sigma, Cat A6964) for 3-4 minutes 37° C. and 5% CO₂ before being manually detached with excess DMEM and trituration. Cell density was determined by automated cell counting (Countess, Invitrogen) and adjusted to 6×10⁶ cells/20 mL and transferred to 15 cm dishes (Thermo Fisher, Cat 130183) for overnight incubation at 37° C. and 5% CO₂. The next day separate mammalian expression plasmids encoding hGPR68 (4 μg) and Promega GloSensor (8 μg) were diluted to 1 mL in Opti-MEM (Thermo Fisher Scientific, Cat 31985070) and incubated with 30 μL of lipofectamine 3000 (also diluted to 1 mL in Opti-MEM) for 15 minutes at room temperature and subsequently added dropwise to the plated cells. Post transfection cells were then incubated at 37° C. and 5% CO₂ for at least 16 hours before transfer to assay plates the following day. Transfer was conducted by washing cells with PBS and incubating with accutase for 3-4 minutes 37° C. and 5% CO₂ before being manually detached with excess DMEM and mild trituration. The suspension was then centrifuged at 192 RCF for 4 minutes in a 50 mL centrifuge tube (Greiner, Cat 227261).

Supernatant was removed by aspiration and the cell pellet was resuspended in DMEM+1% dialysed FBS (Gibco, Cat 26400-044) and 100 u/mL Pen-Strep to a cell density of 500 cells/μL. Cells were plated into poly-L-Lysine coated 384-well white assay plates (Corning, Cat 3570) at a final volume of 40 UL and a density of 20,000 cells/well before overnight incubation at 37° C. and 5% CO₂. The following day media was manually ejected from the 384-well assay plate and replaced with 10 μL GloSensor substrate (Promega, Cat E1291) prepared in pH 8.0 assay buffer (1×HBSS, 10 mM HEPES, 10 mM MOPS). Cells were incubated with this solution for 60 minutes at room temperature in the dark. Test compounds or an equivalent volume of DMSO (vehicle) were added to compounds plates to create a 9-point, half-log concentration series for each compound with a maximum final assay concentration of 50 μM and a consistent final DMSO concentration of 0.5%. Assay buffer with 0.1% BSA and 0.01% ascorbic acid was prepared at three pHs: pH 8.0, pH 5.5 and pH 4.5. pH 8.0 and 4.5 were added to the compound plate as maximum and minimum effect controls whilst pH 5.5 was added to all compound wells to stimulate GPR68 to a level corresponding to 90% of maximal stimulation after addition to the cells already in pH 8.0 (assay acceptance criteria range 70-90%). DMSO/Compound and buffer was added to the plated cells containing the GloSensor substrate and incubated for 15 minutes at room temperature before being read on a microplate reader (BMG PHERAstar, optic module LUM Plus with Gain 3600). Data was exported and analysed in excel and Graphpad prism (version 9). Assay QC acceptance criteria required the maximum and minimum controls to return a Z′≥0.5, the EC₈₀ to be 80%±10% of expected and the reference ligand pIC₅₀ to be within one-half-log unit of its historical IC₅₀. Percent inhibition was calculated using the mean EC₈₀ (pH 5.5) and mean minimum effect control (pH 8.0). Data were plotted as percentage inhibition, average±SEM. IC₅₀, Emax, Emin, Actual max, actual min, R² and fit confidence were calculated using Graphpad Prism's sigmoidal dose-response function.

TABLE 2
hGPR68 biochemical IC₅₀ data

| Compound | hGPR68 | |
|---|---|---|
| number | IC₅₀ | Structure name |
| 1 | D | N7-indan-2-ylpyrazolo[1,5-a]pyrimidine-3,7-dicarboxamide |
| 2 | D | N7-(4-methylindan-2-yl)pyrazolo[1,5-a]pyrimidine-3,7- |
| | | dicarboxamide |
| 3 | A | N7-(5-methoxyindan-2-yl)pyrazolo[1,5-a]pyrimidine-3,7- |
| | | dicarboxamide |
| 4 | D | N7-(4-chloroindan-2-yl)pyrazolo[1,5-a]pyrimidine-3,7- |
| | | dicarboxamide |
| 5 | C | N7-(1,2,3,4-tetrahydroquinolin-3-yl)pyrazolo[1,5- |
| | | a]pyrimidine-3,7-dicarboxamide |
| 6 | D | N7-chroman-3-ylpyrazolo[1,5-a]pyrimidine-3,7- |
| | | dicarboxamide |
| 7 | B | N7-(trans-3-fluoro-3-methyl-cyclobutyl)pyrazolo[1,5- |
| | | a]pyrimidine-3,7-dicarboxamide |
| 8 | B | N7-(cis-3-hydroxy-3-methyl-cyclobutyl)pyrazolo[1,5- |
| | | a]pyrimidine-3,7-dicarboxamide |
| 9 | C | N7-(cis-3-cyanocyclobutyl)pyrazolo[1,5-a]pyrimidine-3,7- |
| | | dicarboxamide |
| 10 | A | N7-(3,3-diethylcyclobutyl)pyrazolo[1,5-a]pyrimidine-3,7- |
| | | dicarboxamide |
| 11 | B | N7-(cis-3-methoxycyclobutyl)pyrazolo[1,5-a]pyrimidine-3,7- |
| | | dicarboxamide |
| 12 | A | N7-(7-oxaspiro[3.5]nonan-2-yl)pyrazolo[1,5-a]pyrimidine- |
| | | 3,7-dicarboxamide |
| 13 | A | N7-(5,6,7,8-tetrahydroisoquinolin-7-yl)pyrazolo[1,5- |
| | | a]pyrimidine-3,7-dicarboxamide |
| 14 | C | N7-[4-(3,3,3-trifluoropropyl)indan-2-yl]pyrazolo[1,5- |
| | | a]pyrimidine-3,7-dicarboxamide |
| 15 | B | N7-[5-(2-pyrrolidin-1-ylethyl)indan-2-yl]pyrazolo[1,5- |
| | | a]pyrimidine-3,7-dicarboxamide |
| 16 | A | N7-[4-(4-chlorophenoxy)indan-2-yl]pyrazolo[1,5- |
| | | a]pyrimidine-3,7-dicarboxamide |
| 17 | A | N7-[4-(2-pyridyl)indan-2-yl]pyrazolo[1,5-a]pyrimidine-3,7- |
| | | dicarboxamide |
| 18 | C | N7-[cis-3-(trifluoromethoxy)cyclobutyl]pyrazolo[1,5- |
| | | a]pyrimidine-3,7-dicarboxamide |
| 19 | B | N7-(6,7-dihydro-5H-pyrrolo[1,2-a]imidazol-6- |
| | | yl)pyrazolo[1,5-a]pyrimidine-3,7-dicarboxamide |
| 20 | B | N7-(1,2,3,4-tetrahydro-1,8-naphthyridin-3-yl)pyrazolo[1,5- |
| | | a]pyrimidine-3,7-dicarboxamide |
| 21 | D | 2-cyano-N7-indan-2-yl-pyrazolo[1,5-a]pyrimidine-3,7- |
| | | dicarboxamide |
| 22 | C | N7-indan-2-yl-2-pyridazin-3-yl-pyrazolo[1,5-a]pyrimidine- |
| | | 3,7-dicarboxamide |
| 23 | A | 2-[2-(dimethylamino)ethylamino]-N7-indan-2-yl- |
| | | pyrazolo[1,5-a]pyrimidine-3,7-dicarboxamide |
| 24 | A | N7-indan-2-yl-2-(1-methylazetidin-3-yl)pyrazolo[1,5- |
| | | a]pyrimidine-3,7-dicarboxamide |
| 25 | C | N7-(3,3-difluorocyclobutyl)-2-oxazol-2-yl-pyrazolo[1,5- |
| | | a]pyrimidine-3,7-dicarboxamide |
| 26 | D | N8-(3,3-difluorocyclobutyl)-2-methyl-imidazo[1,2- |
| | | blpyridazine-3,8-dicarboxamide |
| 27 | E | N7-indan-2-yl-2-(isopropoxymethyl)pyrazolo[1,5- |
| | | a]pyrimidine-3,7-dicarboxamide |
| 28 | B | N7-(3,3-difluorocyclobutyl)-2- |
| | | (isopropoxymethyl)pyrazolo[1,5-a]pyrimidine-3,7- |
| | | dicarboxamide |
| 29 | C | 2-[(dimethylamino)methyl]-N7-indan-2-yl-pyrazolo[1,5- |
| | | a]pyrimidine-3,7-dicarboxamide |
| 30 | C | N7-indan-2-yl-2-(methylaminomethyl)pyrazolo[1,5- |
| | | a]pyrimidine-3,7-dicarboxamide |
| 31 | B | N-indan-2-yl-3-oxazol-2-yl-pyrazolo[1,5-a]pyridine-7- |
| | | carboxamide |
| 32 | C | N7-(3,3-difluorocyclobutyl)-2-methyl-6-(1H-pyrazol-4- |
| | | yl)pyrazolo[1,5-a]pyrimidine-3,7-dicarboxamide |
| 33 | E | N7-indan-2-yl-2-oxazol-2-yl-pyrazolo[1,5-a]pyrimidine-3,7- |
| | | dicarboxamide |
| 34 | E | N7-(3,3-difluorocyclobutyl)-2-(methylamino)pyrazolo[1,5- |
| | | a]pyrimidine-3,7-dicarboxamide |
| 35 | C | N7-(3,3-difluorocyclobutyl)-2-(dimethylamino)pyrazolo[1,5- |
| | | a]pyrimidine-3,7-dicarboxamide |
| 36 | C | 2-(2-fluorophenyl)-N7-indan-2-yl-pyrazolo[1,5-a]pyridine- |
| | | 3,7-dicarboxamide |
| 37 | E | N7-indan-2-yl-2-tetrahydrofuran-3-yl-pyrazolo[1,5- |
| | | a]pyrimidine-3,7-dicarboxamide |
| 38 | B | N7-(3,3-difluorocyclobutyl)-2,6-dimethyl-pyrazolo[1,5- |
| | | a]pyrimidine-3,7-dicarboxamide |
| 39 | C | N7-(3,3-difluorocyclobutyl)-5-(methoxymethyl)-2-methyl- |
| | | pyrazolo[1,5-a]pyrimidine-3,7-dicarboxamide |
| 40 | A | N7-indan-2-yl-N3-methyl-2-phenyl-pyrazolo[1,5-α]pyridine- |
| | | 3,7-dicarboxamide |
| 41 | C | N7-(3,3-difluorocyclobutyl)-5-methoxy-2-methyl- |
| | | pyrazolo[1,5-a]pyrimidine-3,7-dicarboxamide |
| 42 | D | N7-(3,3-difluorocyclobutyl)-2,5-dimethyl-pyrazolo[1,5- |
| | | a]pyrimidine-3,7-dicarboxamide |
| 43 | D | N7-(3,3-difluorocyclobutyl)-2-tetrahydrofuran-2-yl- |
| | | pyrazolo[1,5-a]pyrimidine-3,7-dicarboxamide |
| 44 | D | N7-indan-2-yl-2-tetrahydrofuran-2-yl-pyrazolo[1,5- |
| | | a]pyrimidine-3,7-dicarboxamide |
| 45 | C | N7-(3,3-difluorocyclobutyl)-2-tetrahydrofuran-3-yl- |
| | | pyrazolo[1,5-a]pyrimidine-3,7-dicarboxamide |
| 46 | E | N7-indan-2-yl-2-methyl-pyrazolo[1,5-a]pyrimidine-3,7- |
| | | dicarboxamide |
| 47 | C | N7-chroman-3-yl-2-methyl-pyrazolo[1,5-a]pyrimidine-3,7- |
| | | dicarboxamide |
| 48 | D | N7-(3,3-difluorocyclobutyl)-2-methyl-pyrazolo[1,5- |
| | | a]pyrimidine-3,7-dicarboxamide |
| 49 | C | N7-(6,7-dihydro-5H-cyclopenta[b]pyridin-6-yl)-2-methyl- |
| | | pyrazolo[1,5-a]pyrimidine-3,7-dicarboxamide |
| 50 | C | N7-(cis-3-cyanocyclobutyl)-2-methyl-pyrazolo[1,5- |
| | | a]pyrimidine-3,7-dicarboxamide |
| 51 | B | N7-(5-benzylindan-2-yl)-2-methyl-pyrazolo[1,5- |
| | | a]pyrimidine-3,7-dicarboxamide |
| 52 | B | N7-(6,7-dihydro-5H-cyclopenta[c]pyridin-6-yl)-2-methyl- |
| | | pyrazolo[1,5-a]pyrimidine-3,7-dicarboxamide |
| 53 | D | 2-methyl-N7-[cis-3- |
| | | (trifluoromethoxy)cyclobutyl]pyrazolo[1,5-a]pyrimidine-3,7- |
| | | dicarboxamide |
| 54 | E | N8-(3,3-difluorocyclobutyl)-2-(methoxymethyl)imidazo[1,2- |
| | | b]pyridazine-3,8-dicarboxamide |
| 55 | D | 2-(methoxymethyl)-N8-[cis-3- |
| | | (trifluoromethoxy)cyclobutyl]imidazo[1,2-b]pyridazine-3,8- |
| | | dicarboxamide |
| 56 | E | N8-indan-2-yl-2-(methoxymethyl)imidazo[1,2-b]pyridazine- |
| | | 3,8-dicarboxamide |
| 57 | E | N7-indan-2-yl-2-(methoxymethyl)pyrazolo[1,5-a]pyrimidine- |
| | | 3,7-dicarboxamide |
| 58 | E | N7-(3,3-difluorocyclobutyl)-2-(methoxymethyl)pyrazolo[1,5- |
| | | a]pyrimidine-3,7-dicarboxamide |
| 59 | E | 2-(methoxymethyl)-N7-[5-(trifluoromethyl)-2- |
| | | pyridyl]pyrazolo[1,5-a]pyrimidine-3,7-dicarboxamide |
| 60 | E | N7-(6,7-dihydro-5H-cyclopenta[b]pyridin-6-yl)-2- |
| | | (methoxymethyl)pyrazolo[1,5-a]pyrimidine-3,7- |
| | | dicarboxamide |
| 61 | E | 2-(methoxymethyl)-N7-[cis-3- |
| | | (trifluoromethoxy)cyclobutyl]pyrazolo[1,5-a]pyrimidine-3,7- |
| | | dicarboxamide |
| 62 | C | N7-[4-[(dimethylamino)methyl]phenyl]-2- |
| | | (methoxymethyl)pyrazolo[1,5-a]pyrimidine-3,7- |
| | | dicarboxamide |
| 63 | C | 2-(methoxymethyl)-N7-[(1-methylpyrazol-4- |
| | | yl)methyl]pyrazolo[1,5-a]pyrimidine-3,7-dicarboxamide |
| 64 | E | N7-(4,6-dimethyl-2-pyridyl)-2-(methoxymethyl)pyrazolo[1,5- |
| | | α]pyrimidine-3,7-dicarboxamide |
| 65 | D | 2-(methoxymethyl)-N7-94-(trifluoromethyl)-2- |
| | | pyridyl]pyrazolo[1,5-α]pyrimidine-3,7-dicarboxamide |
| 66 | E | N7-(3,4-difluorophenyl)-2-(methoxymethyl)pyrazolo[1,5- |
| | | α]pyrimidine-3,7-dicarboxamide |
| 67 | D | N7-(2,3-dihydrobenzofuran-5-ylmethyl)-2- |
| | | (methoxymethyl)pyrazolo[1,5-α]pyrimidine-3,7- |
| | | dicarboxamide |
| 68 | E | N7-(2,4-difluorophenyl)-2-(methoxymethyl)pyrazolo[1,5- |
| | | a]pyrimidine-3,7-dicarboxamide |
| 69 | D | N7-benzyl-2-(methoxymethyl)pyrazolo[1,5-α]pyrimidine- |
| | | 3,7-dicarboxamide |
| 70 | E | N7-[(4-fluorophenyl)methyl]-2- |
| | | (methoxymethyl)pyrazolo[1,5-α]pyrimidine-3,7- |
| | | dicarboxamide |
| 71 | D | N7-[(3-fluorophenyl)methyl]-2- |
| | | (methoxymethyl)pyrazolo[1,5-α]pyrimidine-3,7- |
| | | dicarboxamide |
| 72 | E | 2-(methoxymethyl)-N7-(p-tolylmethyl)pyrazolo[1,5- |
| | | a]pyrimidine-3,7-dicarboxamide |
| 73 | E | 2-(methoxymethyl)-N7-(7-methoxytetralin-2- |
| | | yl)pyrazolo[1,5-a]pyrimidine-3,7-dicarboxamide |
| 74 | E | 2-(methoxymethyl)-N7-[(1R)-tetralin-1-yl]pyrazolo[1,5- |
| | | a]pyrimidine-3,7-dicarboxamide |
| 75 | E | N7-(2,3-dihydrobenzofuran-3-yl)-2- |
| | | (methoxymethyl)pyrazolo[1,5-a]pyrimidine-3,7- |
| | | dicarboxamide |
| 76 | D | N7-cyclohexyl-2-(methoxymethyl)pyrazolo[1,5- |
| | | a]pyrimidine-3,7-dicarboxamide |
| 77 | C | N7-[(2,2-dimethylcyclopropyl)methyl]-2- |
| | | (methoxymethyl)pyrazolo[1,5-a]pyrimidine-3,7- |
| | | dicarboxamide |
| 78 | D | N7-chroman-3-yl-2-(methoxymethyl)pyrazolo[1,5- |
| | | a]pyrimidine-3,7-dicarboxamide |
| 79 | E | 2-(methoxymethyl)-N7-[(2S)-tetralin-2-yl]pyrazolo[1,5- |
| | | a]pyrimidine-3,7-dicarboxamide |
| 80 | E | N7-(4,4-difluorocyclohexyl)-2- |
| | | (methoxymethyl)pyrazolo[1,5-a]pyrimidine-3,7- |
| | | dicarboxamide |
| 81 | D | N7-(cyclopropylmethyl)-2-(methoxymethyl)pyrazolo[1,5- |
| | | a]pyrimidine-3,7-dicarboxamide |
| 82 | E | N7-(7-fluorotetralin-2-yl)-2-(methoxymethyl)pyrazolo[1,5- |
| | | a]pyrimidine-3,7-dicarboxamide |
| 83 | E | N7-[(2,4-difluorophenyl)methyl]-2- |
| | | (methoxymethyl)pyrazolo[1,5-a]pyrimidine-3,7- |
| | | dicarboxamide |
| 84 | C | 2-(methoxymethyl)-N7-(cis-4- |
| | | methylcyclohexyl)pyrazolo[1,5-a]pyrimidine-3,7- |
| | | dicarboxamide |
| 85 | E | N7-(5,6-dihydro-4H-cyclopenta[c]thiophen-5-yl)-2- |
| | | (methoxymethyl)pyrazolo[1,5-a]pyrimidine-3,7- |
| | | dicarboxamide |
| 86 | D | 2-(methoxymethyl)-N7-(trans-3- |
| | | methylcyclobutyl)pyrazolo[1,5-a]pyrimidine-3,7- |
| | | dicarboxamide |
| 87 | E | N7-(5-chloroindan-2-yl)-2-(methoxymethyl)pyrazolo[1,5- |
| | | a]pyrimidine-3,7-dicarboxamide |
| 88 | E | N7-(7-bicyclo[4.2.0]octa-1,3,5-trienyl)-2- |
| | | (methoxymethyl)pyrazolo[1,5-a]pyrimidine-3,7- |
| | | dicarboxamide |
| 89 | A | 2-(methoxymethyl)-7-[4-[3-(trifluoromethyl)-2- |
| | | pyridyl]piperazine-1-carbonyl]pyrazolo[1,5-a]pyrimidine-3- |
| | | carboxamide |
| 90 | A | N7-[[1-(dimethylamino)cyclohexyl]methyl]-2- |
| | | (methoxymethyl)pyrazolo[1,5-a]pyrimidine-3,7- |
| | | dicarboxamide |
| 91 | E | N7-(4-fluoroindan-2-yl)-2-(methoxymethyl)pyrazolo[1,5- |
| | | a]pyrimidine-3,7-dicarboxamide |
| 92 | E | N7-(4,6-difluoroindan-2-yl)-2-(methoxymethyl)pyrazolo[1,5- |
| | | a]pyrimidine-3,7-dicarboxamide |
| 93 | E | 2-(methoxymethyl)-N7-(1,2,3,4-tetrahydroquinolin-3- |
| | | yl)pyrazolo[1,5-a]pyrimidine-3,7-dicarboxamide |
| 94 | D | N7-(trans-3-fluoro-3-methyl-cyclobutyl)-2- |
| | | (methoxymethyl)pyrazolo[1,5-a]pyrimidine-3,7- |
| | | dicarboxamide |
| 95 | C | 7-(2,3-dihydro-1H-isoindole-2-carbonyl)-2- |
| | | (methoxymethyl)pyrazolo[1,5-a]pyrimidine-3-carboxamide |
| 96 | B | N7-[2-(3-methylphenoxy)butyl]pyrazolo[1,5-a]pyrimidine- |
| | | 3,7-dicarboxamide |
| 97 | C | N7-[2-methyl-5-(pyridin-4-yl)phenyl]pyrazolo[1,5- |
| | | a]pyrimidine-3,7-dicarboxamide |
| 98 | B | N7-[1-(5-methoxy-1H-1,3-benzodiazol-2- |
| | | yl)ethyl]pyrazolo[1,5-a]pyrimidine-3,7-dicarboxamide |
| 99 | B | N7-[1-(4-tert-butylphenoxy)propan-2-yl]pyrazolo[1,5- |
| | | a]pyrimidine-3,7-dicarboxamide |
| 100 | B | N7-(3,3,3-trifluoropropyl)pyrazolo[1,5-a]pyrimidine-3,7- |
| | | dicarboxamide |
| 101 | C | N7-[(4,4-difluorocyclohexyl)methyl]pyrazolo[1,5- |
| | | a]pyrimidine-3,7-dicarboxamide |
| 102 | C | N7-(3,3-difluorocyclobutyl)pyrazolo[1,5-a]pyrimidine-3,7- |
| | | dicarboxamide |
| 103 | C | N7-(4,4-difluorocyclohexyl)pyrazolo[1,5-a]pyrimidine-3,7- |
| | | dicarboxamide |
| 104 | D | N8-(6,7-dihydro-5H-cyclopenta[b]pyridin-6-yl)-2- |
| | | (methoxymethyl)imidazo[1,2-b]pyridazine-3,8- |
| | | dicarboxamide |
| 105 | E | N8-(5,6-dihydro-4H-cyclopenta[c]thiophen-5-yl)-2- |
| | | (methoxymethyl)imidazo[1,2-b]pyridazine-3,8- |
| | | dicarboxamide |
| 106 | E | N8-(7-bicyclo[4.2.0]octa-1,3,5-trienyl)-2- |
| | | (methoxymethyl)imidazo[1,2-b]pyridazine-3,8- |
| | | dicarboxamide |
| 107 | E | N8-(3,3-difluoroindan-1-yl)-2-(methoxymethyl)imidazo[1,2- |
| | | b]pyridazine-3,8-dicarboxamide |
| 108 | E | 2-(methoxymethyl)-N8-[(4S)-7-fluorochroman-4- |
| | | yl]imidazo[1,2-b]pyridazine-3,8-dicarboxamide |
| 109 | E | N8-(4,4-difluorocyclohexyl)-2-(methoxymethyl)imidazo[1,2- |
| | | b]pyridazine-3,8-dicarboxamide |
| 110 | E | N8-[(4-fluorophenyl)methyl]-2-(methoxymethyl)imidazo[1,2- |
| | | b]pyridazine-3,8-dicarboxamide |
| 111 | E | 2-cyclopropyl-N7-[cis-3- |
| | | (trifluoromethoxy)cyclobutyl]pyrazolo[1,5-a]pyrimidine-3,7- |
| | | dicarboxamide |
| 112 | E | 2-cyclopropyl-N7-(3,3-difluoroindan-1-yl)pyrazolo[1,5- |
| | | a]pyrimidine-3,7-dicarboxamide |
| 113 | E | 2-cyclopropyl-N7-[(4S)-7-fluorochroman-4-yl]pyrazolo[1,5- |
| | | a]pyrimidine-3,7-dicarboxamide |
| 114 | E | N7-(6-chlorochroman-3-yl)-2-(methoxymethyl)pyrazolo[1,5- |
| | | a]pyrimidine-3,7-dicarboxamide |
| 115 | E | 2-cyclopropyl-N7-(6,7-dihydro-5H-cyclopenta[b]pyridin-6- |
| | | yl)pyrazolo[1,5-a]pyrimidine-3,7-dicarboxamide |
| 116 | E | N7-(4,4-difluorotetralin-1-yl)-2- |
| | | (methoxymethyl)pyrazolo[1,5-a]pyrimidine-3,7- |
| | | dicarboxamide |
| 117 | E | N7-(8-fluoro-3-quinolyl)-2-(methoxymethyl)pyrazolo[1,5- |
| | | a]pyrimidine-3,7-dicarboxamide |
| 118 | E | 2-(methoxymethyl)-N7-(1-methylindol-2-yl)pyrazolo[1,5- |
| | | a]pyrimidine-3,7-dicarboxamide |
| 119 | E | 2-(methoxymethyl)-N7-[(3R)-6,7-difluoro-2,3- |
| | | dihydrobenzofuran-3-yl]pyrazolo[1,5-a]pyrimidine-3,7- |
| | | dicarboxamide |
| 120 | E | N7-[(3R)-5-fluoro-2,3-dihydrobenzofuran-3-yl]-2- |
| | | (methoxymethyl)pyrazolo[1,5-a]pyrimidine-3,7- |
| | | dicarboxamide |
| 121 | D | 2-(methoxymethyl)-N7-(2-methylindan-2-yl)pyrazolo[1,5- |
| | | a]pyrimidine-3,7-dicarboxamide |
| 122 | D | N7-[1-(4-fluorophenyl)ethyl]-2- |
| | | (methoxymethyl)pyrazolo[1,5-a]pyrimidine-3,7- |
| | | dicarboxamide |
| 123 | E | 2-(methoxymethyl)-N7-[(4S)-7-fluorochroman-4- |
| | | yl]pyrazolo[1,5-a]pyrimidine-3,7-dicarboxamide |
| 124 | C | N7-(2,2-difluoro-6-methyl-spiro[3.3]heptan-6-yl)-2- |
| | | (methoxymethyl)pyrazolo[1,5-a]pyrimidine-3,7- |
| | | dicarboxamide |
| 125 | C | 2-(methoxymethyl)-N7-[2,2,2-trifluoro-1-(4- |
| | | fluorophenyl)ethyl]pyrazolo[1,5-a]pyrimidine-3,7- |
| | | dicarboxamide |
| 126 | D | N7-(2,2-difluorospiro[3.3]heptan-6-yl)-2- |
| | | (methoxymethyl)pyrazolo[1,5-a]pyrimidine-3,7- |
| | | dicarboxamide |
| 127 | D | N7-[1-(4-fluorophenyl)cyclopropyl]-2- |
| | | (methoxymethyl)pyrazolo[1,5-a]pyrimidine-3,7- |
| | | dicarboxamide |
| 128 | E | 2-(methoxymethyl)-N7-[(3R)-6-fluoro-2,3- |
| | | dihydrobenzofuran-3-yl]pyrazolo[1,5-a]pyrimidine-3,7- |
| | | dicarboxamide |
| 129 | E | N7-(3,3-difluoroindan-1-yl)-2-(methoxymethyl)pyrazolo[1,5- |
| | | a]pyrimidine-3,7-dicarboxamide |
| 130 | E | 2-cyclopropyl-N7-indan-2-yl-pyrazolo[1,5-a]pyrimidine-3,7- |
| | | dicarboxamide |
| 131 | C | N7-(2,2-difluorospiro[3.3]heptan-7-yl)-2- |
| | | (methoxymethyl)pyrazolo[1,5-a]pyrimidine-3,7- |
| | | dicarboxamide |
| 132 | D | 2-(methoxymethyl)-N7-[cis-4- |
| | | (trifluoromethoxy)cyclohexyl]pyrazolo[1,5-a]pyrimidine-3,7- |
| | | dicarboxamide |
| 133 | E | N7-[(4,4-difluorocyclohexyl)methyl]-2- |
| | | (methoxymethyl)pyrazolo[1,5-a]pyrimidine-3,7- |
| | | dicarboxamide |
| 134 | E | 2-cyclopropyl-N7-(5,6-dihydro-4H-cyclopenta[c]thiophen-5- |
| | | yl)pyrazolo[1,5-a]pyrimidine-3,7-dicarboxamide |
| 135 | E | 2-cyclopropyl-N7-(4,4-difluorocyclohexyl)pyrazolo[1,5- |
| | | a]pyrimidine-3,7-dicarboxamide |
| 136 | E | 2-cyclopropyl-N7-(3,3-difluorocyclobutyl)pyrazolo[1,5- |
| | | a]pyrimidine-3,7-dicarboxamide |
| 137 | E | 2-propyl-N7-[cis-3- |
| | | (trifluoromethoxy)cyclobutyl]pyrazolo[1,5-a]pyrimidine-3,7- |
| | | dicarboxamide |
| 138 | E | N7-(7-bicyclo[4.2.0]octa-1,3,5-trienyl)-2-cyclopropyl- |
| | | pyrazolo[1,5-a]pyrimidine-3,7-dicarboxamide |
| 139 | E | N7-indan-2-yl-2-propyl-pyrazolo[1,5-a]pyrimidine-3,7- |
| | | dicarboxamide |
| 140 | E | 2-cyclopropyl-N7-[(4-fluorophenyl)methyl]pyrazolo[1,5- |
| | | a]pyrimidine-3,7-dicarboxamide |
| 141 | E | 2-(hydroxymethyl)-N7-[cis-3- |
| | | (trifluoromethoxy)cyclobutyl]pyrazolo[1,5-a]pyrimidine-3,7- |
| | | dicarboxamide |
| 142 | E | 2-(cyclopropylmethoxy)-N7-indan-2-yl-pyrazolo[1,5- |
| | | a]pyrimidine-3,7-dicarboxamide |
| 143 | D | 7-(indan-2-ylcarbamoyl)-2-(methoxymethyl)pyrazolo[1,5- |
| | | a]pyrimidine-3-carboxylic acid |
| 144 | E | N7-(6-bromochroman-3-yl)-2-(methoxymethyl)pyrazolo[1,5- |
| | | a]pyrimidine-3,7-dicarboxamide |
| 145 | E | 2-(methylamino)-N7-[cis-3- |
| | | (trifluoromethoxy)cyclobutyl]pyrazolo[1,5-a]pyrimidine-3,7- |
| | | dicarboxamide |
| 146 | D | N7-(6,7-dihydro-5H-cyclopenta[b]pyridin-6-yl)-2- |
| | | (methoxymethyl)pyrazolo[1,5-a]pyrimidine-3,7- |
| | | dicarboxamide |
| 147 | E | N7-(6,7-dihydro-5H-cyclopenta[b]pyridin-6-yl)-2- |
| | | (methoxymethyl)pyrazolo[1,5-a]pyrimidine-3,7- |
| | | dicarboxamide |
| 148 | E | 7-(indan-2-ylcarbamothioyl)-2- |
| | | (methoxymethyl)pyrazolo[1,5-a]pyrimidine-3-carboxamide |
| 149 | D | 3-carbamothioyl-N-indan-2-yl-2- |
| | | (methoxymethyl)pyrazolo[1,5-a]pyrimidine-7-carboxamide |
| 150 | E | 2-cyclopropyl-N8-(4,4-difluorocyclohexyl)imidazo[1,2- |
| | | b]pyridazine-3,8-dicarboxamide |
| 151 | E | 2-cyclopropyl-N8-[cis-3- |
| | | (trifluoromethoxy)cyclobutyl]imidazo[1,2-b]pyridazine-3,8- |
| | | dicarboxamide |
| 152 | E | N7-(1,1-difluoroindan-2-yl)-2-(methoxymethyl)pyrazolo[1,5- |
| | | a]pyrimidine-3,7-dicarboxamide |
| 153 | E | N7-(4,6-difluoro-2,3-dihydrobenzofuran-3-yl)-2- |
| | | (methoxymethyl)pyrazolo[1,5-a]pyrimidine-3,7- |
| | | dicarboxamide |
| 154 | D | 2-(methoxymethyl)-N7-(2,4,4- |
| | | trifluorocyclohexyl)pyrazolo[1,5-a]pyrimidine-3,7- |
| | | dicarboxamide |
| 155 | D | 2-(methoxymethyl)-N7-(1-methylindan-1-yl)pyrazolo[1,5- |
| | | a]pyrimidine-3,7-dicarboxamide |
| 156 | D | 7-cyclopropyl-N4-(4,4-difluorocyclohexyl)pyrazolo[5,1- |
| | | c][1,2,4]triazine-4,8-dicarboxamide |
| 157 | E | 2-(trideuteriomethoxymethyl)-N7-[cis-3- |
| | | (trifluoromethoxy)cyclobutyl]pyrazolo[1,5-a]pyrimidine-3,7- |
| | | dicarboxamide |
| 158 | E | 2-(fluoromethoxymethyl)-N7-[cis-3- |
| | | (trifluoromethoxy)cyclobutyl]pyrazolo[1,5-a]pyrimidine-3,7- |
| | | dicarboxamide |
| 159 | E | N7-indan-2-yl-2-(methylamino)pyrazolo[1,5-a]pyrimidine- |
| | | 3,7-dicarboxamide |
| 160 | E | N7-(6,7-dihydro-5H-cyclopenta[b]pyridin-6-yl)-2- |
| | | (trideuteriomethoxymethyl)pyrazolo[1,5-a]pyrimidine-3,7- |
| | | dicarboxamide |
| 161 | C | 2-(methoxymethyl)-N3-methyl-N7-[cis-3- |
| | | (trifluoromethoxy)cyclobutyl]pyrazolo[1,5-a]pyrimidine-3,7- |
| | | dicarboxamide |
| 162 | D | 2-(cyclopropylmethoxy)-N7-[cis-3- |
| | | (trifluoromethoxy)cyclobutyl]pyrazolo[1,5-a]pyrimidine-3,7- |
| | | dicarboxamide |
| 163 | E | 2-cyclopropyl-N8-(6,7-dihydro-5H-cyclopenta[b]pyridin-6- |
| | | yl)imidazo[1,2-b]pyridazine-3,8-dicarboxamide |
| 164 | E | N8-(4,6-difluoroindan-2-yl)-2-(methoxymethyl)imidazo[1,2- |
| | | b]pyridazine-3,8-dicarboxamide |
| 165 | D | N7-(6,7-dihydro-5H-pyrrolo[1,2-a]imidazol-6-yl)-2- |
| | | (methoxymethyl)pyrazolo[1,5-a]pyrimidine-3,7- |
| | | dicarboxamide |
| 166 | E | N7-[(4R)-7-fluorochroman-4-yl]-2- |
| | | (methoxymethyl)pyrazolo[1,5-a]pyrimidine-3,7- |
| | | dicarboxamide |
| 167 | E | N7-[(3S)-6-fluoro-2,3-dihydrobenzofuran-3-yl]-2- |
| | | (methoxymethyl)pyrazolo[1,5-a]pyrimidine-3,7- |
| | | dicarboxamide |
| 168 | E | N7-(1,1-dioxo-2,3-dihydrobenzothiophen-3-yl)-2- |
| | | (methoxymethyl)pyrazolo[1,5-a]pyrimidine-3,7- |
| | | dicarboxamide |
| 169 | E | N7-[(3R)-7-chloro-2,3-dihydrobenzofuran-3-yl]-2- |
| | | (methoxymethyl)pyrazolo[1,5-a]pyrimidine-3,7- |
| | | dicarboxamide |
| 170 | E | N7-(6,7-dihydro-5H-cyclopenta[c]pyridin-6-yl)-2- |
| | | (methoxymethyl)pyrazolo[1,5-a]pyrimidine-3,7- |
| | | dicarboxamide |
| 171 | D | N7-(3,4-dihydro-2H-pyrano[2,3-b]pyridin-4-yl)-2- |
| | | (methoxymethyl)pyrazolo[1,5-a]pyrimidine-3,7- |
| | | dicarboxamide |
| 172 | D | N7-[(4S)-chroman-4-yl]-2-(methoxymethyl)pyrazolo[1,5- |
| | | a]pyrimidine-3,7-dicarboxamide |
| 173 | E | N7-(3,3-difluoroindan-1-yl)-2-(methoxymethyl)pyrazolo[1,5- |
| | | a]pyrimidine-3,7-dicarboxamide |
| 174 | E | N7-(3,3-difluoroindan-1-yl)-2-(methoxymethyl)pyrazolo[1,5- |
| | | a]pyrimidine-3,7-dicarboxamide |
| 175 | E | N7-[-4,4-difluorotetralin-1-yl]-2- |
| | | (methoxymethyl)pyrazolo[1,5-a]pyrimidine-3,7- |
| | | dicarboxamide |
| 176 | E | N7-[-4,4-difluorotetralin-1-yl]-2- |
| | | (methoxymethyl)pyrazolo[1,5-a]pyrimidine-3,7- |
| | | dicarboxamide |
| 177 | E | N7-(1,1-dioxo-2,3-dihydrobenzothiophen-3-yl)-2- |
| | | (methoxymethyl)pyrazolo[1,5-a]pyrimidine-3,7- |
| | | dicarboxamide |
| 178 | E | N7-[(3R)-7-chloro-2,3-dihydrobenzofuran-3-yl]-2- |
| | | (methoxymethyl)pyrazolo[1,5-a]pyrimidine-3,7- |
| | | dicarboxamide |
| 179 | E | N7-(6,7-dihydro-5H-cyclopenta[c]pyridin-6-yl)-2- |
| | | (methoxymethyl)pyrazolo[1,5-a]pyrimidine-3,7- |
| | | dicarboxamide |
| 180 | D | N7-(3,4-dihydro-2H-pyrano[2,3-b]pyridin-4-yl)-2- |
| | | (methoxymethyl)pyrazolo[1,5-a]pyrimidine-3,7- |
| | | dicarboxamide |
| 181 | D | N7-[(4S)-chroman-4-yl]-2-(methoxymethyl)pyrazolo[1,5- |
| | | a]pyrimidine-3,7-dicarboxamide |
| 182 | E | N7-(6,7-difluorochroman-4-yl)-2- |
| | | (methoxymethyl)pyrazolo[1,5-a]pyrimidine-3,7- |
| | | dicarboxamide |
| 183 | E | 2-(methoxymethyl)-N7-[(1S)-indan-1-yl]pyrazolo[1,5- |
| | | a]pyrimidine-3,7-dicarboxamide |
| 184 | E | N7-(2,3-dihydrofuro[2,3-b]pyridin-3-yl)-2- |
| | | (methoxymethyl)pyrazolo[1,5-a]pyrimidine-3,7- |
| | | dicarboxamide |
| 185 | D | N7-(5,7-difluorochroman-3-yl)-2- |
| | | (methoxymethyl)pyrazolo[1,5-a]pyrimidine-3,7- |
| | | dicarboxamide |
| 186 | C | N7-(4,4-dimethyltetralin-1-yl)-2- |
| | | (methoxymethyl)pyrazolo[1,5-a]pyrimidine-3,7- |
| | | dicarboxamide |
| 187 | C | 2-(methoxymethyl)-N7-(5,6,7,8-tetrahydroimidazo[1,2- |
| | | a]pyridin-6-yl)pyrazolo[1,5-a]pyrimidine-3,7-dicarboxamide |
| 188 | C | 2-(methoxymethyl)-N7-(5,6,7,8-tetrahydroimidazo[1,5- |
| | | a]pyridin-6-yl)pyrazolo[1,5-a]pyrimidine-3,7-dicarboxamide |
| 189 | E | N7-(1,1-dioxo-3,4-dihydro-2H-thiochromen-4-yl)-2- |
| | | (methoxymethyl)pyrazolo[1,5-a]pyrimidine-3,7- |
| | | dicarboxamide |
| 190 | D | 2-(methoxymethyl)-N7-(4,5,6,7-tetrahydro-1,3- |
| | | benzothiazol-6-yl)pyrazolo[1,5-a]pyrimidine-3,7- |
| | | dicarboxamide |
| 191 | E | N7-(5,7-difluoro-2,3-dihydrobenzofuran-3-yl)-2- |
| | | (methoxymethyl)pyrazolo[1,5-a]pyrimidine-3,7- |
| | | dicarboxamide |
| 192 | E | N7-(7-fluoro-2,3-dihydrobenzofuran-3-yl)-2- |
| | | (methoxymethyl)pyrazolo[1,5-a]pyrimidine-3,7- |
| | | dicarboxamide |
| 193 | E | 2-(methoxymethyl)-N7-[(1S)-tetralin-1-yl]pyrazolo[1,5- |
| | | a]pyrimidine-3,7-dicarboxamide |
| 194 | E | N7-(5-fluoroindan-2-yl)-2-(methoxymethyl)pyrazolo[1,5- |
| | | a]pyrimidine-3,7-dicarboxamide |
| 195 | E | N7-(5,6-difluoroindan-2-yl)-2-(methoxymethyl)pyrazolo[1,5- |
| | | a]pyrimidine-3,7-dicarboxamide |
| 196 | E | N7-(6-fluorochroman-3-yl)-2-(methoxymethyl)pyrazolo[1,5- |
| | | a]pyrimidine-3,7-dicarboxamide |
| 197 | C | N7-[(4-hydroxyphenyl)methyl]-2- |
| | | (methoxymethyl)pyrazolo[1,5-a]pyrimidine-3,7- |
| | | dicarboxamide |
| 198 | D | 2-(methoxymethyl)-N7-[(1S)-3,3-dimethylindan-1- |
| | | yl]pyrazolo[1,5-a]pyrimidine-3,7-dicarboxamide |
| 199 | D | 2-(methoxymethyl)-N7-[(2- |
| | | methoxyphenyl)methyl]pyrazolo[1,5-a]pyrimidine-3,7- |
| | | dicarboxamide |
| 200 | E | N7-(cis-1-hydroxyindan-2-yl)-2- |
| | | (methoxymethyl)pyrazolo[1,5-a]pyrimidine-3,7- |
| | | dicarboxamide |
| 201 | E | 2-(methoxymethyl)-N7-cis-1-methylindan-2-yl]pyrazolo[1,5- |
| | | a]pyrimidine-3,7-dicarboxamide |
| 202 | E | 2-(ethoxymethyl)-N7-indan-2-yl-pyrazolo[1,5-a]pyrimidine- |
| | | 3,7-dicarboxamide |
| 203 | D | 2-tetrahydrofuran-2-yl-N7-[cis-3- |
| | | (trifluoromethoxy)cyclobutyl]pyrazolo[1,5-a]pyrimidine-3,7- |
| | | dicarboxamide |
| 204 | D | 2-(hydroxymethyl)-N7-indan-2-yl-pyrazolo[1,5-a]pyrimidine- |
| | | 3,7-dicarboxamide |
| 205 | D | 2-(ethoxymethyl)-N7-[cis-3- |
| | | (trifluoromethoxy)cyclobutyl]pyrazolo[1,5-a]pyrimidine-3,7- |
| | | dicarboxamide |
| 206 | D | 2-(methoxymethyl)-5-methyl-N7-[cis-3- |
| | | (trifluoromethoxy)cyclobutyl]pyrazolo[1,5-a]pyrimidine-3,7- |
| | | dicarboxamide |
| 207 | C | N7-(3,3-difluorocyclobutyl)-2-(hydroxymethyl)pyrazolo[1,5- |
| | | a]pyrimidine-3,7-dicarboxamide |
| 208 | E | 2-cyclopropyl-N8-(5,6-dihydro-4H-cyclopenta[c]thiophen-5- |
| | | yl)imidazo[1,2-b]pyridazine-3,8-dicarboxamide |
| 209 | E | 2-cyclopropyl-N8-(3,3-difluoroindan-1-yl)imidazo[1,2- |
| | | b]pyridazine-3,8-dicarboxamide |
| 210 | E | N8-(7-bicyclo[4.2.0]octa-1,3,5-trienyl)-2-cyclopropyl- |
| | | imidazo[1,2-b]pyridazine-3,8-dicarboxamide |
| 211 | E | 2-cyclopropyl-N8-[(4S)-7-fluorochroman-4-yl]imidazo[1,2- |
| | | b]pyridazine-3,8-dicarboxamide |
| 212 | D | N4-(4,4-difluorocyclohexyl)-6-(methoxymethyl)imidazo[2,1- |
| | | f][1,2,4]triazine-4,7-dicarboxamide |
| 213 | D | N7-(3,3-difluorocyclobutyl)-2-(1-hydroxy-1-methyl- |
| | | ethyl)pyrazolo[1,5-a]pyrimidine-3,7-dicarboxamide |
| 214 | E | N7-indan-2-yl-2-(methylsulfanylmethyl)pyrazolo[1,5- |
| | | a]pyrimidine-3,7-dicarboxamide |
| 215 | D | N7-indan-2-yl-2-(methylsulfonylmethyl)pyrazolo[1,5- |
| | | a]pyrimidine-3,7-dicarboxamide |
| 216 | E | 2-(cyclopropoxymethyl)-N7-[cis-3- |
| | | (trifluoromethoxy)cyclobutyl]pyrazolo[1,5-a]pyrimidine-3,7- |
| | | dicarboxamide |
| 217 | E | 2-(methylsulfanylmethyl)-N7-[cis-3- |
| | | (trifluoromethoxy)cyclobutyl]pyrazolo[1,5-a]pyrimidine-3,7- |
| | | dicarboxamide |
| 218 | D | N8-(4,4-difluorocyclohexyl)-2-(methoxymethyl)-6-methyl- |
| | | imidazo[1,2-b]pyridazine-3,8-dicarboxamide |
| 219 | E | 2-(methoxymethyl)-N8-[(4R)-1,1-dioxo-3,4-dihydro-2H- |
| | | thiochromen-4-yl]imidazo[1,2-b]pyridazine-3,8- |
| | | dicarboxamide |
| 220 | D | 2-(methoxymethyl)-N8-[(4S)-1,1-dioxo-3,4-dihydro-2H- |
| | | thiochromen-4-yl]imidazo[1,2-b]pyridazine-3,8- |
| | | dicarboxamide |
| 221 | D | N8-(1,1-dioxo-3,4-dihydro-2H-thiochromen-4-yl)-2- |
| | | (methoxymethyl)imidazo[1,2-b]pyridazine-3,8- |
| | | dicarboxamide |
| 222 | E | 2-cyclopropyl-N8-(1,1-dioxo-2,3-dihydrobenzothiophen-3- |
| | | yl)imidazo[1,2-b]pyridazine-3,8-dicarboxamide |
| 223 | E | N8-(1,1-dioxo-2,3-dihydrobenzothiophen-3-yl)-2- |
| | | (methoxymethyl)imidazo[1,2-b]pyridazine-3,8- |
| | | dicarboxamide |
| 224 | D | 2-(methoxymethyl)-N7-[trans-3- |
| | | (trifluoromethoxy)cyclobutyl]pyrazolo[1,5-a]pyrimidine-3,7- |
| | | dicarboxamide |
| 225 | E | N7-(1,1-dioxothietan-3-yl)-2-(methoxymethyl) pyrazolo[1,5- |
| | | a] pyrimidine-3,7-dicarboxamide |
| 226 | E | N7-(4,4-difluorotetralin-1-yl)-2-(methoxymethyl)-5-methyl- |
| | | pyrazolo[1,5-a]pyrimidine-3,7-dicarboxamide |
| 227 | E | N7-indan-2-yl-2-(methoxymethyl)-5-methyl-pyrazolo[1,5- |
| | | a]pyrimidine-3,7-dicarboxamide |
| 228 | E | N7-(1,1-dioxo-2,3-dihydrobenzothiophen-3-yl)-2-isopropyl- |
| | | pyrazolo[1,5-a]pyrimidine-3,7-dicarboxamide |
| 229 | E | 2-cyclopropyl-N7-(1,1-dioxo-3,4-dihydro-2H-thiochromen- |
| | | 4-yl)pyrazolo[1,5-a]pyrimidine-3,7-dicarboxamide |
| 230 | E | N7-(1,1-dioxo-3,4-dihydro-2H-thiochromen-4-yl)-2- |
| | | isopropyl-pyrazolo[1,5-a]pyrimidine-3,7-dicarboxamide |
| 231 | E | 2-cyclopropyl-N7-(1,1-dioxo-2,3-dihydrobenzothiophen-3- |
| | | yl)pyrazolo[1,5-a]pyrimidine-3,7-dicarboxamide |
| 232 | E | N7-(3,3-difluoroindan-1-yl)-2-(methoxymethyl)-5-methyl- |
| | | pyrazolo[1,5-a]pyrimidine-3,7-dicarboxamide |
| 233 | E | N7-[(4R)-1,1-dioxo-3,4-dihydro-2H-thiochromen-4-yl]-2- |
| | | (methoxymethyl)pyrazolo[1,5-a]pyrimidine-3,7- |
| | | dicarboxamide |
| 234 | E | N7-[(4S)-1,1-dioxo-3,4-dihydro-2H-thiochromen-4-yl]-2- |
| | | (methoxymethyl)pyrazolo[1,5-a]pyrimidine-3,7- |
| | | dicarboxamide |
| 235 | E | 2-cyclopropyl-N7-[(4R)-1,1-dioxo-3,4-dihydro-2H- |
| | | thiochromen-4-yl]pyrazolo[1,5-a]pyrimidine-3,7- |
| | | dicarboxamide |
| 236 | E | 2-cyclopropyl-N7-[(4S)-1,1-dioxo-3,4-dihydro-2H- |
| | | thiochromen-4-yl] pyrazolo[1,5-a]pyrimidine-3,7- |
| | | dicarboxamide |
| 237 | E | 2-(methoxymethyl)-N7-(3,3,6-trifluoroindan-1-yl) |
| | | pyrazolo[1,5-a] pyrimidine-3,7-dicarboxamide |
| 238 | E | N7-[(3S)-6,7-difluoro-2,3-dihydrobenzofuran-3-yl]-2- |
| | | (methoxymethyl)pyrazolo[1,5-a]pyrimidine-3,7- |
| | | dicarboxamide |
| 239 | E | 2-(methoxymethyl)-N7-(4,4,6-trifluorotetralin-1- |
| | | yl)pyrazolo[1,5-a]pyrimidine-3,7-dicarboxamide |
| 240 | E | N7-(4,4-difluorocyclohexyl)-2-(methoxymethyl)-5-methyl- |
| | | pyrazolo[1,5-a]pyrimidine-3,7-dicarboxamide |
| 241 | D | N7-(1,1-dioxo-3,4-dihydro-2H-thiochromen-4-yl)-2- |
| | | (methoxymethyl)-5-methyl-pyrazolo[1,5-a]pyrimidine-3,7- |
| | | dicarboxamide |
| 242 | E | 2-cyclopropyl-N7-(1,1-dioxo-3,4-dihydro-2H-thiochromen- |
| | | 4-yl)-5-methyl-pyrazolo[1,5-a]pyrimidine-3,7-dicarboxamide |

hGPR68 IC₅₀: A = 50 μM to 100 μM; B = 5 μM to 50 μM; C = 500 nM to 5 μM; D = 50 nM to 500 nM; E = <50 nM

### CLAUSES AND PARAGRAPHS

Alternative expressions of the inventive concept are set out in each of the following numbered clauses:

Clause 1. A compound of formula (1):

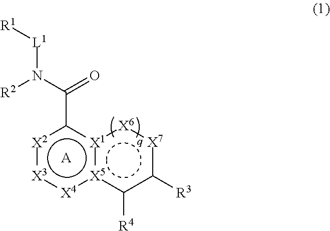

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein:
Q is O or S;
X¹ is selected from C and N;
X² is selected from CR⁵ and N;
X³ is selected from CR⁶ and N;
X⁴ is selected from CR⁷ and N;
X⁵ is selected from C and N;
wherein X¹, X², X³, X⁴ and X⁵ are selected so that the ring labelled A is aromatic;
X⁶ is selected from CR⁸R⁹, O and N;
X⁷ is selected from CR¹⁰, CR¹¹R¹², O and N;
q is 0 or 1;
wherein when q is 0, X⁷ is O or N; and when X⁷ is O or N and q is 1, X⁶ is CR⁸R⁹;

L¹ is a bond, alkylene, haloalkylene, cycloalkylene, or heterocycloalkyl;

R¹ is selected from alkyl, cycloalkyl, bicyclic cycloalkyl, heterocycloalkyl, bicyclic heterocycloalkyl, aryl, bicyclic aryl, heteroaryl, bicyclic heteroaryl, aryloxy, and heteroaryloxy, wherein said alkyl, cycloalkyl, bicyclic cycloalkyl, heterocycloalkyl, bicyclic heterocycloalkyl, aryl, bicyclic aryl, heteroaryl, bicyclic heteroaryl, aryloxy and heteroaryloxy are each optionally substituted by one or more substituents independently selected from halogen, OH, CN, NR^{1a}R^{1b}, alkyl, haloalkyl, alkoxy, haloalkoxy, alkyl-NR^{1a}R^{1b}, cycloalkyl, heterocycloakyl, aryl, heteroaryl, benzyl, aryloxy, haloaryloxy, heteroaryloxy, C₄ alkyl substituted by a heterocycloalkyl;
R^{1a} and R^{1b} are each independently selected from H and alkyl;
R² is selected from H, alkyl and cycloalkyl;
or R¹, R² and L¹ and the nitrogen atom to which R² and L¹ are attached form a heterocycloalkyl, bicyclic heterocycloalkyl, heteroaryl or bicyclic heteroaryl, wherein said heterocycloalkyl, bicyclic heterocycloalkyl, heteroaryl and bicyclic heteroaryl are each optionally substituted by one or more substituents independently selected from halogen, OH, CN, NR^{1a}R^{1b}, alkyl, haloalkyl, alkoxy, haloalkoxy and alkyl-NR^{1a}R^{1b};
R³ is selected from H, OH, CN, NR^{3a}R^{3b}, alkyl, cycloalkyl, heterocycloalkyl, aryl, heteroaryl, alkoxyalkyl, haloalkoxyalkyl, cycloalkoxyalkyl, cycloalkylalkoxy, alkyl-S-alkyl, and alkyl-SO₂-alkyl, wherein said alkyl, cycloalkyl, heterocycloalkyl, aryl and heteroaryl are each optionally substituted by one or more substituents independently selected from halogen, OH, CN, alkyl and NR^{3c}R^{3d};
R^{3a} is selected from H, alkyl, alkyl-OH, and alkyl-NR^{3e}R^{3f};
R^{3b}, R^{3c}, R^{3d}, R^{3e} and R^{3f} are each independently selected from H and alkyl;
R⁴ is selected from C(O)OH, C(O)NR^{4a}R^{4b}, C(S)NR^{4a}R^{4b} and heteroaryl;
R^{4a} and R^{4b} are each independently selected from H and alkyl;
R⁵ and R⁶ are each independently selected from H, halogen, OH, CN, alkyl, haloalkyl, alkoxy, alkoxyalkyl, cycloalkyl, heterocycloalkyl, aryl and heteroaryl;
or R⁵ and R⁶ together with the carbon atoms to which they are attached form a heterocycle; and
R⁷, R⁸, R⁹, R¹⁰, R¹¹ and R¹² are each independently selected from H, halogen, OH, CN, alkyl, haloalkyl, alkoxy, cycloalkyl, heterocycloalkyl, aryl and heteroaryl.

Clause 2. The compound of clause 1, wherein
L¹ is a bond, C₁-C₆ alkylene, C₁-C₆ haloalkylene, C₃-C₆ cycloalkylene, or a 5- or 6-membered heterocycloalkyl having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, C₉-C₁₀ bicyclic aryl, 5- or 6-membered heteroaryl having one or more ring members selected from N, O, S and SO₂, 8- to 10-membered bicyclic heteroaryl having one or more ring members selected from N, O, S and SO₂, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is selected from H, C₁-C₄ alkyl and C₃-C₈ cycloakyl; or R¹, R² and L¹ and the nitrogen atom to which R² and L¹ are attached form a 3- to 8-membered heterocycloalkyl having one or more heteroatoms selected from N, O and S, 8- to 11-membered bicyclic heterocycloalkyl having one or more heteroatoms selected from N, O and S, 5- or 6-membered heteroaryl having one or more heteroatoms selected from N, O and S, or 8- to 10-membered bicyclic heteroaryl having one or more heteroatoms selected from N, O and S, wherein said heterocycloalkyl, bicyclic heterocycloalkyl, heteroaryl and bicyclic heteroaryl are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy and C₁-C₄ alkyl-NR^{1a}R^{1b};
R³ is selected from H, OH, CN, NR^{3a}R^{3b}, C₁-C₄ alkyl, C₃-C₈ cycloalkyl, 3- to 8-membered heterocycloalkyl having one or more heteroatoms selected from N, O and S, phenyl, and 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, CN, C₁-C₄ alkyl, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} and R^{4b} are each independently selected from H and C₁-C₄ alkyl;
R⁵ and R⁶ are each independently selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S; or
R⁵ and R⁶ together with the carbon atoms to which they are attached form a 5- or 6-membered heterocycloalkyl having one or more heteroatoms selected from N, O and S; and
R⁷, R⁸, R⁹, R¹⁰, R¹¹, and R¹² are each independently selected from H, halogen, OH, CN, C₁-C₄ alkyl, C₁-C₄ haloalkyl, C₁-C₄ alkoxy, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S.

Clause 3. The compound of clause 1 or clause 2, wherein
L¹ is a bond, C₁-C₆ alkylene, C₁-C₆ haloalkylene, C₃-C₆ cycloalkylene, or a 5- or 6-membered heterocycloalkyl having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, C₉-C₁₀ bicyclic aryl, 5- or 6-membered heteroaryl having one or more ring members selected from N, O, S and SO₂, 8- to 10-membered bicyclic heteroaryl having one or more ring members selected from N, O, S and SO₂, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is selected from H, C₁-C₄ alkyl and C₃-C₈ cycloakyl;
R³ is selected from H, OH, CN, NR^{3a}R^{3b}, C₁-C₄ alkyl, C₃-C₈ cycloalkyl, 3- to 8-membered heterocycloalkyl having one or more heteroatoms selected from N, O and S, phenyl, and 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, CN, C₁-C₄ alkyl, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} and R^{4b} are each independently selected from H and C₁-C₄ alkyl;
R⁵ and R⁶ are each independently selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S; or
R⁵ and R⁶ together with the carbon atoms to which they are attached form a 5- or 6-membered heterocycloalkyl having one or more heteroatoms selected from N, O and S; and
R⁷, R⁸, R⁹, R¹⁰, R¹¹, and R¹² are each independently selected from H, halogen, OH, CN, C₁-C₄ alkyl, C₁-C₄ haloalkyl, C₁-C₄ alkoxy, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S.

Clause 4. The compound of any preceding clause, wherein
L¹ is a bond, C₁-C₆ alkylene, C₁-C₆ haloalkylene, C₃-C₆ cycloalkylene, or a 5- or 6-membered heterocycloalkyl having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, C₉-C₁₀ bicyclic aryl, 5- or 6-membered heteroaryl having one or more ring members selected from N, O, S and SO₂, 8- to 10-membered bicyclic heteroaryl having one or more ring members selected from N, O, S and SO₂, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, 5- or 6-membered heteroaryl having one or more heteroatoms selected from N, O and S, benzyl, halophenoxy, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is selected from H, C₁-C₄ alkyl and C₃-C₈ cycloakyl;
R³ is selected from H, OH, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, C₁-C₄ alkyl, and NR^{3c}R^{3d};
R^{3a} is selected from H, C₁-C₄ alkyl, and C₁-C₄ alkyl-NR^{3e}R^{3f};
R^{3b} is selected from H and C₁-C₄ alkyl;
R^{3c} is selected from H and C₁-C₄ alkyl;
R^{3d} is selected from C₁-C₄ alkyl;
R^{3e} and R^{3f} are each independently selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ and R⁶ are each independently selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S; or
R⁵ and R⁶ together with the carbon atoms to which they are attached form a 5- or 6-membered heterocycloalkyl having one or more heteroatoms selected from N, O and S; and
R⁷, R⁸, R⁹, R¹⁰, R¹¹, and R¹² are each independently selected from H, halogen, OH, CN, C₁-C₄ alkyl, C₁-C₄ haloalkyl, C₁-C₄ alkoxy, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S.

Clause 5. The compound of any preceding clause, wherein
L¹ is a bond, C₁-C₆ alkylene, C₁-C₆ haloalkylene, C₃-C₆ cycloalkylene, or a 5- or 6-membered heterocycloalkyl having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, 8- to 10-membered bicyclic heteroaryl having one or more ring members selected from N, O, S and SO₂, and phenoxy, wherein said alkyl, cycloalkyl, heterocycloalkyl, bicyclic cycloalkyl, bicyclic heterocycloalkyl, phenyl, heteroaryl, bicyclic heteroaryl, and phenoxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, 5- or 6-membered heteroaryl having one or more heteroatoms selected from N, O and S, benzyl, halophenoxy, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, C₁-C₄ alkyl, and NR^{3c}R^{3d}:
R^{3a} is selected from H, C₁-C₄ alkyl, and C₁-C₄ alkyl-NR^{3e}R^{3f};
R^{3b} is selected from H and C₁-C₄ alkyl;
R^{3c} is selected from H and C₁-C₄ alkyl;
R^{3d} is selected from C₁-C₄ alkyl;
R^{3e} and R^{3f} are each independently selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is HR⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

Clause 6. The compound of any preceding clause, wherein
L¹ is a bond, C₁-C₆ alkylene, C₁-C₆ haloalkylene, C₃-C₆ cycloalkylene, or a 5- or 6-membered heterocycloalkyl having one or more heteroatoms selected from N, O and S;
R¹ selected from:
   C₁-C₄ haloalkyl;
   C₃-C₈ cycloalkyl optionally substituted by one or more substituents independently selected from halogen, OH, CN, NR^{1a}R^{1b}, C₁-C₄ alkyl, C₁-C₄ alkoxy, and C₁-C₄ haloalkoxy;
   C₇-C₁₁ bicyclic cycloalkyl optionally substituted by one or more substituents independently selected from halogen, OH, C₁-C₄ alkyl, C₁-C₄ haloalkyl, C₁-C₄ alkoxy, benzyl, C₃-C₈ cycloalkyl, halophenoxy, 5- or 6-membered heteroaryl having one or more heteroatoms selected from N, O and S, and C₁-C₄ alkyl substituted by a 5- or 6-membered heterocycloalkyl having one or more heteroatoms selected from N, O and S;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, OH, C₁-C₄ alkyl, C₁-C₄ alkoxy, C₁-C₄ alkyl-NR^{1a}R^{1b}, and 5- or 6-membered heteroaryl having one or more heteroatoms selected from N, O and S;
   5- or 6-membered heteroaryl having one or more ring members selected from N, O, S and SO₂, optionally substituted by one or more substituents independently selected from C₁-C₄ alkyl and C₁-C₄ haloalkyl;
   8- to 10-membered bicyclic heteroaryl having one or more ring members selected from N, O, S and SO₂, optionally substituted by one or more substitutents independently selected from halogen, C₁-C₄ alkyl and C₁-C₄ alkoxy; and phenoxy optionally substituted by one or more C₁-C₄ alkyl;
R^{1a} and R^{1b} are each independently selected from H and C₁-C₄ alkyl;
R² is H;
R³ is selected from:
   H;
   CN;
   NR^{3a}R^{3b};
   C₁-C₄ alkyl optionally substituted by one of more substituents selected from OH and NR^{3c}R^{3d};
   C₃-C₈ cycloalkyl;
   3- to 8-membered heterocycloalkyl having one or more heteroatoms selected from N, O and S, optionally substituted by one or more C₁-C₄ alkyl;
   phenyl optionally substituted by one or more halogen;
   5- or 6-membered heteroaryl having one or more heteroatoms selected from N, O and S;
   C₁-C₄ alkoxy-C₁-C₄ alkyl;
   C₁-C₄ haloalkoxy-C₁-C₄ alkyl;
   C₃-C₈ cycloalkoxy-C₁-C₄ alkyl;
   C₃-C₈ cycloalkyl-C₁-C₄ alkoxy;
   C₁-C₄ alkyl-S—C₁-C₄ alkyl; and
   C₁-C₄ alkyl-SO₂-C₁-C₄ alkyl;
R^{3a} is selected from H, C₁-C₄ alkyl, and C₁-C₄ alkyl-NR^{3e}R^{3f};
R^{3b} is selected from H and C₁-C₄ alkyl;
R^{3c} is selected from H and C₁-C₄ alkyl;
R^{3d} is selected from C₁-C₄ alkyl;
R^{3e} and R^{3f} are each independently selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

Clause 7. The compound of any preceding clause, wherein
L¹ is a bond, C₁-C₆ alkylene, C₁-C₆ haloalkylene, C₃-C₆ cycloalkylene, or a 5- or 6-membered heterocycloalkyl having one or more heteroatoms selected from N, O and S;
R¹ selected from:
   C₁-C₄ haloalkyl;
   C₃-C₈ cycloalkyl optionally substituted by one or more substituents independently selected from halogen, OH, CN, NR^{1a}R^{1b}, C₁-C₄ alkyl, C₁-C₄ alkoxy, and C₁-C₄ haloalkoxy;
   C₇-C₁₁ bicyclic cycloalkyl optionally substituted by one or more substituents independently selected from halogen, OH, C₁-C₄ alkyl, C₁-C₄ haloalkyl, C₁-C₄ alkoxy, benzyl, C₃-C₈ cycloalkyl, halophenoxy, 5- or 6-membered heteroaryl having one or more heteroatoms selected from N, O and S, and C₁-C₄ alkyl substituted by a 5- or 6-membered heterocycloalkyl having one or more heteroatoms selected from N, O and S;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, OH, C₁-C₄ alkyl, C₁-C₄ alkoxy, C₁-C₄ alkyl-NR^{1a}R^{1b}, and 5- or 6-membered heteroaryl having one or more heteroatoms selected from N, O and S;
   5- or 6-membered heteroaryl having one or more ring members selected from N, O, S and SO₂, substituted by one or more substituents independently selected from C₁-C₄ alkyl and C₁-C₄ haloalkyl;
   8- to 10-membered bicyclic heteroaryl having one or more ring members selected from N, O, S and SO₂, optionally substituted by one or more substitutents independently selected from halogen, C₁-C₄ alkyl and C₁-C₄ alkoxy; and
   phenoxy substituted by one or more C₁-C₄ alkyl;
R^{1a} and R^{1b} are each independently selected from H and C₁-C₄ alkyl;
R² is H;
R³ is selected from:
   H;
   CN;
   NR^{3a}R^{3b};
   C₁-C₄ alkyl optionally substituted by one or more substituents selected from OH and NR^{3c}R^{3d};
   C₃-C₈ cycloalkyl;
   3- to 8-membered heterocycloalkyl having one or more heteroatoms selected from N, O and S, optionally substituted by one or more C₁-C₄ alkyl;
   phenyl optionally substituted by one or more halogen;
   5- or 6-membered heteroaryl having one or more heteroatoms selected from N, O and S;
   C₁-C₄ alkoxy-C₁-C₄ alkyl;
   C₁-C₄ haloalkoxy-C₁-C₄ alkyl;
   C₃-C₈ cycloalkoxy-C₁-C₄ alkyl;
   C₃-C₈ cycloalkyl-C₁-C₄ alkoxy;
   C₁-C₄ alkyl-S—C₁-C₄ alkyl; and
   C₁-C₄ alkyl-SO₂-C₁-C₄ alkyl;
R^{3a} is selected from H, C₁-C₄ alkyl, and C₁-C₄ alkyl-NR^{3e}R^{3f};
R^{3b} is selected from H and C₁-C₄ alkyl;
R^{3c} is selected from H and C₁-C₄ alkyl;
R^{3d} is selected from C₁-C₄ alkyl;
R^{3e} and R^{3f} are each independently selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

Clause 8. The compound of any one of clauses 1 to 5, wherein
L1 is a bond, C₁-C₆ alkylene, C₁-C₆ haloalkylene, or C₃-C₆ cycloalkylene;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, 8- to 10-membered bicyclic heteroaryl having one or more ring members selected from N, O, S and SO₂, and phenoxy, wherein said alkyl, cycloalkyl, heterocycloalkyl, bicyclic cycloalkyl, bicyclic heterocycloalkyl, phenyl, heteroaryl, bicyclic heteroaryl, and phenoxy are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 5- or 6-membered heteroaryl having one or more heteroatoms selected from N, O and S, benzyl, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH and NR^{3c}R^{3d};
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

Clause 9. The compound of any one of clauses 1 to 6 or 8, wherein
L1 is a bond, C₁-C₆ alkylene, C₁-C₆ haloalkylene, or C₃-C₆ cycloalkylene;
R¹ is selected from:
   C₁-C₄ haloalkyl;
   C₃-C₈ cycloalkyl optionally substituted by one or more substituents independently selected from halogen, OH, CN, C₁-C₄ alkyl, C₁-C₄ alkoxy, and C₁-C₄ haloalkoxy;
   C₇-C₁₁ bicyclic cycloalkyl optionally substituted by one or more substituents independently selected from halogen, OH, C₁-C₄ alkyl, C₁-C₄ haloalkyl, C₁-C₄ alkoxy, benzyl, C₃-C₈ cycloalkyl, and C₁-C₄ alkyl substituted by a 5- or 6-membered heterocycloalkyl having one or more heteroatoms selected from N, O and S;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, OH, C₁-C₄ alkyl, C₁-C₄ alkoxy, C₁-C₄ alkyl-NR^{1a}R^{1b}, and 5- or 6-membered heteroaryl having one or more heteroatoms selected from N, O and S;
   5- or 6-membered heteroaryl having one or more ring members selected from N, O, S and SO₂, optionally substituted by one or more substituents independently selected from C₁-C₄ alkyl and C₁-C₄ haloalkyl;
   8- to 10-membered bicyclic heteroaryl having one or more ring members selected from N, O, S and SO₂, optionally substituted by one or more substituents independently selected halogen, C₁-C₄ alkyl and C₁-C₄ alkoxy; and
   phenoxy optionally substituted by one or more C₁-C₄ alkyl;
R^{1a} and R^{1b} are each independently selected from H and C₁-C₄ alkyl;
R² is H;
R³ is selected from:
   H;
   CN;
   NR^{3a}R^{3b};
   C₁-C₄ alkyl optionally substituted by one or more substituents independently selected from OH and NR^{3c}R^{3d};
   C₃-C₈ cycloalkyl;
   3- to 8-membered heterocycloalkyl having one or more heteroatoms selected from N, O and S;
   phenyl substituted by one or more halogen;
   5- or 6-membered heteroaryl having one or more heteroatoms selected from N, O and S;
   C₁-C₄ alkoxy-C₁-C₄ alkyl;
   C₃-C₈ cycloalkyl-C₁-C₄ alkoxy;
   C₃-C₈ cycloalkoxy-C₁-C₄ alkyl;
   C₁-C₄ haloalkoxy-C₁-C₄ alkyl;
   C₁-C₄ alkyl-S—C₁-C₄ alkyl; and
   C₁-C₄ alkyl-SO₂-C₁-C₄ alkyl;
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

Clause 10. The compound of preceding clause, wherein
L¹ is a bond, C₁-C₆ alkylene, C₁-C₆ haloalkylene, or C₃-C₆ cycloalkylene;
R¹ is selected from:
   C₁-C₄ haloalkyl;
   C₃-C₈ cycloalkyl optionally substituted by one or more substituents independently selected from halogen, OH, CN, C₁-C₄ alkyl, C₁-C₄ alkoxy, and C₁-C₄ haloalkoxy;
   C₇-C₁₁ bicyclic cycloalkyl optionally substituted by one or more substituents independently selected from halogen, OH, C₁-C₄ alkyl, C₁-C₄ haloalkyl, C₁-C₄ alkoxy, benzyl, C₃-C₈ cycloalkyl, and C₁-C₄ alkyl substituted by a 5- or 6-membered heterocycloalkyl having one or more heteroatoms selected from N, O and S;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, OH, C₁-C₄ alkyl, C₁-C₄ alkoxy, C₁-C₄ alkyl-NR^{1a}R^{1b}, and 5- or 6-membered heteroaryl having one or more heteroatoms selected from N, O and S;
   5- or 6-membered heteroaryl having one or more ring members selected from N, O, S and SO₂, substituted by one or more substituents independently selected from C₁-C₄ alkyl and C₁-C₄ haloalkyl;
   8- to 10-membered bicyclic heteroaryl having one or more ring members selected from N, O, S and SO₂, optionally substituted by one or more substituents independently selected from halogen, C₁-C₄ alkyl and C₁-C₄ alkoxy; and phenoxy substituted by one or more C₁-C₄ alkyl;
R^{1a} and R^{1b} are each independently selected from H and C₁-C₄ alkyl;
R² is H;
R³ is selected from:
   H;
   CN;
   NR^{3a}R^{3b};
   C₁-C₄ alkyl optionally substituted by one or more substituents independently selected from OH and NR^{3c}R^{3d};
   C₃-C₈ cycloalkyl;
   3- to 8-membered heterocycloalkyl having one or more heteroatoms selected from N, O and S;
   phenyl substituted by one or more halogen;
   5- or 6-membered heteroaryl having one or more heteroatoms selected from N, O and S;
   C₁-C₄ alkoxy-C₁-C₄ alkyl;
   C₃-C₈ cycloalkyl-C₁-C₄ alkoxy;
   C₃-C₈ cycloalkoxy-C₁-C₄ alkyl;
   C₁-C₄ haloalkoxy-C₁-C₄ alkyl;
   C₁-C₄ alkyl-S—C₁-C₄ alkyl; and
   C₁-C₄ alkyl-SO₂-C₁-C₄ alkyl;
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

Clause 11. The compound of any one of clauses 1 to 5 or 8, wherein
L¹ is a bond, C₁-C₆ alkylene, C₁-C₆ haloalkylene, or C₃-C₆ cycloalkylene;
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, and 5- or 6-membered heteroaryl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH and NR^{3c}R^{3d};
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from the following groups:

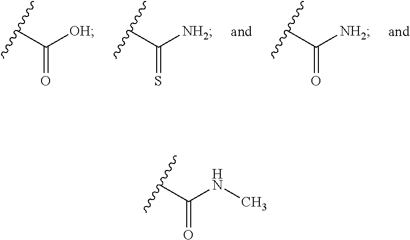

wherein [symbol] indicates the point of attachment to the rest of the compound;
R⁵ is selected from H and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

Clause 12. The compound of any one of clauses 1 to 5, 8, 9 or 11, wherein
L¹ is a bond, C₁-C₆ alkylene, C₁-C₆ haloalkylene, or C₃-C₆ cycloalkylene;
R¹ is selected from:
   C₃-C₈ cycloalkyl optionally substituted by one or more substituents independently selected from halogen, CN, C₁-C₄ alkyl, and C₁-C₄ haloalkoxy;
   C₇-C₁₁ bicyclic cycloalkyl optionally substituted by one or more substituents independently selected from halogen, OH, C₁-C₄ alkyl, C₁-C₄ haloalkyl, and C₁-C₄ alkoxy;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, OH, C₁-C₄ alkyl, C₁-C₄ alkoxy, C₁-C₄ alkyl-NR^{1a}R^{1b}, and 5- or 6-membered heteroaryl having one or more heteroatoms selected from N, O and S;
   5- or 6-membered heteroaryl having one or more ring members selected from N, O,
   S and SO₂, optionally substituted by one or more substituents independently selected from C₁-C₄ alkyl and C₁-C₄ haloalkyl; and
   8- to 10-membered bicyclic heteroaryl having one or more ring members selected from N, O, S and SO₂, optionally substituted substituted by one or more substituents independently selected from halogen and C₁-C₄ alkyl;
R^{1a} and R^{1b} are each independently selected from H and C₁-C₄ alkyl;
R² is H;
R³ is selected from:
   H;
   CN;
   NR^{3a}R^{3b};
   C₁-C₄ alkyl optionally substituted by one or more substituents independently selected from OH and NR^{3c}R^{3d};
   C₃-C₈ cycloalkyl;
   3- to 8-membered heterocycloalkyl having one or more heteroatoms selected from N, O and S;
   phenyl substituted by one or more halogen;
   5- or 6-membered heteroaryl having one or more heteroatoms selected from N, O and S;
   C₁-C₄ alkoxy-C₁-C₄ alkyl;
   C₃-C₈ cycloalkyl-C₁-C₄ alkoxy;
   C₃-C₈ cycloalkoxy-C₁-C₄ alkyl;
   C₁-C₄ haloalkoxy-C₁-C₄ alkyl;
   C₁-C₄ alkyl-S—C₁-C₄ alkyl; and
   C₁-C₄ alkyl-SO₂-C₁-C₄ alkyl;
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from the following groups:

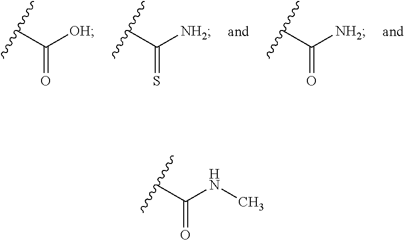

wherein [symbol] indicates the point of attachment to the rest of the compound;
R⁵ is selected from H and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

Clause 13. The compound of any preceding clause, wherein
L¹ is a bond, C₁-C₆ alkylene, C₁-C₆ haloalkylene, or C₃-C₆ cycloalkylene;
R¹ is selected from:
   C₃-C₈ cycloalkyl optionally substituted by one or more substituents independently selected from halogen, CN, C₁-C₄ alkyl, and C₁-C₄ haloalkoxy;
   C₇-C₁₁ bicyclic cycloalkyl optionally substituted by one or more substituents independently selected from halogen, OH, C₁-C₄ alkyl, C₁-C₄ haloalkyl, and C₁-C₄ alkoxy;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, OH, C₁-C₄ alkyl, C₁-C₄ alkoxy, C₁-C₄ alkyl-NR^{1a}R^{1b}, and 5- or 6-membered heteroaryl having one or more heteroatoms selected from N, O and S;
   5- or 6-membered heteroaryl having one or more ring members selected from N, O, S and SO₂, substituted by one or more substituents independently selected from C₁-C₄ alkyl and C₁-C₄ haloalkyl; and
   8- to 10-membered bicyclic heteroaryl having one or more ring members selected from N, O, S and SO₂, optionally substituted substituted by one or more substituents independently selected from halogen and C₁-C₄ alkyl;
R^{1a} and R^{1b} are each independently selected from H and C₁-C₄ alkyl;
R² is H;
R³ is selected from:
   H;
   CN;
   NR^{3a}R^{3b};
   C₁-C₄ alkyl optionally substituted by one or more substituents independently selected from OH and NR^{3c}R^{3d};
   C₃-C₈ cycloalkyl;
   3- to 8-membered heterocycloalkyl having one or more heteroatoms selected from N, O and S;
   phenyl substituted by one or more halogen;
   5- or 6-membered heteroaryl having one or more heteroatoms selected from N, O and S;
   C₁-C₄ alkoxy-C₁-C₄ alkyl;
   C₃-C₈ cycloalkyl-C₁-C₄ alkoxy;
   C₃-C₈ cycloalkoxy-C₁-C₄ alkyl;
   C₁-C₄ haloalkoxy-C₁-C₄ alkyl;
   C₁-C₄ alkyl-S—C₁-C₄ alkyl; and
   C₁-C₄ alkyl-SO₂-C₁-C₄ alkyl;
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from the following groups:

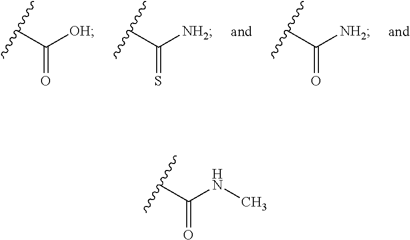

wherein [symbol] indicates the point of attachment to the rest of the compound;
R⁵ is selected from H, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

Clause 14. The compound of any one of clauses 1 to 5, 8 or 11, wherein
L¹ is a bond, C₁-C₆ alkylene, or C₃-C₆ cycloalkylene;
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, hetercycloalkyl, bicyclic cycloalkyl, bicyclic heterocycloalkyl, phenyl, heteroaryl, and bicyclic heteroaryl are each optionally substituted by one or more substituents independently selected from halogen, OH, C₁-C₄ alkyl, C₁-C₄ haloalkyl, C₁-C₄ alkoxy, and C₁-C₄ haloalkoxy;
R² is H;
R³ is selected from H, CN, NR^{3a}R^{3b}, C₁-C₄ alkyl optionally substituted by OH, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₃-C₈ cycloalkoxy-C₁-C₄ alkyl, C₃-C₈ cycloalkyl-C₁-C₄ alkoxy, C₁-C₄ haloalkoxy-C₁-C₄ alkyl, C₁-C₄ alkyl-S—C₁-C₄ alkyl, and C₁-C₄ alkyl-SO₂-C₁-C₄ alkyl;
R^{3a} is H;
R^{3b} is selected from C₁-C₄ alkyl;
R⁴ is selected from the following groups:

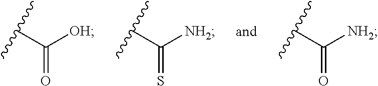

wherein [symbol] indicates the point of attachment to the rest of the compound;
R⁵ is H;
R⁶ is selected from H and C₁-C₄ alkyl; and
R⁷, R⁸, R⁹, R¹⁰, R¹¹, and R¹² are each H.

Clause 15. The compound of any one of clauses 1 to 5, 8, 9, 11, 12 or 14, wherein
L¹ is either a bond, C₁-C₆ alkylene, or C₃-C₆ cycloalkylene;
R¹ is selected from:
   C₃-C₈ cycloalkyl optionally substituted by one or more substituents independently selected from halogen, C₁-C₄ alkyl, and C₁-C₄ haloalkoxy;
   C₇-C₁₁ bicyclic cycloalkyl optionally substituted by one or more substituents independently selected from halogen, OH, C₁-C₄ alkyl, and C₁-C₄ alkoxy;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, C₁-C₄ alkyl and C₁-C₄ alkoxy;
   5- or 6-membered heteroaryl having one or more ring members selected from N, O, S and SO₂, optionally substituted by one or more substituents independently selected from C₁-C₄ alkyl and C₁-C₄ haloalkyl; and
   8- to 10-membered bicyclic heteroaryl having one or more ring members selected from N, O, S and SO₂, optionally substituted substituted by one or more substituents independently selected from halogen and C₁-C₄ alkyl;
R² is H;
R³ is selected from:
   H;
   CN;
   C₁-C₄ alkyl optionally substituted by OH;
   C₃-C₈ cycloalkyl;
   3- to 8-membered heterocycloalkyl having one or more heteroatoms selected from N, O and S;
   5- or 6-membered heteroaryl having one or more heteroatoms selected from N, O and S;
   C₁-C₄ alkoxy-C₁-C₄ alkyl;
   C₃-C₈ cycloalkyl-C₁-C₄ alkoxy;
   C₃-C₈ cycloalkoxy-C₁-C₄ alkyl
   C₁-C₄ haloalkoxy-C₁-C₄ alkyl;
   C₁-C₄ alkyl-S—C₁-C₄ alkyl; and
   C₁-C₄ alkyl-SO₂-C₁-C₄ alkyl;
R^{3a} is H;
R^{3b} is selected from C₁-C₄ alkyl.
R⁴ is selected from the following groups:

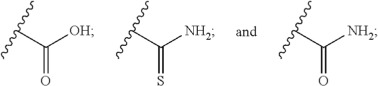

wherein [symbol] indicates the point of attachment to the rest of the compound;
R⁵ is H;
R⁶ is selected from H and C₁-C₄ alkyl; and
R⁷, R⁸, R⁹, R¹⁰, R¹¹, and R¹² are each H.

Clause 16. The compound of any preceding clause, wherein
L¹ is either a bond, C₁-C₆ alkylene, or C₃-C₆ cycloalkylene;
R¹ is selected from:
   C₃-C₈ cycloalkyl optionally substituted by one or more substituents independently selected from halogen, C₁-C₄ alkyl, and C₁-C₄ haloalkoxy;
   C₇-C₁₁ bicyclic cycloalkyl optionally substituted by one or more substituents independently selected from halogen, OH, C₁-C₄ alkyl, and C₁-C₄ alkoxy;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, C₁-C₄ alkyl and C₁-C₄ alkoxy;
   5- or 6-membered heteroaryl having one or more ring members selected from N, O, S and SO₂, substituted by one or more substituents independently selected from C₁-C₄ alkyl and C₁-C₄ haloalkyl; and
   8- to 10-membered bicyclic heteroaryl having one or more ring members selected from N, O, S and SO₂, optionally substituted substituted by one or more substituents independently selected from halogen and C₁-C₄ alkyl;
R² is H;
R³ is selected from:
   H;
   CN;
   NR^{3a}R^{3b};
   C₁-C₄ alkyl optionally substituted by OH;
   C₃-C₈ cycloalkyl;
   3- to 8-membered heterocycloalkyl having one or more heteroatoms selected from N, O and S;
   5- or 6-membered heteroaryl having one or more heteroatoms selected from N, O and S;
   C₁-C₄ alkoxy-C₁-C₄ alkyl;
   C₃-C₈ cycloalkyl-C₁-C₄ alkoxy;
   C₃-C₈ cycloalkoxy-C₁-C₄ alkyl;
   C₁-C₄ haloalkoxy-C₁-C₄ alkyl;
   C₁-C₄ alkyl-S—C₁-C₄ alkyl; and
   C₁-C₄ alkyl-SO₂-C₁-C₄ alkyl;
R^{3a} is H;
R^{3b} is selected from C₁-C₄ alkyl.
R⁴ is selected from the following groups:

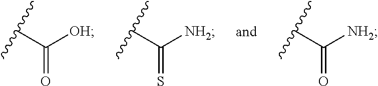

wherein [symbol] indicates the point of attachment to the rest of the compound;
R⁵ is H;
R⁶ is selected from H and C₁-C₄ alkyl; and
R⁷, R⁸, R⁹, R¹⁰, R¹¹, and R¹² are each H.

Clause 17. The compound of any one of clauses 1 to 5, 8, 11 or 14, wherein
L¹ is either a bond or C₁-C₆ alkylene;
R¹ is selected from C₃-C₈ cycloalkyl, C₈-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, OH, C₁-C₄ alkyl, C₁-C₄ haloalkyl, C₁-C₄ alkoxy, and C₁-C₄ haloalkoxy;
R² is H;
R³ is selected from NR^{3a}R^{3b}, C₁-C₄ alkyl optionally substituted by one or more substituents independently selected from OH and NR^{3c}R^{3d}, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₃-C₈ cycloalkoxy-C₁-C₄ alkyl, C₁-C₄ alkoxy-C₁-C₄ alkyl, C₁-C₄ haloalkoxy-C₁-C₄ alkyl, C₃-C₈ cycloalkyl-C₁-C₄ alkoxy, and alkyl-S—C₁-C₄ alkyl;
R^{3a} is H;
R^{3b} is selected from C₁-C₄ alkyl;
R⁴ is selected from the following groups:

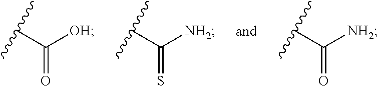

wherein [symbol] indicates the point of attachment to the rest of the compound;
R⁵, R⁷, R⁸, R⁹, R¹⁰, R¹¹, and R¹² are each H; and
R⁶ is selected from H and C₁-C₄ alkyl.

Clause 18. The compound of any one of clauses 1 to 5, 8, 9, 11, 12, 14, 15 or 17, wherein
L¹ is either a bond or C₁-C₆ alkylene;
R¹ is selected from:
   C₃-C₈ cycloalkyl optionally substituted by one or more substituents independently selected from halogen and C₁-C₄ haloalkoxy;
   C₈-C₁₁ bicyclic cycloalkyl optionally substituted by one or more substituents independently selected from halogen, OH, C₁-C₄ alkyl and C₁-C₄ alkoxy;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen and C₁-C₄ alkyl;
   5- or 6-membered heteroaryl having one or more ring members selected from N, O, S and SO₂, optionally substituted by one or more substituents independently selected from C₁-C₄ alkyl and C₁-C₄ haloalkyl; and
   8- to 10-membered bicyclic heteroaryl having one or more ring members selected from N, O, S and SO₂, optionally substituted substituted by one or more substituents independently selected from halogen and C₁-C₄ alkyl;
R² is H;
R³ is selected from:
   NR^{3a}R^{3b}.
   C₁-C₄ alkyl optionally substituted by OH;
   C₃-C₈ cycloalkyl;
   3- to 8-membered heterocycloalkyl having one or more heteroatoms selected from N, O and S;
   5- or 6-membered heteroaryl having one or more heteroatoms selected from N, O and S;
   C₁-C₄ alkoxy-C₁-C₄ alkyl;
   C₃-C₈ cycloalkyl-C₁-C₄ alkoxy;
   C₃-C₈ cycloalkoxy-C₁-C₄ alkyl;
   C₁-C₄ haloalkoxy-C₁-C₄ alkyl; and
   C₁-C₄ alkyl-S—C₁-C₄ alkyl;
R^{3a} is H;
R^{3b} is selected from C₁-C₄ alkyl;
R⁴ is selected from the following groups:

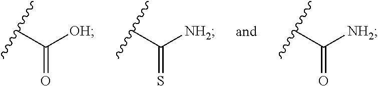

wherein [symbol] indicates the point of attachment to the rest of the compound H;
R⁵, R⁷, R⁸, R⁹, R¹⁰, R¹¹, and R¹² are each H; and
R⁶ is selected from H and C₁-C₄ alkyl.

Clause 19. The compound of any preceding clause, wherein
L¹ is either a bond or C₁-C₆ alkylene;
R¹ is selected from:
   C₃-C₈ cycloalkyl substituted by one or more substituents independently selected from halogen and C₁-C₄ haloalkoxy;
   C₈-C₁₁ bicyclic cycloalkyl optionally substituted by one or more substituents independently selected from halogen, OH, C₁-C₄ alkyl and C₁-C₄ alkoxy;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl substituted by one or more substituents independently selected from halogen and C₁-C₄ alkyl;
   5- or 6-membered heteroaryl having one or more ring members selected from N, O, S and SO₂, substituted by one or more substituents independently selected from C₁-C₄ alkyl and C₁-C₄ haloalkyl; and
   8- to 10-membered bicyclic heteroaryl having one or more ring members selected from N, O, S and SO₂, substituted by one or more substituents independently selected from halogen and C₁-C₄ alkyl;
R² is H;
R³ is selected from:
   NR^{3a}R^{3b};
   C₁-C₄ alkyl optionally substituted by OH;
   C₃-C₈ cycloalkyl;
   3- to 8-membered heterocycloalkyl having one or more heteroatoms selected from N, O and S;
   5- or 6-membered heteroaryl having one or more heteroatoms selected from N, O and S;
   C₁-C₄ alkoxy-C₁-C₄ alkyl;
   C₃-C₈ cycloalkyl-C₁-C₄ alkoxy;
   C₃-C₈ cycloalkoxy-C₁-C₄ alkyl;
   C₁-C₄ haloalkoxy-C₁-C₄ alkyl; and
   C₁-C₄ alkyl-S—C₁-C₄ alkyl;
R^{3a} is H;
R^{3b} is selected from C₁-C₄ alkyl;
R⁴ is selected from the following groups:

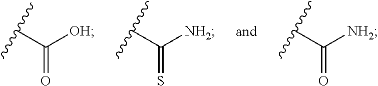

wherein [symbol] indicates the point of attachment to the rest of the compound;
R⁵, R⁷, R⁸, R⁹, R¹⁰, R¹¹, and R¹² are each H; and
R⁶ is selected from H and C₁-C₄ alkyl.

Clause 20. The compound of any one of clauses 1 to 19, wherein Q is O.

Clause 21. The compound of any one of clauses 1 to 7 and 20, wherein L1 is a bond, C₁-C₆ alkylene, C₁-C₆ haloalkylene, C₃-C₆ cycloalkylene, or a 5- or 6-membered heterocycloalkylene having one or more heteroatoms selected from N, O and S.

Clause 22. The compound of any one of clauses 1 to 7, 20 and 21, wherein L1 is a bond, C₁-C₆ alkylene, C₁-C₆ haloalkylene, or C₃-C₆ cycloalkylene.

Clause 23. The compound of any one of clauses 1 to 7, and 20 to 22, wherein L1 is a bond, methylene, ethylidene, 2,2,2-trifluoroethylidene, and cyclopropylidene.

Clause 24. The compound of any one of clauses 1 to 7, 20 and 21, wherein L1 is a bond, C₁-C₆ alkylene or a 5- or 6-membered heterocycloalkyl having one or more heteroatoms selected from N, O and S.

Clause 25. The compound of any one of clauses 1 to 22 and 24, wherein L1 is either a bond or C₁-C₆ alkylene.

Clause 26. The compound of any preceding clause, wherein L¹ is either a bond or methylene.

Clause 27. The compound of any one of clauses 1 to 22, 24 and 25, wherein L1 is C₁-C₆ alkylene.

Clause 28. The compound of any preceding clause, wherein L1 is methylene.

Clause 29. The compound of any one of clauses 1 to 26, wherein L1 is a bond.

Clause 30. The compound of any one of clauses 1 to 7, 20 and 21, wherein L1 is a 5- or 6-membered heterocycloalkylene having one or more heteroatoms selected from N, O and S.

Clause 31. The compound of any one of clauses 1 to 3 and 20 to 30, wherein
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, C₉-C₁₀ bicyclic aryl, 5- or 6-membered heteroaryl having one or more ring members selected from N, O, S and SO₂, 8- to 10-membered bicyclic heteroaryl having one or more ring members selected from N, O, S and SO₂, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S; and
R^{1a} and R^{1b} are each independently selected from H and C₁-C₄ alkyl.

Clause 32. The compound of any one of clauses 1 to 3, or 20 to 31, wherein
R¹ is selected from C₁-C₄ alkyl, C₃-C₈ cycloalkyl, C₈-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, C₉-C₁₀ bicyclic aryl, 5- or 6-membered heteroaryl having one or more ring members selected from N, O, S and SO₂, 8- to 10-membered bicyclic heteroaryl having one or more ring members selected from N, O, S and SO₂, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, C₉-C₁₀ bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S; and
R^{1a} and R^{1b} are each independently selected from H and C₁-C₄ alkyl.

Clause 33. The compound of any one of clauses 1 to 3 or 20 to 32, wherein
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, 8- to 10-membered bicyclic heteroaryl having one or more ring members selected from N, O, S and SO₂, and phenoxy, wherein said alkyl, cycloalkyl, heterocycloalkyl, bicyclic cycloalkyl, bicyclic heterocycloalkyl, phenyl, heteroaryl, bicyclic heteroaryl, and phenoxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S; and
R^{1a} and R^{1b} are each independently selected from H and C₁-C₄ alkyl.

Clause 34. The compound of any one of clauses 1 to 3 or 20 to 33, wherein
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, heterocycloalkyl, bicyclic cycloalkyl, bicyclic heterocycloalkyl, phenyl, heteroaryl, and bicyclic heteroaryl are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S; and
R^{1a} and R^{1b} are each independently selected from H and C₁-C₄ alkyl.

Clause 35. The compound of any one of clauses 1 to 3 or 20 to 34, wherein
R¹ is selected from C₃-C₈ cycloalkyl, C₈-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S; and
R^{1a} and R^{1b} are each independently selected from H and C₁-C₄ alkyl.

Clause 36. The compound of any one of clauses 1 to 5 or 20 to 35, wherein
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, 8- to 10-membered bicyclic heteroaryl having one or more ring members selected from N, O, S and SO₂, and phenoxy, wherein said alkyl, cycloalkyl, heterocycloalkyl, bicyclic cycloalkyl, bicyclic heterocycloalkyl, phenyl, heteroaryl, bicyclic heteroaryl, and phenoxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, 5- or 6-membered heteroaryl having one or more heteroatoms selected from N, O and S, benzyl, halophenoxy, and C₁-C₄ alkyl substituted by a 5- or 6-membered heterocycloalkyl having one or more heteroatoms selected from N, O and S; and
R^{1a} and R^{1b} are each independently selected from H and C₁-C₄ alkyl.

Clause 37. The compound of any one of clauses 1 to 5, 20 to 34 or 36, wherein
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, 5- or 6-membered heteroaryl having one or more heteroatoms selected from N, O and S, benzyl, halophenoxy, and C₁-C₄ alkyl substituted by a 5- or 6-membered heterocycloalkyl having one or more heteroatoms selected from N, O and S; and
R^{1a} and R^{1b} are each independently selected from H and C₁-C₄ alkyl.

Clause 38. The compound of any one of clauses 1 to 5 or 20 to 37, wherein
R¹ is selected from C₃-C₈ cycloalkyl, C₈-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, 5- or 6-membered heteroaryl having one or more heteroatoms selected from N, O and S, benzyl, halophenoxy, and C₁-C₄ alkyl substituted by a 5- or 6-membered heterocycloalkyl having one or more heteroatoms selected from N, O and S; and
R^{1a} and R^{1b} are each independently selected from H and C₁-C₄ alkyl.

Clause 39. The compound of any one of clauses 1 to 5, 8, 20 to 33, or 36, wherein
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, 8- to 10-membered bicyclic heteroaryl having one or more ring members selected from N, O, S and SO₂, and phenoxy, wherein said alkyl, cycloalkyl, heterocycloalkyl, bicyclic cycloalkyl, bicyclic heterocycloalkyl, phenyl, heteroaryl, bicyclic heteroaryl, and phenoxy are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 5- or 6-membered heteroaryl having one or more heteroatoms selected from N, O and S, benzyl, and C₁-C₄ alkyl substituted by a 5- or 6-membered heterocycloalkyl having one or more heteroatoms selected from N, O and S; and R^{1a} and R^{1b} are each independently selected from H and C₁-C₄ alkyl.

Clause 40. The compound of any one of clauses 1 to 5, 8, 20 to 33, 36, 37 or 39, wherein
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 5- or 6-membered heteroaryl having one or more heteroatoms selected from N, O and S, benzyl, and C₁-C₄ alkyl substituted by a 5- or 6-membered heterocycloalkyl having one or more heteroatoms selected from N, O and S; and
R^{1a} and R^{1b} are each independently selected from H and C₁-C₄ alkyl.

Clause 41. The compound of any one of clauses 1 to 5, 8 or 20 to 40, wherein
R¹ is selected from C₃-C₈ cycloalkyl, C₈-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 5- or 6-membered heteroaryl having one or more heteroatoms selected from N, O and S, benzyl, and C₁-C₄ alkyl substituted by a 5- or 6-membered heterocycloalkyl having one or more heteroatoms selected from N, O and S; and
R^{1a} and R^{1b} are each independently selected from H and C₁-C₄ alkyl.

Clause 42. The compound of any one of clauses 1 to 5, 8, 11, 20 to 34, 36, 37, 39 or 40, wherein
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, and 5- or 6-membered heteroaryl having one or more heteroatoms selected from N, O and S; and
R^{1a} and R^{1b} are each independently selected from H and C₁-C₄ alkyl.

Clause 43. The compound of any one of clause 1 to 5, 8, 11 or 20 to 42, wherein
R¹ is selected from C₃-C₈ cycloalkyl, C₈-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, and 5- or 6-membered heteroaryl having one or more heteroatoms selected from N, O and S; and
R^{1a} and R^{1b} are each independently selected from H and C₁-C₄ alkyl.

Clause 44. The compound of any one of clauses 1 to 5, 8, 11, 14, 20 to 34, 36, 37, 39, 40 or 42, wherein
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, heterocycloalkyl, bicyclic cycloalkyl, bicyclic heterocycloalkyl, phenyl, heteroaryl, and bicyclic heteroaryl are each optionally substituted by one or more substituents independently selected from halogen, OH, C₁-C₄ alkyl, C₁-C₄ haloalkyl, C₁-C₄ alkoxy, and C₁-C₄ haloalkoxy.

Clause 45. The compound of any one of clauses 1 to 5, 8, 11, 14 or 20 to 44, wherein
R¹ is selected from C₃-C₈ cycloalkyl, C₈-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, heterocycloalkyl, bicyclic cycloalkyl, bicyclic heterocycloalkyl, phenyl, heteroaryl, and bicyclic heteroaryl are each optionally substituted by one or more substituents independently selected from halogen, OH, C₁-C₄ alkyl, C₁-C₄ haloalkyl, C₁-C₄ alkoxy, and C₁-C₄ haloalkoxy.

Clause 46. The compound of any one of clauses 1 to 6, 20 to 33 or 36, wherein
R¹ is selected from:
   C₁-C₄ haloalkyl;
   C₃-C₈ cycloalkyl optionally substituted by one or more substituents independently selected from halogen, OH, CN, NR^{1a}R^{1b}, C₁-C₄ alkyl, C₁-C₄ alkoxy, and C₁-C₄ haloalkoxy;
   C₇-C₁₁ bicyclic cycloalkyl optionally substituted by one or more substituents independently selected from halogen, OH, C₁-C₄ alkyl, C₁-C₄ haloalkyl, C₁-C₄ alkoxy, benzyl, C₃-C₈ cycloalkyl, halophenoxy, 5- or 6-membered heteroaryl having one or more heteroatoms selected from N, O and S, and C₁-C₄ alkyl substituted by a 5- or 6-membered heterocycloalkyl having one or more heteroatoms selected from N, O and S;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, OH, C₁-C₄ alkyl, C₁-C₄ alkoxy, C₁-C₄ alkyl-NR^{1a}R^{1b}, and 5- or 6-membered heteroaryl having one or more heteroatoms selected from N, O and S;
   5- or 6-membered heteroaryl having one or more ring members selected from N, O, S and SO₂, optionally substituted by one or more substituents independently selected from C₁-C₄ alkyl and C₁-C₄ haloalkyl;
   8- to 10-membered bicyclic heteroaryl having one or more ring members selected from N, O, S and SO₂, optionally substituted by one or more substitutents independently selected from halogen, C₁-C₄ alkyl and C₁-C₄ alkoxy; and
   phenoxy optionally substituted by one or more C₁-C₄ alkyl;
wherein R^{1a} and R^{1b} are each independently selected from H and C₁-C₄ alkyl.

Clause 47. The compound of any one of clauses 1 to 7, 20 to 33, 36 or 46, wherein
R¹ is selected from:
   C₁-C₄ haloalkyl;
   C₃-C₈ cycloalkyl optionally substituted by one or more substituents independently selected from halogen, OH, CN, NR^{1a}R^{1b}, C₁-C₄ alkyl, C₁-C₄ alkoxy, and C₁-C₄ haloalkoxy;
   C₇-C₁₁ bicyclic cycloalkyl optionally substituted by one or more substituents independently selected from halogen, OH, C₁-C₄ alkyl, C₁-C₄ haloalkyl, C₁-C₄ alkoxy, benzyl, C₃-C₈ cycloalkyl, halophenoxy, 5- or 6-membered heteroaryl having one or more heteroatoms selected from N, O and S, and C₁-C₄ alkyl substituted by a 5- or 6-membered heterocycloalkyl having one or more heteroatoms selected from N, O and S;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, OH, C₁-C₄ alkyl, C₁-C₄ alkoxy, C₁-C₄ alkyl-NR^{1a}R^{1b}, and 5- or 6-membered heteroaryl having one or more ring members selected from N, O, S and SO₂ (e.g having one or more ring members selected from N, O and S);
   5- or 6-membered heteroaryl having one or more ring members selected from N, O, S and SO₂, substituted by one or more substituents independently selected from C₁-C₄ alkyl and C₁-C₄ haloalkyl;
   8- to 10-membered bicyclic heteroaryl having one or more ring members selected from N, O, S and SO₂, optionally substituted by one or more substitutents independently selected from halogen, C₁-C₄ alkyl and C₁-C₄ alkoxy; and phenoxy substituted by one or more C₁-C₄ alkyl;
wherein R^{1a} and R^{1b} are each independently selected from H and C₁-C₄ alkyl.

Clause 48. The compound of any one of clauses 1 to 6, 8, 9, 20 to 33, 36, 39 or 46 wherein
R¹ is selected from:
   C₁-C₄ haloalkyl;
   C₃-C₈ cycloalkyl optionally substituted by one or more substituents independently selected from halogen, OH, CN, C₁-C₄ alkyl, C₁-C₄ alkoxy, and C₁-C₄ haloalkoxy;
   C₇-C₁₁ bicyclic cycloalkyl optionally substituted by one or more substituents independently selected from halogen, OH, C₁-C₄ alkyl, C₁-C₄ haloalkyl, C₁-C₄ alkoxy, benzyl, C₃-C₈ cycloalkyl, and C₁-C₄ alkyl substituted by a 5- or 6-membered heterocycloalkyl having one or more heteroatoms selected from N, O and S;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, OH, C₁-C₄ alkyl, C₁-C₄ alkoxy, C₁-C₄ alkyl-NR^{1a}R^{1b}, and 5- or 6-membered heteroaryl having one or more heteroatoms selected from N, O and S;
   5- or 6-membered heteroaryl having one or more ring members selected from N, O, S and SO₂, optionally substituted by one or more substituents independently selected from C₁-C₄ alkyl and C₁-C₄ haloalkyl;
   8- to 10-membered bicyclic heteroaryl having one or more ring members selected from N, O, S and SO₂, optionally substituted by one or more substituents independently selected from halogen, C₁-C₄ alkyl and C₁-C₄ alkoxy; and phenoxy optionally substituted by one or more C₁-C₄ alkyl;
wherein R^{1a} and R^{1b} are each independently selected from H and C₁-C₄ alkyl.

Clause 49. The compound of any one of clauses 1 to 10, 20 to 33, 36, 39, 46 and 47, wherein
R¹ is selected from:
   C₁-C₄ haloalkyl;
   C₃-C₈ cycloalkyl optionally substituted by one or more substituents independently selected from halogen, OH, CN, C₁-C₄ alkyl, C₁-C₄ alkoxy, and C₁-C₄ haloalkoxy;
   C₇-C₁₁ bicyclic cycloalkyl optionally substituted by one or more substituents independently selected from halogen, OH, C₁-C₄ alkyl, C₁-C₄ haloalkyl, C₁-C₄ alkoxy, benzyl, C₃-C₈ cycloalkyl, and C₁-C₄ alkyl substituted by a 5- or 6-membered heterocycloalkyl having one or more heteroatoms selected from N, O and S;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, OH, C₁-C₄ alkyl, C₁-C₄ alkoxy, C₁-C₄ alkyl-NR^{1a}R^{1b}, and 5- or 6-membered heteroaryl having one or more heteroatoms selected from N, O and S;
   5- or 6-membered heteroaryl having one or more ring members selected from N, O, S and SO₂, substituted by one or more substituents independently selected from C₁-C₄ alkyl and C₁-C₄ haloalkyl;
   8- to 10-membered bicyclic heteroaryl having one or more ring members selected from N, O, S and SO₂, optionally substituted by one or more substituents independently selected from halogen, C₁-C₄ alkyl and C₁-C₄ alkoxy; and
   phenoxy substituted by one or more C₁-C₄ alkyl;
wherein R^{1a} and R^{1b} are each independently selected from H and C₁-C₄ alkyl.

Clause 50. The compound of any one of clauses 1 to 6, 8, 9, 11, 12, 20 to 34, 36, 37, 39, 40, 42, 46 or 48, wherein
R¹ is selected from:
   C₃-C₈ cycloalkyl optionally substituted by one or more substituents independently selected from halogen, CN, C₁-C₄ alkyl, and C₁-C₄ haloalkoxy;
   C₇-C₁₁ bicyclic cycloalkyl optionally substituted by one or more substituents independently selected from halogen, OH, C₁-C₄ alkyl, C₁-C₄ haloalkyl, and C₁-C₄ alkoxy;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, OH, C₁-C₄ alkyl, C₁-C₄ alkoxy, C₁-C₄ alkyl-NR^{1a}R^{1b}, and 5- or 6-membered heteroaryl having one or more heteroatoms selected from N, O and S;
   5- or 6-membered heteroaryl having one or more ring members selected from N, O, S and SO₂, optionally substituted by one or more substituents independently selected from C₁-C₄ alkyl and C₁-C₄ haloalkyl; and
   8- to 10-membered bicyclic heteroaryl having one or more ring members selected from N, O, S and SO₂, optionally substituted substituted by one or more substituents independently selected from halogen and C₁-C₄ alkyl;
wherein R^{1a} and R^{1b} are each independently selected from H and C₁-C₄ alkyl.

Clause 51. The compound of any one of clauses 1 to 13, 20 to 34, 36, 37, 39, 40, 42 or 46 to 50, wherein
R¹ is selected from:
   C₃-C₈ cycloalkyl optionally substituted by one or more substituents independently selected from halogen, CN, C₁-C₄ alkyl, and C₁-C₄ haloalkoxy;
   C₇-C₁₁ bicyclic cycloalkyl optionally substituted by one or more substituents independently selected from halogen, OH, C₁-C₄ alkyl, C₁-C₄ haloalkyl, and C₁-C₄ alkoxy;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, OH, C₁-C₄ alkyl, C₁-C₄ alkoxy, C₁-C₄ alkyl-NR^{1a}R^{1b}, and 5- or 6-membered heteroaryl having one or more heteroatoms selected from N, O and S;
   5- or 6-membered heteroaryl having one or more ring members selected from N, O, S and SO₂, substituted by one or more substituents independently selected from C₁-C₄ alkyl and C₁-C₄ haloalkyl; and
   8- to 10-membered bicyclic heteroaryl having one or more ring members selected from N, O, S and SO₂, optionally substituted substituted by one or more substituents independently selected from halogen and C₁-C₄ alkyl;
wherein R^{1a} and R^{1b} are each independently selected from H and C₁-C₄ alkyl.

Clause 52. The compound of any one of clauses 1 to 6, 8, 9, 11, 12, 14, 15, 20 to 34, 36, 37, 39, 40, 42, 44, 46, 48 or 50, wherein
R¹ is selected from:
   C₃-C₈ cycloalkyl optionally substituted by one or more substituents independently selected from halogen, C₁-C₄ alkyl, and C₁-C₄ haloalkoxy;
   C₇-C₁₁ bicyclic cycloalkyl optionally substituted by one or more substituents independently selected from halogen, OH, C₁-C₄ alkyl, and C₁-C₄ alkoxy;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, C₁-C₄ alkyl and C₁-C₄ alkoxy;
   5- or 6-membered heteroaryl having one or more ring members selected from N, O, S and SO₂, optionally substituted by one or more substituents independently selected from C₁-C₄ alkyl and C₁-C₄ haloalkyl; and
   8- to 10-membered bicyclic heteroaryl having one or more ring members selected from N, O, S and SO₂, optionally substituted substituted by one or more substituents independently selected from halogen and C₁-C₄ alkyl.

Clause 53. The compound of any of clauses 1 to 15, 20 to 34, 36, 37, 39, 40, 42, 44 or 46 to 52, wherein
R¹ is selected from:
   C₃-C₈ cycloalkyl optionally substituted by one or more substituents independently selected from halogen, C₁-C₄ alkyl, and C₁-C₄ haloalkoxy;
   C₇-C₁₁ bicyclic cycloalkyl optionally substituted by one or more substituents independently selected from halogen, OH, C₁-C₄ alkyl, and C₁-C₄ alkoxy;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen, C₁-C₄ alkyl and C₁-C₄ alkoxy;
   5- or 6-membered heteroaryl having one or more ring members selected from N, O, S and SO₂, substituted by one or more substituents independently selected from C₁-C₄ alkyl and C₁-C₄ haloalkyl; and
   8- to 10-membered bicyclic heteroaryl having one or more ring members selected from N, O, S and SO₂, optionally substituted substituted by one or more substituents independently selected from halogen and C₁-C₄ alkyl.

Clause 54. The compound of any one of clauses 1 to 6, 8, 9, 11, 12, 14, 15, 17, 18, 20 to 46, 48, 50 or 52, wherein
R¹ is selected from:
   C₃-C₈ cycloalkyl optionally substituted by one or more substituents independently selected from halogen and C₁-C₄ haloalkoxy;
   C₈-C₁₁ bicyclic cycloalkyl optionally substituted by one or more substituents independently selected from halogen, OH, C₁-C₄ alkyl and C₁-C₄ alkoxy;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl optionally substituted by one or more substituents independently selected from halogen and C₁-C₄ alkyl;
   5- or 6-membered heteroaryl having one or more ring members selected from N, O, S and SO₂, optionally substituted by one or more substituents independently selected from C₁-C₄ alkyl and C₁-C₄ haloalkyl; and
   8- to 10-membered bicyclic heteroaryl having one or more ring members selected from N, O, S and SO₂, optionally substituted substituted by one or more substituents independently selected from halogen and C₁-C₄ alkyl.

Clause 55. The compound of any of preceding clause, wherein
R¹ is selected from:
   C₃-C₈ cycloalkyl substituted by one or more substituents independently selected from halogen and C₁-C₄ haloalkoxy;
   C₈-C₁₁ bicyclic cycloalkyl optionally substituted by one or more substituents independently selected from halogen, OH, C₁-C₄ alkyl and C₁-C₄ alkoxy;
   3- to 8-membered heterocycloalkyl having one or more ring members selected from N, O, S and SO₂;
   8- to 11-membered bicyclic heterocycloalkyl having one or more ring members selected from N, O, S and SO₂, optionally substituted by one or more substituents independently selected from halogen;
   phenyl substituted by one or more substituents independently selected from halogen and C₁-C₄ alkyl;
   5- or 6-membered heteroaryl having one or more ring members selected from N, O, S and SO₂, substituted by one or more substituents independently selected from C₁-C₄ alkyl and C₁-C₄ haloalkyl; and
   8- to 10-membered bicyclic heteroaryl having one or more ring members selected from N, O, S and SO₂, substituted by one or more substituents independently selected from halogen and C₁-C₄ alkyl.

Clause 56. The compound of any one of clauses 1 to 7, 20 to 33, 36, 46 or 47, wherein
R¹ is selected from the following groups:

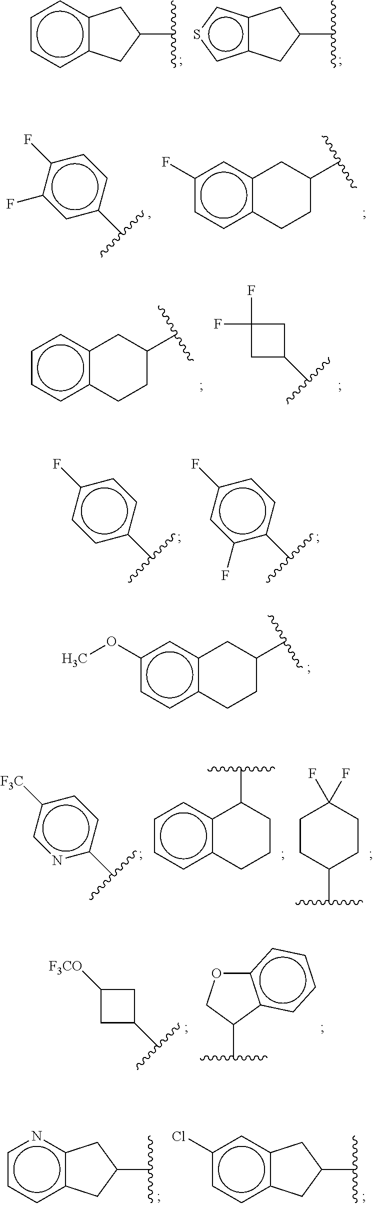

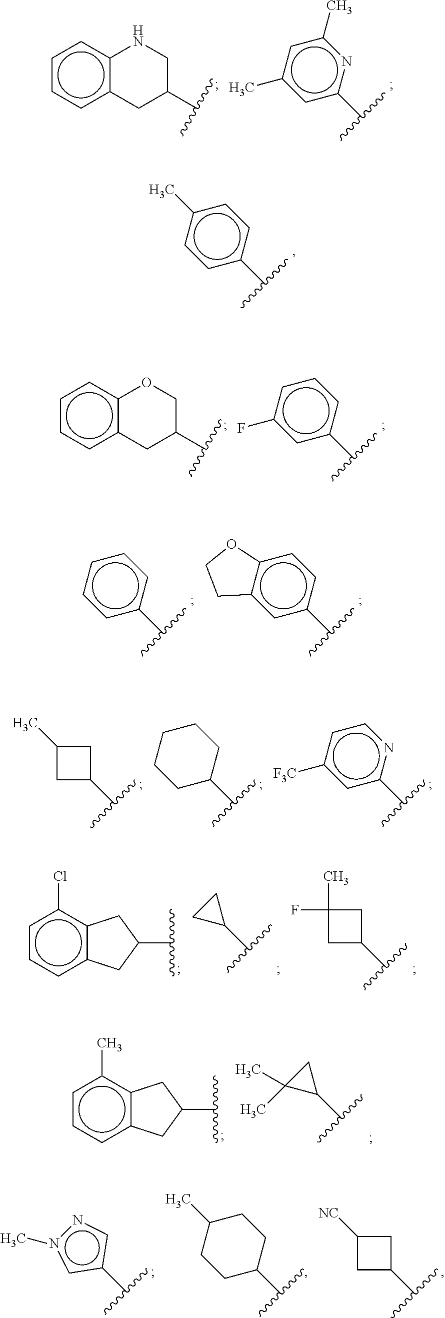

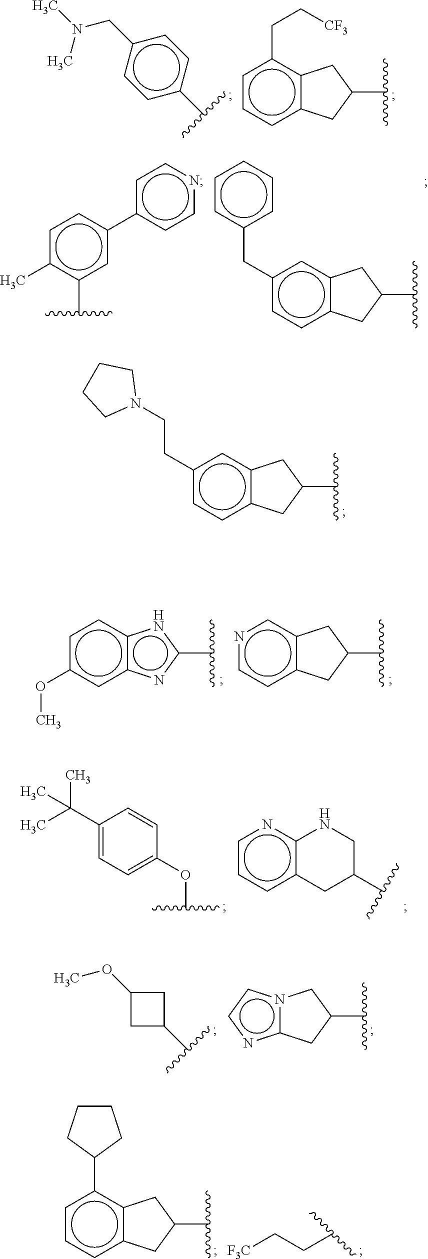

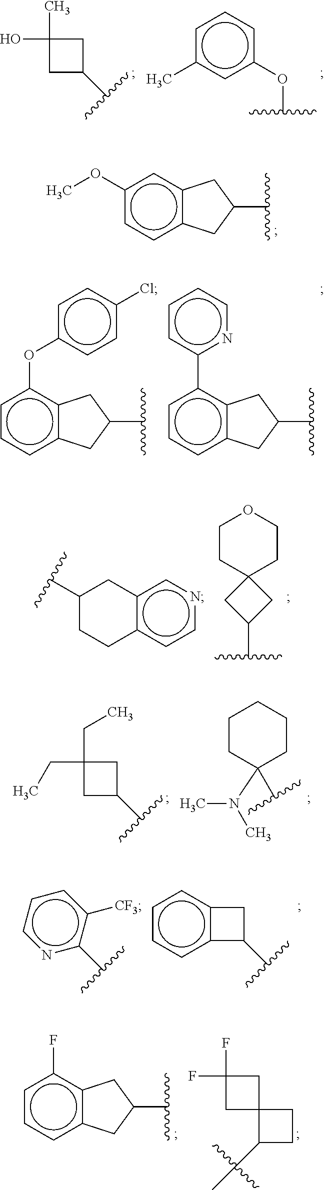

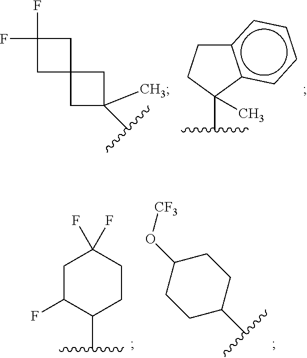

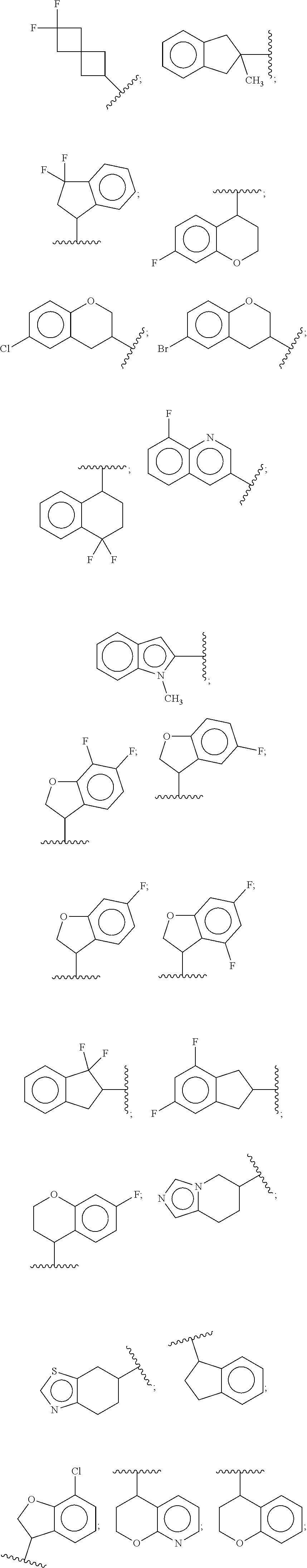

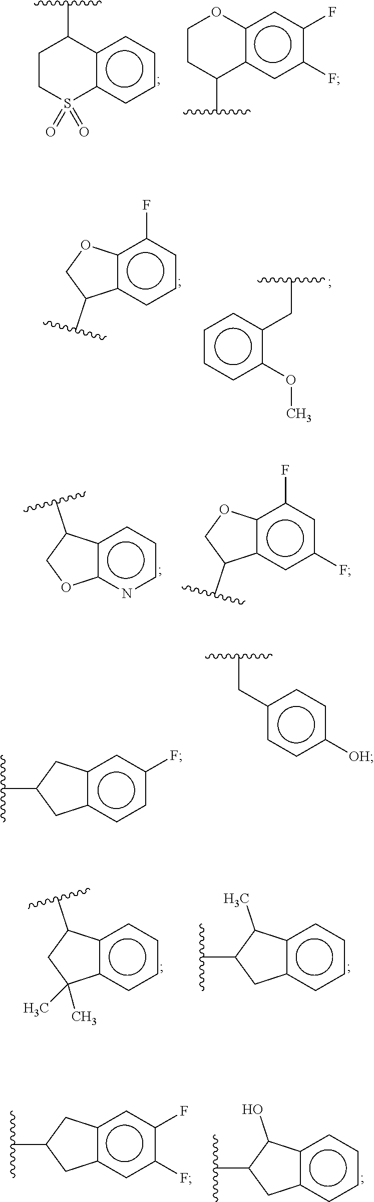

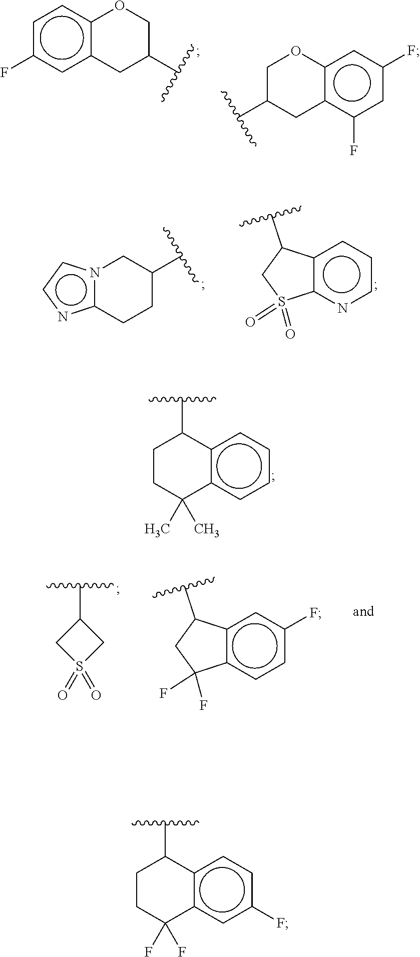

wherein [symbol] indicates the point of attachment to the rest of the compound.

Clause 57. The compound of any one of clauses 1 to 7, 20 to 33, 36, 46, 47 or 52, wherein
R¹ is selected from the following groups:

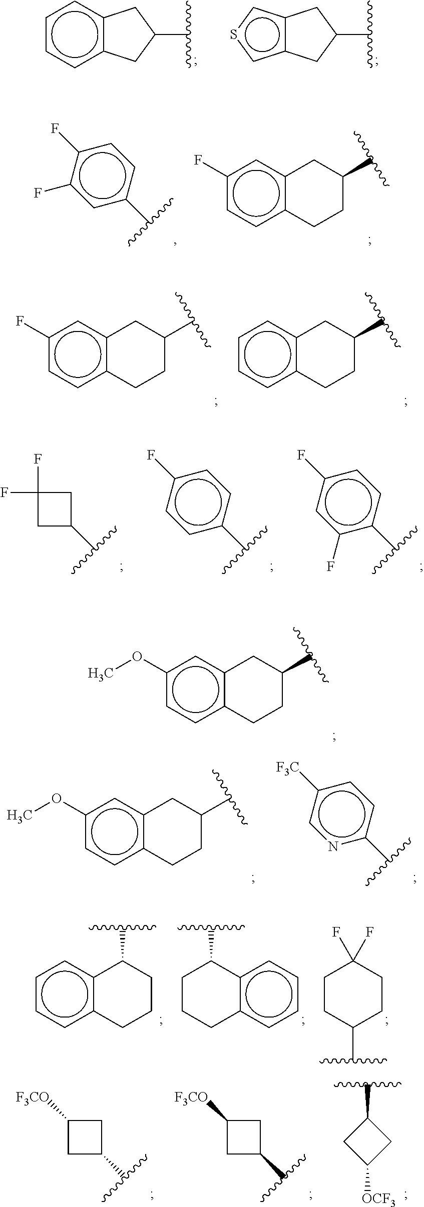

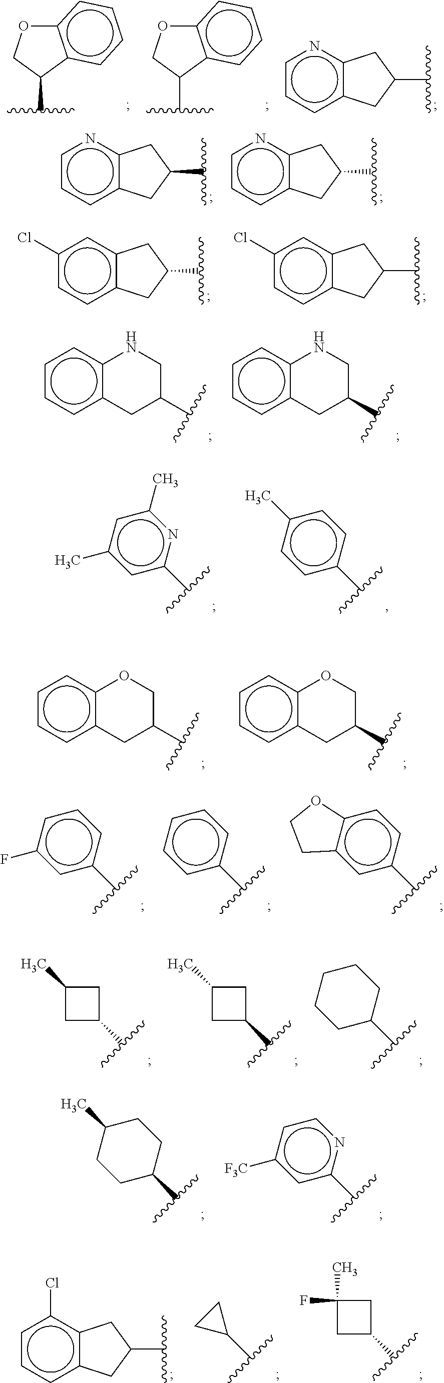

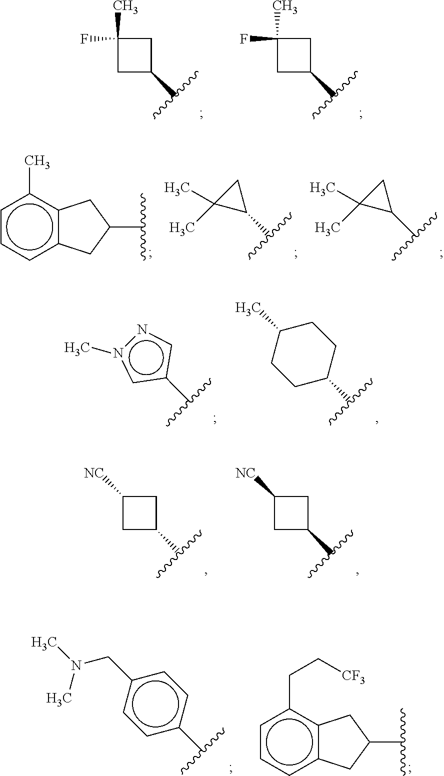

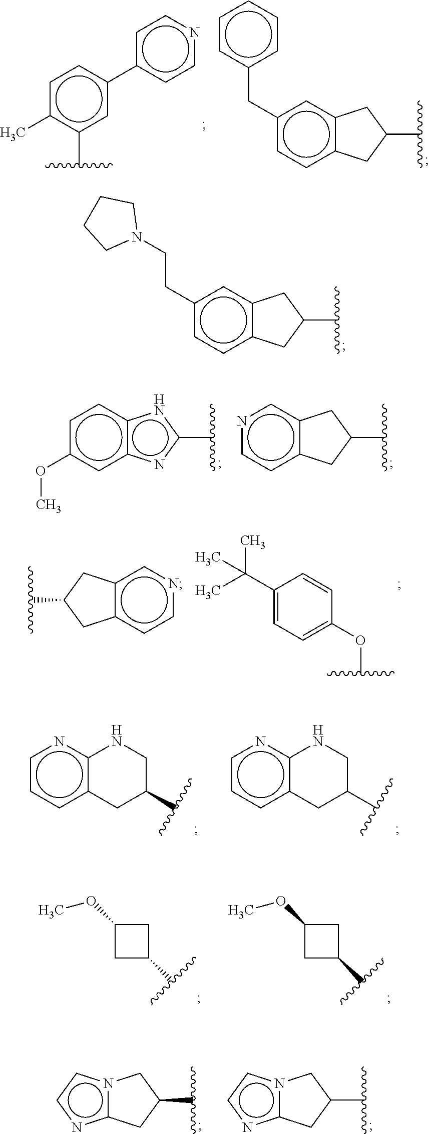

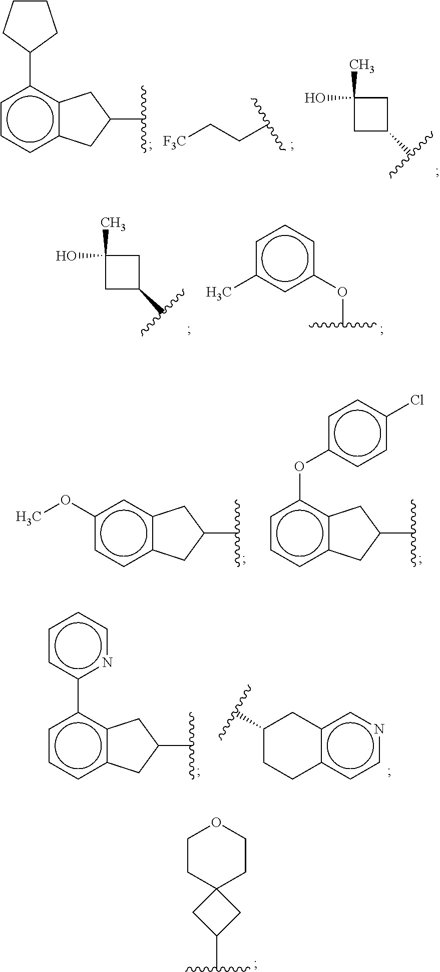

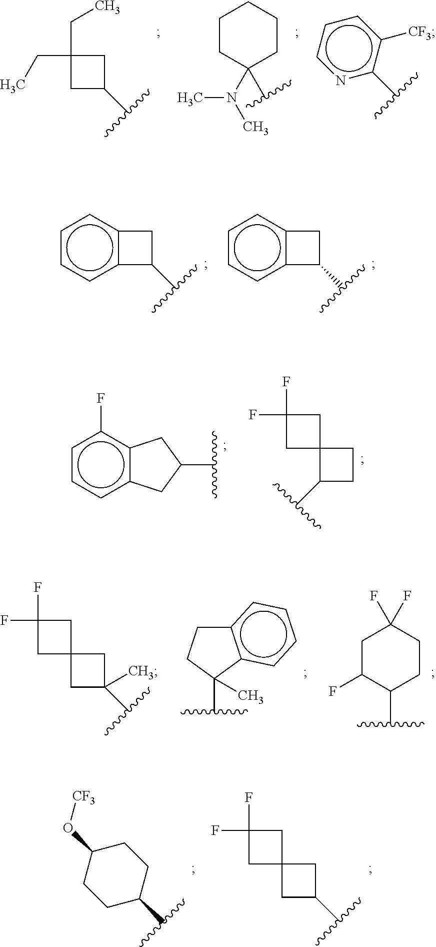

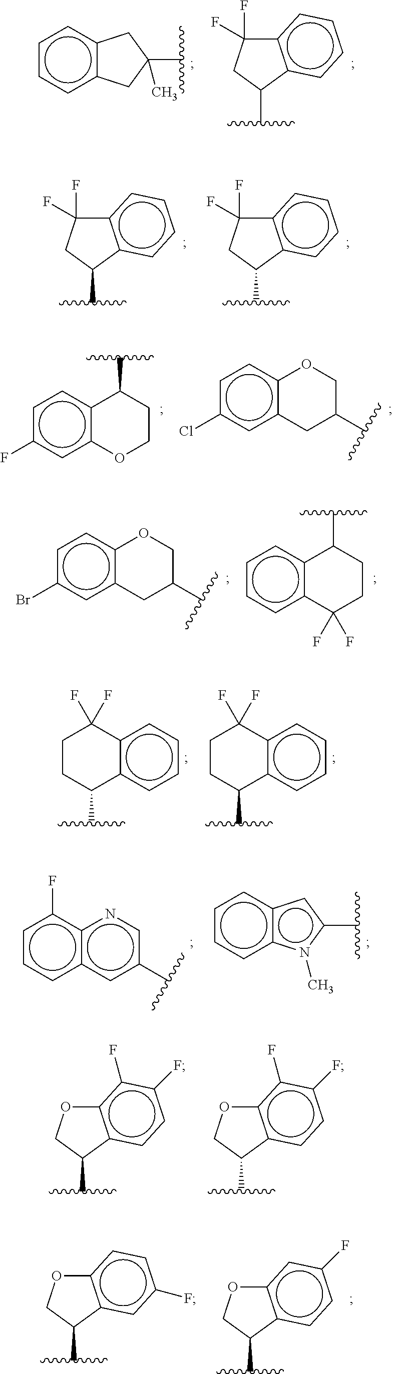

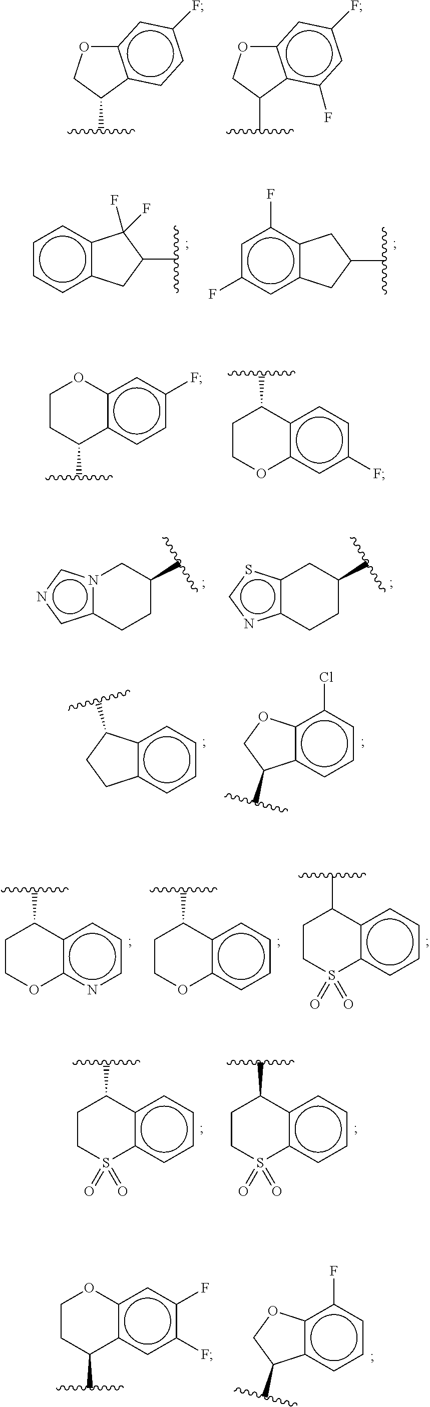

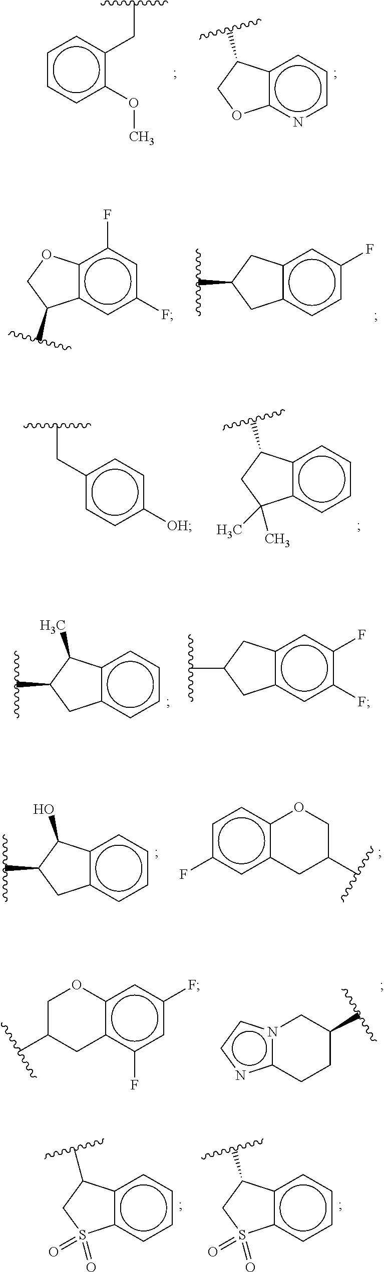

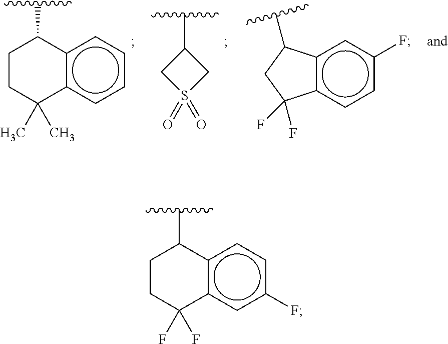

wherein [symbol] indicates the point of attachment to the rest of the compound.

Clause 58. The compound of any one of clauses 1 to 10, 20 to 33, 36, 39, 46 to 49 or 56, wherein
R¹ is selected from the following groups:

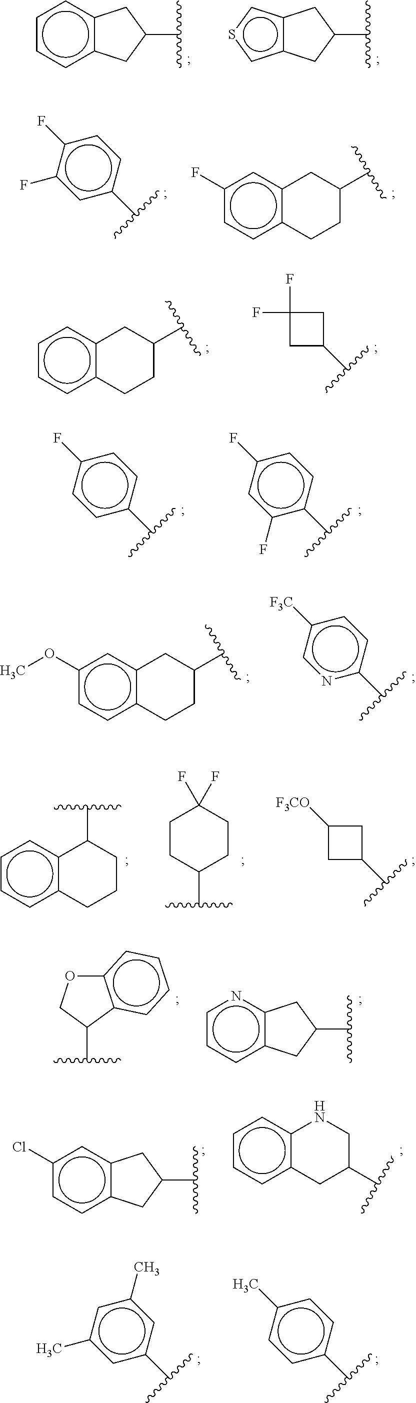

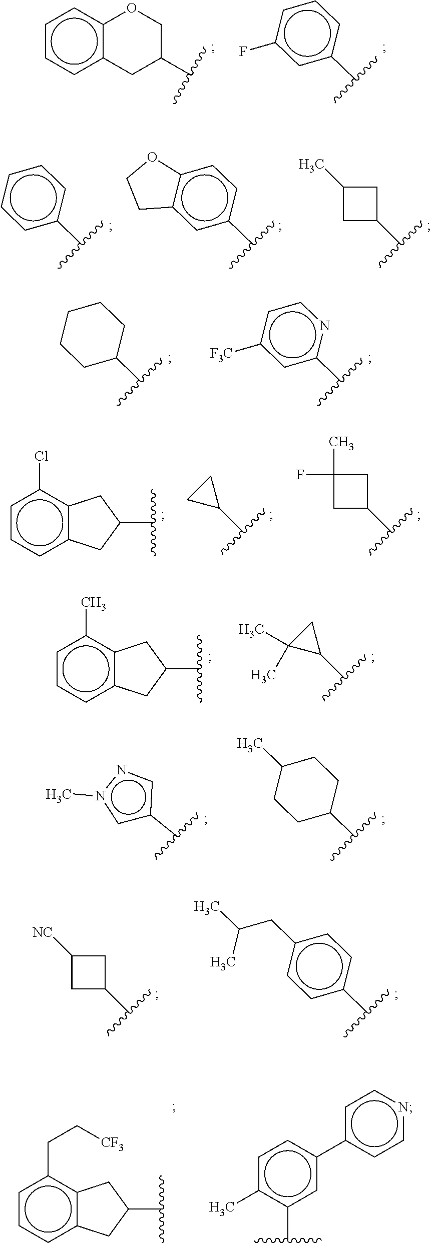

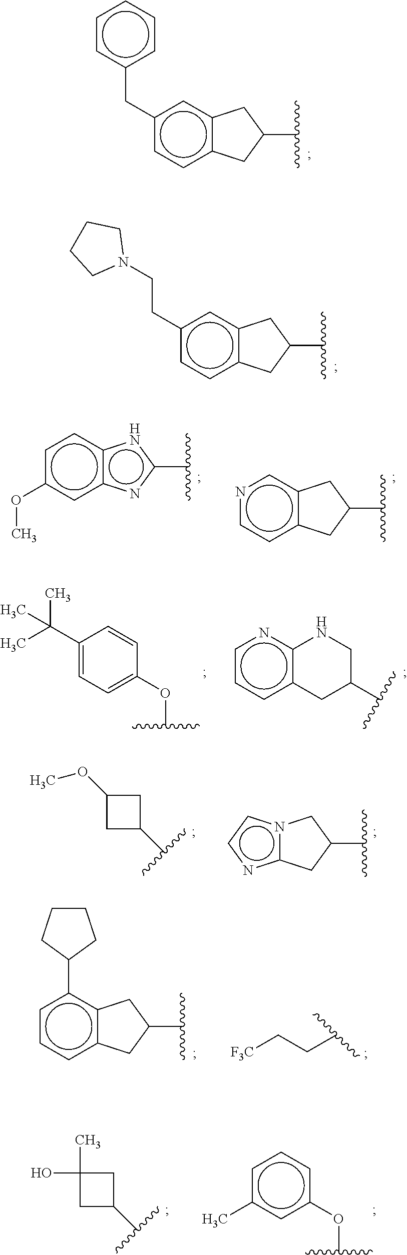

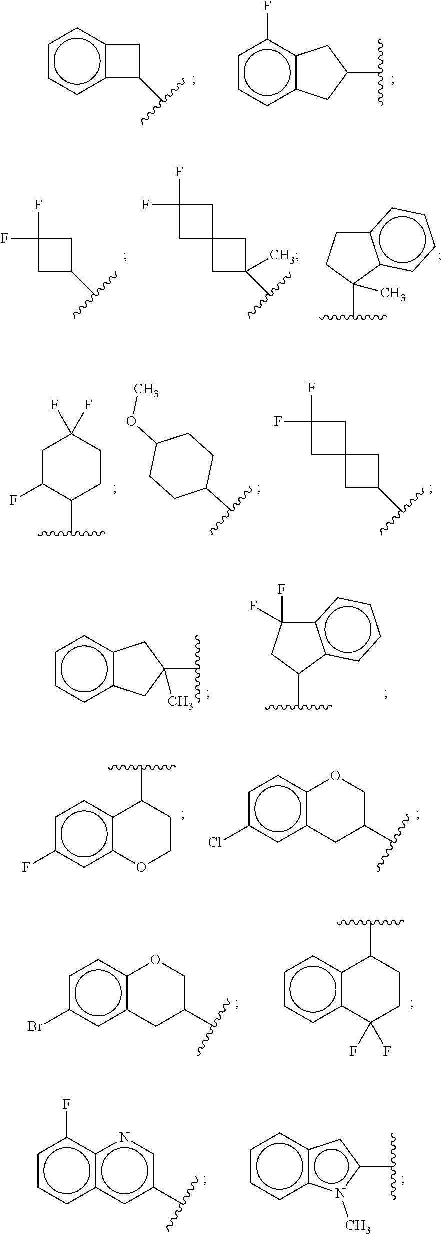

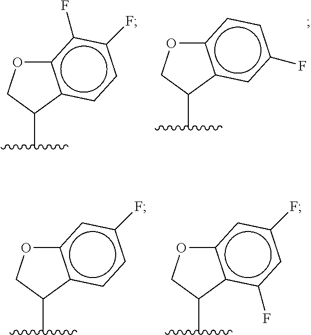

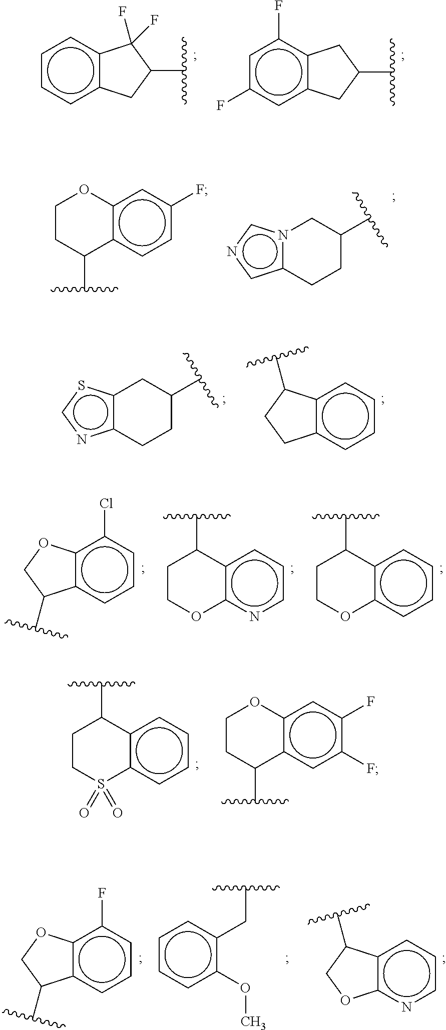

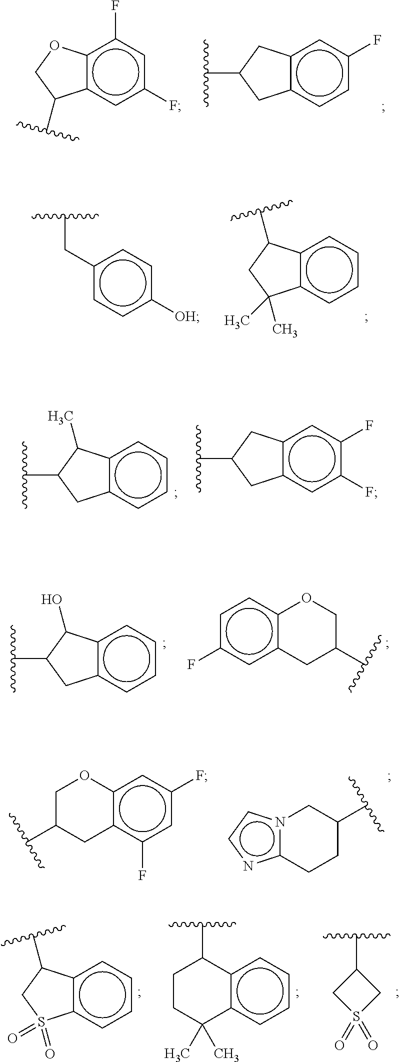

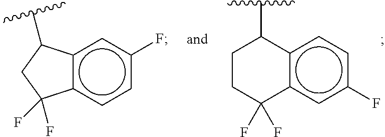

wherein [symbol] indicates the point of attachment to the rest of the compound.

Clause 59. The compound of any one of clauses 1 to 10, 20 to 33, 36, 39, 46 to 49 or 56 to 58, wherein
R¹ is selected from the following groups:

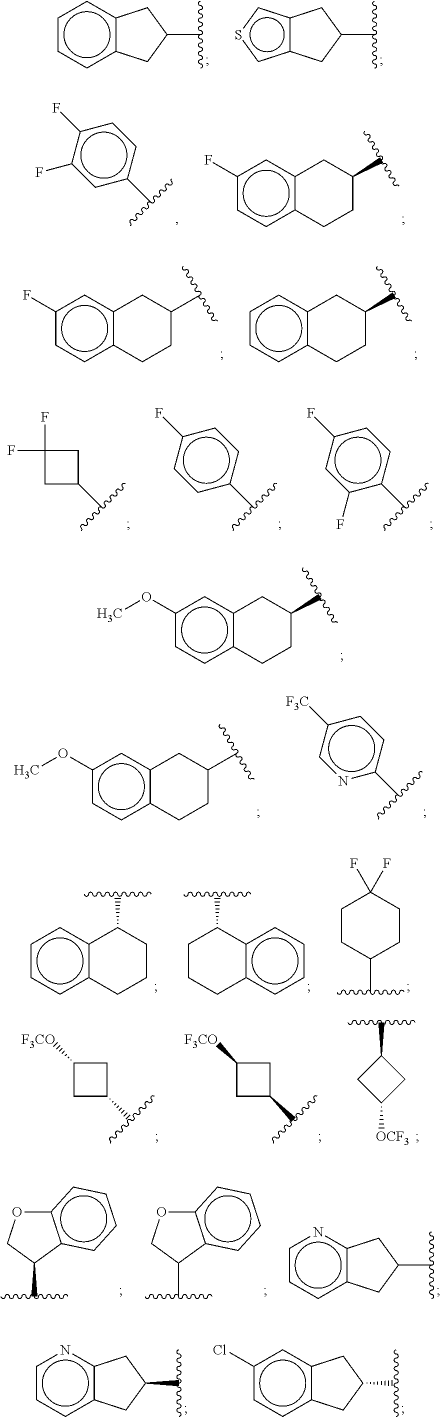

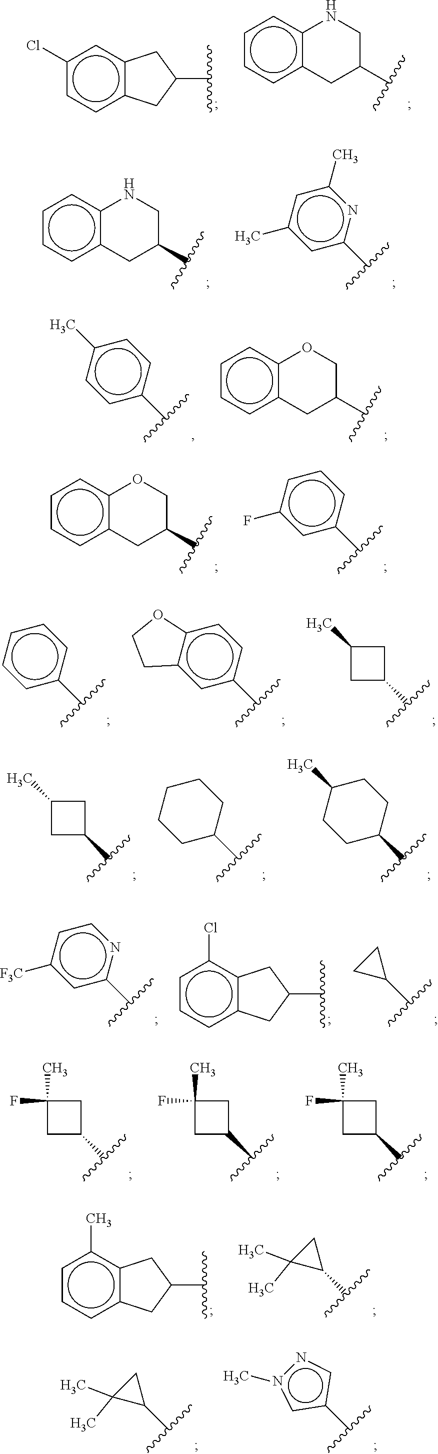

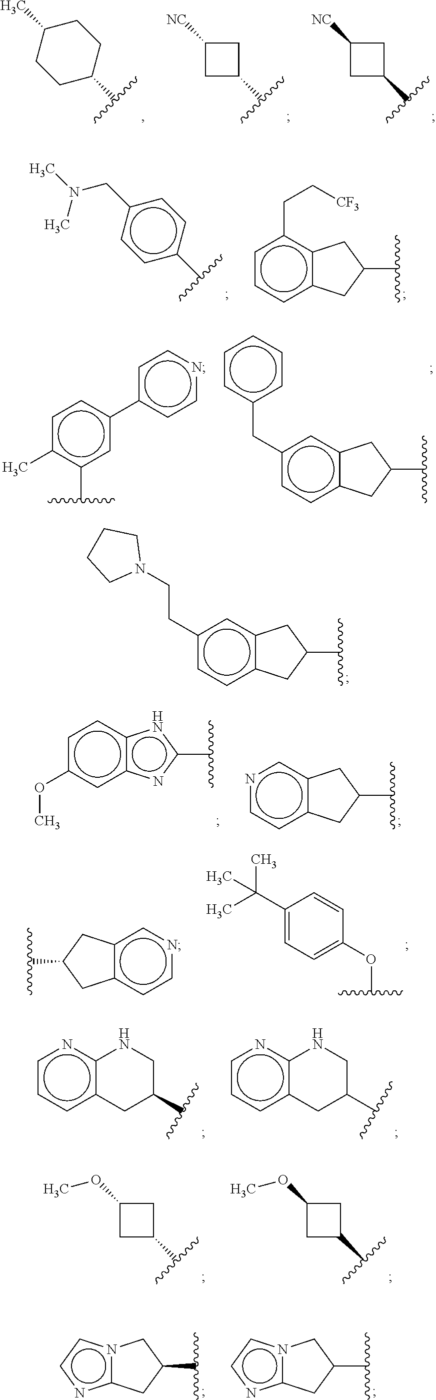

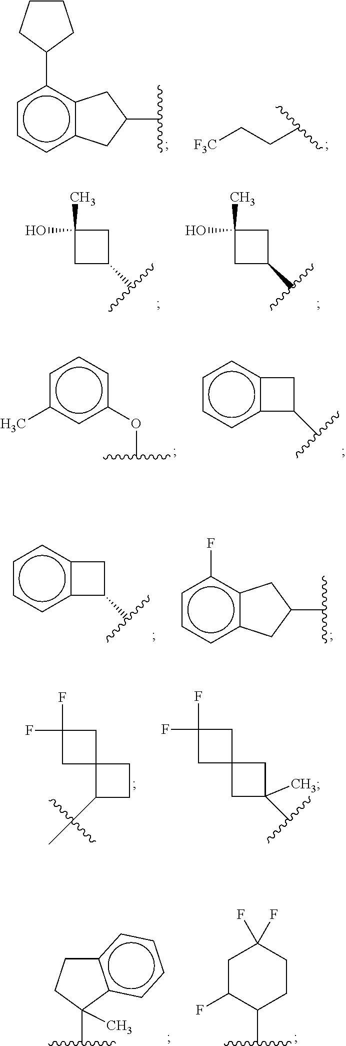

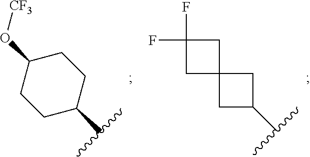 [SMILES: *[C@H]1CC[C@@H](OC(F)(F)F)CC1 ; *C1CC2(C1)CC(F)(F)C2]

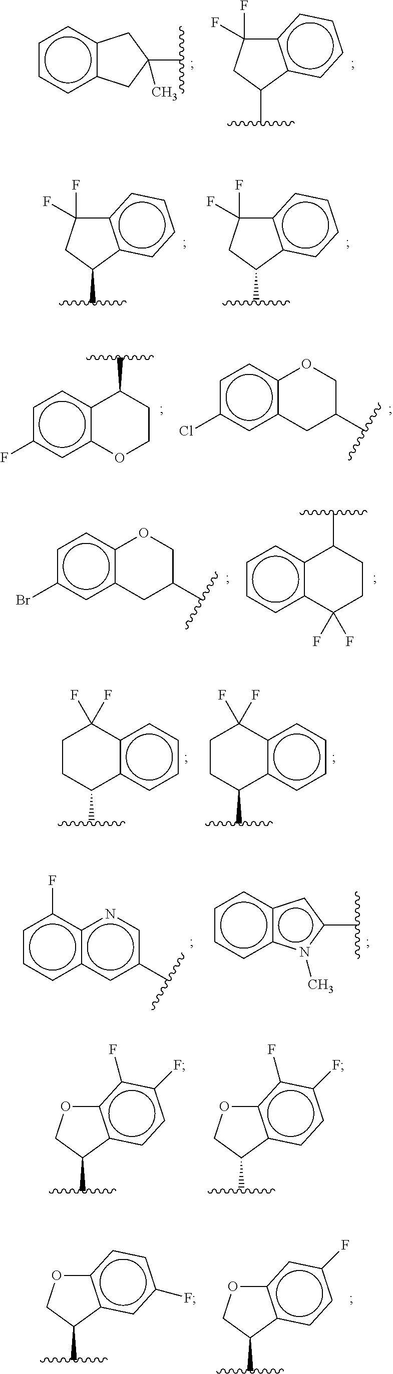

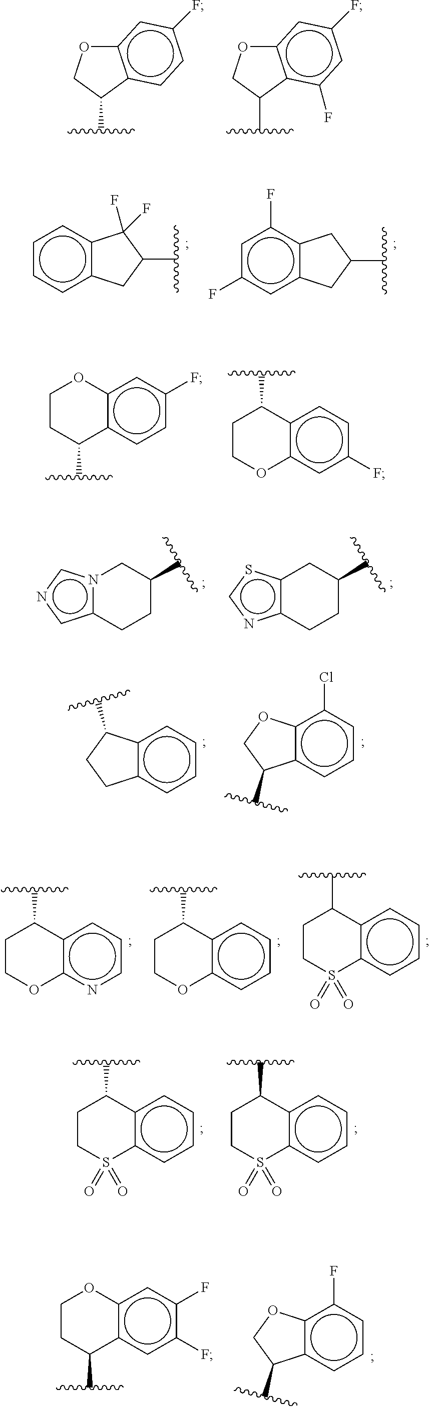

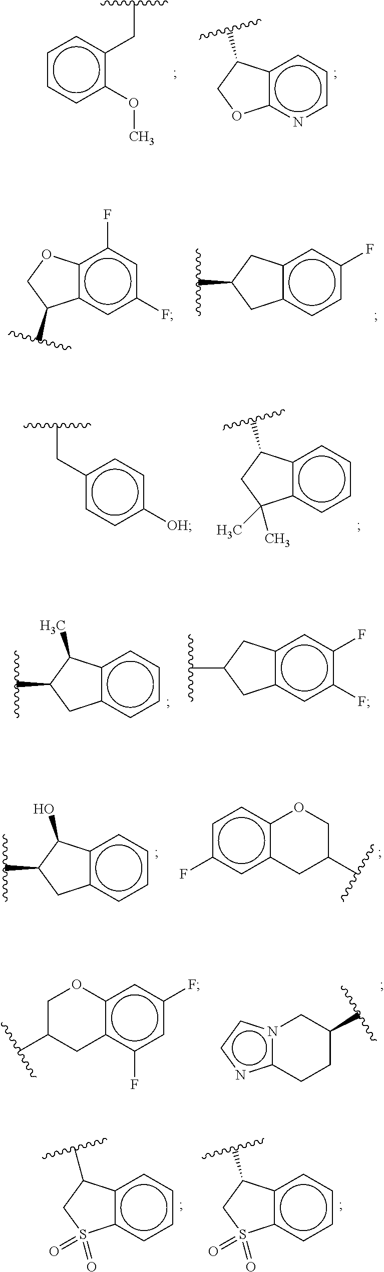

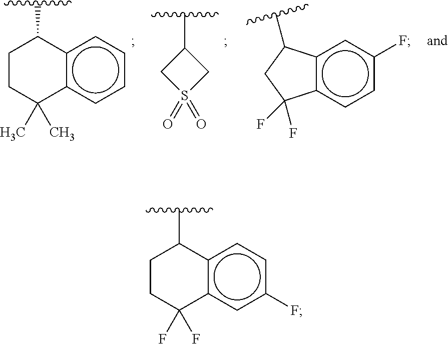

wherein [symbol] indicates the point of attachment to the rest of the compound.

Clause 60. The compound of any one of clauses 1 to 13, 20 to 34, 36, 37, 39, 40, 42, 46 to 51, 56 or 58, wherein
R¹ is selected from the following groups:

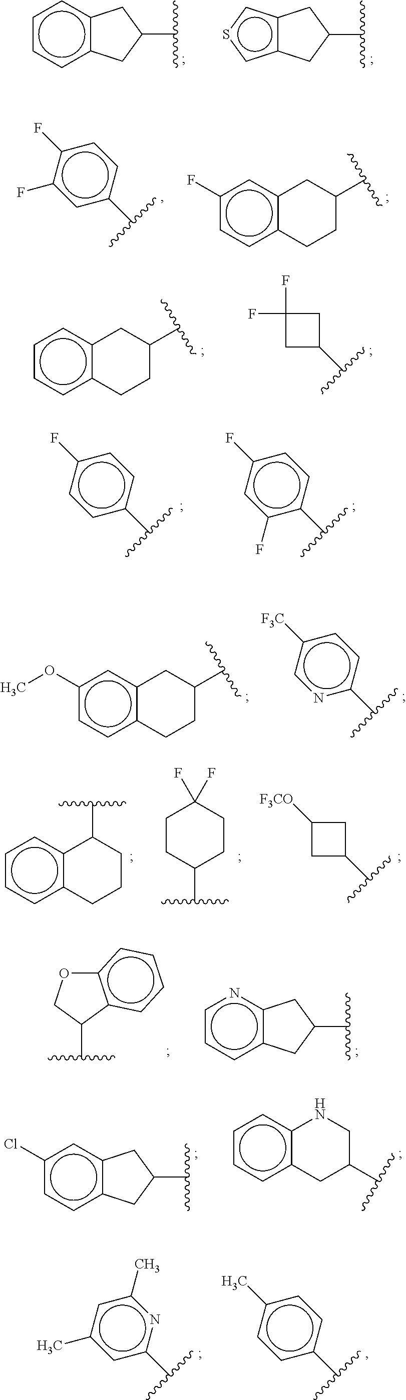

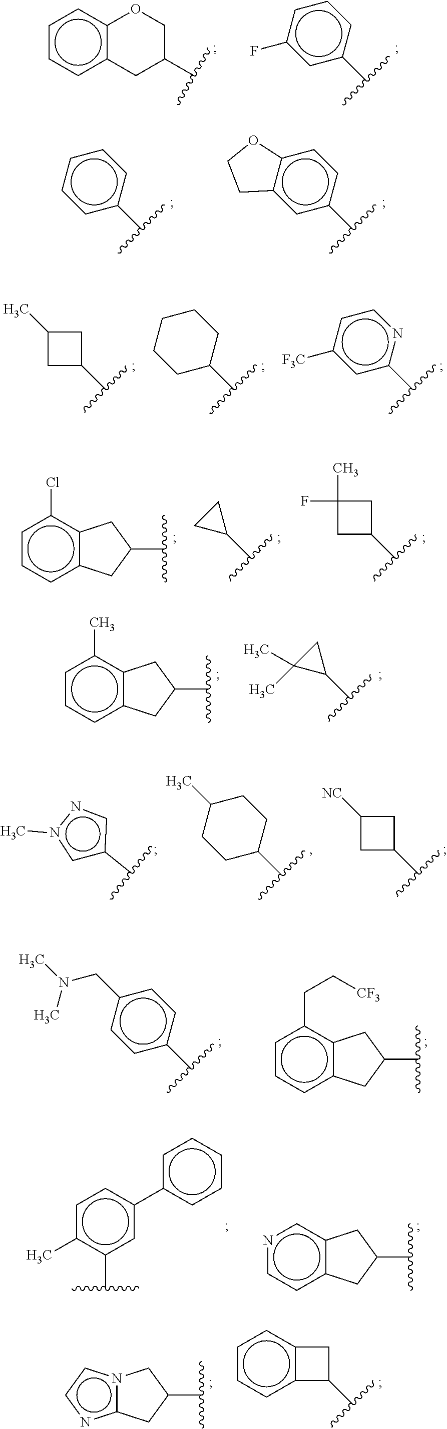

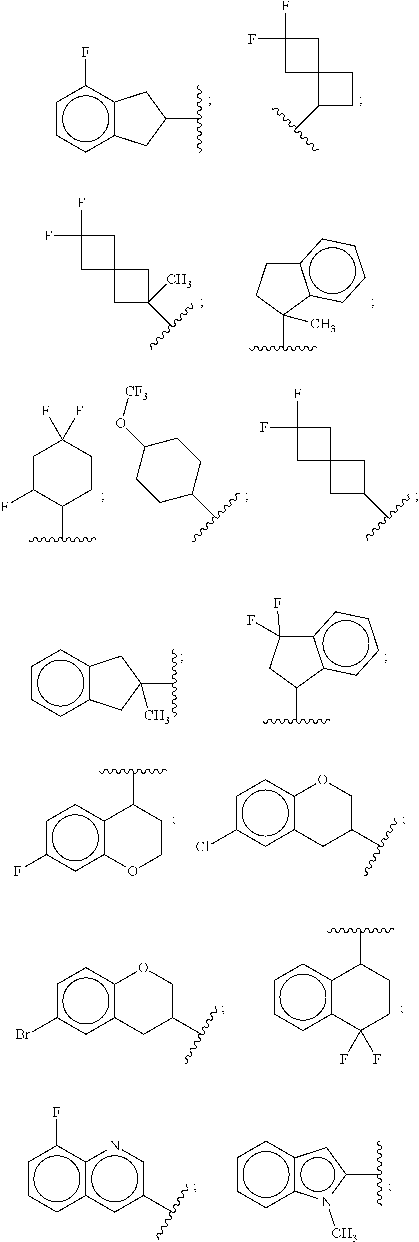

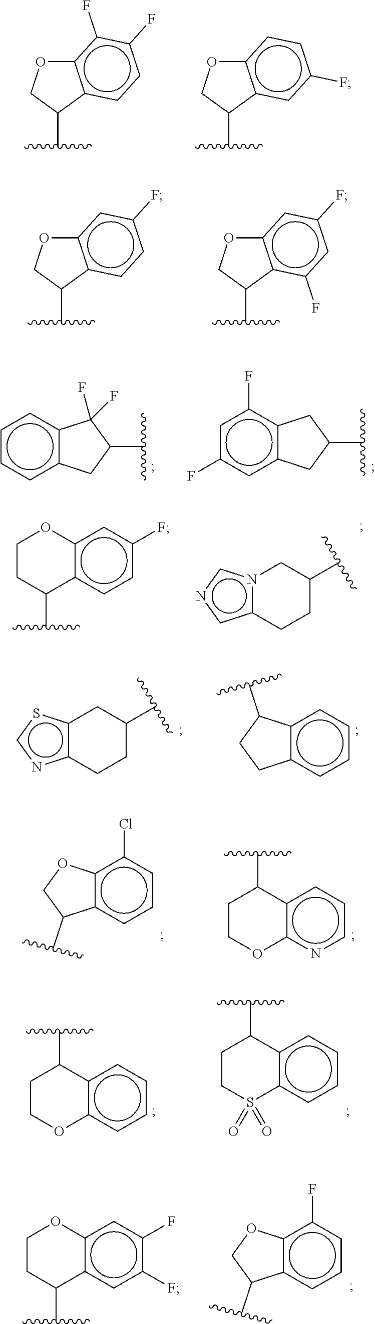

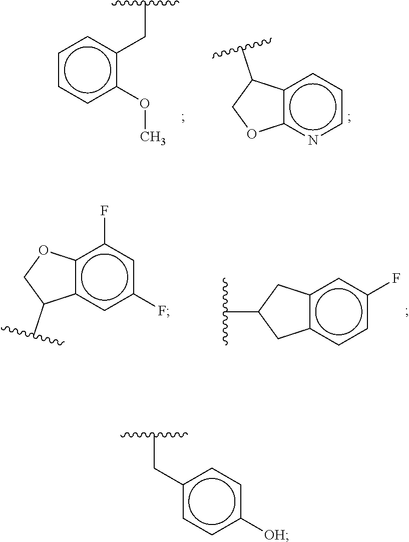

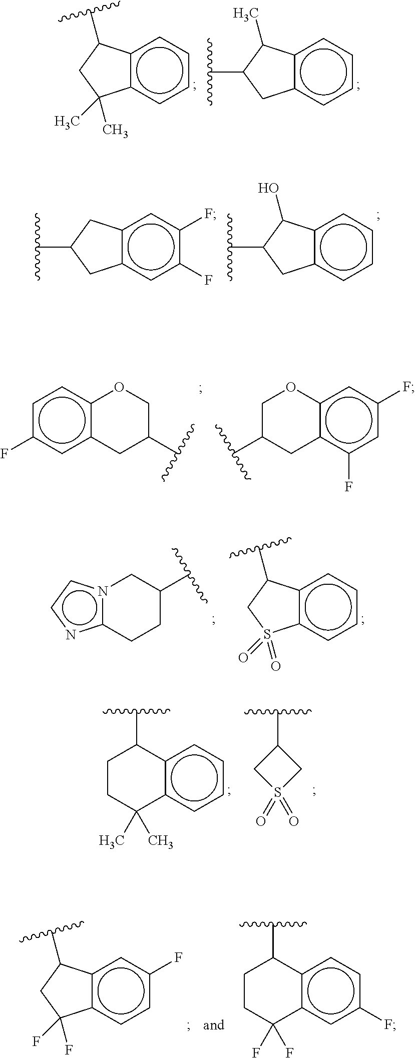

wherein [symbol] indicates the point of attachment to the rest of the compound.

Clause 61. The compound of any one of clauses 1 to 13, 20 to 24, 36, 37, 39, 40, 42, 46 to 51 or 56 to 60, wherein
R¹ is selected from the following groups:

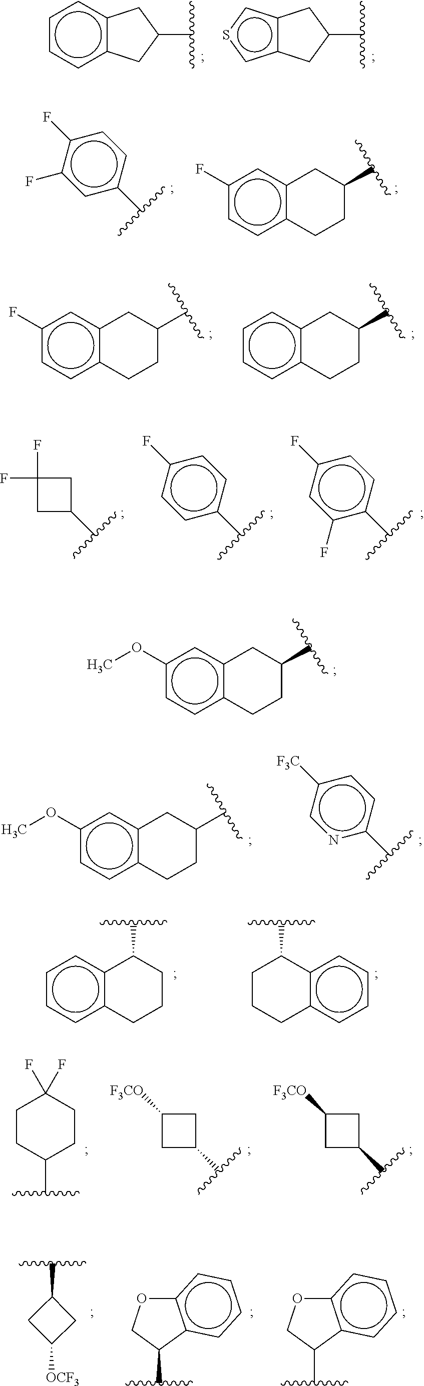

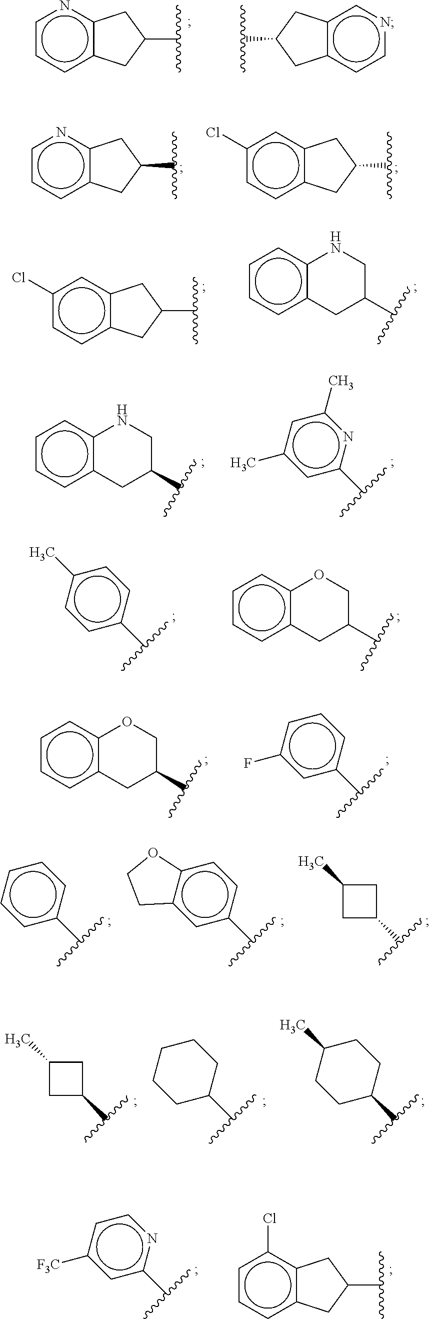

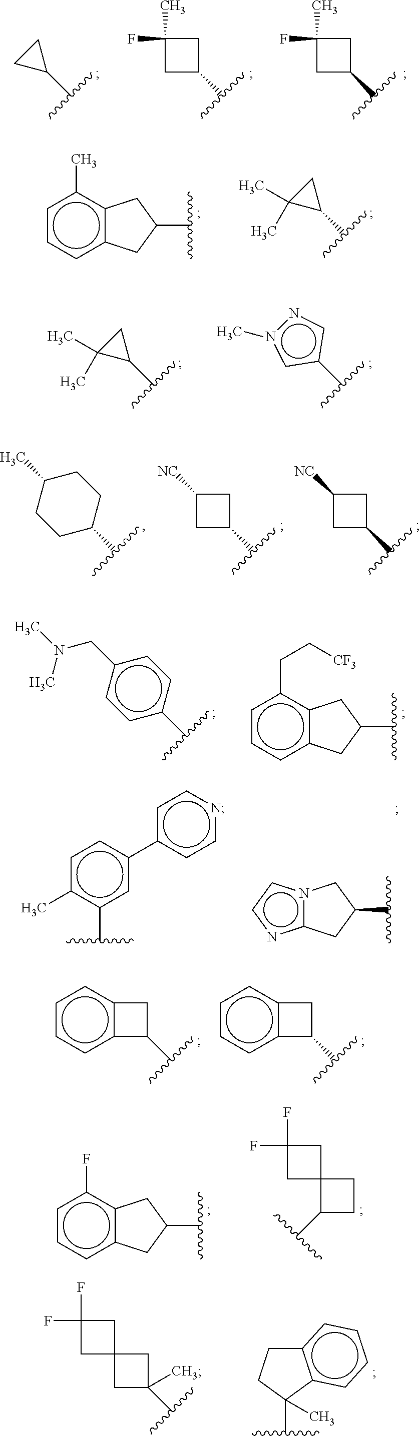

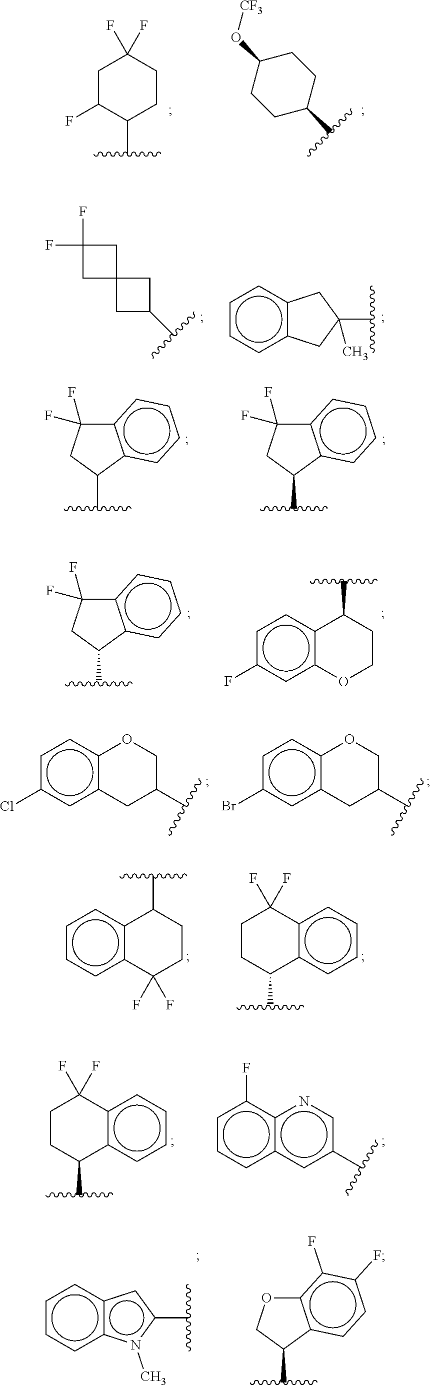

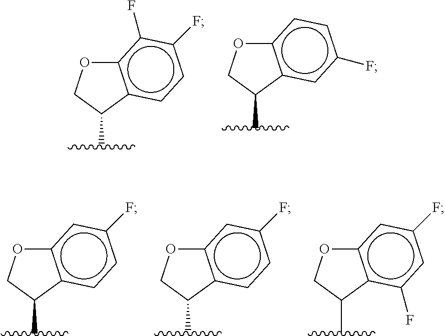

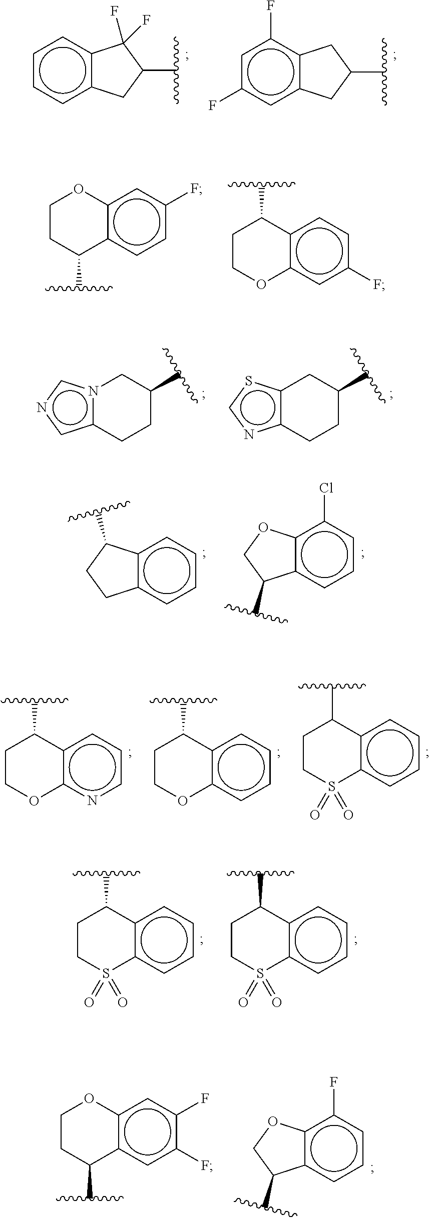

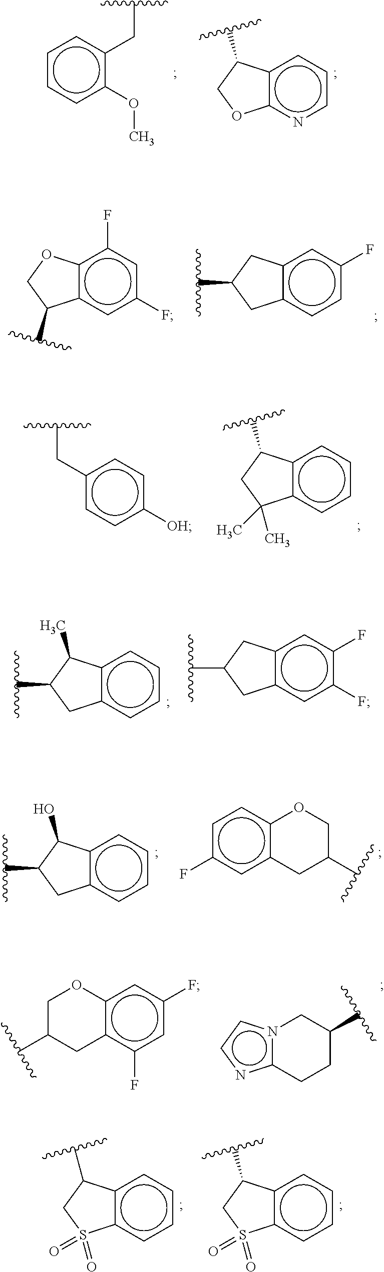

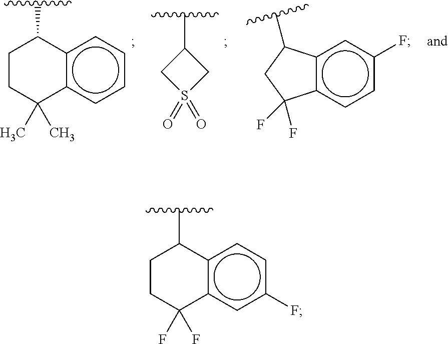

wherein [symbol] indicates the point of attachment to the rest of the compound.

Clause 62. The compound of any one of clauses 1 to 16, 20 to 34, 36, 37, 39, 40, 42, 44, 46 to 53, 56, 58 or 60, wherein
R¹ is selected from the following groups:

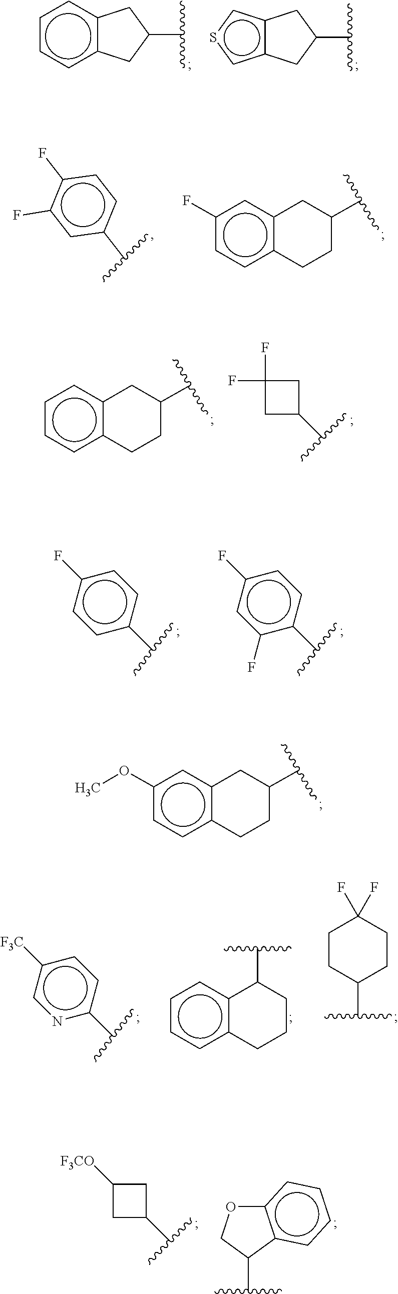

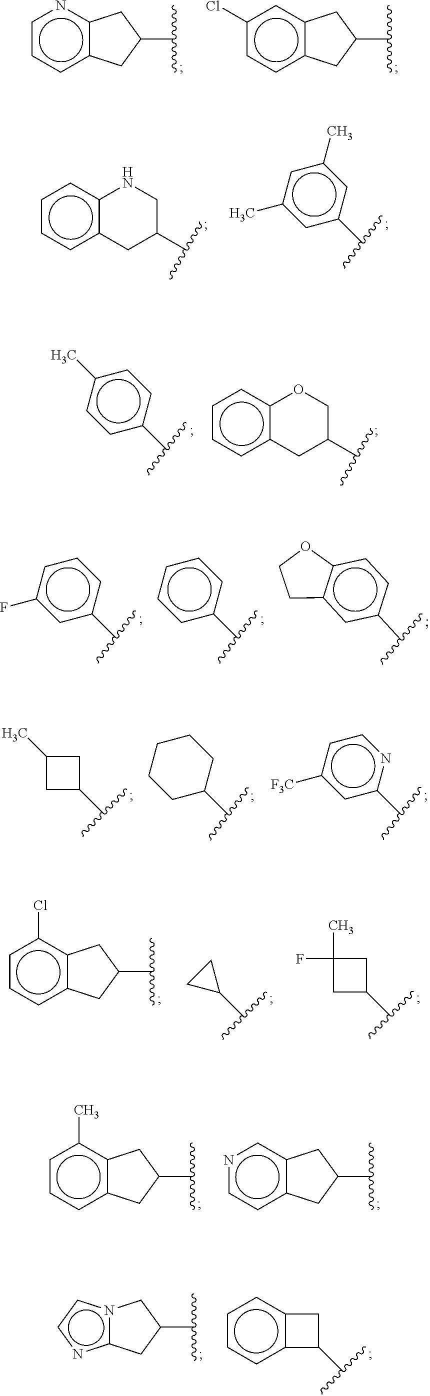

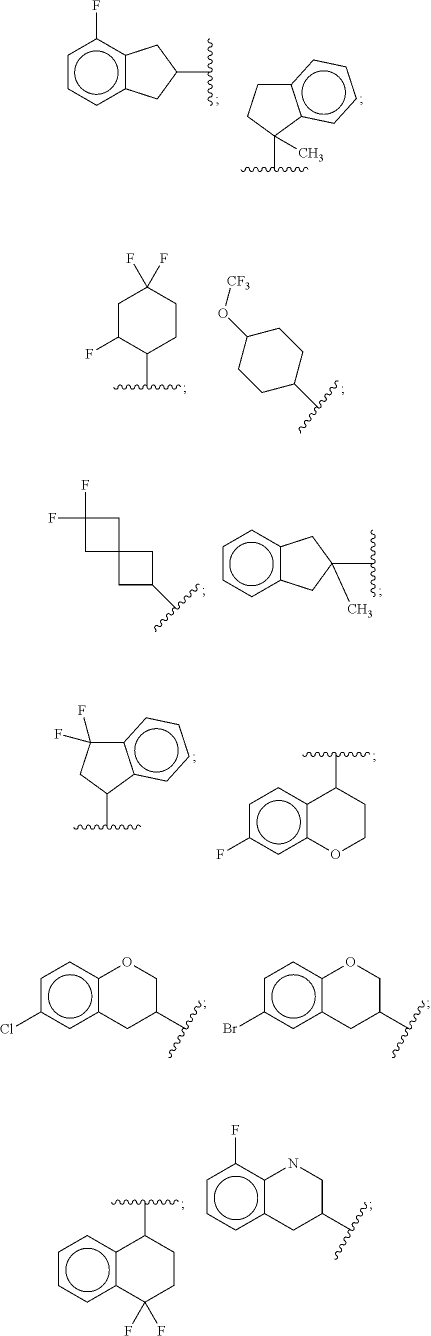

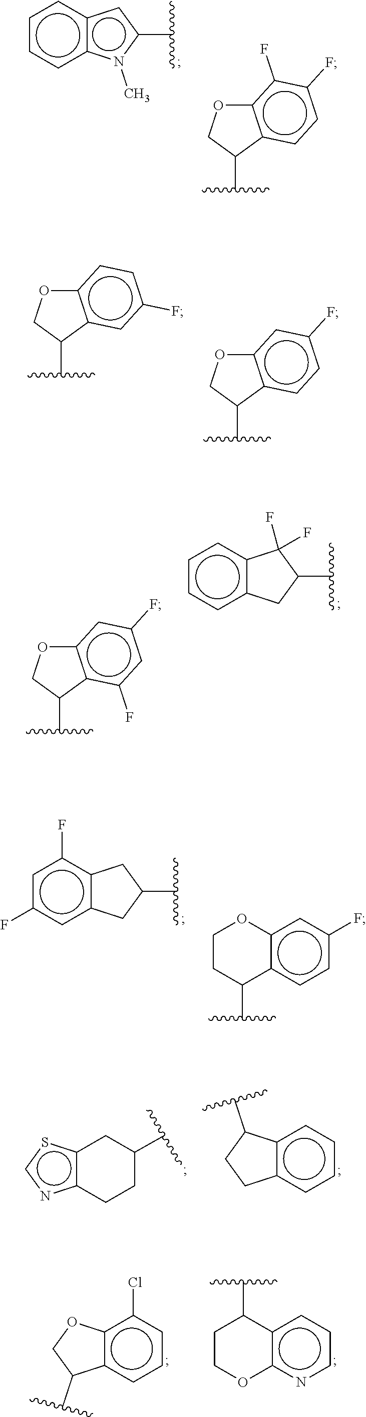

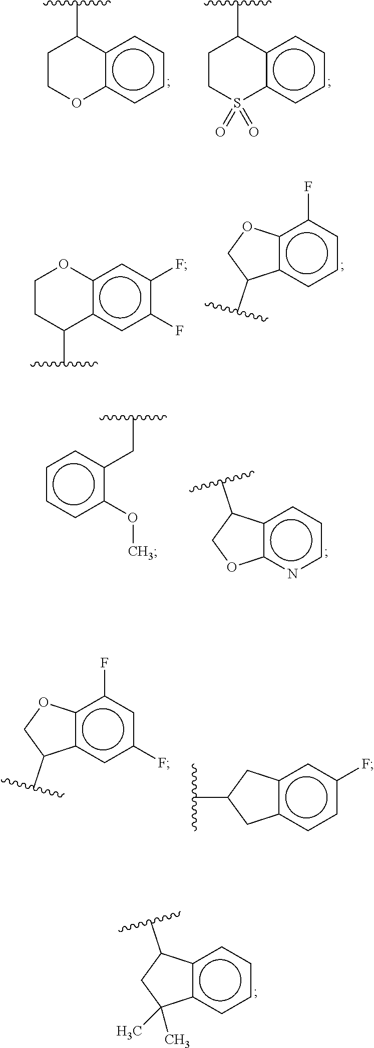

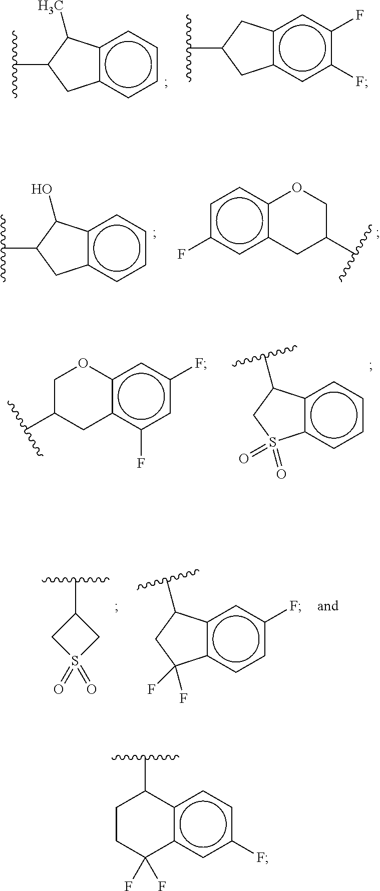

wherein [symbol] indicates the point of attachment to the rest of the compound.

Clause 63. The compound of any one of clauses 1 to 16, 20 to 34, 36, 37, 39, 40, 42, 44, 46 to 53 or 56 to 62, wherein
R¹ is selected from the following groups:

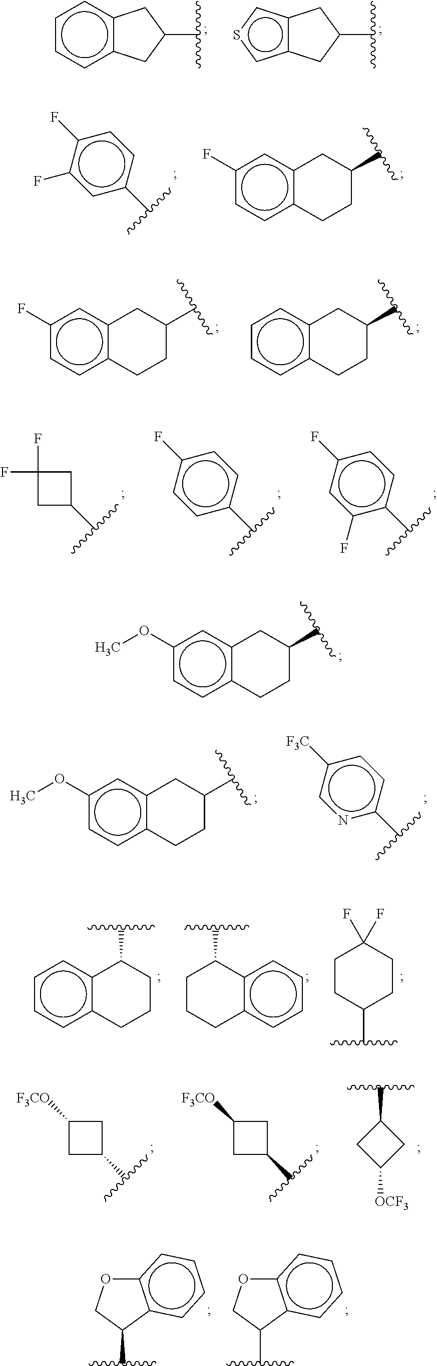

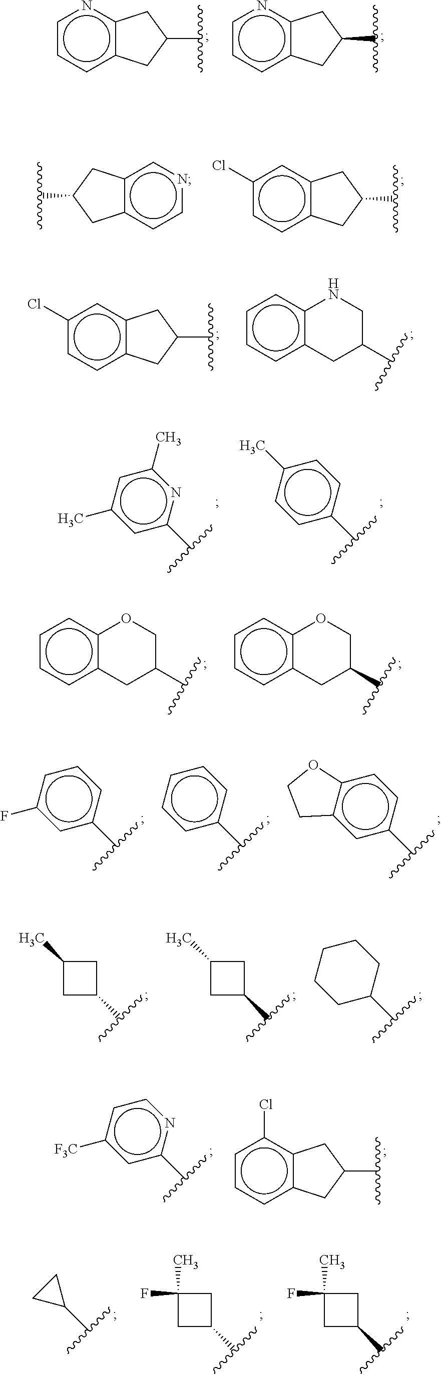

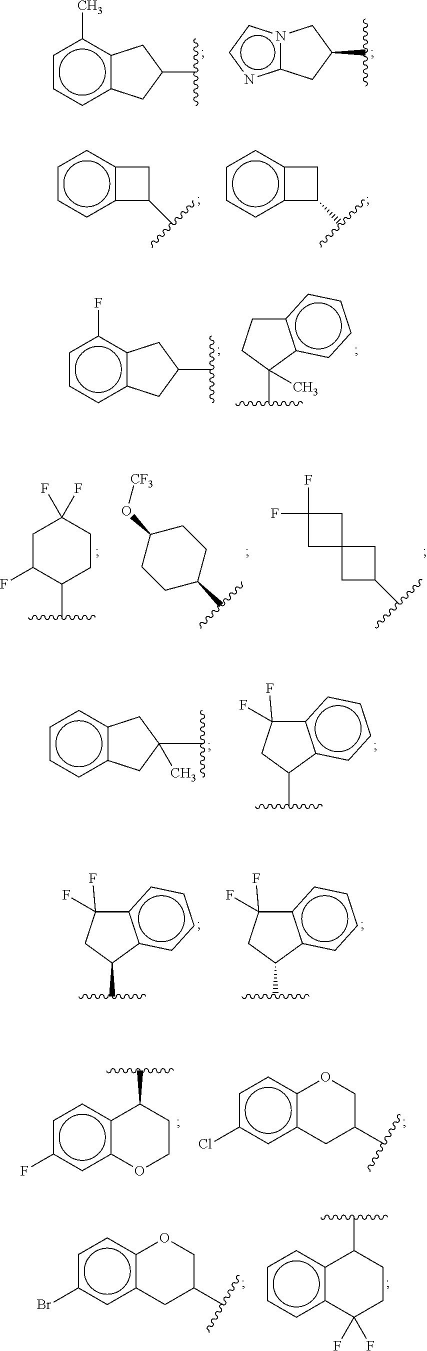

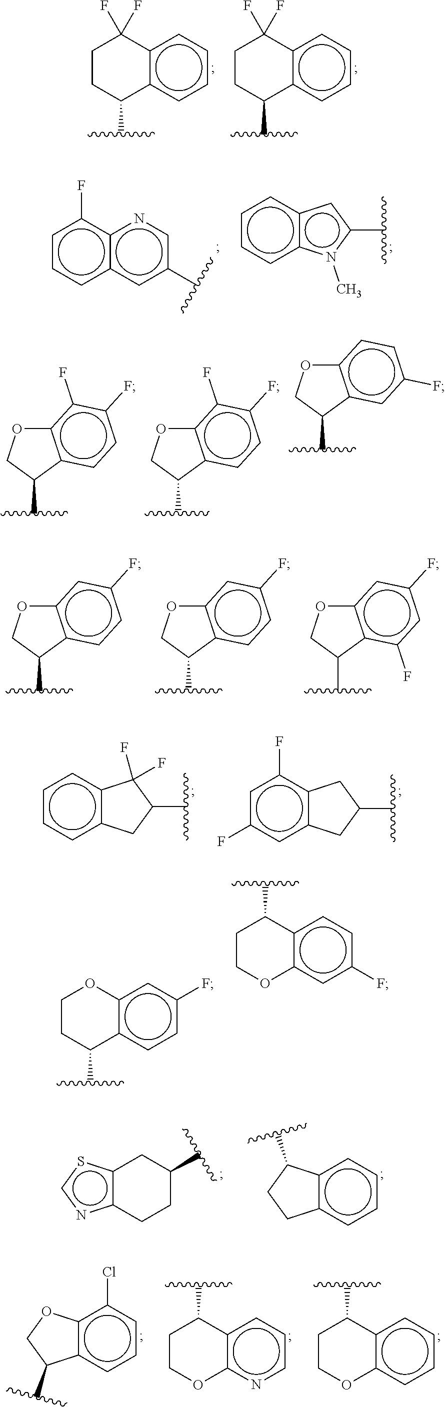

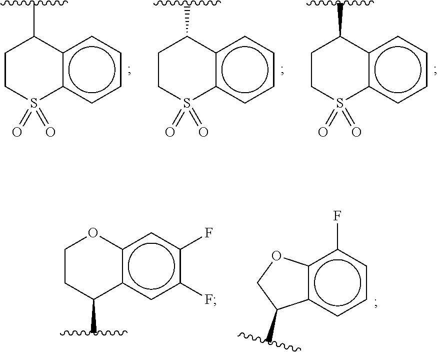

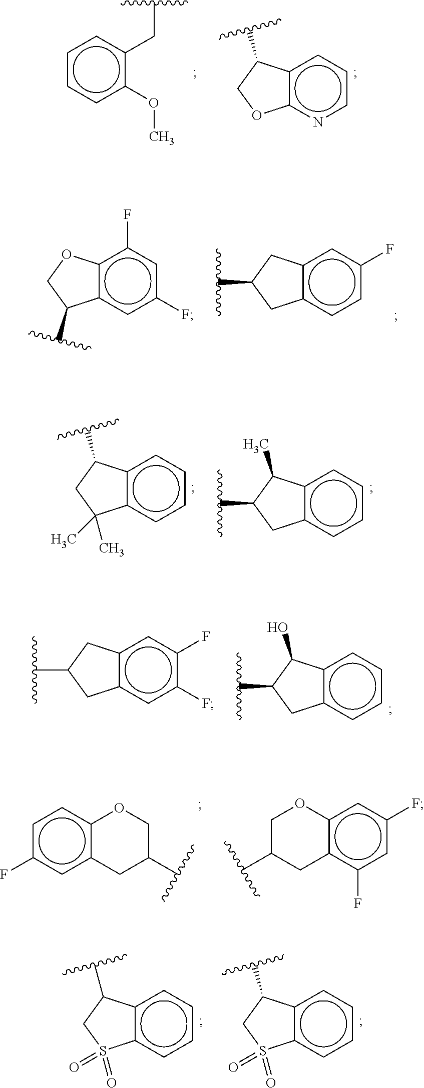

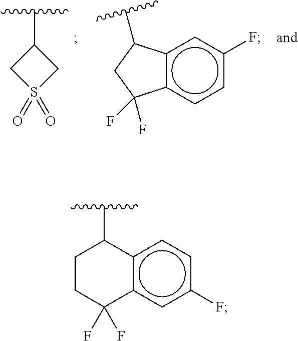

wherein [symbol] indicates the point of attachment to the rest of the compound.

Clause 64. The compound of any one of clauses 1 to 56, 58, 60, 62, wherein
R¹ is selected from the following groups:

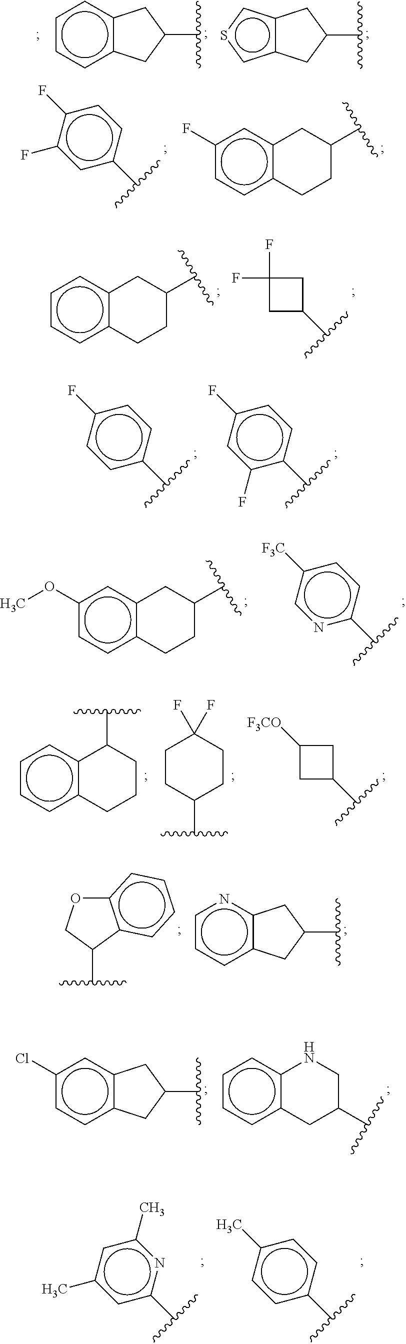

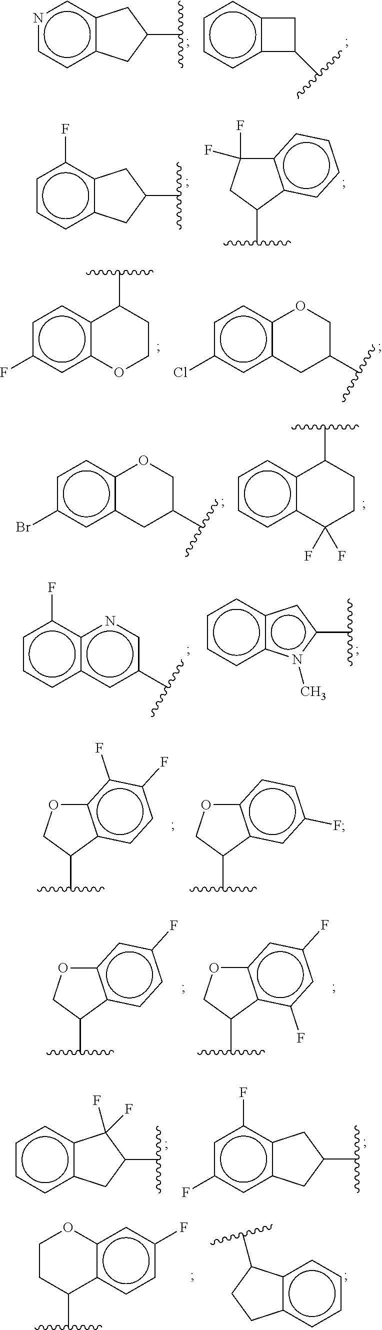

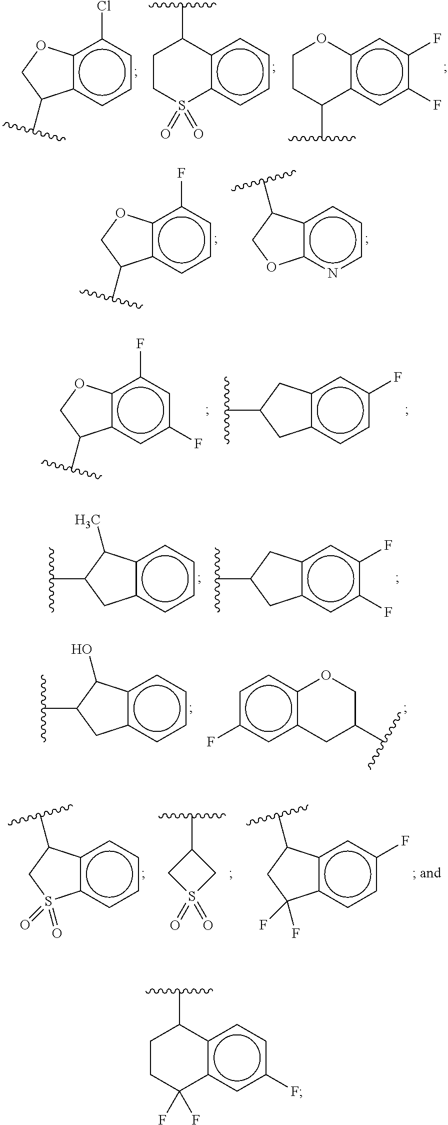

wherein [symbol] indicates the point of attachment to the rest of the compound.

Clause 65. The compound of any preceding clause, wherein
R¹ is selected from the following groups:

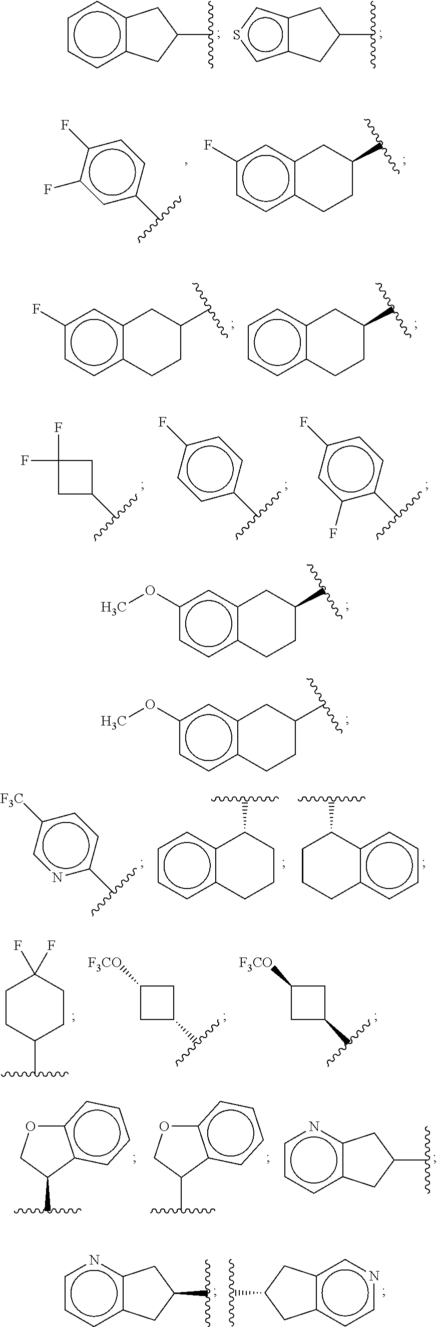

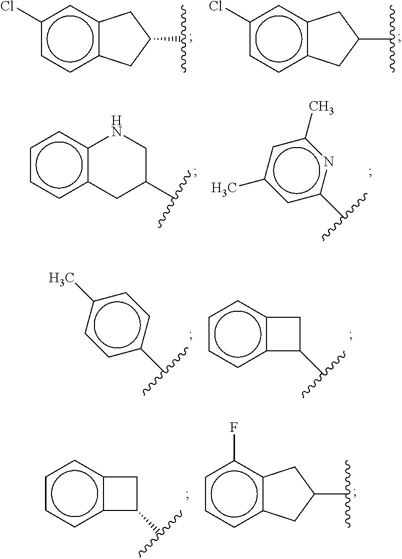

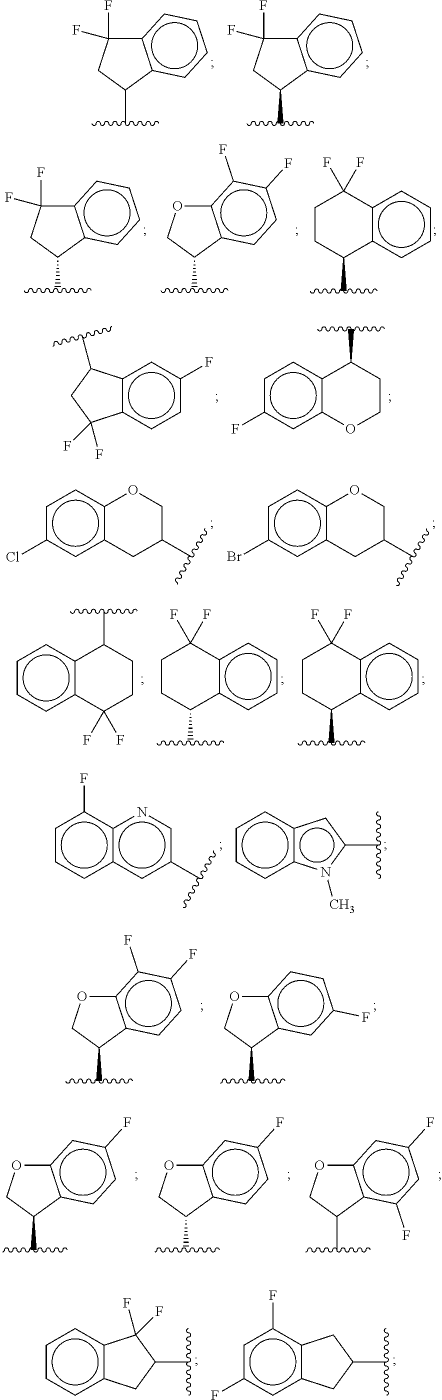

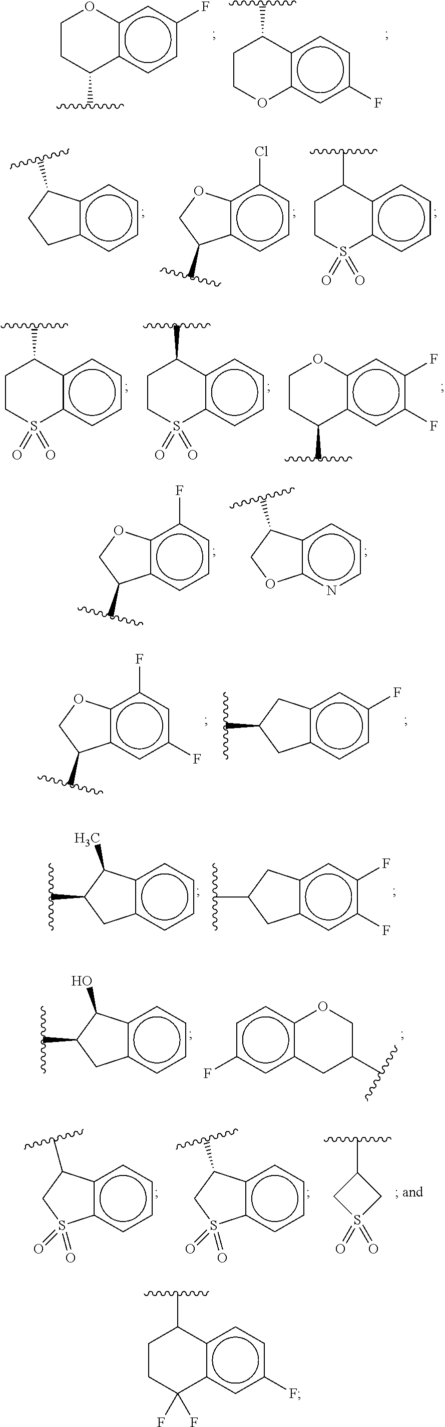

wherein [symbol] indicates the point of attachment to the rest of the compound.

Clause 66. The compound of clause 1 or clause 2, wherein
R¹, R² and L¹ and the nitrogen atom to which R² and L¹ are attached form a heterocycloalkyl, bicyclic heterocycloalkyl, heteroaryl or bicyclic heteroaryl, wherein said heterocycloalkyl, bicyclic heterocycloalkyl, heteroaryl and bicyclic heteroaryl are each optionally substituted by one or more substituents independently selected from halogen, OH, CN, NR^{1a}R^{1b}, alkyl, haloalkyl, alkoxy, haloalkoxy and alkyl-NR^{1a}R^{1b}; and
R^{1a} and R^{1b} are each independently selected from H and alkyl.

Clause 67. The compound of any one of clauses 1, 2 or 66, wherein
R¹, R² and L¹ and the nitrogen atom to which R² and L¹ are attached form a 3- to 8-membered heterocycloalkyl having one or more heteroatoms selected from N, O and S, 8- to 11-membered bicyclic heterocycloalkyl having one or more heteroatoms selected from N, O and S, 5- or 6-membered heteroaryl having one or more heteroatoms selected from N, O and S, or 8- to 10-membered bicyclic heteroaryl having one or more heteroatoms selected from N, O and S, wherein said heterocycloalkyl, bicyclic heterocycloalkyl, heteroaryl and bicyclic heteroaryl are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy and C₁-C₄ alkyl-NR^{1a}R^{1b}; and
R^{1a} and R^{1b} are each independently selected from H and C₁-C₄ alkyl.

Clause 68. The compound of any one of clauses 1, 2, 66 or 67, wherein
R¹, R² and L¹ and the nitrogen atom to which R² and L¹ are attached form a 3- to 8-membered heterocycloalkyl having one or more heteroatoms selected from N, O and S, 8- to 11-membered bicyclic heterocycloalkyl having one or more heteroatoms selected from N, O and S, 5- or 6-membered heteroaryl having one or more heteroatoms selected from N, O and S, or 8- to 10-membered bicyclic heteroaryl having one or more heteroatoms selected from N, O and S.

Clause 69. The compound of any one of clauses 1, 2 or 66 to 68, wherein
R¹, R² and L¹ and the nitrogen atom to which R² and L¹ are attached form a 8- to 11-membered bicyclic heterocycloalkyl having one or more heteroatoms selected from N, O and S.

Clause 70. The compound of any one of clauses 1, 2 or 66 to 69, wherein
R¹, R² and L¹ and the nitrogen atom to which R² and L1 are attached form the following bicyclic heterocycloalkyl group:

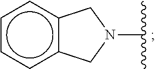

wherein [symbol] indicates the point of attachment to the rest of the compound.

Clause 71. The compound of any one of clauses 1 to 4 or 20 to 65, wherein
R² is selected from H, C₁-C₄ alkyl and C₃-C₈ cycloalkyl.

Clause 72. The compound of any one of clauses 1 to 4 or 20 to 71, wherein
R² is selected from H and C₁-C₄ alkyl.

Clause 73. The compound of any preceding clause, wherein
R² is H.

Clause 74. The compound of any one of clauses 1 to 3 or 20 to 73, wherein
R³ is selected from H, OH, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkoxy-C₁-C₄ alkyl, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, CN, C₁-C₄ alkyl, C₁-C₄ alkoxy, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl.

Clause 75. The compound of any one of clauses 1 to 3 or 20 to 74, wherein
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, CN, C₁-C₄ alkyl, C₁-C₄ alkoxy, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl.

Clause 76. The compound of any one of clauses 1 to 3 or 20 to 75, wherein
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, and heteroaryl are each optionally substituted by one or more substituents independently selected from halogen, OH, CN, C₁-C₄ alkyl, C₁-C₄ alkoxy, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl.

Clause 77. The compound of any one of clauses 1 to 3 or 20 to 76, wherein
R³ is selected from NR^{3a}R^{3b}, C₁-C₄ alkyl, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, and C₁-C₄ alkyl-S—C₁-C₄ alkyl, wherein said alkyl, cycloalkyl, heterocycloalkyl, and heteroaryl are each optionally substituted by one or more substituents independently selected from halogen, OH, CN, C₁-C₄ alkyl, C₁-C₄ alkoxy, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl.

Clause 78. The compound of any one of clauses 1 to 5 or 20 to 75, wherein
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, C₁-C₄ alkyl, and NR^{3c}R^{3d};
R^{3a} is selected from H, C₁-C₄ alkyl, and C₁-C₄ alkyl-NR^{3e}R^{3f};
R^{3b} is selected from H and C₁-C₄ alkyl;
R^{3c} is selected from H and C₁-C₄ alkyl;
R^{3d} is selected from C₁-C₄ alkyl; and
R^{3e} and R^{3f} are each independently selected from C₁-C₄ alkyl.

Clause 79. The compound of any one of clauses 1 to 5 or 20 to 76, wherein
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, and heteroaryl are each optionally substituted by one or more substituents independently selected from halogen, OH, C₁-C₄ alkyl, and NR^{3c}R^{3d};
R^{3a} is selected from H, C₁-C₄ alkyl, and C₁-C₄ alkyl-NR^{3e}R^{3f};
R^{3b} is selected from H and C₁-C₄ alkyl;
R^{3c} is selected from H and C₁-C₄ alkyl;
R^{3d} is selected from C₁-C₄ alkyl; and
R^{3e} and R^{3f} are each independently selected from C₁-C₄ alkyl.

Clause 80. The compound of any one of clauses 1 to 5 or 20 to 79, wherein
R³ is selected from NR^{3a}R^{3b}, C₁-C₄ alkyl, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, and C₁-C₄ alkyl-S—C₁-C₄ alkyl, wherein said alkyl, cycloalkyl, heterocycloalkyl, and heteroaryl are each optionally substituted by one or more substituents independently selected from halogen, OH, C₁-C₄ alkyl, and NR^{3c}R^{3d};
R^{3a} is selected from H, C₁-C₄ alkyl, and C₁-C₄ alkyl-NR^{3e}R^{3f};
R^{3b} is selected from H and C₁-C₄ alkyl;
R^{3c} is selected from H and C₁-C₄ alkyl;
R^{3d} is selected from C₁-C₄ alkyl; and
R^{3e} and R^{3f} are each independently selected from C₁-C₄ alkyl.

Clause 81. The compound of any one of clauses 1 to 5, 8, 20 to 75, or 78 wherein
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH and NR^{3c}R^{3d};
R^{3a} is selected from H and C₁-C₄ alkyl;
R^{3b} is selected from C₁-C₄ alkyl;
R^{3c} is selected from H and C₁-C₄ alkyl; and
R^{3d} is selected from C₁-C₄ alkyl.

Clause 82. The compound of any one of clauses 1 to 5, 8, 20 to 76, 78, 79, or 81, wherein
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₃-C₈ cycloalkoxy-C₁-C₄ alkyl, C₃-C₈ cycloalkyl-C₁-C₄ alkoxy, C₁-C₄ haloalkoxy-C₁-C₄ alkyl, C₁-C₄ alkyl-S—C₁-C₄ alkyl, and C₁-C₄ alkyl-SO₂-C₁-C₄ alkyl, wherein said alkyl, cycloalkyl, heterocycloalkyl, and heteroaryl are each optionally substituted by one or more substituents independently selected from halogen, OH and NR^{3c}R^{3d};
R^{3a} is selected from H and C₁-C₄ alkyl;
R^{3b} is selected from C₁-C₄ alkyl;
R^{3c} is selected from H and C₁-C₄ alkyl; and
R^{3d} is selected from C₁-C₄ alkyl.

Clause 83. The compound of any one of clauses 1 to 5, 8 or 20 to 82, wherein
R³ is selected from NR^{3a}R^{3b}, C₁-C₄ alkyl, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₃-C₈ cycloalkoxy-C₁-C₄ alkyl, C₃-C₈ cycloalkyl-C₁-C₄ alkoxy, C₁-C₄ haloalkoxy-C₁-C₄ alkyl, and C₁-C₄ alkyl-S—C₁-C₄ alkyl, wherein said alkyl, cycloalkyl, heterocycloalkyl, and heteroaryl are each optionally substituted by one or more substituents independently selected from halogen, OH and NR^{3c}R^{3d};
R^{3a} is selected from H and C₁-C₄ alkyl;
R^{3b} is selected from C₁-C₄ alkyl;
R^{3c} is selected from H and C₁-C₄ alkyl; and
R^{3d} is selected from C₁-C₄ alkyl.

Clause 84. The compound of any one of clauses 1 to 5, 8, 11, 14, 20 to 76, 78, 79, 81, or 82, wherein
R³ is selected from H, CN, NR^{3a}R^{3b}, C₁-C₄ alkyl optionally substituted by OH, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₃-C₈ cycloalkoxy-C₁-C₄ alkyl, C₃-C₈ cycloalkyl-C₁-C₄ alkoxy, C₁-C₄ haloalkoxy-C₁-C₄ alkyl, C₁-C₄ alkyl-S—C₁-C₄ alkyl, and C₁-C₄ alkyl-SO₂-C₁-C₄ alkyl;
R^{3a} is H; and
R^{3b} is selected from C₁-C₄ alkyl.

Clause 85. The compound of any one of clauses 1 to 5, 8, 11, 14, 17, or 20 to 84, wherein
R³ is selected from NR^{3a}R^{3b}, C₁-C₄ alkyl substituted by one or more substituents independently selected from OH and NR^{3c}R^{3d}, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₃-C₈ cycloalkoxy-C₁-C₄ alkyl, C₃-C₈ cycloalkyl-C₁-C₄ alkoxy, C₁-C₄ haloalkoxy-C₁-C₄ alkyl, and C₁-C₄ alkyl-S—C₁-C₄ alkyl;
R^{3a} is H;
R^{3b} is selected from C₁-C₄ alkyl.

Clause 86. The compound of any one of clauses 1 to 7, 20 to 75, or 78, wherein
R³ is selected from:
   H;
   CN;
   NR^{3a}R^{3b};
   C₁-C₄ alkyl optionally substituted by one of more substituents selected from OH and NR^{3c}R^{3d};
   C₃-C₈ cycloalkyl;
   3- to 8-membered heterocycloalkyl having one or more heteroatoms selected from N, O and S, optionally substituted by one or more C₁-C₄ alkyl;
   phenyl optionally substituted by one or more halogen;
   5- or 6-membered heteroaryl having one or more heteroatoms selected from N, O and S;
   C₁-C₄ alkoxy-C₁-C₄ alkyl;
   C₁-C₄ haloalkoxy-C₁-C₄ alkyl;
   C₃-C₈ cycloalkoxy-C₁-C₄ alkyl;
   C₃-C₈ cycloalkyl-C₁-C₄ alkoxy;
   C₁-C₄ alkyl-S—C₁-C₄ alkyl; and
   C₁-C₄ alkyl-SO₂-C₁-C₄ alkyl;
wherein
R^{3a} is selected from H, C₁-C₄ alkyl, and C₁-C₄ alkyl-NR^{3e}R^{3f};
R^{3b} is selected from H and C₁-C₄ alkyl;
R^{3c} is selected from H and C₁-C₄ alkyl;
R^{3d} is selected from C₁-C₄ alkyl; and
R^{3e} and R^{3f} are each independently selected from C₁-C₄ alkyl.

Clause 87. The compound of any one of clauses 1 to 10, 20 to 75, 78, 81, or 86, wherein
R³ is selected from:
   H;
   CN;
   NR^{3a}R^{3b};
   C₁-C₄ alkyl optionally substituted by one or more substituents independently selected from OH and NR^{3c}R^{3d};
   C₃-C₈ cycloalkyl;
   3- to 8-membered heterocycloalkyl having one or more heteroatoms selected from N, O and S;
   phenyl substituted by one or more halogen;
   5- or 6-membered heteroaryl having one or more heteroatoms selected from N, O and S;
   C₁-C₄ alkoxy-C₁-C₄ alkyl;
   C₃-C₈ cycloalkyl-C₁-C₄ alkoxy;
   C₃-C₈ cycloalkoxy-C₁-C₄ alkyl;
   C₁-C₄ haloalkoxy-C₁-C₄ alkyl;
   C₁-C₄ alkyl-S—C₁-C₄ alkyl; and
   C₁-C₄ alkyl-SO₂-C₁-C₄ alkyl;
wherein
R^{3a} is selected from H and C₁-C₄ alkyl;
R^{3b} is selected from C₁-C₄ alkyl;
R^{3c} is selected from H and C₁-C₄ alkyl; and
R^{3d} is selected from C₁-C₄ alkyl.

Clause 88. The compound of any one of clauses 1 to 16, 20 to 76, 78. 79, 81, 82, 84, 86, or 87, wherein
R³ is selected from:
   H;
   CN;
   NR^{3a}R^{3b};
   C₁-C₄ alkyl optionally substituted by OH;
   C₃-C₈ cycloalkyl;
   3- to 8-membered heterocycloalkyl having one or more heteroatoms selected from N, O and S;
   5- or 6-membered heteroaryl having one or more heteroatoms selected from N, O and S;
   C₁-C₄ alkoxy-C₁-C₄ alkyl;
   C₃-C₈ cycloalkyl-C₁-C₄ alkoxy;
   C₃-C₈ cycloalkoxy-C₁-C₄ alkyl;
   C₁-C₄ haloalkoxy-C₁-C₄ alkyl;
   C₁-C₄ alkyl-S—C₁-C₄ alkyl; and
   C₁-C₄ alkyl-SO₂-C₁-C₄ alkyl;
wherein
R^{3a} is H; and
R^{3b} is selected from C₁-C₄ alkyl.

Clause 89. The compound of any preceding clause, wherein
R³ is selected from:
   NR^{3a}R^{3b};
   C₁-C₄ alkyl optionally substituted by OH;
   C₃-C₈ cycloalkyl;
   3- to 8-membered heterocycloalkyl having one or more heteroatoms selected from N, O and S;
   5- or 6-membered heteroaryl having one or more heteroatoms selected from N, O and S;
   C₁-C₄ alkoxy-C₁-C₄ alkyl;
   C₃-C₈ cycloalkyl-C₁-C₄ alkoxy;
   C₃-C₈ cycloalkoxy-C₁-C₄ alkyl;
   C₁-C₄ haloalkoxy-C₁-C₄ alkyl; and
   C₁-C₄ alkyl-S—C₁-C₄ alkyl;
wherein
R^{3a} is H; and
R^{3b} is selected from C₁-C₄ alkyl.

Clause 90. The compound of any preceding clause, wherein R³ is selected from C₁-C₄ alkoxy-C₁-C₄ alkyl.

Clause 91. The compound of any preceding clause, wherein R³ is selected from methoxymethyl.

Clause 92. The compound of any one of clauses 1 to 75, or 78, wherein
R³ is selected from the following groups:

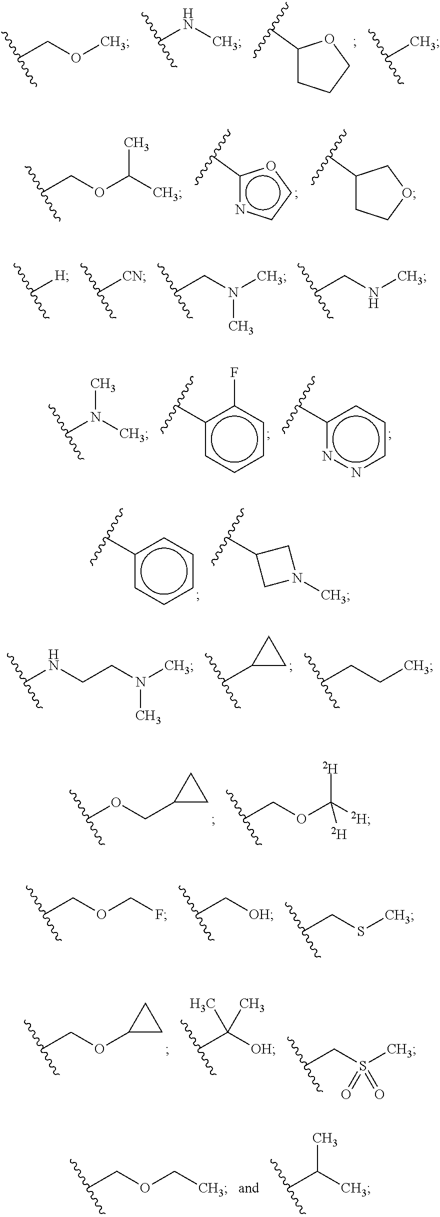

wherein [symbol] indicates the point of attachment to the rest of the compound.

Clause 93. The compound of any one of clauses 1 to 75, 78, 81, or 92, wherein
R³ is selected from the following groups:

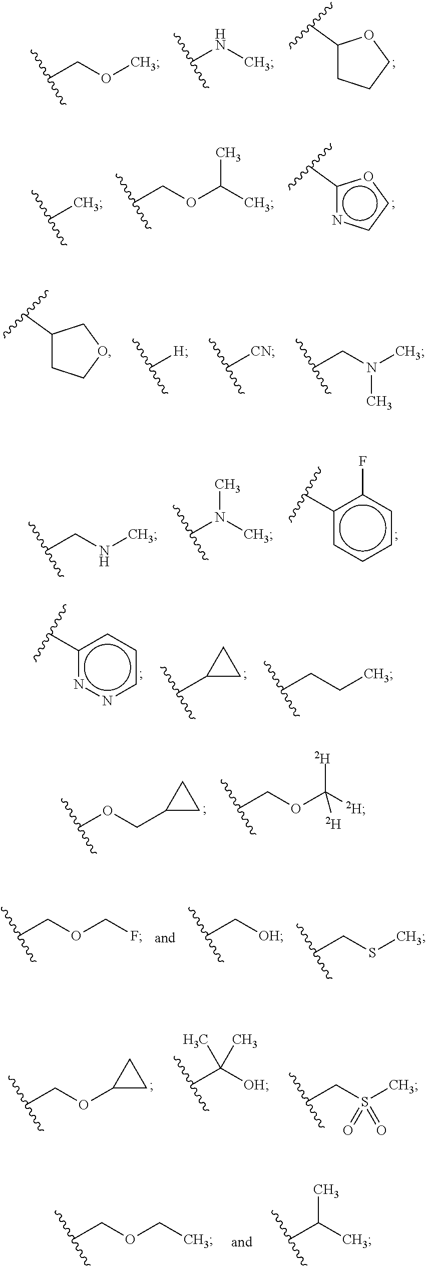

wherein [symbol] indicates the point of attachment to the rest of the compound.

Clause 94. The compound of any one of clauses 1 to 76, 78, 79, 81, 82, 84, 86 to 88, 92, or 93, wherein
R³ is selected from the following groups:

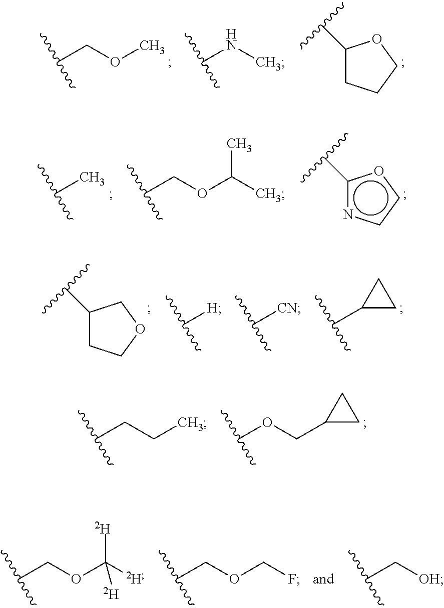

wherein [symbol] indicates the point of attachment to the rest of the compound.

Clause 95. The compound of any one of clauses 1 to 89 or 92 to 94, wherein
R³ is selected from the following groups:

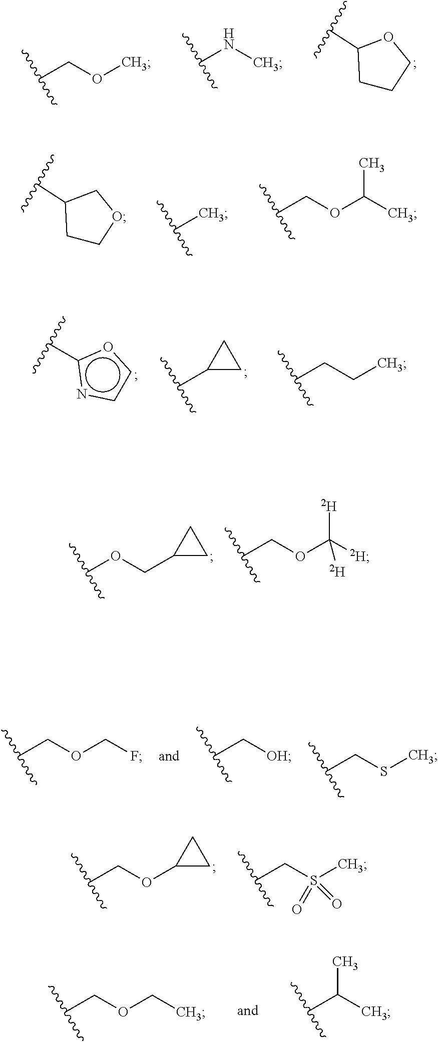

wherein [symbol] indicates the point of attachment to the rest of the compound.

Clause 96. The compound of any one of clauses 1 to 3, or 20 to 95, wherein
R⁴ is selected from C(O)OH, C(O)NR^{4a}R^{4b}, C(S)NR^{4a}R^{4b} and 5- or 6-membered heteroaryl having one or more heteroatoms selected from N, O and S; and
R^{4a} and R^{4b} are each independently selected from H and alkyl.

Clause 97. The compound of any one of clauses 1 to 3 or 20 to 96, wherein
R⁴ is selected from C(O)NR^{4a}R^{4b} and 5- or 6-membered heteroaryl having one or more heteroatoms selected from N, O and S; and
R^{4a} and R^{4b} are each independently selected from H and alkyl.

Clause 98. The compound of any one of clauses 1 to 7 or 20 to 96, wherein
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl; and
R^{4b} is H.

Clause 99. The compound of any one of clauses 1 to 7, 20 to 96 or 98, wherein
R⁴ is selected from C(O)OH, C(O)NR^{4a}R^{4b}, and C(S)NR^{4a}R^{4b};
R^{4a} is selected from H and C₁-C₄ alkyl; and
R^{4b} is H.

Clause 100. The compound of any one of clauses 1 to 10 or 20 to 99, wherein
R⁴ is selected from C(O)OH, C(O)NR^{4a}R^{4b} and C(S)NR^{4a}R^{4b}; and
R^{4a} and R^{4b} are each H.

Clause 101. The compound of any preceding clause, wherein
R⁴ is C(O)NR^{4a}R^{4b}; and
R^{4a} and R^{4b} are each H.

Clause 102. The compound of any one of clauses 1 to 7, or 20 to 96, wherein
R⁴ is selected from the following groups:

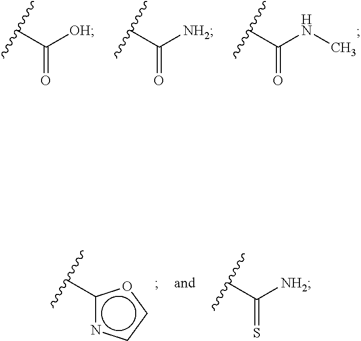

wherein [symbol] indicates the point of attachment to the rest of the compound.

Clause 103. The compound of any one of clauses 1 to 10, 20 to 98, or 102 wherein
R⁴ is selected from the following groups:

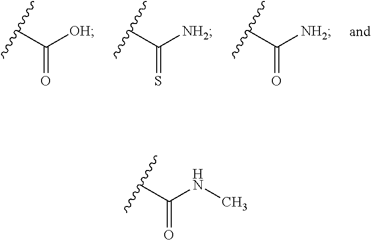

wherein [symbol] indicates the point of attachment to the rest of the compound.

Clause 104. The compound of any one of clauses 1 to 10, 20 to 100, 102, or 103 wherein
R⁴ is selected from the following groups:

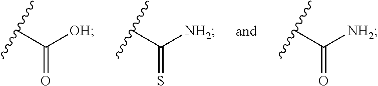

wherein [symbol] indicates the point of attachment to the rest of the compound.

Clause 105. The compound of any preceding clause, wherein
R⁴ is

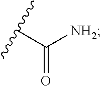 [SMILES: *C(N)=O]

wherein [symbol] indicates the point of attachment to the rest of the compound.

Clause 106. The compound of any one of clauses 1 to 4 or 20 to 105, wherein
R⁵ and R⁶ are each independently selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S; or
R⁵ and R⁶ together with the carbon atoms to which they are attached form a 5- or 6-membered heterocycle having one or more heteroatoms selected from N, O and S.

Clause 107. The compound of any one of clauses 1 to 4 or 20 to 106, wherein
R⁵ and R⁶ are each independently selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S;
or R⁵ and R⁶ together with the carbon atoms to which they are attached form a 5- or 6-membered heterocycle having one or more heteroatoms selected from N and O.

Clause 108. The compound of any one of clauses 1 to 4 or 20 to 107, wherein
R⁵ is selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl, and 5- or 6-membered heteroaryl having one or more heteroatoms selected from N, O and S.

Clause 109. The compound of any one of clauses 1 to 10 or 20 to 108, wherein
R⁵ is selected from H, C₁-C₄ alkyl, and 5- or 6-membered heteroaryl having one or more heteroatoms selected from N, O and S.

Clause 110. The compound of any one of clauses 1 to 13 or 20 to 101, wherein
R⁵ is selected from H and 5- or 6-membered heteroaryl having one or more heteroatoms selected from N, O and S.

Clause 111. The compound of any one of clauses 1 to 10 or 20 to 110, wherein
R⁵ is selected from the following groups:

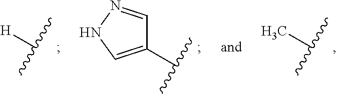

wherein [symbol] indicates the point of attachment to the rest of the compound.

Clause 112. The compound of any one of clauses 1 to 13 or 20 to 111, wherein
R⁵ is selected from the following groups:

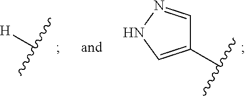

wherein [symbol] indicates the point of attachment to the rest of the compound.

Clause 113. A compound of any preceding clause, wherein
R⁵ is H.

Clause 114. The compound of any one of clauses 1 to 4 or 20 to 113, wherein
R⁶ is selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl, and 5- or 6-membered heteroaryl having one or more heteroatoms selected from N, O and S.

Clause 115. The compound of any one of clauses 1 to 13 or 20 to 114, wherein
R⁶ is selected from H, C₁-C₄ alkyl, C₁-C₄ alkoxy, and C₁-C₄ alkoxy-C₁-C₄ alkyl.

Clause 116. The compound of any one of clauses 1 to 16 or 20 to 115, wherein
R⁶ is selected from H and C₁-C₄ alkyl.

Clause 117. The compound of any one of clauses 1 to 13 or 20 to 115, wherein
R⁶ is selected from the following groups:

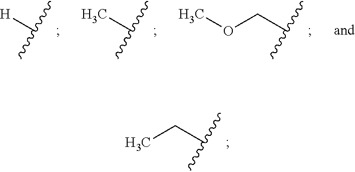

wherein [symbol] indicates the point of attachment to the rest of the compound.

Clause 118. The compound of any one of clauses 1 to 16 or 20 to 117, wherein
R⁶ is H or methyl.

Clause 119. The compound of any preceding clause, wherein
R⁶ is H.

Clause 120. The compound of any one of clauses 1 to 4, or 20 to 106, wherein
R⁵ and R⁶ together with the carbon atoms to which they are attached form a 5- or 6-membered heterocycle having one or more heteroatoms selected from N, O and S.

Clause 121. The compound of any one of clauses 1 to 4, 20 to 107, or 120, wherein
R⁵ and R⁶ together with the carbon atoms to which they are attached form a 5- or 6-membered heterocycle having one or more heteroatoms selected from N and O.

Clause 122. The compound of any one of clauses 1 to 4, 20 to 107, 20, or 121, wherein
R⁵ and R⁶ together with the carbon atoms to which they are attached form a 5-membered heterocycle containing one O atom.

Clause 123. A compound according to any preceding clause, wherein
R⁷, R⁸, R⁹, R¹⁰, R¹¹, and R¹² are each H.

Clause 124. A compound of formula (2):

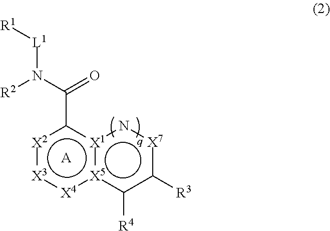

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein:
X¹, X², X³, X⁴, and X⁵ are as defined in clause 1;
X⁷ is selected from CR¹⁰, O, and N; and
q, L¹, R¹, R², R³, R⁴, and R¹⁰ are as defined in any preceding clause;
wherein when q is 0, X⁷ is O or N, and when q is 1, X⁷ is CR¹⁰.

Clause 125. A compound of formula (2a):

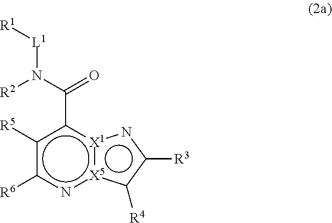

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein X¹, X⁵, L¹, R¹, R², R³, R⁴, R⁵ and R⁶ are as defined in any one of clauses 1 to 123;
wherein one of X¹ and X⁵ is C and the other is N.

Clause 126. A compound of formula (2aa):

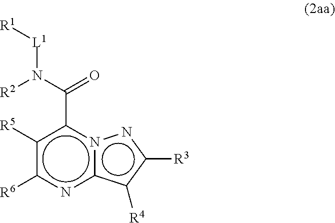

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein L¹, R¹, R², R³, R⁴, R⁵ and R⁶ are as defined in any one of clauses 1 to 123.

Clause 127 A compound of formula (2ab):

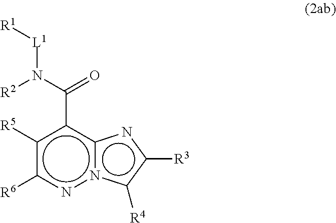

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein L¹, R¹, R², R³, R⁴, R⁵ and R⁶ are as defined in any one of clauses 1 to 123.

Clause 128. A compound of formula (2ac):

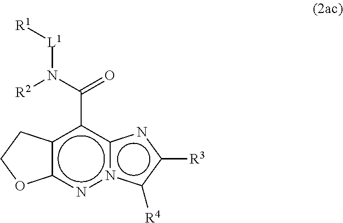

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein L¹, R¹, R², R³ and R⁴ are as defined in any one of clauses 1 to 123.

Clause 129. A compound of formula (2ad):

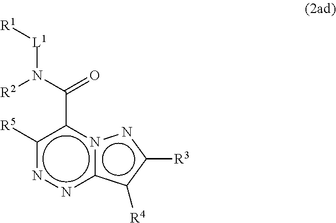

Including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein L¹, R¹, R², R³, R⁴, and R⁵ are as defined in any one of clauses 1 to 123.

Clause 130. A compound of formula (2b):

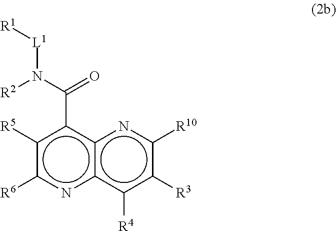

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein q, L¹, R¹, R², R³, R⁴, R⁵, R⁶ and R¹⁰ are as defined in any of clauses 1 to 123.

Clause 131. A compound of formula (3):

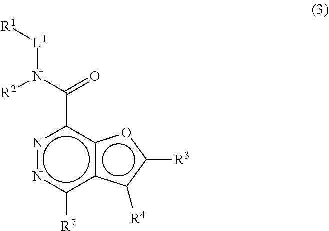

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein L¹, R¹, R², R³, R⁴ and R⁷ are as defined in any one of clauses 1 to 123.

Clause 132. A compound of formula (4):

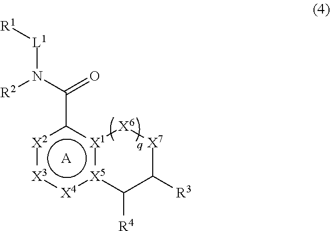

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein:
X¹, X², X³, X⁴, and X⁵ are as defined in clause 1;
X⁶ is selected from CR⁸R⁹ and O;
X⁷ is selected from CR¹¹R¹² and O; and
L¹, q, R¹, R², R³, R⁴, R⁸, R⁹, R¹¹, and R¹² are as defined in any one of clauses 1 to 123;
wherein when q is 0, X⁷ is O; and when X⁷ is O and q is 1, X⁶ is CR⁸R⁹.

Clause 133. A compound of formula (4a):

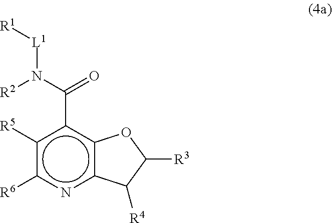

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein:
L¹, q, R¹, R², R³, R⁴, and R⁶ are as defined in any one of clauses 1 to 123.

Clause 134. A compound of formula (4b):

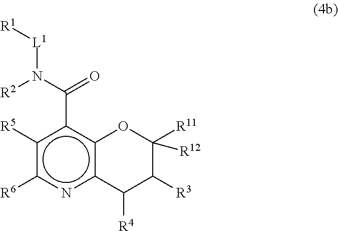

Including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein q, L¹, R¹, R², R³, R⁴, R⁵, R⁶, R¹¹ and R¹² are as defined in any one of clauses 1 to 123.

Clause 135. A compound of formula (4c):

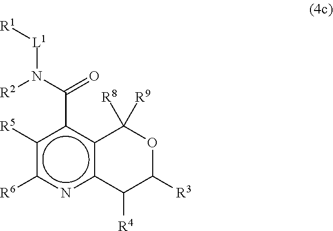

including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein q, L¹, R¹, R², R³, R⁴, R⁵, R⁶, R⁸ and R⁹ are as defined in any one of clauses 1 to 123.

Clause 136. The compound of clause 1, wherein the compound is selected from compounds 1 to 242 as shown in Table 1.

Clause 137. The compound of any of clauses 1 to 136 for use in medicine.

Clause 138. The compound for use of clause 137, wherein the use is in the treatment or prophylaxis of a cancer or proliferative disease or disorder.

Clause 139. The compound for use of clause 137, wherein the use is in the treatment or prophylaxis of fibrotic disease or inflammatory disease.

Clause 140. A pharmaceutical composition comprising a compound of any of clauses 1 to 136, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier.

Clause 141. The pharmaceutical composition of clause 140 for use according to any of clauses 137 to 140.

Clause 142. A method of treatment comprising administration of a therapeutically effective amount of a compound of any of clauses 1 to 136, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, to a patient in need thereof.

Clause 143. The method of clause 142, wherein the patient has a cancer or proliferative disease or disorder.

Clause 144. The method of clause 143, wherein the patient has fibrotic disease or inflammatory disease.

Clause 145. A method of treating a cancer or proliferative disease or disorder, comprising administering one or more compound(s) according to any of clauses 1 to 136, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, to a subject in need thereof.

Clause 146. A method of treating fibrotic disease or inflammatory disease, comprising administering one or more compound(s) according to any of clauses 1 to 136, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, to a subject in need thereof.

Clause 147. The use of a compound according to any of clauses 1 to 136, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, for the manufacture of a medicament.

Clause 148. The use of a compound according to any of clauses 1 to 136, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, for the manufacture of a medicament for the treatment or prophylaxis of a cancer or proliferative disease or disorder.

Clause 149. The use of a compound according to any of clauses 1 to 136, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, for the manufacture of a medicament for the treatment or prophylaxis of fibrotic disease or inflammatory disease.

As used herein, it should be appreciated that the expression “selected from A, B or C” (or analogous) is intended to mean that a selection from the group of A, B and C is made; and that the group may comprise or consist of the specified elements A, B and C. Similarly, the phrase “selected from A, B and C”” (or analogous) is intended to mean that a selection from the group of A, B and C is made. Thus, no difference is intended by the inclusion of ‘or’ or ‘and’ in such phrases, unless otherwise stated or clear from the context of the disclosure. As will be appreciated, the phrase “selected from at least one of A, B or C” and the phrase “selected from at least one of A, B and C” are intended to include all combinations of one or more of the specified elements and no difference is intended by the inclusion of ‘or’ or ‘and’, unless otherwise stated or clear from the context of the disclosure. Generally, it is intended that a recited list of alternatives, such as defined by e.g. the group of “A, B and C”, means that the group is exclusive of additional alternatives, and so in preferred embodiments can be read to mean “selected from the group consisting of A, B and C” (or analogously), unless otherwise stated.

Many modifications may be made to the above examples without departing from the scope of the present invention as defined in the accompanying claims.

1. A compound of formula (1): 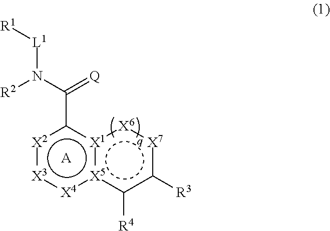
including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein:
Q is O or S;
X¹ is selected from C and N;
X² is selected from CR⁵ and N;
X³ is selected from CR⁶ and N;
X⁴ is selected from CR⁷ and N;
X⁵ is selected from C and N;
wherein X¹, X², X³, X⁴ and X⁵ are selected so that the ring labelled A is aromatic;
X⁶ is selected from CR⁸R⁹, O and N;
X⁷ is selected from CR¹⁰, CR¹¹R¹², O and N;
q is 0 or 1;
wherein when q is 0, X⁷ is O or N; and when X⁷ is O or N and q is 1, X⁶ is CR⁸R⁹;
L¹ is a bond, C₁-C₆ alkylene, C₁-C₆ haloalkylene, C₃-C₆ cycloalkylene, or a 5- or 6-membered heterocycloalkyl having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, C₉-C₁₀ bicyclic aryl, 5- or 6-membered heteroaryl having one or more ring members selected from N, O, S and SO₂, 8- to 10-membered bicyclic heteroaryl having one or more ring members selected from N, O, S and SO₂, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is selected from H, C₁-C₄ alkyl and C₃-C₈ cycloakyl;
or R¹, R² and L¹ and the nitrogen atom to which R² and L¹ are attached form a 3- to 8-membered heterocycloalkyl having one or more heteroatoms selected from N, O and S, 8- to 11-membered bicyclic heterocycloalkyl having one or more heteroatoms selected from N, O and S, 5- or 6-membered heteroaryl having one or more heteroatoms selected from N, O and S, or 8- to 10-membered bicyclic heteroaryl having one or more heteroatoms selected from N, O and S, wherein said heterocycloalkyl, bicyclic heterocycloalkyl, heteroaryl and bicyclic heteroaryl are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy and C₁-C₄ alkyl-NR^{1a}R^{1b};
R³ is selected from H, OH, CN, NR^{3a}R^{3b}, C₁-C₄ alkyl, C₃-C₈ cycloalkyl, 3- to 8-membered heterocycloalkyl having one or more heteroatoms selected from N, O and S, phenyl, and 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, CN, C₁-C₄ alkyl, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} and R^{4b} are each independently selected from H and C₁-C₄ alkyl;
R⁵ and R⁶ are each independently selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S; or
R⁵ and R⁶ together with the carbon atoms to which they are attached form a 5- or 6-membered heterocycloalkyl having one or more heteroatoms selected from N, O and S; and
R⁷, R⁸, R⁹, R¹⁰, R¹¹, and R¹² are each independently selected from H, halogen, OH, CN, C₁-C₄ alkyl, C₁-C₄ haloalkyl, C₁-C₄ alkoxy, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S.

2. The compound of claim 1, wherein
L¹ is a bond, C₁-C₆ alkylene, C₁-C₆ haloalkylene, C₃-C₆ cycloalkylene, or a 5- or 6-membered heterocycloalkyl having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, C₉-C₁₀ bicyclic aryl, 5- or 6-membered heteroaryl having one or more ring members selected from N, O, S and SO₂, 8- to 10-membered bicyclic heteroaryl having one or more ring members selected from N, O, S and SO₂, phenoxy, and 5- or 6-membered heteroaryloxy having one or more heteroatoms selected from N, O and S, wherein said alkyl, cycloalkyl, bicyclic cycloalkyl, heterocycloalkyl, bicyclic heterocycloalkyl, phenyl, bicyclic aryl, heteroaryl, bicyclic heteroaryl, phenoxy and heteroaryloxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, benzyl, phenoxy, halophenoxy, 5- or 6-membered heteroaryloxy having one or more heteroatoms selected from N, O and S, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is selected from H, C₁-C₄ alkyl and C₃-C₈ cycloakyl;
R³ is selected from H, OH, CN, NR^{3a}R^{3b}, C₁-C₄ alkyl, C₃-C₈ cycloalkyl, 3- to 8-membered heterocycloalkyl having one or more heteroatoms selected from N, O and S, phenyl, and 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, CN, C₁-C₄ alkyl, and NR^{3c}R^{3d};
R^{3a} is selected from H, C₁-C₄ alkyl, C₁-C₄ alkyl-OH, and C₁-C₄ alkyl-NR^{3e}R^{3f};
R^{3b}, R^{3c}, R^{3d}, R^{3e} and R^{3f} are each independently selected from H and C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} and R^{4b} are each independently selected from H and C₁-C₄ alkyl;
R⁵ and R⁶ are each independently selected from H, halogen, OH, CN, C₁-C₄ alkyl, C₁-C₄ haloalkyl, C₁-C₄ alkoxy, C₁-C₄ alkoxy-C₁-C₄ alkyl, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S; or
R⁵ and R⁶ together with the carbon atoms to which they are attached form a 5- or 6-membered heterocycloalkyl having one or more heteroatoms selected from N, O and S; and
R⁷, R⁸, R⁹, R¹⁰, R¹¹, and R¹² are each independently selected from H, halogen, OH, CN, C₁-C₄ alkyl, C₁-C₄ haloalkyl, C₁-C₄ alkoxy, C₃-C₈ cycloalkyl, 3- to 8-membered heterocycloalkyl having one or more heteroatoms selected from N, O and S, phenyl and 5- or 6-membered heteroaryl having one or more heteroatoms selected from N, O and S.

3. The compound of claim 1 or claim 2, wherein
L¹ is a bond, C₁-C₆ alkylene, C₁-C₆ haloalkylene, C₃-C₆ cycloalkylene, or a 5- or 6-membered heterocycloalkyl having one or more heteroatoms selected from N, O and S;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, 8- to 10-membered bicyclic heteroaryl having one or more ring members selected from N, O, S and SO₂, and phenoxy, wherein said alkyl, cycloalkyl, heterocycloalkyl, bicyclic cycloalkyl, bicyclic heterocycloalkyl, phenyl, heteroaryl, bicyclic heteroaryl, and phenoxy are each optionally substituted by one or more substituents independently selected from halogen, OH, CN, NR^{1a}R^{1b}, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 3- to 8-membered heterocycloalkyl having one or more heteroatoms selected from N, O and S, 5- or 6-membered heteroaryl having one or more heteroatoms selected from N, O and S, benzyl, halophenoxy, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH, C₁-C₄ alkyl, and NR^{3c}R^{3d};
R^{3a} is selected from H, C₁-C₄ alkyl, and C₁-C₄ alkyl-NR^{3e}R^{3f},
R^{3b} is selected from H and C₁-C₄ alkyl;
R^{3c} is selected from H and C₁-C₄ alkyl;
R^{3d} is selected from C₁-C₄ alkyl;
R^{3e} and R^{3f} are each independently selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

4. The compound of any preceding claim, wherein
L¹ is a bond, C₁-C₆ alkylene, C₁-C₆ haloalkylene, or C₃-C₆ cycloalkylene;
R¹ is selected from C₁-C₄ alkyl, C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, 8- to 10-membered bicyclic heteroaryl having one or more ring members selected from N, O, S and SO₂, and phenoxy, wherein said alkyl, cycloalkyl, heterocycloalkyl, bicyclic cycloalkyl, bicyclic heterocycloalkyl, phenyl, heteroaryl, bicyclic heteroaryl, and phenoxy are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, C₃-C₈ cycloalkyl, 5- or 6-membered heteroaryl having one or more heteroatoms selected from N, O and S, benzyl, and C₁-C₄ alkyl substituted by a 5- or 6-membered heterocycloalkyl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH and NR^{3c}R^{3d};
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from C(O)OH, C(O)NR^{4a}R^{4b}, C(S)NR^{4a}R^{4b} and 5- or 6-membered heteroaryl having one or more heteroatoms selected from N, O and S;
R^{4a} is selected from H and C₁-C₄ alkyl;
R^{4b} is H;
R⁵ is selected from H, C₁-C₄ alkyl, and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

5. The compound of any preceding claim, wherein
L¹ is a bond, C₁-C₆ alkylene, C₁-C₆ haloalkylene, or C₃-C₆ cycloalkylene;
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, CN, OH, C₁-C₄ alkyl, C₁-C₄ haloalkyl, C₁-C₄ alkoxy, C₁-C₄ haloalkoxy, C₁-C₄ alkyl-NR^{1a}R^{1b}, and 5- or 6-membered heteroaryl having one or more heteroatoms selected from N, O and S;
R^{1a} and R^{1b} are each independently selected from H and C₁-C₄ alkyl;
R² is H;
R³ is selected from H, CN, NR^{3a}R^{3b}, C₁-C₄ alkyl, C₃-C₈ cycloalkyl, 3- to 8-membered heterocycloalkyl having one or more heteroatoms selected from N, O and S, phenyl, 5- or 6-membered heteroaryl having one or more heteroatoms selected from N, O and S, C₁-C₄ alkoxy-C₁-C₄ alkyl, C₁-C₄ haloalkoxy-C₁-C₄ alkyl, C₃-C₈ cycloalkoxy-C₁-C₄ alkyl, C₃-C₈ cycloalkyl-C₁-C₄ alkoxy, C₁-C₄ alkyl-S—C₁-C₄ alkyl, and C₁-C₄ alkyl-SO₂-C₁-C₄ alkyl, wherein said alkyl, cycloalkyl, heterocycloalkyl, phenyl and heteroaryl are each optionally substituted by one or more substituents independently selected from halogen, OH and NR^{3c}R^{3d};
R^{3a} is selected from H and C₁-C₄ alkyl;
R^{3b} is selected from C₁-C₄ alkyl;
R^{3c} is selected from H and C₁-C₄ alkyl;
R^{3d} is selected from C₁-C₄ alkyl;
R⁴ is selected from the following groups: 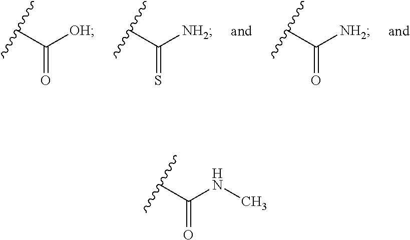
wherein [symbol] indicates the point of attachment to the rest of the compound;
R⁵ is selected from H and 5- or 6-membered heteroaryl having one or more heteroatoms selected from N, O and S;
R⁶ is selected from H, C₁-C₄ alkyl, C₁-C₄ alkoxy, and C₁-C₄ alkoxy-C₁-C₄ alkyl; and
R⁷, R⁸, R⁹, R¹⁰, R¹¹, and R¹² are each H.

6. The compound of any preceding claim, wherein
L¹ is a bond, C₁-C₆ alkylene, or C₃-C₆ cycloalkylene;
R¹ is selected from C₃-C₈ cycloalkyl, C₇-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, heterocycloalkyl, bicyclic cycloalkyl, bicyclic heterocycloalkyl, phenyl, heteroaryl, and bicyclic heteroaryl are each optionally substituted by one or more substituents independently selected from halogen, OH, C₁-C₄ alkyl, C₁-C₄ haloalkyl, C₁-C₄ alkoxy, and C₁-C₄ haloalkoxy;
R² is H;
R³ is selected from H, CN, NR^{3a}R^{3b}, C₁-C₄ alkyl optionally substituted by OH, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₁-C₄ alkoxy-C₁-C₄ alkyl, C₃-C₈ cycloalkyl-C₁-C₄ alkoxy, C₁-C₄ haloalkoxy-C₁-C₄ alkyl, C₁-C₄ alkyl-S—C₁-C₄ alkyl, and C₁-C₄ alkyl-SO₂-C₁-C₄ alkyl;
R^{3a} is H;
R^{3b} is selected from C₁-C₄ alkyl;
R⁴ is selected from the following groups: 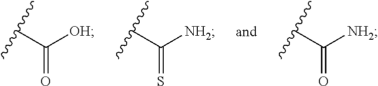
wherein [symbol] indicates the point of attachment to the rest of the compound;
R⁵ is H;
R⁶ is selected from H and C₁-C₄ alkyl; and
R⁷, R⁸, R⁹, R¹⁰, R¹¹, and R¹² are each H.

7. The compound of any preceding claim, wherein
L¹ is either a bond or C₁-C₆ alkylene;
R¹ is selected from C₃-C₈ cycloalkyl, C₈-C₁₁ bicyclic cycloalkyl, 3- to 8-membered heterocycloalkyl having one or more ring members selected from N, O, S and SO₂, 8- to 11-membered bicyclic heterocycloalkyl having one or more ring members selected from N, O, S and SO₂, phenyl, 5- or 6-membered heteroaryl having one or more ring members selected from N, O, S and SO₂, and 8- to 10-membered bicyclic heteroaryl having one or more ring members selected from N, O, S and SO₂, wherein said cycloalkyl, bicyclic cycloalkyl, heterocycloalkyl, bicyclic heterocycloalkyl, phenyl, heteroaryl, and bicyclic heteroaryl are each optionally substituted by one or more substituents independently selected from halogen, OH, C₁-C₄ alkyl, C₁-C₄ haloalkyl, C₁-C₄ alkoxy, and C₁-C₄ haloalkoxy;
R² is H;
R³ is selected from NR^{3a}R^{3b}, C₁-C₄ alkyl optionally substituted by one or more substituents independently selected from OH and NR^{3c}R^{3d}, C₃-C₈ cycloalkyl, 3- to 8-membered heterocycloalkyl having one or more heteroatoms selected from N, O and S, 5- or 6-membered heteroaryl having one or more heteroatoms selected from N, O and S, C₃-C₈ cycloalkoxy-C₁-C₄ alkyl, C₁-C₄ alkoxy-C₁-C₄ alkyl, C₁-C₄ haloalkoxy-C₁-C₄ alkyl, C₃-C₈ cycloalkyl-C₁-C₄ alkoxy, and alkyl-S—C₁-C₄ alkyl;
R^{3a} is H;
R^{3b} is selected from C₁-C₄ alkyl;
R⁴ is selected from the following groups: 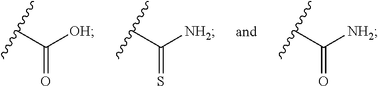
wherein [symbol] indicates the point of attachment to the rest of the compound R⁵, R⁷, R⁸, R⁹, R¹⁰, R¹¹, and R¹² are each H; and
R⁶ is selected from H and C₁-C₄ alkyl.

8. The compound of any preceding claim, wherein Q is O.

9. The compound of any preceding claim, wherein L¹ is either a bond or methylene.

10. The compound of any preceding claim, wherein
R¹ is selected from:
C₃-C₈ cycloalkyl optionally substituted by one or more substituents independently selected from halogen and C₁-C₄ haloalkoxy;
C₈-C₁₁ bicyclic cycloalkyl optionally substituted by one or more substituents independently selected from halogen, OH, C₁-C₄ alkyl and C₁-C₄ alkoxy;
3- to 8-membered heterocycloalkyl having one or more ring members selected from N, O, S and SO₂;
8- to 11-membered bicyclic heterocycloalkyl having one or more ring members selected from N, O, S and SO₂, optionally substituted by one or more substituents independently selected from halogen;
phenyl optionally substituted by one or more substituents independently selected from halogen and C₁-C₄ alkyl;
5- or 6-membered heteroaryl having one or more ring members selected from N, O, S and SO₂, optionally substituted by one or more substituents independently selected from C₁-C₄ alkyl and C₁-C₄ haloalkyl; and
8- to 10-membered bicyclic heteroaryl having one or more ring members selected from N, O, S and SO₂, optionally substituted substituted by one or more substituents independently selected from halogen and C₁-C₄ alkyl.

11. The compound of any preceding claim, wherein
R² is H.

12. The compound of any preceding claim, wherein
R³ is selected from:
NR^{3a}R^{3b};
C₁-C₄ alkyl optionally substituted by OH;
C₃-C₈ cycloalkyl;
3- to 8-membered heterocycloalkyl having one or more heteroatoms selected from N, O and S;
5- or 6-membered heteroaryl having one or more heteroatoms selected from N, O and S;
C₁-C₄ alkoxy-C₁-C₄ alkyl;
C₃-C₈ cycloalkyl-C₁-C₄ alkoxy;
C₃-C₈ cycloalkoxy-C₁-C₄ alkyl;
C₁-C₄ haloalkoxy-C₁-C₄ alkyl; and
C₁-C₄ alkyl-S—C₁-C₄ alkyl;
wherein
R^{3a} is H; and
R^{3b} is selected from C₁-C₄ alkyl.

13. The compound of any preceding claim, wherein R³ is selected from C₁-C₄ alkoxy-C₁-C₄ alkyl.

14. The compound of any one of claims 1 to 3 or 8 to 13, wherein
R⁴ is selected from the following groups: 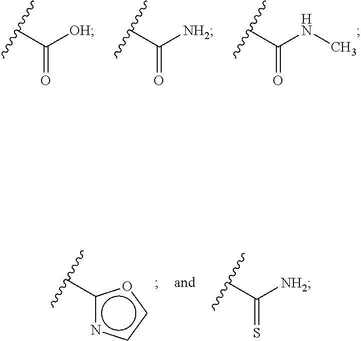
wherein [symbol] indicates the point of attachment to the rest of the compound.

15. A compound of any preceding claim, wherein
R⁵ is H.

16. The compound of any preceding claim, wherein
R⁶ is H or methyl.

17. A compound according to any preceding claim, wherein
R⁷, R⁸, R⁹, R¹⁰, R¹¹, and R¹² are each H.

18. A compound of formula (2a): 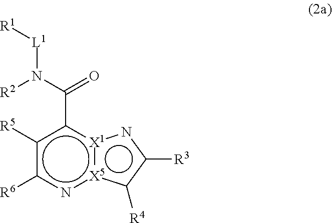
including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein X¹, X⁵, L¹, R¹, R², R³, R⁴, R⁵ and R⁶ are as defined in any one of claims 1 to 16;
wherein one of X¹ and X⁵ is C and the other is N.

19. A compound of formula (2aa): 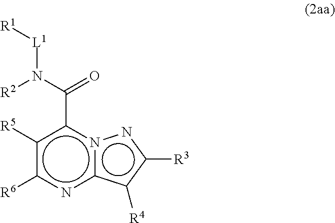
including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein L¹, R¹, R², R³, R⁴, R⁵ and R⁶ are as defined in any one of claims 1 to 16.

20. A compound of formula (2ab): 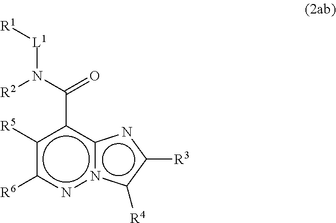
including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein L¹, R¹, R², R³, R⁴, R⁵ and R⁶ are as defined in any one of claims 1 to 16.

21. A compound of formula (2ad): 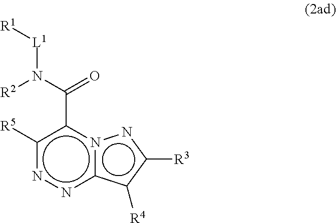
Including any stereoisomer or mixture of stereoisomers including diastereomers and enantiomers, tautomer, isotopic form, pharmaceutically acceptable salt, solvate, prodrug, or pharmaceutically active metabolite thereof, or combinations thereof, wherein L¹, R¹, R², R³, R⁴, and R⁵ are as defined in any one of claims 1 to 16.

22. The compound of claim 1, wherein the compound is selected from compounds 1 to 242.

23. The compound of any of claims 1 to 22 for use in medicine.

24. The compound for use of claim 23, wherein the use is in the treatment or prophylaxis of a cancer or proliferative disease or disorder.

25. The compound for use of claim 23, wherein the use is in the treatment or prophylaxis of fibrotic disease or inflammatory disease.

26. A pharmaceutical composition comprising a compound of any of claims 1 to 22, or a stereoisomer or mixture of stereoisomers including diastereomers and enantiomers, tautomer, isotopic form, pharmaceutically acceptable salt, solvate, prodrug, or pharmaceutically active metabolite thereof, or combinations thereof, and at least one pharmaceutically acceptable diluent, excipient or carrier.

27. The pharmaceutical composition of claim 26 for use according to any of claims 23 to 25.
